(19) United States
(12) Patent Application Publication
(10) Pub. No.: US 2025/0046465 A1
(43) Pub. Date: Feb. 6, 2025

(54) BREAST CANCER RISK ASSESSMENT

(71) Applicant: Genetic Technologies Limited, Victoria (AU)

(72) Inventors: Richard Allman, Victoria (AU); Gillian Sue DITE, Victoria (AU)

(73) Assignee: Genetic Technologies Limited, Victoria (AU)

(21) Appl. No.: 18/717,288

(22) PCT Filed: Dec. 6, 2022
(86) PCT No.: PCT/AU2022/051459

§ 371 Date: Jun. 6, 2024

(30) Foreign Application Priority Data
Dec. 7, 2021 (AU) 2021903955

(51) Int. Cl. G16H 50/30; G16H 10/40; G16H 10/60

(52) U.S. Cl. CPC G16H 50/30; G16H 10/40; G16H 10/60

(57) ABSTRACT

The present disclosure relates to methods and systems for assessing the risk of a human female subject for developing breast cancer.

### FIELD OF THE INVENTION

The present disclosure relates to methods and systems for assessing the risk of a human female subject for developing breast cancer.

### BACKGROUND OF THE INVENTION

For decades, women have been told that they have an increased risk of breast cancer if they have a family history of the disease (Collaborative Group on Hormonal Factors in Breast Cancer, 2001), and more recently, women have been told that they have increased risk of breast cancer if they have mammographically dense breasts (Cappello et al., 2019) among other factors (Hopper et al., 2018). Genetic risk derived from common changes throughout the genome can also be used to predict risk of breast cancer (Mavaddat et al., 2015), but this information is not readily available to women.

This fragmented approach to risk prediction can result in women being given inaccurate risk information and can mean that women get conflicting and confusing advice.

About 22% of women report having at least one first-degree relative with breast cancer (Nickson et al., 2018; Ziogas et al., 2000) and 20% report having a family history in at least one second-degree relative (Ziogas et al., 2000). The magnitude of familial risk depends on the closeness of the genetic relationship, the age at diagnosis of the affected relative and the age of the at-risk woman. Familial risk is best captured by algorithms that model major genes and underlying genetic risk using multi-generational pedigree data (Antoniou et al., 2008; Tyrer et al., 2004; Berry et al., 1997). Cancer family clinics use these tools when managing the small proportion of women who have several affected relatives and where there is the time and commitment to collect and validate extensive family cancer data. However, this approach is not feasible on a population basis using current methods.

Mammographic density is the bright regions on a mammogram and can be measured on a continuous scale using a computer-assisted method (e.g. Cumulus) (Byng et al., 1998) or assigned to categories by radiologists' visual inspection (e.g. BI-RADS) (Sak et al., 2015). Age- and body mass index-adjusted percent mammographic density is strongly associated with risk of breast cancer (Boyd et al., 2002). The issue of mammographically dense breasts has become important, especially in the United States, where mammographically dense breasts are defined as BI-RADS categories C or D (Sickles and Bassett, 2013), which are the equivalent of having a percent mammographic density greater than 50%. Over 40% of women in the United States have dense breasts, more so at young ages.

Mammography clinics in most states of the United States are now required by law to notify women if they have mammographically dense breasts. Women receiving a dense breast notification are encouraged to discuss this and other breast cancer risk factors with their healthcare provider to decide if additional screening options are necessary. Unfortunately, most women are unaware of the other risk factors and most healthcare professionals are reluctant to pursue more aggressive screening modalities based on mammographic density alone.

The first major susceptibility genes for breast cancer (BRCA1 and BRCA2) were discovered over 20 years ago (Wendt and Margolin, 2019), and since then TP53, STK11, CD1 and PTEN have been identified. The approximately 1 in 600 women with mutations in BRCA1 or BRCA2 have, on average, a lifetime breast cancer risk of >50% (Kuchenbaecker et al., 2017), much greater than the population average risk of about 10%. The rarity of these mutations means they have limited impact on population health.

A single-nucleotide polymorphism (SNP) is a nucleotide substitution that has an allele frequency of >1% in the population. Large collaborative studies have identified SNPs that are independently associated with breast cancer risk. These associations are small, with the odds ratio per risk allele typically being 1.1 or less (Michailidou et al., 2017), and empirically appear to multiply. These susceptibility SNPs can be used to create a polygenic risk score (PRS) that can predict risk of disease.

Despite available tools, there is a need for improved comprehensive breast cancer risk assessment methods.

### SUMMARY OF THE INVENTION

The present inventors have developed a model to accurately predict breast cancer risk for unaffected women. This approach is of practical importance for both clinical and population health. Women at very high risk of breast cancer can be identified and given more frequent or alternative modes of screening, and women at very low risk of breast cancer can be identified as candidates for less frequent screening, with a concomitant financial benefit to population-based screening programs.

In a first aspect, the present invention provides a computer-implemented method for assessing the risk of a human female subject developing breast cancer, the method comprising:
determining a genetic risk score based on genetic data derived from a biological sample taken from the female subject;
determining a mammographic density risk score based on data derived from a clinical assessment of the mammographic density of the female subject;
determining a first-degree family history risk score and a second-degree family history risk score based on family history data of the female subject;
determining a body mass index risk score based on the body mass index and menopause status of the subject;
calculating a risk modifier score, the risk modifier score being a function of the polygenetic risk score, mammographic density risk score, first-degree family history risk score, second-degree family history risk score and body mass index risk score;
calculating at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculating the female subject's absolute risk of breast cancer in the next N years, and/or in the subjects full lifetime, using the cumulative risk scores.

In an embodiment, the genetic risk score is determined by ln(PRS)×β_{PRS}, where the PRS is the polygenic risk score that represents the female subject's genetic predisposition to breast cancer, and β_{PRS} is the natural logarithm of the relative risk associated with a one-unit increase in ln(PRS).

In an embodiment, the genetic risk score is determined using at least 50, at least 100, at least 150, at least 200, at least 250, at least 300, at least 313, at least 350, at least 400, at least 450, at least 500, at least 550, at least 600, at least 650, at least 700, at least 750, at least 800, at least 825, at least 850, at least 3,820 or all of the polymorphism provided in one or more of Tables 8 to 18, or a polymorphism in linkage disequilibrium with one or more thereof.

In an embodiment, the genetic risk score is determined using the 313 polymorphism of Table 17.

In an embodiment, the genetic risk score is determined using the 3,820 polymorphisms of Table 18.

In an embodiment, the first-degree family history risk score (fh_risk_1) is based on the subjects age and the number and youngest age affected of the subjects first-degree relatives with invasive breast cancer., such as determined using Table 1.

In an embodiment, the first-degree family history risk score is ln(fh_risk_1).

In an embodiment, the second-degree family history risk score (fh_risk_2) is based on the subjects age and the subject's number of second-degree relatives with invasive breast cancer, such as determined using Table 2.

In an embodiment, the second-degree family history risk score is ln(fh_risk_2). In an embodiment, the body mass index risk score is determined using intercept_{PRE}+((ln(bmi/5)−ln(mean_{BMI}/5))×β_{PRE}) if pre-menopausal, or intercept_{POST}+((ln(bmi/5)−ln(mean_{BMI}/5))×β_{POST}) if post-menopausal, where mean_{BMI} is the mean body mass index in the population in which the test is used, intercept_{PRE} is the equation intercept for pre-menopausal women and intercept_{POST} is the equation intercept for post-menopausal women.

In an embodiment, the mammographic density risk score is determined using (sqrt(percent_density)−sqrt(mean_{DENS}))×β_{DENS}, where mean_{DENS} is the mean percent density in the population in which the test is used, and β_{DENS} is the natural logarithm of the relative risk associated with a one-unit increase in the square root of percent density.

In an embodiment, the risk modifier score (risk_mod1) is determined using:

$$lnrisk 1 = ln ( PRS ) \times {β}_{PRS} ( sqrt ( percent\_density ) - sqrt ( {mean}_{DENS} ) ) \times {β}_{DENS} + ln ( fh\_risk \_ 1 ) + ln ( fh\_risk \_ 2 ) + {intercept}_{PRE} + ( ( ln ( bmi / 5 ) - ln ( {mean}_{BMIW} / 5 ) ) \times {β}_{PRE} ) if pre - menopausal + {intercept}_{POST} + ( ( ln ( bmi / 5 ) - ln ( {mean}_{BMI} / 5 ) ) \times {β}_{POST} ) if post - menopausal$$

then:

risk_mod1=e^{lnrisk1}.

In an embodiment, the at least two cumulative risk scores are selected from:

$$cumul1\_b = 1 - {e}^{- risk \_ mod 1 \times incid \_ b} \\ cumul1\_b \_N = 1 - {e}^{- risk\_mod 1 \times incid \_ b \_ N} \\ cumul1\_full \_life = 1 - {e}^{- risk \_ mod 1 \times incid \_ full \_ life}$$

where:
N is the number of years of risk to calculate
incid_b=is the ethnicity-specific cumulative breast cancer incidence up to current age,
incid_bN5=is the ethnicity-specific cumulative breast cancer incidence up to current age plus 5N years, and
incid_full_life is the ethnicity-specific cumulative breast cancer incidence up to 90 years.

In an embodiment, breast cancer risk score is determined using either of:

$$brisk 1 \_N = \frac{( cumul 1 \_b \_N - cumul 1 \_b )}{( 1 - cumul 1 \_b )} \\ \begin{matrix}or \\ brisk 1 \_rem \_life = \frac{( cumul 1 \_full \_life - cumul 1 \_b )}{( 1 - cumul 1 \_b )}\end{matrix}$$

In an embodiment, the mammographic density risk score is a BI-RADS category.

In an embodiment, the risk modifier score (risk_mod2) is determined using:

$$Inrisk 1 = ln ( PRS ) \times {β}_{PRS} + X if BI ‐ RADS category is A or B + Y if BI ‐ RADS category is C or D + ln ( {fh\_risk}_{-} 1 ) + ln ( {fh\_risk}_{-} 2 ) + {intercept}_{PRE} + ( ( ln ( bmi / 5 ) - ln ( {mean}_{BMI} / 5 ) ) \times {β}_{PRE} ) if pre ‐ menopausal + {intercept}_{POST} + ( ( ln ( bmi / 5 ) - ln ( {mean}_{BMI} / 5 ) ) \times {β}_{POST} ) if post ‐ menopausal$$

wherein, X is between −0.05 and −0.25, or between −0.1 and −0.2 or is −0.15, and Y is between 0.14 and 0.34, or between 0.19 and 0.29, or is 0.24,
   then:

risk_mod2=e^{lnrisk2}.

In an embodiment, the at least two cumulative risk scores are selected from:

$$cumul 2 \_b = 1 - {e}^{- risk\_mod 2 \times incid \_ b} \\ cumul 2 \_ b \_ N = 1 - {e}^{- risk \_ mod 2 \times incid \_ b \_ N} \\ cumul 2 \_full \_life = 1 - {e}^{- risk \_ mod 2 \times incid \_ full \_ life}$$

where:
N is the number of years of risk to calculate,
incid_b=is the ethnicity-specific cumulative breast cancer incidence up to current age,
incid_b_N5=is the ethnicity-specific cumulative breast cancer incidence up to current age plus 5N years, and
incid_full_life is the ethnicity-specific cumulative breast cancer incidence up to 90 years (i.e. full-lifetime risk).

In an embodiment, the breast cancer risk score is determined using either of:

$$brisk 2 \_N = \frac{( cumul 2 \_b \_N - cumul 2 \_b )}{( 1 - cumul 2 \_b )} \\ \begin{matrix}or \\ brisk 2 \_rem \_life = \frac{( cumul 2 \_full \_life - cumul 2 \_b )}{( 1 - cumul 2 \_b )} .\end{matrix}$$

In an embodiment, the method further comprises comparing the score to a pre-determined threshold.

In an embodiment, the method further comprises outputting the breast cancer risk score.

In second aspect, the present invention provides a computer-implemented method for assessing the risk of a human female subject developing breast cancer, the method comprising:
determining a mammographic density risk score based on data derived from a clinical assessment of the mammographic density of the female subject;
determining a first-degree family history risk score and a second-degree family history risk score based on family history data of the female subject;
determining a body mass index risk score based on the body mass index and menopause status of the subject;
calculating a risk modifier score, the risk modifier score being a function of the mammographic density risk score, first-degree family history risk score, second-degree family history risk score and body mass index risk score;
calculating at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculating the female subject's absolute risk of breast cancer in the next N years, and/or in the subjects full lifetime, using the cumulative risk scores.

The embodiments above are also relevant to this method apart from the determining a genetic risk score. Thus,

$$Inrisk 1 = ( sqrt ( percent\_density ) - sqrt ( {mean}_{DENS} ) ) \times {β}_{DENS} + ln ( {fh\_risk}_{-} 1 ) + ln ( {fh\_risk}_{-} 2 ) + {intercept}_{PRE} + ( ( ln ( bmi / 5 ) - ln ( {mean}_{BMI} / 5 ) ) \times {β}_{PRE} ) if pre ‐ menopausal + {intercept}_{POST} + \text{} ( ( ln ( bmi / 5 ) - ln ( {mean}_{BMI} / 5 ) ) \times {β}_{POST} ) if post ‐ menopausal , \\ or \\ Inrisk 1 = X if BI ‐ RADS category is A or B + Y if BI ‐ RADS category is C or D + ln ( {fh\_risk}_{-} 1 ) + ln ( {fh\_risk}_{-} 2 ) + {intercept}_{PRE} + ( ( ln ( bmi / 5 ) - ln ( {mean}_{BMI} / 5 ) ) \times {β}_{PRE} ) if pre ‐ menopausal + {intercept}_{POST} + ( ( ln ( bmi / 5 ) - ln ( {mean}_{BMI} / 5 ) ) \times {β}_{POST} ) if post ‐ menopausal$$

In a further aspect, the present invention provides a computer readable storage medium storing executable code, wherein when a processor executes the code, the processor is cause to perform the method of the invention.

In another aspect, the present invention provides a device for assessing the risk of a human female subject developing breast cancer, the device comprising:
a processor; and
a memory device storing executable code, the memory being accessible to the processor;
   wherein when caused to execute the executable code stored in the memory device, the processor is caused to:
determine a genetic risk score based on genetic data derived from a biological sample taken from the female subject;
determine a mammographic density risk score based on data derived from a clinical assessment of the mammographic density of the female subject;
determine a first-degree family history risk score and a second-degree family history risk score based on family history data of the female subject;
determine a body mass index risk score based on the body mass index and menopause status of the subject;
calculate a risk modifier score, the risk modifier score being a function of the polygenetic risk score, mammographic density risk score, first-degree family history risk score, second-degree family history risk score and body mass index risk score
calculate at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculate the female subject's absolute risk of breast cancer in the next N years, and/or in the subjects full lifetime, using the cumulative risk scores.

Embodiments of the first aspect are relevant to this aspect.

In another aspect, the present invention provides a device for assessing the risk of a human female subject developing breast cancer, the device comprising:
a processor; and
a memory device storing executable code, the memory being accessible to the processor;
   wherein when caused to execute the executable code stored in the memory device, the processor is caused to:
determine a mammographic density risk score based on data derived from a clinical assessment of the mammographic density of the female subject;
determine a first-degree family history risk score and a second-degree family history risk score based on family history data of the female subject;
determine a body mass index risk score based on the body mass index and menopause status of the subject;
calculate a risk modifier score, the risk modifier score being a function of the mammographic density risk score, first-degree family history risk score, second-degree family history risk score and body mass index risk score
calculate at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculate the female subject's absolute risk of breast cancer in the next N years, and/or in the subjects full lifetime, using the cumulative risk scores.

Embodiments of the second aspect are relevant to this aspect.

In an embodiment, the device further comprises a display component, wherein the processor is further caused to display the breast cancer risk score of the female subject for developing breast cancer on the display component.

In an embodiment, the device further comprises a communications module, wherein the processor is further caused to communicate the breast cancer risk score of the female subject for developing breast cancer to an external device via the communications module.

In an aspect, the present invention provides a method for assessing the risk of a human female subject for developing breast cancer comprising:
i) determining the age, ethnicity, family history of breast cancer incorporating number of first-degree relatives who have had breast cancer, the age of the youngest first-degree relative at diagnosis with breast cancer, the number of second-degree relatives who have had breast cancer, country of residence, mammographic breast density, body mass index (BMI) and menopausal status,
ii) determining the genotype of the human female subject at 50 or more polymorphic loci associated with a risk of a human female subject for developing breast cancer,
iii) determining a breast cancer risk score based on i) and ii).

In an aspect, the present invention provides a method for assessing the risk of a human female subject for developing breast cancer comprising:
i) determining the age, ethnicity, family history of breast cancer incorporating number of first-degree relatives who have had breast cancer, the age of the youngest first-degree relative at diagnosis with breast cancer, the number of second-degree relatives who have had breast cancer, mammographic breast density, body mass index (BMI) and menopausal status,
ii) determining the genotype of the human female subject at 50 or more polymorphic loci associated with a risk of a human female subject for developing breast cancer,
iii) determining a breast cancer risk score based on i) and ii).

Embodiments of the first aspect are relevant to the above two aspects.

In an aspect, the present invention provides a method for assessing the risk of a human female subject for developing breast cancer comprising:
i) determining the age, ethnicity, family history of breast cancer incorporating number of first-degree relatives who have had breast cancer, the age of the youngest first-degree relative at diagnosis with breast cancer, the number of second-degree relatives who have had breast cancer, country of residence, mammographic breast density, body mass index (BMI) and menopausal status,
ii) determining a breast cancer risk score based on i).

In an aspect, the present invention provides a method for assessing the risk of a human female subject for developing breast cancer comprising:
i) determining the age, ethnicity, family history of breast cancer incorporating number of first-degree relatives who have had breast cancer, the age of the youngest first-degree relative at diagnosis with breast cancer, the number of second-degree relatives who have had breast cancer, mammographic breast density, body mass index (BMI) and menopausal status,
ii) determining a breast cancer risk score based on i).

Embodiments of the second aspect are relevant to the above two aspects.

In an embodiment, the results of the assessment indicate that the female should be subjected to more frequent screening and/or prophylactic anti-breast cancer therapy. In an embodiment, if it is determined that the subject has a risk of developing breast cancer, the subject is more likely to be responsive to oestrogen inhibition therapy than non-responsive.

In an embodiment, the breast cancer is oestrogen receptor positive or oestrogen receptor negative.

Also provided is a method for determining the need for routine diagnostic testing of a human female subject for breast cancer comprising assessing the risk of the subject for developing breast cancer using a method of the invention.

In an embodiment, an risk score greater than about 20% lifetime risk indicates that the subject should be enrolled in a screening breast MRI and mammography program.

In an aspect, the present invention provides a method of screening for breast cancer in a human female subject, the method comprising assessing the risk of the subject for developing breast cancer using a method of the invention, and routinely screening for breast cancer in the subject if they are assessed as having a risk for developing breast cancer.

In an aspect, the present invention provides a method for determining the need of a human female subject for prophylactic anti-breast cancer therapy comprising assessing the risk of the subject for developing breast cancer using a method of the invention.

In an embodiment, an risk score greater than about 1.66% 5-year risk indicates that oestrogen receptor antagonist therapy should be offered to the subject.

In an aspect, the present invention provides a method for preventing breast cancer in a human female subject, the method comprising assessing the risk of the subject for developing breast cancer using a method of the invention, and administering an anti-breast cancer therapy to the subject if they are assessed as having a risk for developing breast cancer.

In an embodiment, the therapy inhibits oestrogen.

In an aspect, the present invention provides an anti-breast cancer therapy for use in preventing breast cancer in a human female subject at risk thereof, wherein the subject is assessed as having a risk for developing breast cancer using a method of the invention.

In an aspect, the present invention provides a method for stratifying a group of human female subjects for a clinical trial of a candidate therapy, the method comprising assessing the individual risk of the subjects for developing breast cancer using a method of the invention, and using the results of the assessment to select subjects more likely to be responsive to the therapy.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying FIGURES.

### BRIEF DESCRIPTION OF THE ACCOMPANYING DRAWINGS

FIG. 1. Calibration plots of expected versus observed odds ratio (OR) of the respective risk models.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., oncology, breast cancer analysis, molecular genetics, risk assessment and clinical studies).

Unless otherwise indicated, the molecular, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

It is to be understood that this disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule.

As used herein, the term “about”, unless stated to the contrary, refers to +/−10%, more preferably +/−5%, more preferably +/−1%, of the designated value.

The methods of the present disclosure can be used to assess risk of a human female subject developing breast cancer. As used herein, the term “breast cancer” encompasses any type of breast cancer that can develop in a female subject. For example, the breast cancer may be characterised as Luminal A (ER+ and/or PR+, HER2−, low Ki67), Luminal B (ER+ and/or PR+, HER2+(or HER2− with high Ki67), Triple negative/basal-like (ER−, PR−, HER2−) or HER2 type (ER−, PR−, HER2+). In another example, the breast cancer may be resistant to therapy or therapies such as alkylating agents, platinum agents, taxanes, vinca agents, anti-estrogen drugs, aromatase inhibitors, ovarian suppression agents, endocrine/hormonal agents, bisphophonate therapy agents or targeted biological therapy agents. As used herein, “breast cancer” also encompasses a phenotype that displays a predisposition towards developing breast cancer in an individual. A phenotype that displays a predisposition for breast cancer, can, for example, show a higher likelihood that the cancer will develop in an individual with the phenotype than in members of a relevant general population under a given set of environmental conditions (diet, physical activity regime, geographic location, etc.).

As used herein, “biological sample” refers to any sample comprising nucleic acids, especially DNA, from or derived from a human patient, e.g., bodily fluids (blood, saliva, urine etc.), biopsy, tissue, and/or waste from the patient. Thus, tissue biopsies, stool, sputum, saliva, blood, lymph, or the like can easily be screened for polymorphisms, as can essentially any tissue of interest that contains the appropriate nucleic acids. In one embodiment, the biological sample is a cheek cell sample. These samples are typically taken, following informed consent, from a patient by standard medical laboratory methods. The sample may be in a form taken directly from the patient, or may be at least partially processed (purified) to remove at least some non-nucleic acid material.

A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations). Other examples include a deletion or insertion of one or more base pairs at the polymorphism locus.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele “negatively” correlates with a trait when it is linked to it and when presence of the allele is an indicator that a trait or trait form will not occur in an individual comprising the allele.

A marker polymorphism or allele is “correlated” or “associated” with a specified phenotype (breast cancer susceptibility, etc.) when it can be statistically linked (positively or negatively) to the phenotype. Methods for determining whether a polymorphism or allele is statistically linked are known to those in the art. That is, the specified polymorphism occurs more commonly in a case population (e.g., breast cancer patients) than in a control population (e.g., individuals that do not have breast cancer). This correlation is often inferred as being causal in nature, but it need not be, simple genetic linkage to (association with) a locus for a trait that underlies the phenotype is sufficient for correlation/association to occur.

The phrase “linkage disequilibrium” (LD) is used to describe the statistical correlation between two neighbouring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) (Devlin and Risch, 1995). Two loci with a LD value of 1 are said to be in complete LD. At the other extreme, two loci with a LD value of 0 are termed to be in linkage equilibrium. Linkage disequilibrium is calculated following the application of the expectation maximization algorithm (EM) for the estimation of haplotype frequencies (Slatkin and Excoffier, 1996). LD values according to the present disclosure for neighbouring genotypes/loci are selected above 0.1, preferably, above 0.2, more preferable above 0.5, more preferably, above 0.6, still more preferably, above 0.7, preferably, above 0.8, more preferably above 0.9, ideally about 1.0.

Another way one of skill in the art can readily identify polymorphisms in linkage disequilibrium with the polymorphisms of the present disclosure is determining the LOD score for two loci. LOD stands for “logarithm of the odds”, a statistical estimate of whether two genes, or a gene and a disease gene, are likely to be located near each other on a chromosome and are therefore likely to be inherited. A LOD score of between about 2-3 or higher is generally understood to mean that two genes are located close to each other on the chromosome. The present inventors have found that many of the polymorphisms in linkage disequilibrium with the polymorphisms of the present disclosure have a LOD score of between about 2-50. Accordingly, in an embodiment, LOD values according to the present disclosure for neighbouring genotypes/loci are selected at least above 2, at least above 3, at least above 4, at least above 5, at least above 6, at least above 7, at least above 8, at least above 9, at least above 10, at least above 20 at least above 30, at least above 40, at least above 50.

In another embodiment, polymorphisms in linkage disequilibrium with the polymorphisms of the present disclosure can have a specified genetic recombination distance of less than or equal to about 20 centimorgan (cM) or less. For example, 15 cM or less, 10 cM or less, 9 cM or less, 8 cM or less, 7 cM or less, 6 cM or less, 5 CM or less, 4 cM or less, 3 cM or less, 2 cM or less, 1 cM or less, 0.75 cM or less, 0.5 cM or less, 0.25 cM or less, or 0.1 cM or less. For example, two linked loci within a single chromosome segment can undergo recombination during meiosis with each other at a frequency of less than or equal to about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.1% or less.

In another embodiment, polymorphisms in linkage disequilibrium with the polymorphisms of the present disclosure are within at least 100 kb (which correlates in humans to about 0.1 cM, depending on local recombination rate), at least 50 kb, at least kb or less of each other.

For example, one approach for the identification of surrogate markers for a particular polymorphism involves a simple strategy that presumes that polymorphisms surrounding the target polymorphism are in linkage disequilibrium and can therefore provide information about disease susceptibility. Thus, as described herein, surrogate markers can therefore be identified from publicly available databases, such as HAPMAP, by searching for polymorphisms fulfilling certain criteria which have been found in the scientific community to be suitable for the selection of surrogate marker candidates.

“Allele frequency” refers to the frequency (proportion or percentage) at which an allele is present at a locus within an individual, within a line or within a population of lines. For example, for an allele “A,” diploid individuals of genotype “AA”, “Aa” or “aa” have allele frequencies of 1.0, 0.5, or 0.0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

In an embodiment, the term “allele frequency” is used to define the minor allele frequency (MAF) or effect allele frequency. MAF refers to the frequency at which the least common allele occurs in a given population. The effect allele is that linked to breast cancer risk.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

A “marker”, “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, nRNA, mRNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Each of the identified markers is expected to be in close physical and genetic proximity (resulting in physical and/or genetic linkage) to a genetic element, e.g., a QTL, that contributes to the relevant phenotype. Markers corresponding to genetic polymorphisms between members of a population can be detected by methods well-established in the art. These include, e.g., DNA sequencing, PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “genotype” is the genetic constitution of an individual (or group of individuals) at one or more genetic loci. Genotype is defined by the allele(s) of one or more known loci of the individual, typically, the compilation of alleles inherited from its parents.

A “haplotype” is the genotype of an individual at a plurality of genetic loci on a single DNA strand. Typically, the genetic loci described by a haplotype are physically and genetically linked, i.e., on the same chromosome strand.

A “set” of markers, probes or primers refers to a collection or group of markers probes, primers, or the data derived therefrom, used for a common purpose, e.g., identifying an individual with a specified genotype (e.g., risk of developing breast cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

The polymorphisms and genes, and corresponding marker probes, amplicons or primers described above can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods above, the set of marker probes or primers optionally detects a plurality of polymorphisms in a plurality of said genes or genetic loci. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

As used herein, “risk assessment” refers to a process by which a subject's risk of developing breast cancer can be assessed. A risk assessment will typically involve obtaining information relevant to the subject's risk of developing breast cancer, assessing that information, and quantifying the subject's risk of developing breast cancer, for example, by producing a risk score.

As used herein, “receiver operating characteristic curves” (ROC) refer to a graphical plot of the sensitivity vs. (1−specificity) for a binary classifier system as its discrimination threshold is varied. The ROC can also be represented equivalently by plotting the fraction of true positives (TPR=true positive rate) vs. the fraction of false positives (FPR=false positive rate). Also known as a Relative Operating Characteristic curve, because it is a comparison of two operating characteristics (TPR & FPR) as the criterion changes. ROC analysis provides tools to select possibly optimal models and to discard suboptimal ones independently from (and prior to specifying) the cost context or the class distribution. Methods of using in the context of the disclosure will be clear to those skilled in the art.

As used herein, the terms “routinely screening for breast cancer” and “more frequent screening” are relative terms, and are based on a comparison to the level of screening recommended to a subject who has no identified risk of developing breast cancer.

### Clinical Risk Assessments

The present invention comprises, preferably consists of, the analysis of the following clinical factors; age, ethnicity, family history of breast cancer (incorporating number of first-degree relatives who have had breast cancer, the youngest age of first-degree relative at diagnosis with breast cancer, the number of second degree relatives who have had breast cancer), country of residence, mammographic breast density (for example as a BI-RADS score or percentage breast density), body mass index (BMI) and menopausal status.

Clinical information can be self-reported by the female subject. For example, the subject may complete a questionnaire designed to obtain clinical information regarding the above factors. In another example, subject to obtaining informed consent from the female subject, clinical information can be obtained from medical records by interrogating a relevant database comprising the clinical information.

In an embodiment, the clinical risk assessment procedure provides an estimate of the risk of the human female subject developing breast cancer during the next N-year period (for example 5-year risk).

In another embodiment, the clinical risk assessment procedure provides an estimate of the risk of the human female subject developing breast cancer from her current age up to age 90 years (i.e. remaining lifetime risk).

In another embodiment, performing the clinical risk assessment uses a model which calculates the absolute risk of developing breast cancer. For example, the absolute risk of developing breast cancer can be calculated using cancer incidence rates.

In an embodiment, the clinical risk assessment provides a 5-year absolute risk of developing breast cancer. In another embodiment, the clinical risk assessment provides a 10-year absolute risk of developing breast cancer.

“Family history of breast cancer” is used in the context of the present disclosure to refer to the history of breast cancer amongst the female subject's first and/or second degree relatives. For example, “family history of breast cancer” can be used to refer to the history of breast cancer amongst only first-degree relatives. Put another way, the first clinical risk assessment procedure can take into consideration the female subjects family history of breast cancer amongst first-degree relatives. In the context of the present disclosure, a “first-degree relative” is a family member who shares about 50 percent of their genes with the female subject. Examples of first-degree relatives include parents, offspring, and full-siblings. A “second degree relative” is a family member who shares about 25 percent of their genes with the female subject. Examples of second-degree relatives include uncles, aunts, nephews, nieces, grandparents, grandchildren, and half-siblings.

In another embodiment, the female subject's family history of breast cancer is based on the female subject's first degree-relatives and second degree relatives.

In an embodiment, the subjects first-degree relative risk (fh_risk_1) is based on age and the number and age of first-degree relatives with invasive breast cancer. In an embodiment, fh_risk_1 is determined as outlined in Table 1.

TABLE 1
Look up table for determining fh_risk_1.

| Number of | | | |
|---|---|---|---|
| affected first- | Age | Age of affected first- | |
| degree relatives | (yrs) | degree relative (yrs) | fh_risk_1 |
| 0 | All ages | N/A | 0.94 |
| 1 | <40 | <40 | 4.90 |
| 1 | <40 | 40-49 | 2.90 |
| 1 | <40 | 50-59 | 2.60 |
| 1 | <40 | ≥60 | 1.90 |
| 1 | 40-49 | <40 | 2.94 |
| 1 | 40-49 | 40-49 | 2.34 |
| 1 | 40-49 | 50-59 | 2.14 |
| 1 | 40-49 | ≥60 | 1.64 |
| 1 | 50-59 | <40 | 2.05 |
| 1 | 50-59 | 40-49 | 1.85 |
| 1 | 50-59 | 50-59 | 1.55 |
| 1 | 50-59 | ≥60 | 1.55 |
| 1 | ≥60 | <40 | 1.37 |
| 1 | ≥60 | 40-49 | 1.37 |
| 1 | ≥60 | 50-59 | 1.37 |
| 1 | ≥60 | ≥60 | 1.37 |
| 1 | <35 | Unknown | 2.84 |
| 1 | 35-39 | Unknown | 2.43 |
| 1 | 40-44 | Unknown | 2.00 |
| 1 | 45-49 | Unknown | 1.94 |
| 1 | 50-54 | Unknown | 1.84 |
| 1 | 55-59 | Unknown | 1.74 |
| 1 | 60-64 | Unknown | 1.55 |
| 1 | 65-69 | Unknown | 1.46 |
| 1 | ≥70 | Unknown | 1.37 |
| ≥2 | <50 | At least one <40 | 13.50 |
| ≥2 | <50 | Both ≥40 | 7.80 |
| ≥2 | ≥50 | At least one <40 | 3.90 |
| ≥2 | ≥50 | Both ≥40 | 2.60 |
| ≥2 | <45 | Unknown | 5.00 |
| ≥2 | 45-54 | Unknown | 3.50 |
| ≥2 | 55-64 | Unknown | 2.70 |
| ≥2 | ≥65 | Unknown | 2.30 |

In an embodiment, the subjects second-degree relative risk (fh_risk_2) is based on age and the number and age of first-degree relatives with invasive breast cancer. In an embodiment, fh_risk_1 is determined as outlined in Table 2.

TABLE 2
Look up table for determining fh_risk_2.

| Number of affected | Age | |
|---|---|---|
| second-degree relatives | (years) | fh_risk_2 |
| 0 | All ages | 0.97 |
| 1 | <40 | 1.7 |
| 1 | 40-49 | 1.4 |
| 1 | 50-59 | 1.3 |
| 1 | ≥60 | 1.2 |
| ≥2 | <55 | 2.1 |
| ≥2 | ≥55 | 1.6 |

As the skilled person would appreciate, the fh_risk_1 and fh_risk_2 values in Table 1 and 2 can be modified without necessarily affecting the performance of the method. Thus, in an embodiment, one or more or all of the fh_risk_1 and fh_risk_2 values can be varied +/−10%, more preferably +/−5%, more preferably +/−1%, of the designated value on a relative basis.

As used herein, “based on” means that values are assigned to, for example, the subject's age and family history of breast cancer, but then any suitable calculations are conducted to determine clinical risk.

Breast density can be measured using any method known in the art. For example, breast density can be estimated based on radiographic appearance of the breast on a mammogram. As will be known to those skilled in the art, dense breast tissue appears light on a mammogram and comprises epithelial and stromal tissue whereas non-dense tissue, comprising fat, appears dark. Thus, in some embodiments, breast density is assessed using a mammogram.

In an embodiment, breast density is assessed using higher pixel brightness thresholds.

In an embodiment, breast density is assessed using percent dense area. Percent dense area is calculated by dividing the area of dense breast tissue by the total breast area identified in a breast image, for example in a mammogram.

In an embodiment, breast density is assessed using Cumulus percent dense area. In another embodiment breast density is assessed using Cumulus percent dense area and non-dense area. “Cumulus” is a software package for semi-automated measurement of dense area from mammograms and is described in (Byng et al., 1994).

In an embodiment, breast density is assessed using a BI-RADS score. “BI-RADS” is an acronym for Breast Imaging-Reporting and Data System, which is a system of standardized numerical codes typically assigned by a radiologist after interpreting a mammogram and is used to communicate a subject's risk of developing breast cancer. A BI-RADS score can also be obtained using automated computerised methods. Typical BI-RADS Assessment Categories (BI-RADS Atlas) are:
0: Incomplete;
1: Negative;
2: Benign;
3: Probably benign;
4: Suspicious;
5: Highly suggestive of malignancy; and
6: Known biopsy-proven malignancy.

In an embodiment, BI-RADS is scored as an A, B, C or D using the following criteria as described by Sickles and Bassett (2013):
A: Almost entirely fatty indicates that the breasts are almost entirely composed of fat. About 1 in 10 women has this result.
B: Scattered areas of fibroglandular density indicates there are some scattered areas of density, but the majority of the breast tissue is nondense. About 4 in 10 women have this result.
C: Heterogeneously dense indicates that there are some areas of nondense tissue, but that the majority of the breast tissue is dense. About 4 in 10 women have this result.
D: Extremely dense indicates that nearly all of the breast tissue is dense. About 1 in 10 women has this result.

Body mass index (BMI) is a value derived from the mass (weight) and height of a person. The BMI is defined as the body mass divided by the square of the body height, and is expressed in units of kg/m², resulting from mass in kilograms and height in metres.

Menopausal status is typically determined if it has been over a year since the subjects last period. In an embodiment, the subject is scored as a 1 if it has been over a year since the subjects last period and otherwise scored as 0.

In an embodiment, ethnicity is taken into consideration by using ethnicity-specific population-based breast cancer incidence data. This is derived from publically available country- and ethnicity-specific population-based breast cancer incidence data. For example, in Tables 3 to 7, incid is the breast cancer incidence per 100,000 women per year at a given age, incid_b is the sum of the breast cancer incidence from birth to the given age; incid_b_N is the sum of the breast cancer incidence from birth to the given age plus N years and incid_full_life is the sum of the breast cancer incidence from birth to age 90 years, for different populations and ethnicities. Incidence data can be obtained from the relevant National agencies, for example, the Office of National Statistics in England (www.ons.gov.uk), the Surveillance, Epidemiology, and End Results Program in the United States (seer.cancer.gov) and the Australian Institute of Health and Welfare in Australia (www.aihw.gov.au).

TABLE 3
Look up table for Caucasian United Kingdom specific
population-based breast cancer incidence data.

| Age | Incidence | incid | incid_b | incid_b_5 | incid_b_10 | incid_full_life |
|---|---|---|---|---|---|---|
| 0 | 0.0 | 0.000000 | 0.000000 | 0.000000 | 0.000000 | 0.172625 |
| 1 | 0.0 | 0.000000 | 0.000000 | 0.000000 | 0.000000 | 0.172625 |
| 2 | 0.0 | 0.000000 | 0.000000 | 0.000000 | 0.000000 | 0.172625 |
| 3 | 0.0 | 0.000000 | 0.000000 | 0.000000 | 0.000000 | 0.172625 |
| 4 | 0.0 | 0.000000 | 0.000000 | 0.000000 | 0.000000 | 0.172625 |
| 5 | 0.0 | 0.000000 | 0.000000 | 0.000000 | 0.000000 | 0.172625 |
| 6 | 0.0 | 0.000000 | 0.000000 | 0.000000 | 0.000000 | 0.172625 |
| 7 | 0.0 | 0.000000 | 0.000000 | 0.000000 | 0.000000 | 0.172625 |
| 8 | 0.0 | 0.000000 | 0.000000 | 0.000000 | 0.000000 | 0.172625 |
| 9 | 0.0 | 0.000000 | 0.000000 | 0.000000 | 0.000000 | 0.172625 |
| 10 | 0.0 | 0.000000 | 0.000000 | 0.000000 | 0.000015 | 0.172625 |
| 11 | 0.0 | 0.000000 | 0.000000 | 0.000000 | 0.000030 | 0.172625 |
| 12 | 0.0 | 0.000000 | 0.000000 | 0.000000 | 0.000045 | 0.172625 |
| 13 | 0.0 | 0.000000 | 0.000000 | 0.000000 | 0.000060 | 0.172625 |
| 14 | 0.0 | 0.000000 | 0.000000 | 0.000000 | 0.000075 | 0.172625 |
| 15 | 0.0 | 0.000000 | 0.000000 | 0.000015 | 0.000181 | 0.172625 |
| 16 | 0.0 | 0.000000 | 0.000000 | 0.000030 | 0.000287 | 0.172625 |
| 17 | 0.0 | 0.000000 | 0.000000 | 0.000045 | 0.000393 | 0.172625 |
| 18 | 0.0 | 0.000000 | 0.000000 | 0.000060 | 0.000499 | 0.172625 |
| 19 | 0.0 | 0.000000 | 0.000000 | 0.000075 | 0.000605 | 0.172625 |
| 20 | 1.5 | 0.000015 | 0.000015 | 0.000181 | 0.000924 | 0.172625 |
| 21 | 1.5 | 0.000015 | 0.000030 | 0.000287 | 0.001243 | 0.172625 |
| 22 | 1.5 | 0.000015 | 0.000045 | 0.000393 | 0.001562 | 0.172625 |
| 23 | 1.5 | 0.000015 | 0.000060 | 0.000499 | 0.001881 | 0.172625 |
| 24 | 1.5 | 0.000015 | 0.000075 | 0.000605 | 0.002200 | 0.172625 |
| 25 | 10.6 | 0.000106 | 0.000181 | 0.000924 | 0.002868 | 0.172625 |
| 26 | 10.6 | 0.000106 | 0.000287 | 0.001243 | 0.003536 | 0.172625 |
| 27 | 10.6 | 0.000106 | 0.000393 | 0.001562 | 0.004204 | 0.172625 |
| 28 | 10.6 | 0.000106 | 0.000499 | 0.001881 | 0.004872 | 0.172625 |
| 29 | 10.6 | 0.000106 | 0.000605 | 0.002200 | 0.005540 | 0.172625 |
| 30 | 31.9 | 0.000319 | 0.000924 | 0.002868 | 0.006794 | 0.172625 |
| 31 | 31.9 | 0.000319 | 0.001243 | 0.003536 | 0.008048 | 0.172625 |
| 32 | 31.9 | 0.000319 | 0.001562 | 0.004204 | 0.009302 | 0.172625 |
| 33 | 31.9 | 0.000319 | 0.001881 | 0.004872 | 0.010556 | 0.172625 |
| 34 | 31.9 | 0.000319 | 0.002200 | 0.005540 | 0.011810 | 0.172625 |
| 35 | 66.8 | 0.000668 | 0.002868 | 0.006794 | 0.013972 | 0.172625 |
| 36 | 66.8 | 0.000668 | 0.003536 | 0.008048 | 0.016134 | 0.172625 |
| 37 | 66.8 | 0.000668 | 0.004204 | 0.009302 | 0.018296 | 0.172625 |
| 38 | 66.8 | 0.000668 | 0.004872 | 0.010556 | 0.020458 | 0.172625 |
| 39 | 66.8 | 0.000668 | 0.005540 | 0.011810 | 0.022620 | 0.172625 |
| 40 | 125.4 | 0.001254 | 0.006794 | 0.013972 | 0.025364 | 0.172625 |
| 41 | 125.4 | 0.001254 | 0.008048 | 0.016134 | 0.028108 | 0.172625 |
| 42 | 125.4 | 0.001254 | 0.009302 | 0.018296 | 0.030852 | 0.172625 |
| 43 | 125.4 | 0.001254 | 0.010556 | 0.020458 | 0.033596 | 0.172625 |
| 44 | 125.4 | 0.001254 | 0.011810 | 0.022620 | 0.036340 | 0.172625 |
| 45 | 216.2 | 0.002162 | 0.013972 | 0.025364 | 0.039212 | 0.172625 |
| 46 | 216.2 | 0.002162 | 0.016134 | 0.028108 | 0.042084 | 0.172625 |
| 47 | 216.2 | 0.002162 | 0.018296 | 0.030852 | 0.044956 | 0.172625 |
| 48 | 216.2 | 0.002162 | 0.020458 | 0.033596 | 0.047828 | 0.172625 |
| 49 | 216.2 | 0.002162 | 0.022620 | 0.036340 | 0.050700 | 0.172625 |
| 50 | 274.4 | 0.002744 | 0.025364 | 0.039212 | 0.053966 | 0.172625 |
| 51 | 274.4 | 0.002744 | 0.028108 | 0.042084 | 0.057232 | 0.172625 |
| 52 | 274.4 | 0.002744 | 0.030852 | 0.044956 | 0.060498 | 0.172625 |
| 53 | 274.4 | 0.002744 | 0.033596 | 0.047828 | 0.063764 | 0.172625 |
| 54 | 274.4 | 0.002744 | 0.036340 | 0.050700 | 0.067030 | 0.172625 |
| 55 | 287.2 | 0.002872 | 0.039212 | 0.053966 | 0.071126 | 0.172625 |
| 56 | 287.2 | 0.002872 | 0.042084 | 0.057232 | 0.075222 | 0.172625 |
| 57 | 287.2 | 0.002872 | 0.044956 | 0.060498 | 0.079318 | 0.172625 |
| 58 | 287.2 | 0.002872 | 0.047828 | 0.063764 | 0.083414 | 0.172625 |
| 59 | 287.2 | 0.002872 | 0.050700 | 0.067030 | 0.087510 | 0.172625 |
| 60 | 326.6 | 0.003266 | 0.053966 | 0.071126 | 0.091148 | 0.172625 |
| 61 | 326.6 | 0.003266 | 0.057232 | 0.075222 | 0.094786 | 0.172625 |
| 62 | 326.6 | 0.003266 | 0.060498 | 0.079318 | 0.098424 | 0.172625 |
| 63 | 326.6 | 0.003266 | 0.063764 | 0.083414 | 0.102062 | 0.172625 |
| 64 | 326.6 | 0.003266 | 0.067030 | 0.087510 | 0.105700 | 0.172625 |
| 65 | 409.6 | 0.004096 | 0.071126 | 0.091148 | 0.109556 | 0.172625 |
| 66 | 409.6 | 0.004096 | 0.075222 | 0.094786 | 0.113412 | 0.172625 |
| 67 | 409.6 | 0.004096 | 0.079318 | 0.098424 | 0.117268 | 0.172625 |
| 68 | 409.6 | 0.004096 | 0.083414 | 0.102062 | 0.121124 | 0.172625 |
| 69 | 409.6 | 0.004096 | 0.087510 | 0.105700 | 0.124980 | 0.172625 |
| 70 | 363.8 | 0.003638 | 0.091148 | 0.109556 | 0.129197 | 0.172625 |
| 71 | 363.8 | 0.003638 | 0.094786 | 0.113412 | 0.133414 | 0.172625 |
| 72 | 363.8 | 0.003638 | 0.098424 | 0.117268 | 0.137631 | 0.172625 |
| 73 | 363.8 | 0.003638 | 0.102062 | 0.121124 | 0.141848 | 0.172625 |
| 74 | 363.8 | 0.003638 | 0.105700 | 0.124980 | 0.146065 | 0.172625 |
| 75 | 385.6 | 0.003856 | 0.109556 | 0.129197 | 0.150475 | 0.172625 |
| 76 | 385.6 | 0.003856 | 0.113412 | 0.133414 | 0.154885 | 0.172625 |
| 77 | 385.6 | 0.003856 | 0.117268 | 0.137631 | 0.159295 | 0.172625 |
| 78 | 385.6 | 0.003856 | 0.121124 | 0.141848 | 0.163705 | 0.172625 |
| 79 | 385.6 | 0.003856 | 0.124980 | 0.146065 | 0.168115 | 0.172625 |
| 80 | 421.7 | 0.004217 | 0.129197 | 0.150475 | 0.172625 | 0.172625 |
| 81 | 421.7 | 0.004217 | 0.133414 | 0.154885 | | 0.172625 |
| 82 | 421.7 | 0.004217 | 0.137631 | 0.159295 | | 0.172625 |
| 83 | 421.7 | 0.004217 | 0.141848 | 0.163705 | | 0.172625 |
| 84 | 421.7 | 0.004217 | 0.146065 | 0.168115 | | 0.172625 |
| 85 | 441.0 | 0.004410 | 0.150475 | 0.172625 | | 0.172625 |
| 86 | 441.0 | 0.004410 | | | | 0.172625 |
| 87 | 441.0 | 0.004410 | | | | 0.172625 |
| 88 | 441.0 | 0.004410 | | | | 0.172625 |
| 89 | 441.0 | 0.004410 | | | | 0.172625 |
| 90 | 451.0 | 0.004510 | | | | 0.172625 |

TABLE 4
Look up table for Caucasian Australian specific
population-based breast cancer incidence data.

| Age | Incidence | incid | incid_b | incid_b_5 | incid_b_10 | incid_full_life |
|---|---|---|---|---|---|---|
| 0 | 0.0 | 0.000000 | 0.000000 | 0.000000 | 0.000000 | 0.159749 |
| 1 | 0.0 | 0.000000 | 0.000000 | 0.000000 | 0.000000 | 0.159749 |
| 2 | 0.0 | 0.000000 | 0.000000 | 0.000000 | 0.000000 | 0.159749 |
| 3 | 0.0 | 0.000000 | 0.000000 | 0.000000 | 0.000000 | 0.159749 |
| 4 | 0.0 | 0.000000 | 0.000000 | 0.000000 | 0.000000 | 0.159749 |
| 5 | 0.0 | 0.000000 | 0.000000 | 0.000000 | 0.000003 | 0.159749 |
| 6 | 0.0 | 0.000000 | 0.000000 | 0.000000 | 0.000006 | 0.159749 |
| 7 | 0.0 | 0.000000 | 0.000000 | 0.000000 | 0.000010 | 0.159749 |
| 8 | 0.0 | 0.000000 | 0.000000 | 0.000000 | 0.000013 | 0.159749 |
| 9 | 0.0 | 0.000000 | 0.000000 | 0.000000 | 0.000016 | 0.159749 |
| 10 | 0.0 | 0.000000 | 0.000000 | 0.000003 | 0.000026 | 0.159749 |
| 11 | 0.0 | 0.000000 | 0.000000 | 0.000006 | 0.000035 | 0.159749 |
| 12 | 0.0 | 0.000000 | 0.000000 | 0.000010 | 0.000044 | 0.159749 |
| 13 | 0.0 | 0.000000 | 0.000000 | 0.000013 | 0.000054 | 0.159749 |
| 14 | 0.0 | 0.000000 | 0.000000 | 0.000016 | 0.000063 | 0.159749 |
| 15 | 0.3 | 0.000003 | 0.000003 | 0.000026 | 0.000154 | 0.159749 |
| 16 | 0.3 | 0.000003 | 0.000006 | 0.000035 | 0.000244 | 0.159749 |
| 17 | 0.3 | 0.000003 | 0.000010 | 0.000044 | 0.000335 | 0.159749 |
| 18 | 0.3 | 0.000003 | 0.000013 | 0.000054 | 0.000425 | 0.159749 |
| 19 | 0.3 | 0.000003 | 0.000016 | 0.000063 | 0.000516 | 0.159749 |
| 20 | 0.9 | 0.000009 | 0.000026 | 0.000154 | 0.000772 | 0.159749 |
| 21 | 0.9 | 0.000009 | 0.000035 | 0.000244 | 0.001027 | 0.159749 |
| 22 | 0.9 | 0.000009 | 0.000044 | 0.000335 | 0.001283 | 0.159749 |
| 23 | 0.9 | 0.000009 | 0.000054 | 0.000425 | 0.001539 | 0.159749 |
| 24 | 0.9 | 0.000009 | 0.000063 | 0.000516 | 0.001795 | 0.159749 |
| 25 | 9.1 | 0.000091 | 0.000154 | 0.000772 | 0.002395 | 0.159749 |
| 26 | 9.1 | 0.000091 | 0.000244 | 0.001027 | 0.002994 | 0.159749 |
| 27 | 9.1 | 0.000091 | 0.000335 | 0.001283 | 0.003594 | 0.159749 |
| 28 | 9.1 | 0.000091 | 0.000425 | 0.001539 | 0.004193 | 0.159749 |
| 29 | 9.1 | 0.000091 | 0.000516 | 0.001795 | 0.004793 | 0.159749 |
| 30 | 25.6 | 0.000256 | 0.000772 | 0.002395 | 0.006065 | 0.159749 |
| 31 | 25.6 | 0.000256 | 0.001027 | 0.002994 | 0.007338 | 0.159749 |
| 32 | 25.6 | 0.000256 | 0.001283 | 0.003594 | 0.008610 | 0.159749 |
| 33 | 25.6 | 0.000256 | 0.001539 | 0.004193 | 0.009882 | 0.159749 |
| 34 | 25.6 | 0.000256 | 0.001795 | 0.004793 | 0.011155 | 0.159749 |
| 35 | 60.0 | 0.000600 | 0.002395 | 0.006065 | 0.013250 | 0.159749 |
| 36 | 60.0 | 0.000600 | 0.002994 | 0.007338 | 0.015345 | 0.159749 |
| 37 | 60.0 | 0.000600 | 0.003594 | 0.008610 | 0.017440 | 0.159749 |
| 38 | 60.0 | 0.000600 | 0.004193 | 0.009882 | 0.019535 | 0.159749 |
| 39 | 60.0 | 0.000600 | 0.004793 | 0.011155 | 0.021630 | 0.159749 |
| 40 | 127.2 | 0.001272 | 0.006065 | 0.013250 | 0.024190 | 0.159749 |
| 41 | 127.2 | 0.001272 | 0.007338 | 0.015345 | 0.026749 | 0.159749 |
| 42 | 127.2 | 0.001272 | 0.008610 | 0.017440 | 0.029309 | 0.159749 |
| 43 | 127.2 | 0.001272 | 0.009882 | 0.019535 | 0.031868 | 0.159749 |
| 44 | 127.2 | 0.001272 | 0.011155 | 0.021630 | 0.034428 | 0.159749 |
| 45 | 209.5 | 0.002095 | 0.013250 | 0.024190 | 0.037170 | 0.159749 |
| 46 | 209.5 | 0.002095 | 0.015345 | 0.026749 | 0.039912 | 0.159749 |
| 47 | 209.5 | 0.002095 | 0.017440 | 0.029309 | 0.042654 | 0.159749 |
| 48 | 209.5 | 0.002095 | 0.019535 | 0.031868 | 0.045396 | 0.159749 |
| 49 | 209.5 | 0.002095 | 0.021630 | 0.034428 | 0.048138 | 0.159749 |
| 50 | 255.9 | 0.002559 | 0.024190 | 0.037170 | 0.051618 | 0.159749 |
| 51 | 255.9 | 0.002559 | 0.026749 | 0.039912 | 0.055099 | 0.159749 |
| 52 | 255.9 | 0.002559 | 0.029309 | 0.042654 | 0.058580 | 0.159749 |
| 53 | 255.9 | 0.002559 | 0.031868 | 0.045396 | 0.062060 | 0.159749 |
| 54 | 255.9 | 0.002559 | 0.034428 | 0.048138 | 0.065541 | 0.159749 |
| 55 | 274.2 | 0.002742 | 0.037170 | 0.051618 | 0.069581 | 0.159749 |
| 56 | 274.2 | 0.002742 | 0.039912 | 0.055099 | 0.073621 | 0.159749 |
| 57 | 274.2 | 0.002742 | 0.042654 | 0.058580 | 0.077661 | 0.159749 |
| 58 | 274.2 | 0.002742 | 0.045396 | 0.062060 | 0.081701 | 0.159749 |
| 59 | 274.2 | 0.002742 | 0.048138 | 0.065541 | 0.085741 | 0.159749 |
| 60 | 348.1 | 0.003481 | 0.051618 | 0.069581 | 0.089865 | 0.159749 |
| 61 | 348.1 | 0.003481 | 0.055099 | 0.073621 | 0.093989 | 0.159749 |
| 62 | 348.1 | 0.003481 | 0.058580 | 0.077661 | 0.098113 | 0.159749 |
| 63 | 348.1 | 0.003481 | 0.062060 | 0.081701 | 0.102237 | 0.159749 |
| 64 | 348.1 | 0.003481 | 0.065541 | 0.085741 | 0.106361 | 0.159749 |
| 65 | 404.0 | 0.004040 | 0.069581 | 0.089865 | 0.109943 | 0.159749 |
| 66 | 404.0 | 0.004040 | 0.073621 | 0.093989 | 0.113526 | 0.159749 |
| 67 | 404.0 | 0.004040 | 0.077661 | 0.098113 | 0.117109 | 0.159749 |
| 68 | 404.0 | 0.004040 | 0.081701 | 0.102237 | 0.120692 | 0.159749 |
| 69 | 404.0 | 0.004040 | 0.085741 | 0.106361 | 0.124275 | 0.159749 |
| 70 | 412.4 | 0.004124 | 0.089865 | 0.109943 | 0.127558 | 0.159749 |
| 71 | 412.4 | 0.004124 | 0.093989 | 0.113526 | 0.130842 | 0.159749 |
| 72 | 412.4 | 0.004124 | 0.098113 | 0.117109 | 0.134125 | 0.159749 |
| 73 | 412.4 | 0.004124 | 0.102237 | 0.120692 | 0.137409 | 0.159749 |
| 74 | 412.4 | 0.004124 | 0.106361 | 0.124275 | 0.140693 | 0.159749 |
| 75 | 358.3 | 0.003583 | 0.109943 | 0.127558 | 0.143869 | 0.159749 |
| 76 | 358.3 | 0.003583 | 0.113526 | 0.130842 | 0.147045 | 0.159749 |
| 77 | 358.3 | 0.003583 | 0.117109 | 0.134125 | 0.150221 | 0.159749 |
| 78 | 358.3 | 0.003583 | 0.120692 | 0.137409 | 0.153397 | 0.159749 |
| 79 | 358.3 | 0.003583 | 0.124275 | 0.140693 | 0.156573 | 0.159749 |
| 80 | 328.4 | 0.003284 | 0.127558 | 0.143869 | 0.159749 | 0.159749 |
| 81 | 328.4 | 0.003284 | 0.130842 | 0.147045 | | 0.159749 |
| 82 | 328.4 | 0.003284 | 0.134125 | 0.150221 | | 0.159749 |
| 83 | 328.4 | 0.003284 | 0.137409 | 0.153397 | | 0.159749 |
| 84 | 328.4 | 0.003284 | 0.140693 | 0.156573 | | 0.159749 |
| 85 | 317.6 | 0.003176 | 0.143869 | 0.159749 | | 0.159749 |
| 86 | 317.6 | 0.003176 | | | | 0.159749 |
| 87 | 317.6 | 0.003176 | | | | 0.159749 |
| 88 | 317.6 | 0.003176 | | | | 0.159749 |
| 89 | 317.6 | 0.003176 | | | | 0.159749 |
| 90 | 317.6 | 0.003176 | | | | 0.159749 |

TABLE 5
Look up table for Caucasian United States specific
population-based breast cancer incidence data.

| Age | incidence | incid | incid_b | incid_b_5 | incid_b_10 | -incid_life |
|---|---|---|---|---|---|---|
| 0 | 0.0000 | 0.0000 | 0.0000 | 0.0000 | 0.0000 | 0.1850 |
| 1 | 0.0000 | 0.0000 | 0.0000 | 0.0000 | 0.0000 | 0.1850 |
| 2 | 0.0000 | 0.0000 | 0.0000 | 0.0000 | 0.0000 | 0.1850 |
| 3 | 0.0000 | 0.0000 | 0.0000 | 0.0000 | 0.0000 | 0.1850 |
| 4 | 0.0000 | 0.0000 | 0.0000 | 0.0000 | 0.0000 | 0.1850 |
| 5 | 0.0000 | 0.0000 | 0.0000 | 0.0000 | 0.0000 | 0.1850 |
| 6 | 0.0000 | 0.0000 | 0.0000 | 0.0000 | 0.0000 | 0.1850 |
| 7 | 0.0000 | 0.0000 | 0.0000 | 0.0000 | 0.0000 | 0.1850 |
| 8 | 0.0000 | 0.0000 | 0.0000 | 0.0000 | 0.0000 | 0.1850 |
| 9 | 0.0000 | 0.0000 | 0.0000 | 0.0000 | 0.0000 | 0.1850 |
| 10 | 0.0000 | 0.0000 | 0.0000 | 0.0000 | 0.0000 | 0.1850 |
| 11 | 0.0000 | 0.0000 | 0.0000 | 0.0000 | 0.0000 | 0.1850 |
| 12 | 0.0000 | 0.0000 | 0.0000 | 0.0000 | 0.0000 | 0.1850 |
| 13 | 0.0000 | 0.0000 | 0.0000 | 0.0000 | 0.0000 | 0.1850 |
| 14 | 0.0000 | 0.0000 | 0.0000 | 0.0000 | 0.0000 | 0.1850 |
| 15 | 0.0000 | 0.0000 | 0.0000 | 0.0000 | 0.0001 | 0.1850 |
| 16 | 0.0000 | 0.0000 | 0.0000 | 0.0000 | 0.0002 | 0.1850 |
| 17 | 0.0000 | 0.0000 | 0.0000 | 0.0000 | 0.0003 | 0.1850 |
| 18 | 0.0000 | 0.0000 | 0.0000 | 0.0000 | 0.0004 | 0.1850 |
| 19 | 0.0000 | 0.0000 | 0.0000 | 0.0000 | 0.0006 | 0.1850 |
| 20 | 0.9000 | 0.0000 | 0.0000 | 0.0001 | 0.0009 | 0.1850 |
| 21 | 0.9000 | 0.0000 | 0.0000 | 0.0002 | 0.0012 | 0.1850 |
| 22 | 0.9000 | 0.0000 | 0.0000 | 0.0003 | 0.0015 | 0.1850 |
| 23 | 0.9000 | 0.0000 | 0.0000 | 0.0004 | 0.0018 | 0.1850 |
| 24 | 0.9000 | 0.0000 | 0.0000 | 0.0006 | 0.0021 | 0.1850 |
| 25 | 10.1000 | 0.0001 | 0.0001 | 0.0009 | 0.0027 | 0.1850 |
| 26 | 10.1000 | 0.0001 | 0.0002 | 0.0012 | 0.0034 | 0.1850 |
| 27 | 10.1000 | 0.0001 | 0.0003 | 0.0015 | 0.0040 | 0.1850 |
| 28 | 10.1000 | 0.0001 | 0.0004 | 0.0018 | 0.0047 | 0.1850 |
| 29 | 10.1000 | 0.0001 | 0.0006 | 0.0021 | 0.0053 | 0.1850 |
| 30 | 31.1000 | 0.0003 | 0.0009 | 0.0027 | 0.0067 | 0.1850 |
| 31 | 31.1000 | 0.0003 | 0.0012 | 0.0034 | 0.0081 | 0.1850 |
| 32 | 31.1000 | 0.0003 | 0.0015 | 0.0040 | 0.0095 | 0.1850 |
| 33 | 31.1000 | 0.0003 | 0.0018 | 0.0047 | 0.0108 | 0.1850 |
| 34 | 31.1000 | 0.0003 | 0.0021 | 0.0053 | 0.0122 | 0.1850 |
| 35 | 64.4000 | 0.0006 | 0.0027 | 0.0067 | 0.0144 | 0.1850 |
| 36 | 64.4000 | 0.0006 | 0.0034 | 0.0081 | 0.0165 | 0.1850 |
| 37 | 64.4000 | 0.0006 | 0.0040 | 0.0095 | 0.0187 | 0.1850 |
| 38 | 64.4000 | 0.0006 | 0.0047 | 0.0108 | 0.0208 | 0.1850 |
| 39 | 64.4000 | 0.0006 | 0.0053 | 0.0122 | 0.0230 | 0.1850 |
| 40 | 137.6000 | 0.0014 | 0.0067 | 0.0144 | 0.0255 | 0.1850 |
| 41 | 137.6000 | 0.0014 | 0.0081 | 0.0165 | 0.0280 | 0.1850 |
| 42 | 137.6000 | 0.0014 | 0.0095 | 0.0187 | 0.0305 | 0.1850 |
| 43 | 137.6000 | 0.0014 | 0.0108 | 0.0208 | 0.0331 | 0.1850 |
| 44 | 137.6000 | 0.0014 | 0.0122 | 0.0230 | 0.0356 | 0.1850 |
| 45 | 215.8000 | 0.0022 | 0.0144 | 0.0255 | 0.0384 | 0.1850 |
| 46 | 215.8000 | 0.0022 | 0.0165 | 0.0280 | 0.0412 | 0.1850 |
| 47 | 215.8000 | 0.0022 | 0.0187 | 0.0305 | 0.0441 | 0.1850 |
| 48 | 215.8000 | 0.0022 | 0.0208 | 0.0331 | 0.0469 | 0.1850 |
| 49 | 215.8000 | 0.0022 | 0.0230 | 0.0356 | 0.0498 | 0.1850 |
| 50 | 251.4000 | 0.0025 | 0.0255 | 0.0384 | 0.0533 | 0.1850 |
| 51 | 251.4000 | 0.0025 | 0.0280 | 0.0412 | 0.0568 | 0.1850 |
| 52 | 251.4000 | 0.0025 | 0.0305 | 0.0441 | 0.0603 | 0.1850 |
| 53 | 251.4000 | 0.0025 | 0.0331 | 0.0469 | 0.0638 | 0.1850 |
| 54 | 251.4000 | 0.0025 | 0.0356 | 0.0498 | 0.0673 | 0.1850 |
| 55 | 283.8000 | 0.0028 | 0.0384 | 0.0533 | 0.0717 | 0.1850 |
| 56 | 283.8000 | 0.0028 | 0.0412 | 0.0568 | 0.0761 | 0.1850 |
| 57 | 283.8000 | 0.0028 | 0.0441 | 0.0603 | 0.0806 | 0.1850 |
| 58 | 283.8000 | 0.0028 | 0.0469 | 0.0638 | 0.0850 | 0.1850 |
| 59 | 283.8000 | 0.0028 | 0.0498 | 0.0673 | 0.0894 | 0.1850 |
| 60 | 351.4000 | 0.0035 | 0.0533 | 0.0717 | 0.0945 | 0.1850 |
| 61 | 351.4000 | 0.0035 | 0.0568 | 0.0761 | 0.0997 | 0.1850 |
| 62 | 351.4000 | 0.0035 | 0.0603 | 0.0806 | 0.1048 | 0.1850 |
| 63 | 351.4000 | 0.0035 | 0.0638 | 0.0850 | 0.1099 | 0.1850 |
| 64 | 351.4000 | 0.0035 | 0.0673 | 0.0894 | 0.1151 | 0.1850 |
| 65 | 441.0000 | 0.0044 | 0.0717 | 0.0945 | 0.1202 | 0.1850 |
| 66 | 441.0000 | 0.0044 | 0.0761 | 0.0997 | 0.1253 | 0.1850 |
| 67 | 441.0000 | 0.0044 | 0.0806 | 0.1048 | 0.1304 | 0.1850 |
| 68 | 441.0000 | 0.0044 | 0.0850 | 0.1099 | 0.1355 | 0.1850 |
| 69 | 441.0000 | 0.0044 | 0.0894 | 0.1151 | 0.1406 | 0.1850 |
| 70 | 513.8000 | 0.0051 | 0.0945 | 0.1202 | 0.1453 | 0.1850 |
| 71 | 513.8000 | 0.0051 | 0.0997 | 0.1253 | 0.1499 | 0.1850 |
| 72 | 513.8000 | 0.0051 | 0.1048 | 0.1304 | 0.1545 | 0.1850 |
| 73 | 513.8000 | 0.0051 | 0.1099 | 0.1355 | 0.1591 | 0.1850 |
| 74 | 513.8000 | 0.0051 | 0.1151 | 0.1406 | 0.1637 | 0.1850 |
| 75 | 511.4000 | 0.0051 | 0.1202 | 0.1453 | 0.1673 | 0.1850 |
| 76 | 511.4000 | 0.0051 | 0.1253 | 0.1499 | 0.1708 | 0.1850 |
| 77 | 511.4000 | 0.0051 | 0.1304 | 0.1545 | 0.1743 | 0.1850 |
| 78 | 511.4000 | 0.0051 | 0.1355 | 0.1591 | 0.1779 | 0.1850 |
| 79 | 511.4000 | 0.0051 | 0.1406 | 0.1637 | 0.1814 | 0.1850 |
| 80 | 462.0000 | 0.0046 | 0.1453 | 0.1673 | 0.1850 | 0.1850 |
| 81 | 462.0000 | 0.0046 | 0.1499 | 0.1708 | | 0.1850 |
| 82 | 462.0000 | 0.0046 | 0.1545 | 0.1743 | | 0.1850 |
| 83 | 462.0000 | 0.0046 | 0.1591 | 0.1779 | | 0.1850 |
| 84 | 462.0000 | 0.0046 | 0.1637 | 0.1814 | | 0.1850 |
| 85 | 353.7000 | 0.0035 | 0.1673 | 0.1850 | | 0.1850 |
| 86 | 353.7000 | 0.0035 | | | | 0.1850 |
| 87 | 353.7000 | 0.0035 | | | | 0.1850 |
| 88 | 353.7000 | 0.0035 | | | | 0.1850 |
| 89 | 353.7000 | 0.0035 | | | | 0.1850 |
| 90 | 353.7000 | 0.0035 | | | | 0.1850 |

TABLE 6
Look up table for African-American United States specific
population-based breast cancer incidence data.

| Age | incidence | incid | incid_b | incid_b_5 | incid_b_10 | incid_life |
|---|---|---|---|---|---|---|
| 0.0000 | 0.0000 | 0.0000 | 0.0000 | 0.0000 | 0.0000 | 0.1707 |
| 1.0000 | 0.0000 | 0.0000 | 0.0000 | 0.0000 | 0.0000 | 0.1707 |
| 2.0000 | 0.0000 | 0.0000 | 0.0000 | 0.0000 | 0.0000 | 0.1707 |
| 3.0000 | 0.0000 | 0.0000 | 0.0000 | 0.0000 | 0.0000 | 0.1707 |
| 4.0000 | 0.0000 | 0.0000 | 0.0000 | 0.0000 | 0.0000 | 0.1707 |
| 5.0000 | 0.0000 | 0.0000 | 0.0000 | 0.0000 | 0.0000 | 0.1707 |
| 6.0000 | 0.0000 | 0.0000 | 0.0000 | 0.0000 | 0.0000 | 0.1707 |
| 7.0000 | 0.0000 | 0.0000 | 0.0000 | 0.0000 | 0.0000 | 0.1707 |
| 8.0000 | 0.0000 | 0.0000 | 0.0000 | 0.0000 | 0.0000 | 0.1707 |
| 9.0000 | 0.0000 | 0.0000 | 0.0000 | 0.0000 | 0.0000 | 0.1707 |
| 10.0000 | 0.0000 | 0.0000 | 0.0000 | 0.0000 | 0.0000 | 0.1707 |
| 11.0000 | 0.0000 | 0.0000 | 0.0000 | 0.0000 | 0.0001 | 0.1707 |
| 12.0000 | 0.0000 | 0.0000 | 0.0000 | 0.0000 | 0.0001 | 0.1707 |
| 13.0000 | 0.0000 | 0.0000 | 0.0000 | 0.0000 | 0.0001 | 0.1707 |
| 14.0000 | 0.0000 | 0.0000 | 0.0000 | 0.0000 | 0.0001 | 0.1707 |
| 15.0000 | 0.3000 | 0.0000 | 0.0000 | 0.0000 | 0.0003 | 0.1707 |
| 16.0000 | 0.3000 | 0.0000 | 0.0000 | 0.0001 | 0.0004 | 0.1707 |
| 17.0000 | 0.3000 | 0.0000 | 0.0000 | 0.0001 | 0.0006 | 0.1707 |
| 18.0000 | 0.3000 | 0.0000 | 0.0000 | 0.0001 | 0.0007 | 0.1707 |
| 19.0000 | 0.3000 | 0.0000 | 0.0000 | 0.0001 | 0.0008 | 0.1707 |
| 20.0000 | 2.2000 | 0.0000 | 0.0000 | 0.0003 | 0.0012 | 0.1707 |
| 21.0000 | 2.2000 | 0.0000 | 0.0001 | 0.0004 | 0.0016 | 0.1707 |
| 22.0000 | 2.2000 | 0.0000 | 0.0001 | 0.0006 | 0.0019 | 0.1707 |
| 23.0000 | 2.2000 | 0.0000 | 0.0001 | 0.0007 | 0.0023 | 0.1707 |
| 24.0000 | 2.2000 | 0.0000 | 0.0001 | 0.0008 | 0.0027 | 0.1707 |
| 25.0000 | 14.3000 | 0.0001 | 0.0003 | 0.0012 | 0.0034 | 0.1707 |
| 26.0000 | 14.3000 | 0.0001 | 0.0004 | 0.0016 | 0.0041 | 0.1707 |
| 27.0000 | 14.3000 | 0.0001 | 0.0006 | 0.0019 | 0.0047 | 0.1707 |
| 28.0000 | 14.3000 | 0.0001 | 0.0007 | 0.0023 | 0.0054 | 0.1707 |
| 29.0000 | 14.3000 | 0.0001 | 0.0008 | 0.0027 | 0.0061 | 0.1707 |
| 30.0000 | 36.4000 | 0.0004 | 0.0012 | 0.0034 | 0.0075 | 0.1707 |
| 31.0000 | 36.4000 | 0.0004 | 0.0016 | 0.0041 | 0.0088 | 0.1707 |
| 32.0000 | 36.4000 | 0.0004 | 0.0019 | 0.0047 | 0.0101 | 0.1707 |
| 33.0000 | 36.4000 | 0.0004 | 0.0023 | 0.0054 | 0.0114 | 0.1707 |
| 34.0000 | 36.4000 | 0.0004 | 0.0027 | 0.0061 | 0.0127 | 0.1707 |
| 35.0000 | 69.6000 | 0.0007 | 0.0034 | 0.0075 | 0.0146 | 0.1707 |
| 36.0000 | 69.6000 | 0.0007 | 0.0041 | 0.0088 | 0.0166 | 0.1707 |
| 37.0000 | 69.6000 | 0.0007 | 0.0047 | 0.0101 | 0.0185 | 0.1707 |
| 38.0000 | 69.6000 | 0.0007 | 0.0054 | 0.0114 | 0.0205 | 0.1707 |
| 39.0000 | 69.6000 | 0.0007 | 0.0061 | 0.0127 | 0.0224 | 0.1707 |
| 40.0000 | 131.3000 | 0.0013 | 0.0075 | 0.0146 | 0.0245 | 0.1707 |
| 41.0000 | 131.3000 | 0.0013 | 0.0088 | 0.0166 | 0.0266 | 0.1707 |
| 42.0000 | 131.3000 | 0.0013 | 0.0101 | 0.0185 | 0.0287 | 0.1707 |
| 43.0000 | 131.3000 | 0.0013 | 0.0114 | 0.0205 | 0.0308 | 0.1707 |
| 44.0000 | 131.3000 | 0.0013 | 0.0127 | 0.0224 | 0.0329 | 0.1707 |
| 45.0000 | 193.7000 | 0.0019 | 0.0146 | 0.0245 | 0.0356 | 0.1707 |
| 46.0000 | 193.7000 | 0.0019 | 0.0166 | 0.0266 | 0.0382 | 0.1707 |
| 47.0000 | 193.7000 | 0.0019 | 0.0185 | 0.0287 | 0.0408 | 0.1707 |
| 48.0000 | 193.7000 | 0.0019 | 0.0205 | 0.0308 | 0.0434 | 0.1707 |
| 49.0000 | 193.7000 | 0.0019 | 0.0224 | 0.0329 | 0.0461 | 0.1707 |
| 50.0000 | 210.7000 | 0.0021 | 0.0245 | 0.0356 | 0.0495 | 0.1707 |
| 51.0000 | 210.7000 | 0.0021 | 0.0266 | 0.0382 | 0.0530 | 0.1707 |
| 52.0000 | 210.7000 | 0.0021 | 0.0287 | 0.0408 | 0.0564 | 0.1707 |
| 53.0000 | 210.7000 | 0.0021 | 0.0308 | 0.0434 | 0.0599 | 0.1707 |
| 54.0000 | 210.7000 | 0.0021 | 0.0329 | 0.0461 | 0.0634 | 0.1707 |
| 55.0000 | 263.1000 | 0.0026 | 0.0356 | 0.0495 | 0.0675 | 0.1707 |
| 56.0000 | 263.1000 | 0.0026 | 0.0382 | 0.0530 | 0.0716 | 0.1707 |
| 57.0000 | 263.1000 | 0.0026 | 0.0408 | 0.0564 | 0.0758 | 0.1707 |
| 58.0000 | 263.1000 | 0.0026 | 0.0434 | 0.0599 | 0.0799 | 0.1707 |
| 59.0000 | 263.1000 | 0.0026 | 0.0461 | 0.0634 | 0.0841 | 0.1707 |
| 60.0000 | 345.5000 | 0.0035 | 0.0495 | 0.0675 | 0.0886 | 0.1707 |
| 61.0000 | 345.5000 | 0.0035 | 0.0530 | 0.0716 | 0.0931 | 0.1707 |
| 62.0000 | 345.5000 | 0.0035 | 0.0564 | 0.0758 | 0.0976 | 0.1707 |
| 63.0000 | 345.5000 | 0.0035 | 0.0599 | 0.0799 | 0.1021 | 0.1707 |
| 64.0000 | 345.5000 | 0.0035 | 0.0634 | 0.0841 | 0.1066 | 0.1707 |
| 65.0000 | 414.0000 | 0.0041 | 0.0675 | 0.0886 | 0.1109 | 0.1707 |
| 66.0000 | 414.0000 | 0.0041 | 0.0716 | 0.0931 | 0.1152 | 0.1707 |
| 67.0000 | 414.0000 | 0.0041 | 0.0758 | 0.0976 | 0.1196 | 0.1707 |
| 68.0000 | 414.0000 | 0.0041 | 0.0799 | 0.1021 | 0.1239 | 0.1707 |
| 69.0000 | 414.0000 | 0.0041 | 0.0841 | 0.1066 | 0.1282 | 0.1707 |
| 70.0000 | 450.6000 | 0.0045 | 0.0886 | 0.1109 | 0.1323 | 0.1707 |
| 71.0000 | 450.6000 | 0.0045 | 0.0931 | 0.1152 | 0.1364 | 0.1707 |
| 72.0000 | 450.6000 | 0.0045 | 0.0976 | 0.1196 | 0.1405 | 0.1707 |
| 73.0000 | 450.6000 | 0.0045 | 0.1021 | 0.1239 | 0.1446 | 0.1707 |
| 74.0000 | 450.6000 | 0.0045 | 0.1066 | 0.1282 | 0.1486 | 0.1707 |
| 75.0000 | 433.1000 | 0.0043 | 0.1109 | 0.1323 | 0.1523 | 0.1707 |
| 76.0000 | 433.1000 | 0.0043 | 0.1152 | 0.1364 | 0.1560 | 0.1707 |
| 77.0000 | 433.1000 | 0.0043 | 0.1196 | 0.1405 | 0.1597 | 0.1707 |
| 78.0000 | 433.1000 | 0.0043 | 0.1239 | 0.1446 | 0.1634 | 0.1707 |
| 79.0000 | 433.1000 | 0.0043 | 0.1282 | 0.1486 | 0.1670 | 0.1707 |
| 80.0000 | 407.8000 | 0.0041 | 0.1323 | 0.1523 | 0.1707 | 0.1707 |
| 81.0000 | 407.8000 | 0.0041 | 0.1364 | 0.1560 | | 0.1707 |
| 82.0000 | 407.8000 | 0.0041 | 0.1405 | 0.1597 | | 0.1707 |
| 83.0000 | 407.8000 | 0.0041 | 0.1446 | 0.1634 | | 0.1707 |
| 84.0000 | 407.8000 | 0.0041 | 0.1486 | 0.1670 | | 0.1707 |
| 85.0000 | 368.1000 | 0.0037 | 0.1523 | 0.1707 | | 0.1707 |
| 86.0000 | 368.1000 | 0.0037 | | | | 0.1707 |
| 87.0000 | 368.1000 | 0.0037 | | | | 0.1707 |
| 88.0000 | 368.1000 | 0.0037 | | | | 0.1707 |
| 89.0000 | 368.1000 | 0.0037 | | | | 0.1707 |
| 90.0000 | 368.1000 | 0.0037 | | | | 0.1707 |

TABLE 7
Look up table for Hispanic United States specific
population-based breast cancer incidence data.

| Age | incidence | incid | incid_b | incid_b_5 | incid_b_10 | incid_life |
|---|---|---|---|---|---|---|
| 0.0000 | 0.0000 | 0.0000 | 0.0000 | 0.0000 | 0.0000 | 0.1280 |
| 1.0000 | 0.0000 | 0.0000 | 0.0000 | 0.0000 | 0.0000 | 0.1280 |
| 2.0000 | 0.0000 | 0.0000 | 0.0000 | 0.0000 | 0.0000 | 0.1280 |
| 3.0000 | 0.0000 | 0.0000 | 0.0000 | 0.0000 | 0.0000 | 0.1280 |
| 4.0000 | 0.0000 | 0.0000 | 0.0000 | 0.0000 | 0.0000 | 0.1280 |
| 5.0000 | 0.0000 | 0.0000 | 0.0000 | 0.0000 | 0.0000 | 0.1280 |
| 6.0000 | 0.0000 | 0.0000 | 0.0000 | 0.0000 | 0.0000 | 0.1280 |
| 7.0000 | 0.0000 | 0.0000 | 0.0000 | 0.0000 | 0.0000 | 0.1280 |
| 8.0000 | 0.0000 | 0.0000 | 0.0000 | 0.0000 | 0.0000 | 0.1280 |
| 9.0000 | 0.0000 | 0.0000 | 0.0000 | 0.0000 | 0.0000 | 0.1280 |
| 10.0000 | 0.0000 | 0.0000 | 0.0000 | 0.0000 | 0.0000 | 0.1280 |
| 11.0000 | 0.0000 | 0.0000 | 0.0000 | 0.0000 | 0.0001 | 0.1280 |
| 12.0000 | 0.0000 | 0.0000 | 0.0000 | 0.0000 | 0.0001 | 0.1280 |
| 13.0000 | 0.0000 | 0.0000 | 0.0000 | 0.0000 | 0.0001 | 0.1280 |
| 14.0000 | 0.0000 | 0.0000 | 0.0000 | 0.0000 | 0.0001 | 0.1280 |
| 15.0000 | 0.3000 | 0.0000 | 0.0000 | 0.0000 | 0.0002 | 0.1280 |
| 16.0000 | 0.3000 | 0.0000 | 0.0000 | 0.0001 | 0.0003 | 0.1280 |
| 17.0000 | 0.3000 | 0.0000 | 0.0000 | 0.0001 | 0.0004 | 0.1280 |
| 18.0000 | 0.3000 | 0.0000 | 0.0000 | 0.0001 | 0.0005 | 0.1280 |
| 19.0000 | 0.3000 | 0.0000 | 0.0000 | 0.0001 | 0.0005 | 0.1280 |
| 20.0000 | 1.9000 | 0.0000 | 0.0000 | 0.0002 | 0.0008 | 0.1280 |
| 21.0000 | 1.9000 | 0.0000 | 0.0001 | 0.0003 | 0.0010 | 0.1280 |
| 22.0000 | 1.9000 | 0.0000 | 0.0001 | 0.0004 | 0.0012 | 0.1280 |
| 23.0000 | 1.9000 | 0.0000 | 0.0001 | 0.0005 | 0.0015 | 0.1280 |
| 24.0000 | 1.9000 | 0.0000 | 0.0001 | 0.0005 | 0.0017 | 0.1280 |
| 25.0000 | 8.5000 | 0.0001 | 0.0002 | 0.0008 | 0.0022 | 0.1280 |
| 26.0000 | 8.5000 | 0.0001 | 0.0003 | 0.0010 | 0.0027 | 0.1280 |
| 27.0000 | 8.5000 | 0.0001 | 0.0004 | 0.0012 | 0.0031 | 0.1280 |
| 28.0000 | 8.5000 | 0.0001 | 0.0005 | 0.0015 | 0.0036 | 0.1280 |
| 29.0000 | 8.5000 | 0.0001 | 0.0005 | 0.0017 | 0.0041 | 0.1280 |
| 30.0000 | 23.4000 | 0.0002 | 0.0008 | 0.0022 | 0.0051 | 0.1280 |
| 31.0000 | 23.4000 | 0.0002 | 0.0010 | 0.0027 | 0.0062 | 0.1280 |
| 32.0000 | 23.4000 | 0.0002 | 0.0012 | 0.0031 | 0.0072 | 0.1280 |
| 33.0000 | 23.4000 | 0.0002 | 0.0015 | 0.0036 | 0.0082 | 0.1280 |
| 34.0000 | 23.4000 | 0.0002 | 0.0017 | 0.0041 | 0.0093 | 0.1280 |
| 35.0000 | 47.6000 | 0.0005 | 0.0022 | 0.0051 | 0.0107 | 0.1280 |
| 36.0000 | 47.6000 | 0.0005 | 0.0027 | 0.0062 | 0.0121 | 0.1280 |
| 37.0000 | 47.6000 | 0.0005 | 0.0031 | 0.0072 | 0.0136 | 0.1280 |
| 38.0000 | 47.6000 | 0.0005 | 0.0036 | 0.0082 | 0.0150 | 0.1280 |
| 39.0000 | 47.6000 | 0.0005 | 0.0041 | 0.0093 | 0.0164 | 0.1280 |
| 40.0000 | 103.4000 | 0.0010 | 0.0051 | 0.0107 | 0.0182 | 0.1280 |
| 41.0000 | 103.4000 | 0.0010 | 0.0062 | 0.0121 | 0.0200 | 0.1280 |
| 42.0000 | 103.4000 | 0.0010 | 0.0072 | 0.0136 | 0.0218 | 0.1280 |
| 43.0000 | 103.4000 | 0.0010 | 0.0082 | 0.0150 | 0.0236 | 0.1280 |
| 44.0000 | 103.4000 | 0.0010 | 0.0093 | 0.0164 | 0.0254 | 0.1280 |
| 45.0000 | 143.8000 | 0.0014 | 0.0107 | 0.0182 | 0.0276 | 0.1280 |
| 46.0000 | 143.8000 | 0.0014 | 0.0121 | 0.0200 | 0.0297 | 0.1280 |
| 47.0000 | 143.8000 | 0.0014 | 0.0136 | 0.0218 | 0.0319 | 0.1280 |
| 48.0000 | 143.8000 | 0.0014 | 0.0150 | 0.0236 | 0.0340 | 0.1280 |
| 49.0000 | 143.8000 | 0.0014 | 0.0164 | 0.0254 | 0.0362 | 0.1280 |
| 50.0000 | 179.1000 | 0.0018 | 0.0182 | 0.0276 | 0.0388 | 0.1280 |
| 51.0000 | 179.1000 | 0.0018 | 0.0200 | 0.0297 | 0.0415 | 0.1280 |
| 52.0000 | 179.1000 | 0.0018 | 0.0218 | 0.0319 | 0.0442 | 0.1280 |
| 53.0000 | 179.1000 | 0.0018 | 0.0236 | 0.0340 | 0.0469 | 0.1280 |
| 54.0000 | 179.1000 | 0.0018 | 0.0254 | 0.0362 | 0.0496 | 0.1280 |
| 55.0000 | 215.0000 | 0.0022 | 0.0276 | 0.0388 | 0.0529 | 0.1280 |
| 56.0000 | 215.0000 | 0.0022 | 0.0297 | 0.0415 | 0.0563 | 0.1280 |
| 57.0000 | 215.0000 | 0.0022 | 0.0319 | 0.0442 | 0.0596 | 0.1280 |
| 58.0000 | 215.0000 | 0.0022 | 0.0340 | 0.0469 | 0.0630 | 0.1280 |
| 59.0000 | 215.0000 | 0.0022 | 0.0362 | 0.0496 | 0.0663 | 0.1280 |
| 60.0000 | 268.0000 | 0.0027 | 0.0388 | 0.0529 | 0.0700 | 0.1280 |
| 61.0000 | 268.0000 | 0.0027 | 0.0415 | 0.0563 | 0.0738 | 0.1280 |
| 62.0000 | 268.0000 | 0.0027 | 0.0442 | 0.0596 | 0.0775 | 0.1280 |
| 63.0000 | 268.0000 | 0.0027 | 0.0469 | 0.0630 | 0.0813 | 0.1280 |
| 64.0000 | 268.0000 | 0.0027 | 0.0496 | 0.0663 | 0.0850 | 0.1280 |
| 65.0000 | 335.0000 | 0.0034 | 0.0529 | 0.0700 | 0.0879 | 0.1280 |
| 66.0000 | 335.0000 | 0.0034 | 0.0563 | 0.0738 | 0.0908 | 0.1280 |
| 67.0000 | 335.0000 | 0.0034 | 0.0596 | 0.0775 | 0.0936 | 0.1280 |
| 68.0000 | 335.0000 | 0.0034 | 0.0630 | 0.0813 | 0.0965 | 0.1280 |
| 69.0000 | 335.0000 | 0.0034 | 0.0663 | 0.0850 | 0.0994 | 0.1280 |
| 70.0000 | 374.1000 | 0.0037 | 0.0700 | 0.0879 | 0.1023 | 0.1280 |
| 71.0000 | 374.1000 | 0.0037 | 0.0738 | 0.0908 | 0.1052 | 0.1280 |
| 72.0000 | 374.1000 | 0.0037 | 0.0775 | 0.0936 | 0.1081 | 0.1280 |
| 73.0000 | 374.1000 | 0.0037 | 0.0813 | 0.0965 | 0.1110 | 0.1280 |
| 74.0000 | 374.1000 | 0.0037 | 0.0850 | 0.0994 | 0.1139 | 0.1280 |
| 75.0000 | 288.1000 | 0.0029 | 0.0879 | 0.1023 | 0.1162 | 0.1280 |
| 76.0000 | 288.1000 | 0.0029 | 0.0908 | 0.1052 | 0.1186 | 0.1280 |
| 77.0000 | 288.1000 | 0.0029 | 0.0936 | 0.1081 | 0.1209 | 0.1280 |
| 78.0000 | 288.1000 | 0.0029 | 0.0965 | 0.1110 | 0.1233 | 0.1280 |
| 79.0000 | 288.1000 | 0.0029 | 0.0994 | 0.1139 | 0.1256 | 0.1280 |
| 80.0000 | 289.0000 | 0.0029 | 0.1023 | 0.1162 | 0.1280 | 0.1280 |
| 81.0000 | 289.0000 | 0.0029 | 0.1052 | 0.1186 | | 0.1280 |
| 82.0000 | 289.0000 | 0.0029 | 0.1081 | 0.1209 | | 0.1280 |
| 83.0000 | 289.0000 | 0.0029 | 0.1110 | 0.1233 | | 0.1280 |
| 84.0000 | 289.0000 | 0.0029 | 0.1139 | 0.1256 | | 0.1280 |
| 85.0000 | 235.2000 | 0.0024 | 0.1162 | 0.1280 | | 0.1280 |
| 86.0000 | 235.2000 | 0.0024 | | | | 0.1280 |
| 87.0000 | 235.2000 | 0.0024 | | | | 0.1280 |
| 88.0000 | 235.2000 | 0.0024 | | | | 0.1280 |
| 89.0000 | 235.2000 | 0.0024 | | | | 0.1280 |
| 90.0000 | 235.2000 | 0.0024 | | | | 0.1280 |

### Genetic Risk Assessment

Various exemplary polymorphisms associated with breast cancer are discussed in the present disclosure. These polymorphisms vary in terms of penetrance and many would be understood by those of skill in the art to be low penetrance polymorphisms.

The term “penetrance” is used in the context of the present disclosure to refer to the frequency at which a particular polymorphism manifests itself within female subjects with breast cancer. “High penetrance” polymorphisms will almost always be apparent in a female subject with breast cancer while “low penetrance” polymorphisms will only sometimes be apparent. In an embodiment polymorphisms assessed as part of a genetic risk assessment according to the present disclosure are low penetrance polymorphisms.

In an embodiment, the genetic risk assessment is performed by analysing the genotype of the subject at 72 or more loci for polymorphisms associated with breast cancer. In an embodiment, the genetic risk assessment is performed by analysing the genotype of the subject at 150 or more loci for polymorphisms associated with breast cancer. In an embodiment, the genetic risk assessment is performed by analysing the genotype of the subject at 200 or more loci for polymorphisms associated with breast cancer. In an embodiment, the genetic risk assessment is performed by analysing the genotype of the subject at 313 or more loci for polymorphisms associated with breast cancer. In an embodiment, the genetic risk assessment is performed by analysing the genotype of the subject at 1,500 or more loci for polymorphisms associated with breast cancer. In an embodiment, the genetic risk assessment is performed by analysing the genotype of the subject at 3,820 or more loci for polymorphisms associated with breast cancer.

In an embodiment, when performing the methods of the present disclosure to assess risk of breast cancer, at least 67 of the polymorphisms are selected from Table 79 or a polymorphism in linkage disequilibrium with one or more thereof and the remaining polymorphisms are selected from Table 8, or a polymorphism in linkage disequilibrium with one or more thereof. In another embodiment, when performing the methods of the present disclosure at least 68, at least 69, at least 70 of the polymorphisms are selected from Table 9 or a polymorphism in linkage disequilibrium with one or more thereof and the remaining polymorphisms are selected from Table 8, or a polymorphism in linkage disequilibrium with one or more thereof. In one embodiment, at least 72, at least 73, at least 74, at least 75, at least 76, at least 77, at least 78, at least 79, at least 80, at least 81, at least 82, at least 83, at least 84, at least 85, at least 86, at least 87, at least 88 of the polymorphisms shown in Table 8, or a polymorphism in linkage disequilibrium with one or more thereof are assessed. In further embodiments, at least 67, at least 68, at least 69, at least 70 of the polymorphisms shown in Table 8, or a polymorphism in linkage disequilibrium with one or more thereof are assessed. In further embodiments, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88 polymorphisms are assessed, wherein at least 67, at least 68, at least 69, at least 70 polymorphisms shown in Table 9, or a polymorphism in linkage disequilibrium with one or more thereof are assessed, with any remaining polymorphisms being selected from Table 8, or a polymorphism in linkage disequilibrium with one or more thereof.

In some embodiments, when performing the methods of the present disclosure to assess risk of breast cancer, one or more polymorphisms are selected from Table 14 or a polymorphism in linkage disequilibrium with one or more thereof. In an embodiment, at least 50 of the polymorphisms are selected from Table 14, or a polymorphism in linkage disequilibrium with one or more thereof. In an embodiment, at least 60, at least 70, at least 80, at least 90, at least 100, at least 110, at least 120, at least 130, at least 140, at least 150, at least 160, at least 170, at least 180, at least 190, at least 200 of the polymorphisms are selected from Table 14, or a polymorphism in linkage disequilibrium with one or more thereof. In an embodiment, at least 100 of the polymorphisms are selected from Table 14, or a polymorphism in linkage disequilibrium with one or more thereof. In an embodiment, at least 150 of the polymorphisms are selected from Table 14, or a polymorphism in linkage disequilibrium with one or more thereof. In an embodiment, at least 200 of the polymorphisms are selected from Table 14, or a polymorphism in linkage disequilibrium with one or more thereof.

In an embodiment, when determining breast cancer risk, the methods of the present disclosure comprise detecting at least 50, at least 100, or at least 150 polymorphisms shown in Table 14, or a polymorphism in linkage disequilibrium with one or more thereof. In an embodiment, when determining breast cancer risk, the methods of the present disclosure comprise detecting all of the 203 polymorphisms shown in Table 14, or a polymorphism in linkage disequilibrium with one or more thereof.

In an embodiment, when determining breast cancer risk, the methods of the present disclosure comprise detecting at least 50, 80, 100, 150 of the polymorphisms shown in Table 14, or a polymorphism in linkage disequilibrium with one or more thereof.

In another embodiment, when determining breast cancer risk, the methods of the present disclosure encompass assessing all of the polymorphisms shown in Table 8 or Table 14 or a polymorphism in linkage disequilibrium with one or more thereof.

Table 8, Table 9, and Table 14 recite overlapping polymorphisms. It will be appreciated that when selecting polymorphisms for assessment the same polymorphism will not be selected twice. For convenience, the polymorphisms in Table 8 have been separated into Tables 9 and 10. Table 9 lists polymorphisms common across Caucasians, African American and Hispanic populations. Table 10 lists polymorphisms that are not common across Caucasian, African American and Hispanic populations.

In a further embodiment, between 72 and 88, between 73 and 87, between 74 and 86, between 75 and 85, between 76 and 84, between 75 and 83, between 76 and 82, between 77 and 81, between 78 and 80 polymorphisms are assessed, wherein at least 60, at least 61, at least 62, at least 63, at least 64, at least 65, at least 66, at least 67, at least 68, at least 69, at least 70, of the polymorphisms shown in Table 9, or a polymorphism in linkage disequilibrium with one or more thereof are assessed, with any remaining polymorphisms being selected from Table 8, or a polymorphism in linkage disequilibrium with one or more thereof.

In an embodiment, when performing the methods of the present disclosure to assess risk of breast cancer, the 313 polymorphisms provided in Table 17 or a polymorphism in linkage disequilibrium with one or more thereof are assessed. In an embodiment, the subject is Caucasian.

In an embodiment, when performing the methods of the present disclosure to assess risk of breast cancer, the 3,820 polymorphisms provided in Table 18 or a polymorphism in linkage disequilibrium with one or more thereof are assessed. In an embodiment, the subject is Caucasian.

In an embodiment, a panel of polymorphisms described in WO 2010/139006, WO2016/049694 or Mavaddat et al. (2019) is used.

Polymorphisms in linkage disequilibrium with those specifically mentioned herein are easily identified by those of skill in the art, such as using the HAPMAP database as described in WO 2016/049694.

### Ethnic Genotype Variation

It is known to those of skill in the art that genotypic variation exists between different populations. This phenomenon is referred to as human genetic variation. Human genetic variation is often observed between populations from different ethnic backgrounds. Such variation is rarely consistent and is often directed by various combinations of environmental and lifestyle factors. As a result of genetic variation, it is often difficult to identify a population of genetic markers such as polymorphisms that remain informative across various populations such as populations from different ethnic backgrounds.

A selection of polymorphisms that are common to at least three ethnic backgrounds and remain informative for assessing the risk for developing breast cancer are disclosed herein.

In an embodiment, the methods of the present disclosure can be used for assessing the risk for developing breast cancer in human female subjects from various ethnic backgrounds. For example, the female subject can be classified as Caucasoid, Australoid, Mongoloid and Negroid based on physical anthropology.

In an embodiment, the human female subject can be Caucasian, African American, Hispanic, Asian, Indian, or Latina. In a preferred embodiment, the human female subject is Caucasian, African American or Hispanic. Accordingly, ethnicity can be taken into consideration as part of the clinical and/or genetic risk assessments.

In one embodiment, the human female subject is Caucasian and at least 72, at least 73, at least 74, at least 75, at least 76, at least 77, polymorphisms selected from Table 11, or a polymorphism in linkage disequilibrium with one or more thereof are assessed. Alternatively, all of the 77 polymorphisms selected from Table 11 or a polymorphism in linkage disequilibrium with one or more thereof are assessed.

In another embodiment, the human female subject is Negroid or African American and at least 70, at least 71, at least 72, at least 73, or at least 74 polymorphisms selected from Table 12, or a polymorphism in linkage disequilibrium with one or more thereof are assessed. Alternatively, at least 74 polymorphisms selected from Table 12 or a polymorphism in linkage disequilibrium with one or more thereof are assessed.

In another embodiment, the human female subject is Negroid or African American and at least 70, at least 71, at least 72, at least 73, or at least 74 polymorphisms shown in Table 15, or a polymorphism in linkage disequilibrium with one or more thereof are assessed. In one embodiment, the human female subject is Negroid or African American and the methods described herein comprise detecting all of the 74 polymorphisms shown in Table 15, or a polymorphism in linkage disequilibrium with one or more thereof.

In a further embodiment, the human female subject is Hispanic and at least 67, at least 68, at least 69, at least 70, or at least 71 polymorphisms selected from Table 13, or a polymorphism in linkage disequilibrium with one or more thereof are assessed. Alternatively, at least 71 polymorphisms selected from Table 13 or a polymorphism in linkage disequilibrium with one or more thereof are assessed.

In another embodiment, the human female subject can be Hispanic and at least 67, at least 68, at least 69, at least 70, or at least 71 polymorphisms shown in Table 16, or a polymorphism in linkage disequilibrium with one or more thereof are assessed. In one embodiment, the human female subject can be Hispanic and the methods described herein comprise detecting all of the 71 polymorphisms shown in Table 16, or a polymorphism in linkage disequilibrium with one or more thereof.

It is well known that over time there has been blending of different ethnic origins. However, in practice this does not influence the ability of a skilled person to practice the invention.

A female subject of predominantly European origin, either direct or indirect through ancestry, with white skin is considered Caucasian in the context of the present disclosure. A Caucasian may have, for example, at least 75% Caucasian ancestry (for example, but not limited to, the female subject having at least three Caucasian grandparents).

A female subject of predominantly central or southern African origin, either direct or indirect through ancestry, is considered Negroid in the context of the present disclosure. A Negroid may have, for example, at least 75% Negroid ancestry. An American female subject with predominantly Negroid ancestry and black skin is considered African American in the context of the present disclosure. An African American may have, for example, at least 75% Negroid ancestry. Similar principle applies to, for example, females of Negroid ancestry living in other countries (for example Great Britain, Canada and The Netherlands).

A female subject predominantly originating from Spain or a Spanish-speaking country, such as a country of Central or Southern America, either direct or indirect through ancestry, is considered Hispanic in the context of the present disclosure. An Hispanic may have, for example, at least 75% Hispanic ancestry.

The terms “ethnicity” and “race” can be used interchangeably in the context of the present disclosure. In an embodiment, the genetic risk assessment can readily be practiced based on what ethnicity the subject considers themselves to be. Thus, in an embodiment, the ethnicity of the human female subject is self-reported by the subject. As an example, female subjects can be asked to identify their ethnicity in response to this question: “To what ethnic group do you belong?”. In another example, the ethnicity of the female subject is derived from medical records after obtaining the appropriate consent from the subject or from the opinion or observations of a clinician.

TABLE 11
Examples of Caucasian polymorphisms (n = 77). Alleles represented as major/minor (e.g. for
rs616488 A is the common allele and G less common). Minor allele odds ratios below 1 mean the
minor allele is not the risk allele, whereas when above 1 the minor allele is the risk allele.

| | | | Minor | OR | | | | | | | |
|---|---|---|---|---|---|---|---|---|---|---|---|
| | | | allele | Minor | | | | | | | |
| Polymorphism | Chromosome | Alleles | frequency | Allele | μ | Adjusted Risk Score | | | | | |
| rs616488 | 1 | A/G | 0.33 | 0.9417 | 0.96 | AA | 1.04 | GA | 0.98 | GG | 0.92 |
| rs11552449 | 1 | C/T | 0.17 | 1.0810 | 1.03 | CC | 0.97 | TC | 1.05 | TT | 1.14 |
| rs11249433 | 1 | A/G | 0.40 | 1.0993 | 1.08 | AA | 0.93 | GA | 1.02 | GG | 1.12 |
| rs6678914 | 1 | G/A | 0.414 | 0.9890 | 0.99 | GG | 1.01 | AG | 1.00 | AA | 0.99 |
| rs4245739 | 1 | A/C | 0.258 | 1.0291 | 1.02 | AA | 0.99 | CA | 1.01 | CC | 1.04 |
| rs12710696 | 2 | G/A | 0.357 | 1.0387 | 1.03 | GG | 0.97 | AG | 1.01 | AA | 1.05 |
| rs4849887 | 2 | C/T | 0.098 | 0.9187 | 0.98 | CC | 1.02 | TC | 0.93 | TT | 0.86 |
| rs2016394 | 2 | G/A | 0.48 | 0.9504 | 0.95 | GG | 1.05 | AG | 1.00 | AA | 0.95 |
| rs1550623 | 2 | A/G | 0.16 | 0.9445 | 0.98 | AA | 1.02 | GA | 0.96 | GG | 0.91 |
| rs1045485 | 2 | G/C | 0.13 | 0.9644 | 0.99 | GG | 1.01 | CG | 0.97 | CC | 0.94 |
| rs13387042 | 2 | A/G | 0.49 | 0.8794 | 0.89 | AA | 1.13 | GA | 0.99 | GG | 0.87 |
| rs16857609 | 2 | C/T | 0.26 | 1.0721 | 1.04 | CC | 0.96 | TC | 1.03 | TT | 1.11 |
| rs6762644 | 3 | A/G | 0.4 | 1.0661 | 1.05 | AA | 0.95 | GA | 1.01 | GG | 1.08 |
| rs4973768 | 3 | C/T | 0.47 | 1.0938 | 1.09 | CC | 0.92 | TC | 1.00 | TT | 1.10 |
| rs12493607 | 3 | G/C | 0.35 | 1.0529 | 1.04 | GG | 0.96 | CG | 1.01 | CC | 1.07 |
| rs9790517 | 4 | C/T | 0.23 | 1.0481 | 1.02 | CC | 0.98 | TC | 1.03 | TT | 1.07 |
| rs6828523 | 4 | C/A | 0.13 | 0.9056 | 0.98 | CC | 1.03 | AC | 0.93 | AA | 0.84 |
| rs10069690 | 5 | C/T | 0.26 | 1.0242 | 1.01 | CC | 0.99 | TC | 1.01 | TT | 1.04 |
| rs7726159 | 5 | C/A | 0.338 | 1.0359 | 1.02 | CC | 0.98 | AC | 1.01 | AA | 1.05 |
| rs2736108 | 5 | C/T | 0.292 | 0.9379 | 0.96 | CC | 1.04 | TC | 0.97 | TT | 0.91 |
| rs10941679 | 5 | A/G | 0.25 | 1.1198 | 1.06 | AA | 0.94 | GA | 1.06 | GG | 1.18 |
| rs889312 | 5 | A/C | 0.28 | 1.1176 | 1.07 | AA | 0.94 | CA | 1.05 | CC | 1.17 |
| rs10472076 | 5 | T/C | 0.38 | 1.0419 | 1.03 | TT | 0.97 | CT | 1.01 | CC | 1.05 |
| rs1353747 | 5 | T/G | 0.095 | 0.9213 | 0.99 | TT | 1.02 | GT | 0.94 | GG | 0.86 |
| rs1432679 | 5 | A/G | 0.43 | 1.0670 | 1.06 | AA | 0.94 | GA | 1.01 | GG | 1.08 |
| rs11242675 | 6 | T/C | 0.39 | 0.9429 | 0.96 | TT | 1.05 | CT | 0.99 | CC | 0.93 |
| rs204247 | 6 | A/G | 0.43 | 1.0503 | 1.04 | AA | 0.96 | GA | 1.01 | GG | 1.06 |
| rs17529111 | 6 | A/G | 0.218 | 1.0457 | 1.02 | AA | 0.98 | GA | 1.03 | GG | 1.07 |
| rs12662670 | 6 | T/G | 0.073 | 1.1392 | 1.02 | TT | 0.98 | GT | 1.12 | GG | 1.27 |
| rs2046210 | 6 | G/A | 0.34 | 1.0471 | 1.03 | GG | 0.97 | AG | 1.01 | AA | 1.06 |
| rs720475 | 7 | G/A | 0.25 | 0.9452 | 0.97 | GG | 1.03 | AG | 0.97 | AA | 0.92 |
| rs9693444 | 8 | C/A | 0.32 | 1.0730 | 1.05 | CC | 0.95 | AC | 1.02 | AA | 1.10 |
| rs6472903 | 8 | T/G | 0.18 | 0.9124 | 0.97 | TT | 1.03 | GT | 0.94 | GG | 0.86 |
| rs2943559 | 8 | A/G | 0.07 | 1.1334 | 1.02 | AA | 0.98 | GA | 1.11 | GG | 1.26 |
| rs13281615 | 8 | A/G | 0.41 | 1.0950 | 1.08 | AA | 0.93 | GA | 1.01 | GG | 1.11 |
| rs11780156 | 8 | C/T | 0.16 | 1.0691 | 1.02 | CC | 0.98 | TC | 1.05 | TT | 1.12 |
| rs1011970 | 9 | G/T | 0.17 | 1.0502 | 1.02 | GG | 0.98 | TG | 1.03 | TT | 1.08 |
| rs10759243 | 9 | C/A | 0.39 | 1.0542 | 1.04 | CC | 0.96 | AC | 1.01 | AA | 1.07 |
| rs865686 | 9 | T/G | 0.38 | 0.8985 | 0.92 | TT | 1.08 | GT | 0.97 | GG | 0.87 |
| rs2380205 | 10 | C/T | 0.44 | 0.9771 | 0.98 | CC | 1.02 | TC | 1.00 | TT | 0.97 |
| rs7072776 | 10 | G/A | 0.29 | 1.0581 | 1.03 | GG | 0.97 | AG | 1.02 | AA | 1.08 |
| rs11814448 | 10 | A/C | 0.02 | 1.2180 | 1.01 | AA | 0.99 | CA | 1.21 | CC | 1.47 |
| rs10995190 | 10 | G/A | 0.16 | 0.8563 | 0.95 | GG | 1.05 | AG | 0.90 | AA | 0.77 |
| rs704010 | 10 | C/T | 0.38 | 1.0699 | 1.05 | CC | 0.95 | TC | 1.02 | TT | 1.09 |
| rs7904519 | 10 | A/G | 0.46 | 1.0584 | 1.05 | AA | 0.95 | GA | 1.00 | GG | 1.06 |
| rs2981579 | 10 | G/A | 0.4 | 1.2524 | 1.21 | GG | 0.83 | AG | 1.03 | AA | 1.29 |
| rs11199914 | 10 | C/T | 0.32 | 0.9400 | 0.96 | CC | 1.04 | TC | 0.98 | TT | 0.92 |
| rs3817198 | 11 | T/C | 0.31 | 1.0744 | 1.05 | TT | 0.96 | CT | 1.03 | CC | 1.10 |
| rs3903072 | 11 | G/T | 0.47 | 0.9442 | 0.95 | GG | 1.05 | TG | 1.00 | TT | 0.94 |
| rs554219 | 11 | C/G | 0.112 | 1.1238 | 1.03 | CC | 0.97 | GC | 1.09 | GG | 1.23 |
| rs78540526 | 11 | C/T | 0.032 | 1.1761 | 1.01 | CC | 0.99 | TC | 1.16 | TT | 1.37 |
| rs75915166 | 11 | C/A | 0.059 | 1.0239 | 1.00 | CC | 1.00 | AC | 1.02 | AA | 1.05 |
| rs11820646 | 11 | C/T | 0.41 | 0.9563 | 0.96 | CC | 1.04 | TC | 0.99 | TT | 0.95 |
| rs12422552 | 12 | G/C | 0.26 | 1.0327 | 1.02 | GG | 0.98 | CG | 1.02 | CC | 1.05 |
| rs10771399 | 12 | A/G | 0.12 | 0.8629 | 0.97 | AA | 1.03 | GA | 0.89 | GG | 0.77 |
| rs17356907 | 12 | A/G | 0.3 | 0.9078 | 0.95 | AA | 1.06 | GA | 0.96 | GG | 0.87 |
| rs1292011 | 12 | A/G | 0.42 | 0.9219 | 0.94 | AA | 1.07 | GA | 0.99 | GG | 0.91 |
| rs11571833 | 13 | A/T | 0.008 | 1.2609 | 1.00 | AA | 1.00 | TA | 1.26 | TT | 1.58 |
| rs2236007 | 14 | G/A | 0.21 | 0.9203 | 0.97 | GG | 1.03 | AG | 0.95 | AA | 0.88 |
| rs999737 | 14 | C/T | 0.23 | 0.9239 | 0.97 | CC | 1.04 | TC | 0.96 | TT | 0.88 |
| rs2588809 | 14 | C/T | 0.16 | 1.0667 | 1.02 | CC | 0.98 | TC | 1.04 | TT | 1.11 |
| rs941764 | 14 | A/G | 0.34 | 1.0636 | 1.04 | AA | 0.96 | GA | 1.02 | GG | 1.08 |
| rs3803662 | 16 | G/A | 0.26 | 1.2257 | 1.12 | GG | 0.89 | AG | 1.09 | AA | 1.34 |
| rs17817449 | 16 | T/G | 0.4 | 0.9300 | 0.94 | TT | 1.06 | GT | 0.98 | GG | 0.92 |
| rs11075995 | 16 | A/T | 0.241 | 1.0368 | 1.02 | AA | 0.98 | TA | 1.02 | TT | 1.06 |
| rs13329835 | 16 | A/G | 0.22 | 1.0758 | 1.03 | AA | 0.97 | GA | 1.04 | GG | 1.12 |
| rs6504950 | 17 | G/A | 0.28 | 0.9340 | 0.96 | GG | 1.04 | AG | 0.97 | AA | 0.91 |
| rs527616 | 18 | G/C | 0.38 | 0.9573 | 0.97 | GG | 1.03 | CG | 0.99 | CC | 0.95 |
| rs1436904 | 18 | T/G | 0.4 | 0.9466 | 0.96 | TT | 1.04 | GT | 0.99 | GG | 0.94 |
| rs2363956 | 19 | G/T | 0.487 | 1.0264 | 1.03 | GG | 0.97 | TG | 1.00 | TT | 1.03 |
| rs8170 | 19 | G/A | 0.19 | 1.0314 | 1.01 | GG | 0.99 | AG | 1.02 | AA | 1.05 |
| rs4808801 | 19 | A/G | 0.35 | 0.9349 | 0.95 | AA | 1.05 | GA | 0.98 | GG | 0.92 |
| rs3760982 | 19 | G/A | 0.46 | 1.0553 | 1.05 | GG | 0.95 | AG | 1.00 | AA | 1.06 |
| rs2823093 | 21 | G/A | 0.27 | 0.9274 | 0.96 | GG | 1.04 | AG | 0.96 | AA | 0.89 |
| rs17879961 | 22 | A/G | 0.005 | 1.3632 | 1.00 | AA | 1.00 | GA | 1.36 | GG | 1.85 |
| rs132390 | 22 | T/C | 0.036 | 1.1091 | 1.01 | TT | 0.99 | CT | 1.10 | CC | 1.22 |
| rs6001930 | 22 | T/C | 0.11 | 1.1345 | 1.03 | TT | 0.97 | CT | 1.10 | CC | 1.25 |

TABLE 12
Examples of African American polymorphisms (n = 78). Alleles represented
as risk/reference (non-risk) (e.g., for rs616488 A is the risk allele).

| | | | Risk | OR | | | | | | | |
|---|---|---|---|---|---|---|---|---|---|---|---|
| | | | allele | Risk | | | | | | | |
| Polymorphism | Chromosome | Alleles | frequency | Allele | μ | Adjusted Risk Score | | | | | |
| rs616488 | 1 | A/G | 0.86 | 1.03 | 1.05 | AA | 0.95 | AG | 0.98 | GG | 1.01 |
| rs11552449 | 1 | C/T | 0.037 | 0.9 | 0.99 | CC | 1.01 | CT | 0.91 | TT | 0.82 |
| rs11249433 | 1 | A/G | 0.13 | 0.99 | 1.00 | AA | 1.00 | AG | 0.99 | GG | 0.98 |
| rs6678914 | 1 | G/A | 0.66 | 1 | 1.00 | GG | 1.00 | GA | 1.00 | AA | 1.00 |
| rs4245739 | 1 | A/C | 0.24 | 0.97 | 0.99 | AA | 1.01 | AC | 0.98 | CC | 0.95 |
| rs12710696 | 2 | G/A | 0.53 | 1.06 | 1.06 | GG | 0.94 | GA | 1.00 | AA | 1.06 |
| rs4849887 | 2 | C/T | 0.7 | 1.16 | 1.24 | CC | 0.81 | CT | 0.94 | TT | 1.09 |
| rs2016394 | 2 | G/A | 0.72 | 1.05 | 1.07 | GG | 0.93 | GA | 0.98 | AA | 1.03 |
| rs1550623 | 2 | A/G | 0.71 | 1.1 | 1.15 | AA | 0.87 | AG | 0.96 | GG | 1.05 |
| rs1045485 | 2 | G/C | 0.93 | 0.99 | 0.98 | GG | 1.02 | GC | 1.01 | CC | 1.00 |
| rs13387042 | 2 | A/G | 0.72 | 1.12 | 1.18 | AA | 0.85 | AG | 0.95 | GG | 1.06 |
| rs16857609 | 2 | C/T | 0.24 | 1.17 | 1.08 | CC | 0.92 | CT | 1.08 | TT | 1.26 |
| rs6762644 | 3 | A/G | 0.46 | 1.05 | 1.05 | AA | 0.96 | AG | 1.00 | GG | 1.05 |
| rs4973768 | 3 | C/T | 0.36 | 1.04 | 1.03 | CC | 0.97 | CT | 1.01 | TT | 1.05 |
| rs12493607 | 3 | G/C | 0.14 | 1.04 | 1.01 | GG | 0.99 | GC | 1.03 | CC | 1.07 |
| rs9790517 | 4 | C/T | 0.084 | 0.88 | 0.98 | CC | 1.02 | CT | 0.90 | TT | 0.79 |
| rs6828523 | 4 | C/A | 0.65 | 1 | 1.00 | CC | 1.00 | CA | 1.00 | AA | 1.00 |
| rs4415084 | 5 | C/T | 0.61 | 1.1 | 1.13 | CC | 0.89 | CT | 0.98 | TT | 1.07 |
| rs10069690 | 5 | C/T | 0.57 | 1.13 | 1.15 | CC | 0.87 | CT | 0.98 | TT | 1.11 |
| rs10941679 | 5 | A/G | 0.21 | 1.04 | 1.02 | AA | 0.98 | AG | 1.02 | GG | 1.06 |
| rs889312 | 5 | A/C | 0.33 | 1.07 | 1.05 | AA | 0.96 | AC | 1.02 | CC | 1.09 |
| rs10472076 | 5 | T/C | 0.28 | 0.95 | 0.97 | TT | 1.03 | TC | 0.98 | CC | 0.93 |
| rs1353747 | 5 | T/G | 0.98 | 1.01 | 1.02 | TT | 0.98 | TG | 0.99 | GG | 1.00 |
| rs1432679 | 5 | A/G | 0.79 | 1.07 | 1.11 | AA | 0.90 | AG | 0.96 | GG | 1.03 |
| rs11242675 | 6 | T/C | 0.51 | 1.06 | 1.06 | TT | 0.94 | TC | 1.00 | CC | 1.06 |
| rs204247 | 6 | A/G | 0.34 | 1.13 | 1.09 | AA | 0.92 | AG | 1.04 | GG | 1.17 |
| rs17529111 | 6 | A/G | 0.075 | 0.99 | 1.00 | AA | 1.00 | AG | 0.99 | GG | 0.98 |
| rs9485370 | 6 | G/T | 0.78 | 1.13 | 1.21 | GG | 0.82 | GT | 0.93 | TT | 1.05 |
| rs3757318 | 6 | G/A | 0.038 | 1.11 | 1.01 | GG | 0.99 | GA | 1.10 | AA | 1.22 |
| rs2046210 | 6 | G/A | 0.6 | 0.99 | 0.99 | GG | 1.01 | GA | 1.00 | AA | 0.99 |
| rs720475 | 7 | G/A | 0.88 | 0.99 | 0.98 | GG | 1.02 | GA | 1.01 | AA | 1.00 |
| rs9693444 | 8 | C/A | 0.37 | 1.06 | 1.04 | CC | 0.96 | CA | 1.01 | AA | 1.08 |
| rs6472903 | 8 | T/G | 0.9 | 1.02 | 1.04 | TT | 0.96 | TG | 0.98 | GG | 1.00 |
| rs2943559 | 8 | A/G | 0.22 | 1.07 | 1.03 | AA | 0.97 | AG | 1.04 | GG | 1.11 |
| rs13281615 | 8 | A/G | 0.43 | 1.06 | 1.05 | AA | 0.95 | AG | 1.01 | GG | 1.07 |
| rs11780156 | 8 | C/T | 0.052 | 0.84 | 0.98 | CC | 1.02 | CT | 0.85 | TT | 0.72 |
| rs1011970 | 9 | G/T | 0.32 | 1.06 | 1.04 | GG | 0.96 | GT | 1.02 | TT | 1.08 |
| rs10759243 | 9 | C/A | 0.59 | 1.02 | 1.02 | CC | 0.98 | CA | 1.00 | AA | 1.02 |
| rs865686 | 9 | T/G | 0.51 | 1.09 | 1.09 | TT | 0.91 | TG | 1.00 | GG | 1.09 |
| rs2380205 | 10 | C/T | 0.42 | 0.98 | 0.98 | CC | 1.02 | CT | 1.00 | TT | 0.98 |
| rs7072776 | 10 | G/A | 0.49 | 1.04 | 1.04 | GG | 0.96 | GA | 1.00 | AA | 1.04 |
| rs11814448 | 10 | A/C | 0.61 | 1.04 | 1.05 | AA | 0.95 | AC | 0.99 | CC | 1.03 |
| rs10822013 | 10 | T/C | 0.23 | 1 | 1.00 | TT | 1.00 | TC | 1.00 | CC | 1.00 |
| rs10995190 | 10 | G/A | 0.83 | 0.98 | 0.97 | GG | 1.03 | GA | 1.01 | AA | 0.99 |
| rs704010 | 10 | C/T | 0.11 | 0.98 | 1.00 | CC | 1.00 | CT | 0.98 | TT | 0.96 |
| rs7904519 | 10 | A/G | 0.78 | 1.13 | 1.21 | AA | 0.82 | AG | 0.93 | GG | 1.05 |
| rs2981579 | 10 | G/A | 0.59 | 1.18 | 1.22 | GG | 0.82 | GA | 0.96 | AA | 1.14 |
| rs2981582 | 10 | G/A | 0.49 | 1.05 | 1.05 | GG | 0.95 | GA | 1.00 | AA | 1.05 |
| rs11199914 | 10 | C/T | 0.48 | 0.97 | 0.97 | CC | 1.03 | CT | 1.00 | TT | 0.97 |
| rs3817198 | 11 | T/C | 0.17 | 0.98 | 0.99 | TT | 1.01 | TC | 0.99 | CC | 0.97 |
| rs3903072 | 11 | G/T | 0.82 | 0.99 | 0.98 | GG | 1.02 | GT | 1.01 | TT | 1.00 |
| rs554219 | 11 | C/G | 0.22 | 1 | 1.00 | CC | 1.00 | CG | 1.00 | GG | 1.00 |
| rs614367 | 11 | G/A | 0.13 | 0.96 | 0.99 | GG | 1.01 | GA | 0.97 | AA | 0.93 |
| rs75915166 | 11 | C/A | 0.015 | 1.44 | 1.01 | CC | 0.99 | CA | 1.42 | AA | 2.05 |
| rs11820646 | 11 | C/T | 0.78 | 0.98 | 0.97 | CC | 1.03 | CT | 1.01 | TT | 0.99 |
| rs12422552 | 12 | G/C | 0.41 | 1.02 | 1.02 | GG | 0.98 | GC | 1.00 | CC | 1.02 |
| rs10771399 | 12 | A/G | 0.96 | 1.19 | 1.40 | AA | 0.72 | AG | 0.85 | GG | 1.01 |
| rs17356907 | 12 | A/G | 0.79 | 1.02 | 1.03 | AA | 0.97 | AG | 0.99 | GG | 1.01 |
| rs1292011 | 12 | A/G | 0.55 | 1.03 | 1.03 | AA | 0.97 | AG | 1.00 | GG | 1.03 |
| rs11571833 | 13 | A/T | 0.003 | 0.95 | 1.00 | AA | 1.00 | AT | 0.95 | TT | 0.90 |
| rs2236007 | 14 | G/A | 0.93 | 0.9 | 0.82 | GG | 1.22 | GA | 1.09 | AA | 0.98 |
| rs999737 | 14 | C/T | 0.95 | 1.03 | 1.06 | CC | 0.95 | CT | 0.97 | TT | 1.00 |
| rs2588809 | 14 | C/T | 0.29 | 1.01 | 1.01 | CC | 0.99 | CT | 1.00 | TT | 1.01 |
| rs941764 | 14 | A/G | 0.7 | 1.1 | 1.14 | AA | 0.87 | AG | 0.96 | GG | 1.06 |
| rs3803662 | 16 | G/A | 0.51 | 0.99 | 0.99 | GG | 1.01 | GA | 1.00 | AA | 0.99 |
| rs17817449 | 16 | T/G | 0.6 | 1.05 | 1.06 | TT | 0.94 | TG | 0.99 | GG | 1.04 |
| rs11075995 | 16 | A/T | 0.18 | 1.07 | 1.03 | AA | 0.98 | AT | 1.04 | TT | 1.12 |
| rs13329835 | 16 | A/G | 0.63 | 1.08 | 1.10 | AA | 0.91 | AG | 0.98 | GG | 1.06 |
| rs6504950 | 17 | G/A | 0.65 | 1.06 | 1.08 | GG | 0.93 | GA | 0.98 | AA | 1.04 |
| rs527616 | 18 | G/C | 0.86 | 0.98 | 0.97 | GG | 1.04 | GC | 1.01 | CC | 0.99 |
| rs1436904 | 18 | T/G | 0.75 | 0.98 | 0.97 | TT | 1.03 | TG | 1.01 | GG | 0.99 |
| rs8170 | 19 | G/A | 0.19 | 1.13 | 1.05 | GG | 0.95 | GA | 1.08 | AA | 1.22 |
| rs4808801 | 19 | A/G | 0.33 | 1.01 | 1.01 | AA | 0.99 | AG | 1.00 | GG | 1.01 |
| rs3760982 | 19 | G/A | 0.47 | 1 | 1.00 | GG | 1.00 | GA | 1.00 | AA | 1.00 |
| rs2284378 | 20 | C/T | 0.16 | 1.06 | 1.02 | CC | 0.98 | CT | 1.04 | TT | 1.10 |
| rs2823093 | 21 | G/A | 0.57 | 1.03 | 1.03 | GG | 0.97 | GA | 1.00 | AA | 1.03 |
| rs132390 | 22 | T/C | 0.052 | 0.88 | 0.99 | TT | 1.01 | TC | 0.89 | CC | 0.78 |
| rs6001930 | 22 | T/C | 0.13 | 1.02 | 1.01 | TT | 0.99 | TC | 1.01 | CC | 1.04 |

TABLE 13
Examples of Hispanic polymorphisms (n = 82). Alleles represented as major/minor (e.g. for
rs616488 A is the common allele and G less common). Minor allele odds ratios below 1 means the
minor allele is not the risk allele, whereas when above 1 the minor allele is the risk allele.

| | | | Minor | OR | | | | | | | |
|---|---|---|---|---|---|---|---|---|---|---|---|
| | | | allele | Minor | | | | | | | |
| Polymorphism | Chromosome | Alleles | frequency | Allele | μ | Adjusted Risk Score | | | | | |
| rs616488 | 1 | A/G | 0.33 | 0.9417 | 0.96 | AA | 1.04 | GA | 0.98 | GG | 0.92 |
| rs11552449 | 1 | C/T | 0.17 | 1.0810 | 1.03 | CC | 0.97 | TC | 1.05 | TT | 1.14 |
| rs11249433 | 1 | A/G | 0.40 | 1.0993 | 1.08 | AA | 0.93 | GA | 1.02 | GG | 1.12 |
| rs6678914 | 1 | G/A | 0.414 | 0.9890 | 0.99 | GG | 1.01 | AG | 1.00 | AA | 0.99 |
| rs4245739 | 1 | A/C | 0.258 | 1.0291 | 1.02 | AA | 0.99 | CA | 1.01 | CC | 1.04 |
| rs12710696 | 2 | G/A | 0.357 | 1.0387 | 1.03 | GG | 0.97 | AG | 1.01 | AA | 1.05 |
| rs4849887 | 2 | C/T | 0.098 | 0.9187 | 0.98 | CC | 1.02 | TC | 0.93 | TT | 0.86 |
| rs2016394 | 2 | G/A | 0.48 | 0.9504 | 0.95 | GG | 1.05 | AG | 1.00 | AA | 0.95 |
| rs1550623 | 2 | A/G | 0.16 | 0.9445 | 0.98 | AA | 1.02 | GA | 0.96 | GG | 0.91 |
| rs1045485 | 2 | G/C | 0.13 | 0.9644 | 0.99 | GG | 1.01 | CG | 0.97 | CC | 0.94 |
| rs13387042 | 2 | A/G | 0.49 | 0.8794 | 0.89 | AA | 1.13 | GA | 0.99 | GG | 0.87 |
| rs16857609 | 2 | C/T | 0.26 | 1.0721 | 1.04 | CC | 0.96 | TC | 1.03 | TT | 1.11 |
| rs6762644 | 3 | A/G | 0.4 | 1.066 | 1.05 | AA | 0.95 | GA | 1.01 | GG | 1.08 |
| rs4973768 | 3 | C/T | 0.47 | 1.0938 | 1.09 | CC | 0.92 | TC | 1.00 | TT | 1.10 |
| rs12493607 | 3 | G/C | 0.35 | 1.0529 | 1.04 | GG | 0.96 | CG | 1.01 | CC | 1.07 |
| rs7696175 | 4 | T/C | 0.38 | 1.14 | 1.11 | TT | 0.90 | CT | 1.03 | CC | 1.17 |
| rs9790517 | 4 | C/T | 0.23 | 1.0481 | 1.02 | CC | 0.98 | TC | 1.03 | TT | 1.07 |
| rs6828523 | 4 | C/A | 0.13 | 0.9056 | 0.98 | CC | 1.03 | AC | 0.93 | AA | 0.84 |
| rs10069690 | 5 | C/T | 0.26 | 1.0242 | 1.01 | CC | 0.99 | TC | 1.01 | TT | 1.04 |
| rs7726159 | 5 | C/A | 0.338 | 1.0359 | 1.02 | CC | 0.98 | AC | 1.01 | AA | 1.05 |
| rs2736108 | 5 | C/T | 0.292 | 0.9379 | 0.96 | CC | 1.04 | TC | 0.97 | TT | 0.91 |
| rs10941679 | 5 | A/G | 0.25 | 1.1198 | 1.06 | AA | 0.94 | GA | 1.06 | GG | 1.18 |
| rs889312 | 5 | A/C | 0.28 | 1.1176 | 1.07 | AA | 0.94 | CA | 1.05 | CC | 1.17 |
| rs10472076 | 5 | T/C | 0.38 | 1.0419 | 1.03 | TT | 0.97 | CT | 1.01 | CC | 1.05 |
| rs2067980 | 5 | G/A | 0.16 | 1 | 1.00 | GG | 1.00 | AG | 1.00 | AA | 1.00 |
| rs1353747 | 5 | T/G | 0.095 | 0.9213 | 0.99 | TT | 1.02 | GT | 0.94 | GG | 0.86 |
| rs1432679 | 5 | A/G | 0.43 | 1.0670 | 1.06 | AA | 0.94 | GA | 1.01 | GG | 1.08 |
| rs11242675 | 6 | T/C | 0.39 | 0.9429 | 0.96 | TT | 1.05 | CT | 0.99 | CC | 0.93 |
| rs204247 | 6 | A/G | 0.43 | 1.0503 | 1.04 | AA | 0.96 | GA | 1.01 | GG | 1.06 |
| rs17529111 | 6 | A/G | 0.218 | 1.0457 | 1.02 | AA | 0.98 | GA | 1.03 | GG | 1.07 |
| rs2180341 | 6 | G/A | 0.23 | 0.9600 | 0.98 | GG | 1.02 | AG | 0.98 | AA | 0.94 |
| rs12662670 | 6 | T/G | 0.073 | 1.1392 | 1.02 | TT | 0.98 | GT | 1.12 | GG | 1.27 |
| rs2046210 | 6 | G/A | 0.34 | 1.0471 | 1.03 | GG | 0.97 | AG | 1.01 | AA | 1.06 |
| rs17157903 | 7 | T/C | 0.09 | 0.93 | 0.99 | TT | 1.01 | CT | 0.94 | CC | 0.88 |
| rs720475 | 7 | G/A | 0.25 | 0.9452 | 0.97 | GG | 1.03 | AG | 0.97 | AA | 0.92 |
| rs9693444 | 8 | C/A | 0.32 | 1.0730 | 1.05 | CC | 0.95 | AC | 1.02 | AA | 1.10 |
| rs6472903 | 8 | T/G | 0.18 | 0.9124 | 0.97 | TT | 1.03 | GT | 0.94 | GG | 0.86 |
| rs2943559 | 8 | A/G | 0.07 | 1.1334 | 1.02 | AA | 0.98 | GA | 1.11 | GG | 1.26 |
| rs13281615 | 8 | A/G | 0.41 | 1.0950 | 1.08 | AA | 0.93 | GA | 1.01 | GG | 1.11 |
| rs11780156 | 8 | C/T | 0.16 | 1.0691 | 1.02 | CC | 0.98 | TC | 1.05 | TT | 1.12 |
| rs1011970 | 9 | G/T | 0.17 | 1.0502 | 1.02 | GG | 0.98 | TG | 1.03 | TT | 1.08 |
| rs10759243 | 9 | C/A | 0.39 | 1.0542 | 1.04 | CC | 0.96 | AC | 1.01 | AA | 1.07 |
| rs865686 | 9 | T/G | 0.38 | 0.8985 | 0.92 | TT | 1.08 | GT | 0.97 | GG | 0.87 |
| rs2380205 | 10 | C/T | 0.44 | 0.9771 | 0.98 | CC | 1.02 | TC | 1.00 | TT | 0.97 |
| rs7072776 | 10 | G/A | 0.29 | 1.0581 | 1.03 | GG | 0.97 | AG | 1.02 | AA | 1.08 |
| rs11814448 | 10 | A/C | 0.02 | 1.2180 | 1.01 | AA | 0.99 | CA | 1.21 | CC | 1.47 |
| rs10995190 | 10 | G/A | 0.16 | 0.8563 | 0.95 | GG | 1.05 | AG | 0.90 | AA | 0.77 |
| rs704010 | 10 | C/T | 0.38 | 1.0699 | 1.05 | CC | 0.95 | TC | 1.02 | TT | 1.09 |
| rs7904519 | 10 | A/G | 0.46 | 1.0584 | 1.05 | AA | 0.95 | GA | 1.00 | GG | 1.06 |
| rs2981579 | 10 | G/A | 0.4 | 1.2524 | 1.21 | GG | 0.83 | AG | 1.03 | AA | 1.29 |
| rs2981582 | 10 | T/C | 0.42 | 1.1900 | 1.17 | TT | 0.86 | CT | 1.02 | CC | 1.21 |
| rs11199914 | 10 | C/T | 0.32 | 0.9400 | 0.96 | CC | 1.04 | TC | 0.98 | TT | 0.92 |
| rs3817198 | 11 | T/C | 0.31 | 1.0744 | 1.05 | TT | 0.96 | CT | 1.03 | CC | 1.10 |
| rs3903072 | 11 | G/T | 0.47 | 0.9442 | 0.95 | GG | 1.05 | TG | 1.00 | TT | 0.94 |
| rs554219 | 11 | C/G | 0.112 | 1.1238 | 1.03 | CC | 0.97 | GC | 1.09 | GG | 1.23 |
| rs78540526 | 11 | C/T | 0.032 | 1.1761 | 1.01 | CC | 0.99 | TC | 1.16 | TT | 1.37 |
| rs75915166 | 11 | C/A | 0.059 | 1.0239 | 1.00 | CC | 1.00 | AC | 1.02 | AA | 1.05 |
| rs11820646 | 11 | C/T | 0.41 | 0.9563 | 0.96 | CC | 1.04 | TC | 0.99 | TT | 0.95 |
| rs12422552 | 12 | G/C | 0.26 | 1.0327 | 1.02 | GG | 0.98 | CG | 1.02 | CC | 1.05 |
| rs10771399 | 12 | A/G | 0.12 | 0.8629 | 0.97 | AA | 1.03 | GA | 0.89 | GG | 0.77 |
| rs17356907 | 12 | A/G | 0.3 | 0.9078 | 0.95 | AA | 1.06 | GA | 0.96 | GG | 0.87 |
| rs1292011 | 12 | A/G | 0.42 | 0.9219 | 0.94 | AA | 1.07 | GA | 0.99 | GG | 0.91 |
| rs11571833 | 13 | A/T | 0.008 | 1.2609 | 1.00 | AA | 1.00 | TA | 1.26 | TT | 1.58 |
| rs2236007 | 14 | G/A | 0.21 | 0.9203 | 0.97 | GG | 1.03 | AG | 0.95 | AA | 0.88 |
| rs999737 | 14 | C/T | 0.23 | 0.9239 | 0.97 | CC | 1.04 | TC | 0.96 | TT | 0.88 |
| rs2588809 | 14 | C/T | 0.16 | 1.0667 | 1.02 | CC | 0.98 | TC | 1.04 | TT | 1.11 |
| rs941764 | 14 | A/G | 0.34 | 1.0636 | 1.04 | AA | 0.96 | GA | 1.02 | GG | 1.08 |
| rs3803662 | 16 | G/A | 0.26 | 1.2257 | 1.12 | GG | 0.89 | AG | 1.09 | AA | 1.34 |
| rs17817449 | 16 | T/G | 0.4 | 0.9300 | 0.94 | TT | 1.06 | GT | 0.98 | GG | 0.92 |
| rs11075995 | 16 | A/T | 0.241 | 1.0368 | 1.02 | AA | 0.98 | TA | 1.02 | TT | 1.06 |
| rs13329835 | 16 | A/G | 0.22 | 1.0758 | 1.03 | AA | 0.97 | GA | 1.04 | GG | 1.12 |
| rs6504950 | 17 | G/A | 0.28 | 0.9340 | 0.96 | GG | 1.04 | AG | 0.97 | AA | 0.91 |
| rs527616 | 18 | G/C | 0.38 | 0.9573 | 0.97 | GG | 1.03 | CG | 0.99 | CC | 0.95 |
| rs1436904 | 18 | T/G | 0.4 | 0.9466 | 0.96 | TT | 1.04 | GT | 0.99 | GG | 0.94 |
| rs2363956 | 19 | G/T | 0.487 | 1.0264 | 1.03 | GG | 0.97 | TG | 1.00 | TT | 1.03 |
| rs8170 | 19 | G/A | 0.19 | 1.0314 | 1.01 | GG | 0.99 | AG | 1.02 | AA | 1.05 |
| rs4808801 | 19 | A/G | 0.35 | 0.9349 | 0.95 | AA | 1.05 | GA | 0.98 | GG | 0.92 |
| rs3760982 | 19 | G/A | 0.46 | 1.0553 | 1.05 | GG | 0.95 | AG | 1.00 | AA | 1.06 |
| rs2823093 | 21 | G/A | 0.27 | 0.9274 | 0.96 | GG | 1.04 | AG | 0.96 | AA | 0.89 |
| rs17879961 | 22 | A/G | 0.005 | 1.3632 | 1.00 | AA | 1.00 | GA | 1.36 | GG | 1.85 |
| rs132390 | 22 | T/C | 0.036 | 1.1091 | 1.01 | TT | 0.99 | CT | 1.10 | CC | 1.22 |
| rs6001930 | 22 | T/C | 0.11 | 1.1345 | 1.03 | TT | 0.97 | CT | 1.10 | CC | 1.25 |

TABLE 14
Expanded list of polymorphisms indicative
of breast cancer risk (n = 203).

| | | Major | Minor | Odds ratio |
|---|---|---|---|---|
| | | allele/minor | allele | minor |
| Polymorphism | Location | allele | frequency | allele |
| 12:120832146:C:T | 12q24.31 | C/T | 0.16 | 1.05 |
| 12:85009437:T:C | 12q21.31 | T/C | 0.34 | 0.95 |
| 17:44252468:G:A | 17q21.31 | G/A | 0.19 | 0.95 |
| 4:84370124 | 4q21.23 | TA/TAA | 0.47 | 1.04 |
| chr17:29230520 | 17q11.2 | GGT/G | 0.27 | 0.97 |
| chr22:39359355 | 22q13.1 | I/D**** | 0.10 | 1.10 |
| rs10022462 | 4q22.1 | C/T | 0.44 | 1.04 |
| rs10069690 | 5p15.33 | C/T | 0.26 | 1.06 |
| rs1011970 | 9p21.3 | G/T | 0.16 | 1.07 |
| rs10472076 | 5q11.2 | T/C | 0.38 | 1.03 |
| rs10474352 | 5q14.3 | C/T | 0.16 | 0.94 |
| rs1053338 | 3p14.1 | A/G | 0.14 | 1.05 |
| rs10623258 | 14q32.33 | C/CTT | 0.45 | 1.04 |
| rs10759243 | 9q31.2 | C/A | 0.29 | 1.06 |
| rs10760444 | 9q33.3 | A/G | 0.43 | 1.03 |
| rs10816625 | 9q31.2 | A/G | 0.06 | 1.11 |
| rs10941679 | 5p12 | A/G | 0.25 | 1.15 |
| rs10995201 | 10q21.2 | A/G | 0.16 | 0.90 |
| rs11075995 | 16q12.2 | T/A | 0.24 | 1.03 |
| rs11076805 | 16p13.3 | C/A | 0.26 | 0.97 |
| rs11117758 | 1q41 | G/A | 0.21 | 0.95 |
| rs11199914 | 10q26.12 | C/T | 0.32 | 0.96 |
| rs11242675 | 6p25.3 | T/C | 0.37 | 1.00 |
| rs11249433 | 1p11.2 | A/G | 0.41 | 1.11 |
| rs113577745 | 2p25.1 | C/G | 0.10 | 1.08 |
| rs113701136 | 19q12 | C/T | 0.32 | 1.03 |
| rs11374964 | 11q22.3 | G/GA | 0.42 | 1.00 |
| rs11389348 | 20q12 | G/GT | 0.38 | 0.96 |
| rs11552449 | 1p13.2 | C/T | 0.17 | 1.04 |
| rs11571833 | 13q13.1 | A/T | 0.01 | 1.35 |
| rs116095464 | 5p15.33 | T/C | 0.05 | 1.06 |
| rs11627032 | 14q32.12 | T/C | 0.25 | 0.96 |
| rs117618124 | 18q12.1 | T/C | 0.05 | 0.89 |
| rs11780156 | 8q24.21 | C/T | 0.17 | 1.05 |
| rs11814448 | 10p12.31 | A/C | 0.02 | 1.12 |
| rs11820646 | 11q24.3 | C/T | 0.40 | 0.96 |
| rs11977670 | 7q34 | G/A | 0.43 | 1.06 |
| rs12048493 | 1q21.2 | A/C | 0.38 | 1.04 |
| rs12207986 | 6q14.1 | A/G | 0.47 | 0.97 |
| rs12405132 | 1q21.1 | C/T | 0.37 | 0.97 |
| rs12422552 | 12p13.1 | G/C | 0.26 | 1.06 |
| rs12479355 | 2q36.3 | A/G | 0.21 | 0.96 |
| rs12493607 | 3p24.1 | G/C | 0.34 | 1.05 |
| rs12546444 | 8q23.1 | A/T | 0.10 | 0.93 |
| rs12624860 | 20q13.2 | C/G | 0.24 | 1.04 |
| rs12710696 | 2p24.1 | C/T | 0.37 | 1.03 |
| rs1292011 | 12q24.21 | A/G | 0.42 | 0.92 |
| rs13066793 | 3p12.1 | A/G | 0.09 | 0.94 |
| rs13162653 | 5p15.1 | G/T | 0.45 | 0.99 |
| rs132390 | 22q12.2 | T/C | 0.04 | 1.04 |
| rs13267382 | 8q23.3 | G/A | 0.36 | 1.03 |
| rs13281615 | 8q24.21 | A/G | 0.41 | 1.11 |
| rs13294895 | 9q31.2 | C/T | 0.18 | 1.06 |
| rs13329835 | 16q23.2 | A/G | 0.23 | 1.07 |
| rs13365225 | 8p11.23 | A/G | 0.18 | 0.91 |
| rs1353747 | 5q11.2 | T/G | 0.09 | 0.96 |
| rs140850326 | 1p32.3 | I/D* | 0.49 | 0.97 |
| rs140936696 | 10q23.33 | C/CAA | 0.18 | 1.04 |
| rs1432679 | 5q33.3 | T/C | 0.43 | 1.08 |
| rs1436904 | 18q11.2 | T/G | 0.40 | 0.95 |
| rs151090251 | 15q22.33 | D/I*** | 0.05 | 1.08 |
| rs1550623 | 2q31.1 | A/G | 0.15 | 0.95 |
| rs16857609 | 2q35 | C/T | 0.26 | 1.06 |
| rs16991615 | 20p12.3 | G/A | 0.06 | 1.10 |
| rs1707302 | 1p34.1 | G/A | 0.34 | 0.96 |
| rs17156577 | 7p15.1 | T/C | 0.11 | 1.05 |
| rs17268829 | 7q21.3 | T/C | 0.28 | 1.05 |
| rs17356907 | 12q22 | A/G | 0.30 | 0.91 |
| rs17426269 | 1p22.3 | G/A | 0.15 | 1.05 |
| rs17529111 | 6q14.1 | T/C | 0.22 | 1.02 |
| rs17817449 | 16q12.2 | T/G | 0.41 | 0.95 |
| rs17879961 | 22q12.1 | A/G | 0.005 | 1.26 |
| rs1830298 | 2q33.1 | T/C | 0.28 | 1.06 |
| rs1895062 | 9q33.1 | A/G | 0.41 | 0.94 |
| rs2012709 | 5p13.3 | C/T | 0.48 | 1.02 |
| rs2016394 | 2q31.1 | G/A | 0.47 | 0.95 |
| rs204247 | 6p23 | A/G | 0.44 | 1.04 |
| rs2223621 | 6p22.3 | C/T | 0.38 | 1.04 |
| rs2236007 | 14q13.3 | G/A | 0.21 | 0.93 |
| rs2284378 | 20q11.22 | C/T | 0.32 | 1.00 |
| rs2290203 | 15q26.1 | G/A | 0.21 | 0.94 |
| rs2380205 | 10p15.1 | C/T | 0.44 | 0.98 |
| rs2432539 | 16q13 | G/A | 0.40 | 1.03 |
| rs2588809 | 14q24.1 | C/T | 0.17 | 1.06 |
| rs2594714 | 19p13.12 | G/A | 0.23 | 0.97 |
| rs2747652 | 6q25 | C/T | 0.48 | 0.94 |
| rs2787486 | 17q22 | A/C | 0.30 | 0.93 |
| rs2823093 | 21q21.1 | G/A | 0.27 | 0.94 |
| rs28512361 | 22q13.31 | G/A | 0.11 | 1.05 |
| rs28539243 | 16q12.2 | G/A | 0.49 | 1.05 |
| rs2943559 | 8q21.11 | A/G | 0.08 | 1.10 |
| rs2965183 | 19p13.11 | G/A | 0.35 | 1.04 |
| rs2981578 | 10q26.13 | T/C | 0.47 | 1.23 |
| rs2992756 | 1p36.13 | C/T | 0.49 | 1.06 |
| rs310302 | 8p21.2 | G/A | 0.40 | 1.04 |
| rs3215401 | 5p15.33 | A/AG | 0.31 | 0.93 |
| rs322144 | 19p13.2 | C/G | 0.47 | 0.98 |
| rs34005590 | 2q35 | C/A | 0.05 | 0.82 |
| rs34207738 | 3q23 | CTT/C | 0.41 | 1.06 |
| rs35054928 | 10q26.13 | G/GC | 0.40 | 1.27 |
| rs35383942 | 1q32.1 | C/T | 0.06 | 1.12 |
| rs35951924 | 5q11.1 | A/AT | 0.32 | 0.95 |
| rs36194942 | 18q12.1 | A/AT | 0.30 | 0.98 |
| rs3757322 | 6q25 | T/G | 0.32 | 1.08 |
| rs3760982 | 19q13.31 | G/A | 0.46 | 1.05 |
| rs3817198 | 11p15.5 | T/C | 0.32 | 1.05 |
| rs3819405 | 6p22.3 | C/T | 0.33 | 0.96 |
| rs3903072 | 11q13.1 | G/T | 0.47 | 0.97 |
| rs4233486 | 1p34.2 | T/C | 0.36 | 0.97 |
| rs4245739 | 1q32.1 | A/C | 0.26 | 1.02 |
| rs4442975 | 2q35 | G/T | 0.50 | 0.89 |
| rs4496150 | 16q24.2 | C/A | 0.25 | 0.96 |
| rs4562056 | 5q35.1 | G/T | 0.33 | 1.05 |
| rs45631563 | 10q26.13 | A/T | 0.05 | 0.81 |
| rs4577244 | 2p23.2 | C/T | 0.23 | 1.01 |
| rs4593472 | 7q32.3 | C/T | 0.35 | 0.97 |
| rs4784227 | 16q12.1 | C/T | 0.24 | 1.23 |
| rs4808801 | 19p13.11 | A/G | 0.34 | 0.93 |
| rs4849887 | 2q14.1 | C/T | 0.10 | 0.91 |
| rs4951011 | 1q32.1 | A/G | 0.16 | 1.04 |
| rs4971059 | 1q22 | G/A | 0.35 | 1.05 |
| rs4973768 | 3p24.1 | C/T | 0.50 | 1.11 |
| rs514192 | 8q22.3 | T/A | 0.32 | 1.05 |
| rs527616 | 18q11.2 | G/C | 0.38 | 0.97 |
| rs554219 | 11q13.3 | C/G | 0.13 | 1.21 |
| rs58058861 | 3q26.31 | G/A | 0.21 | 1.06 |
| rs58847541 | 8q24.13 | G/A | 0.15 | 1.08 |
| rs6001930 | 22q13.1 | T/C | 0.10 | 1.12 |
| rs6062356 | 20q13.33 | T/G | 0.15 | 1.06 |
| rs6122906 | 20q13.13 | A/G | 0.18 | 1.05 |
| rs616488 | 1p36.22 | A/G | 0.33 | 0.94 |
| rs62355902 | 5q11.2 | A/T | 0.16 | 1.18 |
| rs6472903 | 8q21.11 | T/G | 0.17 | 0.94 |
| rs6507583 | 18q12.3 | A/G | 0.07 | 0.92 |
| rs6562760 | 13q22.1 | G/A | 0.24 | 0.95 |
| rs6569648 | 6q23.1 | T/C | 0.24 | 0.94 |
| rs6596100 | 5q31.1 | C/T | 0.25 | 0.94 |
| rs6597981 | 11p15 | G/A | 0.48 | 0.96 |
| rs6678914 | 1q32.1 | G/A | 0.41 | 1.00 |
| rs66823261 | 8p23.3 | T/C | 0.22 | 1.03 |
| rs6725517 | 2p23.3 | A/G | 0.41 | 0.96 |
| rs67397200 | 19p13.11 | C/G | 0.30 | 1.03 |
| rs676256 | 9q31.2 | T/C | 0.38 | 0.91 |
| rs6762644 | 3p26.1 | A/G | 0.38 | 1.05 |
| rs67958007 | 10p14 | TG/T | 0.12 | 0.19 |
| rs6796502 | 3p21.31 | G/A | 0.10 | 0.92 |
| rs6805189 | 3p13 | T/C | 0.48 | 0.97 |
| rs6815814 | 4p14 | A/C | 0.26 | 1.06 |
| rs6828523 | 4q34.1 | C/A | 0.12 | 0.91 |
| rs6882649 | 5q22.1 | T/G | 0.34 | 0.97 |
| rs6964587 | 7q21.2 | G/T | 0.39 | 1.03 |
| rs704010 | 10q22.3 | C/T | 0.38 | 1.07 |
| rs7072776 | 10p12.31 | G/A | 0.29 | 1.05 |
| rs71338792 | 19q13.22 | A/AT | 0.23 | 1.05 |
| rs71557345 | 6p22.2 | G/A | 0.07 | 0.92 |
| rs71559437 | 7q22.1 | G/A | 0.12 | 0.93 |
| rs71801447 | 2q13 | CTTATGTT/C | 0.06 | 1.09 |
| rs720475 | 7q35 | G/A | 0.25 | 0.96 |
| rs72749841 | 5q11.1 | T/C | 0.16 | 0.93 |
| rs72755295 | 1q43 | A/G | 0.03 | 1.05 |
| rs72826962 | 17q21.2 | C/T | 0.01 | 1.20 |
| rs7297051 | 12p11.22 | C/T | 0.24 | 0.89 |
| rs73161324 | 22q13.2 | C/T | 0.06 | 1.06 |
| rs738321 | 22q13.1 | C/G | 0.38 | 0.95 |
| rs745570 | 17q25.3 | G/A | 0.50 | 1.03 |
| rs74911261 | 11q22.3 | G/A | 0.03 | 1.01 |
| rs7529522 | 1p12 | T/C | 0.23 | 1.06 |
| rs75915166 | 11q13.3 | C/A | 0.06 | 1.28 |
| rs7707921 | 5q14.2 | A/T | 0.25 | 0.96 |
| rs77528541 | 4q28.1 | G/T | 0.13 | 0.95 |
| rs78269692 | 19p13.13 | T/C | 0.05 | 1.09 |
| rs7904519 | 10q25.2 | A/G | 0.46 | 1.03 |
| rs7971 | 7p15.3 | A/G | 0.35 | 0.96 |
| rs79724016 | 1p34.2 | T/G | 0.03 | 0.93 |
| rs8176636 | 9q34.2 | I/D** | 0.20 | 1.03 |
| rs9257408 | 6p22.1 | G/C | 0.41 | 1.02 |
| rs9348512 | 6p24.3 | C/A | 0.33 | 1.00 |
| rs9358466 | 6p22.3 | T/C | 0.43 | 0.97 |
| rs9397437 | 6q25 | G/A | 0.07 | 1.17 |
| rs941764 | 14q32.11 | A/G | 0.35 | 1.03 |
| rs9485372 | 6q25.1 | G/A | 0.19 | 0.96 |
| rs9693444 | 8p12 | C/A | 0.32 | 1.06 |
| rs9790517 | 4q24 | C/T | 0.23 | 1.04 |
| rs9833888 | 3p12.1 | G/T | 0.22 | 1.06 |
| rs999737 | 14q24.1 | C/T | 0.23 | 0.91 |
| rs2981582 | 10q26 | T/C | | |
| rs3803662 | 16q12.1 | G/A | | |
| rs889312 | 5q11.2 | A/C | | |
| rs13387042 | 2q35 | A/G | | |
| rs4415084 | 5p12 | C/T | | |
| rs1045485 | 2q33.1 | G/C | | |
| rs2736108 | 5p15.33 | C/T | | |
| rs7726159 | 5p15.33 | C/A | | |
| rs12662670 | 6q25.1 | T/G | | |
| rs2046210 | 6q25.1 | G/A | | |
| rs865686 | 9q31.2 | T/G | | |
| rs2981579 | 10q26.13 | G/A | | |
| rs10995190 | 10q21.2 | G/A | | |
| rs78540526 | 11q13.3 | C/T | | |
| rs10771399 | 12p11.22 | A/G | | |
| rs6504950 | 17q22 | G/A | | |
| rs2363956 | 19p13 | G/T | | |
| rs8170 | 19p13.11 | G/A | | |

“*” indicates a 21 base pair deletion, “**” indicates a 36 base pair deletion, “***” indicates a 14 base pair insertion, and “****” indicates a 31 kb deletion. “Odds Ratio minor allele” values below 1 means that the minor allele is not the risk allele, whereas when above 1 the minor allele is the risk allele.

TABLE 15
Optimised list of African American polymorphisms (n = 74).

| Polymorphism | Chromosome | Major allele/minor allele |
|---|---|---|
| rs11249433 | 1 | G/A |
| rs4245739 | 1 | C/A |
| rs616488 | 1 | A/G |
| rs6678914 | 1 | G/A |
| rs1045485 | 2 | C/G |
| rs12710696 | 2 | A/G |
| rs13387042 | 2 | A/G |
| rs1550623 | 2 | A/G |
| rs16857609 | 2 | A/G |
| rs2016394 | 2 | G/A |
| rs4849887 | 2 | G/A |
| rs12493607 | 3 | G/C |
| rs4973768 | 3 | A/G |
| rs6762644 | 3 | G/A |
| rs6828523 | 4 | C/A |
| rs9790517 | 4 | A/G |
| rs10069690 | 5 | A/G |
| rs10472076 | 5 | G/A |
| rs10941679 | 5 | G/A |
| rs1353747 | 5 | A/C |
| rs1432679 | 5 | G/A |
| rs4415084 | 5 | T/C |
| rs889312 | 5 | C/A |
| rs11242675 | 6 | A/G |
| rs17529111 | 6 | G/A |
| rs204247 | 6 | G/A |
| rs2046210 | 6 | A/G |
| rs3757318 | 6 | A/G |
| rs720475 | 7 | G/A |
| rs11780156 | 8 | A/G |
| rs13281615 | 8 | G/A |
| rs2943559 | 8 | G/A |
| rs6472903 | 8 | A/C |
| rs9693444 | 8 | A/C |
| rs1011970 | 9 | A/C |
| rs10759243 | 9 | A/C |
| rs865686 | 9 | A/C |
| rs10995190 | 10 | G/A |
| rs11199914 | 10 | G/A |
| rs11814448 | 10 | C/A |
| rs2380205 | 10 | G/A |
| rs2981579 | 10 | A/G |
| rs2981582 | 10 | A/G |
| rs704010 | 10 | A/G |
| rs7072776 | 10 | A/G |
| rs7904519 | 10 | G/A |
| rs11820646 | 11 | G/A |
| rs3817198 | 11 | G/A |
| rs3903072 | 11 | C/A |
| rs554219 | 11 | G/C |
| rs614367 | 11 | A/G |
| rs75915166 | 11 | A/C |
| rs10771399 | 12 | A/C |
| rs12422552 | 12 | C/G |
| rs1292011 | 12 | A/G |
| rs17356907 | 12 | A/G |
| rs11571833 | 13 | A/T |
| rs2236007 | 14 | G/A |
| rs2588809 | 14 | A/G |
| rs941764 | 14 | G/A |
| rs999737 | 14 | G/A |
| rs11075995 | 16 | A/T |
| rs13329835 | 16 | G/A |
| rs17817449 | 16 | A/C |
| rs3803662 | 16 | A/G |
| rs6504950 | 17 | G/A |
| rs1436904 | 18 | A/C |
| rs527616 | 18 | C/G |
| rs3760982 | 19 | A/G |
| rs4808801 | 19 | A/G |
| rs8170 | 19 | A/G |
| rs2823093 | 21 | G/A |
| rs132390 | 22 | G/A |
| rs6001930 | 22 | G/A |

TABLE 16
Optimised list of Hispanic polymorphisms (n = 71).

| Polymorphism | Chromosome | Major allele/minor allele |
|---|---|---|
| rs11249433 | 1 | C/T |
| rs11552449 | 1 | T/C |
| rs4245739 | 1 | A/C |
| rs616488 | 1 | G/A |
| rs6678914 | 1 | A/G |
| rs12710696 | 2 | T/C |
| rs13387042 | 2 | A/G |
| rs1550623 | 2 | G/A |
| rs16857609 | 2 | T/C |
| rs2016394 | 2 | A/G |
| rs4849887 | 2 | T/C |
| rs12493607 | 3 | G/G |
| rs4973768 | 3 | T/C |
| rs6762644 | 3 | C/A |
| rs6828523 | 4 | A/C |
| rs7696175 | 4 | T/C |
| rs9790517 | 4 | T/C |
| rs10069690 | 5 | T/C |
| rs10472076 | 5 | C/T |
| rs10941679 | 5 | G/A |
| rs1353747 | 5 | G/T |
| rs1432679 | 5 | C/T |
| rs2067980 | 5 | G/A |
| rs889312 | 5 | C/A |
| rs11242675 | 6 | C/T |
| rs140068132 | 6 | G/A |
| rs204247 | 6 | G/A |
| rs2046210 | 6 | A/G |
| rs2180341 | 6 | G/A |
| rs17157903 | 7 | T/C |
| rs720475 | 7 | A/G |
| rs11780156 | 8 | T/C |
| rs13281615 | 8 | G/A |
| rs2943559 | 8 | G/A |
| rs6472903 | 8 | G/T |
| rs9693444 | 8 | A/C |
| rs1011970 | 9 | T/G |
| rs10759243 | 9 | A/C |
| rs865686 | 9 | T/G |
| rs10995190 | 10 | G/A |
| rs11199914 | 10 | T/C |
| rs11814448 | 10 | C/A |
| rs2380205 | 10 | C/T |
| rs2981579 | 10 | T/C |
| rs2981582 | 10 | T/C |
| rs704010 | 10 | T/C |
| rs7072776 | 10 | A/G |
| rs11820646 | 11 | T/C |
| rs3817198 | 11 | C/T |
| rs3903072 | 11 | T/G |
| rs10771399 | 12 | T/C |
| rs12422552 | 12 | C/G |
| rs1292011 | 12 | A/G |
| rs17356907 | 12 | G/A |
| rs2236007 | 14 | A/G |
| rs2588809 | 14 | T/C |
| rs941764 | 14 | G/A |
| rs999737 | 14 | C/T |
| rs11075995 | 16 | A/T |
| rs13329835 | 16 | G/A |
| rs17817449 | 16 | G/T |
| rs3803662 | 16 | T/C |
| rs6504950 | 17 | G/A |
| rs1436904 | 18 | G/T |
| rs527616 | 18 | C/G |
| rs2363956 | 19 | C/A |
| rs3760982 | 19 | A/G |
| rs4808801 | 19 | G/A |
| rs8170 | 19 | A/G |
| rs2823093 | 21 | G/A |
| rs6001930 | 22 | C/T |

TABLE 17
313 polymorphism panel.

| | | | | | | | Effect | β | OR | |
|---|---|---|---|---|---|---|---|---|---|---|
| Chro- | | GRCh37 | GRCh38 | | | Ref- | Allele | Overall | Overall | |
| mo- | | Position | Position | | Effect | erence | Freq- | Breast | Breast | |
| some | SNP | (hg19) | (hg38) | RS Number | Allele | Allele | uency | Cancer | Cancer | μ |
| 1 | 1_7917076_G_A | 7917076 | 7857016 | rs707475 | A | G | 0.3899 | −0.0409 | 0.9599 | 0.9690 |
| 1 | 1_10566215_A_G | 10566215 | 10506158 | rs616488 | G | A | 0.3290 | −0.0586 | 0.9431 | 0.9629 |
| 1 | 1_18807339_T_C | 18807339 | 18480845 | rs2992756 | C | T | 0.5145 | −0.0564 | 0.9452 | 0.9444 |
| 1 | 1_41380440_C_T | 41380440 | 40914768 | rs4233486 | T | C | 0.6438 | 0.0426 | 1.0435 | 1.0568 |
| 1 | 1_41389220_T_C | 41389220 | 40923548 | rs114282204 | C | T | 0.0169 | 0.1550 | 1.1677 | 1.0057 |
| 1 | 1_46670206_TC_T | 46670206 | 46204534 | 1: 46670206_ | T | TC | 0.2973 | 0.0447 | 1.0457 | 1.0274 |
| | | | | TC_T | | | | | | |
| 1 | 1_51467096_CT_C | 51467096 | 51001424 | 1: 51467096_ | C | CT | 0.4800 | 0.0374 | 1.0381 | 1.0369 |
| | | | | CT_C | | | | | | |
| 1 | 1_88156923_G_A | 88156923 | 87691240 | rs17426269 | A | G | 0.1487 | 0.0494 | 1.0506 | 1.0151 |
| 1 | 1_88428199_C_A | 88428199 | 87962516 | s2151842 | A | C | 0.2477 | −0.0387 | 0.9620 | 0.9813 |
| 1 | 1_100880328_A_T | 100880328 | 100414772 | rs612683 | T | A | 0.4097 | 0.0373 | 1.0380 | 1.0314 |
| 1 | 1_110198129_ | 110198129 | 109655507 | 1: 110198129_ | C | CAAA | 0.7755 | 0.0458 | 1.0469 | 1.0740 |
| | CAAAA_C | | | CAAA_C | | | | | | |
| 1 | 1_114445880_G_A | 114445880 | 113903258 | rs7513707 | A | G | 0.1664 | 0.0621 | 1.0641 | 1.0214 |
| 1 | 118141492_A_C | 118141492 | 117598870 | rs12406858 | C | A | 0.2657 | 0.0452 | 1.0462 | 1.0247 |
| 1 | 1_120257110_T_C | 120257110 | 119714487 | rs637868 | C | T | 0.5309 | 0.0385 | 1.0393 | 1.0421 |
| 1 | 1_121280613_A_G | 121280613 | 121538815 | rs11249433 | G | A | 0.4053 | 0.0881 | 1.0921 | 1.0761 |
| 1 | 1_121287994_A_G | 121287994 | 121546196 | rs111458676 | G | A | 0.1060 | −0.0673 | 0.9349 | 0.9862 |
| 1 | 1_145604302_C_CT | 145604302 | 145830811 | rs143384623 | CT | C | 0.3515 | −0.0399 | 0.9609 | 0.9727 |
| 1 | 1_149906413_T_C | 149906413 | 149934520 | rs11205303 | C | T | 0.4017 | 0.0548 | 1.0563 | 1.0458 |
| 1 | 1_155556971_G_A | 155556971 | 155587180 | rs12091730 | A | G | 0.2302 | 0.0499 | 1.0512 | 1.0237 |
| 1 | 1_168171052_CA_C | 168171052 | 168201814 | rs761575824 | C | CA | 0.1097 | −0.0680 | 0.9343 | 0.9856 |
| 1 | 1_172328767_T_TA | 172328767 | 172359627 | rs11463354 | TA | T | 0.3305 | −0.0435 | 0.9574 | 0.9721 |
| 1 | 1_201437832_C_T | 201437832 | 201468704 | rs35383942 | T | C | 0.0559 | 0.0917 | 1.0960 | 1.0108 |
| 1 | 1_202184600_C_T | 202184600 | 202215472 | rs6686987 | T | C | 0.3992 | −0.0065 | 0.9935 | 0.9948 |
| 1 | 1_203770448_T_A | 203770448 | 203801320 | rs7514172 | A | T | 0.2715 | 0.0498 | 1.0511 | 1.0279 |
| 1 | 1_204502514_T_ | 204502514 | 204533386 | rs11268668 | TTCTG | T | 0.8028 | −0.0321 | 0.9684 | 0.9499 |
| | TTCTGAAACAGGG | | | | AAACA | | | | | |
| | | | | | GGG | | | | | |
| 1 | 1_208076291_G_A | 208076291 | 207902946 | rs2785646 | A | G | 0.3337 | −0.0366 | 0.9641 | 0.9762 |
| 1 | 1_217053815_T_G | 217053815 | 216880473 | rs2576261 | G | T | 0.3280 | 0.0417 | 1.0426 | 1.0281 |
| 1 | 1_217220574_G_A | 217220574 | 217047232 | rs11117758 | A | G | 0.2107 | −0.0440 | 0.9570 | 0.9819 |
| 1 | 1_220671050_C_T | 220671050 | 220497708 | rs11118563 | T | C | 0.2415 | 0.0418 | 1.0427 | 1.0207 |
| 1 | 1_242034263_A_G | 242034263 | 241870961 | rs72755295 | G | A | 0.0305 | 0.1428 | 1.1535 | 1.0094 |
| 2 | 2_10138983_T_C | 10138983 | 9998855 | rs78425380 | C | T | 0.1160 | 0.0603 | 1.0622 | 1.0145 |
| 2 | 2_19315675_T_A | 19315675 | 19115914 | rs6743383 | A | T | 0.5599 | 0.0331 | 0.9674 | 0.9639 |
| 2 | 2_25129473_A_G | 25129473 | 24906604 | rs6725517 | G | A | 0.4082 | −0.0427 | 0.9582 | 0.9662 |
| 2 | 2_29179452_G_C | 29179452 | 28956586 | rs12472404 | C | G | 0.2287 | −0.0066 | 0.9934 | 0.9970 |
| 2 | 2_29615233_T_C | 29615233 | 29392367 | rs4322799 | C | T | 0.2622 | −0.0427 | 0.9582 | 0.9782 |
| 2 | 2_39699510_C_CT | 39699510 | 39472369 | rs553796823 | CT | C | 0.4659 | −0.0402 | 0.9606 | 0.9636 |
| 2 | 2_70172587_G_A | 70172587 | 69945455 | rs6756513 | A | G | 0.2787 | −0.0412 | 0.9596 | 0.9776 |
| 2 | 2_88358825_G_C | 88358825 | 88059306 | rs1036759 | C | G | 0.3081 | 0.0473 | 1.0484 | 1.0301 |
| 2 | 2_121058254_A_G | 121058254 | 120300678 | rs6746250 | G | A | 0.7047 | −0.0334 | 0.9672 | 0.9542 |
| 2 | 2_121089731_T_C | 121089731 | 120332155 | rs17625845 | C | T | 0.1943 | 0.0427 | 0.9582 | 0.9838 |
| 2 | 2_121159205_G_A | 121159205 | 120401629 | rs10164550 | A | G | 0.3527 | −0.0440 | 0.9570 | 0.9699 |
| 2 | 2_121246568_T_C | 121246568 | 120488992 | rs10179592 | C | T | 0.8970 | 0.0992 | 1.1043 | 1.1958 |
| 2 | 2_172974566_C_G | 172974566 | 172109838 | rs17726078 | G | C | 0.4743 | −0.0473 | 0.9538 | 0.9567 |
| 2 | 2_174212910_A_G | 174212910 | 173348182 | rs1550622 | G | A | 0.8450 | 0.0593 | 1.0611 | 1.1059 |
| 2 | 2_192381934_C_T | 192381934 | 191517208 | rs2356656 | T | C | 0.8588 | 0.0316 | 1.0321 | 1.0559 |
| 2 | 2_202204741_T_C | 202204741 | 201340018 | rs10197246 | C | T | 0.7210 | −0.0492 | 0.9520 | 0.9320 |
| 2 | 2_217920769_G_T | 217920769 | 217056046 | rs4442975 | T | G | 0.5001 | −0.1318 | 0.8765 | 0.8803 |
| 2 | 2_217955896_GA_G | 217955896 | 217091173 | 2: 217955896_ | G | GA | 0.0382 | −0.2016 | 0.8174 | 0.9861 |
| | | | | GA_G | | | | | | |
| 2 | 2_218292158_C_G | 218292158 | 217427435 | rs11693806 | G | C | 0.7309 | −0.0757 | 0.9271 | 0.8963 |
| 2 | 2_218714845_G_A | 218714845 | 217850122 | rs3791977 | A | G | 0.3917 | −0.0431 | 0.9578 | 0.9672 |
| 2 | 2_241388857_C_A | 241388857 | 240449440 | rs4676356 | A | C | 0.9772 | −0.1232 | 0.8841 | 0.7863 |
| 3 | 3_4742251_A_G | 4742251 | 4700567 | rs6762558 | G | A | 0.3802 | 0.0616 | 1.0635 | 1.0489 |
| 3 | 3_27353716_C_A | 27353716 | 27312225 | s552647 | A | C | 0.5259 | 0.0748 | 1.0777 | 1.0834 |
| 3 | 3_27388664_C_G | 27388664 | 27347173 | rs62255657 | G | C | 0.2735 | 0.0502 | 1.0515 | 1.0284 |
| 3 | 3_29294845_C_T | 29294845 | 29253354 | rs112476261 | T | C | 0.0163 | −0.1281 | 0.8798 | 0.9961 |
| 3 | 3_30684907_C_T | 30684907 | 30643415 | rs17838698 | T | C | 0.2975 | 0.0592 | 1.0610 | 1.0366 |
| 3 | 3_46888198_T_C | 46888198 | 46846708 | rs56387622 | C | T | 0.1032 | −0.0806 | 0.9226 | 0.9841 |
| 3 | 3_49709912_C_CT | 49709912 | 49672479 | rs371314787 | CT | C | 0.2873 | −0.0367 | 0.9640 | 0.9794 |
| 3 | 3_55970777_A_AT | 55970777 | 55936749 | rs138866686 | AT | A | 0.0305 | −0.1195 | 0.8874 | 0.9932 |
| 3 | 3_59373745_C_T | 59373745 | 59388019 | rs2886671 | T | C | 0.4294 | −0.0394 | 0.9614 | 0.9671 |
| 3 | 3_63887449_T_TTG | 63887449 | 63901773 | | TTG | T | 0.1297 | 0.0648 | 1.0669 | 1.0174 |
| 3 | 3_71620370_T_G | 71620370 | 71571219 | rs9825432 | G | T | 0.6382 | −0.0374 | 0.9633 | 0.9537 |
| 3 | 3_87037543_A_G | 87037543 | 86988393 | rs13066793 | G | A | 0.0921 | −0.0723 | 0.9303 | 0.9872 |
| 3 | 3_99403877_G_A | 99403877 | 99685033 | rs639355 | A | G | 0.4852 | −0.0376 | 0.9631 | 0.9645 |
| 3 | 3_141112859_ | 141112859 | 141394017 | 3: 141112859_ | C | CTT | 0.4149 | 0.0551 | 1.0566 | 1.0476 |
| | CTT_C | | | CTT_C | | | | | | |
| 3 | 3_172285237_G_A | 172285237 | 172567447 | rs58058861 | A | G | 0.2131 | 0.0422 | 1.0431 | 1.0185 |
| 3 | 3_189774456_C_T | 189774456 | 190056667 | rs9882792 | T | C | 0.2235 | −0.0478 | 0.9533 | 0.9792 |
| 4 | 4_38784633_G_T | 38784633 | 38783012 | rs10012017 | T | G | 0.2493 | 0.0489 | 1.0501 | 1.0251 |
| 4 | 4_84370124_TAA_T | 84370124 | 83448971 | rs774021038 | TA | TAA | 0.5324 | −0.0464 | 0.9547 | 0.9523 |
| | A | | | | | | | | | |
| 4 | 4_89240476_G_A | 89240476 | 88319324 | rs17014016 | A | G | 0.4395 | 0.0352 | 1.0358 | 1.0317 |
| 4 | 4_92594859_TTCTT | 92594859 | 91673708 | rs775780062 | T | TTCTTT | 0.4445 | −0.0407 | 0.9601 | 0.9649 |
| | TC_T | | | | | C | | | | |
| 4 | 4_106069013_G_T | 106069013 | 105147856 | rs62331150 | T | G | 0.2289 | 0.0471 | 1.0482 | 1.0222 |
| 4 | 4_126752992_A_AA | 126752992 | 125831837 | | AAT | A | 0.5167 | −0.0377 | 0.9630 | 0.9621 |
| | T | | | | | | | | | |
| 4 | 4_143467195_C_T | 143467195 | 142546042 | rs56039025 | T | C | 0.1115 | −0.0569 | 0.9447 | 0.9877 |
| 4 | 4_151218296_CATA | 151218296 | 150297144 | rs745707748 | C | CATATT | 0.6533 | 0.0388 | 1.0396 | 1.0524 |
| | TTT_C | | | | | T | | | | |
| 4 | 4_175842495_G_A | 175842495 | 174921344 | rs28436676 | A | G | 0.1161 | −0.0898 | 0.9141 | 0.9802 |
| 4 | 4_175847436_C_A | 175847436 | 174926285 | rs62334414 | A | C | 0.3433 | 0.0348 | 1.0354 | 1.0245 |
| 4 | 4_187503758_A_T | 187503758 | 186582604 | | T | A | 0.4471 | 0.0357 | 1.0363 | 1.0328 |
| 5 | 5_345109_T_C | 345109 | 344994 | rs62641919 | C | T | 0.0544 | 0.0840 | 1.0876 | 1.0096 |
| 5 | 5_1279790_C_T | 1279790 | 1279675 | rs10069690 | T | C | 0.2592 | 0.0617 | 1.0636 | 1.0333 |
| 5 | 5_1296255_A_AG | 1296255 | 1296140 | rs3215401 | AG | A | 0.3072 | −0.0549 | 0.9466 | 0.9674 |
| 5 | 5_1353077_T_C | 1353077 | 1352962 | rs62329727 | C | T | 0.0121 | 0.1552 | 1.1679 | 1.0041 |
| 5 | 5_2777029_G_A | 2777029 | 2776915 | rs4866496 | A | G | 0.4139 | 0.0391 | 1.0399 | 1.0333 |
| 5 | 5_16231194_G_C | 16231194 | 16231085 | rs17611291 | C | G | 0.5594 | −0.0426 | 0.9583 | 0.9539 |
| 5 | 5_32579616_TCA_T | 32579616 | 32579510 | rs770436441 | T | TCA | 0.4844 | 0.0363 | 1.0370 | 1.0361 |
| 5 | 5_44508264_G_GT | 44508264 | 44508162 | rs138335056 | GT | G | 0.1265 | −0.1177 | 0.8890 | 0.9721 |
| 5 | 5_44619502_A_G | 44619502 | 44619400 | rs187108781 | G | A | 0.1549 | −0.1101 | 0.8957 | 0.9680 |
| 5 | 5_44649944_C_T | 44649944 | 44649842 | rs4613718 | T | C | 0.6010 | 0.0492 | 1.0504 | 1.0615 |
| 5 | 5_44706498_A_G | 44706498 | 44706396 | rs10941679 | G | A | 0.2481 | 0.0497 | 1.0510 | 1.0254 |
| 5 | 5_44853593_G_C | 44853593 | 44853491 | rs17343002 | C | G | 0.3081 | −0.0336 | 0.9670 | 0.9797 |
| 5 | 5_52679539_C_CA | 52679539 | 53383709 | | CA | C | 0.0998 | 0.0571 | 1.0588 | 1.0118 |
| 5 | 5_55662540_C_CT | 55662540 | 56366713 | rs553874618 | CT | C | 0.3631 | −0.0458 | 0.9552 | 0.9678 |
| 5 | 5_55965167_C_T | 55965167 | 56669340 | rs889310 | T | C | 0.5576 | 0.0394 | 1.0402 | 1.0453 |
| 5 | 5_56023083_T_G | 56023083 | 56727256 | rs16886165 | G | T | 0.1583 | 0.1366 | 1.1464 | 1.0469 |
| 5 | 5_56042972_C_T | 56042972 | 56747145 | rs76250845 | T | C | 0.0521 | 0.0865 | 1.0904 | 1.0094 |
| 5 | 5_56045081_T_C | 56045081 | 56749254 | rs11949391 | C | T | 0.1655 | −0.0564 | 0.9452 | 0.9819 |
| 5 | 5_58241712_C_T | 58241712 | 58945885 | rs113778879 | T | C | 0.5750 | −0.0434 | 0.9575 | 0.9518 |
| 5 | 5_71965007_G_A | 71965007 | 72669180 | rs3010266 | A | G | 0.2572 | −0.0410 | 0.9598 | 0.9794 |
| 5 | 5_73234583_T_C | 73234583 | 73938758 | rs157557 | C | T | 0.3213 | −0.0363 | 0.9644 | 0.9772 |
| 5 | 5_77155397_GT_G | 77155397 | 77859573 | rs767431357 | G | GT | 0.3466 | −0.0408 | 0.9600 | 0.9725 |
| 5 | 5_79180995_G_GA | 79180995 | 79885172 | rs34525310 | GA | G | 0.1755 | 0.0328 | 1.0333 | 1.0117 |
| 5 | 5_81512947_TA_T | 81512947 | 82217128 | 5: 81512947_ | T | TA | 0.2503 | −0.0598 | 0.9420 | 0.9712 |
| | | | | TA_T | | | | | | |
| 5 | 5_90789470_G_A | 90789470 | 91493653 | rs332529 | A | G | 0.1580 | −0.0564 | 0.9452 | 0.9827 |
| 5 | 5_104300273_G_T | 104300273 | 104964572 | rs17157372 | T | G | 0.1810 | −0.0487 | 0.9525 | 0.9829 |
| 5 | 5_122478676_C_A | 122478676 | 123142982 | rs335160 | A | C | 0.7448 | −0.0386 | 0.9621 | 0.9444 |
| 5 | 5_122705244_C_T | 122705244 | 123369550 | rs1428387 | T | C | 0.0306 | 0.0944 | 1.0990 | 1.0061 |
| 5 | 5_131640536_A_G | 131640536 | 132304843 | rs6860806 | G | A | 0.5427 | 0.0392 | 1.0400 | 1.0439 |
| 5 | 5_132407058_C_T | 132407058 | 133071366 | rs6596100 | T | C | 0.2450 | −0.0388 | 0.9619 | 0.9814 |
| 5 | 5_158244083_C_T | 158244083 | 158817075 | rs1432679 | T | C | 0.5683 | −0.0677 | 0.9345 | 0.9270 |
| 5 | 5_169591460_T_C | 169591460 | 170164456 | rs10074269 | C | T | 0.3345 | 0.0412 | 1.0421 | 1.0283 |
| 5 | 5_173358154_G_A | 173358154 | 173931151 | rs6864691 | A | G | 0.4074 | 0.0365 | 1.0372 | 1.0305 |
| 5 | 5_176134882_T_C | 176134882 | 176707881 | rs4868701 | C | T | 0.5422 | 0.0363 | 1.0370 | 1.0405 |
| 6 | 6_13713366_G_C | 13713366 | 13713134 | rs418053 | C | G | 0.5691 | 0.0553 | 0.9462 | 0.9397 |
| 6 | 6_16399557_C_T | 16399557 | 16399326 | rs3819405 | T | C | 0.3299 | −0.0373 | 0.9634 | 0.9760 |
| 6 | 6_18783140_G_A | 18783140 | 18782909 | rs12211970 | A | G | 0.6200 | 0.0326 | 1.0331 | 1.0415 |
| 6 | 6_20537845_CA_C | 20537845 | 20537614 | rs769485514 | C | CA | 0.4733 | −0.0391 | 0.9617 | 0.9640 |
| 6 | 6_21923810_T_C | 21923810 | 21923579 | rs9358466 | C | T | 0.4303 | −0.0321 | 0.9684 | 0.9730 |
| 6 | 6_27425644_G_C | 27425644 | 27457865 | rs34196306 | C | G | 0.0815 | −0.0737 | 0.9290 | 0.9885 |
| 6 | 6_43227141_G_A | 43227141 | 43259403 | rs111342015 | A | G | 0.0985 | −0.0640 | 0.9380 | 0.9878 |
| 6 | 6_82263549_AAT_A | 82263549 | 81553832 | rs10623112 | A | AAT | 0.4262 | 0.0477 | 1.0489 | 1.0421 |
| 6 | 6_85912194_CAA_C | 85912194 | 85202476 | 6: 85912194_ | C | CAA | 0.0604 | 0.0762 | 1.0792 | 1.0096 |
| | | | | CAA_C | | | | | | |
| 6 | 6_87803819_T_C | 87803819 | 87094101 | rs73754909 | C | T | 0.2770 | 0.0383 | 1.0390 | 1.0217 |
| 6 | 6_130341728_C_CT | 130341728 | 130020583 | rs55941023 | CT | C | 0.7116 | 0.0472 | 1.0483 | 1.0700 |
| 6 | 6_149595505_T_C | 149595504 | 149274369 | rs2121348 | C | T | 0.2061 | −0.0476 | 0.9535 | 0.9809 |
| 6 | 6_151949806_A_C | 151949806 | 151628671 | rs6913578 | C | A | 0.3083 | 0.0703 | 1.0728 | 1.0454 |
| 6 | 6_151955914_A_G | 151955914 | 151634779 | rs60954078 | G | A | 0.0713 | 0.1449 | 1.1559 | 1.0224 |
| 6 | 6_152022664_CAAA | 152022664 | 151701529 | 6: 152022664_ | C | CAAAAA | 0.6119 | 0.0137 | 1.0138 | 1.0170 |
| | AAAA_C | | | CAAAAAAA_C | | AA | | | | |
| 6 | 6_152023191_G_A | 152023191 | 151702056 | rs851984 | A | G | 0.3965 | 0.0626 | 1.0646 | 1.0519 |
| 6 | 6_152055978_A_T | 152055978 | 151734843 | rs6904031 | T | A | 0.0627 | 0.0740 | 1.0768 | 1.0096 |
| 6 | 6_152432902_C_T | 152432902 | 152111767 | rs910416 | T | C | 0.5146 | 0.0649 | 1.0671 | 1.0702 |
| 6 | 6_169006947_C_G | 169006947 | 168606267 | rs9364472 | G | C | 0.5202 | −0.0308 | 0.9697 | 0.9687 |
| 6 | 6_170332621_T_C | 170332621 | 170017397 | rs6940159 | C | T | 0.6158 | 0.0373 | 1.0380 | 1.0474 |
| 7 | 7_21940960_A_G | 21940960 | 21901342 | rs7971 | G | A | 0.3515 | −0.0467 | 0.9544 | 0.9682 |
| 7 | 7_25569548_C_T | 25569548 | 25529928 | rs289997 | T | C | 0.1667 | −0.0486 | 0.9526 | 0.9842 |
| 7 | 7_28869017_G_A | 28869017 | 28829400 | rs74765302 | A | G | 0.1072 | −0.0572 | 0.9444 | 0.9881 |
| 7 | 7_55192256_A_C | 55192256 | 55124563 | rs13244925 | C | A | 0.5497 | −0.0349 | 0.9657 | 0.9626 |
| 7 | 7_91459189_A_ATT | 91459189 | 91829875 | | ATT | A | 0.3286 | 0.0452 | 1.0462 | 1.0306 |
| 7 | 7_94113799_T_C | 94113799 | 94484487 | rs17268829 | C | T | 0.2792 | 0.0449 | 1.0459 | 1.0258 |
| 7 | 7_98005235_G_A | 98005235 | 98375923 | rs4439053 | A | G | 0.1627 | 0.0467 | 0.9544 | 0.9852 |
| 7 | 7_99948655_T_G | 99948655 | 100351032 | rs111963714 | G | T | 0.2109 | 0.0420 | 1.0429 | 1.0182 |
| 7 | 7_101552440_G_A | 101552440 | 101909160 | rs71559437 | A | G | 0.1255 | −0.0568 | 0.9448 | 0.9862 |
| 7 | 7_102481842_T_C | 102481842 | 102841395 | rs7800548 | C | T | 0.3416 | 0.0418 | 1.0427 | 1.0294 |
| 7 | 7_130656911_C_T | 130656911 | 130972152 | rs12706954 | T | C | 0.3734 | −0.0476 | 0.9535 | 0.9656 |
| 7 | 7_130674481_G_A | 130674481 | 130989722 | rs68056147 | A | G | 0.2971 | 0.0416 | 1.0425 | 1.0254 |
| 7 | 7_139943702_CT_C | 139943702 | 140243902 | rs201664599 | C | CT | 0.5381 | 0.0582 | 1.0599 | 1.0655 |
| 7 | 7_144048902_G_T | 144048902 | 144351809 | rs62485509 | T | G | 0.2284 | −0.0563 | 0.9453 | 0.9751 |
| 8 | 8_170692_T_C | 170692 | 220692 | rs66823261 | C | T | 0.2227 | 0.0477 | 1.0489 | 1.0219 |
| 8 | 8_17787610_CT_C | 17787610 | 17930101 | 8: 17787610_ | C | CT | 0.6230 | −0.0377 | 0.9630 | 0.9544 |
| | | | | CT_C | | | | | | |
| 8 | 8_23447496_A_G | 23447496 | 23589983 | rs1028016 | G | A | 0.6487 | −0.0389 | 0.9618 | 0.9511 |
| 8 | 8_23663653_C_A | 23663653 | 23806140 | rs310295 | A | C | 0.4032 | 0.0335 | 1.0341 | 1.0277 |
| 8 | 8_29509616_A_C | 29509616 | 29652100 | rs9693444 | C | A | 0.6756 | −0.0601 | 0.9417 | 0.9227 |
| 8 | 8_36858483_A_G | 36858483 | 37000965 | rs13365225 | G | A | 0.1820 | −0.0760 | 0.9268 | 0.9735 |
| 8 | 8_76230943_A_G | 76230943 | 75318708 | rs1511243 | G | A | 0.8282 | 0.0755 | 1.0784 | 1.1341 |
| 8 | 8_76333056_C_T | 76333056 | 75420821 | rs72658084 | T | C | 0.0878 | 0.1129 | 1.1195 | 1.0211 |
| 8 | 8_76378165_G_T | 76378165 | 75465930 | rs1533366 | T | G | 0.3595 | 0.0391 | 0.9617 | 0.9726 |
| 8 | 8_102483100_T_C | 102483100 | 101470872 | rs62517052 | C | T | 0.0967 | 0.0593 | 1.0611 | 1.0118 |
| 8 | 8_106358620_A_T | 106358620 | 105346392 | rs12546444 | T | A | 0.1003 | −0.0745 | 0.9282 | 0.9856 |
| 8 | 8_117209548_A_G | 117209548 | 116197325 | rs13267382 | G | A | 0.6445 | −0.0417 | 0.9592 | 0.9480 |
| 8 | 8_120862186_A_G | 120862186 | 119849946 | rs62526620 | G | A | 0.1318 | 0.0527 | 1.0541 | 1.0143 |
| 8 | 8_124563705_T_C | 124563705 | 123551465 | rs35542655 | C | T | 0.1458 | 0.0477 | 1.0489 | 1.0143 |
| 8 | 8_124571581_G_A | 124571581 | 123559341 | rs12541094 | A | G | 0.4173 | 0.0340 | 1.0346 | 1.0291 |
| 8 | 8_124739913_T_G | 124739913 | 123727673 | rs7842619 | G | T | 0.3985 | 0.0466 | 1.0477 | 1.0384 |
| 8 | 8_128213561_C_CA | 128213561 | 127201316 | rs35961416 | CA | C | 0.4153 | −0.0430 | 0.9579 | 0.9653 |
| 8 | 8_128370949_C_G | 128370949 | 127358703 | rs12550713 | G | C | 0.4020 | 0.0642 | 1.0663 | 1.0540 |
| 8 | 8_128372172_A_G | 128372172 | 127359926 | rs10096351 | G | A | 0.5446 | 0.0597 | 1.0615 | 1.0681 |
| 8 | 8_129199566_G_A | 129199566 | 128187320 | rs1016578 | A | G | 0.1717 | 0.0615 | 1.0634 | 1.0219 |
| 8 | 8_143669254_A_G | 143669254 | 142587893 | rs7830152 | G | A | 0.3390 | −0.0346 | 0.9660 | 0.9771 |
| 9 | 9_6880263_A_G | 6880263 | 6880263 | rs10975870 | G | A | 0.2855 | 0.0348 | 1.0354 | 1.0203 |
| 9 | 9_21964882_CAAAA | 21964882 | 21964883 | rs745322029 | C | CAAAA | 0.3184 | 0.0550 | 1.0565 | 1.0363 |
| | C | | | | | | | | | |
| 9 | 9_22041998_C_G | 22041998 | 22041999 | rs17694493 | G | C | 0.1393 | 0.0289 | 1.0293 | 1.0082 |
| 9 | 9_36928288_T_C | 36928288 | 36928291 | rs4880038 | C | T | 0.5349 | 0.0249 | 1.0252 | 1.0272 |
| 9 | 9_87782211_T_C | 87782211 | 85167296 | rs665889 | C | T | 0.5094 | 0.0361 | 1.0368 | 1.0378 |
| 9 | 9_98362587_T_C | 98362587 | 95600305 | rs10120432 | C | T | 0.0940 | 0.0576 | 1.0593 | 1.0112 |
| 9 | 9_110303808_TAA | 110303808 | 107541527 | 9: 110303808_ | T | TAA | 0.2065 | 0.0797 | 1.0830 | 1.0346 |
| | T | | | TAAT | | | | | | |
| 9 | 9_110837073_A_G | 110837073 | 108074792 | rs10816625 | G | A | 0.0630 | 0.1158 | 1.1228 | 1.0155 |
| 9 | 9_110837176_C_T | 110837176 | 108074895 | rs13294895 | T | C | 0.1750 | 0.0653 | 1.0675 | 1.0238 |
| 9 | 9_110849525_G_T | 110849525 | 108087244 | rs7848334 | T | G | 0.5977 | 0.0153 | 1.0154 | 1.0185 |
| 9 | 9_110885479_C_T | 110885479 | 108123199 | rs630965 | T | C | 0.6222 | 0.0877 | 1.0917 | 1.1173 |
| 9 | 9_119313486_A_G | 119313486 | 116551207 | rs1895062 | G | A | 0.4087 | −0.0462 | 0.9549 | 0.9634 |
| 9 | 9_129424719_A_G | 129424719 | 126662440 | rs3861871 | G | A | 0.4577 | 0.0382 | 0.9625 | 0.9660 |
| 9 | 9_136146597_C_T | 136146597 | 133271182 | rs550057 | T | C | 0.2727 | 0.0400 | 1.0408 | 1.0224 |
| 10 | 10_5794652_A_G | 5794652 | 5752689 | rs55910451 | G | A | 0.2137 | 0.0470 | 1.0481 | 1.0207 |
| 10 | 10_13892298_G_A | 13892298 | 13850298 | rs10796139 | A | G | 0.4376 | 0.0371 | 1.0378 | 1.0334 |
| 10 | 10_22032942_A_G | 22032942 | 21744013 | rs7072776 | G | A | 0.7085 | −0.0580 | 0.9436 | 0.9217 |
| 10 | 10_22477776_ | 22477776 | 22188847 | rs762131501 | A | ACC | 0.0202 | 0.1687 | 1.1838 | 1.0074 |
| | ACC_A | | | | | | | | | |
| 10 | 10_22861490_A_C | 22861490 | 22572561 | rs10764337 | C | A | 0.9370 | 0.0875 | 1.0914 | 1.1787 |
| 10 | 10_38523626_C_A | 38523626 | 38234698 | rs2384736 | A | C | 0.3698 | 0.0404 | 1.0412 | 1.0307 |
| 10 | 10_64299890_A_G | 64299890 | 62540131 | rs10995201 | G | A | 0.1603 | −0.1345 | 0.8742 | 0.9601 |
| 10 | 10_64819996_G_T | 64819996 | 63060236 | rs6479868 | T | G | 0.1958 | 0.0472 | 1.0483 | 1.0190 |
| 10 | 10_71335574_C_T | 71335574 | 69575818 | rs111833376 | T | C | 0.3179 | −0.0404 | 0.9604 | 0.9750 |
| 10 | 10_80851257_G_T | 80851257 | 79091500 | rs719338 | T | G | 0.6172 | −0.0805 | 0.9227 | 0.9068 |
| 10 | 10_80886726_A_G | 80886726 | 79126969 | rs4980029 | G | A | 0.1631 | 0.0762 | 1.0792 | 1.0260 |
| 10 | 10_95292187_ | 95292187 | 93532430 | 10: 95292187_ | C | CAA | 0.8234 | −0.0512 | 0.9501 | 0.9195 |
| | CAA_C | | | CAAC | | | | | | |
| 10 | 10_114777670_C_T | 114777670 | 113017911 | rs10885405 | T | C | 0.4631 | 0.0472 | 1.0483 | 1.0453 |
| 10 | 10_115128491_T_C | 115128491 | 113368732 | rs12250948 | C | T | 0.7846 | −0.0592 | 0.9425 | 0.9118 |
| 10 | 10_123095209_G_A | 123095209 | 121335695 | rs9421410 | A | G | 0.3269 | 0.0538 | 0.9476 | 0.9660 |
| 10 | 10_123340107_A_G | 123340107 | 121580593 | rs45631580 | G | A | 0.0656 | 0.1508 | 1.1628 | 1.0215 |
| 10 | 10_123340431_GC_ | 123340431 | 121580917 | 10: 123340431_ | G | GC | 0.5963 | −0.2408 | 0.7860 | 0.7611 |
| | G | | | GC_G | | | | | | |
| 10 | 10_123349324_A_T | 123349324 | 121589810 | rs45631563 | T | A | 0.0484 | −0.2609 | 0.7704 | 0.9779 |
| 11 | 11_433617_T_C | 433617 | 433617 | rs7394715 | C | T | 0.7969 | −0.0437 | 0.9572 | 0.9330 |
| 11 | 11_803017_A_G | 803017 | 803017 | rs6597981 | G | A | 0.5167 | 0.0457 | 1.0468 | 1.0489 |
| 11 | 11_1895708_C_A | 1895708 | 1874478 | rs4980386 | A | C | 0.3924 | −0.0762 | 0.9266 | 0.9432 |
| 11 | 11_18664241_T_G | 18664241 | 18642694 | rs10832963 | G | T | 0.7293 | 0.0461 | 1.0472 | 1.0700 |
| 11 | 11_42844441_C_T | 42844441 | 42822891 | rs4472923 | T | C | 0.3279 | −0.0336 | 0.9670 | 0.9784 |
| 11 | 11_44368892_G_A | 44368892 | 44347342 | rs10838267 | A | G | 0.5495 | 0.0374 | 1.0381 | 1.0423 |
| 11 | 11_46318032_C_G | 46318032 | 46296481 | rs77047825 | G | C | 0.0659 | −0.0748 | 0.9279 | 0.9905 |
| 11 | 11_65553492_C_A | 65553492 | 65786021 | rs12287832 | A | C | 0.1867 | 0.0425 | 1.0434 | 1.0163 |
| 11 | 11_65572431_G_A | 65572431 | 65804960 | rs10896047 | A | G | 0.4886 | −0.0347 | 0.9659 | 0.9670 |
| 11 | 11_69328130_A_T | 69328130 | 69513362 | rs35039974 | T | A | 0.2130 | −0.0423 | 0.9586 | 0.9824 |
| 11 | 11_69330983_G_A | 69330983 | 69516215 | rs661204 | A | G | 0.1250 | 0.1022 | 1.1076 | 1.0271 |
| 11 | 11_69331418_C_T | 69331418 | 69516650 | rs78540526 | T | C | 0.0753 | 0.1782 | 1.1951 | 1.0296 |
| 11 | 11_103614438_T_G | 103614438 | 103743710 | rs7125780 | G | T | 0.6572 | 0.0147 | 1.0148 | 1.0196 |
| 11 | 11_108267402_C_CA | 108267402 | 108396675 | rs199504893 | CA | C | 0.4173 | −0.0022 | 0.9978 | 0.9982 |
| 11 | 11_111696440_T_C | 111696440 | 111825716 | rs610437 | C | T | 0.6221 | −0.0396 | 0.9612 | 0.9523 |
| 11 | 11_116727936_A_T | 116727936 | 116857220 | rs625145 | T | A | 0.2046 | −0.0423 | 0.9586 | 0.9831 |
| 11 | 11_122966626_A_G | 122966626 | 123095918 | rs7121616 | G | A | 0.2922 | −0.0383 | 0.9624 | 0.9782 |
| 11 | 11_129243417_T_G | 129243417 | 129373522 | rs7939702 | G | T | 0.8620 | −0.0543 | 0.9471 | 0.9110 |
| 11 | 11_129461016_A_G | 129461016 | 129591121 | rs11822830 | G | A | 0.6016 | 0.0453 | 1.0463 | 1.0565 |
| 12 | 12_293626_A_G | 293626 | 184460 | rs797736 | G | A | 0.3711 | 0.0401 | 1.0409 | 1.0306 |
| 12 | 12_14413931_G_C | 14413931 | 14260997 | rs12422552 | C | G | 0.2619 | 0.0484 | 1.0496 | 1.0261 |
| 12 | 12_28149568_C_T | 28149568 | 27996635 | rs788458 | T | C | 0.1170 | −0.0620 | 0.9399 | 0.9860 |
| 12 | 12_28174817_C_T | 28174817 | 28021884 | rs7297051 | T | C | 0.2421 | −0.0856 | 0.9180 | 0.9607 |
| 12 | 12_28347382_C_T | 28347382 | 28194449 | rs11049431 | T | C | 0.2153 | −0.0521 | 0.9492 | 0.9783 |
| 12 | 12_29140260_G_A | 29140260 | 28987327 | rs1027113 | A | G | 0.9126 | 0.0647 | 1.0668 | 1.1257 |
| 12 | 12_57146069_T_G | 57146069 | 56752285 | rs2277339 | G | T | 0.1037 | −0.0579 | 0.9437 | 0.9884 |
| 12 | 12_70798355_A_T | 70798355 | 70404575 | rs2870876 | T | A | 0.1810 | 0.0469 | 1.0480 | 1.0175 |
| 12 | 12_83064195_G_GA | 83064195 | 82670416 | rs111622698 | GA | G | 0.0992 | 0.0671 | 1.0694 | 1.0138 |
| 12 | 12_85004551_C_T | 85004551 | 84610772 | rs10862899 | T | C | 0.4955 | 0.0348 | 1.0354 | 1.0354 |
| 12 | 12_96027759_A_G | 96027759 | 95633983 | rs17356907 | G | A | 0.2963 | −0.0867 | 0.9170 | 0.9514 |
| 12 | 12_103097887_C_T | 103097887 | 102704109 | rs7132703 | T | C | 0.1175 | 0.0546 | 1.0561 | 1.0132 |
| 12 | 12_111600134_G_T | 111600134 | 111162330 | rs11065822 | T | G | 0.3715 | −0.0442 | 0.9568 | 0.9681 |
| 12 | 12_115108136_T_C | 115108136 | 114670331 | rs1061657 | C | T | 0.2615 | 0.0465 | 1.0476 | 1.0251 |
| 12 | 12_115796577_A_G | 115796577 | 115358772 | rs11067551 | G | A | 0.1959 | −0.0428 | 0.9581 | 0.9837 |
| 12 | 12_115835836_T_C | 115835836 | 115398031 | rs2454399 | C | T | 0.4171 | −0.0813 | 0.9219 | 0.9359 |
| 12 | 12_120832146_C_T | 120832146 | 120394343 | rs206966 | T | C | 0.1593 | 0.0516 | 1.0530 | 1.0169 |
| 13 | 13_32839990_G_A | 32839990 | 32265853 | rs56404467 | A | G | 0.0174 | 0.0424 | 1.0433 | 1.0015 |
| 13 | 13_32972626_A_T | 32972626 | 32398489 | rs11571833 | T | A | 0.0079 | 0.2687 | 1.3083 | 1.0049 |
| 13 | 13_43501356_A_G | 43501356 | 42927220 | rs9315973 | G | A | 0.8303 | 0.0517 | 1.0531 | 1.0901 |
| 13 | 13_73806982_T_C | 73806982 | 73232845 | rs12870942 | C | T | 0.3153 | 0.0345 | 1.0351 | 1.0223 |
| 13 | 13_73960952_A_G | 73960952 | 73386815 | rs2181965 | G | A | 0.7618 | 0.0399 | 1.0407 | 1.0630 |
| 14 | 14_37128564_C_A | 37128564 | 36659359 | rs34914085 | A | C | 0.2122 | −0.0733 | 0.9293 | 0.9702 |
| 14 | 14_37228504_C_T | 37228504 | 36759299 | rs2253012 | T | C | 0.4434 | 0.0390 | 1.0398 | 1.0356 |
| 14 | 14_68660428_T_C | 68660428 | 68193711 | rs2588809 | C | T | 0.8345 | −0.0474 | 0.9537 | 0.9242 |
| 14 | 14_68979835_T_C | 68979835 | 68513118 | rs11624333 | C | T | 0.2581 | −0.0911 | 0.9129 | 0.9556 |
| 14 | 14_91751788_TC_T | 91751788 | 91285444 | 14: 91751788_ | T | TC | 0.6934 | 0.0380 | 1.0387 | 1.0544 |
| | | | | TC_T | | | | | | |
| 14 | 14_91841069_A_G | 91841069 | 91374725 | rs941764 | G | A | 0.3444 | 0.0513 | 1.0526 | 1.0366 |
| 14 | 14_93070286_C_T | 93070286 | 92603941 | rs78440108 | T | C | 0.1709 | −0.0577 | 0.9439 | 0.9809 |
| 14 | 14_105213978_T_G | 105213978 | 104747641 | rs4983544 | G | T | 0.4588 | 0.0399 | 1.0407 | 1.0377 |
| 15 | 15_46680811_C_A | 46680811 | 46388613 | rs187010898 | A | C | 0.0115 | 0.1973 | 0.8209 | 0.9959 |
| 15 | 15_50694306_A_G | 50694306 | 50402109 | rs4774565 | G | A | 0.3446 | −0.0417 | 0.9592 | 0.9720 |
| 15 | 15_66630569_G_A | 66630569 | 66338231 | rs8042593 | A | G | 0.6413 | −0.0369 | 0.9638 | 0.9541 |
| 15 | 15_67457698_A_G | 67457698 | 67165360 | rs35874463 | G | A | 0.0496 | 0.0782 | 1.0813 | 1.0081 |
| 15 | 15_75750383_T_C | 75750383 | 75458042 | rs8035987 | C | T | 0.2604 | −0.0413 | 0.9595 | 0.9790 |
| 15 | 15_91512267_G_T | 91512267 | 90969037 | rs2290202 | T | G | 0.1353 | −0.0589 | 0.9428 | 0.9846 |
| 15 | 15_100905819_A_C | 100905819 | 100365614 | rs144767203 | C | A | 0.1100 | −0.0608 | 0.9410 | 0.9871 |
| 16 | 16_4008542_CAAAA | 4008542 | 3958541 | 16: 4008542_ | C | CAAAAA | 0.8213 | −0.0329 | 0.9676 | 0.9475 |
| | A_C | | | CAAAAA_C | | | | | | |
| 16 | 16_4106788_C_A | 4106788 | 4056787 | rs11076805 | A | C | 0.2643 | −0.0300 | 0.9704 | 0.9844 |
| 16 | 16_6963972_C_G | 6963972 | 6913971 | rs12709163 | G | C | 0.7835 | 0.0354 | 1.0360 | 1.0573 |
| 16 | 16_10706580_G_A | 10706580 | 10612723 | rs34872983 | A | G | 0.0695 | −0.0740 | 0.9287 | 0.9901 |
| 16 | 16_23007047_G_T | 23007047 | 22995726 | rs75753503 | T | G | 0.0236 | 0.1218 | 1.1295 | 1.0061 |
| 16 | 16_52538825_C_A | 52538825 | 52504913 | rs35668161 | A | C | 0.2562 | 0.1147 | 1.1215 | 1.0632 |
| 16 | 16_52599188_C_T | 52599188 | 52565276 | rs4784227 | T | C | 0.2406 | 0.1070 | 1.1129 | 1.0551 |
| 16 | 16_53809123_C_T | 53809123 | 53775211 | rs55872725 | T | C | 0.4201 | −0.0704 | 0.9320 | 0.9437 |
| 16 | 16_53861139_C_T | 53861139 | 53827227 | rs6499648 | T | C | 0.7604 | −0.0338 | 0.9668 | 0.9501 |
| 16 | 16_53861592_G_A | 53861592 | 53827680 | rs7184573 | A | G | 0.3663 | −0.0337 | 0.9669 | 0.9759 |
| 16 | 16_54682064_G_A | 54682064 | 54648152 | rs28539243 | A | G | 0.4850 | 0.0477 | 1.0489 | 1.0480 |
| 16 | 16_80648296_A_G | 80648296 | 80614399 | rs7500067 | G | A | 0.2303 | 0.0839 | 1.0875 | 1.0407 |
| 16 | 16_85145977_T_C | 85145977 | 85112371 | rs9931038 | C | T | 0.4856 | −0.0211 | 0.9791 | 0.9798 |
| 16 | 16_87086492_T_C | 87086492 | 87052886 | rs12449271 | C | T | 0.2586 | −0.0469 | 0.9542 | 0.9764 |
| 17 | 17_29168077_G_T | 29168077 | 30841059 | rs79461387 | T | G | 0.2613 | −0.0568 | 0.9448 | 0.9713 |
| 17 | 17_39251123_T_C | 39251123 | 41094871 | rs150537328 | C | T | 0.0682 | 0.0799 | 1.0832 | 1.0114 |
| 17 | 17_40127060_T_C | 40127060 | 41975042 | rs11296 | C | T | 0.0570 | 0.0174 | 1.0176 | 1.0020 |
| 17 | 17_40485239_G_T | 40485239 | 42333221 | rs17881320 | T | G | 0.0874 | −0.0571 | 0.9445 | 0.9903 |
| 17 | 17_40744470_G_A | 40744470 | 42592452 | rs149370081 | A | G | 0.0124 | 0.2017 | 1.2235 | 1.0055 |
| 17 | 17_43212339_C_CT | 43212339 | 45134972 | rs545502941 | CT | C | 0.2284 | 0.0438 | 1.0448 | 1.0206 |
| 17 | 17_44283858_G_A | 44283858 | 46206492 | rs2668667 | A | G | 0.1895 | 0.0540 | 0.9474 | 0.9802 |
| 17 | 17_53209774_A_C | 53209774 | 55132413 | rs2787486 | C | A | 0.3023 | −0.0793 | 0.9238 | 0.9544 |
| 17 | 17_77781725_A_G | 77781725 | 79807926 | rs745570 | G | A | 0.5038 | −0.0401 | 0.9607 | 0.9608 |
| 18 | 18_11696613_C_T | 11696613 | 11696614 | rs16976596 | T | C | 0.1379 | −0.0381 | 0.9626 | 0.9897 |
| 18 | 18_20634253_C_T | 20634253 | 23054290 | rs11665269 | T | C | 0.6403 | −0.0415 | 0.9593 | 0.9486 |
| 18 | 18_24125857_T_C | 24125857 | 26545893 | rs1111207 | C | T | 0.4214 | 0.0346 | 1.0352 | 1.0299 |
| 18 | 18_24337424_C_G | 24337424 | 26757460 | rs527616 | G | C | 0.6205 | 0.0455 | 1.0466 | 1.0586 |
| 18 | 18_24518050_AT_A | 24518050 | 26938086 | 18: 24518050_ | A | AT | 0.2773 | −0.0599 | 0.9419 | 0.9680 |
| | | | | AT_A | | | | | | |
| 18 | 18_25407513_C_G | 25407513 | 27827549 | rs8092192 | G | C | 0.7126 | 0.0399 | 1.0407 | 1.0589 |
| 18 | 18_29981526_G_A | 29981526 | 32401563 | rs72931898 | A | G | 0.0474 | −0.1058 | 0.8996 | 0.9905 |
| 18 | 18_42411803_G_C | 42411803 | 44831838 | rs9954058 | C | G | 0.0717 | −0.0877 | 0.9160 | 0.9880 |
| 18 | 18_42888797_T_C | 42888797 | 45308832 | rs9952980 | C | T | 0.3519 | −0.0542 | 0.9472 | 0.9632 |
| 19 | 19_13249921_G_T | 13249921 | 13139107 | rs117922601 | T | G | 0.0513 | 0.0956 | 1.1003 | 1.0103 |
| 19 | 19_17393925_C_A | 17393925 | 17283116 | rs56069439 | A | C | 0.2958 | 0.0378 | 1.0385 | 1.0229 |
| 19 | 19_18569492_C_T | 18569492 | 18458682 | rs10164323 | T | C | 0.3481 | −0.0719 | 0.9306 | 0.9523 |
| 19 | 19_19517054_C_ | 19517054 | 19406245 | rs140702307 | CGGGCG | C | 0.3537 | 0.0437 | 1.0447 | 1.0318 |
| | CGGGCG | | | | | | | | | |
| 19 | 19_44283031_T_C | 44283031 | 43778879 | rs56681946 | C | T | 0.3519 | 0.0619 | 1.0639 | 1.0454 |
| 19 | 19_46166073_T_C | 46166073 | 45662815 | rs4399645 | C | T | 0.6074 | −0.0360 | 0.9646 | 0.9575 |
| 19 | 19_55816678_C_T | 55816678 | 55305310 | rs1172821 | T | C | 0.3626 | −0.0359 | 0.9647 | 0.9746 |
| 20 | 20_5948227_G_A | 5948227 | 5967581 | rs16991615 | A | G | 0.0628 | 0.0760 | 1.0790 | 1.0099 |
| 20 | 20_11379842_T_C | 11379842 | 11399194 | rs1154723 | C | T | 0.9483 | 0.0844 | 1.0881 | 1.1740 |
| 20 | 20_41613706_C_G | 41613706 | 42985066 | rs6030585 | G | C | 0.7928 | 0.0315 | 1.0320 | 1.0514 |
| 20 | 20_52296849_G_A | 52296849 | 53680310 | rs13039563 | A | G | 0.2400 | 0.0440 | 1.0450 | 1.0217 |
| 21 | 21_16364756_T_G | 16364756 | 14992435 | rs2822999 | G | T | 0.1732 | 0.0646 | 1.0667 | 1.0233 |
| 21 | 21_16566350_A_G | 16566350 | 15194030 | rs2823130 | G | A | 0.0873 | 0.0595 | 1.0613 | 1.0107 |
| 21 | 21_16574455_C_A | 16574455 | 15202135 | rs2403907 | A | C | 0.3167 | −0.0707 | 0.9317 | 0.9572 |
| 21 | 21_47762932_G_A | 47762932 | 46343018 | rs4818836 | A | G | 0.0355 | 0.0946 | 1.0992 | 1.0071 |
| 22 | 22_19766137_C_T | 19766137 | 19778614 | rs9798754 | T | C | 0.3798 | −0.0367 | 0.9640 | 0.9728 |
| 22 | 22_29121087_A_G | 29121087 | 28725099 | rs17879961 | G | A | 0.0054 | 0.1839 | 1.2019 | 1.0022 |
| 22 | 22_29135543_G_A | 29135543 | 28739555 | rs5997390 | A | G | 0.0870 | 0.0654 | 1.0676 | 1.0118 |
| 22 | 22_29203724_C_T | 29203724 | 28807736 | rs34134147 | T | C | 0.0209 | 0.1405 | 1.1508 | 1.0063 |
| 22 | 22_29551872_A_G | 29551872 | 29155884 | rs132289 | G | A | 0.9846 | −0.1716 | 0.8423 | 0.7136 |
| 22 | 22_38583315_AAAA | 38583315 | 38187308 | | AAAAGA | AAAAG | 0.2805 | −0.0471 | 0.9540 | 0.9744 |
| | G_AAAAGAAAG | | | | AAG | | | | | |
| 22 | 22_39343916_T_A | 39343916 | 38947911 | rs5750715 | A | T | 0.2541 | 0.0407 | 1.0415 | 1.0212 |
| 22 | 22_40904707_CT_C | 40904707 | 40508703 | 22: 40904707_ | C | CT | 0.1099 | 0.1148 | 1.1216 | 1.0269 |
| | | | | CT_C | | | | | | |
| 22 | 22_43433100_C_T | 43433100 | 43037094 | rs9611990 | T | C | 0.1144 | −0.0600 | 0.9418 | 0.9867 |
| 22 | 22_45319953_G_A | 45319953 | 44924073 | rs112855987 | A | G | 0.4166 | −0.0134 | 0.9867 | 0.9889 |
| 22 | 22_46283297_G_A | 46283297 | 45887417 | rs28512361 | A | G | 0.1117 | 0.0736 | 1.0764 | 1.0171 |

TABLE 18
3,820 polymorphism panel.

| | | | | | | Ref- | | | OR | |
|---|---|---|---|---|---|---|---|---|---|---|
| | | GRCh37 | GRCh38 | | Ef- | er- | Effect | ß | Over- | |
| Chro- | | Posi- | Posi- | | fect | ence | Allele | Overall | all | |
| mo- | | tion | tion | | Al- | Al- | Fre- | Breast | Breast | |
| some | SNP | (hg19) | (hg38) | RS Number | lele | lele | quency | Cancer | Cancer | μ |
| 1 | 1_4693695_ | 4693695 | 4633635 | rs74051909 | T | C | 0.2718 | −0.0086 | 0.9914 | 0.9954 |
| | C_T | | | | | | | | | |
| 1 | 1_5470084_ | 5470084 | 5410024 | rs61763346 | G | A | 0.0367 | 0.0163 | 1.0164 | 1.0012 |
| | A_G | | | | | | | | | |
| 1 | 1_7598422_ | 7598422 | 7538362 | 1: | T | TA | 0.0579 | −0.0078 | 0.9922 | 0.9991 |
| | TA_T | | | 7598422_ | | | | | | |
| | | | | TA_T | | | | | | |
| 1 | 1_7799738_ | 7799738 | 7739678 | rs59214896 | G | A | 0.2085 | 0.0002 | 1.0002 | 1.0001 |
| | A_G | | | | | | | | | |
| 1 | 1_7801288_ | 7801288 | 7741228 | rs2032563 | A | G | 0.1975 | 0.0008 | 1.0008 | 1.0003 |
| | G_A | | | | | | | | | |
| 1 | 1_7911221_ | 7911221 | 7851161 | rs79479445 | G | A | 0.0630 | −0.0003 | 0.9997 | 1.0000 |
| | A_G | | | | | | | | | |
| 1 | 1_7917076_ | 7917076 | 7857016 | rs707475 | A | G | 0.3899 | −0.0137 | 0.9864 | 0.9894 |
| | G_A | | | | | | | | | |
| 1 | 1_8095500_ | 8095500 | 8035440 | rs225132 | G | T | 0.1821 | −0.0132 | 0.9869 | 0.9952 |
| | T_G | | | | | | | | | |
| 1 | 1_9280040_ | 9280040 | 9219981 | rs10864397 | T | C | 0.5749 | 0.0039 | 1.0039 | 1.0045 |
| | C_T | | | | | | | | | |
| 1 | 1_10315413_ | 10315413 | 10255355 | rs112432872 | A | G | 0.0514 | 0.0000 | 1.0000 | 1.0000 |
| | G_A | | | | | | | | | |
| 1 | 1_10525841_ | 10525841 | 10465784 | rs34132699 | C | T | 0.2568 | 0.0000 | 1.0000 | 1.0000 |
| | T_C | | | | | | | | | |
| 1 | 1_10554505_ | 10554505 | 10494448 | rs597438 | G | A | 0.7029 | 0.0050 | 1.0050 | 1.0071 |
| | A_G | | | | | | | | | |
| 1 | 1_10565488_ | 10565488 | 10505431 | rs1755594 | T | C | 0.6348 | 0.0000 | 1.0000 | 1.0000 |
| | C_T | | | | | | | | | |
| 1 | 1_10566215_ | 10566215 | 10506158 | rs616488 | G | A | 0.3290 | −0.0246 | 0.9757 | 0.9841 |
| | A_G | | | | | | | | | |
| 1 | 1_10570721_ | 10570721 | 10510664 | rs2480784 | A | G | 0.0676 | −0.0038 | 0.9962 | 0.9995 |
| | G_A | | | | | | | | | |
| 1 | 1_10612820_ | 10612820 | 10552763 | rs9430149 | C | G | 0.3149 | −0.0010 | 0.9990 | 0.9994 |
| | G_C | | | | | | | | | |
| 1 | 1_10614233_ | 10614233 | 10554176 | rs35612158 | T | C | 0.1803 | 0.0062 | 1.0062 | 1.0022 |
| | C_T | | | | | | | | | |
| 1 | 1_10629772_ | 10629772 | 10569715 | rs76431309 | T | G | 0.0536 | 0.0000 | 1.0000 | 1.0000 |
| | G_T | | | | | | | | | |
| 1 | 1_10633489_ | 10633489 | 10573432 | rs78507554 | G | A | 0.1476 | 0.0000 | 1.0000 | 1.0000 |
| | A_G | | | | | | | | | |
| 1 | 1_10704688_ | 10704688 | 10644631 | rs11587145 | T | C | 0.1333 | 0.0083 | 1.0083 | 1.0022 |
| | C_T | | | | | | | | | |
| 1 | 1_11843224_ | 11843224 | 11783167 | rs116276371 | T | C | 0.0265 | 0.0054 | 1.0054 | 1.0003 |
| | C_T | | | | | | | | | |
| 1 | 1_14507831_ | 14507831 | 14181336 | rs12065331 | T | C | 0.3179 | 0.0063 | 1.0063 | 1.0040 |
| | C_T | | | | | | | | | |
| 1 | 1_15296282_ | 15296282 | 14969786 | rs4575078 | G | T | 0.2643 | 0.0067 | 1.0067 | 1.0036 |
| | T_G | | | | | | | | | |
| 1 | 1_15693556_ | 15693556 | 15367060 | rs2496346 | T | A | 0.1854 | 0.0035 | 1.0035 | 1.0013 |
| | A_T | | | | | | | | | |
| 1 | 1_15719028_ | 15719028 | 15392532 | rs12734304 | G | A | 0.1302 | 0.0059 | 1.0059 | 1.0015 |
| | A_G | | | | | | | | | |
| 1 | 1_16075981_ | 16075981 | 15749486 | rs11585750 | T | C | 0.3254 | 0.0086 | 1.0086 | 1.0056 |
| | C_T | | | | | | | | | |
| 1 | 1_17733996_ | 17733996 | 17407500 | rs3193024 | A | G | 0.1119 | −0.0077 | 0.9923 | 0.9983 |
| | G_A | | | | | | | | | |
| 1 | 1_18800547_ | 18800547 | 18474053 | rs2992734 | G | A | 0.7428 | 0.0000 | 1.0000 | 1.0000 |
| | A_G | | | | | | | | | |
| 1 | 1_18807339_ | 18807339 | 18480845 | rs2992756 | C | T | 0.5145 | −0.0098 | 0.9902 | 0.9900 |
| | T_C | | | | | | | | | |
| 1 | 1_18807953_ | 18807953 | 18481459 | rs11261022 | A | C | 0.3691 | −0.0097 | 0.9903 | 0.9929 |
| | C_A | | | | | | | | | |
| 1 | 1_18808292_ | 18808292 | 18481798 | rs2992753 | A | C | 0.6357 | −0.0172 | 0.9829 | 0.9784 |
| | C_A | | | | | | | | | |
| 1 | 1_18809916_ | 18809916 | 18483422 | rs12061708 | A | G | 0.3226 | −0.0070 | 0.9930 | 0.9955 |
| | G_A | | | | | | | | | |
| 1 | 1_21262731_ | 21262731 | 20936238 | rs72652973 | A | G | 0.0113 | −0.0196 | 0.9806 | 0.9996 |
| | G_A | | | | | | | | | |
| 1 | 1_22370457_ | 22370457 | 22043964 | 1:22370457_ | C | CT | 0.1770 | −0.0010 | 0.9990 | 0.9996 |
| | CT_C | | | CT_C | | | | | | |
| 1 | 1_22439520_ | 22439520 | 22113027 | rs4654783 | C | T | 0.6978 | 0.0044 | 1.0044 | 1.0062 |
| | T_C | | | | | | | | | |
| 1 | 1_22485467_ | 22485467 | 22158974 | rs2807365 | G | A | 0.3350 | −0.0021 | 0.9979 | 0.9986 |
| | A_G | | | | | | | | | |
| 1 | 1_22585468_ | 22585468 | 22258975 | rs76843148 | A | G | 0.0560 | 0.0057 | 1.0057 | 1.0006 |
| | G_A | | | | | | | | | |
| 1 | 1_23068465_ | 23068465 | 22741972 | rs6669666 | T | C | 0.2068 | −0.0028 | 0.9972 | 0.9988 |
| | C_T | | | | | | | | | |
| 1 | 1_23098679_ | 23098679 | 22772186 | rs114804938 | T | C | 0.0189 | 0.0203 | 1.0205 | 1.0008 |
| | C_T | | | | | | | | | |
| 1 | 1_23862653_ | 23862653 | 23536161 | rs760607 | G | A | 0.3939 | 0.0065 | 1.0065 | 1.0051 |
| | A_G | | | | | | | | | |
| 1 | 1_24565083_ | 24565083 | 24238593 | rs11249049 | T | C | 0.2619 | −0.0075 | 0.9925 | 0.9961 |
| | C_T | | | | | | | | | |
| 1 | 1_24593061_ | 24593061 | 24266571 | rs72874872 | C | T | 0.0425 | 0.0033 | 1.0033 | 1.0003 |
| | T_C | | | | | | | | | |
| 1 | 1_25197155_ | 25197155 | 24870664 | rs4648881 | A | G | 0.4848 | 0.0032 | 1.0032 | 1.0031 |
| | G_A | | | | | | | | | |
| 1 | 1_25220498_ | 25220498 | 24894007 | rs11249197 | T | G | 0.4802 | 0.0000 | 1.0000 | 1.0000 |
| | G_T | | | | | | | | | |
| 1 | 1_25241056_ | 25241056 | 24914565 | rs2282718 | A | G | 0.3977 | −0.0123 | 0.9878 | 0.9903 |
| | G_A | | | | | | | | | |
| 1 | 1_25251706_ | 25251706 | 24925215 | rs742232 | C | T | 0.5531 | 0.0006 | 1.0006 | 1.0007 |
| | T_C | | | | | | | | | |
| 1 | 1_26758602_ | 26758602 | 26432111 | rs3811462 | C | T | 0.7850 | 0.0052 | 1.0052 | 1.0082 |
| | T_C | | | | | | | | | |
| 1 | 1_27304568_ | 27304568 | 26978077 | rs2993063 | G | A | 0.7725 | −0.0001 | 0.9999 | 0.9998 |
| | A_G | | | | | | | | | |
| 1 | 1_27557607_ | 27557607 | 27231116 | rs4609452 | A | G | 0.8161 | −0.0069 | 0.9931 | 0.9888 |
| | G_A | | | | | | | | | |
| 1 | 1_32407833_ | 32407833 | 31942232 | rs139994937 | T | C | 0.0612 | −0.0242 | 0.9761 | 0.9971 |
| | C_T | | | | | | | | | |
| 1 | 1_32551080_ | 32551080 | 32085479 | rs72666734 | A | G | 0.0631 | −0.0108 | 0.9893 | 0.9986 |
| | G_A | | | | | | | | | |
| 1 | 1_33727981_ | 33727981 | 33262380 | rs34542197 | CT | C | 0.7619 | 0.0126 | 1.0127 | 1.0194 |
| | C_CT | | | | | | | | | |
| 1 | 1_35619907_ | 35619907 | 35154306 | rs2878436 | A | G | 0.8952 | −0.0151 | 0.9850 | 0.9733 |
| | G_A | | | | | | | | | |
| 1 | 1_36588402_ | 36588402 | 36122801 | rs11263857 | A | G | 0.1021 | 0.0108 | 1.0109 | 1.0022 |
| | G_A | | | | | | | | | |
| 1 | 1_39976128_ | 39976128 | 39510456 | rs1180325 | C | T | 0.4332 | −0.0039 | 0.9961 | 0.9966 |
| | T_C | | | | | | | | | |
| 1 | 1_40398728_ | 40398728 | 39933056 | rs7554235 | T | C | 0.2564 | 0.0081 | 1.0081 | 1.0042 |
| | C_T | | | | | | | | | |
| 1 | 1_40435999_ | 40435999 | 39970327 | rs2131311 | G | A | 0.7259 | −0.0053 | 0.9947 | 0.9923 |
| | A_G | | | | | | | | | |
| 1 | 1_41332388_ | 41332388 | 40866716 | rs12049459 | T | C | 0.3185 | 0.0065 | 1.0065 | 1.0042 |
| | C_T | | | | | | | | | |
| 1 | 1_41351322_ | 41351322 | 40885650 | rs111473215 | G | A | 0.3118 | −0.0049 | 0.9951 | 0.9970 |
| | A_G | | | | | | | | | |
| 1 | 1_41380170_ | 41380170 | 40914498 | rs769225960 | AG | A | 0.8630 | 0.0010 | 1.0010 | 1.0017 |
| | A_AG | | | | | | | | | |
| 1 | 1_41380440_ | 41380440 | 40914768 | rs4233486 | T | C | 0.6438 | 0.0157 | 1.0158 | 1.0205 |
| | C_T | | | | | | | | | |
| 1 | 1_41389220_ | 41389220 | 40923548 | rs114282204 | C | T | 0.0169 | 0.0531 | 1.0545 | 1.0018 |
| | T_C | | | | | | | | | |
| 1 | 1_41887180_ | 41887180 | 41421508 | 1:41887180_ | G | GT | 0.5612 | −0.0035 | 0.9965 | 0.9961 |
| | GT_G | | | GT_G | | | | | | |
| 1 | 1_41909856_ | 41909856 | 41444185 | rs7534137 | T | C | 0.1359 | 0.0037 | 1.0037 | 1.0010 |
| | C_T | | | | | | | | | |
| 1 | 1_42018135_ | 42018135 | 41552464 | rs144427343 | A | G | 0.0478 | 0.0048 | 1.0048 | 1.0005 |
| | G_A | | | | | | | | | |
| 1 | 1_42058260_ | 42058260 | 41592589 | rs116351921 | C | G | 0.0777 | 0.0204 | 1.0206 | 1.0032 |
| | G_C | | | | | | | | | |
| 1 | 1_42140601_ | 42140601 | 41674930 | rs56394244 | T | C | 0.0336 | −0.0415 | 0.9593 | 0.9973 |
| | C_T | | | | | | | | | |
| 1 | 1_42176600_ | 42176600 | 41710929 | rs36021639 | T | C | 0.0841 | 0.0005 | 1.0005 | 1.0001 |
| | C_T | | | | | | | | | |
| 1 | 1_42204512_ | 42204512 | 41738841 | rs77615557 | G | A | 0.0567 | 0.0158 | 1.0159 | 1.0018 |
| | A_G | | | | | | | | | |
| 1 | 1_44514883_ | 44514883 | 44049211 | rs2108201 | C | G | 0.6371 | −0.0083 | 0.9917 | 0.9895 |
| | G_C | | | | | | | | | |
| 1 | 1_44564405_ | 44564405 | 44098733 | rs12756908 | A | G | 0.3317 | −0.0022 | 0.9978 | 0.9985 |
| | G_A | | | | | | | | | |
| 1 | 1_44926646_ | 44926646 | 44460974 | rs946960 | C | T | 0.2722 | 0.0000 | 1.0000 | 1.0000 |
| | T_C | | | | | | | | | |
| 1 | 1_44935232_ | 44935232 | 44469560 | rs35387836 | TATTC | T | 0.3280 | −0.0017 | 0.9983 | 0.9989 |
| | T_TATTC | | | | | | | | | |
| 1 | 1_45842878_ | 45842878 | 45377206 | rs76179004 | G | C | 0.0470 | −0.0018 | 0.9982 | 0.9998 |
| | C_G | | | | | | | | | |
| 1 | 1_45939591_ | 45939591 | 45473919 | rs1765709 | T | C | 0.4998 | 0.0000 | 1.0000 | 1.0000 |
| | C_T | | | | | | | | | |
| 1 | 1_46487168_ | 46487168 | 46021496 | rs946526 | C | T | 0.9570 | 0.0101 | 1.0102 | 1.0195 |
| | T_C | | | | | | | | | |
| 1 | 1_46526185_ | 46526185 | 46060513 | rs72677528 | G | A | 0.0604 | 0.0012 | 1.0012 | 1.0001 |
| | A_G | | | | | | | | | |
| 1 | 1_46600917_ | 46600917 | 46135245 | rs1707302 | G | A | 0.6633 | 0.0073 | 1.0073 | 1.0097 |
| | A_G | | | | | | | | | |
| 1 | 1_46670206_ | 46670206 | 46204534 | 1:46670206_ | T | TC | 0.2973 | 0.0050 | 1.0050 | 1.0030 |
| | TC_T | | | TC_T | | | | | | |
| 1 | 1_46701282_ | 46701282 | 46235610 | rs12129542 | A | G | 0.7452 | 0.0000 | 1.0000 | 1.0000 |
| | G_A | | | | | | | | | |
| 1 | 1_46776759_ | 46776759 | 46311087 | rs61273183 | T | A | 0.5999 | 0.0000 | 1.0000 | 1.0000 |
| | A_T | | | | | | | | | |
| 1 | 1_46823917_ | 46823917 | 46358245 | rs6674538 | G | A | 0.2891 | 0.0003 | 1.0003 | 1.0002 |
| | A_G | | | | | | | | | |
| 1 | 1_46842079_ | 46842079 | 46376407 | 1:46842079_ | T | TA | 0.3065 | 0.0195 | 1.0197 | 1.0121 |
| | TA_T | | | TA_T | | | | | | |
| 1 | 1_50771882_ | 50771882 | 50306210 | rs12070259 | C | T | 0.0459 | 0.0093 | 1.0093 | 1.0009 |
| | T_C | | | | | | | | | |
| 1 | 1_51129510_ | 51129510 | 50663838 | 1:51129510_ | A | AATC | 0.0324 | 0.0157 | 1.0158 | 1.0010 |
| | AATC_A | | | AATC_A | | | | | | |
| 1 | 1_51467096_ | 51467096 | 51001424 | 1:51467096_ | C | CT | 0.4800 | 0.0057 | 1.0057 | 1.0055 |
| | CT_C | | | CT_C | | | | | | |
| 1 | 1_51478336_ | 51478336 | 51012664 | rs35311109 | A | T | 0.2917 | 0.0077 | 1.0077 | 1.0045 |
| | T_A | | | | | | | | | |
| 1 | 1_56363704_ | 56363704 | 55898031 | 1:56363704_ | C | CT | 0.2643 | 0.0053 | 1.0053 | 1.0028 |
| | CT_C | | | CT_C | | | | | | |
| 1 | 1_58592530_ | 58592530 | 58126858 | rs6659357 | T | C | 0.2020 | 0.0031 | 1.0031 | 1.0013 |
| | C_T | | | | | | | | | |
| 1 | 1_58891310_ | 58891310 | 58425638 | rs724564 | G | A | 0.1220 | −0.0176 | 0.9826 | 0.9957 |
| | A_G | | | | | | | | | |
| 1 | 1_59981754_ | 59981754 | 59516082 | 1:59981754_ | A | ATTA | 0.1190 | 0.0000 | 1.0000 | 1.0000 |
| | ATTATGCCTTC_ | | | ATTATGCCTTC_ | | TGCC | | | | |
| | A | | | A | | TTC | | | | |
| 1 | 1_61000020_ | 61000020 | 60534348 | rs142036730 | C | A | 0.0155 | −0.0201 | 0.9801 | 0.9994 |
| | A_C | | | | | | | | | |
| 1 | 1_61681136_ | 61681136 | 61215464 | 1:61681136_ | T | TA | 0.0830 | −0.0112 | 0.9889 | 0.9982 |
| | TA_T | | | TA_T | | | | | | |
| 1 | 1_61894902_ | 61894902 | 61429230 | rs2207791 | G | A | 0.4727 | −0.0002 | 0.9998 | 0.9998 |
| | A_G | | | | | | | | | |
| 1 | 1_61895257_ | 61895257 | 61429585 | rs6587924 | A | C | 0.5013 | 0.0000 | 1.0000 | 1.0000 |
| | C_A | | | | | | | | | |
| 1 | 1_61911157_ | 61911157 | 61445485 | rs2261980 | A | G | 0.2298 | 0.0054 | 1.0054 | 1.0025 |
| | G_A | | | | | | | | | |
| 1 | 1_62382617_ | 62382617 | 61916945 | rs6587949 | C | A | 0.6255 | 0.0093 | 1.0093 | 1.0117 |
| | A_C | | | | | | | | | |
| 1 | 1_62445706_ | 62445706 | 61980034 | rs885581 | G | A | 0.4111 | −0.0065 | 0.9935 | 0.9947 |
| | A_G | | | | | | | | | |
| 1 | 1_63635787_ | 63635787 | 63170116 | rs71813788 | CA | C | 0.0876 | 0.0035 | 1.0035 | 1.0006 |
| | C_CA | | | | | | | | | |
| 1 | 1_63638791_ | 63638791 | 63173120 | rs6588016 | A | G | 0.5140 | −0.0034 | 0.9966 | 0.9965 |
| | G_A | | | | | | | | | |
| 1 | 1_63818393_ | 63818393 | 63352722 | rs6675284 | T | C | 0.4145 | −0.0036 | 0.9964 | 0.9970 |
| | C_T | | | | | | | | | |
| 1 | 1_64124310_ | 64124310 | 63658639 | rs59784071 | A | T | 0.0437 | 0.0000 | 1.0000 | 1.0000 |
| | T_A | | | | | | | | | |
| 1 | 1_64536417_ | 64536417 | 64070745 | rs113619373 | A | G | 0.0207 | 0.0179 | 1.0181 | 1.0007 |
| | G_A | | | | | | | | | |
| 1 | 1_66128239_ | 66128239 | 65662556 | rs6664626 | T | G | 0.1402 | 0.0074 | 1.0074 | 1.0021 |
| | G_T | | | | | | | | | |
| 1 | 1_66341400_ | 66341400 | 65875717 | rs12759968 | T | C | 0.1740 | 0.0040 | 1.0040 | 1.0014 |
| | C_T | | | | | | | | | |
| 1 | 1_66359367_ | 66359367 | 65893684 | rs375308081 | CATATAT | C | 0.2583 | 0.0032 | 1.0032 | 1.0017 |
| | C_CATATATAT | | | | AT | | | | | |
| 1 | 1_66389097_ | 66389097 | 65923414 | rs4655809 | T | C | 0.3597 | 0.0086 | 1.0086 | 1.0062 |
| | C_T | | | | | | | | | |
| 1 | 1_69746553_ | 69746553 | 69280870 | rs114230792 | G | A | 0.0387 | 0.0072 | 1.0072 | 1.0006 |
| | A_G | | | | | | | | | |
| 1 | 1_69881944_ | 69881944 | 69416261 | rs12027709 | T | G | 0.1287 | 0.0096 | 1.0096 | 1.0025 |
| | G_T | | | | | | | | | |
| 1 | 1_72002778_ | 72002778 | 71537095 | rs78725171 | C | T | 0.0555 | 0.0135 | 1.0136 | 1.0015 |
| | T_C | | | | | | | | | |
| 1 | 1_72511514_ | 72511514 | 72045831 | rs764149303 | A | AT | 0.2675 | −0.0042 | 0.9958 | 0.9978 |
| | AT_A | | | | | | | | | |
| 1 | 1_76067119_ | 76067119 | 75601434 | 1:76067119_ | A | ATGA | 0.2029 | 0.0083 | 1.0083 | 1.0034 |
| | ATGACGGGT_A | | | ATGACGGGT_A | | CGGG | | | | |
| | | | | | | T | | | | |
| 1 | 1_76073887_ | 76073887 | 75608202 | rs148880761 | CA | C | 0.2091 | 0.0028 | 1.0028 | 1.0012 |
| | C_CA | | | | | | | | | |
| 1 | 1_76962558_ | 76962558 | 76496873 | rs75325512 | G | C | 0.0503 | −0.0152 | 0.9849 | 0.9985 |
| | C_G | | | | | | | | | |
| 1 | 1_78464526_ | 78464526 | 77998842 | | CTAAA | C | 0.7818 | −0.0026 | 0.9974 | 0.9959 |
| | C_CTAAA | | | | | | | | | |
| 1 | 1_81567342_ | 81567342 | 81101657 | rs10518651 | C | T | 0.0694 | −0.0107 | 0.9894 | 0.9985 |
| | T_C | | | | | | | | | |
| 1 | 1_84223168_ | 84223168 | 83757485 | rs6663583 | G | A | 0.8653 | 0.0123 | 1.0124 | 1.0215 |
| | A_G | | | | | | | | | |
| 1 | 1_84265575_ | 84265575 | 83799892 | rs761405 | T | G | 0.5554 | 0.0056 | 1.0056 | 1.0062 |
| | G_T | | | | | | | | | |
| 1 | 1_86474446_ | 86474446 | 86008763 | rs58845753 | T | C | 0.0245 | −0.0207 | 0.9795 | 0.9990 |
| | C_T | | | | | | | | | |
| 1 | 1_87688394_ | 87688394 | 87222711 | rs10873824 | G | A | 0.7330 | 0.0093 | 1.0093 | 1.0137 |
| | A_G | | | | | | | | | |
| 1 | 1_87849060_ | 87849060 | 87383377 | 1:87849060_ | T | TA | 0.7309 | −0.0046 | 0.9954 | 0.9933 |
| | TA_T | | | TA_T | | | | | | |
| 1 | 1_87850495_ | 87850495 | 87384812 | rs1415174 | C | T | 0.7559 | 0.0000 | 1.0000 | 1.0000 |
| | T_C | | | | | | | | | |
| 1 | 1_87856591_ | 87856591 | 87390908 | | C | CT | 0.1534 | −0.0106 | 0.9895 | 0.9968 |
| | CT_C | | | | | | | | | |
| 1 | 1_87890934_ | 87890934 | 87425251 | rs144893360 | TACCAGT | T | 0.3945 | 0.0107 | 1.0108 | 1.0085 |
| | T_TACCAGTGC | | | | GCCTTCT | | | | | |
| | CTTCTCTTGAG | | | | CTTGAGA | | | | | |
| | AATGCAGCTAA | | | | ATGCAGC | | | | | |
| | ACAC | | | | TAAACAC | | | | | |
| 1 | 1_88119395_ | 88119395 | 87653712 | rs28404364 | C | T | 0.6191 | 0.0067 | 1.0067 | 1.0083 |
| | T_C | | | | | | | | | |
| 1 | 1_88156923_ | 88156923 | 87691240 | rs17426269 | A | G | 0.1487 | 0.0171 | 1.0172 | 1.0051 |
| | G_A | | | | | | | | | |
| 1 | 1_88189692_ | 88189692 | 87724009 | rs305461 | G | C | 0.2365 | 0.0032 | 1.0032 | 1.0015 |
| | C_G | | | | | | | | | |
| 1 | 1_88207421_ | 88207421 | 87741738 | rs6700541 | G | A | 0.1774 | 0.0000 | 1.0000 | 1.0000 |
| | A_G | | | | | | | | | |
| 1 | 1_88343731_ | 88343731 | 87878048 | rs143643147 | GGTGTT | G | 0.0452 | 0.0157 | 1.0158 | 1.0014 |
| | G_GGTGTT | | | | | | | | | |
| 1 | 1_88428199_ | 88428199 | 87962516 | rs2151842 | A | C | 0.2477 | −0.0149 | 0.9852 | 0.9927 |
| | C_A | | | | | | | | | |
| 1 | 1_88429257_ | 88429257 | 87963574 | rs3008452 | C | T | 0.4701 | −0.0028 | 0.9972 | 0.9974 |
| | T_C | | | | | | | | | |
| 1 | 1_88764449_ | 88764449 | 88298766 | rs17326377 | T | C | 0.0400 | −0.0131 | 0.9870 | 0.9990 |
| | C_T | | | | | | | | | |
| 1 | 1_90602741_ | 90602741 | 90137183 | rs12567673 | T | A | 0.1726 | 0.0025 | 1.0025 | 1.0009 |
| | A_T | | | | | | | | | |
| 1 | 1_92273253_ | 92273253 | 91807696 | rs284156 | G | C | 0.4087 | 0.0070 | 1.0070 | 1.0058 |
| | C_G | | | | | | | | | |
| 1 | 1_93811023_ | 93811023 | 93345466 | rs76909542 | A | G | 0.0122 | −0.0182 | 0.9820 | 0.9996 |
| | G_A | | | | | | | | | |
| 1 | 1_95028119_ | 95028119 | 94562563 | rs2022032 | T | C | 0.2719 | 0.0008 | 1.0008 | 1.0004 |
| | C_T | | | | | | | | | |
| 1 | 1_96786288_ | 96786288 | 96320732 | rs113194393 | T | C | 0.0152 | 0.0053 | 1.0053 | 1.0002 |
| | C_T | | | | | | | | | |
| 1 | 1_97032073_ | 97032073 | 96566517 | rs755722246 | T | TA | 0.0113 | 0.0142 | 1.0143 | 1.0003 |
| | TA_T | | | | | | | | | |
| 1 | 1_99933338_ | 99933338 | 99467782 | rs12144940 | C | T | 0.2423 | 0.0045 | 1.0045 | 1.0022 |
| | T_C | | | | | | | | | |
| 1 | 1_100781169_ | 100781169 | 100315613 | rs3119188 | C | T | 0.1472 | 0.0031 | 1.0031 | 1.0009 |
| | T_C | | | | | | | | | |
| 1 | 1_100880328_ | 100880328 | 100414772 | rs612683 | T | A | 0.4097 | 0.0148 | 1.0149 | 1.0123 |
| | A_T | | | | | | | | | |
| 1 | 1_101687757_ | 101687757 | 101222201 | rs10875362 | A | G | 0.4019 | 0.0085 | 1.0085 | 1.0069 |
| | G_A | | | | | | | | | |
| 1 | 1_101702825_ | 101702825 | 101237269 | rs3753194 | G | A | 0.0588 | 0.0128 | 1.0129 | 1.0015 |
| | A_G | | | | | | | | | |
| 1 | 1_102596334_ | 102596334 | 102130778 | rs34470656 | C | T | 0.2019 | −0.0065 | 0.9935 | 0.9974 |
| | T_C | | | | | | | | | |
| 1 | 1_102718900_ | 102718900 | 102253344 | rs77712548 | C | G | 0.0103 | 0.0331 | 1.0337 | 1.0007 |
| | G_C | | | | | | | | | |
| 1 | 1_107295378_ | 107295378 | 106752756 | rs74979491 | T | C | 0.0322 | −0.0101 | 0.9900 | 0.9994 |
| | C_T | | | | | | | | | |
| 1 | 1_108657357_ | 108657357 | 108114735 | | ATT | A | 0.8258 | −0.0098 | 0.9902 | 0.9840 |
| | A_ATT | | | | | | | | | |
| 1 | 1_109692641_ | 109692641 | 109150019 | rs610249 | A | G | 0.5630 | 0.0037 | 1.0037 | 1.0042 |
| | G_A | | | | | | | | | |
| 1 | 1_109725239_ | 109725239 | 109182617 | rs621414 | T | C | 0.5694 | 0.0022 | 1.0022 | 1.0025 |
| | C_T | | | | | | | | | |
| 1 | 1_109727653_ | 109727653 | 109185031 | 1:109727653_ | C | CT | 0.1273 | −0.0152 | 0.9849 | 0.9962 |
| | CT_C | | | CT_C | | | | | | |
| 1 | 1_110093159_ | 110093159 | 109550537 | rs370352103 | CT | C | 0.1534 | −0.0079 | 0.9921 | 0.9976 |
| | C_CT | | | | | | | | | |
| 1 | 1_110195901_ | 110195901 | 109653279 | rs115450622 | C | T | 0.0242 | −0.0093 | 0.9907 | 0.9996 |
| | T_C | | | | | | | | | |
| 1 | 1_110198129_ | 110198129 | 109655507 | 1:110198129_ | C | CAAA | 0.7755 | 0.0155 | 1.0156 | 1.0244 |
| | CAAA_C | | | CAAA_C | | | | | | |
| 1 | 1_110212534_ | 110212534 | 109669912 | rs687643 | C | T | 0.4872 | 0.0077 | 1.0077 | 1.0075 |
| | T_C | | | | | | | | | |
| 1 | 1_110218716_ | 110218716 | 109676094 | rs620077 | T | C | 0.9133 | 0.0042 | 1.0042 | 1.0077 |
| | C_T | | | | | | | | | |
| 1 | 1_112949872_ | 112949872 | 112407250 | rs72697071 | T | C | 0.0767 | 0.0039 | 1.0039 | 1.0006 |
| | C_T | | | | | | | | | |
| 1 | 1_114096460_ | 114096460 | 113553838 | rs4274063 | G | A | 0.3074 | 0.0003 | 1.0003 | 1.0002 |
| | A_G | | | | | | | | | |
| 1 | 1_114173410_ | 114173410 | 113630788 | rs1230666 | G | A | 0.8723 | −0.0122 | 0.9879 | 0.9790 |
| | A_G | | | | | | | | | |
| 1 | 1_114422976_ | 114422976 | 113880354 | | A | T | 0.7454 | −0.0036 | 0.9964 | 0.9947 |
| | T_A | | | | | | | | | |
| 1 | 1_114445880_ | 114445880 | 113903258 | rs7513707 | A | G | 0.1664 | 0.0249 | 1.0252 | 1.0084 |
| | G_A | | | | | | | | | |
| 1 | 1_114813581_ | 114813581 | 114270959 | rs7541732 | A | G | 0.1149 | −0.0019 | 0.9981 | 0.9996 |
| | G_A | | | | | | | | | |
| 1 | 1_114939944_ | 114939944 | 114397322 | rs35834438 | G | A | 0.0599 | −0.0191 | 0.9811 | 0.9977 |
| | A_G | | | | | | | | | |
| 1 | 1_116286496_ | 116286496 | 115743875 | rs145128079 | T | G | 0.0106 | −0.0090 | 0.9910 | 0.9998 |
| | G_T | | | | | | | | | |
| 1 | 1_116296125_ | 116296125 | 115753504 | rs11440911 | CA | C | 0.9776 | −0.0215 | 0.9787 | 0.9588 |
| | C_CA | | | | | | | | | |
| 1 | 1_116304301_ | 116304301 | 115761680 | rs12023881 | T | G | 0.3231 | 0.0030 | 1.0030 | 1.0019 |
| | G_T | | | | | | | | | |
| 1 | 1_116310967_ | 116310967 | 115768346 | rs4074536 | C | T | 0.2852 | 0.0015 | 1.0015 | 1.0009 |
| | T_C | | | | | | | | | |
| 1 | 1_116807794_ | 116807794 | 116265172 | rs6699946 | T | C | 0.6082 | −0.0081 | 0.9919 | 0.9902 |
| | C_T | | | | | | | | | |
| 1 | 1_118141340_ | 118141340 | 117598718 | rs12731593 | A | T | 0.2288 | 0.0015 | 1.0015 | 1.0007 |
| | T_A | | | | | | | | | |
| 1 | 1_118141492_ | 118141492 | 117598870 | rs12406858 | C | A | 0.2657 | 0.0112 | 1.0113 | 1.0060 |
| | A_C | | | | | | | | | |
| 1 | 1_118195233_ | 118195233 | 117652611 | rs319497 | C | T | 0.2691 | 0.0096 | 1.0096 | 1.0052 |
| | T_C | | | | | | | | | |
| 1 | 1_118238892_ | 118238892 | 117696270 | rs61416637 | GT | G | 0.3530 | −0.0025 | 0.9975 | 0.9982 |
| | G_GT | | | | | | | | | |
| 1 | 1_118942949_ | 118942949 | 118400326 | rs61837306 | C | G | 0.1407 | −0.0112 | 0.9889 | 0.9969 |
| | G_C | | | | | | | | | |
| 1 | 1_119030698_ | 119030698 | 118488075 | rs10923574 | C | A | 0.3256 | −0.0078 | 0.9922 | 0.9949 |
| | A_C | | | | | | | | | |
| 1 | 1_120257110_ | 120257110 | 119714487 | rs637868 | C | T | 0.5309 | 0.0114 | 1.0115 | 1.0122 |
| | T_C | | | | | | | | | |
| 1 | 1_120261388_ | 120261388 | 119718765 | rs839616 | T | G | 0.3904 | 0.0004 | 1.0004 | 1.0003 |
| | G_T | | | | | | | | | |
| 1 | 1_120385937_ | 120385937 | 119843314 | rs2994823 | G | C | 0.0800 | −0.0079 | 0.9921 | 0.9987 |
| | C_G | | | | | | | | | |
| 1 | 1_120392239_ | 120392239 | 119849616 | rs12734865 | G | C | 0.0700 | −0.0068 | 0.9932 | 0.9991 |
| | C_G | | | | | | | | | |
| 1 | 1_120436751_ | 120436751 | 119894128 | rs35273427 | C | T | 0.0594 | 0.0311 | 1.0316 | 1.0038 |
| | T_C | | | | | | | | | |
| 1 | 1_121232045_ | 121232045 | 121490247 | rs116450554 | T | A | 0.0641 | −0.0251 | 0.9752 | 0.9968 |
| | A_T | | | | | | | | | |
| 1 | 1_121280613_ | 121280613 | 121538815 | rs11249433 | G | A | 0.4053 | 0.0597 | 1.0615 | 1.0505 |
| | A_G | | | | | | | | | |
| 1 | 1_121287994_ | 121287994 | 121546196 | rs111458676 | G | A | 0.1060 | −0.0341 | 0.9665 | 0.9929 |
| | A_G | | | | | | | | | |
| 1 | 1_121288049_ | 121288049 | 121546251 | rs12135688 | G | A | 0.0939 | 0.0000 | 1.0000 | 1.0000 |
| | A_G | | | | | | | | | |
| 1 | 1_121288913_ | 121288913 | 121547115 | rs11249430 | G | C | 0.4982 | 0.0056 | 1.0056 | 1.0056 |
| | C_G | | | | | | | | | |
| 1 | 1_121343930_ | 121343930 | 121602132 | rs146695762 | C | G | 0.0346 | 0.0190 | 1.0192 | 1.0013 |
| | G_C | | | | | | | | | |
| 1 | 1_121347829_ | 121347829 | 121606031 | rs12733576 | G | A | 0.0115 | −0.0018 | 0.9982 | 1.0000 |
| | A_G | | | | | | | | | |
| 1 | 1_145365316_ | 145365316 | 146069686 | rs61813408 | A | G | 0.3721 | −0.0119 | 0.9882 | 0.9912 |
| | G_A | | | | | | | | | |
| 1 | 1_145577585_ | 145577585 | 145857483 | rs12756687 | G | C | 0.5936 | −0.0034 | 0.9966 | 0.9960 |
| | C_G | | | | | | | | | |
| 1 | 1_145604302_ | 145604302 | 145830811 | rs143384623 | CT | C | 0.3515 | −0.0148 | 0.9853 | 0.9897 |
| | C_CT | | | | | | | | | |
| 1 | 1_145604791_ | 145604791 | 145830322 | rs36107432 | T | G | 0.3692 | −0.0002 | 0.9998 | 0.9999 |
| | G_T | | | | | | | | | |
| 1 | 1_145661932_ | 145661932 | 145773146 | rs35353761 | G | A | 0.2419 | −0.0020 | 0.9980 | 0.9990 |
| | A_G | | | | | | | | | |
| 1 | 1_149892872_ | 149892872 | 149920979 | rs11205277 | G | A | 0.4313 | 0.0049 | 1.0049 | 1.0042 |
| | A_G | | | | | | | | | |
| 1 | 1_149906413_ | 149906413 | 149934520 | rs11205303 | C | T | 0.4017 | 0.0321 | 1.0326 | 1.0264 |
| | T_C | | | | | | | | | |
| 1 | 1_151328794_ | 151328794 | 151356318 | rs744536 | A | T | 0.6283 | 0.0034 | 1.0034 | 1.0043 |
| | T_A | | | | | | | | | |
| 1 | 1_155046402_ | 155046402 | 155073926 | rs72702268 | T | G | 0.0179 | −0.0294 | 0.9710 | 0.9990 |
| | G_T | | | | | | | | | |
| 1 | 1_155123837_ | 155123837 | 155151361 | rs6676150 | C | G | 0.3965 | 0.0000 | 1.0000 | 1.0000 |
| | G_C | | | | | | | | | |
| 1 | 1_155155608_ | 155155608 | 155183132 | rs4971099 | G | A | 0.4411 | 0.0000 | 1.0000 | 1.0000 |
| | A_G | | | | | | | | | |
| 1 | 1_155184975_ | 155184975 | 155215184 | rs2990223 | G | A | 0.5932 | 0.0095 | 1.0095 | 1.0114 |
| | A_G | | | | | | | | | |
| 1 | 1_155195071_ | 155195071 | 155225280 | rs139558368 | A | ATTA | 0.4418 | −0.0009 | 0.9991 | 0.9992 |
| | ATTAT_A | | | | | T | | | | |
| 1 | 1_155219452_ | 155219452 | 155249661 | rs147869881 | T | C | 0.0303 | 0.0365 | 1.0372 | 1.0023 |
| | C_T | | | | | | | | | |
| 1 | 1_155358506_ | 155358506 | 155388715 | 1:155358506_ | C | CT | 0.3305 | 0.0001 | 1.0001 | 1.0001 |
| | CT_C | | | CT_C | | | | | | |
| 1 | 1_155556971_ | 155556971 | 155587180 | rs12091730 | A | G | 0.2302 | 0.0196 | 1.0198 | 1.0091 |
| | G_A | | | | | | | | | |
| 1 | 1_155848692_ | 155848692 | 155878901 | rs2025575 | A | T | 0.7961 | 0.0000 | 1.0000 | 1.0000 |
| | T_A | | | | | | | | | |
| 1 | 1_156146546_ | 156146546 | 156176755 | rs76381440 | T | C | 0.0155 | −0.0054 | 0.9946 | 0.9998 |
| | C_T | | | | | | | | | |
| 1 | 1_156153043_ | 156153043 | 156183252 | rs11264454 | G | A | 0.4240 | 0.0098 | 1.0098 | 1.0084 |
| | A_G | | | | | | | | | |
| 1 | 1_156158777_ | 156158777 | 156188986 | rs114417629 | A | G | 0.1410 | 0.0042 | 1.0042 | 1.0012 |
| | G_A | | | | | | | | | |
| 1 | 1_156187024_ | 156187024 | 156217233 | 1:156187024_ | T | TA | 0.3352 | 0.0002 | 1.0002 | 1.0001 |
| | TA_T | | | TA_T | | | | | | |
| 1 | 1_156662700_ | 156662700 | 156692908 | rs114082130 | A | G | 0.0529 | 0.0071 | 1.0071 | 1.0008 |
| | G_A | | | | | | | | | |
| 1 | 1_156768568_ | 156768568 | 156798776 | rs150411804 | A | G | 0.0368 | 0.0333 | 1.0339 | 1.0025 |
| | G_A | | | | | | | | | |
| 1 | 1_157224242_ | 157224242 | 157254452 | rs190475459 | A | C | 0.0132 | −0.0371 | 0.9636 | 0.9990 |
| | C_A | | | | | | | | | |
| 1 | 1_157634687_ | 157634687 | 157664897 | rs150038704 | C | A | 0.0279 | 0.0221 | 1.0223 | 1.0012 |
| | A_C | | | | | | | | | |
| 1 | 1_157873941_ | 157873941 | 157904151 | | CAAAA | CA | 0.0295 | −0.0093 | 0.9907 | 0.9995 |
| | CA_CAAAA | | | | | | | | | |
| 1 | 1_165885282_ | 165885282 | 165916045 | rs35899976 | A | G | 0.2257 | 0.0110 | 1.0111 | 1.0050 |
| | G_A | | | | | | | | | |
| 1 | 1_167820337_ | 167820337 | 167851099 | rs149612953 | G | T | 0.0501 | −0.0016 | 0.9984 | 0.9998 |
| | T_G | | | | | | | | | |
| 1 | 1_167918583_ | 167918583 | 167949345 | rs145940992 | C | T | 0.0183 | −0.0028 | 0.9972 | 0.9999 |
| | T_C | | | | | | | | | |
| 1 | 1_168086743_ | 168086743 | 168117505 | rs4657734 | T | C | 0.3464 | −0.0085 | 0.9915 | 0.9941 |
| | C_T | | | | | | | | | |
| 1 | 1_168095701_ | 168095701 | 168126463 | rs138538350 | GT | G | 0.1269 | −0.0091 | 0.9909 | 0.9977 |
| | G_GT | | | | | | | | | |
| 1 | 1_168171052_ | 168171052 | 168201814 | rs761575824 | C | CA | 0.1097 | −0.0332 | 0.9673 | 0.9929 |
| | CA_C | | | | | | | | | |
| 1 | 1_168539624_ | 168539624 | 168570386 | rs7354947 | C | G | 0.3904 | 0.0032 | 1.0032 | 1.0025 |
| | G_C | | | | | | | | | |
| 1 | 1_168542393_ | 168542393 | 168573155 | rs55748465 | C | T | 0.1069 | 0.0112 | 1.0113 | 1.0024 |
| | T_C | | | | | | | | | |
| 1 | 1_169180229_ | 169180229 | 169210991 | | C | CT | 0.4365 | −0.0036 | 0.9964 | 0.9969 |
| | CT_C | | | | | | | | | |
| 1 | 1_169457028_ | 169457028 | 169487790 | rs12138875 | G | A | 0.3873 | −0.0031 | 0.9969 | 0.9976 |
| | A_G | | | | | | | | | |
| 1 | 1_169460645_ | 169460645 | 169491407 | rs12143031 | G | A | 0.1902 | 0.0005 | 1.0005 | 1.0002 |
| | A_G | | | | | | | | | |
| 1 | 1_169574722_ | 169574722 | 169605484 | rs2205894 | G | T | 0.3179 | 0.0036 | 1.0036 | 1.0023 |
| | T_G | | | | | | | | | |
| 1 | 1_171131262_ | 171131262 | 171162123 | rs148297052 | TAAAAA | T | 0.0372 | 0.0079 | 1.0079 | 1.0006 |
| | T_TAAAAA | | | | | | | | | |
| 1 | 1_172328767_ | 172328767 | 172359627 | rs11463354 | TA | T | 0.3305 | −0.0147 | 0.9854 | 0.9904 |
| | T_TA | | | | | | | | | |
| 1 | 1_172331059_ | 172331059 | 172361919 | rs1894633 | G | A | 0.3305 | −0.0026 | 0.9974 | 0.9983 |
| | A_G | | | | | | | | | |
| 1 | 1_172415351_ | 172415351 | 172446211 | rs200139225 | G | GA | 0.5047 | 0.0013 | 1.0013 | 1.0013 |
| | GA_G | | | | | | | | | |
| 1 | 1_172452463_ | 172452463 | 172483323 | | C | CAG | 0.6726 | 0.0071 | 1.0071 | 1.0096 |
| | CAG_C | | | | | | | | | |
| 1 | 1_177902387_ | 177902387 | 177933252 | rs61745560 | A | C | 0.2483 | 0.0010 | 1.0010 | 1.0005 |
| | C_A | | | | | | | | | |
| 1 | 1_177903281_ | 177903281 | 177934146 | rs500142 | A | T | 0.6890 | −0.0054 | 0.9946 | 0.9926 |
| | T_A | | | | | | | | | |
| 1 | 1_183773076_ | 183773076 | 183803942 | rs3814327 | G | A | 0.3331 | 0.0052 | 1.0052 | 1.0035 |
| | A_G | | | | | | | | | |
| 1 | 1_186732170_ | 186732170 | 186763038 | rs56160832 | A | G | 0.1097 | 0.0052 | 1.0052 | 1.0011 |
| | G_A | | | | | | | | | |
| 1 | 1_186787603_ | 186787603 | 186818471 | 1:186787603_ | C | CGTG | 0.1264 | 0.0034 | 1.0034 | 1.0009 |
| | CGTGTGTGTGT_ | | | CGTGTGTGTG | | TGTG | | | | |
| | C | | | T_C | | TGT | | | | |
| 1 | 1_187858446_ | 187858446 | 187889315 | rs800807 | A | G | 0.7220 | 0.0008 | 1.0008 | 1.0012 |
| | G_A | | | | | | | | | |
| 1 | 1_187884585_ | 187884585 | 187915454 | rs1548791 | T | C | 0.7875 | 0.0071 | 1.0071 | 1.0113 |
| | C_T | | | | | | | | | |
| 1 | 1_188666269_ | 188666269 | 188697138 | rs28819695 | A | G | 0.4820 | −0.0030 | 0.9970 | 0.9971 |
| | G_A | | | | | | | | | |
| 1 | 1_188700606_ | 188700606 | 188731475 | rs73055028 | C | G | 0.4309 | −0.0066 | 0.9934 | 0.9943 |
| | G_C | | | | | | | | | |
| 1 | 1_193938198_ | 193938198 | 193969068 | rs657308 | G | A | 0.6068 | 0.0000 | 1.0000 | 1.0000 |
| | A_G | | | | | | | | | |
| 1 | 1_199438261_ | 199438261 | 199469133 | 1:199438261_ | T | TAA | 0.5507 | −0.0020 | 0.9980 | 0.9978 |
| | TAA_T | | | TAA_T | | | | | | |
| 1 | 1_199679378_ | 199679378 | 199710250 | rs17699358 | G | A | 0.3911 | 0.0078 | 1.0078 | 1.0061 |
| | A_G | | | | | | | | | |
| 1 | 1_201437832_ | 201437832 | 201468704 | rs35383942 | T | C | 0.0559 | 0.0357 | 1.0363 | 1.0041 |
| | C_T | | | | | | | | | |
| 1 | 1_201630517_ | 201630517 | 201661389 | rs724221 | A | G | 0.4446 | −0.0103 | 0.9898 | 0.9909 |
| | G_A | | | | | | | | | |
| 1 | 1_201662222_ | 201662222 | 201693094 | rs2007411 | G | A | 0.4454 | 0.0018 | 1.0018 | 1.0016 |
| | A_G | | | | | | | | | |
| 1 | 1_202184600_ | 202184600 | 202215472 | rs6686987 | T | C | 0.3992 | 0.0000 | 1.0000 | 1.0000 |
| | C_T | | | | | | | | | |
| 1 | 1_202196697_ | 202196697 | 202227569 | rs12032080 | A | G | 0.4011 | 0.0000 | 1.0000 | 1.0000 |
| | G_A | | | | | | | | | |
| 1 | 1_202442574_ | 202442574 | 202473446 | rs3767418 | A | G | 0.0597 | 0.0305 | 1.0310 | 1.0037 |
| | G_A | | | | | | | | | |
| 1 | 1_202570918_ | 202570918 | 202601790 | rs61820902 | A | G | 0.0563 | 0.0150 | 1.0151 | 1.0017 |
| | G_A | | | | | | | | | |
| 1 | 1_203770448_ | 203770448 | 203801320 | rs7514172 | A | T | 0.2715 | 0.0157 | 1.0158 | 1.0086 |
| | T_A | | | | | | | | | |
| 1 | 1_203842355_ | 203842355 | 203873227 | | A | T | 0.1268 | 0.0074 | 1.0074 | 1.0019 |
| | T_A | | | | | | | | | |
| 1 | 1_204061744_ | 204061744 | 204092616 | rs7528560 | A | G | 0.1270 | −0.0011 | 0.9989 | 0.9997 |
| | G_A | | | | | | | | | |
| 1 | 1_204423559_ | 204423559 | 204454431 | rs151283122 | A | G | 0.0169 | −0.0332 | 0.9673 | 0.9989 |
| | G_A | | | | | | | | | |
| 1 | 1_204490311_ | 204490311 | 204521183 | rs11586387 | T | A | 0.0165 | 0.0144 | 1.0145 | 1.0005 |
| | A_T | | | | | | | | | |
| 1 | 1_204491551_ | 204491551 | 204522423 | rs35946963 | CAG | C | 0.7402 | 0.0000 | 1.0000 | 1.0000 |
| | C_CAG | | | | | | | | | |
| 1 | 1_205556426_ | 205556426 | 205587298 | rs6593954 | C | T | 0.3123 | −0.0024 | 0.9976 | 0.9985 |
| | T_C | | | | | | | | | |
| 1 | 1_208076291_ | 208076291 | 207902946 | rs2785646 | A | G | 0.3337 | −0.0095 | 0.9905 | 0.9937 |
| | G_A | | | | | | | | | |
| 1 | 1_208142894_ | 208142894 | 207969549 | rs35303635 | G | A | 0.3694 | −0.0004 | 0.9996 | 0.9997 |
| | A_G | | | | | | | | | |
| 1 | 1_210042456_ | 210042456 | 209869111 | rs11119358 | C | T | 0.1594 | 0.0000 | 1.0000 | 1.0000 |
| | T_C | | | | | | | | | |
| 1 | 1_210042983_ | 210042983 | 209869638 | rs2206043 | A | G | 0.1460 | 0.0000 | 1.0000 | 1.0000 |
| | G_A | | | | | | | | | |
| 1 | 1_210050756_ | 210050756 | 209877411 | rs76610459 | G | T | 0.1567 | 0.0000 | 1.0000 | 1.0000 |
| | T_G | | | | | | | | | |
| 1 | 1_210796591_ | 210796591 | 210623247 | rs7548424 | C | T | 0.9069 | 0.0153 | 1.0154 | 1.0282 |
| | T_C | | | | | | | | | |
| 1 | 1_211828151_ | 211828151 | 211654809 | rs7526473 | A | G | 0.4251 | −0.0075 | 0.9925 | 0.9937 |
| | G_A | | | | | | | | | |
| 1 | 1_213722114_ | 213722114 | 213548771 | rs320394 | C | T | 0.8421 | 0.0082 | 1.0082 | 1.0139 |
| | T_C | | | | | | | | | |
| 1 | 1_214154719_ | 214154719 | 213981376 | rs2075423 | T | G | 0.3536 | 0.0086 | 1.0086 | 1.0061 |
| | G_T | | | | | | | | | |
| 1 | 1_214524613_ | 214524613 | 214351270 | rs7414346 | C | T | 0.5900 | −0.0040 | 0.9960 | 0.9953 |
| | T_C | | | | | | | | | |
| 1 | 1_214623189_ | 214623189 | 214449846 | | TAAAA | TAAA | 0.4513 | −0.0098 | 0.9902 | 0.9912 |
| | TAAAAAA_ | | | | AAA | AAA | | | | |
| | TAAAAAAA | | | | | | | | | |
| 1 | 1_215226642_ | 215226642 | 215053299 | rs12118830 | A | T | 0.0413 | 0.0355 | 1.0361 | 1.0030 |
| | T_A | | | | | | | | | |
| 1 | 1_216871412_ | 216871412 | 216698070 | rs17043393 | G | A | 0.1576 | 0.0059 | 1.0059 | 1.0019 |
| | A_G | | | | | | | | | |
| 1 | 1_217053815_ | 217053815 | 216880473 | rs2576261 | G | T | 0.3280 | 0.0179 | 1.0181 | 1.0119 |
| | T_G | | | | | | | | | |
| 1 | 1_217200883_ | 217200883 | 217027541 | rs2377360 | A | G | 0.7529 | 0.0000 | 1.0000 | 1.0000 |
| | G_A | | | | | | | | | |
| 1 | 1_217220574_ | 217220574 | 217047232 | rs11117758 | A | G | 0.2107 | −0.0075 | 0.9925 | 0.9969 |
| | G_A | | | | | | | | | |
| 1 | 1_217236895_ | 217236895 | 217063553 | rs1416526 | G | T | 0.1565 | −0.0017 | 0.9983 | 0.9995 |
| | T_G | | | | | | | | | |
| 1 | 1_219168079_ | 219168079 | 218994737 | rs67056850 | T | A | 0.3367 | −0.0034 | 0.9966 | 0.9977 |
| | A_T | | | | | | | | | |
| 1 | 1_220583106_ | 220583106 | 220409764 | rs1890461 | C | A | 0.7727 | −0.0097 | 0.9903 | 0.9851 |
| | A_C | | | | | | | | | |
| 1 | 1_220675924_ | 220675924 | 220502582 | rs11118564 | T | G | 0.6814 | −0.0022 | 0.9978 | 0.9970 |
| | G_T | | | | | | | | | |
| 1 | 1_220684965_ | 220684965 | 220511623 | rs11417702 | AT | A | 0.7491 | −0.0020 | 0.9980 | 0.9970 |
| | A_AT | | | | | | | | | |
| 1 | 1_220731475_ | 220731475 | 220558133 | rs369215403 | A | AAAT | 0.7125 | −0.0033 | 0.9967 | 0.9953 |
| | AAAT_A | | | | | | | | | |
| 1 | 1_220824115_ | 220824115 | 220650773 | rs12123306 | C | G | 0.4324 | −0.0016 | 0.9984 | 0.9986 |
| | G_C | | | | | | | | | |
| 1 | 1_221436185_ | 221436185 | 221262843 | rs9431466 | G | C | 0.6529 | −0.0010 | 0.9990 | 0.9987 |
| | C_G | | | | | | | | | |
| 1 | 1_221898651_ | 221898651 | 221725309 | rs116232610 | G | A | 0.0209 | 0.0202 | 1.0204 | 1.0009 |
| | A_G | | | | | | | | | |
| 1 | 1_223922062_ | 223922062 | 223734360 | rs7517684 | G | A | 0.1398 | 0.0027 | 1.0027 | 1.0008 |
| | A_G | | | | | | | | | |
| 1 | 1_223937520_ | 223937520 | 223749818 | rs12563713 | A | C | 0.0825 | 0.0084 | 1.0084 | 1.0014 |
| | C_A | | | | | | | | | |
| 1 | 1_224180489_ | 224180489 | 223992787 | rs11799363 | T | C | 0.2025 | 0.0096 | 1.0096 | 1.0039 |
| | C_T | | | | | | | | | |
| 1 | 1_228178296_ | 228178296 | 227990595 | rs112479896 | G | T | 0.0664 | −0.0032 | 0.9968 | 0.9996 |
| | T_G | | | | | | | | | |
| 1 | 1_228856469_ | 228856469 | 228720722 | rs76436368 | T | C | 0.0757 | −0.0043 | 0.9957 | 0.9994 |
| | C_T | | | | | | | | | |
| 1 | 1_228974769_ | 228974769 | 228839022 | rs1389138 | T | C | 0.1384 | 0.0000 | 1.0000 | 1.0000 |
| | C_T | | | | | | | | | |
| 1 | 1_229934838_ | 229934838 | 229799091 | rs581850 | T | A | 0.5172 | 0.0000 | 1.0000 | 1.0000 |
| | A_T | | | | | | | | | |
| 1 | 1_230622709_ | 230622709 | 230486963 | rs853022 | T | C | 0.7638 | 0.0000 | 1.0000 | 1.0000 |
| | C_T | | | | | | | | | |
| 1 | 1_230849190_ | 230849190 | 230713444 | rs7539020 | T | C | 0.3710 | −0.0060 | 0.9940 | 0.9956 |
| | C_T | | | | | | | | | |
| 1 | 1_230851628_ | 230851628 | 230715882 | rs6683321 | A | G | 0.1760 | −0.0021 | 0.9979 | 0.9993 |
| | G_A | | | | | | | | | |
| 1 | 1_230852551_ | 230852551 | 230716805 | rs1977414 | T | C | 0.2840 | −0.0027 | 0.9973 | 0.9985 |
| | C_T | | | | | | | | | |
| 1 | 1_231711128_ | 231711128 | 231575382 | 1:231711128_ | A | AT | 0.1655 | 0.0105 | 1.0106 | 1.0035 |
| | AT_A | | | AT_A | | | | | | |
| 1 | 1_231974202_ | 231974202 | 231838456 | rs76945478 | G | T | 0.0377 | −0.0093 | 0.9907 | 0.9993 |
| | T_G | | | | | | | | | |
| 1 | 1_232002834_ | 232002834 | 231867088 | rs55696805 | C | T | 0.0417 | −0.0055 | 0.9945 | 0.9995 |
| | T_C | | | | | | | | | |
| 1 | 1_232004691_ | 232004691 | 231868945 | rs9432019 | T | G | 0.6021 | −0.0099 | 0.9901 | 0.9882 |
| | G_T | | | | | | | | | |
| 1 | 1_232235709_ | 232235709 | 232099963 | rs12146088 | A | G | 0.0186 | 0.0045 | 1.0045 | 1.0002 |
| | G_A | | | | | | | | | |
| 1 | 1_232908177_ | 232908177 | 232772431 | rs2814544 | A | C | 0.0756 | −0.0183 | 0.9819 | 0.9973 |
| | C_A | | | | | | | | | |
| 1 | 1_233060511_ | 233060511 | 232924765 | rs1555394 | C | T | 0.2302 | −0.0102 | 0.9899 | 0.9953 |
| | T_C | | | | | | | | | |
| 1 | 1_233063389_ | 233063389 | 232927643 | rs7546587 | T | C | 0.2488 | −0.0003 | 0.9997 | 0.9999 |
| | C_T | | | | | | | | | |
| 1 | 1_233476712_ | 233476712 | 233340966 | rs4649217 | C | T | 0.3304 | −0.0078 | 0.9922 | 0.9949 |
| | T_C | | | | | | | | | |
| 1 | 1_233560466_ | 233560466 | 233424720 | rs6692313 | G | C | 0.7789 | −0.0129 | 0.9872 | 0.9801 |
| | C_G | | | | | | | | | |
| 1 | 1_233717882_ | 233717882 | 233582136 | rs4649337 | T | C | 0.0121 | 0.0173 | 1.0175 | 1.0004 |
| | C_T | | | | | | | | | |
| 1 | 1_240217696_ | 240217696 | 240054396 | rs75280320 | G | C | 0.2445 | −0.0098 | 0.9902 | 0.9952 |
| | C_G | | | | | | | | | |
| 1 | 1_242034263_ | 242034263 | 241870961 | rs72755295 | G | A | 0.0305 | 0.0897 | 1.0938 | 1.0057 |
| | A_G | | | | | | | | | |
| 1 | 1_242047847_ | 242047847 | 241884545 | rs4149991 | G | A | 0.1061 | 0.0017 | 1.0017 | 1.0004 |
| | A_G | | | | | | | | | |
| 1 | 1_243143385_ | 243143385 | 242980083 | rs7417508 | A | G | 0.3746 | −0.0005 | 0.9995 | 0.9996 |
| | G_A | | | | | | | | | |
| 1 | 1_243487861_ | 243487861 | 243324559 | rs12058508 | A | G | 0.2760 | 0.0018 | 1.0018 | 1.0010 |
| | G_A | | | | | | | | | |
| 1 | 1_243492643_ | 243492643 | 243329341 | rs2490400 | T | C | 0.7709 | 0.0076 | 1.0076 | 1.0118 |
| | C_T | | | | | | | | | |
| 1 | 1_244150083_ | 244150083 | 243986781 | rs472967 | T | C | 0.5380 | −0.0045 | 0.9955 | 0.9952 |
| | C_T | | | | | | | | | |
| 1 | 1_247358075_ | 247358075 | 247194773 | rs12136392 | T | C | 0.1513 | 0.0017 | 1.0017 | 1.0005 |
| | C_T | | | | | | | | | |
| 1 | 1_247772275_ | 247772275 | 247608973 | rs80281736 | C | G | 0.2255 | −0.0035 | 0.9965 | 0.9984 |
| | G_C | | | | | | | | | |
| 2 | 2_609177_ | 609177 | 609177 | rs2947415 | G | A | 0.8259 | 0.0000 | 1.0000 | 1.0000 |
| | A_G | | | | | | | | | |
| 2 | 2_620989_ | 620989 | 620989 | rs6753148 | A | G | 0.6244 | 0.0000 | 1.0000 | 1.0000 |
| | G_A | | | | | | | | | |
| 2 | 2_621954_ | 621954 | 621954 | rs1320332 | C | T | 0.8229 | −0.0191 | 0.9811 | 0.9691 |
| | T_C | | | | | | | | | |
| 2 | 2_1047592_ | 1047592 | 1051906 | rs114056577 | AACA | CACA | 0.0198 | 0.0095 | 1.0095 | 1.0004 |
| | CACA_AACA | | | | | | | | | |
| 2 | 2_1140472_ | 1140472 | 1144786 | rs7584432 | T | C | 0.3275 | −0.0079 | 0.9921 | 0.9949 |
| | C_T | | | | | | | | | |
| 2 | 2_1821846_ | 1821846 | 1818074 | rs34526459 | G | C | 0.2901 | 0.0015 | 1.0015 | 1.0009 |
| | C_G | | | | | | | | | |
| 2 | 2_3408669_ | 3408669 | 3404898 | rs7355558 | A | G | 0.1233 | 0.0073 | 1.0073 | 1.0018 |
| | G_A | | | | | | | | | |
| 2 | 2_5495963_ | 5495963 | 5355830 | rs11677035 | T | C | 0.4933 | 0.0057 | 1.0057 | 1.0056 |
| | C_T | | | | | | | | | |
| 2 | 2_6121326_ | 6121326 | 5981194 | 2:6121326_AA | A | AACA | 0.7392 | −0.0036 | 0.9964 | 0.9947 |
| | AACACACACAC_ | | | CACACACAC_A | | CACA | | | | |
| | A | | | | | CAC | | | | |
| 2 | 2_7188646_ | 7188646 | 7048515 | rs2460410 | C | T | 0.5504 | −0.0008 | 0.9992 | 0.9991 |
| | T_C | | | | | | | | | |
| 2 | 2_7202628_ | 7202628 | 7062497 | rs762471000 | T | TA | 0.4727 | −0.0054 | 0.9946 | 0.9949 |
| | TA_T | | | | | | | | | |
| 2 | 2_9841449_ | 9841449 | 9701320 | rs12613334 | C | T | 0.5896 | 0.0038 | 1.0038 | 1.0045 |
| | T_C | | | | | | | | | |
| 2 | 2_10028920_ | 10028920 | 9888791 | rs1620983 | G | T | 0.6215 | −0.0041 | 0.9959 | 0.9949 |
| | T_G | | | | | | | | | |
| 2 | 2_10081789_ | 10081789 | 9941660 | rs76907460 | G | A | 0.2294 | −0.0131 | 0.9870 | 0.9940 |
| | A_G | | | | | | | | | |
| 2 | 2_10109664_ | 10109664 | 9969535 | rs17364282 | G | A | 0.0145 | −0.0289 | 0.9715 | 0.9992 |
| | A_G | | | | | | | | | |
| 2 | 2_10125007_ | 10125007 | 9984879 | rs115538162 | C | T | 0.0337 | −0.0024 | 0.9976 | 0.9998 |
| | T_C | | | | | | | | | |
| 2 | 2_10138983_ | 10138983 | 9998855 | rs78425380 | C | T | 0.1160 | 0.0245 | 1.0248 | 1.0058 |
| | T_C | | | | | | | | | |
| 2 | 2_10619416_ | 10619416 | 10479290 | rs113100467 | CATAT | C | 0.1082 | 0.0091 | 1.0091 | 1.0020 |
| | C_CATAT | | | | | | | | | |
| 2 | 2_11118159_ | 11118159 | 10978033 | rs6432156 | T | A | 0.0162 | −0.0281 | 0.9723 | 0.9991 |
| | A_T | | | | | | | | | |
| 2 | 2_11681043_ | 11681043 | 11540917 | rs13021546 | T | G | 0.7171 | 0.0017 | 1.0017 | 1.0024 |
| | G_T | | | | | | | | | |
| 2 | 2_11701843_ | 11701843 | 11561717 | rs113084984 | A | G | 0.2301 | −0.0026 | 0.9974 | 0.9988 |
| | G_A | | | | | | | | | |
| 2 | 2_12035555_ | 12035555 | 11895429 | rs6432241 | C | G | 0.6899 | −0.0047 | 0.9953 | 0.9935 |
| | G_C | | | | | | | | | |
| 2 | 2_13083482_ | 13083482 | 12943356 | rs779385 | A | T | 0.1642 | 0.0007 | 1.0007 | 1.0002 |
| | T_A | | | | | | | | | |
| 2 | 2_13281213_ | 13281213 | 13141088 | rs112377288 | C | T | 0.0860 | −0.0051 | 0.9949 | 0.9991 |
| | T_C | | | | | | | | | |
| 2 | 2_13619816_ | 13619816 | 13479691 | rs6432387 | G | A | 0.3212 | 0.0052 | 1.0052 | 1.0034 |
| | A_G | | | | | | | | | |
| 2 | 2_14594985_ | 14594985 | 14454861 | rs6758768 | G | C | 0.4516 | −0.0074 | 0.9926 | 0.9934 |
| | C_G | | | | | | | | | |
| 2 | 2_17275801_ | 17275801 | 17094534 | rs4832704 | T | C | 0.0812 | 0.0149 | 1.0150 | 1.0024 |
| | C_T | | | | | | | | | |
| 2 | 2_18033944_ | 18033944 | 17852677 | rs60957847 | A | G | 0.1479 | −0.0033 | 0.9967 | 0.9990 |
| | G_A | | | | | | | | | |
| 2 | 2_18081917_ | 18081917 | 17900650 | rs146177537 | T | C | 0.0513 | −0.0040 | 0.9960 | 0.9996 |
| | C_T | | | | | | | | | |
| 2 | 2_18898271_ | 18898271 | 18717005 | rs12996938 | A | G | 0.0544 | 0.0136 | 1.0137 | 1.0015 |
| | G_A | | | | | | | | | |
| 2 | 2_18936729_ | 18936729 | 18755463 | 2:18936729_G | G | GTAT | 0.6657 | −0.0073 | 0.9927 | 0.9903 |
| | GTATATTA_G | | | TATATTA_G | | ATTA | | | | |
| 2 | 2_19297503_ | 19297503 | 19097742 | rs151210220 | TAACA | T | 0.3769 | −0.0054 | 0.9946 | 0.9959 |
| | T_TAACA | | | | | | | | | |
| 2 | 2_19302429_ | 19302429 | 19102668 | rs4283375 | A | G | 0.5597 | −0.0005 | 0.9995 | 0.9994 |
| | G_A | | | | | | | | | |
| 2 | 2_19315675_ | 19315675 | 19115914 | rs6743383 | A | T | 0.5599 | −0.0037 | 0.9963 | 0.9959 |
| | T_A | | | | | | | | | |
| 2 | 2_19320803_ | 19320803 | 19121042 | rs12710696 | C | T | 0.6341 | 0.0000 | 1.0000 | 1.0000 |
| | T_C | | | | | | | | | |
| 2 | 2_20406245_ | 20406245 | 20206484 | rs3771254 | T | C | 0.0887 | −0.0208 | 0.9794 | 0.9964 |
| | C_T | | | | | | | | | |
| 2 | 2_20592798_ | 20592798 | 20393037 | rs343155 | A | G | 0.2576 | 0.0139 | 1.0140 | 1.0072 |
| | G_A | | | | | | | | | |
| 2 | 2_20769210_ | 20769210 | 20569450 | rs57203893 | T | G | 0.0535 | 0.0165 | 1.0166 | 1.0018 |
| | G_T | | | | | | | | | |
| 2 | 2_23361864_ | 23361864 | 23138993 | rs188194202 | C | T | 0.0123 | −0.0137 | 0.9864 | 0.9997 |
| | T_C | | | | | | | | | |
| 2 | 2_25129473_ | 25129473 | 24906604 | rs6725517 | G | A | 0.4082 | −0.0174 | 0.9828 | 0.9860 |
| | A_G | | | | | | | | | |
| 2 | 2_25155419_ | 25155419 | 24932550 | rs75503394 | T | C | 0.0874 | −0.0043 | 0.9957 | 0.9993 |
| | C_T | | | | | | | | | |
| 2 | 2_25266649_ | 25266649 | 25043780 | rs190535084 | TC | T | 0.1964 | −0.0022 | 0.9978 | 0.9991 |
| | T_TC | | | | | | | | | |
| 2 | 2_25400559_ | 25400559 | 25177690 | rs13022041 | T | G | 0.3258 | 0.0000 | 1.0000 | 1.0000 |
| | G_T | | | | | | | | | |
| 2 | 2_25455389_ | 25455389 | 25232520 | rs752208 | A | G | 0.2052 | −0.0021 | 0.9979 | 0.9991 |
| | G_A | | | | | | | | | |
| 2 | 2_25473311_ | 25473311 | 25250442 | rs13427672 | A | G | 0.4313 | 0.0059 | 1.0059 | 1.0051 |
| | G_A | | | | | | | | | |
| 2 | 2_25485152_ | 25485152 | 25262283 | rs58552784 | G | C | 0.2523 | 0.0054 | 1.0054 | 1.0027 |
| | C_G | | | | | | | | | |
| 2 | 2_25526632_ | 25526632 | 25303763 | rs34635462 | T | C | 0.3970 | −0.0105 | 0.9896 | 0.9917 |
| | C_T | | | | | | | | | |
| 2 | 2_25605972_ | 25605972 | 25383103 | rs35010208 | T | C | 0.2664 | 0.0000 | 1.0000 | 1.0000 |
| | C_T | | | | | | | | | |
| 2 | 2_26109750_ | 26109750 | 25886881 | | T | TTTT | 0.7152 | 0.0063 | 1.0063 | 1.0091 |
| | TTTTA_T | | | | | A | | | | |
| 2 | 2_28549701_ | 28549701 | 28326834 | rs2002406 | G | C | 0.0863 | 0.0000 | 1.0000 | 1.0000 |
| | C_G | | | | | | | | | |
| 2 | 2_29045868_ | 29045868 | 28823002 | 2:29045868_C | C | CTT | 0.0820 | 0.0000 | 1.0000 | 1.0000 |
| | CTT_C | | | TT_C | | | | | | |
| 2 | 2_29179452_ | 29179452 | 28956586 | rs12472404 | C | G | 0.2287 | 0.0000 | 1.0000 | 1.0000 |
| | G_C | | | | | | | | | |
| 2 | 2_29198141_ | 29198141 | 28975275 | rs11674944 | G | A | 0.2340 | 0.0000 | 1.0000 | 1.0000 |
| | A_G | | | | | | | | | |
| 2 | 2_29542948_ | 29542948 | 29320082 | rs4666201 | T | C | 0.7034 | −0.0031 | 0.9969 | 0.9957 |
| | C_T | | | | | | | | | |
| 2 | 2_29586343_ | 29586343 | 29363477 | rs13010090 | A | C | 0.2468 | −0.0009 | 0.9991 | 0.9996 |
| | C_A | | | | | | | | | |
| 2 | 2_29593022_ | 29593022 | 29370156 | 2:29593022_A | A | AATC | 0.0489 | −0.0043 | 0.9957 | 0.9996 |
| | AATC_A | | | ATC_A | | | | | | |
| 2 | 2_29604977_ | 29604977 | 29382111 | rs7608573 | T | G | 0.2548 | −0.0078 | 0.9922 | 0.9960 |
| | G_T | | | | | | | | | |
| 2 | 2_29615233_ | 29615233 | 29392367 | rs4322799 | C | T | 0.2622 | −0.0098 | 0.9902 | 0.9949 |
| | T_C | | | | | | | | | |
| 2 | 2_31597032_ | 31597032 | 31374166 | rs12712316 | A | C | 0.0236 | 0.0036 | 1.0036 | 1.0002 |
| | C_A | | | | | | | | | |
| 2 | 2_32769544_ | 32769544 | 32544477 | 2:32769544_T | T | TTTT | 0.0148 | −0.0064 | 0.9936 | 0.9998 |
| | TTTTC_T | | | TTTC_T | | C | | | | |
| 2 | 2_32907914_ | 32907914 | 32682847 | rs61397127 | T | G | 0.8131 | 0.0020 | 1.0020 | 1.0033 |
| | G_T | | | | | | | | | |
| 2 | 2_32969116_ | 32969116 | 32744049 | rs3769581 | C | T | 0.2696 | 0.0019 | 1.0019 | 1.0010 |
| | T_C | | | | | | | | | |
| 2 | 2_33074113_ | 33074113 | 32849046 | rs72789809 | C | T | 0.0647 | −0.0091 | 0.9909 | 0.9988 |
| | T_C | | | | | | | | | |
| 2 | 2_33441564_ | 33441564 | 33216497 | rs17012691 | C | G | 0.0249 | 0.0202 | 1.0204 | 1.0010 |
| | G_C | | | | | | | | | |
| 2 | 2_33621242_ | 33621242 | 33396175 | rs555691297 | GT | G | 0.1296 | 0.0019 | 1.0019 | 1.0005 |
| | G_GT | | | | | | | | | |
| 2 | 2_33625577_ | 33625577 | 33400510 | 2:33625577_A | A | AGTT | 0.1364 | 0.0004 | 1.0004 | 1.0001 |
| | AGTTTTTGGTA | | | GTTTTTGGTAC | | TTTG | | | | |
| | CTTT_A | | | TTT_A | | GTAC | | | | |
| | | | | | | TTT | | | | |
| 2 | 2_33629266_ | 33629266 | 33404199 | rs148685563 | A | C | 0.0144 | 0.0203 | 1.0205 | 1.0006 |
| | C_A | | | | | | | | | |
| 2 | 2_34387201_ | 34387201 | 34162134 | rs77303967 | G | C | 0.0136 | −0.0445 | 0.9565 | 0.9988 |
| | C_G | | | | | | | | | |
| 2 | 2_39699510_ | 39699510 | 39472369 | rs553796823 | CT | C | 0.4659 | −0.0147 | 0.9854 | 0.9865 |
| | C_CT | | | | | | | | | |
| 2 | 2_40527128_ | 40527128 | 40299988 | rs727477 | G | T | 0.3029 | −0.0098 | 0.9902 | 0.9941 |
| | T_G | | | | | | | | | |
| 2 | 2_40555273_ | 40555273 | 40328133 | rs72943146 | T | G | 0.0310 | 0.0152 | 1.0153 | 1.0009 |
| | G_T | | | | | | | | | |
| 2 | 2_42099286_ | 42099286 | 41872146 | rs4594493 | T | C | 0.2734 | −0.0044 | 0.9956 | 0.9976 |
| | C_T | | | | | | | | | |
| 2 | 2_42137215_ | 42137215 | 41910075 | rs6734260 | A | G | 0.5073 | −0.0034 | 0.9966 | 0.9966 |
| | G_A | | | | | | | | | |
| 2 | 2_42158304_ | 42158304 | 41931164 | rs17029056 | C | A | 0.2150 | −0.0036 | 0.9964 | 0.9985 |
| | A_C | | | | | | | | | |
| 2 | 2_43505230_ | 43505230 | 43278091 | rs145471010 | T | C | 0.0209 | 0.0175 | 1.0177 | 1.0007 |
| | C_T | | | | | | | | | |
| 2 | 2_43531812_ | 43531812 | 43304673 | rs538548562 | AT | A | 0.1771 | 0.0013 | 1.0013 | 1.0005 |
| | A_AT | | | | | | | | | |
| 2 | 2_45814226_ | 45814226 | 45587087 | rs7609316 | A | T | 0.1391 | −0.0043 | 0.9957 | 0.9988 |
| | T_A | | | | | | | | | |
| 2 | 2_46065571_ | 46065571 | 45838432 | rs79942805 | G | C | 0.0401 | −0.0119 | 0.9882 | 0.9991 |
| | C_G | | | | | | | | | |
| 2 | 2_46583593_ | 46583593 | 46356454 | rs2278753 | A | G | 0.3435 | −0.0057 | 0.9943 | 0.9961 |
| | G_A | | | | | | | | | |
| 2 | 2_46595363_ | 46595363 | 46368224 | rs4145836 | A | G | 0.1153 | −0.0055 | 0.9945 | 0.9987 |
| | G_A | | | | | | | | | |
| 2 | 2_46650458_ | 46650458 | 46423319 | rs78809850 | C | T | 0.0190 | −0.0246 | 0.9757 | 0.9991 |
| | T_C | | | | | | | | | |
| 2 | 2_47248466_ | 47248466 | 47021327 | rs7565217 | G | A | 0.0937 | −0.0097 | 0.9903 | 0.9982 |
| | A_G | | | | | | | | | |
| 2 | 2_48709948_ | 48709948 | 48482809 | rs113361760 | C | T | 0.0106 | 0.0053 | 1.0053 | 1.0001 |
| | T_C | | | | | | | | | |
| 2 | 2_49141322_ | 49141322 | 48914183 | rs17037825 | C | A | 0.0506 | 0.0166 | 1.0167 | 1.0017 |
| | A_C | | | | | | | | | |
| 2 | 2_51818904_ | 51818904 | 51591766 | rs113497123 | C | T | 0.0275 | 0.0084 | 1.0084 | 1.0005 |
| | T_C | | | | | | | | | |
| 2 | 2_53039116_ | 53039116 | 52811978 | rs35688857 | A | AT | 0.1129 | −0.0153 | 0.9848 | 0.9966 |
| | AT_A | | | | | | | | | |
| 2 | 2_54830389_ | 54830389 | 54603252 | rs116707394 | G | C | 0.0265 | 0.0193 | 1.0195 | 1.0010 |
| | C_G | | | | | | | | | |
| 2 | 2_55132603_ | 55132603 | 54905466 | rs78860519 | C | T | 0.0330 | −0.0176 | 0.9826 | 0.9988 |
| | T_C | | | | | | | | | |
| 2 | 2_56787003_ | 56787003 | 56559868 | rs2194795 | A | G | 0.5982 | 0.0064 | 1.0064 | 1.0077 |
| | G_A | | | | | | | | | |
| 2 | 2_58006573_ | 58006573 | 57779438 | rs74338168 | G | A | 0.0704 | 0.0076 | 1.0076 | 1.0011 |
| | A_G | | | | | | | | | |
| 2 | 2_58455206_ | 58455206 | 58228071 | rs11461396 | ATT | AT | 0.5157 | 0.0007 | 1.0007 | 1.0007 |
| | AT_ATT | | | | | | | | | |
| 2 | 2_58844708_ | 58844708 | 58617573 | rs7582795 | T | A | 0.2366 | −0.0064 | 0.9936 | 0.9970 |
| | A_T | | | | | | | | | |
| 2 | 2_60641329_ | 60641329 | 60414194 | rs13028240 | A | G | 0.2906 | −0.0123 | 0.9878 | 0.9929 |
| | G_A | | | | | | | | | |
| 2 | 2_60669068_ | 60669068 | 60441933 | rs3028025 | ATG | A | 0.6875 | 0.0035 | 1.0035 | 1.0048 |
| | A_ATG | | | | | | | | | |
| 2 | 2_62845873_ | 62845873 | 62618738 | rs116622237 | T | G | 0.0733 | 0.0090 | 1.0090 | 1.0013 |
| | G_T | | | | | | | | | |
| 2 | 2_64894053_ | 64894053 | 64666919 | rs35123788 | TTTG | T | 0.1676 | −0.0192 | 0.9810 | 0.9936 |
| | T_TTTG | | | | | | | | | |
| 2 | 2_65618119_ | 65618119 | 65390985 | rs268128 | G | A | 0.9514 | 0.0182 | 1.0184 | 1.0353 |
| | A_G | | | | | | | | | |
| 2 | 2_65980103_ | 65980103 | 65752969 | rs139124371 | C | T | 0.0195 | 0.0100 | 1.0101 | 1.0004 |
| | T_C | | | | | | | | | |
| 2 | 2_66105510_ | 66105510 | 65878376 | rs58378948 | G | A | 0.1573 | −0.0055 | 0.9945 | 0.9983 |
| | A_G | | | | | | | | | |
| 2 | 2_67560500_ | 67560500 | 67333368 | rs148972021 | A | G | 0.0762 | −0.0004 | 0.9996 | 0.9999 |
| | G_A | | | | | | | | | |
| 2 | 2_68106897_ | 68106897 | 67879765 | rs13004783 | C | G | 0.4432 | 0.0102 | 1.0103 | 1.0091 |
| | G_C | | | | | | | | | |
| 2 | 2_68111722_ | 68111722 | 67884590 | rs7573637 | T | C | 0.4848 | 0.0007 | 1.0007 | 1.0007 |
| | C_T | | | | | | | | | |
| 2 | 2_68655836_ | 68655836 | 68428704 | rs34352314 | G | A | 0.0450 | −0.0130 | 0.9871 | 0.9988 |
| | A_G | | | | | | | | | |
| 2 | 2_68665012_ | 68665012 | 68437880 | rs11126187 | G | A | 0.8846 | 0.0035 | 1.0035 | 1.0062 |
| | A_G | | | | | | | | | |
| 2 | 2_69247526_ | 69247526 | 69020394 | rs151058084 | C | CTGT | 0.1010 | −0.0149 | 0.9852 | 0.9970 |
| | CTGTTT_C | | | | | TT | | | | |
| 2 | 2_69250632_ | 69250632 | 69023500 | rs66686620 | A | G | 0.1424 | −0.0017 | 0.9983 | 0.9995 |
| | G_A | | | | | | | | | |
| 2 | 2_69386671_ | 69386671 | 69159539 | rs5831961 | GA | G | 0.1302 | −0.0069 | 0.9931 | 0.9982 |
| | G_GA | | | | | | | | | |
| 2 | 2_69396387_ | 69396387 | 69169255 | rs10188804 | A | G | 0.4007 | 0.0044 | 1.0044 | 1.0035 |
| | G_A | | | | | | | | | |
| 2 | 2_69429294_ | 69429294 | 69202162 | rs62134902 | A | G | 0.1475 | −0.0069 | 0.9931 | 0.9980 |
| | G_A | | | | | | | | | |
| 2 | 2_69495157_ | 69495157 | 69268025 | rs62134957 | A | C | 0.0513 | −0.0230 | 0.9773 | 0.9977 |
| | C_A | | | | | | | | | |
| 2 | 2_69905400_ | 69905400 | 69678268 | rs6546540 | G | C | 0.4163 | −0.0014 | 0.9986 | 0.9988 |
| | C_G | | | | | | | | | |
| 2 | 2_69918942_ | 69918942 | 69691810 | rs12620484 | G | A | 0.5395 | −0.0063 | 0.9937 | 0.9932 |
| | A_G | | | | | | | | | |
| 2 | 2_70149873_ | 70149873 | 69922741 | rs80352135 | A | G | 0.2008 | −0.0075 | 0.9925 | 0.9970 |
| | G_A | | | | | | | | | |
| 2 | 2_70165966_ | 70165966 | 69938834 | rs7349311 | A | G | 0.1829 | −0.0034 | 0.9966 | 0.9988 |
| | G_A | | | | | | | | | |
| 2 | 2_71048778_ | 71048778 | 70821647 | rs73937022 | T | G | 0.0428 | 0.0066 | 1.0066 | 1.0006 |
| | G_T | | | | | | | | | |
| 2 | 2_72206150_ | 72206150 | 71979020 | rs114669536 | T | G | 0.0192 | 0.0149 | 1.0150 | 1.0006 |
| | G_T | | | | | | | | | |
| 2 | 2_74153422_ | 74153422 | 73926295 | rs11683738 | G | C | 0.1733 | 0.0008 | 1.0008 | 1.0003 |
| | C_G | | | | | | | | | |
| 2 | 2_76465046_ | 76465046 | 76237920 | rs12470208 | T | C | 0.5538 | −0.0058 | 0.9942 | 0.9936 |
| | C_T | | | | | | | | | |
| 2 | 2_77276201_ | 77276201 | 77049075 | rs1838878 | T | C | 0.0979 | −0.0134 | 0.9867 | 0.9974 |
| | C_T | | | | | | | | | |
| 2 | 2_85347751_ | 85347751 | 85120628 | rs75975689 | A | G | 0.1569 | 0.0008 | 1.0008 | 1.0003 |
| | G_A | | | | | | | | | |
| 2 | 2_85357265_ | 85357265 | 85130142 | rs116666195 | A | G | 0.0154 | 0.0264 | 1.0268 | 1.0008 |
| | G_A | | | | | | | | | |
| 2 | 2_85394611_ | 85394611 | 85167488 | rs11691849 | G | A | 0.3662 | −0.0106 | 0.9895 | 0.9923 |
| | A_G | | | | | | | | | |
| 2 | 2_85409239_ | 85409239 | 85182116 | rs147597376 | A | G | 0.0224 | 0.0005 | 1.0005 | 1.0000 |
| | G_A | | | | | | | | | |
| 2 | 2_86586325_ | 86586325 | 86359202 | rs58938847 | CT | C | 0.4419 | 0.0002 | 1.0002 | 1.0002 |
| | C_CT | | | | | | | | | |
| 2 | 2_86808935_ | 86808935 | 86581812 | 2:86808935_C | C | CA | 0.5093 | 0.0011 | 1.0011 | 1.0011 |
| | CA_C | | | A_C | | | | | | |
| 2 | 2_88358825_ | 88358825 | 88059306 | rs1036759 | C | G | 0.3081 | 0.0221 | 1.0223 | 1.0138 |
| | G_C | | | | | | | | | |
| 2 | 2_89045587_ | 89045587 | 88746070 | rs4972012 | T | C | 0.3180 | −0.0006 | 0.9994 | 0.9996 |
| | C_T | | | | | | | | | |
| 2 | 2_89127937_ | 89127937 | 88828424 | rs2848119 | C | A | 0.5833 | −0.0023 | 0.9977 | 0.9973 |
| | A_C | | | | | | | | | |
| 2 | 2_96640703_ | 96640703 | 95974955 | rs7563714 | C | T | 0.9520 | −0.0006 | 0.9994 | 0.9989 |
| | T_C | | | | | | | | | |
| 2 | 2_101878882_ | 101878882 | 101262420 | rs74642933 | T | C | 0.0963 | 0.0064 | 1.0064 | 1.0012 |
| | C_T | | | | | | | | | |
| 2 | 2_102279803_ | 102279803 | 101663341 | rs373462994 | CTTTATT | C | 0.5640 | 0.0000 | 1.0000 | 1.0000 |
| | C_CTTTATTTA | | | | TA | | | | | |
| 2 | 2_102334926_ | 102334926 | 101718464 | 2:102334926_ | A | AT | 0.3797 | 0.0037 | 1.0037 | 1.0028 |
| | AT_A | | | AT_A | | | | | | |
| 2 | 2_102849214_ | 102849214 | 102232754 | rs76394402 | C | T | 0.0223 | −0.0021 | 0.9979 | 0.9999 |
| | T_C | | | | | | | | | |
| 2 | 2_105307941_ | 105307941 | 104691483 | rs10178865 | T | C | 0.0275 | −0.0068 | 0.9932 | 0.9996 |
| | C_T | | | | | | | | | |
| 2 | 2_107543335_ | 107543335 | 106926879 | rs62152998 | A | G | 0.0160 | 0.0195 | 1.0197 | 1.0006 |
| | G_A | | | | | | | | | |
| 2 | 2_108642171_ | 108642171 | 108025715 | rs71419398 | C | A | 0.1336 | −0.0027 | 0.9973 | 0.9993 |
| | A_C | | | | | | | | | |
| 2 | 2_111597589_ | 111597589 | 110840012 | rs6707257 | T | C | 0.1722 | 0.0038 | 1.0038 | 1.0013 |
| | C_T | | | | | | | | | |
| 2 | 2_111629125_ | 111629125 | 110871548 | rs13029175 | T | G | 0.3148 | 0.0051 | 1.0051 | 1.0032 |
| | G_T | | | | | | | | | |
| 2 | 2_111796986_ | 111796986 | 111039409 | rs200239486 | GA | G | 0.3177 | −0.0015 | 0.9985 | 0.9990 |
| | G_GA | | | | | | | | | |
| 2 | 2_111900904_ | 111900904 | 111143327 | rs75543804 | T | G | 0.0306 | 0.0236 | 1.0239 | 1.0015 |
| | G_T | | | | | | | | | |
| 2 | 2_111919346_ | 111919346 | 111161769 | rs686952 | C | A | 0.6962 | 0.0037 | 1.0037 | 1.0052 |
| | A_C | | | | | | | | | |
| 2 | 2_111946084_ | 111946084 | 111188507 | rs532558450 | CA | C | 0.5343 | 0.0026 | 1.0026 | 1.0028 |
| | C_CA | | | | | | | | | |
| 2 | 2_111988486_ | 111988486 | 111230909 | | T | TAA | 0.2286 | −0.0049 | 0.9951 | 0.9978 |
| | TAA_T | | | | | | | | | |
| 2 | 2_111988486_ | 111988486 | 111230909 | | TA | TAA | 0.2775 | 0.0006 | 1.0006 | 1.0003 |
| | TAA_TA | | | | | | | | | |
| 2 | 2_113847915_ | 113847915 | 113090338 | rs28730394 | A | G | 0.0131 | 0.0096 | 1.0096 | 1.0003 |
| | G_A | | | | | | | | | |
| 2 | 2_115405865_ | 115405865 | 114648288 | rs74419649 | C | T | 0.1150 | −0.0034 | 0.9966 | 0.9992 |
| | T_C | | | | | | | | | |
| 2 | 2_118156200_ | 118156200 | 117398624 | rs11123419 | T | C | 0.3276 | 0.0071 | 1.0071 | 1.0047 |
| | C_T | | | | | | | | | |
| 2 | 2_118211834_ | 118211834 | 117454258 | rs34973731 | T | C | 0.1212 | 0.0087 | 1.0087 | 1.0021 |
| | C_T | | | | | | | | | |
| 2 | 2_118669853_ | 118669853 | 117912277 | rs7583655 | T | C | 0.2571 | 0.0055 | 1.0055 | 1.0028 |
| | C_T | | | | | | | | | |
| 2 | 2_118755669_ | 118755669 | 117998093 | rs17512698 | G | A | 0.5054 | −0.0033 | 0.9967 | 0.9967 |
| | A_G | | | | | | | | | |
| 2 | 2_119510491_ | 119510491 | 118752915 | | G | C | 0.9331 | 0.0039 | 1.0039 | 1.0073 |
| | C_G | | | | | | | | | |
| 2 | 2_119901805_ | 119901805 | 119144229 | rs13433007 | T | G | 0.3223 | 0.0070 | 1.0070 | 1.0045 |
| | G_T | | | | | | | | | |
| 2 | 2_121058254_ | 121058254 | 120300678 | rs6746250 | G | A | 0.7047 | −0.0118 | 0.9883 | 0.9835 |
| | A_G | | | | | | | | | |
| 2 | 2_121069922_ | 121069922 | 120312346 | rs4849857 | T | C | 0.7248 | 0.0000 | 1.0000 | 1.0000 |
| | C_T | | | | | | | | | |
| 2 | 2_121070582_ | 121070582 | 120313006 | rs11684491 | A | G | 0.5977 | 0.0000 | 1.0000 | 1.0000 |
| | G_A | | | | | | | | | |
| 2 | 2_121070811_ | 121070811 | 120313235 | rs11673874 | T | C | 0.7132 | 0.0000 | 1.0000 | 1.0000 |
| | C_T | | | | | | | | | |
| 2 | 2_121070858_ | 121070858 | 120313282 | rs745439348 | C | CG | 0.5894 | 0.0000 | 1.0000 | 1.0000 |
| | CG_C | | | | | | | | | |
| 2 | 2_121079515_ | 121079515 | 120321939 | rs4528762 | C | A | 0.2648 | 0.0000 | 1.0000 | 1.0000 |
| | A_C | | | | | | | | | |
| 2 | 2_121088182_ | 121088182 | 120330606 | rs11903787 | A | G | 0.2408 | −0.0031 | 0.9969 | 0.9985 |
| | G_A | | | | | | | | | |
| 2 | 2_121089731_ | 121089731 | 120332155 | rs17625845 | C | T | 0.1943 | −0.0162 | 0.9839 | 0.9938 |
| | T_C | | | | | | | | | |
| 2 | 2_121095920_ | 121095920 | 120338344 | rs7589138 | C | T | 0.4180 | 0.0000 | 1.0000 | 1.0000 |
| | T_C | | | | | | | | | |
| 2 | 2_121125114_ | 121125114 | 120367538 | rs10198584 | C | G | 0.6077 | 0.0042 | 1.0042 | 1.0051 |
| | G_C | | | | | | | | | |
| 2 | 2_121125289_ | 121125289 | 120367713 | rs1992131 | T | C | 0.1188 | 0.0010 | 1.0010 | 1.0002 |
| | C_T | | | | | | | | | |
| 2 | 2_121126279_ | 121126279 | 120368703 | rs12622286 | A | G | 0.6601 | 0.0029 | 1.0029 | 1.0038 |
| | G_A | | | | | | | | | |
| 2 | 2_121159205_ | 121159205 | 120401629 | rs10164550 | A | G | 0.3527 | −0.0147 | 0.9854 | 0.9897 |
| | G_A | | | | | | | | | |
| 2 | 2_121160898_ | 121160898 | 120403322 | rs76476871 | T | C | 0.0873 | 0.0117 | 1.0118 | 1.0021 |
| | C_T | | | | | | | | | |
| 2 | 2_121238571_ | 121238571 | 120480995 | 2:121238571 | AGT | AGTG | 0.8845 | 0.0077 | 1.0077 | 1.0137 |
| | AGTGT_AGT | | | AGT_A | | T | | | | |
| 2 | 2_121246568_ | 121246568 | 120488992 | rs10179592 | C | T | 0.8970 | 0.0563 | 1.0579 | 1.1066 |
| | T_C | | | | | | | | | |
| 2 | 2_121852657_ | 121852657 | 121095081 | rs13409556 | T | C | 0.0263 | −0.0056 | 0.9944 | 0.9997 |
| | C_T | | | | | | | | | |
| 2 | 2_121854954_ | 121854954 | 121097378 | rs28374086 | G | C | 0.0189 | −0.0043 | 0.9957 | 0.9998 |
| | C_G | | | | | | | | | |
| 2 | 2_122966476_ | 122966476 | 122208900 | rs56080854 | T | C | 0.1588 | 0.0037 | 1.0037 | 1.0012 |
| | C_T | | | | | | | | | |
| 2 | 2_123784114_ | 123784114 | 123026538 | rs115470088 | G | A | 0.0197 | 0.0000 | 1.0000 | 1.0000 |
| | A_G | | | | | | | | | |
| 2 | 2_124310905_ | 124310905 | 123553329 | rs13398935 | T | C | 0.0745 | 0.0094 | 1.0094 | 1.0014 |
| | C_T | | | | | | | | | |
| 2 | 2_124766981_ | 124766981 | 124009404 | rs12478765 | A | G | 0.2546 | 0.0083 | 1.0083 | 1.0042 |
| | G_A | | | | | | | | | |
| 2 | 2_128302536_ | 128302536 | 127544961 | rs148135359 | G | A | 0.1450 | −0.0070 | 0.9930 | 0.9980 |
| | A_G | | | | | | | | | |
| 2 | 2_129915194_ | 129915194 | 129157621 | rs775250244 | G | GTTG | 0.6350 | −0.0120 | 0.9881 | 0.9849 |
| | GTTGAC_G | | | | | AC | | | | |
| 2 | 2_133952040_ | 133952040 | 133194468 | rs12691834 | A | G | 0.2580 | −0.0030 | 0.9970 | 0.9985 |
| | G_A | | | | | | | | | |
| 2 | 2_134356301_ | 134356301 | 133598730 | rs12987374 | T | C | 0.5358 | 0.0019 | 1.0019 | 1.0020 |
| | C_T | | | | | | | | | |
| 2 | 2_135158254_ | 135158254 | 134400683 | rs13007251 | G | A | 0.2286 | −0.0094 | 0.9906 | 0.9957 |
| | A_G | | | | | | | | | |
| 2 | 2_135284038_ | 135284038 | 134526467 | rs479980 | A | C | 0.5864 | −0.0032 | 0.9968 | 0.9963 |
| | C_A | | | | | | | | | |
| 2 | 2_145205693_ | 145205693 | 144448126 | rs10187892 | C | G | 0.5371 | 0.0063 | 1.0063 | 1.0068 |
| | G_C | | | | | | | | | |
| 2 | 2_145976169_ | 145976169 | 145218601 | rs59116697 | T | A | 0.4184 | 0.0047 | 1.0047 | 1.0039 |
| | A_T | | | | | | | | | |
| 2 | 2_146008395_ | 146008395 | 145250827 | rs767604124 | A | AG | 0.2291 | −0.0103 | 0.9898 | 0.9953 |
| | AG_A | | | | | | | | | |
| 2 | 2_153266604_ | 153266604 | 152410090 | rs73967988 | T | C | 0.0131 | 0.0059 | 1.0059 | 1.0002 |
| | C_T | | | | | | | | | |
| 2 | 2_160750861_ | 160750861 | 159894350 | rs1511224 | C | T | 0.4127 | 0.0013 | 1.0013 | 1.0011 |
| | T_C | | | | | | | | | |
| 2 | 2_160781240_ | 160781240 | 159924729 | rs796929846 | T | TG | 0.3882 | 0.0047 | 1.0047 | 1.0037 |
| | TG_T | | | | | | | | | |
| 2 | 2_166297783_ | 166297783 | 165441273 | rs190067614 | A | G | 0.0172 | 0.0104 | 1.0105 | 1.0004 |
| | G_A | | | | | | | | | |
| 2 | 2_168581285_ | 168581285 | 167724775 | rs67678493 | G | A | 0.2472 | 0.0105 | 1.0106 | 1.0052 |
| | A_G | | | | | | | | | |
| 2 | 2_168611545_ | 168611545 | 167755035 | rs115102047 | G | C | 0.0448 | 0.0119 | 1.0120 | 1.0011 |
| | C_G | | | | | | | | | |
| 2 | 2_171504324_ | 171504324 | 170647814 | rs12151651 | T | C | 0.1366 | 0.0039 | 1.0039 | 1.0011 |
| | C_T | | | | | | | | | |
| 2 | 2_172158381_ | 172158381 | 171301871 | rs72874644 | G | C | 0.1048 | 0.0028 | 1.0028 | 1.0006 |
| | C_G | | | | | | | | | |
| 2 | 2_172369400_ | 172369400 | 171512890 | rs10490603 | C | T | 0.1379 | −0.0057 | 0.9943 | 0.9984 |
| | T_C | | | | | | | | | |
| 2 | 2_172380723_ | 172380723 | 171524213 | rs11681001 | C | G | 0.3728 | 0.0000 | 1.0000 | 1.0000 |
| | G_C | | | | | | | | | |
| 2 | 2_172382596_ | 172382596 | 171526086 | rs112352153 | A | G | 0.1221 | −0.0037 | 0.9963 | 0.9991 |
| | G_A | | | | | | | | | |
| 2 | 2_172424963_ | 172424963 | 171568453 | rs2542943 | T | C | 0.5467 | −0.0046 | 0.9954 | 0.9950 |
| | C_T | | | | | | | | | |
| 2 | 2_172425405_ | 172425405 | 171568895 | rs765335847 | A | AAAG | 0.2350 | 0.0041 | 1.0041 | 1.0019 |
| | AAAGT_A | | | | | T | | | | |
| 2 | 2_172428729_ | 172428729 | 171572219 | rs2356791 | A | G | 0.6398 | 0.0000 | 1.0000 | 1.0000 |
| | G_A | | | | | | | | | |
| 2 | 2_172972828_ | 172972828 | 172108100 | rs2357322 | C | G | 0.3460 | 0.0000 | 1.0000 | 1.0000 |
| | G_C | | | | | | | | | |
| 2 | 2_172974566_ | 172974566 | 172109838 | rs17726078 | G | C | 0.4743 | −0.0258 | 0.9745 | 0.9760 |
| | C_G | | | | | | | | | |
| 2 | 2_173027109_ | 173027109 | 172162381 | rs79238150 | T | C | 0.0175 | 0.0131 | 1.0132 | 1.0005 |
| | C_T | | | | | | | | | |
| 2 | 2_173041097_ | 173041097 | 172176369 | rs13412383 | C | T | 0.1415 | 0.0017 | 1.0017 | 1.0005 |
| | T_C | | | | | | | | | |
| 2 | 2_173296850_ | 173296850 | 172432122 | rs36055280 | A | G | 0.3366 | 0.0020 | 1.0020 | 1.0013 |
| | G_A | | | | | | | | | |
| 2 | 2_173424800_ | 173424800 | 172560072 | rs6723492 | T | G | 0.2097 | −0.0042 | 0.9958 | 0.9982 |
| | G_T | | | | | | | | | |
| 2 | 2_174104508_ | 174104508 | 173239780 | rs62174362 | G | A | 0.0375 | 0.0036 | 1.0036 | 1.0003 |
| | A_G | | | | | | | | | |
| 2 | 2_174204524_ | 174204524 | 173339796 | rs7604661 | C | G | 0.8562 | 0.0000 | 1.0000 | 1.0000 |
| | G_C | | | | | | | | | |
| 2 | 2_174212910_ | 174212910 | 173348182 | rs1550622 | G | A | 0.8450 | 0.0222 | 1.0224 | 1.0383 |
| | A_G | | | | | | | | | |
| 2 | 2_174242984_ | 174242984 | 173378256 | rs144869769 | T | G | 0.0308 | 0.0049 | 1.0049 | 1.0003 |
| | G_T | | | | | | | | | |
| 2 | 2_174276092_ | 174276092 | 173411364 | rs7571484 | C | T | 0.2008 | 0.0042 | 1.0042 | 1.0017 |
| | T_C | | | | | | | | | |
| 2 | 2_174584779_ | 174584779 | 173720051 | rs1371439 | G | C | 0.0260 | 0.0148 | 1.0149 | 1.0008 |
| | C_G | | | | | | | | | |
| 2 | 2_174705699_ | 174705699 | 173840971 | rs75627816 | G | A | 0.0130 | 0.0211 | 1.0213 | 1.0006 |
| | A_G | | | | | | | | | |
| 2 | 2_177375311_ | 177375311 | 176510583 | 2:177375311_ | C | CAG | 0.1223 | 0.0000 | 1.0000 | 1.0000 |
| | CAG_C | | | CAG_C | | | | | | |
| 2 | 2_178877989_ | 178877989 | 178013262 | rs17636214 | A | G | 0.0399 | 0.0105 | 1.0106 | 1.0008 |
| | G_A | | | | | | | | | |
| 2 | 2_180145058_ | 180145058 | 179280331 | rs77076967 | G | C | 0.0552 | 0.0058 | 1.0058 | 1.0006 |
| | C_G | | | | | | | | | |
| 2 | 2_180438264_ | 180438264 | 179573537 | rs2222032 | A | T | 0.0898 | 0.0021 | 1.0021 | 1.0004 |
| | T_A | | | | | | | | | |
| 2 | 2_180448523_ | 180448523 | 179583796 | rs11695089 | G | A | 0.0868 | 0.0026 | 1.0026 | 1.0005 |
| | A_G | | | | | | | | | |
| 2 | 2_180934312_ | 180934312 | 180069585 | rs71425659 | T | A | 0.0454 | 0.0200 | 1.0202 | 1.0018 |
| | A_T | | | | | | | | | |
| 2 | 2_181000793_ | 181000793 | 180136066 | rs10175272 | C | T | 0.2159 | 0.0106 | 1.0107 | 1.0046 |
| | T_C | | | | | | | | | |
| 2 | 2_181659051_ | 181659051 | 180794324 | rs11686097 | G | A | 0.0759 | −0.0052 | 0.9948 | 0.9992 |
| | A_G | | | | | | | | | |
| 2 | 2_185343431_ | 185343431 | 184478704 | rs72893750 | A | G | 0.0155 | −0.0030 | 0.9970 | 0.9999 |
| | G_A | | | | | | | | | |
| 2 | 2_186761497_ | 186761497 | 185896770 | rs59190502 | T | C | 0.2421 | −0.0067 | 0.9933 | 0.9968 |
| | C_T | | | | | | | | | |
| 2 | 2_187757692_ | 187757692 | 186892965 | rs10931260 | C | T | 0.2638 | −0.0012 | 0.9988 | 0.9994 |
| | T_C | | | | | | | | | |
| 2 | 2_187782375_ | 187782375 | 186917648 | rs1901341 | G | T | 0.4504 | 0.0037 | 1.0037 | 1.0033 |
| | T_G | | | | | | | | | |
| 2 | 2_188871632_ | 188871632 | 188006905 | 2:188871632_ | C | CCTT | 0.1269 | 0.0053 | 1.0053 | 1.0013 |
| | CCTTT_C | | | CCTTT_C | | T | | | | |
| 2 | 2_188872284_ | 188872284 | 188007557 | rs3060834 | TACAC | T | 0.7770 | −0.0075 | 0.9925 | 0.9884 |
| | T_TACACACAC | | | | ACAC | | | | | |
| 2 | 2_189684112_ | 189684112 | 188819386 | rs529161674 | C | A | 0.0286 | −0.0150 | 0.9851 | 0.9991 |
| | A_C | | | | | | | | | |
| 2 | 2_189805077_ | 189805077 | 188940351 | rs7588568 | A | C | 0.2810 | −0.0036 | 0.9964 | 0.9980 |
| | C_A | | | | | | | | | |
| 2 | 2_189882065_ | 189882065 | 189017339 | rs10175784 | G | T | 0.4470 | −0.0021 | 0.9979 | 0.9981 |
| | T_G | | | | | | | | | |
| 2 | 2_190037694_ | 190037694 | 189172968 | rs762595062 | C | CT | 0.6987 | 0.0080 | 1.0080 | 1.0113 |
| | CT_C | | | | | | | | | |
| 2 | 2_190305048_ | 190305048 | 189440322 | rs75116627 | T | A | 0.0121 | −0.0102 | 0.9899 | 0.9998 |
| | A_T | | | | | | | | | |
| 2 | 2_190410532_ | 190410532 | 189545806 | rs74637811 | T | C | 0.0117 | −0.0109 | 0.9892 | 0.9997 |
| | C_T | | | | | | | | | |
| 2 | 2_191333980_ | 191333980 | 190469254 | rs147470027 | CT | C | 0.0188 | 0.0000 | 1.0000 | 1.0000 |
| | C_CT | | | | | | | | | |
| 2 | 2_192066082_ | 192066082 | 191201356 | rs17510839 | A | G | 0.0414 | −0.0037 | 0.9963 | 0.9997 |
| | G_A | | | | | | | | | |
| 2 | 2_192381934_ | 192381934 | 191517208 | rs2356656 | T | C | 0.8588 | 0.0000 | 1.0000 | 1.0000 |
| | C_T | | | | | | | | | |
| 2 | 2_194256253_ | 194256253 | 193391528 | rs147035132 | C | T | 0.0144 | −0.0247 | 0.9756 | 0.9993 |
| | T_C | | | | | | | | | |
| 2 | 2_195931292_ | 195931292 | 195066568 | rs1991632 | A | G | 0.0516 | 0.0091 | 1.0091 | 1.0009 |
| | G_A | | | | | | | | | |
| 2 | 2_195954734_ | 195954734 | 195090010 | rs777717 | G | A | 0.9244 | −0.0107 | 0.9894 | 0.9804 |
| | A_G | | | | | | | | | |
| 2 | 2_200004974_ | 200004974 | 199140251 | rs4673248 | T | G | 0.5721 | 0.0098 | 1.0098 | 1.0113 |
| | G_T | | | | | | | | | |
| 2 | 2_201559027_ | 201559027 | 200694304 | rs116056056 | G | A | 0.0393 | −0.0131 | 0.9870 | 0.9990 |
| | A_G | | | | | | | | | |
| 2 | 2_201559931_ | 201559931 | 200695208 | rs868396 | A | G | 0.1268 | 0.0034 | 1.0034 | 1.0009 |
| | G_A | | | | | | | | | |
| 2 | 2_201912518_ | 201912518 | 201047795 | rs758917394 | A | ACTG | 0.0686 | −0.0062 | 0.9938 | 0.9992 |
| | ACTGTTAAAGG | | | | | TTAA | | | | |
| | GC_A | | | | | AGGG | | | | |
| | | | | | | C | | | | |
| 2 | 2_201962858_ | 201962858 | 201098135 | rs145395636 | T | A | 0.0211 | 0.0131 | 1.0132 | 1.0006 |
| | A_T | | | | | | | | | |
| 2 | 2_201990540_ | 201990540 | 201125817 | rs13015648 | G | T | 0.0638 | 0.0195 | 1.0197 | 1.0025 |
| | T_G | | | | | | | | | |
| 2 | 2_202004676_ | 202004676 | 201139953 | rs6759004 | T | C | 0.1525 | −0.0004 | 0.9996 | 0.9999 |
| | C_T | | | | | | | | | |
| 2 | 2_202096254_ | 202096254 | 201231531 | rs17860411 | T | C | 0.0139 | 0.0090 | 1.0090 | 1.0003 |
| | C_T | | | | | | | | | |
| 2 | 2_202123430_ | 202123430 | 201258707 | rs3769821 | T | C | 0.6670 | −0.0067 | 0.9933 | 0.9911 |
| | C_T | | | | | | | | | |
| 2 | 2_202170655_ | 202170655 | 201305932 | rs10200279 | C | T | 0.7139 | −0.0050 | 0.9950 | 0.9929 |
| | T_C | | | | | | | | | |
| 2 | 2_202198589_ | 202198589 | 201333866 | rs566570823 | T | C | 0.0261 | 0.0116 | 1.0117 | 1.0006 |
| | C_T | | | | | | | | | |
| 2 | 2_202204741_ | 202204741 | 201340018 | rs10197246 | C | T | 0.7210 | −0.0149 | 0.9852 | 0.9788 |
| | T_C | | | | | | | | | |
| 2 | 2_202242307_ | 202242307 | 201377584 | rs7616 | G | C | 0.0674 | −0.0069 | 0.9931 | 0.9991 |
| | C_G | | | | | | | | | |
| 2 | 2_202379828_ | 202379828 | 201515105 | rs59278883 | A | T | 0.0763 | −0.0133 | 0.9868 | 0.9980 |
| | T_A | | | | | | | | | |
| 2 | 2_202395302_ | 202395302 | 201530579 | 2:202395302_ | T | TA | 0.7456 | −0.0014 | 0.9986 | 0.9979 |
| | TA_T | | | TA_T | | | | | | |
| 2 | 2_202531264_ | 202531264 | 201666541 | rs62193398 | C | T | 0.4174 | 0.0119 | 1.0120 | 1.0100 |
| | T_C | | | | | | | | | |
| 2 | 2_205638110_ | 205638110 | 204773387 | rs7599107 | C | CAT | 0.0337 | 0.0062 | 1.0062 | 1.0004 |
| | CAT_C | | | | | | | | | |
| 2 | 2_205860163_ | 205860163 | 204995440 | rs199556273 | T | C | 0.4595 | −0.0021 | 0.9979 | 0.9981 |
| | C_T | | | | | | | | | |
| 2 | 2_205901702_ | 205901702 | 205036979 | 2:205901702_ | T | TA | 0.1110 | 0.0148 | 1.0149 | 1.0033 |
| | TA_T | | | TA_T | | | | | | |
| 2 | 2_205922601_ | 205922601 | 205057878 | rs35436581 | TA | T | 0.3386 | −0.0100 | 0.9900 | 0.9933 |
| | T_TA | | | | | | | | | |
| 2 | 2_205991363_ | 205991363 | 205126639 | rs112373916 | A | G | 0.0783 | −0.0014 | 0.9986 | 0.9998 |
| | G_A | | | | | | | | | |
| 2 | 2_207521558_ | 207521558 | 206656834 | rs6749661 | C | T | 0.1822 | 0.0044 | 1.0044 | 1.0016 |
| | T_C | | | | | | | | | |
| 2 | 2_208087325_ | 208087325 | 207222601 | rs10203899 | C | G | 0.0566 | −0.0075 | 0.9925 | 0.9992 |
| | G_C | | | | | | | | | |
| 2 | 2_208977402_ | 208977402 | 208112678 | rs55999272 | G | A | 0.3754 | −0.0057 | 0.9943 | 0.9957 |
| | A_G | | | | | | | | | |
| 2 | 2_211620445_ | 211620445 | 210755721 | rs2193619 | C | T | 0.1325 | −0.0050 | 0.9950 | 0.9987 |
| | T_C | | | | | | | | | |
| 2 | 2_213414265_ | 213414265 | 212549541 | rs13427822 | G | A | 0.2724 | 0.0066 | 1.0066 | 1.0036 |
| | A_G | | | | | | | | | |
| 2 | 2_214087853_ | 214087853 | 213223129 | rs62186589 | A | G | 0.2095 | −0.0036 | 0.9964 | 0.9985 |
| | G_A | | | | | | | | | |
| 2 | 2_215928972_ | 215928972 | 215064249 | rs1523721 | C | G | 0.5767 | 0.0007 | 1.0007 | 1.0008 |
| | G_C | | | | | | | | | |
| 2 | 2_217001090_ | 217001090 | 216136367 | rs564652267 | AAAAA | A | 0.7472 | −0.0094 | 0.9906 | 0.9861 |
| | A_AAAAAAG | | | | AG | | | | | |
| 2 | 2_217004038_ | 217004038 | 216139315 | rs759482711 | T | TAC | 0.4694 | −0.0015 | 0.9985 | 0.9986 |
| | TAC_T | | | | | | | | | |
| 2 | 2_217030284_ | 217030284 | 216165561 | rs16855522 | G | A | 0.1326 | 0.0083 | 1.0083 | 1.0022 |
| | A_G | | | | | | | | | |
| 2 | 2_217290060_ | 217290060 | 216425337 | rs2738280 | T | C | 0.0388 | −0.0200 | 0.9802 | 0.9985 |
| | C_T | | | | | | | | | |
| 2 | 2_217582016_ | 217582016 | 216717293 | rs9646854 | T | C | 0.0616 | −0.0018 | 0.9982 | 0.9998 |
| | C_T | | | | | | | | | |
| 2 | 2_217858641_ | 217858641 | 216993918 | rs76546018 | A | G | 0.0308 | −0.0032 | 0.9968 | 0.9998 |
| | G_A | | | | | | | | | |
| 2 | 2_217859657_ | 217859657 | 216994934 | rs10191184 | A | G | 0.1268 | −0.0078 | 0.9922 | 0.9980 |
| | G_A | | | | | | | | | |
| 2 | 2_217894234_ | 217894234 | 217029511 | rs12465506 | A | G | 0.4956 | −0.0013 | 0.9987 | 0.9987 |
| | G_A | | | | | | | | | |
| 2 | 2_217912437_ | 217912437 | 217047714 | rs77549302 | T | C | 0.0522 | 0.0000 | 1.0000 | 1.0000 |
| | C_T | | | | | | | | | |
| 2 | 2_217914253_ | 217914253 | 217049530 | rs10178467 | G | A | 0.0302 | 0.0000 | 1.0000 | 1.0000 |
| | A_G | | | | | | | | | |
| 2 | 2_217920769_ | 217920769 | 217056046 | rs4442975 | T | G | 0.5001 | −0.1078 | 0.8978 | 0.9004 |
| | G_T | | | | | | | | | |
| 2 | 2_217936015_ | 217936015 | 217071292 | rs12622536 | C | T | 0.4074 | 0.0000 | 1.0000 | 1.0000 |
| | T_C | | | | | | | | | |
| 2 | 2_217955896_ | 217955896 | 217091173 | 2:217955896_ | G | GA | 0.0382 | −0.1086 | 0.8971 | 0.9922 |
| | GA_G | | | GA_G | | | | | | |
| 2 | 2_217963060_ | 217963060 | 217098337 | rs34005590 | A | C | 0.0499 | −0.0277 | 0.9727 | 0.9973 |
| | C_A | | | | | | | | | |
| 2 | 2_217965655_ | 217965655 | 217100932 | rs2372964 | A | T | 0.7946 | 0.0000 | 1.0000 | 1.0000 |
| | T_A | | | | | | | | | |
| 2 | 2_217965676_ | 217965676 | 217100953 | rs78464992 | A | T | 0.0532 | −0.0059 | 0.9941 | 0.9994 |
| | T_A | | | | | | | | | |
| 2 | 2_217967717_ | 217967717 | 217102994 | rs2372965 | A | C | 0.7609 | 0.0000 | 1.0000 | 1.0000 |
| | C_A | | | | | | | | | |
| 2 | 2_218148853_ | 218148853 | 217284130 | rs148721366 | T | C | 0.0198 | 0.0108 | 1.0109 | 1.0004 |
| | C_T | | | | | | | | | |
| 2 | 2_218159244_ | 218159244 | 217294521 | rs6752033 | C | T | 0.8291 | 0.0009 | 1.0009 | 1.0015 |
| | T_C | | | | | | | | | |
| 2 | 2_218174384_ | 218174384 | 217309661 | rs758533999 | T | TGA | 0.1402 | −0.0003 | 0.9997 | 0.9999 |
| | TGA_T | | | | | | | | | |
| 2 | 2_218181721_ | 218181721 | 217316998 | rs79286679 | C | T | 0.1243 | −0.0016 | 0.9984 | 0.9996 |
| | T_C | | | | | | | | | |
| 2 | 2_218219226_ | 218219226 | 217354503 | rs55848226 | A | G | 0.1058 | −0.0127 | 0.9874 | 0.9973 |
| | G_A | | | | | | | | | |
| 2 | 2_218262547_ | 218262547 | 217397824 | rs899278 | A | G | 0.0243 | 0.0367 | 1.0374 | 1.0018 |
| | G_A | | | | | | | | | |
| 2 | 2_218292158_ | 218292158 | 217427435 | rs11693806 | G | C | 0.7309 | −0.0341 | 0.9665 | 0.9516 |
| | C_G | | | | | | | | | |
| 2 | 2_218303958_ | 218303958 | 217439235 | rs2568176 | A | G | 0.2905 | 0.0189 | 1.0191 | 1.0111 |
| | G_A | | | | | | | | | |
| 2 | 2_218677711_ | 218677711 | 217812988 | rs78297591 | A | G | 0.1009 | −0.0023 | 0.9977 | 0.9995 |
| | G_A | | | | | | | | | |
| 2 | 2_218714845_ | 218714845 | 217850122 | rs3791977 | A | G | 0.3917 | −0.0144 | 0.9857 | 0.9888 |
| | G_A | | | | | | | | | |
| 2 | 2_218722506_ | 218722506 | 217857783 | rs3791959 | G | C | 0.3298 | 0.0061 | 1.0061 | 1.0040 |
| | C_G | | | | | | | | | |
| 2 | 2_218723455_ | 218723455 | 217858732 | rs6436017 | G | A | 0.5098 | 0.0010 | 1.0010 | 1.0010 |
| | A_G | | | | | | | | | |
| 2 | 2_219963550_ | 219963550 | 219098828 | rs11692085 | T | C | 0.6393 | −0.0011 | 0.9989 | 0.9986 |
| | C_T | | | | | | | | | |
| 2 | 2_222865746_ | 222865746 | 222001027 | rs1030280 | C | T | 0.7345 | 0.0017 | 1.0017 | 1.0025 |
| | T_C | | | | | | | | | |
| 2 | 2_222904550_ | 222904550 | 222039831 | rs775726741 | A | AC | 0.1549 | −0.0024 | 0.9976 | 0.9993 |
| | AC_A | | | | | | | | | |
| 2 | 2_227226952_ | 227226952 | 226362236 | rs12479355 | G | A | 0.2105 | −0.0010 | 0.9990 | 0.9996 |
| | A_G | | | | | | | | | |
| 2 | 2_227228251_ | 227228251 | 226363535 | rs76367336 | C | T | 0.1191 | −0.0020 | 0.9980 | 0.9995 |
| | T_C | | | | | | | | | |
| 2 | 2_227254695_ | 227254695 | 226389979 | rs13020901 | G | A | 0.3354 | 0.0053 | 1.0053 | 1.0036 |
| | A_G | | | | | | | | | |
| 2 | 2_227274762_ | 227274762 | 226410046 | rs2396351 | G | C | 0.6559 | 0.0054 | 1.0054 | 1.0071 |
| | C_G | | | | | | | | | |
| 2 | 2_227344207_ | 227344207 | 226479491 | rs13029395 | T | C | 0.1710 | 0.0084 | 1.0084 | 1.0029 |
| | C_T | | | | | | | | | |
| 2 | 2_227346475_ | 227346475 | 226481759 | rs192188884 | A | G | 0.1275 | −0.0041 | 0.9959 | 0.9990 |
| | G_A | | | | | | | | | |
| 2 | 2_227358407_ | 227358407 | 226493691 | rs148731541 | G | A | 0.0311 | −0.0148 | 0.9853 | 0.9991 |
| | A_G | | | | | | | | | |
| 2 | 2_233520942_ | 233520942 | 232656232 | rs12694911 | C | T | 0.5332 | 0.0058 | 1.0058 | 1.0062 |
| | T_C | | | | | | | | | |
| 2 | 2_236025273_ | 236025273 | 235116629 | rs13398186 | C | G | 0.2216 | −0.0068 | 0.9932 | 0.9970 |
| | G_C | | | | | | | | | |
| 2 | 2_238277795_ | 238277795 | 237369152 | rs2646260 | G | A | 0.2355 | −0.0042 | 0.9958 | 0.9980 |
| | A_G | | | | | | | | | |
| 2 | 2_238299230_ | 238299230 | 237390587 | rs12052971 | A | G | 0.2542 | −0.0008 | 0.9992 | 0.9996 |
| | G_A | | | | | | | | | |
| 2 | 2_238508099_ | 238508099 | 237599456 | rs1316476 | C | T | 0.2817 | 0.0023 | 1.0023 | 1.0013 |
| | T_C | | | | | | | | | |
| 2 | 2_238509759_ | 238509759 | 237601116 | rs11894280 | A | G | 0.0788 | 0.0005 | 1.0005 | 1.0001 |
| | G_A | | | | | | | | | |
| 2 | 2_238511029_ | 238511029 | 237602386 | rs4663260 | C | T | 0.0660 | 0.0011 | 1.0011 | 1.0001 |
| | T_C | | | | | | | | | |
| 2 | 2_238581009_ | 238581009 | 237672366 | rs7562075 | C | T | 0.6830 | −0.0089 | 0.9911 | 0.9879 |
| | T_C | | | | | | | | | |
| 2 | 2_239479931_ | 239479931 | 238571290 | rs11690762 | A | G | 0.3851 | 0.0011 | 1.0011 | 1.0008 |
| | G_A | | | | | | | | | |
| 2 | 2_239506956_ | 239506956 | 238598315 | rs143887241 | G | A | 0.0119 | −0.0295 | 0.9709 | 0.9993 |
| | A_G | | | | | | | | | |
| 2 | 2_240987052_ | 240987052 | 240047635 | rs79166293 | C | A | 0.0397 | −0.0130 | 0.9871 | 0.9990 |
| | A_C | | | | | | | | | |
| 2 | 2_241313213_ | 241313213 | 240373796 | rs34352794 | T | A | 0.2720 | −0.0011 | 0.9989 | 0.9994 |
| | A_T | | | | | | | | | |
| 2 | 2_241341294_ | 241341294 | 240401877 | rs11271854 | GGGAGA | G | 0.2739 | −0.0059 | 0.9941 | 0.9968 |
| | G_GGGAGAAGAA | | | | AGAA | | | | | |
| 2 | 2_241344383_ | 241344383 | 240404966 | rs10195442 | G | C | 0.1856 | −0.0016 | 0.9984 | 0.9994 |
| | C_G | | | | | | | | | |
| 2 | 2_241388857_ | 241388857 | 240449440 | rs4676356 | A | C | 0.9772 | −0.0686 | 0.9337 | 0.8746 |
| | C_A | | | | | | | | | |
| 3 | 3_118118_ | 118118 | 76435 | rs12633924 | G | C | 0.1908 | 0.0000 | 1.0000 | 1.0000 |
| | C_G | | | | | | | | | |
| 3 | 3_253679_ | 253679 | 211996 | rs17329688 | T | C | 0.1394 | −0.0051 | 0.9949 | 0.9986 |
| | C_T | | | | | | | | | |
| 3 | 3_1882270_ | 1882270 | 1840586 | rs6807710 | T | C | 0.5197 | 0.0044 | 1.0044 | 1.0046 |
| | C_T | | | | | | | | | |
| 3 | 3_4733966_ | 4733966 | 4692282 | rs34151611 | CA | C | 0.5524 | 0.0045 | 1.0045 | 1.0050 |
| | C_CA | | | | | | | | | |
| 3 | 3_4742251_ | 4742251 | 4700567 | rs6762558 | G | A | 0.3802 | 0.0284 | 1.0288 | 1.0220 |
| | A_G | | | | | | | | | |
| 3 | 3_4764437_ | 4764437 | 4722753 | rs55864359 | C | T | 0.4512 | 0.0013 | 1.0013 | 1.0012 |
| | T_C | | | | | | | | | |
| 3 | 3_4869622_ | 4869622 | 4827938 | rs3805026 | C | T | 0.1911 | 0.0190 | 1.0192 | 1.0073 |
| | T_C | | | | | | | | | |
| 3 | 3_6885152_ | 6885152 | 6843465 | rs150612085 | C | G | 0.0138 | 0.0000 | 1.0000 | 1.0000 |
| | G_C | | | | | | | | | |
| 3 | 3_8557875_ | 8557875 | 8516189 | rs6802843 | A | T | 0.5070 | 0.0000 | 1.0000 | 1.0000 |
| | T_A | | | | | | | | | |
| 3 | 3_8615277_ | 8615277 | 8573591 | rs164966 | A | G | 0.7860 | 0.0082 | 1.0082 | 1.0130 |
| | G_A | | | | | | | | | |
| 3 | 3_9318416_ | 9318416 | 9276732 | rs17050210 | G | T | 0.6254 | 0.0105 | 1.0106 | 1.0132 |
| | T_G | | | | | | | | | |
| 3 | 3_10258192_ | 10258192 | 10216508 | rs696322 | C | G | 0.0744 | 0.0000 | 1.0000 | 1.0000 |
| | G_C | | | | | | | | | |
| 3 | 3_10261294_ | 10261294 | 10219610 | rs3895947 | G | A | 0.7695 | 0.0000 | 1.0000 | 1.0000 |
| | A_G | | | | | | | | | |
| 3 | 3_10310878_ | 10310878 | 10269194 | rs2270455 | G | T | 0.1009 | −0.0017 | 0.9983 | 0.9997 |
| | T_G | | | | | | | | | |
| 3 | 3_10326169_ | 10326169 | 10284485 | rs171407 | A | G | 0.5327 | −0.0098 | 0.9902 | 0.9896 |
| | G_A | | | | | | | | | |
| 3 | 3_11050452_ | 11050452 | 11008766 | rs2950800 | C | T | 0.7306 | 0.0033 | 1.0033 | 1.0048 |
| | T_C | | | | | | | | | |
| 3 | 3_11055624_ | 11055624 | 11013938 | rs2933308 | T | C | 0.3922 | −0.0028 | 0.9972 | 0.9978 |
| | C_T | | | | | | | | | |
| 3 | 3_11059996_ | 11059996 | 11018310 | rs1728803 | G | A | 0.6018 | 0.0026 | 1.0026 | 1.0031 |
| | A_G | | | | | | | | | |
| 3 | 3_12329452_ | 12329452 | 12287953 | rs17029006 | T | C | 0.2779 | 0.0124 | 1.0125 | 1.0069 |
| | C_T | | | | | | | | | |
| 3 | 3_12508167_ | 12508167 | 12466668 | rs6793530 | T | C | 0.3351 | 0.0138 | 1.0139 | 1.0093 |
| | C_T | | | | | | | | | |
| 3 | 3_13611731_ | 13611731 | 13570231 | rs3832210 | GC | G | 0.0564 | 0.0232 | 1.0235 | 1.0026 |
| | G_GC | | | | | | | | | |
| 3 | 3_14137393_ | 14137393 | 14095893 | rs77027159 | G | A | 0.0213 | 0.0350 | 1.0356 | 1.0015 |
| | A_G | | | | | | | | | |
| 3 | 3_14347085_ | 14347085 | 14305585 | rs9837707 | C | T | 0.9547 | −0.0105 | 0.9896 | 0.9802 |
| | T_C | | | | | | | | | |
| 3 | 3_15384049_ | 15384049 | 15342542 | rs74716230 | G | A | 0.0340 | −0.0062 | 0.9938 | 0.9996 |
| | A_G | | | | | | | | | |
| 3 | 3_16788466_ | 16788466 | 16746959 | rs13067457 | G | A | 0.6642 | 0.0105 | 1.0106 | 1.0141 |
| | A_G | | | | | | | | | |
| 3 | 3_18129387_ | 18129387 | 18087895 | rs115551171 | G | C | 0.0182 | −0.0101 | 0.9900 | 0.9996 |
| | C_G | | | | | | | | | |
| 3 | 3_18463734_ | 18463734 | 18422242 | rs141634179 | C | G | 0.0239 | −0.0269 | 0.9735 | 0.9987 |
| | G_C | | | | | | | | | |
| 3 | 3_22410483_ | 22410483 | 22368992 | rs9872825 | C | T | 0.9383 | 0.0222 | 1.0224 | 1.0426 |
| | T_C | | | | | | | | | |
| 3 | 3_23684104_ | 23684104 | 23642613 | rs6768220 | A | G | 0.6912 | 0.0000 | 1.0000 | 1.0000 |
| | G_A | | | | | | | | | |
| 3 | 3_24284559_ | 24284559 | 24243068 | 3:24284559_G | G | GAAA | 0.8040 | 0.0037 | 1.0037 | 1.0060 |
| | GAAAAA_G | | | AAAAA_G | | AA | | | | |
| 3 | 3_24308707_ | 24308707 | 24267216 | rs766138533 | T | TAGT | 0.1787 | 0.0056 | 1.0056 | 1.0020 |
| | TAGTC_T | | | | | C | | | | |
| 3 | 3_24958946_ | 24958946 | 24917455 | rs73049478 | G | A | 0.0625 | 0.0138 | 1.0139 | 1.0017 |
| | A_G | | | | | | | | | |
| 3 | 3_25554733_ | 25554733 | 25513242 | rs10446417 | A | G | 0.0714 | −0.0021 | 0.9979 | 0.9997 |
| | G_A | | | | | | | | | |
| 3 | 3_27045315_ | 27045315 | 27003824 | rs11710754 | G | A | 0.3345 | 0.0070 | 1.0070 | 1.0047 |
| | A_G | | | | | | | | | |
| 3 | 3_27116801_ | 27116801 | 27075310 | rs61548819 | T | C | 0.4146 | 0.0000 | 1.0000 | 1.0000 |
| | C_T | | | | | | | | | |
| 3 | 3_27142557_ | 27142557 | 27101066 | | GA | G | 0.4960 | 0.0000 | 1.0000 | 1.0000 |
| | G_GA | | | | | | | | | |
| 3 | 3_27187022_ | 27187022 | 27145531 | rs17317121 | A | G | 0.0583 | 0.0010 | 1.0010 | 1.0001 |
| | G_A | | | | | | | | | |
| 3 | 3_27246771_ | 27246771 | 27205280 | rs34629416 | T | G | 0.2283 | 0.0000 | 1.0000 | 1.0000 |
| | G_T | | | | | | | | | |
| 3 | 3_27256049_ | 27256049 | 27214558 | rs6805966 | T | G | 0.4801 | 0.0044 | 1.0044 | 1.0042 |
| | G_T | | | | | | | | | |
| 3 | 3_27307298_ | 27307298 | 27265807 | 3:27307298_C | C | CT | 0.5032 | 0.0000 | 1.0000 | 1.0000 |
| | CT_C | | | T_C | | | | | | |
| 3 | 3_27347117_ | 27347117 | 27305626 | rs63334461 | AT | A | 0.4680 | 0.0000 | 1.0000 | 1.0000 |
| | A_AT | | | | | | | | | |
| 3 | 3_27353716_ | 27353716 | 27312225 | rs552647 | A | C | 0.5259 | 0.0363 | 1.0370 | 1.0393 |
| | C_A | | | | | | | | | |
| 3 | 3_27368425_ | 27368425 | 27326934 | rs1352941 | T | C | 0.5000 | 0.0000 | 1.0000 | 1.0000 |
| | C_T | | | | | | | | | |
| 3 | 3_27388664_ | 27388664 | 27347173 | rs62255657 | G | C | 0.2735 | 0.0273 | 1.0277 | 1.0152 |
| | C_G | | | | | | | | | |
| 3 | 3_27392625_ | 27392625 | 27351134 | rs73055736 | A | T | 0.4212 | −0.0122 | 0.9879 | 0.9898 |
| | T_A | | | | | | | | | |
| 3 | 3_27413566_ | 27413566 | 27372075 | rs4973766 | T | C | 0.4568 | 0.0000 | 1.0000 | 1.0000 |
| | C_T | | | | | | | | | |
| 3 | 3_27432206_ | 27432206 | 27390715 | rs6551196 | C | T | 0.5611 | 0.0092 | 1.0092 | 1.0104 |
| | T_C | | | | | | | | | |
| 3 | 3_27520500_ | 27520500 | 27479009 | rs12054243 | G | A | 0.3612 | −0.0015 | 0.9985 | 0.9989 |
| | A_G | | | | | | | | | |
| 3 | 3_27785427_ | 27785427 | 27743936 | rs74614983 | C | T | 0.0217 | −0.0028 | 0.9972 | 0.9999 |
| | T_C | | | | | | | | | |
| 3 | 3_27817597_ | 27817597 | 27776106 | rs141629957 | A | G | 0.0170 | −0.0054 | 0.9946 | 0.9998 |
| | G_A | | | | | | | | | |
| 3 | 3_27830509_ | 27830509 | 27789018 | rs12491073 | A | G | 0.1494 | −0.0001 | 0.9999 | 1.0000 |
| | G_A | | | | | | | | | |
| 3 | 3_29229732_ | 29229732 | 29188241 | rs73058397 | G | A | 0.0276 | 0.0000 | 1.0000 | 1.0000 |
| | A_G | | | | | | | | | |
| 3 | 3_29254155_ | 29254155 | 29212664 | rs111292208 | C | T | 0.0259 | 0.0000 | 1.0000 | 1.0000 |
| | T_C | | | | | | | | | |
| 3 | 3_29294845_ | 29294845 | 29253354 | rs112476261 | T | C | 0.0163 | −0.0497 | 0.9515 | 0.9984 |
| | C_T | | | | | | | | | |
| 3 | 3_29359952_ | 29359952 | 29318461 | rs57440162 | C | T | 0.3673 | 0.0044 | 1.0044 | 1.0032 |
| | T_C | | | | | | | | | |
| 3 | 3_30283362_ | 30283362 | 30241871 | rs17564259 | T | C | 0.0727 | −0.0017 | 0.9983 | 0.9998 |
| | C_T | | | | | | | | | |
| 3 | 3_30307462_ | 30307462 | 30265971 | rs112847401 | A | G | 0.0603 | −0.0083 | 0.9917 | 0.9990 |
| | G_A | | | | | | | | | |
| 3 | 3_30549108_ | 30549108 | 30507616 | rs73050822 | A | G | 0.1014 | −0.0014 | 0.9986 | 0.9997 |
| | G_A | | | | | | | | | |
| 3 | 3_30582035_ | 30582035 | 30540543 | rs150163739 | T | C | 0.0236 | 0.0090 | 1.0090 | 1.0004 |
| | C_T | | | | | | | | | |
| 3 | 3_30664321_ | 30664321 | 30622829 | rs7644802 | A | G | 0.4627 | −0.0027 | 0.9973 | 0.9975 |
| | G_A | | | | | | | | | |
| 3 | 3_30670425_ | 30670425 | 30628933 | rs5020833 | C | G | 0.3217 | 0.0000 | 1.0000 | 1.0000 |
| | G_C | | | | | | | | | |
| 3 | 3_30675880_ | 30675880 | 30634388 | rs1431131 | T | A | 0.3596 | 0.0025 | 1.0025 | 1.0018 |
| | A_T | | | | | | | | | |
| 3 | 3_30677116_ | 30677116 | 30635624 | rs952411 | A | G | 0.6135 | 0.0032 | 1.0032 | 1.0039 |
| | G_A | | | | | | | | | |
| 3 | 3_30684097_ | 30684097 | 30642605 | rs35263707 | A | G | 0.3253 | 0.0108 | 1.0109 | 1.0071 |
| | G_A | | | | | | | | | |
| 3 | 3_30684907_ | 30684907 | 30643415 | rs17838698 | T | C | 0.2975 | 0.0108 | 1.0109 | 1.0065 |
| | C_T | | | | | | | | | |
| 3 | 3_30689303_ | 30689303 | 30647811 | rs7613040 | G | T | 0.6414 | 0.0031 | 1.0031 | 1.0040 |
| | T_G | | | | | | | | | |
| 3 | 3_35009348_ | 35009348 | 34967856 | rs75736174 | A | G | 0.0939 | 0.0032 | 1.0032 | 1.0006 |
| | G_A | | | | | | | | | |
| 3 | 3_35113193_ | 35113193 | 35071701 | rs11129635 | T | C | 0.0944 | 0.0022 | 1.0022 | 1.0004 |
| | C_T | | | | | | | | | |
| 3 | 3_37516184_ | 37516184 | 37474693 | rs2162683 | T | C | 0.3410 | 0.0000 | 1.0000 | 1.0000 |
| | C_T | | | | | | | | | |
| 3 | 3_46532543_ | 46532543 | 46491053 | rs79600837 | T | C | 0.1402 | 0.0043 | 1.0043 | 1.0012 |
| | C_T | | | | | | | | | |
| 3 | 3_46687519_ | 46687519 | 46646029 | rs13319345 | T | C | 0.3520 | −0.0042 | 0.9958 | 0.9971 |
| | C_T | | | | | | | | | |
| 3 | 3_46866866_ | 46866866 | 46825376 | rs6796502 | A | G | 0.1008 | −0.0278 | 0.9726 | 0.9945 |
| | G_A | | | | | | | | | |
| 3 | 3_46891310_ | 46891310 | 46849820 | rs57459864 | C | G | 0.1576 | −0.0113 | 0.9888 | 0.9965 |
| | G_C | | | | | | | | | |
| 3 | 3_47210979_ | 47210979 | 47169489 | rs184445588 | T | G | 0.0139 | −0.0062 | 0.9938 | 0.9998 |
| | G_T | | | | | | | | | |
| 3 | 3_47695680_ | 47695680 | 47654190 | rs186117953 | G | T | 0.0104 | −0.0136 | 0.9865 | 0.9997 |
| | T_G | | | | | | | | | |
| 3 | 3_48007463_ | 48007463 | 47965973 | rs75420707 | T | C | 0.0271 | 0.0000 | 1.0000 | 1.0000 |
| | C_T | | | | | | | | | |
| 3 | 3_48261969_ | 48261969 | 48220479 | rs36019664 | T | C | 0.0277 | −0.0098 | 0.9902 | 0.9995 |
| | C_T | | | | | | | | | |
| 3 | 3_48294816_ | 48294816 | 48253326 | rs35010013 | G | C | 0.0399 | −0.0131 | 0.9870 | 0.9990 |
| | C_G | | | | | | | | | |
| 3 | 3_48839507_ | 48839507 | 48802074 | rs145663361 | A | T | 0.0148 | −0.0263 | 0.9740 | 0.9992 |
| | T_A | | | | | | | | | |
| 3 | 3_49166460_ | 49166460 | 49129027 | rs61729152 | T | C | 0.0214 | −0.0138 | 0.9863 | 0.9994 |
| | C_T | | | | | | | | | |
| 3 | 3_49518299_ | 49518299 | 49480866 | rs71615484 | G | A | 0.3599 | −0.0084 | 0.9916 | 0.9940 |
| | A_G | | | | | | | | | |
| 3 | 3_49710514_ | 49710514 | 49673081 | 3:49710514_C | C | CCGG | 0.2870 | −0.0093 | 0.9907 | 0.9947 |
| | CCGGG_C | | | CGGG_C | | G | | | | |
| 3 | 3_49789921_ | 49789921 | 49752488 | rs547649546 | CA | C | 0.5004 | −0.0046 | 0.9954 | 0.9954 |
| | C_CA | | | | | | | | | |
| 3 | 3_49843723_ | 49843723 | 49806290 | rs3819325 | C | T | 0.4751 | 0.0000 | 1.0000 | 1.0000 |
| | T_C | | | | | | | | | |
| 3 | 3_52529317_ | 52529317 | 52495301 | rs79914724 | T | C | 0.0121 | 0.0000 | 1.0000 | 1.0000 |
| | C_T | | | | | | | | | |
| 3 | 3_53217413_ | 53217413 | 53183397 | rs2306572 | G | A | 0.2391 | −0.0059 | 0.9941 | 0.9972 |
| | A_G | | | | | | | | | |
| 3 | 3_53846588_ | 53846588 | 53812561 | 3:53846588_T | T | TTTT | 0.0192 | −0.0197 | 0.9805 | 0.9993 |
| | TTTTG_T | | | TTTG_T | | G | | | | |
| 3 | 3_54648537_ | 54648537 | 54614510 | rs34592432 | C | A | 0.1662 | 0.0021 | 1.0021 | 1.0007 |
| | A_C | | | | | | | | | |
| 3 | 3_54695858_ | 54695858 | 54661831 | rs75606245 | G | C | 0.0994 | 0.0067 | 1.0067 | 1.0013 |
| | C_G | | | | | | | | | |
| 3 | 3_55072187_ | 55072187 | 55038160 | rs358493 | T | C | 0.3416 | 0.0061 | 1.0061 | 1.0042 |
| | C_T | | | | | | | | | |
| 3 | 3_55553097_ | 55553097 | 55519069 | rs62251462 | T | C | 0.0465 | −0.0100 | 0.9900 | 0.9991 |
| | C_T | | | | | | | | | |
| 3 | 3_55561657_ | 55561657 | 55527629 | rs62251466 | G | A | 0.0361 | −0.0067 | 0.9933 | 0.9995 |
| | A_G | | | | | | | | | |
| 3 | 3_55970777_ | 55970777 | 55936749 | rs138866686 | AT | A | 0.0305 | −0.0327 | 0.9678 | 0.9980 |
| | A_AT | | | | | | | | | |
| 3 | 3_59361172_ | 59361172 | 59375446 | rs11429792 | CT | C | 0.6917 | 0.0077 | 1.0077 | 1.0107 |
| | C_CT | | | | | | | | | |
| 3 | 3_59373745_ | 59373745 | 59388019 | rs2886671 | T | C | 0.4294 | −0.0127 | 0.9874 | 0.9892 |
| | C_T | | | | | | | | | |
| 3 | 3_59693450_ | 59693450 | 59707724 | rs56915453 | A | C | 0.1236 | 0.0043 | 1.0043 | 1.0011 |
| | C_A | | | | | | | | | |
| 3 | 3_59771038_ | 59771038 | 59785312 | 3:59771038_C | C | CT | 0.7385 | 0.0020 | 1.0020 | 1.0030 |
| | CT_C | | | TC | | | | | | |
| 3 | 3_59832595_ | 59832595 | 59846869 | rs633189 | G | C | 0.6362 | 0.0117 | 1.0118 | 1.0150 |
| | C_G | | | | | | | | | |
| 3 | 3_59856766_ | 59856766 | 59871040 | rs57341675 | C | T | 0.1136 | −0.0018 | 0.9982 | 0.9996 |
| | T_C | | | | | | | | | |
| 3 | 3_60553783_ | 60553783 | 60568050 | rs4679551 | G | A | 0.1666 | −0.0059 | 0.9941 | 0.9980 |
| | A_G | | | | | | | | | |
| 3 | 3_61466213_ | 61466213 | 61480539 | rs1975730 | G | C | 0.8646 | −0.0044 | 0.9956 | 0.9924 |
| | C_G | | | | | | | | | |
| 3 | 3_61486355_ | 61486355 | 61500681 | 3:61486355_A | A | AAG | 0.1962 | 0.0089 | 1.0089 | 1.0035 |
| | AAG_A | | | AG_A | | | | | | |
| 3 | 3_62650762_ | 62650762 | 62665087 | rs62242517 | T | C | 0.1347 | 0.0000 | 1.0000 | 1.0000 |
| | C_T | | | | | | | | | |
| 3 | 3_63491051_ | 63491051 | 63505375 | rs17068857 | G | A | 0.1693 | −0.0075 | 0.9925 | 0.9975 |
| | A_G | | | | | | | | | |
| 3 | 3_63493054_ | 63493054 | 63507378 | rs71743220 | GA | G | 0.2023 | −0.0067 | 0.9933 | 0.9973 |
| | G_GA | | | | | | | | | |
| 3 | 3_63743823_ | 63743823 | 63758147 | rs74886078 | C | T | 0.0215 | −0.0065 | 0.9935 | 0.9997 |
| | T_C | | | | | | | | | |
| 3 | 3_63887449_ | 63887449 | 63901773 | | TTG | T | 0.1297 | 0.0166 | 1.0167 | 1.0043 |
| | T_TTG | | | | | | | | | |
| 3 | 3_63986046_ | 63986046 | 64000370 | 3:63986046_C | C | CT | 0.1669 | 0.0132 | 1.0133 | 1.0044 |
| | CT_C | | | TC | | | | | | |
| 3 | 3_64019206_ | 64019206 | 64033530 | rs6785145 | G | A | 0.4264 | 0.0040 | 1.0040 | 1.0034 |
| | A_G | | | | | | | | | |
| 3 | 3_64029383_ | 64029383 | 64043707 | rs831574 | G | A | 0.3435 | 0.0000 | 1.0000 | 1.0000 |
| | A_G | | | | | | | | | |
| 3 | 3_64116494_ | 64116494 | 64130818 | rs55796057 | G | T | 0.1891 | 0.0009 | 1.0009 | 1.0003 |
| | T_G | | | | | | | | | |
| 3 | 3_64182827_ | 64182827 | 64197151 | rs57476258 | A | G | 0.1613 | 0.0041 | 1.0041 | 1.0013 |
| | G_A | | | | | | | | | |
| 3 | 3_64370063_ | 64370063 | 64384387 | rs7653442 | G | T | 0.2405 | 0.0069 | 1.0069 | 1.0033 |
| | T_G | | | | | | | | | |
| 3 | 3_64371596_ | 64371596 | 64385920 | rs144435498 | CGGTT | C | 0.1179 | 0.0056 | 1.0056 | 1.0013 |
| | C_CGGTT | | | | | | | | | |
| 3 | 3_65461535_ | 65461535 | 65475860 | rs7614228 | T | C | 0.2396 | −0.0076 | 0.9924 | 0.9964 |
| | C_T | | | | | | | | | |
| 3 | 3_65641268_ | 65641268 | 65655593 | rs1156024 | C | G | 0.3312 | 0.0067 | 1.0067 | 1.0045 |
| | G_C | | | | | | | | | |
| 3 | 3_65650331_ | 65650331 | 65664656 | rs36091678 | G | T | 0.3453 | 0.0006 | 1.0006 | 1.0004 |
| | T_G | | | | | | | | | |
| 3 | 3_66458047_ | 66458047 | 66407623 | rs796133473 | GCA | G | 0.5826 | 0.0086 | 1.0086 | 1.0101 |
| | G_GCA | | | | | | | | | |
| 3 | 3_67454299_ | 67454299 | 67403875 | rs55672035 | C | T | 0.4504 | −0.0066 | 0.9934 | 0.9941 |
| | T_C | | | | | | | | | |
| 3 | 3_71476825_ | 71476825 | 71427674 | rs6549389 | C | G | 0.7313 | −0.0046 | 0.9954 | 0.9933 |
| | G_C | | | | | | | | | |
| 3 | 3_71531467_ | 71531467 | 71482316 | rs35483362 | C | T | 0.4758 | −0.0123 | 0.9878 | 0.9884 |
| | T_C | | | | | | | | | |
| 3 | 3_72334204_ | 72334204 | 72285053 | 3:72334204_C | C | CCTC | 0.4389 | 0.0037 | 1.0037 | 1.0033 |
| | CCTCCCAA_C | | | CTCCCAA_C | | CCAA | | | | |
| 3 | 3_72352464_ | 72352464 | 72303313 | | TACACAT | T | 0.4850 | 0.0000 | 1.0000 | 1.0000 |
| | T_TACACATGG | | | | GGCCACC | | | | | |
| | CCACCGA | | | | GA | | | | | |
| 3 | 3_72457221_ | 72457221 | 72408070 | rs6549455 | G | A | 0.6395 | 0.0087 | 1.0087 | 1.0112 |
| | A_G | | | | | | | | | |
| 3 | 3_73330742_ | 73330742 | 73281591 | rs112563751 | A | G | 0.0362 | 0.0000 | 1.0000 | 1.0000 |
| | G_A | | | | | | | | | |
| 3 | 3_73343559_ | 73343559 | 73294408 | rs7647925 | C | A | 0.5262 | −0.0136 | 0.9865 | 0.9858 |
| | A_C | | | | | | | | | |
| 3 | 3_80126605_ | 80126605 | 80077455 | rs73128241 | C | T | 0.0264 | 0.0038 | 1.0038 | 1.0002 |
| | T_C | | | | | | | | | |
| 3 | 3_81234859_ | 81234859 | 81185708 | rs1477676 | T | G | 0.7525 | −0.0102 | 0.9899 | 0.9848 |
| | G_T | | | | | | | | | |
| 3 | 3_85724423_ | 85724423 | 85675273 | rs147473479 | G | A | 0.0115 | 0.0423 | 1.0432 | 1.0010 |
| | A_G | | | | | | | | | |
| 3 | 3_85768032_ | 85768032 | 85718882 | 3:85768032_T | T | TTTA | 0.3170 | 0.0004 | 1.0004 | 1.0003 |
| | TTTATTATTAT | | | TTATTATTATT | | TTAT | | | | |
| | TATTA_T | | | ATTA_T | | TATT | | | | |
| | | | | | | ATTA | | | | |
| 3 | 3_86195124_ | 86195124 | 86145974 | rs6549075 | G | A | 0.6825 | 0.0040 | 1.0040 | 1.0055 |
| | A_G | | | | | | | | | |
| 3 | 3_86203809_ | 86203809 | 86154659 | rs9833726 | T | G | 0.1383 | −0.0112 | 0.9889 | 0.9969 |
| | G_T | | | | | | | | | |
| 3 | 3_86499424_ | 86499424 | 86450274 | rs148727437 | A | G | 0.0148 | 0.0000 | 1.0000 | 1.0000 |
| | G_A | | | | | | | | | |
| 3 | 3_87037543_ | 87037543 | 86988393 | rs13066793 | G | A | 0.0921 | −0.0277 | 0.9727 | 0.9950 |
| | A_G | | | | | | | | | |
| 3 | 3_87842066_ | 87842066 | 87792916 | rs1873209 | C | T | 0.3376 | 0.0075 | 1.0075 | 1.0051 |
| | T_C | | | | | | | | | |
| 3 | 3_87995951_ | 87995951 | 87946801 | rs2915293 | A | G | 0.6973 | −0.0014 | 0.9986 | 0.9980 |
| | G_A | | | | | | | | | |
| 3 | 3_88196211_ | 88196211 | 88147061 | rs9861443 | C | A | 0.7295 | −0.0045 | 0.9955 | 0.9935 |
| | A_C | | | | | | | | | |
| 3 | 3_98830472_ | 98830472 | 99111628 | rs34972210 | G | A | 0.1172 | 0.0076 | 1.0076 | 1.0018 |
| | A_G | | | | | | | | | |
| 3 | 3_99184544_ | 99184544 | 99465700 | rs2700628 | T | C | 0.0955 | 0.0012 | 1.0012 | 1.0002 |
| | C_T | | | | | | | | | |
| 3 | 3_99212523_ | 99212523 | 99493679 | rs35583542 | T | G | 0.1039 | 0.0036 | 1.0036 | 1.0007 |
| | G_T | | | | | | | | | |
| 3 | 3_99227951_ | 99227951 | 99509107 | rs115575016 | T | A | 0.2760 | 0.0011 | 1.0011 | 1.0006 |
| | A_T | | | | | | | | | |
| 3 | 3_99269750_ | 99269750 | 99550906 | rs1513304 | C | T | 0.2829 | 0.0014 | 1.0014 | 1.0008 |
| | T_C | | | | | | | | | |
| 3 | 3_99275637_ | 99275637 | 99556793 | rs71313557 | A | G | 0.0373 | 0.0032 | 1.0032 | 1.0002 |
| | G_A | | | | | | | | | |
| 3 | 3_99303827_ | 99303827 | 99584983 | rs13093075 | C | A | 0.7131 | −0.0078 | 0.9922 | 0.9889 |
| | A_C | | | | | | | | | |
| 3 | 3_99403877_ | 99403877 | 99685033 | rs639355 | A | G | 0.4852 | −0.0094 | 0.9906 | 0.9909 |
| | G_A | | | | | | | | | |
| 3 | 3_99425767_ | 99425767 | 99706923 | rs629120 | T | A | 0.7823 | 0.0000 | 1.0000 | 1.0000 |
| | A_T | | | | | | | | | |
| 3 | 3_99687273_ | 99687273 | 99968429 | rs4361315 | A | G | 0.7359 | −0.0063 | 0.9937 | 0.9908 |
| | G_A | | | | | | | | | |
| 3 | 3_103062567_ | 103062567 | 103343723 | rs73153693 | T | A | 0.0706 | −0.0178 | 0.9824 | 0.9975 |
| | A_T | | | | | | | | | |
| 3 | 3_107112555_ | 107112555 | 107393708 | rs10511262 | G | C | 0.1123 | −0.0054 | 0.9946 | 0.9988 |
| | C_G | | | | | | | | | |
| 3 | 3_110267702_ | 110267702 | 110548855 | rs80282904 | G | T | 0.1047 | −0.0129 | 0.9872 | 0.9973 |
| | T_G | | | | | | | | | |
| 3 | 3_111387513_ | 111387513 | 111668666 | rs146576688 | G | C | 0.0201 | −0.0253 | 0.9750 | 0.9990 |
| | C_G | | | | | | | | | |
| 3 | 3_114117017_ | 114117017 | 114398170 | rs16822588 | G | T | 0.1095 | 0.0059 | 1.0059 | 1.0013 |
| | T_G | | | | | | | | | |
| 3 | 3_115528511_ | 115528511 | 115809664 | rs28636502 | G | A | 0.1254 | −0.0041 | 0.9959 | 0.9990 |
| | A_G | | | | | | | | | |
| 3 | 3_116345449_ | 116345449 | 116626602 | rs6438324 | A | C | 0.4997 | 0.0037 | 1.0037 | 1.0037 |
| | C_A | | | | | | | | | |
| 3 | 3_118666410_ | 118666410 | 118947563 | rs73185814 | T | C | 0.0376 | 0.0000 | 1.0000 | 1.0000 |
| | C_T | | | | | | | | | |
| 3 | 3_118984456_ | 118984456 | 119265609 | rs77424405 | T | A | 0.0188 | 0.0587 | 1.0605 | 1.0023 |
| | A_T | | | | | | | | | |
| 3 | 3_125060843_ | 125060843 | 125341999 | rs6804933 | A | C | 0.7447 | −0.0045 | 0.9955 | 0.9933 |
| | C_A | | | | | | | | | |
| 3 | 3_125074570_ | 125074570 | 125355726 | 3:125074570 | T | TA | 0.6995 | −0.0044 | 0.9956 | 0.9939 |
| | TA_T | | | TA_T | | | | | | |
| 3 | 3_125132642_ | 125132642 | 125413798 | rs62273093 | A | G | 0.7271 | −0.0054 | 0.9946 | 0.9922 |
| | G_A | | | | | | | | | |
| 3 | 3_125833260_ | 125833260 | 126114417 | rs2290053 | T | C | 0.4932 | 0.0031 | 1.0031 | 1.0031 |
| | C_T | | | | | | | | | |
| 3 | 3_125847647_ | 125847647 | 126128804 | rs6775421 | C | T | 0.6555 | 0.0015 | 1.0015 | 1.0020 |
| | T_C | | | | | | | | | |
| 3 | 3_126056772_ | 126056772 | 126337929 | rs7619823 | T | C | 0.4757 | 0.0110 | 1.0111 | 1.0106 |
| | C_T | | | | | | | | | |
| 3 | 3_126174838_ | 126174838 | 126455995 | rs777504 | G | A | 0.9633 | 0.0118 | 1.0119 | 1.0230 |
| | A_G | | | | | | | | | |
| 3 | 3_129060011_ | 129060011 | 129341168 | rs7374197 | G | A | 0.5471 | 0.0082 | 1.0082 | 1.0090 |
| | A_G | | | | | | | | | |
| 3 | 3_129647253_ | 129647253 | 129928410 | rs34941175 | A | G | 0.0624 | −0.0186 | 0.9816 | 0.9977 |
| | G_A | | | | | | | | | |
| 3 | 3_137325391_ | 137325391 | 137606549 | rs9879724 | C | T | 0.1892 | 0.0040 | 1.0040 | 1.0015 |
| | T_C | | | | | | | | | |
| 3 | 3_138723358_ | 138723358 | 139004516 | rs138346232 | A | G | 0.0193 | −0.0431 | 0.9578 | 0.9984 |
| | G_A | | | | | | | | | |
| 3 | 3_139697874_ | 139697874 | 139979032 | rs73224969 | G | A | 0.0375 | 0.0000 | 1.0000 | 1.0000 |
| | A_G | | | | | | | | | |
| 3 | 3_141063953_ | 141063953 | 141345111 | rs9838625 | T | C | 0.4154 | 0.0000 | 1.0000 | 1.0000 |
| | C_T | | | | | | | | | |
| 3 | 3_141112859_ | 141112859 | 141394017 | 3:141112859 | C | CTT | 0.4149 | 0.0258 | 1.0261 | 1.0218 |
| | CTT_C | | | CTT_C | | | | | | |
| 3 | 3_141142691_ | 141142691 | 141423849 | rs6440006 | A | G | 0.4347 | 0.0043 | 1.0043 | 1.0037 |
| | G_A | | | | | | | | | |
| 3 | 3_141344368_ | 141344368 | 141625526 | rs9821686 | T | A | 0.1541 | −0.0093 | 0.9907 | 0.9971 |
| | A_T | | | | | | | | | |
| 3 | 3_141510052_ | 141510052 | 141791210 | rs2681693 | T | G | 0.1509 | −0.0048 | 0.9952 | 0.9986 |
| | G_T | | | | | | | | | |
| 3 | 3_141952067_ | 141952067 | 142233225 | rs10662123 | CAAA | C | 0.4841 | −0.0038 | 0.9962 | 0.9963 |
| | C_CAAA | | | | | | | | | |
| 3 | 3_142863119_ | 142863119 | 143144277 | rs4683748 | A | G | 0.3797 | −0.0061 | 0.9939 | 0.9954 |
| | G_A | | | | | | | | | |
| 3 | 3_142982700_ | 142982700 | 143263858 | rs6440156 | A | G | 0.7758 | 0.0098 | 1.0098 | 1.0153 |
| | G_A | | | | | | | | | |
| 3 | 3_145444094_ | 145444094 | 145726307 | rs62269362 | T | C | 0.3689 | 0.0011 | 1.0011 | 1.0008 |
| | C_T | | | | | | | | | |
| 3 | 3_145884210_ | 145884210 | 146166423 | rs7623263 | C | T | 0.4239 | −0.0028 | 0.9972 | 0.9976 |
| | T_C | | | | | | | | | |
| 3 | 3_148576368_ | 148576368 | 148858581 | rs1845415 | C | A | 0.0183 | −0.0341 | 0.9665 | 0.9988 |
| | A_C | | | | | | | | | |
| 3 | 3_148616863_ | 148616863 | 148899076 | | AAT | A | 0.0368 | −0.0096 | 0.9904 | 0.9993 |
| | A_AAT | | | | | | | | | |
| 3 | 3_150455755_ | 150455755 | 150737968 | rs502941 | A | G | 0.8847 | 0.0089 | 1.0089 | 1.0159 |
| | G_A | | | | | | | | | |
| 3 | 3_151062093_ | 151062093 | 151344305 | | TAA | T | 0.2725 | −0.0081 | 0.9919 | 0.9956 |
| | T_TAA | | | | | | | | | |
| 3 | 3_151079960_ | 151079960 | 151362172 | rs6793061 | G | T | 0.2412 | −0.0044 | 0.9956 | 0.9979 |
| | T_G | | | | | | | | | |
| 3 | 3_151523713_ | 151523713 | 151805925 | rs67337703 | A | G | 0.4022 | 0.0012 | 1.0012 | 1.0010 |
| | G_A | | | | | | | | | |
| 3 | 3_151547130_ | 151547130 | 151829342 | rs9841094 | T | C | 0.7748 | 0.0083 | 1.0083 | 1.0130 |
| | C_T | | | | | | | | | |
| 3 | 3_156357618_ | 156357618 | 156639829 | 3:156357618 | C | CTCT | 0.2518 | −0.0019 | 0.9981 | 0.9990 |
| | CTCTT_C | | | CTCTT_C | | T | | | | |
| 3 | 3_156399180_ | 156399180 | 156681391 | rs3851992 | A | G | 0.4066 | 0.0071 | 1.0071 | 1.0058 |
| | G_A | | | | | | | | | |
| 3 | 3_156480941_ | 156480941 | 156763152 | rs2043108 | G | A | 0.5862 | 0.0007 | 1.0007 | 1.0008 |
| | A_G | | | | | | | | | |
| 3 | 3_156525396_ | 156525396 | 156807607 | rs1430411 | A | T | 0.6149 | 0.0041 | 1.0041 | 1.0051 |
| | T_A | | | | | | | | | |
| 3 | 3_158019780_ | 158019780 | 158301991 | rs759541351 | G | GT | 0.5195 | 0.0068 | 1.0068 | 1.0071 |
| | GT_G | | | | | | | | | |
| 3 | 3_158168120_ | 158168120 | 158450331 | rs144232662 | C | CT | 0.2501 | 0.0000 | 1.0000 | 1.0000 |
| | CT_C | | | | | | | | | |
| 3 | 3_162135821_ | 162135821 | 162418033 | rs2405502 | G | A | 0.6713 | 0.0021 | 1.0021 | 1.0028 |
| | A_G | | | | | | | | | |
| 3 | 3_163050198_ | 163050198 | 163332410 | rs77632248 | G | A | 0.0231 | −0.0366 | 0.9641 | 0.9983 |
| | A_G | | | | | | | | | |
| 3 | 3_166739585_ | 166739585 | 167021797 | rs2203919 | A | G | 0.3994 | 0.0005 | 1.0005 | 1.0004 |
| | G_A | | | | | | | | | |
| 3 | 3_168674494_ | 168674494 | 168956706 | rs6781201 | G | A | 0.1388 | 0.0050 | 1.0050 | 1.0014 |
| | A_G | | | | | | | | | |
| 3 | 3_170537091_ | 170537091 | 170819302 | rs12054015 | T | A | 0.3180 | 0.0000 | 1.0000 | 1.0000 |
| | A_T | | | | | | | | | |
| 3 | 3_170625806_ | 170625806 | 170908017 | rs2271998 | T | C | 0.4027 | 0.0000 | 1.0000 | 1.0000 |
| | C_T | | | | | | | | | |
| 3 | 3_172081359_ | 172081359 | 172363569 | rs12152414 | A | G | 0.0329 | 0.0025 | 1.0025 | 1.0002 |
| | G_A | | | | | | | | | |
| 3 | 3_172160206_ | 172160206 | 172442416 | rs114441197 | C | T | 0.0117 | 0.0108 | 1.0109 | 1.0003 |
| | T_C | | | | | | | | | |
| 3 | 3_172204644_ | 172204644 | 172486854 | rs1597088 | G | A | 0.4829 | −0.0045 | 0.9955 | 0.9957 |
| | A_G | | | | | | | | | |
| 3 | 3_172222756_ | 172222756 | 172504966 | rs7631904 | T | C | 0.2109 | 0.0000 | 1.0000 | 1.0000 |
| | C_T | | | | | | | | | |
| 3 | 3_172227766_ | 172227766 | 172509976 | 3:172227766_ | C | CA | 0.3763 | −0.0054 | 0.9946 | 0.9960 |
| | CA_C | | | CA_C | | | | | | |
| 3 | 3_172237859_ | 172237859 | 172520069 | rs3136588 | T | G | 0.0411 | 0.0000 | 1.0000 | 1.0000 |
| | G_T | | | | | | | | | |
| 3 | 3_172285237_ | 172285237 | 172567447 | rs58058861 | A | G | 0.2131 | 0.0105 | 1.0106 | 1.0045 |
| | G_A | | | | | | | | | |
| 3 | 3_172598961_ | 172598961 | 172881171 | rs2088658 | T | C | 0.7522 | −0.0048 | 0.9952 | 0.9928 |
| | C_T | | | | | | | | | |
| 3 | 3_172611899_ | 172611899 | 172894109 | rs10451925 | C | T | 0.6812 | −0.0015 | 0.9985 | 0.9980 |
| | T_C | | | | | | | | | |
| 3 | 3_172624366_ | 172624366 | 172906576 | rs34346181 | CTA | C | 0.7093 | −0.0046 | 0.9954 | 0.9935 |
| | C_CTA | | | | | | | | | |
| 3 | 3_173220074_ | 173220074 | 173502284 | rs550880 | T | G | 0.1100 | 0.0122 | 1.0123 | 1.0027 |
| | G_T | | | | | | | | | |
| 3 | 3_175862678_ | 175862678 | 176144890 | rs2902242 | C | A | 0.1736 | 0.0047 | 1.0047 | 1.0016 |
| | A_C | | | | | | | | | |
| 3 | 3_175914226_ | 175914226 | 176196438 | rs62285838 | T | C | 0.1394 | 0.0164 | 1.0165 | 1.0046 |
| | C_T | | | | | | | | | |
| 3 | 3_176852038_ | 176852038 | 177134250 | rs7626218 | T | A | 0.3982 | 0.0091 | 1.0091 | 1.0073 |
| | A_T | | | | | | | | | |
| 3 | 3_177427650_ | 177427650 | 177709862 | rs2133590 | G | A | 0.3602 | −0.0038 | 0.9962 | 0.9973 |
| | A_G | | | | | | | | | |
| 3 | 3_177522037_ | 177522037 | 177804249 | rs35370551 | C | T | 0.1284 | 0.0009 | 1.0009 | 1.0002 |
| | T_C | | | | | | | | | |
| 3 | 3_177753655_ | 177753655 | 178035867 | rs115452163 | T | G | 0.0288 | 0.0221 | 1.0223 | 1.0013 |
| | G_T | | | | | | | | | |
| 3 | 3_179010099_ | 179010099 | 179292311 | 3:179010099_ | T | TA | 0.6072 | −0.0046 | 0.9954 | 0.9944 |
| | TA_T | | | TA_T | | | | | | |
| 3 | 3_179020085_ | 179020085 | 179302297 | rs12634043 | C | T | 0.6502 | −0.0050 | 0.9950 | 0.9935 |
| | T_C | | | | | | | | | |
| 3 | 3_179673801_ | 179673801 | 179956013 | rs13097429 | G | A | 0.1355 | 0.0000 | 1.0000 | 1.0000 |
| | A_G | | | | | | | | | |
| 3 | 3_182428424_ | 182428424 | 182710636 | rs77442660 | A | G | 0.0872 | −0.0132 | 0.9869 | 0.9977 |
| | G_A | | | | | | | | | |
| 3 | 3_183178910_ | 183178910 | 183461122 | rs11707996 | A | G | 0.2023 | −0.0097 | 0.9903 | 0.9961 |
| | G_A | | | | | | | | | |
| 3 | 3_183184794_ | 183184794 | 183467006 | rs3074830 | C | CTTT | 0.2300 | −0.0015 | 0.9985 | 0.9993 |
| | CTTTTTTT_C | | | | | TTTT | | | | |
| 3 | 3_183888015_ | 183888015 | 184170227 | rs147393618 | A | G | 0.0265 | 0.0145 | 1.0146 | 1.0008 |
| | G_A | | | | | | | | | |
| 3 | 3_185741991_ | 185741991 | 186024202 | rs12330780 | A | G | 0.4783 | 0.0036 | 1.0036 | 1.0035 |
| | G_A | | | | | | | | | |
| 3 | 3_185824151_ | 185824151 | 186106362 | rs77195939 | C | G | 0.0487 | −0.0016 | 0.9984 | 0.9998 |
| | G_C | | | | | | | | | |
| 3 | 3_185856621_ | 185856621 | 186138832 | rs57374641 | C | A | 0.0892 | −0.0155 | 0.9846 | 0.9973 |
| | A_C | | | | | | | | | |
| 3 | 3_185964764_ | 185964764 | 186246975 | rs6792471 | T | C | 0.9307 | 0.0110 | 1.0111 | 1.0207 |
| | C_T | | | | | | | | | |
| 3 | 3_185966982_ | 185966982 | 186249193 | rs6799575 | G | C | 0.0378 | −0.0077 | 0.9923 | 0.9994 |
| | C_G | | | | | | | | | |
| 3 | 3_185991240_ | 185991240 | 186273451 | rs9290831 | T | C | 0.0811 | −0.0105 | 0.9896 | 0.9983 |
| | C_T | | | | | | | | | |
| 3 | 3_187310352_ | 187310352 | 187592564 | rs9868558 | A | C | 0.2997 | 0.0073 | 1.0073 | 1.0044 |
| | C_A | | | | | | | | | |
| 3 | 3_187421181_ | 187421181 | 187703393 | rs11708364 | T | C | 0.6379 | 0.0044 | 1.0044 | 1.0056 |
| | C_T | | | | | | | | | |
| 3 | 3_187459779_ | 187459779 | 187741991 | rs3774300 | T | C | 0.7900 | 0.0092 | 1.0092 | 1.0147 |
| | C_T | | | | | | | | | |
| 3 | 3_187756755_ | 187756755 | 188038967 | rs2378359 | A | G | 0.8122 | −0.0001 | 0.9999 | 0.9998 |
| | G_A | | | | | | | | | |
| 3 | 3_188976010_ | 188976010 | 189258221 | rs6801108 | T | C | 0.1820 | 0.0000 | 1.0000 | 1.0000 |
| | C_T | | | | | | | | | |
| 3 | 3_189774456_ | 189774456 | 190056667 | rs9882792 | T | C | 0.2235 | −0.0144 | 0.9857 | 0.9936 |
| | C_T | | | | | | | | | |
| 3 | 3_189816756_ | 189816756 | 190098967 | rs710586 | T | C | 0.1568 | −0.0031 | 0.9969 | 0.9990 |
| | C_T | | | | | | | | | |
| 3 | 3_191507120_ | 191507120 | 191789331 | rs75772781 | C | T | 0.0586 | 0.0001 | 1.0001 | 1.0000 |
| | T_C | | | | | | | | | |
| 3 | 3_192380630_ | 192380630 | 192662841 | rs6784363 | T | G | 0.6051 | 0.0048 | 1.0048 | 1.0058 |
| | G_T | | | | | | | | | |
| 3 | 3_192549113_ | 192549113 | 192831324 | rs7622135 | A | G | 0.4785 | 0.0015 | 1.0015 | 1.0014 |
| | G_A | | | | | | | | | |
| 3 | 3 192569458_ | 192569458 | 192851669 | rs201095372 | A | AT | 0.4894 | 0.0024 | 1.0024 | 1.0024 |
| | AT_A | | | | | | | | | |
| 3 | 3_193701697_ | 193701697 | 193983908 | rs77474546 | G | A | 0.0892 | 0.0065 | 1.0065 | 1.0012 |
| | A_G | | | | | | | | | |
| 3 | 3_195865387_ | 195865387 | 196138516 | rs7634650 | A | G | 0.1730 | 0.0098 | 1.0098 | 1.0034 |
| | G_A | | | | | | | | | |
| 3 | 3_196104863_ | 196104863 | 196377992 | rs62409217 | T | C | 0.1250 | 0.0104 | 1.0105 | 1.0026 |
| | C_T | | | | | | | | | |
| 3 | 3_197256058_ | 197256058 | 197529187 | rs11925979 | T | C | 0.1169 | −0.0030 | 0.9970 | 0.9993 |
| | C_T | | | | | | | | | |
| 3 | 3_197683078_ | 197683078 | 197956207 | 3:197683078_ | C | CCT | 0.1995 | 0.0034 | 1.0034 | 1.0014 |
| | CCT_C | | | CCT_C | | | | | | |
| 4 | 4_560897_ | 560897 | 567108 | rs73074224 | T | A | 0.0434 | 0.0207 | 1.0209 | 1.0018 |
| | A_T | | | | | | | | | |
| 4 | 4_1158991_ | 1158991 | 1165203 | rs17802266 | A | C | 0.2452 | −0.0068 | 0.9932 | 0.9967 |
| | C_A | | | | | | | | | |
| 4 | 4_6922022_ | 6922022 | 6920295 | rs116837201 | G | A | 0.0128 | 0.0051 | 1.0051 | 1.0001 |
| | A_G | | | | | | | | | |
| 4 | 4_7292894_ | 7292894 | 7291167 | rs3846424 | C | T | 0.7343 | 0.0035 | 1.0035 | 1.0052 |
| | T_C | | | | | | | | | |
| 4 | 4_7892228_ | 7892228 | 7890501 | rs55824910 | A | T | 0.1545 | −0.0073 | 0.9927 | 0.9978 |
| | T_A | | | | | | | | | |
| 4 | 4_7932704_ | 7932704 | 7930977 | rs4696808 | T | C | 0.4867 | 0.0063 | 1.0063 | 1.0062 |
| | C_T | | | | | | | | | |
| 4 | 4_7951955_ | 7951955 | 7950228 | 4:7951955_GT | G | GT | 0.0311 | 0.0217 | 1.0219 | 1.0014 |
| | GT_G | | | _G | | | | | | |
| 4 | 4_8321215_ | 8321215 | 8319488 | rs4696811 | A | C | 0.8273 | 0.0050 | 1.0050 | 1.0083 |
| | C_A | | | | | | | | | |
| 4 | 4_9575536_ | 9575536 | 9573912 | rs35535759 | GT | G | 0.2572 | −0.0112 | 0.9889 | 0.9943 |
| | G_GT | | | | | | | | | |
| 4 | 4_10625018_ | 10625018 | 10623394 | rs2720360 | G | C | 0.8126 | 0.0053 | 1.0053 | 1.0087 |
| | C_G | | | | | | | | | |
| 4 | 4_12230032_ | 12230032 | 12228408 | rs62296431 | A | G | 0.0186 | 0.0311 | 1.0316 | 1.0012 |
| | G_A | | | | | | | | | |
| 4 | 4_15011508_ | 15011508 | 15009884 | rs2643087 | T | C | 0.0309 | −0.0134 | 0.9867 | 0.9992 |
| | C_T | | | | | | | | | |
| 4 | 4_17042582_ | 17042582 | 17040959 | rs73245378 | T | C | 0.2395 | 0.0077 | 1.0077 | 1.0037 |
| | C_T | | | | | | | | | |
| 4 | 4_17061476_ | 17061476 | 17059853 | rs828162 | G | C | 0.9141 | −0.0082 | 0.9918 | 0.9851 |
| | C_G | | | | | | | | | |
| 4 | 4_17308014_ | 17308014 | 17306391 | rs73230775 | G | T | 0.1377 | −0.0109 | 0.9892 | 0.9970 |
| | T_G | | | | | | | | | |
| 4 | 4_17315910_ | 17315910 | 17314287 | rs13126451 | C | T | 0.6324 | 0.0035 | 1.0035 | 1.0044 |
| | T_C | | | | | | | | | |
| 4 | 4_17386550_ | 17386550 | 17384927 | rs2031007 | G | A | 0.2353 | 0.0049 | 1.0049 | 1.0023 |
| | A_G | | | | | | | | | |
| 4 | 4_18675840_ | 18675840 | 18674217 | rs4428281 | G | A | 0.9536 | 0.0126 | 1.0127 | 1.0243 |
| | A_G | | | | | | | | | |
| 4 | 4_18753395_ | 18753395 | 18751772 | rs1568226 | T | C | 0.7818 | 0.0095 | 1.0095 | 1.0150 |
| | C_T | | | | | | | | | |
| 4 | 4_20064344_ | 20064344 | 20062721 | rs201232508 | TA | T | 0.7154 | −0.0056 | 0.9944 | 0.9920 |
| | T_TA | | | | | | | | | |
| 4 | 4_20263484_ | 20263484 | 20261861 | rs150694043 | C | G | 0.0105 | −0.0442 | 0.9568 | 0.9991 |
| | G_C | | | | | | | | | |
| 4 | 4_21194184_ | 21194184 | 21192561 | rs17514618 | G | C | 0.0271 | −0.0075 | 0.9925 | 0.9996 |
| | C_G | | | | | | | | | |
| 4 | 4_22660580_ | 22660580 | 22658957 | rs141487006 | T | C | 0.0125 | −0.0295 | 0.9709 | 0.9993 |
| | C_T | | | | | | | | | |
| 4 | 4_23813109_ | 23813109 | 23811486 | rs7667050 | C | T | 0.5279 | 0.0058 | 1.0058 | 1.0062 |
| | T_C | | | | | | | | | |
| 4 | 4_23853011_ | 23853011 | 23851388 | rs7665116 | C | T | 0.1309 | 0.0089 | 1.0089 | 1.0023 |
| | T_C | | | | | | | | | |
| 4 | 4_24209070_ | 24209070 | 24207447 | rs113743277 | A | G | 0.0100 | −0.0034 | 0.9966 | 0.9999 |
| | G_A | | | | | | | | | |
| 4 | 4_27680810_ | 27680810 | 27679188 | rs34258956 | G | A | 0.0166 | 0.0224 | 1.0227 | 1.0008 |
| | A_G | | | | | | | | | |
| 4 | 4_27906865_ | 27906865 | 27905243 | rs1038442 | C | T | 0.4591 | 0.0109 | 1.0110 | 1.0101 |
| | T_C | | | | | | | | | |
| 4 | 4_31213252_ | 31213252 | 31211630 | rs2133514 | A | G | 0.0873 | 0.0051 | 1.0051 | 1.0009 |
| | G_A | | | | | | | | | |
| 4 | 4_31229344_ | 31229344 | 31227722 | | CTTT | CT | 0.0992 | 0.0000 | 1.0000 | 1.0000 |
| | CT_CTTT | | | | | | | | | |
| 4 | 4_31255491_ | 31255491 | 31253869 | rs78953893 | A | C | 0.1294 | 0.0024 | 1.0024 | 1.0006 |
| | C_A | | | | | | | | | |
| 4 | 4_31478536_ | 31478536 | 31476914 | rs410175 | A | C | 0.8191 | 0.0053 | 1.0053 | 1.0087 |
| | C_A | | | | | | | | | |
| 4 | 4_32552249_ | 32552249 | 32550627 | rs17081562 | A | T | 0.0142 | 0.0228 | 1.0231 | 1.0007 |
| | T_A | | | | | | | | | |
| 4 | 4_38571221_ | 38571221 | 38569600 | rs11930931 | G | A | 0.7572 | −0.0094 | 0.9906 | 0.9859 |
| | A_G | | | | | | | | | |
| 4 | 4_38578411_ | 38578411 | 38576790 | rs7673092 | G | C | 0.1231 | 0.0062 | 1.0062 | 1.0015 |
| | C_G | | | | | | | | | |
| 4 | 4_38751752_ | 38751752 | 38750131 | rs4540059 | A | G | 0.1773 | 0.0050 | 1.0050 | 1.0018 |
| | G_A | | | | | | | | | |
| 4 | 4_38784633_ | 38784633 | 38783012 | rs10012017 | T | G | 0.2493 | 0.0175 | 1.0177 | 1.0088 |
| | G_T | | | | | | | | | |
| 4 | 4_38999972_ | 38999972 | 38998352 | rs112774343 | C | T | 0.2603 | 0.0006 | 1.0006 | 1.0003 |
| | T_C | | | | | | | | | |
| 4 | 4_39909408_ | 39909408 | 39907788 | rs62309184 | A | G | 0.2446 | 0.0044 | 1.0044 | 1.0022 |
| | G_A | | | | | | | | | |
| 4 | 4_43520590_ | 43520590 | 43518573 | rs4861284 | G | T | 0.9255 | −0.0065 | 0.9935 | 0.9880 |
| | T_G | | | | | | | | | |
| 4 | 4_43521885_ | 43521885 | 43519868 | rs34523991 | A | T | 0.8171 | −0.0064 | 0.9936 | 0.9896 |
| | T_A | | | | | | | | | |
| 4 | 4_45718837_ | 45718837 | 45716820 | rs148855431 | A | T | 0.0422 | 0.0004 | 1.0004 | 1.0000 |
| | T_A | | | | | | | | | |
| 4 | 4_45786423_ | 45786423 | 45784406 | rs114877756 | C | T | 0.0555 | 0.0199 | 1.0201 | 1.0022 |
| | T_C | | | | | | | | | |
| 4 | 4_47658534_ | 47658534 | 47656517 | rs6447578 | T | A | 0.4404 | 0.0013 | 1.0013 | 1.0011 |
| | A_T | | | | | | | | | |
| 4 | 4_48073073_ | 48073073 | 48071056 | rs62301688 | T | C | 0.2784 | −0.0066 | 0.9934 | 0.9963 |
| | C_T | | | | | | | | | |
| 4 | 4_48099439_ | 48099439 | 48097422 | rs4047946 | CTT | C | 0.6596 | 0.0003 | 1.0003 | 1.0004 |
| | C_CTT | | | | | | | | | |
| 4 | 4_53912593_ | 53912593 | 53046426 | rs12506630 | C | G | 0.3736 | −0.0115 | 0.9886 | 0.9915 |
| | G_C | | | | | | | | | |
| 4 | 4_55885742_ | 55885742 | 55019575 | rs71599662 | G | A | 0.1979 | 0.0025 | 1.0025 | 1.0010 |
| | A_G | | | | | | | | | |
| 4 | 4_55894238_ | 55894238 | 55028071 | rs13105577 | A | G | 0.1749 | 0.0106 | 1.0107 | 1.0037 |
| | G_A | | | | | | | | | |
| 4 | 4_56387771_ | 56387771 | 55521604 | rs114924440 | T | C | 0.0252 | 0.0325 | 1.0330 | 1.0017 |
| | C_T | | | | | | | | | |
| 4 | 4_57870386_ | 57870386 | 57004220 | 4:57870386_T | T | TTTC | 0.0438 | 0.0222 | 1.0224 | 1.0020 |
| | TTTCACTGTGT | | | TTCACTGTGTT | | ACTG | | | | |
| | TGGCCAGGCTG | | | GGCCAGGCTG | | TGTT | | | | |
| | GTC_T | | | GTC_T | | GGCC | | | | |
| | | | | | | AGGC | | | | |
| | | | | | | TGGT | | | | |
| | | | | | | C | | | | |
| 4 | 4_57879792_ | 57879792 | 57013626 | rs4865169 | T | C | 0.5223 | 0.0076 | 1.0076 | 1.0080 |
| | C_T | | | | | | | | | |
| 4 | 4_57910940_ | 57910940 | 57044774 | rs11937978 | A | G | 0.3279 | 0.0000 | 1.0000 | 1.0000 |
| | G_A | | | | | | | | | |
| 4 | 4_57935047_ | 57935047 | 57068881 | | CTTT | CTT | 0.1257 | −0.0032 | 0.9968 | 0.9992 |
| | CTT_CTTT | | | | | | | | | |
| 4 | 4_58002159_ | 58002159 | 57135993 | rs114175895 | T | C | 0.0130 | 0.0251 | 1.0254 | 1.0007 |
| | C_T | | | | | | | | | |
| 4 | 4_62017499_ | 62017499 | 61151781 | rs12508373 | A | G | 0.2554 | 0.0029 | 1.0029 | 1.0015 |
| | G_A | | | | | | | | | |
| 4 | 4_63347138_ | 63347138 | 62481420 | rs11735362 | T | G | 0.5269 | −0.0042 | 0.9958 | 0.9956 |
| | G_T | | | | | | | | | |
| 4 | 4_63354303_ | 63354303 | 62488585 | rs6843214 | G | A | 0.5877 | −0.0029 | 0.9971 | 0.9966 |
| | A_G | | | | | | | | | |
| 4 | 4_64303165_ | 64303165 | 63437447 | rs62303365 | C | T | 0.2643 | 0.0035 | 1.0035 | 1.0019 |
| | T_C | | | | | | | | | |
| 4 | 4_68081561_ | 68081561 | 67215843 | rs62315512 | A | G | 0.1865 | −0.0151 | 0.9850 | 0.9944 |
| | G_A | | | | | | | | | |
| 4 | 4_68292294_ | 68292294 | 67426576 | rs148825788 | G | A | 0.0821 | −0.0122 | 0.9879 | 0.9980 |
| | A_G | | | | | | | | | |
| 4 | 4_70336172_ | 70336172 | 69470454 | rs2736520 | C | T | 0.1498 | −0.0121 | 0.9880 | 0.9964 |
| | T_C | | | | | | | | | |
| 4 | 4_73752735_ | 73752735 | 72887018 | rs76886866 | A | T | 0.0180 | −0.0141 | 0.9860 | 0.9995 |
| | T_A | | | | | | | | | |
| 4 | 4_74222782_ | 74222782 | 73357065 | rs55881006 | A | G | 0.0333 | −0.0195 | 0.9807 | 0.9987 |
| | G_A | | | | | | | | | |
| 4 | 4_74784651_ | 74784651 | 73918934 | 4:74784651_G | G | GA | 0.0546 | 0.0292 | 1.0296 | 1.0032 |
| | GA_G | | | A_G | | | | | | |
| 4 | 4_77057905_ | 77057905 | 76136752 | rs4376175 | T | G | 0.1579 | −0.0143 | 0.9858 | 0.9955 |
| | G_T | | | | | | | | | |
| 4 | 4_78073483_ | 78073483 | 77152330 | rs6849124 | G | A | 0.5087 | −0.0001 | 0.9999 | 0.9999 |
| | A_G | | | | | | | | | |
| 4 | 4_78121478_ | 78121478 | 77200325 | rs4582179 | C | G | 0.4848 | −0.0100 | 0.9900 | 0.9904 |
| | G_C | | | | | | | | | |
| 4 | 4_83892103_ | 83892103 | 82970950 | rs756255559 | C | CTGA | 0.5671 | −0.0016 | 0.9984 | 0.9982 |
| | CTGATT_C | | | | | TT | | | | |
| 4 | 4_84370124_ | 84370124 | 83448971 | rs774021038 | TA | TAA | 0.5324 | −0.0176 | 0.9826 | 0.9815 |
| | TAA_TA | | | | | | | | | |
| 4 | 4_84405745_ | 84405745 | 83484592 | rs6844460 | C | T | 0.4946 | −0.0071 | 0.9929 | 0.9930 |
| | T_C | | | | | | | | | |
| 4 | 4_84876371_ | 84876371 | 83955218 | rs57484869 | C | A | 0.2697 | −0.0025 | 0.9975 | 0.9987 |
| | A_C | | | | | | | | | |
| 4 | 4_86501825_ | 86501825 | 85580672 | rs1161259 | T | A | 0.2594 | 0.0011 | 1.0011 | 1.0006 |
| | A_T | | | | | | | | | |
| 4 | 4_86577565_ | 86577565 | 85656412 | rs2869360 | C | T | 0.2907 | 0.0103 | 1.0104 | 1.0060 |
| | T_C | | | | | | | | | |
| 4 | 4_86616140_ | 86616140 | 85694987 | rs71599422 | T | C | 0.0462 | −0.0112 | 0.9889 | 0.9990 |
| | C_T | | | | | | | | | |
| 4 | 4_87225701_ | 87225701 | 86304548 | rs13132706 | A | G | 0.2868 | −0.0076 | 0.9924 | 0.9957 |
| | G_A | | | | | | | | | |
| 4 | 4_87272549_ | 87272549 | 86351396 | rs201445110 | A | AACA | 0.5038 | 0.0015 | 1.0015 | 1.0015 |
| | AACAC_A | | | | | C | | | | |
| 4 | 4_88799710_ | 88799710 | 87878558 | rs1463104 | C | T | 0.3454 | 0.0039 | 1.0039 | 1.0027 |
| | T_C | | | | | | | | | |
| 4 | 4_88809797_ | 88809797 | 87888645 | | G | A | 0.7973 | −0.0027 | 0.9973 | 0.9957 |
| | A_G | | | | | | | | | |
| 4 | 4_88979529_ | 88979529 | 88058377 | rs2725207 | A | C | 0.1412 | 0.0000 | 1.0000 | 1.0000 |
| | C_A | | | | | | | | | |
| 4 | 4_89023224_ | 89023224 | 88102072 | rs4693924 | A | G | 0.0932 | −0.0054 | 0.9946 | 0.9990 |
| | G_A | | | | | | | | | |
| 4 | 4_89240476_ | 89240476 | 88319324 | rs17014016 | A | G | 0.4395 | 0.0145 | 1.0146 | 1.0129 |
| | G_A | | | | | | | | | |
| 4 | 4_89246214_ | 89246214 | 88325062 | rs10516806 | T | G | 0.5286 | −0.0005 | 0.9995 | 0.9995 |
| | G_T | | | | | | | | | |
| 4 | 4_89831258_ | 89831258 | 88910107 | rs76243762 | G | A | 0.0703 | 0.0125 | 1.0126 | 1.0018 |
| | A_G | | | | | | | | | |
| 4 | 4_89931611_ | 89931611 | 89010460 | rs72665870 | G | A | 0.0278 | −0.0214 | 0.9788 | 0.9988 |
| | A_G | | | | | | | | | |
| 4 | 4_90012134_ | 90012134 | 89090983 | rs13140834 | T | C | 0.1861 | 0.0006 | 1.0006 | 1.0002 |
| | C_T | | | | | | | | | |
| 4 | 4_91026587_ | 91026587 | 90105436 | rs114957953 | G | A | 0.0363 | 0.0104 | 1.0105 | 1.0008 |
| | A_G | | | | | | | | | |
| 4 | 4_91237099_ | 91237099 | 90315948 | rs57992896 | C | G | 0.3138 | 0.0014 | 1.0014 | 1.0009 |
| | G_C | | | | | | | | | |
| 4 | 4_91611823_ | 91611823 | 90690672 | rs17017396 | G | A | 0.1908 | 0.0161 | 1.0162 | 1.0062 |
| | A_G | | | | | | | | | |
| 4 | 4_91836876_ | 91836876 | 90915725 | | GT | G | 0.4405 | −0.0017 | 0.9983 | 0.9985 |
| | G_GT | | | | | | | | | |
| 4 | 4_92403085_ | 92403085 | 91481934 | 4:92403085_A | A | AAC | 0.0151 | 0.0178 | 1.0180 | 1.0005 |
| | AAC_A | | | AC_A | | | | | | |
| 4 | 4_92546996_ | 92546996 | 91625845 | rs145893239 | C | A | 0.4554 | −0.0068 | 0.9932 | 0.9938 |
| | A_C | | | | | | | | | |
| 4 | 4_92595386_ | 92595386 | 91674235 | rs7664260 | T | C | 0.4696 | −0.0061 | 0.9939 | 0.9943 |
| | C_T | | | | | | | | | |
| 4 | 4_92638157_ | 92638157 | 91717006 | rs9307101 | T | C | 0.3757 | 0.0016 | 1.0016 | 1.0012 |
| | C_T | | | | | | | | | |
| 4 | 4_95147658_ | 95147658 | 94226507 | 4:95147658_A | A | ATT | 0.5661 | −0.0031 | 0.9969 | 0.9965 |
| | ATT_A | | | TT_A | | | | | | |
| 4 | 4_95877957_ | 95877957 | 94956806 | rs3839108 | AT | A | 0.4614 | 0.0050 | 1.0050 | 1.0046 |
| | A_AT | | | | | | | | | |
| 4 | 4_98651479_ | 98651479 | 97730328 | rs9991112 | C | A | 0.4565 | 0.0020 | 1.0020 | 1.0018 |
| | A_C | | | | | | | | | |
| 4 | 4_99077721_ | 99077721 | 98156570 | rs12508039 | G | A | 0.0352 | 0.0061 | 1.0061 | 1.0004 |
| | A_G | | | | | | | | | |
| 4 | 4_99103707_ | 99103707 | 98182556 | rs773219786 | G | GA | 0.3080 | −0.0079 | 0.9921 | 0.9952 |
| | GA_G | | | | | | | | | |
| 4 | 4_99428163_ | 99428163 | 98507012 | rs955931 | T | C | 0.1535 | −0.0045 | 0.9955 | 0.9986 |
| | C_T | | | | | | | | | |
| 4 | 4_99922538_ | 99922538 | 99001387 | rs111953321 | A | G | 0.0369 | 0.0079 | 1.0079 | 1.0006 |
| | G_A | | | | | | | | | |
| 4 | 4_100017829_ | 100017829 | 99096678 | rs113772463 | T | C | 0.0422 | −0.0063 | 0.9937 | 0.9995 |
| | C_T | | | | | | | | | |
| 4 | 4_100071687_ | 100071687 | 99150530 | rs6850191 | C | T | 0.8141 | 0.0016 | 1.0016 | 1.0026 |
| | T_C | | | | | | | | | |
| 4 | 4_100293109_ | 100293109 | 99371952 | rs62305782 | T | C | 0.1138 | 0.0100 | 1.0101 | 1.0023 |
| | C_T | | | | | | | | | |
| 4 | 4_100324473_ | 100324473 | 99403316 | rs34096752 | T | A | 0.0614 | 0.0172 | 1.0173 | 1.0021 |
| | A_T | | | | | | | | | |
| 4 | 4_101448359_ | 101448359 | 100527202 | rs72692173 | G | A | 0.0574 | −0.0101 | 0.9900 | 0.9988 |
| | A_G | | | | | | | | | |
| 4 | 4_101542512_ | 101542512 | 100621355 | rs72694380 | G | T | 0.0447 | −0.0285 | 0.9719 | 0.9975 |
| | T_G | | | | | | | | | |
| 4 | 4_104371655_ | 104371655 | 103450498 | rs10016947 | C | T | 0.0571 | −0.0209 | 0.9793 | 0.9976 |
| | T_C | | | | | | | | | |
| 4 | 4_106024327_ | 106024327 | 105103170 | 4:106024327_ | C | CT | 0.0690 | 0.0000 | 1.0000 | 1.0000 |
| | CT_C | | | CT_C | | | | | | |
| 4 | 4_106042993_ | 106042993 | 105121836 | rs35457521 | TA | T | 0.4296 | 0.0002 | 1.0002 | 1.0002 |
| | T_TA | | | | | | | | | |
| 4 | 4_106044481_ | 106044481 | 105123324 | rs62331127 | T | C | 0.2972 | 0.0002 | 1.0002 | 1.0001 |
| | C_T | | | | | | | | | |
| 4 | 4_106069013_ | 106069013 | 105147856 | rs62331150 | T | G | 0.2289 | 0.0227 | 1.0230 | 1.0105 |
| | G_T | | | | | | | | | |
| 4 | 4_106094427_ | 106094427 | 105173270 | rs2903385 | A | G | 0.4950 | 0.0000 | 1.0000 | 1.0000 |
| | G_A | | | | | | | | | |
| 4 | 4_106356761_ | 106356761 | 105435604 | rs77928427A | | C | 0.2394 | 0.0174 | 1.0176 | 1.0084 |
| | C_A | | | | | | | | | |
| 4 | 4_111105528_ | 111105528 | 110184372 | rs79136024 | G | T | 0.0506 | 0.0129 | 1.0130 | 1.0013 |
| | T_G | | | | | | | | | |
| 4 | 4_113081560_ | 113081560 | 112160404 | rs34894300 | CA | C | 0.3420 | −0.0095 | 0.9905 | 0.9935 |
| | C_CA | | | | | | | | | |
| 4 | 4_113109702_ | 113109702 | 112188546 | rs1045940 | G | T | 0.4511 | −0.0033 | 0.9967 | 0.9970 |
| | T_G | | | | | | | | | |
| 4 | 4_114703466_ | 114703466 | 113782310 | rs112709257 | TA | T | 0.1913 | −0.0029 | 0.9971 | 0.9989 |
| | T_TA | | | | | | | | | |
| 4 | 4_118605518_ | 118605518 | 117684363 | rs2389389 | T | A | 0.0722 | 0.0000 | 1.0000 | 1.0000 |
| | A_T | | | | | | | | | |
| 4 | 4_118701529_ | 118701529 | 117780374 | rs28945124 | A | G | 0.0343 | −0.0288 | 0.9716 | 0.9981 |
| | G_A | | | | | | | | | |
| 4 | 4_121422421_ | 121422421 | 120501266 | rs72672563 | G | A | 0.0855 | −0.0070 | 0.9930 | 0.9988 |
| | A_G | | | | | | | | | |
| 4 | 4_122232705_ | 122232705 | 121311550 | rs9992405 | G | A | 0.0685 | −0.0137 | 0.9864 | 0.9981 |
| | A_G | | | | | | | | | |
| 4 | 4_122725303_ | 122725303 | 121804148 | rs6830443 | A | G | 0.3797 | 0.0066 | 1.0066 | 1.0050 |
| | G_A | | | | | | | | | |
| 4 | 4_123612272_ | 123612272 | 122691117 | rs13103403 | G | C | 0.2376 | 0.0049 | 1.0049 | 1.0023 |
| | C_G | | | | | | | | | |
| 4 | 4_125108130_ | 125108130 | 124186975 | rs10007609 | T | G | 0.5310 | −0.0095 | 0.9905 | 0.9900 |
| | G_T | | | | | | | | | |
| 4 | 4_125173747_ | 125173747 | 124252592 | rs7657011 | T | C | 0.4276 | −0.0009 | 0.9991 | 0.9992 |
| | C_T | | | | | | | | | |
| 4 | 4_126202299_ | 126202299 | 125281144 | rs57474540 | G | A | 0.2946 | −0.0043 | 0.9957 | 0.9975 |
| | A_G | | | | | | | | | |
| 4 | 4_126292075_ | 126292075 | 125370920 | rs62321344 | T | C | 0.1911 | 0.0102 | 1.0103 | 1.0039 |
| | C_T | | | | | | | | | |
| 4 | 4_126654773_ | 126654773 | 125733618 | rs9307576 | T | G | 0.4626 | 0.0046 | 1.0046 | 1.0043 |
| | G_T | | | | | | | | | |
| 4 | 4_126708773_ | 126708773 | 125787618 | rs5861742 | TA | T | 0.5477 | 0.0054 | 1.0054 | 1.0059 |
| | T_TA | | | | | | | | | |
| 4 | 4_126752992_ | 126752992 | 125831837 | | AAT | A | 0.5167 | −0.0018 | 0.9982 | 0.9981 |
| | A_AAT | | | | | | | | | |
| 4 | 4_126771475_ | 126771475 | 125850320 | rs1506162 | A | G | 0.8163 | 0.0032 | 1.0032 | 1.0052 |
| | G_A | | | | | | | | | |
| 4 | 4_126835376_ | 126835376 | 125914221 | rs4525994 | A | G | 0.5964 | −0.0174 | 0.9828 | 0.9795 |
| | G_A | | | | | | | | | |
| 4 | 4_126843504_ | 126843504 | 125922349 | rs77528541 | T | G | 0.1348 | −0.0131 | 0.9870 | 0.9965 |
| | G_T | | | | | | | | | |
| 4 | 4_126934050_ | 126934050 | 126012895 | rs62314116 | G | A | 0.3328 | 0.0000 | 1.0000 | 1.0000 |
| | A_G | | | | | | | | | |
| 4 | 4_127515501_ | 127515501 | 126594346 | rs313107 | T | A | 0.0939 | 0.0097 | 1.0097 | 1.0018 |
| | A_T | | | | | | | | | |
| 4 | 4_127537947_ | 127537947 | 126616792 | rs1994679 | A | C | 0.6093 | −0.0003 | 0.9997 | 0.9996 |
| | C_A | | | | | | | | | |
| 4 | 4_130558257_ | 130558257 | 129637102 | rs6852416 | T | G | 0.0851 | 0.0102 | 1.0103 | 1.0017 |
| | G_T | | | | | | | | | |
| 4 | 4_132497181_ | 132497181 | 131576026 | rs745490368 | G | GAGA | 0.0781 | 0.0148 | 1.0149 | 1.0023 |
| | GAGA_G | | | | | | | | | |
| 4 | 4_137743220_ | 137743220 | 136822066 | rs4455476 | A | C | 0.1742 | 0.0021 | 1.0021 | 1.0007 |
| | C_A | | | | | | | | | |
| 4 | 4_143343605_ | 143343605 | 142422452 | rs6820463 | T | C | 0.3058 | 0.0060 | 1.0060 | 1.0037 |
| | C_T | | | | | | | | | |
| 4 | 4_143467195_ | 143467195 | 142546042 | rs56039025 | T | C | 0.1115 | −0.0311 | 0.9694 | 0.9932 |
| | C_T | | | | | | | | | |
| 4 | 4_144406937_ | 144406937 | 143485784 | rs17017812 | G | A | 0.2279 | 0.0057 | 1.0057 | 1.0026 |
| | A_G | | | | | | | | | |
| 4 | 4_146731529_ | 146731529 | 145810377 | rs59831926 | C | T | 0.0985 | −0.0209 | 0.9793 | 0.9959 |
| | T_C | | | | | | | | | |
| 4 | 4_147079249_ | 147079249 | 146158097 | rs72727929 | C | T | 0.0578 | 0.0173 | 1.0175 | 1.0020 |
| | T_C | | | | | | | | | |
| 4 | 4_149121794_ | 149121794 | 148200643 | rs6854045 | C | T | 0.2632 | 0.0021 | 1.0021 | 1.0011 |
| | T_C | | | | | | | | | |
| 4 | 4_149134380_ | 149134380 | 148213229 | rs6857487 | A | T | 0.4317 | 0.0038 | 1.0038 | 1.0033 |
| | T_A | | | | | | | | | |
| 4 | 4_151144895_ | 151144895 | 150223743 | rs140778874 | CAATA | C | 0.6647 | 0.0000 | 1.0000 | 1.0000 |
| | C_CAATA | | | | | | | | | |
| 4 | 4_151218296_ | 151218296 | 150297144 | rs745707748 | C | CATA | 0.6533 | 0.0027 | 1.0027 | 1.0035 |
| | CATATTT_C | | | | | TTT | | | | |
| 4 | 4_151268153_ | 151268153 | 150347001 | rs59975091 | GTA | G | 0.2841 | −0.0042 | 0.9958 | 0.9976 |
| | G_GTA | | | | | | | | | |
| 4 | 4_151271452_ | 151271452 | 150350300 | rs13145548 | G | A | 0.6396 | 0.0019 | 1.0019 | 1.0024 |
| | A_G | | | | | | | | | |
| 4 | 4_151281390_ | 151281390 | 150360238 | rs13128588 | T | C | 0.3713 | 0.0055 | 1.0055 | 1.0041 |
| | C_T | | | | | | | | | |
| 4 | 4_151296384_ | 151296384 | 150375232 | rs17504352 | T | G | 0.2950 | −0.0017 | 0.9983 | 0.9990 |
| | G_T | | | | | | | | | |
| 4 | 4_161993005_ | 161993005 | 161071853 | rs930402 | T | C | 0.3112 | 0.0038 | 1.0038 | 1.0024 |
| | C_T | | | | | | | | | |
| 4 | 4_163461356_ | 163461356 | 162540204 | rs113196123 | G | A | 0.0544 | 0.0130 | 1.0131 | 1.0014 |
| | A_G | | | | | | | | | |
| 4 | 4_166052580_ | 166052580 | 165131428 | rs17585041 | T | C | 0.0679 | 0.0202 | 1.0204 | 1.0028 |
| | C_T | | | | | | | | | |
| 4 | 4_166313062_ | 166313062 | 165391910 | rs17046389 | G | A | 0.1487 | −0.0133 | 0.9868 | 0.9961 |
| | A_G | | | | | | | | | |
| 4 | 4_166346355_ | 166346355 | 165425203 | rs28846814 | C | G | 0.1476 | −0.0007 | 0.9993 | 0.9998 |
| | G_C | | | | | | | | | |
| 4 | 4_166588797_ | 166588797 | 165667645 | rs5863769 | TA | T | 0.2715 | −0.0017 | 0.9983 | 0.9991 |
| | T_TA | | | | | | | | | |
| 4 | 4_166591321_ | 166591321 | 165670169 | rs2714807 | C | T | 0.4401 | −0.0046 | 0.9954 | 0.9960 |
| | T_C | | | | | | | | | |
| 4 | 4_169004537_ | 169004537 | 168083386 | 4:169004537_ | C | CAA | 0.5367 | 0.0039 | 1.0039 | 1.0042 |
| | CAA_C | | | CAA_C | | | | | | |
| 4 | 4_171493439_ | 171493439 | 170572288 | rs415366 | T | C | 0.6642 | −0.0010 | 0.9990 | 0.9987 |
| | C_T | | | | | | | | | |
| 4 | 4_171536786_ | 171536786 | 170615635 | rs1596251 | T | C | 0.4213 | 0.0006 | 1.0006 | 1.0005 |
| | C_T | | | | | | | | | |
| 4 | 4_171943596_ | 171943596 | 171022445 | rs139966312 | C | G | 0.0315 | 0.0054 | 1.0054 | 1.0003 |
| | G_C | | | | | | | | | |
| 4 | 4_172626489_ | 172626489 | 171705338 | rs1565468 | T | C | 0.1069 | −0.0008 | 0.9992 | 0.9998 |
| | C_T | | | | | | | | | |
| 4 | 4_174741894_ | 174741894 | 173820743 | rs4469049 | C | T | 0.1972 | 0.0070 | 1.0070 | 1.0028 |
| | T_C | | | | | | | | | |
| 4 | 4_175716260_ | 175716260 | 174795109 | rs6830584 | T | C | 0.9267 | 0.0070 | 1.0070 | 1.0131 |
| | C_T | | | | | | | | | |
| 4 | 4_175836048_ | 175836048 | 174914897 | rs6553847 | A | G | 0.5730 | −0.0115 | 0.9886 | 0.9869 |
| | G_A | | | | | | | | | |
| 4 | 4_175842495_ | 175842495 | 174921344 | rs28436676 | A | G | 0.1161 | −0.0281 | 0.9723 | 0.9936 |
| | G_A | | | | | | | | | |
| 4 | 4_175844668_ | 175844668 | 174923517 | 4:175844668_ | C | CATT | 0.0919 | −0.0383 | 0.9624 | 0.9931 |
| | CATTAT_C | | | CATTAT_C | | AT | | | | |
| 4 | 4_175847436_ | 175847436 | 174926285 | rs62334414 | A | C | 0.3433 | 0.0129 | 1.0130 | 1.0089 |
| | C_A | | | | | | | | | |
| 4 | 4_175857997_ | 175857997 | 174936846 | rs62334421 | T | A | 0.3490 | 0.0000 | 1.0000 | 1.0000 |
| | A_T | | | | | | | | | |
| 4 | 4_176820922_ | 176820922 | 175899771 | rs34037910 | CA | C | 0.2762 | −0.0050 | 0.9950 | 0.9972 |
| | C_CA | | | | | | | | | |
| 4 | 4_180659077_ | 180659077 | 179737924 | rs112038385 | G | A | 0.0291 | −0.0134 | 0.9867 | 0.9992 |
| | A_G | | | | | | | | | |
| 4 | 4_182435039_ | 182435039 | 181513886 | rs6816262 | A | G | 0.0322 | 0.0000 | 1.0000 | 1.0000 |
| | G_A | | | | | | | | | |
| 4 | 4_182543945_ | 182543945 | 181622792 | rs10002408 | C | T | 0.9179 | −0.0109 | 0.9892 | 0.9802 |
| | T_C | | | | | | | | | |
| 4 | 4_182602168_ | 182602168 | 181681015 | rs75399632 | C | A | 0.0765 | −0.0107 | 0.9894 | 0.9984 |
| | A_C | | | | | | | | | |
| 4 | 4_184355335_ | 184355335 | 183434182 | rs181139981 | C | G | 0.0310 | −0.0300 | 0.9704 | 0.9982 |
| | G_C | | | | | | | | | |
| 4 | 4_184357607_ | 184357607 | 183436454 | rs78515438 | A | G | 0.0837 | 0.0038 | 1.0038 | 1.0006 |
| | G_A | | | | | | | | | |
| 4 | 4_185259670_ | 185259670 | 184338517 | rs4269220 | G | T | 0.4643 | 0.0043 | 1.0043 | 1.0040 |
| | T_G | | | | | | | | | |
| 4 | 4 185260558_ | 185260558 | 184339405 | rs34156200 | CA | C | 0.6514 | 0.0029 | 1.0029 | 1.0038 |
| | C_CA | | | | | | | | | |
| 4 | 4_186838211_ | 186838211 | 185917057 | rs7671869 | C | A | 0.4070 | −0.0030 | 0.9970 | 0.9976 |
| | A_C | | | | | | | | | |
| 4 | 4_186847709_ | 186847709 | 185926555 | rs11733388 | T | G | 0.3179 | −0.0033 | 0.9967 | 0.9979 |
| | G_T | | | | | | | | | |
| 4 | 4_187503758_ | 187503758 | 186582604 | | T | A | 0.4471 | 0.0094 | 1.0094 | 1.0085 |
| | A_T | | | | | | | | | |
| 4 | 4_187511304_ | 187511304 | 186590150 | rs11410853 | GT | G | 0.4433 | 0.0032 | 1.0032 | 1.0028 |
| | G_GT | | | | | | | | | |
| 4 | 4_189136747_ | 189136747 | 188215593 | rs6833483 | C | T | 0.8191 | −0.0091 | 0.9909 | 0.9852 |
| | T_C | | | | | | | | | |
| 4 | 4_189165592_ | 189165592 | 188244438 | rs111992262 | CT | C | 0.6126 | 0.0040 | 1.0040 | 1.0049 |
| | C_CT | | | | | | | | | |
| 4 | 4_189200025_ | 189200025 | 188278871 | 4:189200025 | T | TAC | 0.8092 | 0.0167 | 1.0168 | 1.0274 |
| | TAC_T | | | TAC_T | | | | | | |
| 4 | 4_189206717_ | 189206717 | 188285563 | rs4132198 | T | C | 0.6448 | −0.0022 | 0.9978 | 0.9972 |
| | C_T | | | | | | | | | |
| 4 | 4_189227886_ | 189227886 | 188306732 | 4:189227886 | C | CAA | 0.3190 | 0.0064 | 1.0064 | 1.0041 |
| | CAA_C | | | CAA C | | | | | | |
| 4 | 4_189326401_ | 189326401 | 188405247 | rs372860528 | T | C | 0.0206 | −0.0167 | 0.9834 | 0.9993 |
| | C_T | | | | | | | | | |
| 4 | 4_190097328_ | 190097328 | 189176174 | rs62340381 | G | A | 0.0421 | 0.0121 | 1.0122 | 1.0010 |
| | A_G | | | | | | | | | |
| 5 | 5_345109_ | 345109 | 344994 | rs62641919 | C | T | 0.0544 | 0.0291 | 1.0295 | 1.0032 |
| | T_C | | | | | | | | | |
| 5 | 5_457261_ | 457261 | 457146 | rs11749327 | A | C | 0.2846 | −0.0047 | 0.9953 | 0.9973 |
| | C_A | | | | | | | | | |
| 5 | 5_477719_ | 477719 | 477604 | rs13356700 | G | A | 0.5029 | −0.0026 | 0.9974 | 0.9974 |
| | A_G | | | | | | | | | |
| 5 | 5_1153459_ | 1153459 | 1153344 | rs74510663 | T | C | 0.0372 | 0.0000 | 1.0000 | 1.0000 |
| | C_T | | | | | | | | | |
| 5 | 5_1168879_ | 1168879 | 1168764 | rs140709218 | C | T | 0.0401 | 0.0142 | 1.0143 | 1.0011 |
| | T_C | | | | | | | | | |
| 5 | 5_1206479_ | 1206479 | 1206364 | rs112616037 | CTG | C | 0.1050 | 0.0127 | 1.0128 | 1.0027 |
| | C_CTG | | | | | | | | | |
| 5 | 5_1209436_ | 1209436 | 1209321 | rs79784189 | G | A | 0.1213 | 0.0119 | 1.0120 | 1.0029 |
| | A_G | | | | | | | | | |
| 5 | 5_1218711_ | 1218711 | 1218596 | rs12332680 | G | A | 0.3134 | −0.0038 | 0.9962 | 0.9976 |
| | A_G | | | | | | | | | |
| 5 | 5_1275857_ | 1275857 | 1275742 | rs13156167 | C | T | 0.1558 | 0.0000 | 1.0000 | 1.0000 |
| | T_C | | | | | | | | | |
| 5 | 5_1279790_ | 1279790 | 1279675 | rs10069690 | T | C | 0.2592 | 0.0245 | 1.0248 | 1.0129 |
| | C_T | | | | | | | | | |
| 5 | 5_1280028_ | 1280028 | 1279913 | rs2242652 | A | G | 0.1979 | 0.0000 | 1.0000 | 1.0000 |
| | G_A | | | | | | | | | |
| 5 | 5_1280128_ | 1280128 | 1280013 | rs7734992 | C | T | 0.4243 | 0.0012 | 1.0012 | 1.0010 |
| | T_C | | | | | | | | | |
| 5 | 5_1285974_ | 1285974 | 1285859 | rs7705526 | A | C | 0.3333 | 0.0068 | 1.0068 | 1.0046 |
| | C_A | | | | | | | | | |
| 5 | 5_1294086_ | 1294086 | 1293971 | rs2736098 | T | C | 0.2656 | −0.0058 | 0.9942 | 0.9969 |
| | C_T | | | | | | | | | |
| 5 | 5_1296255_ | 1296255 | 1296140 | rs3215401 | AG | A | 0.3072 | −0.0162 | 0.9839 | 0.9901 |
| | A_AG | | | | | | | | | |
| 5 | 5_1297258_ | 1297258 | 1297143 | rs6554754 | C | T | 0.8667 | −0.0054 | 0.9946 | 0.9907 |
| | T_C | | | | | | | | | |
| 5 | 5_1297854_ | 1297854 | 1297739 | rs2736107 | T | C | 0.2827 | −0.0054 | 0.9946 | 0.9970 |
| | C_T | | | | | | | | | |
| 5 | 5_1297918_ | 1297918 | 1297803 | rs7449190 | C | T | 0.8489 | −0.0022 | 0.9978 | 0.9963 |
| | T_C | | | | | | | | | |
| 5 | 5_1299379_ | 1299379 | 1299264 | rs2735846 | G | C | 0.5049 | −0.0160 | 0.9841 | 0.9840 |
| | C_G | | | | | | | | | |
| 5 | 5_1318797_ | 1318797 | 1318682 | rs148487301 | C | T | 0.0236 | 0.0000 | 1.0000 | 1.0000 |
| | T_C | | | | | | | | | |
| 5 | 5_1353077_ | 1353077 | 1352962 | rs62329727 | C | T | 0.0121 | 0.0393 | 1.0401 | 1.0010 |
| | T_C | | | | | | | | | |
| 5 | 5_1442521_ | 1442521 | 1442406 | rs2617605 | C | T | 0.3602 | −0.0004 | 0.9996 | 0.9997 |
| | T_C | | | | | | | | | |
| 5 | 5_1453344_ | 1453344 | 1453229 | rs2617583 | A | C | 0.5534 | −0.0097 | 0.9903 | 0.9893 |
| | C_A | | | | | | | | | |
| 5 | 5_2755932_ | 2755932 | 2755818 | rs4866667 | T | C | 0.6730 | −0.0077 | 0.9923 | 0.9897 |
| | C_T | | | | | | | | | |
| 5 | 5_2769043_ | 2769043 | 2768929 | rs6875050 | G | T | 0.3626 | −0.0039 | 0.9961 | 0.9972 |
| | T_G | | | | | | | | | |
| 5 | 5_2777029_ | 2777029 | 2776915 | rs4866496 | A | G | 0.4139 | 0.0020 | 1.0020 | 1.0017 |
| | G_A | | | | | | | | | |
| 5 | 5_2777869_ | 2777869 | 2777755 | rs11960374 | C | T | 0.3775 | 0.0062 | 1.0062 | 1.0047 |
| | T_C | | | | | | | | | |
| 5 | 5_3892571_ | 3892571 | 3892457 | rs534632286 | TG | T | 0.5948 | 0.0090 | 1.0090 | 1.0108 |
| | T_TG | | | | | | | | | |
| 5 | 5_4106597_ | 4106597 | 4106484 | rs115682874 | T | C | 0.0317 | −0.0058 | 0.9942 | 0.9996 |
| | C_T | | | | | | | | | |
| 5 | 5_5308256_ | 5308256 | 5308143 | rs35614306 | T | C | 0.0827 | 0.0158 | 1.0159 | 1.0026 |
| | C_T | | | | | | | | | |
| 5 | 5_5311838_ | 5311838 | 5311725 | rs70965937 | GT | G | 0.0837 | 0.0000 | 1.0000 | 1.0000 |
| | G_GT | | | | | | | | | |
| 5 | 5_6574023_ | 6574023 | 6573910 | rs35589606 | CA | C | 0.1240 | 0.0082 | 1.0082 | 1.0020 |
| | C_CA | | | | | | | | | |
| 5 | 5_6613517_ | 6613517 | 6613404 | rs3756426 | T | C | 0.1968 | 0.0083 | 1.0083 | 1.0033 |
| | C_T | | | | | | | | | |
| 5 | 5_6677699_ | 6677699 | 6677586 | rs13166363 | A | G | 0.2734 | −0.0044 | 0.9956 | 0.9976 |
| | G_A | | | | | | | | | |
| 5 | 5_7225615_ | 7225615 | 7225502 | rs116151459 | G | A | 0.0336 | 0.0104 | 1.0105 | 1.0007 |
| | A_G | | | | | | | | | |
| 5 | 5_14320462_ | 14320462 | 14320353 | rs73059540 | C | G | 0.0731 | 0.0065 | 1.0065 | 1.0010 |
| | G_C | | | | | | | | | |
| 5 | 5_14365895_ | 14365895 | 14365786 | rs43171 | G | A | 0.5129 | −0.0043 | 0.9957 | 0.9956 |
| | A_G | | | | | | | | | |
| 5 | 5_14380773_ | 14380773 | 14380664 | rs247136 | C | T | 0.1727 | 0.0039 | 1.0039 | 1.0014 |
| | T_C | | | | | | | | | |
| 5 | 5_14403252_ | 14403252 | 14403143 | 5:14403252_T | T | TGTG | 0.2617 | 0.0046 | 1.0046 | 1.0024 |
| | TGTG_T | | | GTG_T | | | | | | |
| 5 | 5_15113644_ | 15113644 | 15113535 | | CTG | C | 0.1534 | −0.0121 | 0.9880 | 0.9963 |
| | C_CTG | | | | | | | | | |
| 5 | 5_16231194_ | 16231194 | 16231085 | rs17611291 | C | G | 0.5594 | −0.0088 | 0.9912 | 0.9902 |
| | G_C | | | | | | | | | |
| 5 | 5_16254310_ | 16254310 | 16254201 | rs11133838 | G | A | 0.5814 | −0.0130 | 0.9871 | 0.9850 |
| | A_G | | | | | | | | | |
| 5 | 5_21605146_ | 21605146 | 21605037 | rs12520587 | A | G | 0.6025 | 0.0062 | 1.0062 | 1.0075 |
| | G_A | | | | | | | | | |
| 5 | 5_22123627_ | 22123627 | 22123518 | rs62349111 | C | G | 0.0524 | 0.0065 | 1.0065 | 1.0007 |
| | G_C | | | | | | | | | |
| 5 | 5_25664626_ | 25664626 | 25664517 | rs35786359 | GA | G | 0.5266 | −0.0046 | 0.9954 | 0.9952 |
| | G_GA | | | | | | | | | |
| 5 | 5_25879426_ | 25879426 | 25879317 | rs10942144 | G | A | 0.1715 | −0.0139 | 0.9862 | 0.9953 |
| | A_G | | | | | | | | | |
| 5 | 5_25889346_ | 25889346 | 25889237 | rs1366468 | C | T | 0.3980 | 0.0004 | 1.0004 | 1.0003 |
| | T_C | | | | | | | | | |
| 5 | 5_27043424_ | 27043424 | 27043317 | rs77344846 | T | C | 0.0220 | −0.0089 | 0.9911 | 0.9996 |
| | C_T | | | | | | | | | |
| 5 | 5_27479118_ | 27479118 | 27479011 | rs193173568 | G | A | 0.1340 | −0.0163 | 0.9838 | 0.9957 |
| | A_G | | | | | | | | | |
| 5 | 5_27575812_ | 27575812 | 27575705 | | TAA | TA | 0.1006 | 0.0073 | 1.0073 | 1.0015 |
| | TA_TAA | | | | | | | | | |
| 5 | 5_28288287_ | 28288287 | 28288180 | rs2468479 | A | G | 0.1037 | 0.0066 | 1.0066 | 1.0014 |
| | G_A | | | | | | | | | |
| 5 | 5_31271073_ | 31271073 | 31270966 | rs11955971 | A | G | 0.3547 | −0.0103 | 0.9898 | 0.9927 |
| | G_A | | | | | | | | | |
| 5 | 5_31360086_ | 31360086 | 31359979 | rs334589 | T | C | 0.9022 | −0.0134 | 0.9867 | 0.9761 |
| | C_T | | | | | | | | | |
| 5 | 5_32579616_ | 32579616 | 32579510 | rs770436441 | T | TCA | 0.4844 | 0.0106 | 1.0107 | 1.0104 |
| | TCA_T | | | | | | | | | |
| 5 | 5_32641237_ | 32641237 | 32641131 | rs818132 | T | C | 0.4891 | 0.0050 | 1.0050 | 1.0049 |
| | C_T | | | | | | | | | |
| 5 | 5_35652823_ | 35652823 | 35652721 | rs16902377 | A | G | 0.4071 | −0.0068 | 0.9932 | 0.9945 |
| | G_A | | | | | | | | | |
| 5 | 5_35733285_ | 35733285 | 35733183 | rs4524534 | C | T | 0.6405 | 0.0125 | 1.0126 | 1.0162 |
| | T_C | | | | | | | | | |
| 5 | 5_36391846_ | 36391846 | 36391744 | 5:36391846_A | A | ATTC | 0.1480 | 0.0000 | 1.0000 | 1.0000 |
| | ATTCT_A | | | TTCT_A | | T | | | | |
| 5 | 5_38656341_ | 38656341 | 38656239 | rs10512690 | A | T | 0.4490 | 0.0120 | 1.0121 | 1.0109 |
| | T_A | | | | | | | | | |
| 5 | 5_38789263_ | 38789263 | 38789161 | rs34836414 | G | A | 0.2944 | −0.0068 | 0.9932 | 0.9960 |
| | A_G | | | | | | | | | |
| 5 | 5_38820823_ | 38820823 | 38820721 | rs774802178 | CAT | C | 0.2979 | −0.0059 | 0.9941 | 0.9965 |
| | C_CAT | | | | | | | | | |
| 5 | 5_39582018_ | 39582018 | 39581916 | rs114212093 | C | A | 0.0127 | 0.0130 | 1.0131 | 1.0003 |
| | A_C | | | | | | | | | |
| 5 | 5_41638740_ | 41638740 | 41638638 | rs62360524 | C | G | 0.0413 | −0.0083 | 0.9917 | 0.9993 |
| | G_C | | | | | | | | | |
| 5 | 5_42529115_ | 42529115 | 42529013 | 5:42529115_C | C | CT | 0.3780 | −0.0014 | 0.9986 | 0.9989 |
| | CT_C | | | T_C | | | | | | |
| 5 | 5_43112570_ | 43112570 | 43112468 | rs13158202 | T | A | 0.1901 | −0.0007 | 0.9993 | 0.9997 |
| | A_T | | | | | | | | | |
| 5 | 5_43173849_ | 43173849 | 43173747 | | TC | T | 0.2318 | 0.0091 | 1.0091 | 1.0042 |
| | T_TC | | | | | | | | | |
| 5 | 5_43692598_ | 43692598 | 43692496 | rs71629192 | C | G | 0.0492 | 0.0000 | 1.0000 | 1.0000 |
| | G_C | | | | | | | | | |
| 5 | 5_44115401_ | 44115401 | 44115299 | rs36058457 | G | T | 0.1273 | −0.0116 | 0.9885 | 0.9971 |
| | T_G | | | | | | | | | |
| 5 | 5_44119344_ | 44119344 | 44119242 | rs62368138 | A | G | 0.0399 | 0.0248 | 1.0251 | 1.0020 |
| | G_A | | | | | | | | | |
| 5 | 5_44302177_ | 44302177 | 44302075 | rs13186320 | T | A | 0.5153 | 0.0064 | 1.0064 | 1.0066 |
| | A_T | | | | | | | | | |
| 5 | 5_44393569_ | 44393569 | 44393467 | rs10512852 | T | C | 0.0975 | 0.0000 | 1.0000 | 1.0000 |
| | C_T | | | | | | | | | |
| 5 | 5_44482570_ | 44482570 | 44482468 | rs35085334 | AT | A | 0.7325 | −0.0015 | 0.9985 | 0.9978 |
| | A_AT | | | | | | | | | |
| 5 | 5_44486733_ | 44486733 | 44486631 | rs72748067 | C | T | 0.0388 | −0.0013 | 0.9987 | 0.9999 |
| | T_C | | | | | | | | | |
| 5 | 5_44499367_ | 44499367 | 44499265 | 5:44499367_T | T | TA | 0.7735 | −0.0044 | 0.9956 | 0.9932 |
| | TA_T | | | A_T | | | | | | |
| 5 | 5_44508264_ | 44508264 | 44508162 | rs138335056 | GT | G | 0.1265 | −0.0743 | 0.9284 | 0.9820 |
| | G_GT | | | | | | | | | |
| 5 | 5_44609392_ | 44609392 | 44609290 | rs73093976 | T | C | 0.1692 | 0.0000 | 1.0000 | 1.0000 |
| | C_T | | | | | | | | | |
| 5 | 5_44619502_ | 44619502 | 44619400 | rs187108781 | G | A | 0.1549 | −0.0154 | 0.9847 | 0.9953 |
| | A_G | | | | | | | | | |
| 5 | 5_44621050_ | 44621050 | 44620948 | rs4866915 | C | T | 0.3804 | 0.0000 | 1.0000 | 1.0000 |
| | T_C | | | | | | | | | |
| 5 | 5_44645998_ | 44645998 | 44645896 | rs36068815 | C | T | 0.3997 | 0.0133 | 1.0134 | 1.0107 |
| | T_C | | | | | | | | | |
| 5 | 5_44649944_ | 44649944 | 44649842 | rs4613718 | T | C | 0.6010 | 0.0000 | 1.0000 | 1.0000 |
| | C_T | | | | | | | | | |
| 5 | 5_44691395_ | 44691395 | 44691293 | rs6451770 | G | T | 0.3837 | 0.0391 | 1.0399 | 1.0308 |
| | T_G | | | | | | | | | |
| 5 | 5_44706498_ | 44706498 | 44706396 | rs10941679 | G | A | 0.2481 | 0.0282 | 1.0286 | 1.0142 |
| | A_G | | | | | | | | | |
| 5 | 5_44800917_ | 44800917 | 44800815 | rs10462082 | C | A | 0.5032 | 0.0091 | 1.0091 | 1.0092 |
| | A_C | | | | | | | | | |
| 5 | 5_44875005_ | 44875005 | 44874903 | rs7716600 | C | A | 0.7825 | 0.0000 | 1.0000 | 1.0000 |
| | A_C | | | | | | | | | |
| 5 | 5_44875449_ | 44875449 | 44875347 | rs62366821 | G | A | 0.4846 | −0.0078 | 0.9922 | 0.9925 |
| | A_G | | | | | | | | | |
| 5 | 5_44922557_ | 44922557 | 44922455 | rs112234443 | T | C | 0.0490 | 0.0000 | 1.0000 | 1.0000 |
| | C_T | | | | | | | | | |
| 5 | 5_44929272_ | 44929272 | 44929170 | rs573963007 | T | A | 0.0254 | 0.0000 | 1.0000 | 1.0000 |
| | A_T | | | | | | | | | |
| 5 | 5_44947702_ | 44947702 | 44947600 | rs7701656 | C | T | 0.1262 | 0.0024 | 1.0024 | 1.0006 |
| | T_C | | | | | | | | | |
| 5 | 5_44977315_ | 44977315 | 44977213 | 5:44977315_A | A | ATAG | 0.0722 | 0.0227 | 1.0230 | 1.0033 |
| | ATAGT_A | | | TAGT_A | | T | | | | |
| 5 | 5_44981122_ | 44981122 | 44981020 | rs144854948 | A | G | 0.0149 | 0.0032 | 1.0032 | 1.0001 |
| | G_A | | | | | | | | | |
| 5 | 5_45018723_ | 45018723 | 45018621 | rs57909303 | A | G | 0.1202 | 0.0030 | 1.0030 | 1.0007 |
| | G_A | | | | | | | | | |
| 5 | 5_50162499_ | 50162499 | 50866665 | rs27267 | C | A | 0.4958 | −0.0002 | 0.9998 | 0.9998 |
| | A_C | | | | | | | | | |
| 5 | 5_50162788_ | 50162788 | 50866954 | rs27905 | A | G | 0.7477 | −0.0040 | 0.9960 | 0.9940 |
| | G_A | | | | | | | | | |
| 5 | 5_50172251_ | 50172251 | 50876417 | rs26089 | T | C | 0.3643 | −0.0003 | 0.9997 | 0.9998 |
| | C_T | | | | | | | | | |
| 5 | 5_50207118_ | 50207118 | 50911284 | rs32482 | C | T | 0.3864 | −0.0011 | 0.9989 | 0.9992 |
| | T_C | | | | | | | | | |
| 5 | 5_50635268_ | 50635268 | 51339434 | rs78812881 | A | G | 0.0310 | −0.0085 | 0.9915 | 0.9995 |
| | G_A | | | | | | | | | |
| 5 | 5_51204139_ | 51204139 | 51908305 | rs10652826 | CAA | C | 0.8702 | −0.0054 | 0.9946 | 0.9906 |
| | C_CAA | | | | | | | | | |
| 5 | 5_52377181_ | 52377181 | 53081351 | rs3212617 | A | T | 0.0557 | 0.0079 | 1.0079 | 1.0009 |
| | T_A | | | | | | | | | |
| 5 | 5_52499308_ | 52499308 | 53203478 | rs116520517 | A | G | 0.0187 | 0.0237 | 1.0240 | 1.0009 |
| | G_A | | | | | | | | | |
| 5 | 5_52585565_ | 52585565 | 53289735 | rs2591892 | A | T | 0.0295 | 0.0062 | 1.0062 | 1.0004 |
| | T_A | | | | | | | | | |
| 5 | 5_52679539_ | 52679539 | 53383709 | | CA | C | 0.0998 | 0.0231 | 1.0234 | 1.0047 |
| | C_CA | | | | | | | | | |
| 5 | 5_52768800_ | 52768800 | 53472970 | rs756147761 | C | CAT | 0.2389 | −0.0091 | 0.9909 | 0.9957 |
| | CAT_C | | | | | | | | | |
| 5 | 5_53222781_ | 53222781 | 53926951 | rs2253601 | T | A | 0.8456 | −0.0133 | 0.9868 | 0.9778 |
| | A_T | | | | | | | | | |
| 5 | 5_55621515_ | 55621515 | 56325688 | rs158489 | A | G | 0.0979 | 0.0002 | 1.0002 | 1.0000 |
| | G_A | | | | | | | | | |
| 5 | 5_55638158_ | 55638158 | 56342331 | rs56379516 | C | T | 0.2490 | 0.0000 | 1.0000 | 1.0000 |
| | T_C | | | | | | | | | |
| 5 | 5_55662540_ | 55662540 | 56366713 | rs553874618 | CT | C | 0.3631 | −0.0141 | 0.9860 | 0.9899 |
| | C_CT | | | | | | | | | |
| 5 | 5_55669065_ | 55669065 | 56373238 | rs7717167 | G | A | 0.3041 | −0.0052 | 0.9948 | 0.9968 |
| | A_G | | | | | | | | | |
| 5 | 5_55818087_ | 55818087 | 56522260 | rs166280 | T | C | 0.7602 | 0.0117 | 1.0118 | 1.0180 |
| | C_T | | | | | | | | | |
| 5 | 5_55883187_ | 55883187 | 56587360 | rs13155854 | T | C | 0.5262 | −0.0089 | 0.9911 | 0.9907 |
| | C_T | | | | | | | | | |
| 5 | 5_55912533_ | 55912533 | 56616706 | rs984113 | C | G | 0.6186 | −0.0231 | 0.9772 | 0.9719 |
| | G_C | | | | | | | | | |
| 5 | 5_55930546_ | 55930546 | 56634719 | rs1279628 | T | G | 0.4470 | −0.0004 | 0.9996 | 0.9996 |
| | G_T | | | | | | | | | |
| 5 | 5_55965167_ | 55965167 | 56669340 | rs889310 | T | C | 0.5576 | 0.0068 | 1.0068 | 1.0076 |
| | C_T | | | | | | | | | |
| 5 | 5_55965608_ | 55965608 | 56669781 | rs16885969 | A | G | 0.2421 | −0.0014 | 0.9986 | 0.9993 |
| | G_A | | | | | | | | | |
| 5 | 5_55989045_ | 55989045 | 56693218 | rs61657998 | T | G | 0.1218 | 0.0000 | 1.0000 | 1.0000 |
| | G_T | | | | | | | | | |
| 5 | 5_55989304_ | 55989304 | 56693477 | rs1862624 | A | G | 0.5749 | −0.0055 | 0.9945 | 0.9937 |
| | G_A | | | | | | | | | |
| 5 | 5_56012629_ | 56012629 | 56716802 | rs548650585 | TA | T | 0.0580 | 0.0000 | 1.0000 | 1.0000 |
| | T_TA | | | | | | | | | |
| 5 | 5_56023083_ | 56023083 | 56727256 | rs16886165 | G | T | 0.1583 | 0.0909 | 1.0952 | 1.0304 |
| | T_G | | | | | | | | | |
| 5 | 5_56026500_ | 56026500 | 56730673 | rs1910020 | C | T | 0.7238 | −0.0055 | 0.9945 | 0.9921 |
| | T_C | | | | | | | | | |
| 5 | 5_56042972_ | 56042972 | 56747145 | rs76250845 | T | C | 0.0521 | 0.0190 | 1.0192 | 1.0020 |
| | C_T | | | | | | | | | |
| 5 | 5_56045081_ | 56045081 | 56749254 | rs11949391 | C | T | 0.1655 | −0.0186 | 0.9816 | 0.9939 |
| | T_C | | | | | | | | | |
| 5 | 5_56060954_ | 56060954 | 56765127 | rs112497245 | A | G | 0.0621 | 0.0000 | 1.0000 | 1.0000 |
| | G_A | | | | | | | | | |
| 5 | 5_56068859_ | 56068859 | 56773032 | 5:56068859_A | A | AC | 0.1679 | 0.0322 | 1.0327 | 1.0110 |
| | AC_A | | | C_A | | | | | | |
| 5 | 5_56110374_ | 56110374 | 56814547 | rs760596453 | G | GGAC | 0.6510 | −0.0042 | 0.9958 | 0.9946 |
| | GGACCTGGCC | | | | | CTGG | | | | |
| | GA_G | | | | | CCGA | | | | |
| 5 | 5_56110937_ | 56110937 | 56815110 | rs72758040 | C | G | 0.2508 | −0.0014 | 0.9986 | 0.9993 |
| | G_C | | | | | | | | | |
| 5 | 5_56133039_ | 56133039 | 56837212 | rs72758067 | G | A | 0.0204 | 0.0000 | 1.0000 | 1.0000 |
| | A_G | | | | | | | | | |
| 5 | 5_56153470_ | 56153470 | 56857643 | rs183088996 | C | T | 0.0157 | 0.0002 | 1.0002 | 1.0000 |
| | T_C | | | | | | | | | |
| 5 | 5_56254772_ | 56254772 | 56958945 | rs74571895 | G | A | 0.0540 | 0.0414 | 1.0423 | 1.0046 |
| | A_G | | | | | | | | | |
| 5 | 5_56260606_ | 56260606 | 56964779 | rs6890270 | G | A | 0.1864 | 0.0020 | 1.0020 | 1.0007 |
| | A_G | | | | | | | | | |
| 5 | 5_57186663_ | 57186663 | 57890836 | rs35100619 | A | G | 0.0899 | 0.0074 | 1.0074 | 1.0013 |
| | G_A | | | | | | | | | |
| 5 | 5_57595040_ | 57595040 | 58299213 | 5:57595040_C | C | CTTT | 0.0705 | 0.0171 | 1.0172 | 1.0024 |
| | CTTT_C | | | TTT_C | | | | | | |
| 5 | 5_57600833_ | 57600833 | 58305006 | rs1202501 | A | G | 0.2162 | 0.0014 | 1.0014 | 1.0006 |
| | G_A | | | | | | | | | |
| 5 | 5_57633272_ | 57633272 | 58337445 | rs245284 | C | T | 0.0665 | 0.0039 | 1.0039 | 1.0005 |
| | T_C | | | | | | | | | |
| 5 | 5_58177785_ | 58177785 | 58881958 | rs79517802 | C | G | 0.0436 | −0.0070 | 0.9930 | 0.9994 |
| | G_C | | | | | | | | | |
| 5 | 5_58184061_ | 58184061 | 58888234 | rs10472076 | C | T | 0.3776 | 0.0070 | 1.0070 | 1.0053 |
| | T_C | | | | | | | | | |
| 5 | 5_58229580_ | 58229580 | 58933753 | rs2944383 | G | C | 0.6057 | −0.0012 | 0.9988 | 0.9985 |
| | C_G | | | | | | | | | |
| 5 | 5_58241712_ | 58241712 | 58945885 | rs113778879 | T | C | 0.5750 | −0.0029 | 0.9971 | 0.9967 |
| | C_T | | | | | | | | | |
| 5 | 5_58282789_ | 58282789 | 58986962 | | GATAT | G | 0.6482 | 0.0000 | 1.0000 | 1.0000 |
| | G_GATAT | | | | | | | | | |
| 5 | 5_58306572_ | 58306572 | 59010745 | rs111567606 | C | A | 0.0927 | −0.0091 | 0.9909 | 0.9983 |
| | A_C | | | | | | | | | |
| 5 | 5_58365353_ | 58365353 | 59069526 | rs537267133 | C | T | 0.0917 | −0.0138 | 0.9863 | 0.9975 |
| | T_C | | | | | | | | | |
| 5 | 5_58427472_ | 58427472 | 59131645 | rs28373501 | T | C | 0.7101 | 0.0022 | 1.0022 | 1.0031 |
| | C_T | | | | | | | | | |
| 5 | 5_58442025_ | 58442025 | 59146198 | rs12658004 | T | A | 0.5177 | −0.0026 | 0.9974 | 0.9973 |
| | A_T | | | | | | | | | |
| 5 | 5_58476117_ | 58476117 | 59180291 | rs9292195 | C | T | 0.8508 | −0.0064 | 0.9936 | 0.9892 |
| | T_C | | | | | | | | | |
| 5 | 5_58498540_ | 58498540 | 59202714 | rs1439 | A | G | 0.1701 | −0.0002 | 0.9998 | 0.9999 |
| | G_A | | | | | | | | | |
| 5 | 5_58720965_ | 58720965 | 59425139 | rs2702371 | C | T | 0.4967 | −0.0037 | 0.9963 | 0.9963 |
| | T_C | | | | | | | | | |
| 5 | 5_58826435_ | 58826435 | 59530609 | 5:58826435_T | T | TA | 0.4063 | −0.0003 | 0.9997 | 0.9998 |
| | TA_T | | | A_T | | | | | | |
| 5 | 5_59717871_ | 59717871 | 60422044 | rs186842595 | C | T | 0.0165 | 0.0056 | 1.0056 | 1.0002 |
| | T_C | | | | | | | | | |
| 5 | 5_60956369_ | 60956369 | 61660542 | rs79488625 | C | G | 0.1830 | 0.0095 | 1.0095 | 1.0035 |
| | G_C | | | | | | | | | |
| 5 | 5_60971732_ | 60971732 | 61675905 | rs432733 | A | G | 0.1555 | 0.0005 | 1.0005 | 1.0002 |
| | G_A | | | | | | | | | |
| 5 | 5_63913119_ | 63913119 | 64617292 | rs2216706 | T | C | 0.8624 | −0.0083 | 0.9917 | 0.9858 |
| | C_T | | | | | | | | | |
| 5 | 5_64319641_ | 64319641 | 65023814 | rs150001891 | AAG | A | 0.4476 | −0.0012 | 0.9988 | 0.9989 |
| | A_AAG | | | | | | | | | |
| 5 | 5_64373628_ | 64373628 | 65077801 | rs61482254 | A | C | 0.3235 | −0.0056 | 0.9944 | 0.9964 |
| | C_A | | | | | | | | | |
| 5 | 5_64526683_ | 64526683 | 65230856 | rs754861492 | AATAT | A | 0.3598 | −0.0049 | 0.9951 | 0.9965 |
| | A_AATATATAT | | | | ATATAT | | | | | |
| | AT | | | | | | | | | |
| 5 | 5_64624361_ | 64624361 | 65328534 | 5:64624361_T | T | TA | 0.5104 | −0.0037 | 0.9963 | 0.9962 |
| | TA_T | | | A_T | | | | | | |
| 5 | 5_64640912_ | 64640912 | 65345085 | rs2640717 | A | G | 0.3778 | 0.0038 | 1.0038 | 1.0029 |
| | G_A | | | | | | | | | |
| 5 | 5_64705169_ | 64705169 | 65409342 | rs534131894 | CAA | C | 0.3855 | 0.0000 | 1.0000 | 1.0000 |
| | C_CAA | | | | | | | | | |
| 5 | 5_66979250_ | 66979250 | 67683422 | rs6449898 | T | G | 0.2984 | 0.0002 | 1.0002 | 1.0001 |
| | G_T | | | | | | | | | |
| 5 | 5_66983803_ | 66983803 | 67687975 | rs60493723 | T | C | 0.2120 | 0.0048 | 1.0048 | 1.0020 |
| | C_T | | | | | | | | | |
| 5 | 5_67539174_ | 67539174 | 68243346 | rs56351136 | G | T | 0.1307 | −0.0110 | 0.9891 | 0.9971 |
| | T_G | | | | | | | | | |
| 5 | 5_67597321_ | 67597321 | 68301493 | rs9623 | C | T | 0.4440 | −0.0008 | 0.9992 | 0.9993 |
| | T_C | | | | | | | | | |
| 5 | 5_67611486_ | 67611486 | 68315658 | rs184886 | C | T | 0.7259 | 0.0024 | 1.0024 | 1.0035 |
| | T_C | | | | | | | | | |
| 5 | 5_67774099_ | 67774099 | 68478272 | rs78909682 | G | C | 0.1032 | 0.0009 | 1.0009 | 1.0002 |
| | C_G | | | | | | | | | |
| 5 | 5_68537510_ | 68537510 | 69241683 | rs114456509 | G | T | 0.0145 | −0.0622 | 0.9397 | 0.9982 |
| | T_G | | | | | | | | | |
| 5 | 5_71509063_ | 71509063 | 72213236 | rs62363244 | G | A | 0.3362 | −0.0118 | 0.9883 | 0.9921 |
| | A_G | | | | | | | | | |
| 5 | 5_71664031_ | 71664031 | 72368204 | rs17377355 | A | G | 0.3391 | −0.0010 | 0.9990 | 0.9993 |
| | G_A | | | | | | | | | |
| 5 | 5_71732116_ | 71732116 | 72436289 | 5:71732116_T | T | TTA | 0.7400 | −0.0011 | 0.9989 | 0.9984 |
| | TTA_T | | | TA_T | | | | | | |
| 5 | 5_71965007_ | 71965007 | 72669180 | rs3010266 | A | G | 0.2572 | −0.0133 | 0.9868 | 0.9932 |
| | G_A | | | | | | | | | |
| 5 | 5_72176643_ | 72176643 | 72880816 | rs35726893 | CAA | C | 0.4488 | 0.0008 | 1.0008 | 1.0007 |
| | C_CAA | | | | | | | | | |
| 5 | 5_72412780_ | 72412780 | 73116953 | rs602307 | A | T | 0.4111 | 0.0024 | 1.0024 | 1.0020 |
| | T_A | | | | | | | | | |
| 5 | 5_73226271_ | 73226271 | 73930446 | rs283629 | C | T | 0.3545 | −0.0124 | 0.9877 | 0.9913 |
| | T_C | | | | | | | | | |
| 5 | 5_73234583_ | 73234583 | 73938758 | rs157557 | C | T | 0.3213 | −0.0102 | 0.9899 | 0.9935 |
| | T_C | | | | | | | | | |
| 5 | 5_73274360_ | 73274360 | 73978535 | rs425101 | C | G | 0.3265 | 0.0000 | 1.0000 | 1.0000 |
| | G_C | | | | | | | | | |
| 5 | 5_73397165_ | 73397165 | 74101340 | 5:73397165_A | A | AATT | 0.0803 | −0.0103 | 0.9898 | 0.9984 |
| | AATTG_A | | | ATTG_A | | G | | | | |
| 5 | 5_77154447_ | 77154447 | 77858623 | rs6887123 | C | G | 0.5006 | −0.0081 | 0.9919 | 0.9919 |
| | G_C | | | | | | | | | |
| 5 | 5_77155397_ | 77155397 | 77859573 | rs767431357 | G | GT | 0.3466 | −0.0145 | 0.9856 | 0.9900 |
| | GT_G | | | | | | | | | |
| 5 | 5_79180995_ | 79180995 | 79885172 | rs34525310 | GA | G | 0.1755 | 0.0065 | 1.0065 | 1.0023 |
| | G_GA | | | | | | | | | |
| 5 | 5_79228649_ | 79228649 | 79932826 | rs6453492 | G | A | 0.6101 | 0.0000 | 1.0000 | 1.0000 |
| | A_G | | | | | | | | | |
| 5 | 5_81091203_ | 81091203 | 81795384 | rs6885263 | C | A | 0.1419 | −0.0101 | 0.9900 | 0.9972 |
| | A_C | | | | | | | | | |
| 5 | 5_81146156_ | 81146156 | 81850337 | rs17241969 | C | T | 0.0966 | 0.0000 | 1.0000 | 1.0000 |
| | T_C | | | | | | | | | |
| 5 | 5_81214843_ | 81214843 | 81919024 | rs17302470 | A | T | 0.1449 | 0.0000 | 1.0000 | 1.0000 |
| | T_A | | | | | | | | | |
| 5 | 5_81512947_ | 81512947 | 82217128 | 5:81512947_T | T | TA | 0.2503 | −0.0379 | 0.9628 | 0.9815 |
| | TA_T | | | A_T | | | | | | |
| 5 | 5_83156500_ | 83156500 | 83860681 | rs10044480 | T | C | 0.2915 | 0.0099 | 1.0099 | 1.0058 |
| | C_T | | | | | | | | | |
| 5 | 5_83159933_ | 83159933 | 83864114 | rs56000039 | G | A | 0.3099 | 0.0055 | 1.0055 | 1.0034 |
| | A_G | | | | | | | | | |
| 5 | 5_83165631_ | 83165631 | 83869812 | rs6452552 | A | C | 0.7437 | 0.0106 | 1.0107 | 1.0159 |
| | C_A | | | | | | | | | |
| 5 | 5_83315048_ | 83315048 | 84019229 | rs253158 | G | A | 0.9171 | −0.0016 | 0.9984 | 0.9971 |
| | A_G | | | | | | | | | |
| 5 | 5_83321882_ | 83321882 | 84026063 | rs442848 | T | C | 0.9266 | −0.0028 | 0.9972 | 0.9948 |
| | C_T | | | | | | | | | |
| 5 | 5_87544144_ | 87544144 | 88248327 | rs59198891 | T | C | 0.0119 | 0.0294 | 1.0298 | 1.0007 |
| | C_T | | | | | | | | | |
| 5 | 5_90647770_ | 90647770 | 91351953 | rs4916712 | C | T | 0.4600 | −0.0003 | 0.9997 | 0.9997 |
| | T_C | | | | | | | | | |
| 5 | 5_90747018_ | 90747018 | 91451201 | 5:90747018_G | G | GA | 0.1770 | −0.0213 | 0.9789 | 0.9926 |
| | GA_G | | | A_G | | | | | | |
| 5 | 5_90788643_ | 90788643 | 91492826 | 5:90788643_A | A | ATAT | 0.0946 | 0.0000 | 1.0000 | 1.0000 |
| | ATATTTC_A | | | TATTTC_A | | TTC | | | | |
| 5 | 5_90789470_ | 90789470 | 91493653 | rs332529 | A | G | 0.1580 | −0.0086 | 0.9914 | 0.9973 |
| | G_A | | | | | | | | | |
| 5 | 5_90926353_ | 90926353 | 91630536 | rs4492142 | G | T | 0.5074 | −0.0058 | 0.9942 | 0.9941 |
| | T_G | | | | | | | | | |
| 5 | 5_91050109_ | 91050109 | 91754292 | rs13166131 | G | T | 0.5488 | 0.0043 | 1.0043 | 1.0047 |
| | T_G | | | | | | | | | |
| 5 | 5_92743235_ | 92743235 | 93407529 | rs73133209 | T | C | 0.0232 | −0.0138 | 0.9863 | 0.9994 |
| | C_T | | | | | | | | | |
| 5 | 5_94268466_ | 94268466 | 94932761 | rs32909 | C | T | 0.3196 | −0.0041 | 0.9959 | 0.9974 |
| | T_C | | | | | | | | | |
| 5 | 5_95147211_ | 95147211 | 95811507 | rs11953221 | T | C | 0.0288 | 0.0241 | 1.0244 | 1.0014 |
| | C_T | | | | | | | | | |
| 5 | 5_96169897_ | 96169897 | 96834194 | rs10476692 | C | T | 0.0659 | −0.0066 | 0.9934 | 0.9991 |
| | T_C | | | | | | | | | |
| 5 | 5_97823498_ | 97823498 | 98487794 | rs10463479 | C | T | 0.2807 | 0.0040 | 1.0040 | 1.0023 |
| | T_C | | | | | | | | | |
| 5 | 5_103478992_ | 103478992 | 104143291 | rs140135691 | T | G | 0.0146 | −0.0100 | 0.9900 | 0.9997 |
| | G_T | | | | | | | | | |
| 5 | 5_104223498_ | 104223498 | 104887797 | rs1217465 | T | A | 0.5291 | 0.0023 | 1.0023 | 1.0024 |
| | A_T | | | | | | | | | |
| 5 | 5_104300273_ | 104300273 | 104964572 | rs17157372 | T | G | 0.1810 | −0.0098 | 0.9902 | 0.9965 |
| | G_T | | | | | | | | | |
| 5 | 5_104302751_ | 104302751 | 104967050 | rs6596590 | T | A | 0.1605 | −0.0078 | 0.9922 | 0.9975 |
| | A_T | | | | | | | | | |
| 5 | 5_104331054_ | 104331054 | 104995353 | rs7445672 | G | T | 0.8310 | 0.0004 | 1.0004 | 1.0007 |
| | T_G | | | | | | | | | |
| 5 | 5_108542241_ | 108542241 | 109206540 | rs200459449 | CA | C | 0.1377 | 0.0000 | 1.0000 | 1.0000 |
| | C_CA | | | | | | | | | |
| 5 | 5_108572649_ | 108572649 | 109236948 | rs75073283 | G | A | 0.0827 | 0.0000 | 1.0000 | 1.0000 |
| | A_G | | | | | | | | | |
| 5 | 5_110657114_ | 110657114 | 111321416 | rs1660930 | G | A | 0.5854 | −0.0046 | 0.9954 | 0.9946 |
| | A_G | | | | | | | | | |
| 5 | 5_111103258_ | 111103258 | 111767561 | rs255888 | T | C | 0.5586 | 0.0063 | 1.0063 | 1.0071 |
| | C_T | | | | | | | | | |
| 5 | 5_111217786_ | 111217786 | 111882089 | rs6882649 | T | G | 0.6646 | 0.0059 | 1.0059 | 1.0079 |
| | G_T | | | | | | | | | |
| 5 | 5_111368597_ | 111368597 | 112032900 | 5:111368597_ | T | TAAA | 0.0499 | −0.0110 | 0.9891 | 0.9989 |
| | TAAATA_T | | | TAAATA_T | | TA | | | | |
| 5 | 5_114066225_ | 114066225 | 114730528 | rs66616485 | G | T | 0.0688 | 0.0196 | 1.0198 | 1.0027 |
| | T_G | | | | | | | | | |
| 5 | 5_114525350_ | 114525350 | 115189653 | rs17381680 | T | G | 0.1199 | 0.0182 | 1.0184 | 1.0044 |
| | G_T | | | | | | | | | |
| 5 | 5_116745252_ | 116745252 | 117409556 | 5:116745252_ | T | TGTG | 0.6591 | −0.0013 | 0.9987 | 0.9983 |
| | TGTG_T | | | TGTG_T | | | | | | |
| 5 | 5_118704975_ | 118704975 | 119369280 | rs148486181 | T | A | 0.0436 | 0.0220 | 1.0222 | 1.0019 |
| | A_T | | | | | | | | | |
| 5 | 5_120888598_ | 120888598 | 121552903 | rs9327200 | A | T | 0.8574 | 0.0000 | 1.0000 | 1.0000 |
| | T_A | | | | | | | | | |
| 5 | 5_121789141_ | 121789141 | 122453446 | rs2737093 | A | G | 0.1364 | 0.0068 | 1.0068 | 1.0019 |
| | G_A | | | | | | | | | |
| 5 | 5_122436528_ | 122436528 | 123100833 | rs163188 | G | C | 0.0902 | 0.0031 | 1.0031 | 1.0006 |
| | C_G | | | | | | | | | |
| 5 | 5_122442322_ | 122442322 | 123106627 | rs335143 | C | A | 0.1824 | 0.0041 | 1.0041 | 1.0015 |
| | A_C | | | | | | | | | |
| 5 | 5_122478676_ | 122478676 | 123142982 | rs335160 | A | C | 0.7448 | −0.0066 | 0.9934 | 0.9902 |
| | C_A | | | | | | | | | |
| 5 | 5_122705244_ | 122705244 | 123369550 | rs1428387 | T | C | 0.0306 | 0.0286 | 1.0290 | 1.0018 |
| | C_T | | | | | | | | | |
| 5 | 5_122734724_ | 122734724 | 123399030 | rs547152106 | TA | T | 0.3982 | −0.0020 | 0.9980 | 0.9984 |
| | T_TA | | | | | | | | | |
| 5 | 5_124012247_ | 124012247 | 124676554 | rs114652469 | A | G | 0.0207 | −0.0076 | 0.9924 | 0.9997 |
| | G_A | | | | | | | | | |
| 5 | 5_124110347_ | 124110347 | 124774654 | rs780903759 | A | AACA | 0.3674 | 0.0005 | 1.0005 | 1.0004 |
| | AACAT_A | | | | | T | | | | |
| 5 | 5_125327257_ | 125327257 | 125991564 | rs78389812 | C | T | 0.0235 | −0.0182 | 0.9820 | 0.9992 |
| | T_C | | | | | | | | | |
| 5 | 5_125431223_ | 125431223 | 126095530 | | T | TA | 0.9322 | −0.0128 | 0.9873 | 0.9764 |
| | TA_T | | | | | | | | | |
| 5 | 5_127418850_ | 127418850 | 128083158 | rs3812049 | G | C | 0.7598 | −0.0032 | 0.9968 | 0.9952 |
| | C_G | | | | | | | | | |
| 5 | 5_127596305_ | 127596305 | 128260613 | rs144549501 | A | G | 0.0251 | −0.0166 | 0.9835 | 0.9992 |
| | G_A | | | | | | | | | |
| 5 | 5_128290369_ | 128290369 | 128954676 | | GTA | G | 0.0763 | −0.0094 | 0.9906 | 0.9986 |
| | G_GTA | | | | | | | | | |
| 5 | 5_128648201_ | 128648201 | 129312508 | rs151158087 | G | A | 0.5281 | −0.0076 | 0.9924 | 0.9920 |
| | A_G | | | | | | | | | |
| 5 | 5_128652984_ | 128652984 | 129317291 | rs113428195 | T | C | 0.0380 | 0.0178 | 1.0180 | 1.0014 |
| | C_T | | | | | | | | | |
| 5 | 5_128664078_ | 128664078 | 129328385 | rs149224778 | ATTGTT | A | 0.2706 | −0.0044 | 0.9956 | 0.9976 |
| | A_ATTGTT | | | | | | | | | |
| 5 | 5_131577849_ | 131577849 | 132242156 | rs112951115 | CCTGCTT | C | 0.5289 | 0.0015 | 1.0015 | 1.0016 |
| | C_CCTGCTTTCT | | | | TCT | | | | | |
| 5 | 5_131640536_ | 131640536 | 132304843 | rs6860806 | G | A | 0.5427 | 0.0084 | 1.0084 | 1.0092 |
| | A_G | | | | | | | | | |
| 5 | 5_131667353_ | 131667353 | 132331660 | rs273909 | G | A | 0.1167 | −0.0029 | 0.9971 | 0.9993 |
| | A_G | | | | | | | | | |
| 5 | 5_131808869_ | 131808869 | 132473177 | rs2548993 | A | G | 0.7345 | 0.0004 | 1.0004 | 1.0006 |
| | G_A | | | | | | | | | |
| 5 | 5_131833599_ | 131833599 | 132497907 | rs736801 | T | C | 0.3629 | 0.0101 | 1.0102 | 1.0074 |
| | C_T | | | | | | | | | |
| 5 | 5_132407058_ | 132407058 | 133071366 | rs6596100 | T | C | 0.2450 | −0.0183 | 0.9819 | 0.9911 |
| | C_T | | | | | | | | | |
| 5 | 5_135572910_ | 135572910 | 136237222 | rs889010 | A | C | 0.5683 | −0.0033 | 0.9967 | 0.9963 |
| | C_A | | | | | | | | | |
| 5 | 5_135619617_ | 135619617 | 136283929 | rs1499769 | T | G | 0.1233 | −0.0026 | 0.9974 | 0.9994 |
| | G_T | | | | | | | | | |
| 5 | 5_135686384_ | 135686384 | 136350696 | | G | C | 0.5218 | 0.0095 | 1.0095 | 1.0100 |
| | C_G | | | | | | | | | |
| 5 | 5_135882597_ | 135882597 | 136546908 | rs4340911 | G | C | 0.7011 | 0.0002 | 1.0002 | 1.0003 |
| | C_G | | | | | | | | | |
| 5 | 5_141462248_ | 141462248 | 142082683 | 5:141462248_ | T | TA | 0.0152 | −0.0263 | 0.9740 | 0.9992 |
| | TA_T | | | TA_T | | | | | | |
| 5 | 5_141669839_ | 141669839 | 142290274 | rs10051199 | T | G | 0.4822 | −0.0096 | 0.9904 | 0.9908 |
| | G_T | | | | | | | | | |
| 5 | 5_141760963_ | 141760963 | 142381398 | | ATTTTTT | A | 0.5510 | −0.0001 | 0.9999 | 0.9999 |
| | A_ATTTTTTTGT | | | | TGTTTTG | | | | | |
| | TTTGTTTTGTTT | | | | TTTTGT | | | | | |
| | TG | | | | TTTG | | | | | |
| 5 | 5_141764170_ | 141764170 | 142384605 | rs254442 | A | T | 0.5106 | −0.0098 | 0.9902 | 0.9901 |
| | T_A | | | | | | | | | |
| 5 | 5_142756004_ | 142756004 | 143376439 | rs2963155 | G | A | 0.2444 | −0.0024 | 0.9976 | 0.9988 |
| | A_G | | | | | | | | | |
| 5 | 5_142979991_ | 142979991 | 143600426 | rs153515 | C | A | 0.4667 | −0.0098 | 0.9902 | 0.9909 |
| | A_C | | | | | | | | | |
| 5 | 5_143357278_ | 143357278 | 143977713 | rs318403 | T | C | 0.5555 | 0.0087 | 1.0087 | 1.0097 |
| | C_T | | | | | | | | | |
| 5 | 5_143837325_ | 143837325 | 144457762 | rs9324959 | A | G | 0.4009 | −0.0058 | 0.9942 | 0.9954 |
| | G_A | | | | | | | | | |
| 5 | 5_144071035_ | 144071035 | 144691472 | 5:144071035_ | C | CT | 0.3330 | −0.0063 | 0.9937 | 0.9958 |
| | CT_C | | | CT_C | | | | | | |
| 5 | 5_144691771_ | 144691771 | 145312208 | rs1597465 | C | G | 0.3695 | −0.0019 | 0.9981 | 0.9986 |
| | G_C | | | | | | | | | |
| 5 | 5_144694693_ | 144694693 | 145315130 | rs112907216 | T | C | 0.0491 | −0.0071 | 0.9929 | 0.9993 |
| | C_T | | | | | | | | | |
| 5 | 5_146084418_ | 146084418 | 146704855 | rs12659673 | A | T | 0.0940 | −0.0137 | 0.9864 | 0.9974 |
| | T_A | | | | | | | | | |
| 5 | 5_146330004_ | 146330004 | 146950441 | rs1036202 | A | T | 0.1893 | −0.0013 | 0.9987 | 0.9995 |
| | T_A | | | | | | | | | |
| 5 | 5_146434047_ | 146434047 | 147054484 | 5:146434047_ | T | TA | 0.7610 | 0.0151 | 1.0152 | 1.0233 |
| | TA_T | | | TA_T | | | | | | |
| 5 | 5_148429004_ | 148429004 | 149049441 | rs36081 | G | A | 0.4326 | 0.0044 | 1.0044 | 1.0038 |
| | A_G | | | | | | | | | |
| 5 | 5_148510232_ | 148510232 | 149130669 | rs6889911 | T | C | 0.9733 | −0.0120 | 0.9881 | 0.9769 |
| | C_T | | | | | | | | | |
| 5 | 5_148520923_ | 148520923 | 149141360 | rs3839235 | ACACAAG | A | 0.7132 | −0.0064 | 0.9936 | 0.9909 |
| | A_ACACAAGGCC | | | | GCCTCGT | | | | | |
| | TCGTCCATCT | | | | CCATCT | | | | | |
| 5 | 5_148621845_ | 148621845 | 149242282 | rs41291953 | A | G | 0.0186 | −0.0330 | 0.9675 | 0.9988 |
| | G_A | | | | | | | | | |
| 5 | 5_154041399_ | 154041399 | 154661839 | rs13177281 | A | G | 0.1186 | −0.0081 | 0.9919 | 0.9981 |
| | G_A | | | | | | | | | |
| 5 | 5_154355695_ | 154355695 | 154976135 | rs6890610 | T | C | 0.2577 | −0.0037 | 0.9963 | 0.9981 |
| | C_T | | | | | | | | | |
| 5 | 5_155470446_ | 155470446 | 156043436 | rs4704943 | C | T | 0.2304 | −0.0026 | 0.9974 | 0.9988 |
| | T_C | | | | | | | | | |
| 5 | 5_155497189_ | 155497189 | 156070179 | rs139257230 | T | C | 0.1998 | −0.0095 | 0.9905 | 0.9962 |
| | C_T | | | | | | | | | |
| 5 | 5_156489247_ | 156489247 | 157062236 | rs11952686 | A | G | 0.0167 | −0.0235 | 0.9768 | 0.9992 |
| | G_A | | | | | | | | | |
| 5 | 5_157616208_ | 157616208 | 158189200 | rs17055363 | G | A | 0.1080 | −0.0210 | 0.9792 | 0.9955 |
| | A_G | | | | | | | | | |
| 5 | 5_157641763_ | 157641763 | 158214755 | rs11741271 | A | C | 0.1521 | 0.0019 | 1.0019 | 1.0006 |
| | C_A | | | | | | | | | |
| 5 | 5_158036093_ | 158036093 | 158609085 | rs10462916 | A | G | 0.3136 | 0.0075 | 1.0075 | 1.0047 |
| | G_A | | | | | | | | | |
| 5 | 5_158122566_ | 158122566 | 158695558 | rs79011982 | A | G | 0.0706 | 0.0000 | 1.0000 | 1.0000 |
| | G_A | | | | | | | | | |
| 5 | 5_158136337_ | 158136337 | 158709329 | rs113926471 | A | T | 0.0254 | 0.0020 | 1.0020 | 1.0001 |
| | T_A | | | | | | | | | |
| 5 | 5_158202753_ | 158202753 | 158775745 | | AACACAC | AACA | 0.2493 | 0.0089 | 1.0089 | 1.0045 |
| | AACACAC_ | | | | AC | CAC | | | | |
| | AACACACAC | | | | | | | | | |
| 5 | 5_158244083_ | 158244083 | 158817075 | rs1432679 | T | C | 0.5683 | −0.0200 | 0.9802 | 0.9776 |
| | C_T | | | | | | | | | |
| 5 | 5_158261163_ | 158261163 | 158834155 | rs17715065 | T | C | 0.5126 | −0.0150 | 0.9851 | 0.9848 |
| | C_T | | | | | | | | | |
| 5 | 5_158263144_ | 158263144 | 158836136 | rs6885822 | A | G | 0.1491 | 0.0000 | 1.0000 | 1.0000 |
| | G_A | | | | | | | | | |
| 5 | 5_158270225_ | 158270225 | 158843217 | rs7707918 | C | A | 0.3943 | −0.0060 | 0.9940 | 0.9953 |
| | A_C | | | | | | | | | |
| 5 | 5_158566745_ | 158566745 | 159139737 | rs2420354 | G | A | 0.4272 | −0.0022 | 0.9978 | 0.9981 |
| | A_G | | | | | | | | | |
| 5 | 5_162232358_ | 162232358 | 162805352 | rs1288205 | A | G | 0.3070 | −0.0048 | 0.9952 | 0.9971 |
| | G_A | | | | | | | | | |
| 5 | 5_162974403_ | 162974403 | 163547397 | rs7733433 | C | T | 0.1876 | 0.0068 | 1.0068 | 1.0026 |
| | T_C | | | | | | | | | |
| 5 | 5_165664201_ | 165664201 | 166237196 | rs62382837 | C | T | 0.0696 | −0.0090 | 0.9910 | 0.9988 |
| | T_C | | | | | | | | | |
| 5 | 5_166176342_ | 166176342 | 166749337 | rs114356534 | C | A | 0.0219 | 0.0135 | 1.0136 | 1.0006 |
| | A_C | | | | | | | | | |
| 5 | 5_166259411_ | 166259411 | 166832406 | rs113755300 | T | G | 0.0527 | −0.0232 | 0.9771 | 0.9976 |
| | G_T | | | | | | | | | |
| 5 | 5_166337984_ | 166337984 | 166910979 | rs13180091 | C | T | 0.1948 | −0.0080 | 0.9920 | 0.9969 |
| | T_C | | | | | | | | | |
| 5 | 5_166361214_ | 166361214 | 166934209 | rs34067733 | A | G | 0.1531 | 0.0120 | 1.0121 | 1.0037 |
| | G_A | | | | | | | | | |
| 5 | 5_169511279_ | 169511279 | 170084275 | rs6872596 | A | C | 0.3805 | 0.0000 | 1.0000 | 1.0000 |
| | C_A | | | | | | | | | |
| 5 | 5_169512748_ | 169512748 | 170085744 | rs17562083 | A | C | 0.3536 | 0.0048 | 1.0048 | 1.0034 |
| | C_A | | | | | | | | | |
| 5 | 5_169534173_ | 169534173 | 170107169 | rs4315934 | G | A | 0.5784 | −0.0022 | 0.9978 | 0.9975 |
| | A_G | | | | | | | | | |
| 5 | 5_169543263_ | 169543263 | 170116259 | | AGATGGA | AGAT | 0.1940 | 0.0000 | 1.0000 | 1.0000 |
| | AGATG_AGATGG | | | | TG | G | | | | |
| | ATG | | | | | | | | | |
| 5 | 5_169549409_ | 169549409 | 170122405 | rs141587449 | AAAGAAG | A | 0.7845 | 0.0070 | 1.0070 | 1.0111 |
| | A_AAAGAAG | | | | | | | | | |
| 5 | 5_169591460_ | 169591460 | 170164456 | rs10074269 | C | T | 0.3345 | 0.0147 | 1.0148 | 1.0099 |
| | T_C | | | | | | | | | |
| 5 | 5_169610853_ | 169610853 | 170183849 | rs80189533 | A | G | 0.2548 | −0.0058 | 0.9942 | 0.9971 |
| | G_A | | | | | | | | | |
| 5 | 5_173358154_ | 173358154 | 173931151 | rs6864691 | A | G | 0.4074 | 0.0116 | 1.0117 | 1.0095 |
| | G_A | | | | | | | | | |
| 5 | 5_173650528_ | 173650528 | 174223525 | rs10476105 | A | C | 0.1082 | −0.0037 | 0.9963 | 0.9992 |
| | C_A | | | | | | | | | |
| 5 | 5_173864492_ | 173864492 | 174437489 | rs35488892 | A | C | 0.2263 | −0.0031 | 0.9969 | 0.9986 |
| | C_A | | | | | | | | | |
| 5 | 5_173964594_ | 173964594 | 174537591 | rs765642088 | GTT | GT | 0.0996 | −0.0106 | 0.9895 | 0.9979 |
| | GT_GTT | | | | | | | | | |
| 5 | 5_176134882_ | 176134882 | 176707881 | rs4868701 | C | T | 0.5422 | 0.0120 | 1.0121 | 1.0131 |
| | T_C | | | | | | | | | |
| 5 | 5_179632791_ | 179632791 | 180205791 | rs4700727 | G | A | 0.9483 | −0.0040 | 0.9960 | 0.9924 |
| | A_G | | | | | | | | | |
| 5 | 5_179648650_ | 179648650 | 180221650 | rs139534316 | T | C | 0.0418 | 0.0152 | 1.0153 | 1.0013 |
| | C_T | | | | | | | | | |
| 6 | 6_1106459_ | 1106459 | 1106224 | rs12208662 | A | G | 0.0330 | 0.0171 | 1.0172 | 1.0011 |
| | G_A | | | | | | | | | |
| 6 | 6_1319312_ | 1319312 | 1319077 | rs80199833 | T | C | 0.2001 | −0.0104 | 0.9897 | 0.9959 |
| | C_T | | | | | | | | | |
| 6 | 6_1696841_ | 1696841 | 1696607 | rs11968464 | T | C | 0.1022 | −0.0089 | 0.9911 | 0.9982 |
| | C_T | | | | | | | | | |
| 6 | 6_4400365_ | 4400365 | 4400131 | rs885183 | G | A | 0.3887 | −0.0122 | 0.9879 | 0.9906 |
| | A_G | | | | | | | | | |
| 6 | 6_4445334_ | 4445334 | 4445100 | rs760592016 | T | TTTT | 0.4567 | 0.0019 | 1.0019 | 1.0017 |
| | TTTTC_T | | | | | C | | | | |
| 6 | 6_4456168_ | 4456168 | 4455934 | rs4959980 | A | G | 0.1581 | −0.0026 | 0.9974 | 0.9992 |
| | G_A | | | | | | | | | |
| 6 | 6_4838373_ | 4838373 | 4838139 | rs73364556 | A | G | 0.2172 | −0.0020 | 0.9980 | 0.9991 |
| | G_A | | | | | | | | | |
| 6 | 6_4909673_ | 4909673 | 4909439 | rs9405784 | T | C | 0.7577 | 0.0011 | 1.0011 | 1.0017 |
| | C_T | | | | | | | | | |
| 6 | 6_4915203_ | 4915203 | 4914969 | rs4144546 | T | C | 0.8514 | 0.0079 | 1.0079 | 1.0136 |
| | C_T | | | | | | | | | |
| 6 | 6_4937662_ | 4937662 | 4937428 | rs9502253 | C | A | 0.8536 | 0.0002 | 1.0002 | 1.0003 |
| | A_C | | | | | | | | | |
| 6 | 6_4938570_ | 4938570 | 4938336 | rs4960052 | G | A | 0.9267 | 0.0083 | 1.0083 | 1.0155 |
| | A_G | | | | | | | | | |
| 6 | 6_5275365_ | 5275365 | 5275132 | rs4141760 | G | T | 0.7524 | 0.0034 | 1.0034 | 1.0051 |
| | T_G | | | | | | | | | |
| 6 | 6_6711834_ | 6711834 | 6711601 | rs12206305 | G | A | 0.0671 | −0.0209 | 0.9793 | 0.9972 |
| | A_G | | | | | | | | | |
| 6 | 6_6721361_ | 6721361 | 6721128 | 6:6721361_CA | C | CACA | 0.0332 | 0.0163 | 1.0164 | 1.0011 |
| | CACA_C | | | CA_C | | | | | | |
| 6 | 6_7073433_ | 7073433 | 7073200 | rs575187 | A | C | 0.9342 | 0.0128 | 1.0129 | 1.0242 |
| | C_A | | | | | | | | | |
| 6 | 6_7231843_ | 7231843 | 7231610 | rs9379084 | A | G | 0.1152 | 0.0191 | 1.0193 | 1.0044 |
| | G_A | | | | | | | | | |
| 6 | 6_9390456_ | 9390456 | 9390223 | rs9357170 | C | T | 0.0508 | 0.0025 | 1.0025 | 1.0003 |
| | T_C | | | | | | | | | |
| 6 | 6_9414747_ | 9414747 | 9414514 | rs13437177 | C | T | 0.2409 | −0.0018 | 0.9982 | 0.9991 |
| | T_C | | | | | | | | | |
| 6 | 6_9421174_ | 9421174 | 9420941 | rs7770325 | A | G | 0.0263 | −0.0012 | 0.9988 | 0.9999 |
| | G_A | | | | | | | | | |
| 6 | 6_9435142_ | 9435142 | 9434909 | | CA | C | 0.2130 | −0.0104 | 0.9897 | 0.9956 |
| | C_CA | | | | | | | | | |
| 6 | 6_9462254_ | 9462254 | 9462021 | rs9471534 | G | C | 0.0526 | −0.0052 | 0.9948 | 0.9995 |
| | C_G | | | | | | | | | |
| 6 | 6_9464775_ | 9464775 | 9464542 | rs62395077 | C | T | 0.0615 | 0.0000 | 1.0000 | 1.0000 |
| | T_C | | | | | | | | | |
| 6 | 6_9503805_ | 9503805 | 9503572 | rs9381386 | T | C | 0.4962 | 0.0040 | 1.0040 | 1.0040 |
| | C_T | | | | | | | | | |
| 6 | 6_9509256_ | 9509256 | 9509023 | rs114190032 | T | G | 0.4109 | 0.0023 | 1.0023 | 1.0019 |
| | G_T | | | | | | | | | |
| 6 | 6_10130989_ | 10130989 | 10130756 | rs12529019 | T | C | 0.2042 | 0.0016 | 1.0016 | 1.0007 |
| | C_T | | | | | | | | | |
| 6 | 6_10331162_ | 10331162 | 10330929 | rs9350313 | G | C | 0.0787 | −0.0097 | 0.9903 | 0.9985 |
| | C_G | | | | | | | | | |
| 6 | 6_10887932_ | 10887932 | 10887699 | rs2153159 | T | G | 0.4902 | 0.0004 | 1.0004 | 1.0004 |
| | G_T | | | | | | | | | |
| 6 | 6_10891651_ | 10891651 | 10891418 | rs146072680 | A | ATT | 0.8199 | −0.0013 | 0.9987 | 0.9979 |
| | ATT_A | | | | | | | | | |
| 6 | 6_12261921_ | 12261921 | 12261688 | rs6458155 | C | T | 0.3350 | −0.0031 | 0.9969 | 0.9979 |
| | T_C | | | | | | | | | |
| 6 | 6_12448424_ | 12448424 | 12448192 | rs190929774 | T | C | 0.0126 | −0.0167 | 0.9834 | 0.9996 |
| | C_T | | | | | | | | | |
| 6 | 6_12860700_ | 12860700 | 12860468 | rs6924353 | T | C | 0.0664 | −0.0068 | 0.9932 | 0.9991 |
| | C_T | | | | | | | | | |
| 6 | 6_13192799_ | 13192799 | 13192567 | rs4273688 | A | G | 0.1629 | 0.0115 | 1.0116 | 1.0038 |
| | G_A | | | | | | | | | |
| 6 | 6_13600459_ | 13600459 | 13600227 | rs2253217 | C | T | 0.3137 | 0.0000 | 1.0000 | 1.0000 |
| | T_C | | | | | | | | | |
| 6 | 6_13642999_ | 13642999 | 13642767 | rs3752501 | C | T | 0.3107 | −0.0045 | 0.9955 | 0.9972 |
| | T_C | | | | | | | | | |
| 6 | 6_13700719_ | 13700719 | 13700487 | 6:13700719_A | A | AGTA | 0.3723 | −0.0128 | 0.9873 | 0.9906 |
| | AGTAG_A | | | GTAG_A | | G | | | | |
| 6 | 6_13713366_ | 13713366 | 13713134 | rs418053 | C | G | 0.5691 | −0.0092 | 0.9908 | 0.9896 |
| | G_C | | | | | | | | | |
| 6 | 6_13719673_ | 13719673 | 13719441 | rs412970 | A | G | 0.4909 | 0.0000 | 1.0000 | 1.0000 |
| | G_A | | | | | | | | | |
| 6 | 6_13741516_ | 13741516 | 13741284 | rs376024 | C | T | 0.5760 | −0.0112 | 0.9889 | 0.9872 |
| | T_C | | | | | | | | | |
| 6 | 6_14288962_ | 14288962 | 14288731 | rs71552783 | TGAA | T | 0.0835 | 0.0034 | 1.0034 | 1.0006 |
| | T_TGAATCA | | | | TCA | | | | | |
| 6 | 6_14657742_ | 14657742 | 14657511 | rs10484340 | A | G | 0.4154 | 0.0103 | 1.0104 | 1.0086 |
| | G_A | | | | | | | | | |
| 6 | 6_14665517_ | 14665517 | 14665286 | rs2092918 | C | T | 0.3769 | 0.0014 | 1.0014 | 1.0011 |
| | T_C | | | | | | | | | |
| 6 | 6_14668667_ | 14668667 | 14668436 | rs7761066 | G | A | 0.5444 | 0.0000 | 1.0000 | 1.0000 |
| | A_G | | | | | | | | | |
| 6 | 6_14892230_ | 14892230 | 14891999 | rs837181 | A | G | 0.7348 | 0.0074 | 1.0074 | 1.0109 |
| | G_A | | | | | | | | | |
| 6 | 6_14912686_ | 14912686 | 14912455 | rs439819 | A | G | 0.7488 | 0.0023 | 1.0023 | 1.0035 |
| | G_A | | | | | | | | | |
| 6 | 6_16399557_ | 16399557 | 16399326 | rs3819405 | T | C | 0.3299 | −0.0199 | 0.9803 | 0.9870 |
| | C_T | | | | | | | | | |
| 6 | 6_17096277_ | 17096277 | 17096046 | rs74443408 | T | G | 0.1208 | −0.0141 | 0.9860 | 0.9966 |
| | G_T | | | | | | | | | |
| 6 | 6_18520529_ | 18520529 | 18520298 | rs66680090 | G | A | 0.2582 | 0.0086 | 1.0086 | 1.0045 |
| | A_G | | | | | | | | | |
| 6 | 6_18757303_ | 18757303 | 18757072 | rs556702310 | TA | T | 0.4562 | 0.0000 | 1.0000 | 1.0000 |
| | T_TA | | | | | | | | | |
| 6 | 6_18783140_ | 18783140 | 18782909 | rs12211970 | A | G | 0.6200 | 0.0071 | 1.0071 | 1.0089 |
| | G_A | | | | | | | | | |
| 6 | 6_18786308_ | 18786308 | 18786077 | 6:18786308_C | C | CCTT | 0.6704 | 0.0009 | 1.0009 | 1.0012 |
| | CCTTCCCTTC | | | CTTCCCTTCT_ | | CCCT | | | | |
| | T_C | | | C | | TCT | | | | |
| 6 | 6_19051225_ | 19051225 | 19050994 | rs1207107 | A | C | 0.1204 | −0.0012 | 0.9988 | 0.9997 |
| | C_A | | | | | | | | | |
| 6 | 6_20537845_ | 20537845 | 20537614 | rs769485514 | C | CA | 0.4733 | −0.0080 | 0.9920 | 0.9925 |
| | CA_C | | | | | | | | | |
| 6 | 6_20634428_ | 20634428 | 20634197 | rs9465847 | T | G | 0.1747 | 0.0000 | 1.0000 | 1.0000 |
| | G_T | | | | | | | | | |
| 6 | 6_20639604_ | 20639604 | 20639373 | rs7758612 | A | G | 0.6946 | −0.0032 | 0.9968 | 0.9956 |
| | G_A | | | | | | | | | |
| 6 | 6_20645999_ | 20645999 | 20645768 | 6:20645999_A | A | AATT | 0.5640 | 0.0000 | 1.0000 | 1.0000 |
| | AATTTT_A | | | ATTTT_A | | TT | | | | |
| 6 | 6_20666919_ | 20666919 | 20666688 | 6:20666919_C | C | CT | 0.6369 | −0.0005 | 0.9995 | 0.9994 |
| | CT_C | | | T_C | | | | | | |
| 6 | 6_20687802_ | 20687802 | 20687571 | rs10440832 | A | G | 0.8056 | −0.0045 | 0.9955 | 0.9928 |
| | G_A | | | | | | | | | |
| 6 | 6_20706418_ | 20706418 | 20706187 | rs75626419 | T | C | 0.1159 | −0.0088 | 0.9912 | 0.9980 |
| | C_T | | | | | | | | | |
| 6 | 6_20736237_ | 20736237 | 20736006 | rs9358361 | A | C | 0.8370 | −0.0010 | 0.9990 | 0.9983 |
| | C_A | | | | | | | | | |
| 6 | 6_20830700_ | 20830700 | 20830469 | 6:20830700_G | G | GAT | 0.1919 | 0.0034 | 1.0034 | 1.0013 |
| | GAT_G | | | AT_G | | | | | | |
| 6 | 6_20899840_ | 20899840 | 20899609 | rs12154181 | T | C | 0.2916 | 0.0021 | 1.0021 | 1.0012 |
| | C_T | | | | | | | | | |
| 6 | 6_20922863_ | 20922863 | 20922632 | rs9460577 | T | C | 0.2856 | −0.0031 | 0.9969 | 0.9982 |
| | C_T | | | | | | | | | |
| 6 | 6_21905726_ | 21905726 | 21905495 | rs9350395 | G | A | 0.5920 | 0.0000 | 1.0000 | 1.0000 |
| | A_G | | | | | | | | | |
| 6 | 6_21910779_ | 21910779 | 21910548 | rs138872161 | T | C | 0.3467 | −0.0017 | 0.9983 | 0.9988 |
| | C_T | | | | | | | | | |
| 6 | 6_21923810_ | 21923810 | 21923579 | rs9358466 | C | T | 0.4303 | −0.0006 | 0.9994 | 0.9995 |
| | T_C | | | | | | | | | |
| 6 | 6_21925227_ | 21925227 | 21924996 | rs9358467 | A | T | 0.4466 | −0.0103 | 0.9898 | 0.9909 |
| | T_A | | | | | | | | | |
| 6 | 6_21948207_ | 21948207 | 21947976 | rs10946491 | C | T | 0.2594 | −0.0003 | 0.9997 | 0.9998 |
| | T_C | | | | | | | | | |
| 6 | 6_21967114_ | 21967114 | 21966883 | rs79266217 | T | C | 0.2401 | −0.0043 | 0.9957 | 0.9979 |
| | C_T | | | | | | | | | |
| 6 | 6_22220514_ | 22220514 | 22220285 | rs74371547 | A | G | 0.0166 | 0.0265 | 1.0269 | 1.0009 |
| | G_A | | | | | | | | | |
| 6 | 6_22968438_ | 22968438 | 22968209 | rs112016347 | C | T | 0.0539 | −0.0106 | 0.9895 | 0.9989 |
| | T_C | | | | | | | | | |
| 6 | 6_23019927_ | 23019927 | 23019698 | rs188165595 | A | C | 0.0148 | −0.0129 | 0.9872 | 0.9996 |
| | C_A | | | | | | | | | |
| 6 | 6_23446154_ | 23446154 | 23445926 | rs12529389 | A | G | 0.2515 | −0.0103 | 0.9898 | 0.9949 |
| | G_A | | | | | | | | | |
| 6 | 6_25148056_ | 25148056 | 25147828 | rs77078042 | T | C | 0.0388 | −0.0018 | 0.9982 | 0.9999 |
| | C_T | | | | | | | | | |
| 6 | 6_26759078_ | 26759078 | 26742807 | rs13195977 | A | C | 0.1368 | −0.0010 | 0.9990 | 0.9997 |
| | C_A | | | | | | | | | |
| 6 | 6_27274834_ | 27274834 | 27307055 | rs56114371 | T | C | 0.0841 | −0.0016 | 0.9984 | 0.9997 |
| | C_T | | | | | | | | | |
| 6 | 6_27425644_ | 27425644 | 27457865 | rs34196306 | C | G | 0.0815 | −0.0251 | 0.9752 | 0.9960 |
| | G_C | | | | | | | | | |
| 6 | 6_27848882_ | 27848882 | 27881104 | rs72847368 | C | T | 0.0939 | 0.0056 | 1.0056 | 1.0011 |
| | T_C | | | | | | | | | |
| 6 | 6_28117602_ | 28117602 | 28149824 | rs560097346 | CT | C | 0.1351 | 0.0080 | 1.0080 | 1.0022 |
| | C_CT | | | | | | | | | |
| 6 | 6_28897567_ | 28897567 | 28929790 | 6:28897567_T | T | TA | 0.3455 | 0.0102 | 1.0103 | 1.0071 |
| | TA_T | | | A_T | | | | | | |
| 6 | 6_29369052_ | 29369052 | 29401275 | rs113838413 | A | G | 0.0212 | 0.0174 | 1.0176 | 1.0007 |
| | G_A | | | | | | | | | |
| 6 | 6_29530628_ | 29530628 | 29562851 | rs362512 | G | A | 0.5050 | 0.0005 | 1.0005 | 1.0005 |
| | A_G | | | | | | | | | |
| 6 | 6_29861210_ | 29861210 | 29893433 | | A | G | 0.0257 | −0.0210 | 0.9792 | 0.9989 |
| | G_A | | | | | | | | | |
| 6 | 6_30454350_ | 30454350 | 30486573 | rs17189022 | A | G | 0.0178 | 0.0000 | 1.0000 | 1.0000 |
| | G_A | | | | | | | | | |
| 6 | 6_31093776_ | 31093776 | 31125999 | rs3778639 | G | A | 0.1049 | 0.0000 | 1.0000 | 1.0000 |
| | A_G | | | | | | | | | |
| 6 | 6_31096526_ | 31096526 | 31128749 | rs9263717 | G | A | 0.2188 | 0.0018 | 1.0018 | 1.0008 |
| | A_G | | | | | | | | | |
| 6 | 6_31274174_ | 31274174 | 31306397 | rs139101653 | T | C | 0.0238 | −0.0150 | 0.9851 | 0.9993 |
| | C_T | | | | | | | | | |
| 6 | 6_32583605_ | 32583605 | 32615828 | rs34341095 | G | A | 0.4400 | −0.0002 | 0.9998 | 0.9998 |
| | A_G | | | | | | | | | |
| 6 | 6_32583813_ | 32583813 | 32616036 | rs1846190 | A | G | 0.2972 | −0.0071 | 0.9929 | 0.9958 |
| | G_A | | | | | | | | | |
| 6 | 6_32584313_ | 32584313 | 32616536 | rs33932178 | G | C | 0.3290 | −0.0015 | 0.9985 | 0.9990 |
| | C_G | | | | | | | | | |
| 6 | 6_32600671_ | 32600671 | 32632894 | rs9272131 | T | C | 0.0972 | −0.0068 | 0.9932 | 0.9987 |
| | C_T | | | | | | | | | |
| 6 | 6_32693326_ | 32693326 | 32725549 | rs112269866 | G | A | 0.6904 | 0.0025 | 1.0025 | 1.0035 |
| | A_G | | | | | | | | | |
| 6 | 6_33239869_ | 33239869 | 33272092 | rs17215231 | T | C | 0.0795 | 0.0000 | 1.0000 | 1.0000 |
| | C_T | | | | | | | | | |
| 6 | 6_33249617_ | 33249617 | 33281840 | 6:33249617_C | C | CCTT | 0.3539 | 0.0000 | 1.0000 | 1.0000 |
| | CCTTTT_C | | | CTTTT_C | | TT | | | | |
| 6 | 6_33820658_ | 33820658 | 33852881 | rs78861422 | T | C | 0.0349 | 0.0164 | 1.0165 | 1.0012 |
| | C_T | | | | | | | | | |
| 6 | 6_33822865_ | 33822865 | 33855088 | rs9296100 | T | C | 0.7697 | −0.0120 | 0.9881 | 0.9817 |
| | C_T | | | | | | | | | |
| 6 | 6_34833044_ | 34833044 | 34865267 | rs35173014 | T | C | 0.0132 | −0.0245 | 0.9758 | 0.9994 |
| | C_T | | | | | | | | | |
| 6 | 6_35998776_ | 35998776 | 36030999 | rs13196204 | G | T | 0.1768 | −0.0056 | 0.9944 | 0.9980 |
| | T_G | | | | | | | | | |
| 6 | 6_36641993_ | 36641993 | 36674216 | rs148260186 | A | C | 0.0250 | 0.0112 | 1.0113 | 1.0006 |
| | C_A | | | | | | | | | |
| 6 | 6_36644498_ | 36644498 | 36676721 | rs3829964 | C | T | 0.4919 | 0.0125 | 1.0126 | 1.0124 |
| | T_C | | | | | | | | | |
| 6 | 6_37699029_ | 37699029 | 37731253 | rs13218687 | T | C | 0.1384 | −0.0054 | 0.9946 | 0.9985 |
| | C_T | | | | | | | | | |
| 6 | 6_42078871_ | 42078871 | 42111133 | rs4357133 | T | G | 0.3103 | 0.0054 | 1.0054 | 1.0034 |
| | G_T | | | | | | | | | |
| 6 | 6_43227141_ | 43227141 | 43259403 | rs111342015 | A | G | 0.0985 | −0.0245 | 0.9758 | 0.9952 |
| | G_A | | | | | | | | | |
| 6 | 6_43757082_ | 43757082 | 43789345 | rs4711750 | A | T | 0.4897 | −0.0096 | 0.9904 | 0.9907 |
| | T_A | | | | | | | | | |
| 6 | 6_45892162_ | 45892162 | 45924425 | rs4714889 | A | G | 0.9692 | −0.0348 | 0.9658 | 0.9348 |
| | G_A | | | | | | | | | |
| 6 | 6_46877011_ | 46877011 | 46909274 | rs1328958 | A | T | 0.1356 | −0.0031 | 0.9969 | 0.9992 |
| | T_A | | | | | | | | | |
| 6 | 6_46889321_ | 46889321 | 46921584 | rs76199887 | A | C | 0.0702 | −0.0188 | 0.9814 | 0.9974 |
| | C_A | | | | | | | | | |
| 6 | 6_46897275_ | 46897275 | 46929538 | 6:46897275_G | G | GA | 0.7599 | 0.0009 | 1.0009 | 1.0014 |
| | GA_G | | | A_G | | | | | | |
| 6 | 6_47336402_ | 47336402 | 47368666 | 6:47336402_T | T | TTTT | 0.4523 | 0.0000 | 1.0000 | 1.0000 |
| | TTTTATA_T | | | TTTATA_T | | ATA | | | | |
| 6 | 6_47898772_ | 47898772 | 47931036 | rs149063534 | T | C | 0.0286 | 0.0061 | 1.0061 | 1.0004 |
| | C_T | | | | | | | | | |
| 6 | 6_52205511_ | 52205511 | 52340713 | rs7774765 | T | G | 0.0548 | 0.0072 | 1.0072 | 1.0008 |
| | G_T | | | | | | | | | |
| 6 | 6_52980128_ | 52980128 | 53115330 | rs6941531 | T | C | 0.0320 | −0.0140 | 0.9861 | 0.9991 |
| | C_T | | | | | | | | | |
| 6 | 6_52984063_ | 52984063 | 53119265 | rs9474392 | A | C | 0.0175 | −0.0313 | 0.9692 | 0.9989 |
| | C_A | | | | | | | | | |
| 6 | 6_54005663_ | 54005663 | 54140865 | rs816374 | G | A | 0.2890 | −0.0046 | 0.9954 | 0.9973 |
| | A_G | | | | | | | | | |
| 6 | 6_54700803_ | 54700803 | 54836005 | rs1393779 | C | G | 0.4905 | 0.0009 | 1.0009 | 1.0009 |
| | G_C | | | | | | | | | |
| 6 | 6_54714635_ | 54714635 | 54849837 | rs9464149 | G | T | 0.4778 | 0.0001 | 1.0001 | 1.0001 |
| | T_G | | | | | | | | | |
| 6 | 6_54726869_ | 54726869 | 54862071 | rs931766 | T | C | 0.5053 | 0.0089 | 1.0089 | 1.0091 |
| | C_T | | | | | | | | | |
| 6 | 6_54800275_ | 54800275 | 54935477 | rs56233640 | G | A | 0.2546 | 0.0026 | 1.0026 | 1.0013 |
| | A_G | | | | | | | | | |
| 6 | 6_55089182_ | 55089182 | 55224384 | rs10948897 | A | G | 0.5158 | 0.0001 | 1.0001 | 1.0001 |
| | G_A | | | | | | | | | |
| 6 | 6_61948121_ | 61948121 | 61289594 | rs569725482 | T | C | 0.0106 | 0.0381 | 1.0388 | 1.0008 |
| | C_T | | | | | | | | | |
| 6 | 6_61972502_ | 61972502 | 61265212 | rs12197273 | C | T | 0.2801 | 0.0006 | 1.0006 | 1.0003 |
| | T_C | | | | | | | | | |
| 6 | 6_64505213_ | 64505213 | 63795320 | rs756274 | G | A | 0.3466 | 0.0008 | 1.0008 | 1.0006 |
| | A_G | | | | | | | | | |
| 6 | 6_68849351_ | 68849351 | 68139459 | rs77203199 | G | T | 0.1700 | 0.0013 | 1.0013 | 1.0004 |
| | T_G | | | | | | | | | |
| 6 | 6_69273849_ | 69273849 | 68563957 | rs62416495 | G | A | 0.0217 | −0.0075 | 0.9925 | 0.9997 |
| | A_G | | | | | | | | | |
| 6 | 6_69300758_ | 69300758 | 68590866 | rs117298640 | T | C | 0.0209 | 0.0236 | 1.0239 | 1.0010 |
| | C_T | | | | | | | | | |
| 6 | 6_72994975_ | 72994975 | 72285272 | rs10455087 | T | C | 0.1095 | 0.0036 | 1.0036 | 1.0008 |
| | C_T | | | | | | | | | |
| 6 | 6_73138812_ | 73138812 | 72429110 | rs12196354 | A | G | 0.1700 | 0.0091 | 1.0091 | 1.0031 |
| | G_A | | | | | | | | | |
| 6 | 6_73157462_ | 73157462 | 72447760 | rs6927917 | G | A | 0.8400 | −0.0035 | 0.9965 | 0.9941 |
| | A_G | | | | | | | | | |
| 6 | 6_73575924_ | 73575924 | 72866201 | rs2995345 | C | T | 0.7807 | 0.0106 | 1.0107 | 1.0167 |
| | T_C | | | | | | | | | |
| 6 | 6_73588636_ | 73588636 | 72878913 | rs9442863 | G | T | 0.2119 | −0.0067 | 0.9933 | 0.9972 |
| | T_G | | | | | | | | | |
| 6 | 6_74819460_ | 74819460 | 74109744 | rs55976071 | A | G | 0.1525 | −0.0095 | 0.9905 | 0.9971 |
| | G_A | | | | | | | | | |
| 6 | 6_76267791_ | 76267791 | 75558075 | 6:76267791_A | A | AT | 0.5132 | −0.0048 | 0.9952 | 0.9951 |
| | AT_A | | | T_A | | | | | | |
| 6 | 6_78924045_ | 78924045 | 78214328 | rs867705 | T | A | 0.3338 | −0.0015 | 0.9985 | 0.9990 |
| | A_T | | | | | | | | | |
| 6 | 6_79037890_ | 79037890 | 78328173 | rs611413 | G | A | 0.1614 | −0.0095 | 0.9905 | 0.9970 |
| | A_G | | | | | | | | | |
| 6 | 6_81105862_ | 81105862 | 80396145 | rs1836962 | G | T | 0.5419 | 0.0014 | 1.0014 | 1.0015 |
| | T_G | | | | | | | | | |
| 6 | 6_81193866_ | 81193866 | 80484149 | rs2917619 | C | T | 0.4972 | −0.0030 | 0.9970 | 0.9970 |
| | T_C | | | | | | | | | |
| 6 | 6_81306990_ | 81306990 | 80597273 | rs7750052 | T | A | 0.4540 | −0.0018 | 0.9982 | 0.9984 |
| | A_T | | | | | | | | | |
| 6 | 6_81974654_ | 81974654 | 81264937 | rs75640685 | G | A | 0.0564 | 0.0000 | 1.0000 | 1.0000 |
| | A_G | | | | | | | | | |
| 6 | 6_82226268_ | 82226268 | 81516551 | rs117486497 | A | T | 0.0542 | 0.0089 | 1.0089 | 1.0010 |
| | T_A | | | | | | | | | |
| 6 | 6_82263549_ | 82263549 | 81553832 | rs10623112 | A | AAT | 0.4262 | 0.0137 | 1.0138 | 1.0118 |
| | AATA | | | | | | | | | |
| 6 | 6_82280232_ | 82280232 | 81570515 | rs7771821 | A | T | 0.2552 | 0.0046 | 1.0046 | 1.0024 |
| | T_A | | | | | | | | | |
| 6 | 6_82287685_ | 82287685 | 81577968 | rs1361549 | A | G | 0.4150 | −0.0100 | 0.9900 | 0.9918 |
| | G_A | | | | | | | | | |
| 6 | 6_82725389_ | 82725389 | 82015672 | rs4706918 | C | T | 0.6471 | 0.0000 | 1.0000 | 1.0000 |
| | T_C | | | | | | | | | |
| 6 | 6_85102918_ | 85102918 | 84393200 | rs62448657 | A | G | 0.1014 | 0.0070 | 1.0070 | 1.0014 |
| | G_A | | | | | | | | | |
| 6 | 6_85689822_ | 85689822 | 84980104 | rs56413943 | G | A | 0.1432 | 0.0007 | 1.0007 | 1.0002 |
| | A_G | | | | | | | | | |
| 6 | 6_85722437_ | 85722437 | 85012719 | rs62422908 | C | T | 0.1067 | 0.0152 | 1.0153 | 1.0033 |
| | T_C | | | | | | | | | |
| 6 | 6_85746200_ | 85746200 | 85036482 | rs142990780 | A | C | 0.1006 | 0.0050 | 1.0050 | 1.0010 |
| | C_A | | | | | | | | | |
| 6 | 6_85991348_ | 85991348 | 85281630 | rs9450216 | T | A | 0.0803 | 0.0017 | 1.0017 | 1.0003 |
| | A_T | | | | | | | | | |
| 6 | 6_86913286_ | 86913286 | 86203568 | rs62443510 | A | T | 0.0927 | −0.0014 | 0.9986 | 0.9997 |
| | T_A | | | | | | | | | |
| 6 | 6_86973861_ | 86973861 | 86264143 | 6:86973861_T | T | TC | 0.0993 | −0.0143 | 0.9858 | 0.9972 |
| | TC_T | | | C_T | | | | | | |
| 6 | 6_87648885_ | 87648885 | 86939167 | rs862420 | G | C | 0.1378 | 0.0026 | 1.0026 | 1.0007 |
| | C_G | | | | | | | | | |
| 6 | 6_87803819_ | 87803819 | 87094101 | rs73754909 | C | T | 0.2770 | 0.0128 | 1.0129 | 1.0072 |
| | T_C | | | | | | | | | |
| 6 | 6_87834796_ | 87834796 | 87125078 | rs11753770 | G | A | 0.0984 | 0.0029 | 1.0029 | 1.0006 |
| | A_G | | | | | | | | | |
| 6 | 6_90141980_ | 90141980 | 89432261 | rs56170939 | G | A | 0.1511 | 0.0147 | 1.0148 | 1.0045 |
| | A_G | | | | | | | | | |
| 6 | 6_90474544_ | 90474544 | 89764825 | rs9359863 | A | G | 0.0524 | −0.0012 | 0.9988 | 0.9999 |
| | G_A | | | | | | | | | |
| 6 | 6_97219145_ | 97219145 | 96771269 | rs780491270 | C | CA | 0.2187 | 0.0000 | 1.0000 | 1.0000 |
| | CA_C | | | | | | | | | |
| 6 | 6_98462501_ | 98462501 | 98014625 | rs12529874 | A | G | 0.0345 | −0.0111 | 0.9890 | 0.9992 |
| | G_A | | | | | | | | | |
| 6 | 6_100048974_ | 100048974 | 99601098 | rs9385807 | C | T | 0.1373 | 0.0030 | 1.0030 | 1.0008 |
| | T_C | | | | | | | | | |
| 6 | 6_100091541_ | 100091541 | 99643665 | 6:100091541_ | C | CAAA | 0.2287 | 0.0026 | 1.0026 | 1.0012 |
| | CAAA_C | | | CAAA_C | | | | | | |
| 6 | 6_100184696_ | 100184696 | 99736820 | rs6929136 | G | A | 0.2023 | 0.0028 | 1.0028 | 1.0011 |
| | A_G | | | | | | | | | |
| 6 | 6_100317114_ | 100317114 | 99869238 | rs6916454 | A | G | 0.3486 | 0.0051 | 1.0051 | 1.0036 |
| | G_A | | | | | | | | | |
| 6 | 6_104762291_ | 104762291 | 104314416 | rs905538 | A | T | 0.3253 | 0.0107 | 1.0108 | 1.0070 |
| | T_A | | | | | | | | | |
| 6 | 6_110776061_ | 110776061 | 110454858 | 6:110776061_ | A | ATGT | 0.0523 | −0.0243 | 0.9760 | 0.9975 |
| | ATGT_A | | | ATGT_A | | | | | | |
| 6 | 6_111236431_ | 111236431 | 110915228 | rs13203604 | A | G | 0.1641 | −0.0064 | 0.9936 | 0.9979 |
| | G_A | | | | | | | | | |
| 6 | 6_111313720_ | 111313720 | 110992517 | rs13203579 | G | A | 0.2391 | −0.0051 | 0.9949 | 0.9976 |
| | A_G | | | | | | | | | |
| 6 | 6_111492119_ | 111492119 | 111170916 | rs241768 | C | T | 0.7090 | −0.0045 | 0.9955 | 0.9936 |
| | T_C | | | | | | | | | |
| 6 | 6_111947938_ | 111947938 | 111626735 | rs117553096 | G | A | 0.0257 | −0.0287 | 0.9717 | 0.9985 |
| | A_G | | | | | | | | | |
| 6 | 6_112830623_ | 112830623 | 112509421 | rs147637256 | T | C | 0.0279 | −0.0170 | 0.9831 | 0.9991 |
| | C_T | | | | | | | | | |
| 6 | 6_113986557_ | 113986557 | 113665355 | 6:113986557_ | A | ATT | 0.6740 | 0.0038 | 1.0038 | 1.0051 |
| | ATT_A | | | ATT_A | | | | | | |
| 6 | 6_114387136_ | 114387136 | 114065972 | rs142313879 | T | C | 0.0159 | −0.0125 | 0.9876 | 0.9996 |
| | C_T | | | | | | | | | |
| 6 | 6_115102204_ | 115102204 | 114781040 | rs62442934 | C | A | 0.1357 | −0.0218 | 0.9784 | 0.9942 |
| | A_C | | | | | | | | | |
| 6 | 6_118214194_ | 118214194 | 117893031 | rs56013995 | C | G | 0.1824 | 0.0043 | 1.0043 | 1.0016 |
| | G_C | | | | | | | | | |
| 6 | 6_119578767_ | 119578767 | 119257602 | rs147917246 | G | A | 0.0128 | 0.0330 | 1.0336 | 1.0009 |
| | A_G | | | | | | | | | |
| 6 | 6_120623111_ | 120623111 | 120301965 | rs66945651 | C | T | 0.5301 | −0.0076 | 0.9924 | 0.9920 |
| | T_C | | | | | | | | | |
| 6 | 6_122329970_ | 122329970 | 122008824 | rs2684222 | A | C | 0.7019 | 0.0102 | 1.0103 | 1.0144 |
| | C_A | | | | | | | | | |
| 6 | 6_122625590_ | 122625590 | 122304445 | rs13191873 | A | G | 0.0596 | −0.0232 | 0.9771 | 0.9973 |
| | G_A | | | | | | | | | |
| 6 | 6_122717777_ | 122717777 | 122396632 | rs76071582 | T | C | 0.1109 | 0.0051 | 1.0051 | 1.0011 |
| | C_T | | | | | | | | | |
| 6 | 6_122719631_ | 122719631 | 122398486 | rs516805 | T | G | 0.7003 | −0.0074 | 0.9926 | 0.9897 |
| | G_T | | | | | | | | | |
| 6 | 6_123366449_ | 123366449 | 123045304 | rs6940619 | T | A | 0.2294 | −0.0003 | 0.9997 | 0.9999 |
| | A_T | | | | | | | | | |
| 6 | 6_123825415_ | 123825415 | 123504270 | rs7746287 | G | T | 0.8692 | −0.0071 | 0.9929 | 0.9877 |
| | T_G | | | | | | | | | |
| 6 | 6_124613800_ | 124613800 | 124292654 | 6:124613800_ | T | TTAC | 0.1758 | 0.0088 | 1.0088 | 1.0031 |
| | TTACATTGA_T | | | TTACATTGA_T | | ATTG | | | | |
| | | | | | | A | | | | |
| 6 | 6_125172405_ | 125172405 | 124851259 | rs1409293 | A | G | 0.4874 | −0.0049 | 0.9951 | 0.9952 |
| | G_A | | | | | | | | | |
| 6 | 6_125223597_ | 125223597 | 124902451 | rs11154277 | G | T | 0.3993 | −0.0079 | 0.9921 | 0.9937 |
| | T_G | | | | | | | | | |
| 6 | 6_127457071_ | 127457071 | 127135926 | 6:127457071_ | C | CA | 0.5389 | −0.0013 | 0.9987 | 0.9986 |
| | CA_C | | | CA_C | | | | | | |
| 6 | 6_127477021_ | 127477021 | 127155876 | rs139087942 | GTAT | G | 0.4906 | −0.0058 | 0.9942 | 0.9943 |
| | G_GTATA | | | | A | | | | | |
| 6 | 6_129916283_ | 129916283 | 129595138 | rs10633215 | CAT | C | 0.4745 | 0.0000 | 1.0000 | 1.0000 |
| | C_CAT | | | | | | | | | |
| 6 | 6_129922776_ | 129922776 | 129601631 | rs34417139 | CT | C | 0.3343 | −0.0028 | 0.9972 | 0.9981 |
| | C_CT | | | | | | | | | |
| 6 | 6_130341235_ | 130341235 | 130020090 | rs113898003 | C | T | 0.2562 | 0.0051 | 1.0051 | 1.0026 |
| | T_C | | | | | | | | | |
| 6 | 6_130341728_ | 130341728 | 130020583 | rs55941023 | CT | C | 0.7116 | 0.0119 | 1.0120 | 1.0171 |
| | C_CT | | | | | | | | | |
| 6 | 6_130349119_ | 130349119 | 130027974 | rs6569648 | T | C | 0.7627 | 0.0174 | 1.0176 | 1.0270 |
| | C_T | | | | | | | | | |
| 6 | 6_130436472_ | 130436472 | 130115327 | rs6914419 | T | C | 0.3643 | 0.0000 | 1.0000 | 1.0000 |
| | C_T | | | | | | | | | |
| 6 | 6_132375163_ | 132375163 | 132054023 | 6:132375163_ | A | AT | 0.6028 | 0.0018 | 1.0018 | 1.0022 |
| | AT_A | | | AT_A | | | | | | |
| 6 | 6_133703325_ | 133703325 | 133382187 | rs2235485 | G | A | 0.1475 | 0.0119 | 1.0120 | 1.0035 |
| | A_G | | | | | | | | | |
| 6 | 6_135262565_ | 135262565 | 134941427 | rs13209279 | A | G | 0.3406 | 0.0070 | 1.0070 | 1.0048 |
| | G_A | | | | | | | | | |
| 6 | 6_135457541_ | 135457541 | 135136403 | rs1883354 | G | A | 0.4182 | 0.0016 | 1.0016 | 1.0013 |
| | A_G | | | | | | | | | |
| 6 | 6_135492534_ | 135492534 | 135171396 | rs6903653 | T | C | 0.3871 | 0.0046 | 1.0046 | 1.0036 |
| | C_T | | | | | | | | | |
| 6 | 6_135646235_ | 135646235 | 135325097 | rs9402703 | G | A | 0.5649 | 0.0093 | 1.0093 | 1.0106 |
| | A_G | | | | | | | | | |
| 6 | 6_138834164_ | 138834164 | 138513027 | rs6570241 | C | T | 0.0762 | 0.0000 | 1.0000 | 1.0000 |
| | T_C | | | | | | | | | |
| 6 | 6_139134041_ | 139134041 | 138812904 | rs9403003 | C | G | 0.2660 | −0.0105 | 0.9896 | 0.9945 |
| | G_C | | | | | | | | | |
| 6 | 6_139153321_ | 139153321 | 138832184 | | G | GTT | 0.1000 | −0.0121 | 0.9880 | 0.9976 |
| | GTT_G | | | | | | | | | |
| 6 | 6_139603414_ | 139603414 | 139282277 | rs9495413 | A | G | 0.2454 | 0.0099 | 1.0099 | 1.0049 |
| | G_A | | | | | | | | | |
| 6 | 6_144311702_ | 144311702 | 143990565 | rs76161579 | T | C | 0.0108 | −0.0077 | 0.9923 | 0.9998 |
| | C_T | | | | | | | | | |
| 6 | 6_149595505_ | 149595505 | 149274369 | rs2121348 | C | T | 0.2061 | −0.0230 | 0.9773 | 0.9906 |
| | T_C | | | | | | | | | |
| 6 | 6_149601768_ | 149601768 | 149280632 | rs4897108 | A | G | 0.2309 | 0.0000 | 1.0000 | 1.0000 |
| | G_A | | | | | | | | | |
| 6 | 6_149770179_ | 149770179 | 149449043 | rs237005 | T | C | 0.2512 | 0.0043 | 1.0043 | 1.0022 |
| | C_T | | | | | | | | | |
| 6 | 6_150576958_ | 150576958 | 150255822 | rs111766406 | A | G | 0.0140 | 0.0000 | 1.0000 | 1.0000 |
| | G_A | | | | | | | | | |
| 6 | 6_150587307_ | 150587307 | 150266171 | rs6936991 | C | T | 0.0270 | 0.0121 | 1.0122 | 1.0007 |
| | T_C | | | | | | | | | |
| 6 | 6_150599606_ | 150599606 | 150278470 | rs12214271 | T | C | 0.1402 | −0.0075 | 0.9925 | 0.9979 |
| | C_T | | | | | | | | | |
| 6 | 6_150807661_ | 150807661 | 150486525 | rs9480463 | G | A | 0.5128 | 0.0017 | 1.0017 | 1.0017 |
| | A_G | | | | | | | | | |
| 6 | 6_150812557_ | 150812557 | 150491421 | rs12207876 | A | T | 0.1023 | −0.0020 | 0.9980 | 0.9996 |
| | T_A | | | | | | | | | |
| 6 | 6_150894274_ | 150894274 | 150573138 | rs9480482 | A | G | 0.1844 | −0.0052 | 0.9948 | 0.9981 |
| | G_A | | | | | | | | | |
| 6 | 6_151214063_ | 151214063 | 150892927 | 6:151214063_ | T | TG | 0.1010 | −0.0121 | 0.9880 | 0.9976 |
| | TG_T | | | TG_T | | | | | | |
| 6 | 6_151371751_ | 151371751 | 151050615 | rs6928026 | A | G | 0.4760 | −0.0030 | 0.9970 | 0.9972 |
| | G_A | | | | | | | | | |
| 6 | 6_151419174_ | 151419174 | 151098038 | rs9478949 | G | A | 0.3867 | 0.0052 | 1.0052 | 1.0040 |
| | A_G | | | | | | | | | |
| 6 | 6_151932778_ | 151932778 | 151611643 | rs1023940 | G | A | 0.4960 | 0.0000 | 1.0000 | 1.0000 |
| | A_G | | | | | | | | | |
| 6 | 6_151948366_ | 151948366 | 151627231 | rs2046210 | A | G | 0.3403 | 0.0000 | 1.0000 | 1.0000 |
| | G_A | | | | | | | | | |
| 6 | 6_151949806_ | 151949806 | 151628671 | rs6913578 | C | A | 0.3083 | 0.0393 | 1.0401 | 1.0249 |
| | A_C | | | | | | | | | |
| 6 | 6_151950723_ | 151950723 | 151629588 | rs7774781 | T | C | 0.4098 | −0.0059 | 0.9941 | 0.9952 |
| | C_T | | | | | | | | | |
| 6 | 6_151952332_ | 151952332 | 151631197 | rs9397437 | A | G | 0.0688 | 0.0367 | 1.0374 | 1.0052 |
| | G_A | | | | | | | | | |
| 6 | 6_151955914_ | 151955914 | 151634779 | rs60954078 | G | A | 0.0713 | 0.0741 | 1.0769 | 1.0110 |
| | A_G | | | | | | | | | |
| 6 | 6_151965491_ | 151965491 | 151644356 | rs12197252 | A | G | 0.0929 | 0.0000 | 1.0000 | 1.0000 |
| | G_A | | | | | | | | | |
| 6 | 6_151970639_ | 151970639 | 151649504 | rs1293960 | G | C | 0.5758 | 0.0000 | 1.0000 | 1.0000 |
| | C_G | | | | | | | | | |
| 6 | 6_151985550_ | 151985550 | 151664415 | rs851997 | A | G | 0.3937 | 0.0045 | 1.0045 | 1.0036 |
| | G_A | | | | | | | | | |
| 6 | 6_151985574_ | 151985574 | 151664439 | rs78517863 | C | T | 0.0824 | 0.0000 | 1.0000 | 1.0000 |
| | T_C | | | | | | | | | |
| 6 | 6_152009427_ | 152009427 | 151688292 | rs79347432 | A | G | 0.0269 | −0.0030 | 0.9970 | 0.9998 |
| | G_A | | | | | | | | | |
| 6 | 6_152023191_ | 152023191 | 151702056 | rs851984 | A | G | 0.3965 | 0.0457 | 1.0468 | 1.0374 |
| | G_A | | | | | | | | | |
| 6 | 6_152055978_ | 152055978 | 151734843 | rs6904031 | T | A | 0.0627 | 0.0460 | 1.0471 | 1.0059 |
| | A_T | | | | | | | | | |
| 6 | 6_152117128_ | 152117128 | 151795993 | 6:152117128_ | C | CA | 0.4525 | −0.0086 | 0.9914 | 0.9923 |
| | CA_C | | | CA_C | | | | | | |
| 6 | 6_152156935_ | 152156935 | 151835800 | | A | ATT | 0.1904 | 0.0040 | 1.0040 | 1.0015 |
| | ATT_A | | | | | | | | | |
| 6 | 6_152163381_ | 152163381 | 151842246 | rs9340799 | G | A | 0.3507 | 0.0000 | 1.0000 | 1.0000 |
| | A_G | | | | | | | | | |
| 6 | 6_152263860_ | 152263860 | 151942725 | rs9340898 | T | A | 0.0239 | 0.0000 | 1.0000 | 1.0000 |
| | A_T | | | | | | | | | |
| 6 | 6_152312425_ | 152312425 | 151991290 | rs79784637 | G | T | 0.0206 | −0.0104 | 0.9897 | 0.9996 |
| | T_G | | | | | | | | | |
| 6 | 6_152410883_ | 152410883 | 152089748 | rs1983664 | G | T | 0.0605 | −0.0112 | 0.9889 | 0.9987 |
| | T_G | | | | | | | | | |
| 6 | 6_152428094_ | 152428094 | 152106959 | rs538095870 | CA | C | 0.2291 | 0.0000 | 1.0000 | 1.0000 |
| | C_CA | | | | | | | | | |
| 6 | 6_152428704_ | 152428704 | 152107569 | rs1543403 | G | C | 0.4694 | 0.0000 | 1.0000 | 1.0000 |
| | C_G | | | | | | | | | |
| 6 | 6_152432902_ | 152432902 | 152111767 | rs910416 | T | C | 0.5146 | 0.0428 | 1.0437 | 1.0455 |
| | C_T | | | | | | | | | |
| 6 | 6_152452759_ | 152452759 | 152131624 | rs6925149 | C | T | 0.0609 | 0.0118 | 1.0119 | 1.0014 |
| | T_C | | | | | | | | | |
| 6 | 6_152465616_ | 152465616 | 152144481 | | A | AGT | 0.4279 | 0.0000 | 1.0000 | 1.0000 |
| | AGT_A | | | | | | | | | |
| 6 | 6_152474790_ | 152474790 | 152153655 | rs9397084 | C | T | 0.2141 | 0.0055 | 1.0055 | 1.0024 |
| | T_C | | | | | | | | | |
| 6 | 6_152476784_ | 152476784 | 152155649 | rs6557199 | C | T | 0.0354 | 0.0055 | 1.0055 | 1.0004 |
| | T_C | | | | | | | | | |
| 6 | 6_153099296_ | 153099296 | 152778161 | rs137905783 | C | G | 0.0192 | −0.0206 | 0.9796 | 0.9992 |
| | G_C | | | | | | | | | |
| 6 | 6_155435105_ | 155435105 | 155113971 | rs9371861 | C | A | 0.2313 | −0.0004 | 0.9996 | 0.9998 |
| | A_C | | | | | | | | | |
| 6 | 6_157228314_ | 157228314 | 156907180 | rs28648619 | G | A | 0.0827 | 0.0125 | 1.0126 | 1.0021 |
| | A_G | | | | | | | | | |
| 6 | 6_157331090_ | 157331090 | 157009956 | rs140842923 | T | C | 0.0224 | 0.0176 | 1.0178 | 1.0008 |
| | C_T | | | | | | | | | |
| 6 | 6_159498130_ | 159498130 | 159077098 | rs524410 | T | C | 0.4857 | −0.0025 | 0.9975 | 0.9976 |
| | C_T | | | | | | | | | |
| 6 | 6_159516474_ | 159516474 | 159095442 | rs577038023 | CT | C | 0.1589 | 0.0155 | 1.0156 | 1.0050 |
| | C_CT | | | | | | | | | |
| 6 | 6_161008833_ | 161008833 | 160587801 | rs112021077 | TTCT | T | 0.3315 | 0.0042 | 1.0042 | 1.0028 |
| | T_TTCTG | | | | G | | | | | |
| 6 | 6_161087869_ | 161087869 | 160666837 | rs783148 | T | A | 0.8573 | −0.0047 | 0.9953 | 0.9920 |
| | A_T | | | | | | | | | |
| 6 | 6_163625614_ | 163625614 | 163204582 | rs763599 | T | G | 0.2959 | 0.0048 | 1.0048 | 1.0028 |
| | G_T | | | | | | | | | |
| 6 | 6_164143918_ | 164143918 | 163722886 | rs7766259 | A | G | 0.8970 | 0.0065 | 1.0065 | 1.0117 |
| | G_A | | | | | | | | | |
| 6 | 6_165601875_ | 165601875 | 165188386 | rs116415103 | C | T | 0.0205 | −0.0017 | 0.9983 | 0.9999 |
| | T_C | | | | | | | | | |
| 6 | 6_165610793_ | 165610793 | 165197304 | rs1764051 | G | A | 0.0701 | −0.0014 | 0.9986 | 0.9998 |
| | A_G | | | | | | | | | |
| 6 | 6_166466348_ | 166466348 | 166052860 | rs777580 | A | G | 0.6917 | 0.0007 | 1.0007 | 1.0010 |
| | G_A | | | | | | | | | |
| 6 | 6_166497992_ | 166497992 | 166084504 | 6:166497992_ | T | TAAT | 0.4871 | 0.0019 | 1.0019 | 1.0019 |
| | TAATC_T | | | TAATC_T | | C | | | | |
| 6 | 6_166501349_ | 166501349 | 166087861 | rs113289039 | AG | A | 0.3090 | −0.0112 | 0.9889 | 0.9931 |
| | A_AG | | | | | | | | | |
| 6 | 6_166520692_ | 166520692 | 166107204 | rs2323086 | A | G | 0.5152 | 0.0011 | 1.0011 | 1.0011 |
| | G_A | | | | | | | | | |
| 6 | 6_168292084_ | 168292084 | 167891404 | 6:168292084_ | G | GGTG | 0.9213 | −0.0049 | 0.9951 | 0.9910 |
| | GGTGGGTGT_G | | | GGTGGGTGT_ | | GGTG | | | | |
| | | | | G | | T | | | | |
| 6 | 6_168336052_ | 168336052 | 167935372 | rs3800531 | A | G | 0.3596 | −0.0033 | 0.9967 | 0.9976 |
| | G_A | | | | | | | | | |
| 6 | 6_168378848_ | 168378848 | 167978168 | rs227249 | G | T | 0.5552 | −0.0051 | 0.9949 | 0.9944 |
| | T_G | | | | | | | | | |
| 6 | 6_169006947_ | 169006947 | 168606267 | rs9364472 | G | C | 0.5202 | 0.0000 | 1.0000 | 1.0000 |
| | C_G | | | | | | | | | |
| 6 | 6_169013233_ | 169013233 | 168612553 | rs4464761 | A | G | 0.6596 | −0.0107 | 0.9894 | 0.9860 |
| | G_A | | | | | | | | | |
| 6 | 6_169034960_ | 169034960 | 168634280 | rs2342647 | G | T | 0.3987 | −0.0005 | 0.9995 | 0.9996 |
| | T_G | | | | | | | | | |
| 6 | 6_169072735_ | 169072735 | 168672196 | rs58699559 | T | C | 0.0597 | −0.0028 | 0.9972 | 0.9997 |
| | C_T | | | | | | | | | |
| 6 | 6_169091726_ | 169091726 | 168691612 | rs11966386 | C | A | 0.0120 | −0.0054 | 0.9946 | 0.9999 |
| | A_C | | | | | | | | | |
| 6 | 6_169174555_ | 169174555 | 168774460 | 6:169174555_ | C | CT | 0.8871 | −0.0115 | 0.9886 | 0.9798 |
| | CT_C | | | CT_C | | | | | | |
| 6 | 6_169186803_ | 169186803 | 168786708 | 6:169186803_ | A | ATG | 0.8213 | −0.0009 | 0.9991 | 0.9985 |
| | ATG_A | | | ATG_A | | | | | | |
| 6 | 6_169616697_ | 169616697 | 169216602 | 6:169616697_ | A | AT | 0.5647 | −0.0072 | 0.9928 | 0.9919 |
| | AT_A | | | AT_A | | | | | | |
| 6 | 6_169630340_ | 169630340 | 169230245 | rs6605521 | A | G | 0.5731 | −0.0041 | 0.9959 | 0.9953 |
| | G_A | | | | | | | | | |
| 6 | 6_170332621_ | 170332621 | 170017397 | rs6940159 | C | T | 0.6158 | 0.0190 | 1.0192 | 1.0238 |
| | T_C | | | | | | | | | |
| 6 | 6_170339055_ | 170339055 | 170023831 | rs80097054 | A | G | 0.1077 | 0.0000 | 1.0000 | 1.0000 |
| | G_A | | | | | | | | | |
| 6 | 6_170355128_ | 170355128 | 170039904 | rs114138513 | A | G | 0.0453 | −0.0191 | 0.9811 | 0.9983 |
| | G_A | | | | | | | | | |
| 6 | 6_170789173_ | 170789173 | 170480085 | rs57586097 | T | C | 0.0236 | 0.0339 | 1.0345 | 1.0016 |
| | C_T | | | | | | | | | |
| 7 | 7_1512823_ | 1512823 | 1473187 | rs111256568 | A | G | 0.0293 | −0.0292 | 0.9712 | 0.9983 |
| | G_A | | | | | | | | | |
| 7 | 7_2768355_ | 2768355 | 2728721 | rs4208 | ATG | A | 0.2030 | −0.0040 | 0.9960 | 0.9984 |
| | A_ATG | | | | | | | | | |
| 7 | 7_3563804_ | 3563804 | 3524172 | rs73035954 | T | C | 0.2558 | −0.0019 | 0.9981 | 0.9990 |
| | C_T | | | | | | | | | |
| 7 | 7_3576538_ | 3576538 | 3536906 | rs10234480 | A | G | 0.6309 | −0.0015 | 0.9985 | 0.9981 |
| | G_A | | | | | | | | | |
| 7 | 7_5240541_ | 5240541 | 5200910 | rs4591933 | C | T | 0.6613 | −0.0073 | 0.9927 | 0.9904 |
| | T_C | | | | | | | | | |
| 7 | 7_8820426_ | 8820426 | 8780796 | rs77450427 | A | G | 0.0747 | 0.0032 | 1.0032 | 1.0005 |
| | G_A | | | | | | | | | |
| 7 | 7_10620231_ | 10620231 | 10580604 | rs1981581 | T | G | 0.7575 | 0.0022 | 1.0022 | 1.0033 |
| | G_T | | | | | | | | | |
| 7 | 7_10629885_ | 10629885 | 10590258 | rs10243948 | C | T | 0.5618 | 0.0077 | 1.0077 | 1.0087 |
| | T_C | | | | | | | | | |
| 7 | 7_12592846_ | 12592846 | 12553220 | rs117012007 | G | A | 0.0489 | 0.0227 | 1.0230 | 1.0022 |
| | A_G | | | | | | | | | |
| 7 | 7_12629758_ | 12629758 | 12590133 | rs1138910 | T | G | 0.0952 | 0.0018 | 1.0018 | 1.0003 |
| | G_T | | | | | | | | | |
| 7 | 7_12763696_ | 12763696 | 12724071 | rs10276444 | A | T | 0.2866 | 0.0101 | 1.0102 | 1.0058 |
| | T_A | | | | | | | | | |
| 7 | 7_12771041_ | 12771041 | 12731416 | rs10680080 | TTCT | T | 0.3649 | 0.0006 | 1.0006 | 1.0004 |
| | T_TTCTC | | | | C | | | | | |
| 7 | 7_13884782_ | 13884782 | 13845157 | rs4719382 | G | C | 0.3517 | 0.0042 | 1.0042 | 1.0030 |
| | C_G | | | | | | | | | |
| 7 | 7_16297381_ | 16297381 | 16257756 | rs11769530 | G | A | 0.2080 | −0.0125 | 0.9876 | 0.9948 |
| | A_G | | | | | | | | | |
| 7 | 7_17007625_ | 17007625 | 16968001 | rs1721016 | T | A | 0.4676 | −0.0018 | 0.9982 | 0.9983 |
| | A_T | | | | | | | | | |
| 7 | 7_17057725_ | 17057725 | 17018101 | rs13229759 | G | C | 0.3848 | 0.0000 | 1.0000 | 1.0000 |
| | C_G | | | | | | | | | |
| 7 | 7_17066979_ | 17066979 | 17027355 | rs149535798 | C | T | 0.0202 | −0.0181 | 0.9821 | 0.9993 |
| | T_C | | | | | | | | | |
| 7 | 7_17074440_ | 17074440 | 17034816 | rs471475 | A | G | 0.3963 | 0.0115 | 1.0116 | 1.0092 |
| | G_A | | | | | | | | | |
| 7 | 7_17208250_ | 17208250 | 17168626 | rs75337980 | A | G | 0.0133 | 0.0358 | 1.0364 | 1.0010 |
| | G_A | | | | | | | | | |
| 7 | 7_20508287_ | 20508287 | 20468664 | 7:20508287_C | C | CT | 0.2998 | −0.0076 | 0.9924 | 0.9955 |
| | CT_C | | | T_C | | | | | | |
| 7 | 7_20865979_ | 20865979 | 20826360 | rs12700239 | A | C | 0.1952 | 0.0080 | 1.0080 | 1.0031 |
| | C_A | | | | | | | | | |
| 7 | 7_20885095_ | 20885095 | 20845476 | rs13245023 | G | C | 0.0366 | 0.0054 | 1.0054 | 1.0004 |
| | C_G | | | | | | | | | |
| 7 | 7_20885792_ | 20885792 | 20846173 | rs6965314 | G | A | 0.8525 | −0.0054 | 0.9946 | 0.9908 |
| | A_G | | | | | | | | | |
| 7 | 7_20898650_ | 20898650 | 20859031 | rs113848802 | T | G | 0.0211 | −0.0105 | 0.9896 | 0.9996 |
| | G_T | | | | | | | | | |
| 7 | 7_21168014_ | 21168014 | 21128395 | rs17143912 | C | T | 0.0775 | 0.0125 | 1.0126 | 1.0020 |
| | T_C | | | | | | | | | |
| 7 | 7_21940960_ | 21940960 | 21901342 | rs7971 | G | A | 0.3515 | −0.0236 | 0.9767 | 0.9837 |
| | A_G | | | | | | | | | |
| 7 | 7_21941669_ | 21941669 | 21902051 | rs1128226 | C | A | 0.3746 | −0.0002 | 0.9998 | 0.9999 |
| | A_C | | | | | | | | | |
| 7 | 7_22014189_ | 22014189 | 21974571 | rs756053372 | C | CAA | 0.3789 | 0.0000 | 1.0000 | 1.0000 |
| | CAA_C | | | | | | | | | |
| 7 | 7_22822662_ | 22822662 | 22783043 | rs12530998 | T | A | 0.4042 | −0.0052 | 0.9948 | 0.9958 |
| | A_T | | | | | | | | | |
| 7 | 7_22910541_ | 22910541 | 22870922 | rs117841439 | T | C | 0.1148 | −0.0036 | 0.9964 | 0.9992 |
| | C_T | | | | | | | | | |
| 7 | 7_23488174_ | 23488174 | 23448555 | rs12539834 | C | A | 0.0508 | 0.0045 | 1.0045 | 1.0005 |
| | A_C | | | | | | | | | |
| 7 | 7_23523662_ | 23523662 | 23484043 | | AAAT | AAAT | 0.2545 | 0.0055 | 1.0055 | 1.0028 |
| | AAATAAT_AAAT | | | | | AAT | | | | |
| 7 | 7_23584522_ | 23584522 | 23544903 | rs11767313 | A | C | 0.1813 | 0.0000 | 1.0000 | 1.0000 |
| | C_A | | | | | | | | | |
| 7 | 7_25163744_ | 25163744 | 25124125 | rs11267038 | C | A | 0.0680 | −0.0105 | 0.9896 | 0.9986 |
| | A_C | | | | | | | | | |
| 7 | 7_25569548_ | 25569548 | 25529928 | rs289997 | T | C | 0.1667 | −0.0063 | 0.9937 | 0.9979 |
| | C_T | | | | | | | | | |
| 7 | 7_25573204_ | 25573204 | 25533584 | rs965610 | A | G | 0.2347 | −0.0070 | 0.9930 | 0.9967 |
| | G_A | | | | | | | | | |
| 7 | 7_25591710_ | 25591710 | 25552090 | rs73277029 | T | C | 0.1744 | −0.0067 | 0.9933 | 0.9977 |
| | C_T | | | | | | | | | |
| 7 | 7_25656363_ | 25656363 | 25616743 | rs73085996 | C | G | 0.2302 | 0.0052 | 1.0052 | 1.0024 |
| | G_C | | | | | | | | | |
| 7 | 7_27509178_ | 27509178 | 27469559 | rs73077497 | G | A | 0.0379 | 0.0262 | 1.0265 | 1.0020 |
| | A_G | | | | | | | | | |
| 7 | 7_27617028_ | 27617028 | 27577409 | rs140387951 | A | G | 0.0410 | 0.0119 | 1.0120 | 1.0010 |
| | G_A | | | | | | | | | |
| 7 | 7_28128002_ | 28128002 | 28088383 | rs7457754 | C | T | 0.1467 | 0.0039 | 1.0039 | 1.0011 |
| | T_C | | | | | | | | | |
| 7 | 7_28209953_ | 28209953 | 28170334 | 7:28209953_G | G | GAT | 0.5268 | 0.0074 | 1.0074 | 1.0078 |
| | GAT_G | | | AT_G | | | | | | |
| 7 | 7_28356889_ | 28356889 | 28317270 | rs17156577 | C | T | 0.1139 | 0.0105 | 1.0106 | 1.0024 |
| | T_C | | | | | | | | | |
| 7 | 7_28627353_ | 28627353 | 28587735 | rs141085164 | T | C | 0.0109 | −0.0195 | 0.9807 | 0.9996 |
| | C_T | | | | | | | | | |
| 7 | 7_28652801_ | 28652801 | 28613184 | rs189567370 | G | A | 0.0123 | −0.0105 | 0.9896 | 0.9997 |
| | A_G | | | | | | | | | |
| 7 | 7_28856014_ | 28856014 | 28816397 | rs10486594 | C | T | 0.1048 | −0.0170 | 0.9831 | 0.9965 |
| | T_C | | | | | | | | | |
| 7 | 7_28938590_ | 28938590 | 28898973 | rs10250382 | C | G | 0.5041 | 0.0073 | 1.0073 | 1.0074 |
| | G_C | | | | | | | | | |
| 7 | 7_29121211_ | 29121211 | 29081595 | rs317726 | C | T | 0.5171 | 0.0025 | 1.0025 | 1.0026 |
| | T_C | | | | | | | | | |
| 7 | 7_29468698_ | 29468698 | 29429082 | rs7785829 | A | G | 0.6103 | 0.0141 | 1.0142 | 1.0174 |
| | G_A | | | | | | | | | |
| 7 | 7_29752475_ | 29752475 | 29712859 | rs62458273 | G | A | 0.1310 | −0.0045 | 0.9955 | 0.9988 |
| | A_G | | | | | | | | | |
| 7 | 7_31893072_ | 31893072 | 31853458 | rs76907223 | G | C | 0.0619 | 0.0109 | 1.0110 | 1.0014 |
| | C_G | | | | | | | | | |
| 7 | 7_33484507_ | 33484507 | 33444895 | rs1860601 | G | T | 0.2449 | 0.0031 | 1.0031 | 1.0015 |
| | T_G | | | | | | | | | |
| 7 | 7_36706015_ | 36706015 | 36666410 | rs117705242 | G | A | 0.0231 | −0.0139 | 0.9862 | 0.9994 |
| | A_G | | | | | | | | | |
| 7 | 7_43291856_ | 43291856 | 43252257 | rs57648935 | C | T | 0.3892 | −0.0067 | 0.9933 | 0.9948 |
| | T_C | | | | | | | | | |
| 7 | 7_43299616_ | 43299616 | 43260017 | rs9639872 | C | T | 0.3459 | 0.0000 | 1.0000 | 1.0000 |
| | T_C | | | | | | | | | |
| 7 | 7_43889991_ | 43889991 | 43850392 | rs4724250 | A | C | 0.1457 | 0.0004 | 1.0004 | 1.0001 |
| | C_A | | | | | | | | | |
| 7 | 7_44790921_ | 44790921 | 44751322 | rs55705735 | C | T | 0.1252 | 0.0088 | 1.0088 | 1.0022 |
| | T_C | | | | | | | | | |
| 7 | 7_44961337_ | 44961337 | 44921738 | rs55738054 | A | G | 0.0639 | 0.0194 | 1.0196 | 1.0025 |
| | G_A | | | | | | | | | |
| 7 | 7_44961407_ | 44961407 | 44921808 | rs73102191 | G | T | 0.0148 | 0.0019 | 1.0019 | 1.0001 |
| | T_G | | | | | | | | | |
| 7 | 7_47022767_ | 47022767 | 46983169 | rs6955374 | A | G | 0.3530 | 0.0053 | 1.0053 | 1.0038 |
| | G_A | | | | | | | | | |
| 7 | 7_47100078_ | 47100078 | 47060480 | rs56017904 | T | C | 0.1214 | −0.0127 | 0.9874 | 0.9969 |
| | C_T | | | | | | | | | |
| 7 | 7_47673601_ | 47673601 | 47634003 | rs73697710 | C | T | 0.0108 | 0.0203 | 1.0205 | 1.0004 |
| | T_C | | | | | | | | | |
| 7 | 7_47820962_ | 47820962 | 47781364 | rs6953528 | C | T | 0.3646 | −0.0063 | 0.9937 | 0.9954 |
| | T_C | | | | | | | | | |
| 7 | 7_47831912_ | 47831912 | 47792314 | rs10282416 | T | G | 0.8159 | −0.0018 | 0.9982 | 0.9971 |
| | G_T | | | | | | | | | |
| 7 | 7_47875564_ | 47875564 | 47835966 | rs4543454 | T | C | 0.4036 | −0.0064 | 0.9936 | 0.9949 |
| | C_T | | | | | | | | | |
| 7 | 7_48472383_ | 48472383 | 48432786 | rs7801736 | A | G | 0.5402 | −0.0027 | 0.9973 | 0.9971 |
| | G_A | | | | | | | | | |
| 7 | 7_50764404_ | 50764404 | 50696707 | rs2299156 | T | C | 0.2009 | 0.0146 | 1.0147 | 1.0059 |
| | C_T | | | | | | | | | |
| 7 | 7_50923571_ | 50923571 | 50855874 | rs17454681 | C | T | 0.5260 | 0.0056 | 1.0056 | 1.0059 |
| | T_C | | | | | | | | | |
| 7 | 7_51166631_ | 51166631 | 51098934 | rs4948187 | G | A | 0.3921 | −0.0014 | 0.9986 | 0.9989 |
| | A_G | | | | | | | | | |
| 7 | 7_55192256_ | 55192256 | 55124563 | rs13244925 | C | A | 0.5497 | −0.0081 | 0.9919 | 0.9912 |
| | A_C | | | | | | | | | |
| 7 | 7_57401795_ | 57401795 | 57334088 | rs11767308 | T | C | 0.0551 | 0.0000 | 1.0000 | 1.0000 |
| | C_T | | | | | | | | | |
| 7 | 7_63276830_ | 63276830 | 63816452 | rs1708651 | C | T | 0.6040 | 0.0066 | 1.0066 | 1.0080 |
| | T_C | | | | | | | | | |
| 7 | 7_67991296_ | 67991296 | 68526309 | 7:67991296_G | G | GAGG | 0.3327 | −0.0014 | 0.9986 | 0.9991 |
| | GAGGAAGGA_G | | | AGGAAGGA_G | | AAGG | | | | |
| | | | | | | A | | | | |
| 7 | 7_71603177_ | 71603177 | 72138192 | rs6970190 | C | T | 0.0210 | 0.0252 | 1.0255 | 1.0011 |
| | T_C | | | | | | | | | |
| 7 | 7_73056750_ | 73056750 | 73642420 | rs6957745 | C | T | 0.1898 | 0.0076 | 1.0076 | 1.0029 |
| | T_C | | | | | | | | | |
| 7 | 7_74110705_ | 74110705 | 74696373 | rs74910854 | G | A | 0.0700 | 0.0102 | 1.0103 | 1.0014 |
| | A_G | | | | | | | | | |
| 7 | 7_74231353_ | 74231353 | 74816826 | rs707402 | G | A | 0.7197 | 0.0000 | 1.0000 | 1.0000 |
| | A_G | | | | | | | | | |
| 7 | 7_74279763_ | 74279763 | 74863463 | rs801066 | T | C | 0.1925 | 0.0031 | 1.0031 | 1.0012 |
| | C_T | | | | | | | | | |
| 7 | 7_74350606_ | 74350606 | 74935609 | rs199562542 | CA | C | 0.2773 | 0.0000 | 1.0000 | 1.0000 |
| | C_CA | | | | | | | | | |
| 7 | 7_74359358_ | 74359358 | 74944369 | rs1637365 | T | C | 0.2792 | 0.0074 | 1.0074 | 1.0042 |
| | C_T | | | | | | | | | |
| 7 | 7_75168562_ | 75168562 | 75539242 | rs1167835 | A | G | 0.2281 | 0.0025 | 1.0025 | 1.0011 |
| | G_A | | | | | | | | | |
| 7 | 7_77062679_ | 77062679 | 77433362 | rs998994 | C | A | 0.4996 | 0.0075 | 1.0075 | 1.0075 |
| | A_C | | | | | | | | | |
| 7 | 7_77954163_ | 77954163 | 78324846 | rs798301 | A | T | 0.4807 | 0.0000 | 1.0000 | 1.0000 |
| | T_A | | | | | | | | | |
| 7 | 7_77992996_ | 77992996 | 78363679 | rs4476926 | G | A | 0.1428 | −0.0139 | 0.9862 | 0.9961 |
| | A_G | | | | | | | | | |
| 7 | 7_78463911_ | 78463911 | 78834595 | rs8180745 | G | A | 0.0463 | −0.0162 | 0.9839 | 0.9985 |
| | A_G | | | | | | | | | |
| 7 | 7_82079457_ | 82079457 | 82450141 | rs117603127 | A | G | 0.0139 | −0.0582 | 0.9435 | 0.9984 |
| | G_A | | | | | | | | | |
| 7 | 7_86345495_ | 86345495 | 86716179 | rs2214653 | A | G | 0.4112 | 0.0003 | 1.0003 | 1.0002 |
| | G_A | | | | | | | | | |
| 7 | 7_86348037_ | 86348037 | 86718721 | rs2237547 | G | A | 0.2932 | −0.0039 | 0.9961 | 0.9977 |
| | A_G | | | | | | | | | |
| 7 | 7_86372202_ | 86372202 | 86742886 | rs12704285 | A | G | 0.0219 | −0.0223 | 0.9779 | 0.9990 |
| | G_A | | | | | | | | | |
| 7 | 7_86474313_ | 86474313 | 86844997 | rs1989796 | C | T | 0.5923 | −0.0108 | 0.9893 | 0.9873 |
| | T_C | | | | | | | | | |
| 7 | 7_86662659_ | 86662659 | 87033343 | rs6953148 | C | A | 0.2920 | −0.0010 | 0.9990 | 0.9994 |
| | A_C | | | | | | | | | |
| 7 | 7_88411073_ | 88411073 | 88781759 | rs3847036 | G | A | 0.9224 | −0.0038 | 0.9962 | 0.9930 |
| | A_G | | | | | | | | | |
| 7 | 7_89529281_ | 89529281 | 89899967 | rs148300099 | C | T | 0.6557 | −0.0106 | 0.9895 | 0.9862 |
| | T_C | | | | | | | | | |
| 7 | 7_89533047_ | 89533047 | 89903733 | rs11489497 | G | C | 0.3065 | −0.0029 | 0.9971 | 0.9982 |
| | C_G | | | | | | | | | |
| 7 | 7_90086677_ | 90086677 | 90457363 | rs139526283 | GA | G | 0.1507 | 0.0116 | 1.0117 | 1.0035 |
| | G_GA | | | | | | | | | |
| 7 | 7_91459189_A_AT | 91459189 | 91829875 | | ATT | A | 0.3286 | 0.0151 | 1.0152 | 1.0100 |
| | T | | | | | | | | | |
| 7 | 7_91565914_T_TA | 91565914 | 91936600 | rs35469924 | TA | T | 0.5413 | −0.0085 | 0.9915 | 0.9909 |
| 7 | 7_94090342_C_T | 94090342 | 94461030 | rs78582026 | T | C | 0.1436 | 0.0043 | 1.0043 | 1.0012 |
| 7 | 7_94113799_T_C | 94113799 | 94484487 | rs17268829 | C | T | 0.2792 | 0.0112 | 1.0113 | 1.0063 |
| 7 | 7_94321785_A_AT | 94321785 | 94692473 | | ATT | A | 0.3141 | 0.0023 | 1.0023 | 1.0014 |
| | T | | | | | | | | | |
| 7 | 7_94420291_G_A | 94420291 | 94790979 | rs35665648 | A | G | 0.1820 | −0.0055 | 0.9945 | 0.9980 |
| 7 | 7_94609593_G_A | 94609593 | 94980281 | rs149550563 | A | G | 0.0106 | 0.0278 | 1.0282 | 1.0006 |
| 7 | 7_94979963_C_T | 94979963 | 95350651 | rs17880824 | T | C | 0.0145 | 0.0000 | 1.0000 | 1.0000 |
| 7 | 7_97686264_A_G | 97686264 | 98056952 | rs12704958 | G | A | 0.0591 | 0.0230 | 1.0233 | 1.0028 |
| 7 | 7_97865802_C_A | 97865802 | 98236490 | rs118053816 | A | C | 0.0205 | −0.0165 | 0.9836 | 0.9993 |
| 7 | 7_98005235_G_A | 98005235 | 98375923 | rs4439053 | A | G | 0.1627 | −0.0150 | 0.9851 | 0.9952 |
| 7 | 7_99292372_A_G | 99292372 | 99694749 | rs112779675 | G | A | 0.0179 | 0.0009 | 1.0009 | 1.0000 |
| 7 | 7_99576068_G_A | 99576068 | 99978445 | rs12673005 | A | G | 0.3884 | 0.0010 | 1.0010 | 1.0008 |
| 7 | 7_99893765_C_T | 99893765 | 100296142 | rs73161745 | T | C | 0.2963 | 0.0016 | 1.0016 | 1.0009 |
| 7 | 7_99948655_T_G | 99948655 | 100351032 | rs111963714 | G | T | 0.2109 | 0.0116 | 1.0117 | 1.0049 |
| 7 | 7_100008270_T_C | 100008270 | 100410647 | rs192787100 | C | T | 0.0116 | 0.0240 | 1.0243 | 1.0006 |
| 7 | 7_100035274_G_A | 100035274 | 100437651 | rs117911427 | A | G | 0.0215 | 0.0085 | 1.0085 | 1.0004 |
| 7 | 7_100532911_C_T | 100532911 | 100935285 | rs6465777 | T | C | 0.2644 | −0.0034 | 0.9966 | 0.9982 |
| 7 | 7_100781615_G_A | 100781615 | 101138334 | rs1050813 | A | G | 0.2012 | 0.0078 | 1.0078 | 1.0032 |
| 7 | 7_101437919_TA_T | 101437919 | 101794639 | 7:101437919_ | T | TA | 0.3632 | −0.0003 | 0.9997 | 0.9998 |
| | | | | TA_T | | | | | | |
| 7 | 7_101552440_G_A | 101552440 | 101909160 | rs71559437 | A | G | 0.1255 | −0.0051 | 0.9949 | 0.9987 |
| 7 | 7_101556363_C_T | 101556363 | 101913083 | rs11974778 | T | C | 0.2798 | −0.0108 | 0.9893 | 0.9940 |
| 7 | 7_101561492_C_T | 101561492 | 101918212 | rs73185821 | T | C | 0.0701 | −0.0174 | 0.9828 | 0.9976 |
| 7 | 7_101591393_G_A | 101591393 | 101948113 | rs80173192 | A | G | 0.0112 | −0.0086 | 0.9914 | 0.9998 |
| 7 | 7_101842782_A_G | 101842782 | 102199502 | rs1297632 | G | A | 0.9537 | 0.0055 | 1.0055 | 1.0105 |
| 7 | 7_102085389_T_C | 102085389 | 102444942 | rs11983129 | C | T | 0.0725 | 0.0139 | 1.0140 | 1.0020 |
| 7 | 7_102088402_A_G | 102088402 | 102447955 | rs6465864 | G | A | 0.9090 | −0.0094 | 0.9906 | 0.9831 |
| 7 | 7_102481842_T_C | 102481842 | 102841395 | rs7800548 | C | T | 0.3416 | 0.0095 | 1.0095 | 1.0065 |
| 7 | 7_102607417_G_T | 102607417 | 102966970 | rs140632113 | T | G | 0.0414 | 0.0137 | 1.0138 | 1.0011 |
| 7 | 7_102891784_T_C | 102891784 | 103251337 | rs6946892 | C | T | 0.7472 | −0.0083 | 0.9917 | 0.9877 |
| 7 | 7_103057977_CT_C | 103057977 | 103417530 | 7:103057977_ | C | CT | 0.0592 | 0.0078 | 1.0078 | 1.0009 |
| | | | | CT_C | | | | | | |
| 7 | 7_103451091_A_G | 103451091 | 103810644 | rs39332 | G | A | 0.2259 | 0.0051 | 1.0051 | 1.0023 |
| 7 | 7_106148554_C_A | 106148554 | 106508108 | rs75292889 | A | C | 0.0843 | 0.0015 | 1.0015 | 1.0003 |
| 7 | 7_106715221_C_T | 106715221 | 107074776 | rs12669339 | T | C | 0.4433 | 0.0044 | 1.0044 | 1.0039 |
| 7 | 7_107160253_C_T | 107160253 | 107519808 | rs2520253 | T | C | 0.4695 | −0.0088 | 0.9912 | 0.9918 |
| 7 | 7_109304110_C_A | 109304110 | 109664053 | rs3915078 | A | C | 0.2788 | −0.0019 | 0.9981 | 0.9989 |
| 7 | 7_109326883_G_G | 109326883 | 109686826 | rs572979797 | GA | G | 0.3436 | −0.0012 | 0.9988 | 0.9992 |
| | A | | | | | | | | | |
| 7 | 7_109373934_A_T | 109373934 | 109733877 | rs79862777 | T | A | 0.3770 | 0.0000 | 1.0000 | 1.0000 |
| 7 | 7_114352862_C_T | 114352862 | 114712807 | rs776472 | T | C | 0.5775 | 0.0072 | 1.0072 | 1.0084 |
| 7 | 7_121950965_A_G | 121950965 | 122310911 | rs7779018 | G | A | 0.4174 | −0.0018 | 0.9982 | 0.9985 |
| 7 | 7_126090509_C_T | 126090509 | 126450455 | rs13247338 | T | C | 0.1403 | −0.0140 | 0.9861 | 0.9961 |
| 7 | 7_129217706_C_T | 129217706 | 129577865 | rs12673568 | T | C | 0.5471 | 0.0000 | 1.0000 | 1.0000 |
| 7 | 7_130432913_A_A | 130432913 | 130748154 | rs553015785 | AT | A | 0.4731 | 0.0102 | 1.0103 | 1.0097 |
| | T | | | | | | | | | |
| 7 | 7_130607688_C_T | 130607688 | 130922929 | rs73161912 | T | C | 0.2992 | −0.0010 | 0.9990 | 0.9994 |
| 7 | 7_130627014_G_T | 130627014 | 130942255 | rs12533596 | T | G | 0.2236 | −0.0067 | 0.9933 | 0.9970 |
| 7 | 7_130656911_C_T | 130656911 | 130972152 | rs12706954 | T | C | 0.3734 | −0.0045 | 0.9955 | 0.9966 |
| 7 | 7_130657509_C_G | 130657509 | 130972750 | rs205733 | G | C | 0.5361 | 0.0087 | 1.0087 | 1.0094 |
| 7 | 7_130666992_C_C | 130666992 | 130982233 | rs5887486 | CA | C | 0.3634 | −0.0019 | 0.9981 | 0.9986 |
| | A | | | | | | | | | |
| 7 | 7_130667258_G_A | 130667258 | 130982499 | rs4141794 | A | G | 0.3684 | −0.0062 | 0.9938 | 0.9955 |
| 7 | 7_130667323_A_C | 130667323 | 130982564 | rs205752 | C | A | 0.5683 | 0.0025 | 1.0025 | 1.0028 |
| 7 | 7_130668383_T_C | 130668383 | 130983624 | rs6973318 | C | T | 0.6341 | 0.0012 | 1.0012 | 1.0015 |
| 7 | 7_130674481_G_A | 130674481 | 130989722 | rs68056147 | A | G | 0.2971 | 0.0101 | 1.0102 | 1.0060 |
| 7 | 7_130688577_G_A | 130688577 | 131003818 | rs983173 | A | G | 0.5633 | −0.0086 | 0.9914 | 0.9904 |
| 7 | 7_130984689_G_A | 130984689 | 131299930 | rs7812294 | A | G | 0.1644 | −0.0029 | 0.9971 | 0.9990 |
| 7 | 7_131009008_G_C | 131009008 | 131324249 | rs10233294 | C | G | 0.0526 | −0.0231 | 0.9772 | 0.9976 |
| 7 | 7_131349841_AT_ | 131349841 | 131665082 | 7:131349841_ | A | AT | 0.6715 | −0.0062 | 0.9938 | 0.9917 |
| | A | | | AT_A | | | | | | |
| 7 | 7_135015546_C_T | 135015546 | 135330794 | rs12532231 | T | C | 0.1995 | 0.0007 | 1.0007 | 1.0003 |
| 7 | 7_135358202_A_G | 135358202 | 135673454 | rs79580568 | G | A | 0.1280 | −0.0181 | 0.9821 | 0.9954 |
| 7 | 7_135843296_C_T | 135843296 | 136158548 | rs55875516 | T | C | 0.2567 | 0.0077 | 1.0077 | 1.0040 |
| 7 | 7_135853583_G_A | 135853583 | 136168835 | rs756421 | A | G | 0.4022 | 0.0028 | 1.0028 | 1.0023 |
| 7 | 7_139835660_C_T | 139835660 | 140135860 | rs79485117 | T | C | 0.0882 | −0.0181 | 0.9821 | 0.9968 |
| 7 | 7_139943702_CT_C | 139943702 | 140243902 | rs201664599 | C | CT | 0.5381 | 0.0167 | 1.0168 | 1.0182 |
| 7 | 7_139943889_A_G | 139943889 | 140244089 | rs2003526 | G | A | 0.5509 | 0.0000 | 1.0000 | 1.0000 |
| 7 | 7_139946053_A_G | 139946053 | 140246253 | rs13225058 | G | A | 0.3330 | 0.0099 | 1.0099 | 1.0066 |
| 7 | 7_139950193_A_A | 139950193 | 140250393 | rs71170962 | AT | A | 0.2221 | 0.0048 | 1.0048 | 1.0021 |
| | T | | | | | | | | | |
| 7 | 7_139954640_C_A | 139954640 | 140254840 | rs12703654 | A | C | 0.4571 | 0.0046 | 1.0046 | 1.0042 |
| 7 | 7_142355935_A_C | 142355935 | 142648438 | rs2226970 | C | A | 0.3990 | −0.0053 | 0.9947 | 0.9958 |
| 7 | 7_144048902_G_T | 144048902 | 144351809 | rs62485509 | T | G | 0.2284 | −0.0219 | 0.9783 | 0.9901 |
| 7 | 7_144074929_G_A | 144074929 | 144377836 | rs720475 | A | G | 0.2520 | 0.0000 | 1.0000 | 1.0000 |
| 7 | 7_144079218_G_A | 144079218 | 144382125 | rs1635083 | A | G | 0.8585 | −0.0019 | 0.9981 | 0.9967 |
| 7 | 7_144149298_A_G | 144149298 | 144452205 | rs117837628 | G | A | 0.0158 | −0.0158 | 0.9843 | 0.9995 |
| 7 | 7_144331267_G_A | 144331267 | 144634174 | rs78749260 | A | G | 0.0206 | 0.0048 | 1.0048 | 1.0002 |
| 7 | 7_144906844_G_A | 144906844 | 145209751 | rs7791708 | A | G | 0.6742 | −0.0066 | 0.9934 | 0.9911 |
| 7 | 7_148739428_A_G | 148739428 | 149042336 | rs10808055 | G | A | 0.9370 | −0.0097 | 0.9903 | 0.9820 |
| 7 | 7_150121933_G_A | 150121933 | 150424845 | rs10268592 | A | G | 0.7453 | −0.0057 | 0.9943 | 0.9915 |
| 7 | 7_150542611_A_A | 150542611 | 150845523 | rs150149364 | AT | A | 0.1198 | −0.0035 | 0.9965 | 0.9992 |
| | T | | | | | | | | | |
| 7 | 7_150542646_G_A | 150542646 | 150845558 | rs6977381 | A | G | 0.5538 | −0.0022 | 0.9978 | 0.9976 |
| 7 | 7_150542711_G_A | 150542711 | 150845623 | rs6977416 | A | G | 0.3542 | −0.0035 | 0.9965 | 0.9975 |
| 7 | 7_151092104_CTG | 151092104 | 151395018 | rs754370996 | C | CTG | 0.6263 | −0.0002 | 0.9998 | 0.9997 |
| | _C | | | | | | | | | |
| 7 | 7_151092578_A_A | 151092578 | 151395492 | rs200990584 | AGG | A | 0.7033 | −0.0015 | 0.9985 | 0.9979 |
| | GG | | | | | | | | | |
| 7 | 7_151538608_G_T | 151538608 | 151841522 | rs62638844 | T | G | 0.7029 | −0.0069 | 0.9931 | 0.9904 |
| 7 | 7_152174826_G_C | 152174826 | 152477741 | rs11765960 | C | G | 0.6782 | 0.0057 | 1.0057 | 1.0078 |
| 7 | 7_152336554_A_A | 152336554 | 152639469 | rs372925374 | AAAA | A | 0.6708 | 0.0041 | 1.0041 | 1.0055 |
| | AAAT | | | | T | | | | | |
| 7 | 7_152676742_TA_T | 152676742 | 152979657 | 7:152676742_ | T | TA | 0.0247 | 0.0226 | 1.0229 | 1.0011 |
| | | | | TA_T | | | | | | |
| 7 | 7_152697402_T_A | 152697402 | 153000317 | rs114108757 | A | T | 0.0454 | 0.0019 | 1.0019 | 1.0002 |
| 7 | 7_154995361_G_A | 154995361 | 155203651 | rs114735553 | A | G | 0.0430 | 0.0235 | 1.0238 | 1.0020 |
| 7 | 7_155527763_T_C | 155527763 | 155735069 | rs61071575 | C | T | 0.0470 | −0.0156 | 0.9845 | 0.9985 |
| 7 | 7_155734665_A_G | 155734665 | 155941971 | rs4716944 | G | A | 0.5497 | −0.0083 | 0.9917 | 0.9909 |
| 7 | 7_155737385_C_T | 155737385 | 155944691 | rs12698353 | T | C | 0.4532 | −0.0007 | 0.9993 | 0.9994 |
| 7 | 7_155740535_T_A | 155740535 | 155947841 | rs11764203 | A | T | 0.4545 | −0.0004 | 0.9996 | 0.9996 |
| 8 | 8_156768_G_A | 156768 | 206768 | rs10086982 | A | G | 0.2619 | 0.0000 | 1.0000 | 1.0000 |
| 8 | 8_163366_T_C | 163366 | 213366 | rs34810249 | C | T | 0.2384 | 0.0065 | 1.0065 | 1.0031 |
| 8 | 8_174546_G_A | 174546 | 224546 | rs2906324 | A | G | 0.2294 | 0.0000 | 1.0000 | 1.0000 |
| 8 | 8_182807_A_G | 182807 | 232807 | rs2286137 | G | A | 0.3447 | 0.0080 | 1.0080 | 1.0055 |
| 8 | 8_185668_C_T | 185668 | 235668 | rs10093989 | T | C | 0.1778 | 0.0000 | 1.0000 | 1.0000 |
| 8 | 8_235700_C_T | 235700 | 285700 | rs6558572 | T | C | 0.3152 | 0.0087 | 1.0087 | 1.0055 |
| 8 | 8_1169041_C_T | 1169041 | 1219041 | rs147988121 | T | C | 0.0123 | −0.0305 | 0.9700 | 0.9993 |
| 8 | 8_4140685_G_T | 4140685 | 4283163 | rs2680602 | T | G | 0.0465 | 0.0020 | 1.0020 | 1.0002 |
| 8 | 8_4261060_A_T | 4261060 | 4403538 | rs13267484 | T | A | 0.2565 | 0.0018 | 1.0018 | 1.0009 |
| 8 | 8_4293754_A_G | 4293754 | 4436232 | rs73178944 | G | A | 0.0793 | 0.0224 | 1.0227 | 1.0036 |
| 8 | 8_5422803_G_T | 5422803 | 5565281 | rs7015839 | T | G | 0.0109 | −0.0466 | 0.9545 | 0.9990 |
| 8 | 8_5645808_C_G | 5645808 | 5788286 | rs7463406 | G | C | 0.7231 | −0.0047 | 0.9953 | 0.9932 |
| 8 | 8_5683118_G_A | 5683118 | 5825596 | rs12678253 | A | G | 0.3175 | 0.0085 | 1.0085 | 1.0054 |
| 8 | 8_5973923_A_G | 5973923 | 6116401 | rs11786309 | G | A | 0.0186 | 0.0321 | 1.0326 | 1.0012 |
| 8 | 8_6743729_G_C | 6743729 | 6886207 | rs10110272 | C | G | 0.0970 | −0.0200 | 0.9802 | 0.9962 |
| 8 | 8_8270294_T_G | 8270294 | 8412784 | rs6601697 | G | T | 0.6394 | −0.0097 | 0.9903 | 0.9877 |
| 8 | 8_8287139_G_A | 8287139 | 8429629 | rs73496865 | A | G | 0.5280 | −0.0016 | 0.9984 | 0.9983 |
| 8 | 8_10445702_G_A | 10445702 | 10588192 | rs12543042 | A | G | 0.1970 | −0.0025 | 0.9975 | 0.9990 |
| 8 | 8_10465942_T_A | 10465942 | 10608432 | rs28446662 | A | T | 0.2215 | −0.0071 | 0.9929 | 0.9969 |
| 8 | 8_11094473_C_T | 11094473 | 11236964 | rs11250124 | T | C | 0.0110 | −0.0206 | 0.9796 | 0.9996 |
| 8 | 8_11368283_CAAA | 11368283 | 11510774 | 8:11368283_C | C | CAAA | 0.5945 | −0.0020 | 0.9980 | 0.9976 |
| | T_C | | | AAAT_C | | T | | | | |
| 8 | 8_11485820_C_T | 11485820 | 11628311 | rs7837995 | T | C | 0.4589 | −0.0068 | 0.9932 | 0.9938 |
| 8 | 8_11495497_T_A | 11495497 | 11637988 | | A | T | 0.4038 | −0.0020 | 0.9980 | 0.9984 |
| 8 | 8_13687830_G_A | 13687830 | 13830321 | rs10283191 | A | G | 0.2909 | 0.0037 | 1.0037 | 1.0022 |
| 8 | 8_15657837_G_C | 15657837 | 15800328 | rs2604384 | C | G | 0.4819 | −0.0042 | 0.9958 | 0.9960 |
| 8 | 8_16080739_T_C | 16080739 | 16223230 | rs7819754 | C | T | 0.7365 | 0.0006 | 1.0006 | 1.0009 |
| 8 | 8_16087631_C_T | 16087631 | 16230122 | rs10101109 | T | C | 0.6993 | 0.0052 | 1.0052 | 1.0073 |
| 8 | 8_16087729_G_A | 16087729 | 16230220 | rs10100384 | A | G | 0.6307 | 0.0008 | 1.0008 | 1.0010 |
| 8 | 8_16125987_T_C | 16125987 | 16268478 | rs1480147 | C | T | 0.3135 | −0.0032 | 0.9968 | 0.9980 |
| 8 | 8_16418358_T_C | 16418358 | 16560849 | rs17123231 | C | T | 0.0562 | 0.0143 | 1.0144 | 1.0016 |
| 8 | 8_16418370_A_G | 16418370 | 16560861 | rs10094727 | G | A | 0.9161 | −0.0021 | 0.9979 | 0.9962 |
| 8 | 8_17051352_C_T | 17051352 | 17193843 | rs36026940 | T | C | 0.0681 | 0.0004 | 1.0004 | 1.0001 |
| 8 | 8_17083095_T_C | 17083095 | 17225586 | rs73198600 | C | T | 0.0525 | −0.0076 | 0.9924 | 0.9992 |
| 8 | 8_17531802_T_G | 17531802 | 17674293 | rs28679501 | G | T | 0.6962 | 0.0042 | 1.0042 | 1.0059 |
| 8 | 8_17787610_CT_C | 17787610 | 17930101 | 8:17787610_C | C | CT | 0.6230 | −0.0091 | 0.9909 | 0.9887 |
| | | | | T_C | | | | | | |
| 8 | 8_17848555_AT_A | 17848555 | 17991046 | | ATT | AT | 0.1317 | 0.0026 | 1.0026 | 1.0007 |
| | TT | | | | | | | | | |
| 8 | 8_17872760_T_TA | 17872760 | 18015251 | | TA | T | 0.6157 | −0.0014 | 0.9986 | 0.9983 |
| 8 | 8_18056532_C_CT | 18056532 | 18199023 | rs35361039 | CT | C | 0.0413 | 0.0153 | 1.0154 | 1.0013 |
| 8 | 8_19685909_G_T | 19685909 | 19828398 | rs7017697 | T | G | 0.6686 | −0.0029 | 0.9971 | 0.9961 |
| 8 | 8_21760026_A_G | 21760026 | 21902515 | rs7017582 | G | A | 0.8838 | 0.0076 | 1.0076 | 1.0135 |
| 8 | 8_21773384_C_T | 21773384 | 21915873 | rs11783414 | T | C | 0.5060 | −0.0026 | 0.9974 | 0.9974 |
| 8 | 8_23024091_A_G | 23024091 | 23166578 | rs11135706 | G | A | 0.6959 | −0.0099 | 0.9901 | 0.9863 |
| 8 | 8_23377604_A_C | 23377604 | 23520091 | rs36104352 | C | A | 0.1114 | −0.0113 | 0.9888 | 0.9975 |
| 8 | 8_23447496_A_G | 23447496 | 23589983 | rs1028016 | G | A | 0.6487 | −0.0038 | 0.9962 | 0.9951 |
| 8 | 8_23465063_C_A | 23465063 | 23607550 | rs13264439 | A | C | 0.4385 | −0.0095 | 0.9905 | 0.9917 |
| 8 | 8_23654713_G_A | 23654713 | 23797200 | rs182563 | A | G | 0.3615 | 0.0000 | 1.0000 | 1.0000 |
| 8 | 8_23663653_C_A | 23663653 | 23806140 | rs310295 | A | C | 0.4032 | 0.0031 | 1.0031 | 1.0025 |
| 8 | 8_23668492_T_TTA | 23668492 | 23810979 | rs141496299 | TTA | T | 0.3131 | 0.0098 | 1.0098 | 1.0062 |
| 8 | 8_25399102_C_T | 25399102 | 25541586 | rs7387502 | T | C | 0.2489 | −0.0160 | 0.9841 | 0.9921 |
| 8 | 8_25831778_C_T | 25831778 | 25974262 | rs13256025 | T | C | 0.2047 | 0.0035 | 1.0035 | 1.0014 |
| 8 | 8_25936499_A_G | 25936499 | 26078983 | rs138748843 | G | A | 0.0224 | 0.0199 | 1.0201 | 1.0009 |
| 8 | 8_27424542_CAAA | 27424542 | 27567025 | rs758758659 | C | CAAA | 0.2934 | −0.0008 | 0.9992 | 0.9995 |
| | AAA_C | | | | | AAA | | | | |
| 8 | 8_27425564_T_TA | 27425564 | 27568047 | rs554790141 | TA | T | 0.3320 | −0.0006 | 0.9994 | 0.9996 |
| 8 | 8_27475320_T_G | 27475320 | 27617803 | rs504038 | G | T | 0.6103 | −0.0031 | 0.9969 | 0.9962 |
| 8 | 8_28612960_T_C | 28612960 | 28755443 | rs73235028 | C | T | 0.0531 | 0.0000 | 1.0000 | 1.0000 |
| 8 | 8_29416526_T_G | 29416526 | 29559009 | rs6986919 | G | T | 0.7546 | 0.0038 | 1.0038 | 1.0058 |
| 8 | 8_29502631_G_A | 29502631 | 29645115 | rs12680857 | A | G | 0.5874 | −0.0090 | 0.9910 | 0.9895 |
| 8 | 8_29509616_A_C | 29509616 | 29652100 | rs9693444 | C | A | 0.6756 | −0.0321 | 0.9684 | 0.9578 |
| 8 | 8_29520078_G_T | 29520078 | 29662562 | rs6981127 | T | G | 0.6470 | −0.0003 | 0.9997 | 0.9996 |
| 8 | 8_29735604_C_T | 29735604 | 29878088 | rs16876096 | T | C | 0.1654 | 0.0091 | 1.0091 | 1.0030 |
| 8 | 8_30165116_T_C | 30165116 | 30307600 | rs10097469 | C | T | 0.0814 | −0.0117 | 0.9884 | 0.9981 |
| 8 | 8_30210274_T_C | 30210274 | 30352758 | rs4349946 | C | T | 0.6697 | −0.0096 | 0.9904 | 0.9872 |
| 8 | 8_31842475_C_T | 31842475 | 31984959 | rs13255716 | T | C | 0.6789 | 0.0079 | 1.0079 | 1.0108 |
| 8 | 8_32015145_G_A | 32015145 | 32157629 | rs1994058 | A | G | 0.8913 | −0.0179 | 0.9823 | 0.9686 |
| 8 | 8_34123592_C_T | 34123592 | 34266074 | rs7840552 | T | C | 0.0198 | −0.0188 | 0.9814 | 0.9993 |
| 8 | 8_34297670_A_T | 34297670 | 34440152 | rs558139335 | T | A | 0.0250 | −0.0235 | 0.9768 | 0.9988 |
| 8 | 8_34955466_G_C | 34955466 | 35097948 | rs17337391 | C | G | 0.0257 | −0.0015 | 0.9985 | 0.9999 |
| 8 | 8_36829456_C_CT | 36829456 | 36971938 | rs61146997 | CTAA | C | 0.2043 | 0.0000 | 1.0000 | 1.0000 |
| | AAA | | | | A | | | | | |
| 8 | 8_36858483_A_G | 36858483 | 37000965 | rs13365225 | G | A | 0.1820 | −0.0504 | 0.9508 | 0.9822 |
| 8 | 8_38565629_T_C | 38565629 | 38708111 | rs10481324 | C | T | 0.3509 | 0.0074 | 1.0074 | 1.0052 |
| 8 | 8_39609628_T_C | 39609628 | 39752109 | rs151305136 | C | T | 0.0217 | −0.0396 | 0.9612 | 0.9983 |
| 8 | 8_39923349_C_A | 39923349 | 40065830 | rs116220892 | A | C | 0.0161 | −0.0258 | 0.9745 | 0.9992 |
| 8 | 8_40179383_C_T | 40179383 | 40321864 | rs4146443 | T | C | 0.3020 | 0.0053 | 1.0053 | 1.0032 |
| 8 | 8_42637683_A_C | 42637683 | 42782540 | rs7825261 | C | A | 0.1982 | −0.0077 | 0.9923 | 0.9970 |
| 8 | 8_51573102_AG_A | 51573102 | 50660542 | rs763199418 | A | AG | 0.2025 | 0.0053 | 1.0053 | 1.0022 |
| 8 | 8_51743692_GTT_ | 51743692 | 50831132 | | GT | GTT | 0.1943 | 0.0069 | 1.0069 | 1.0027 |
| | GT | | | | | | | | | |
| 8 | 8_51788340_G_GA | 51788340 | 50875780 | | GAAA | G | 0.1918 | 0.0040 | 1.0040 | 1.0015 |
| | AACAAAC | | | | CAAA | | | | | |
| | | | | | C | | | | | |
| 8 | 8_51983919_C_T | 51983919 | 51071359 | rs7824633 | T | C | 0.1133 | 0.0000 | 1.0000 | 1.0000 |
| 8 | 8_58757924_T_C | 58757924 | 57845365 | rs2217891 | C | T | 0.3577 | 0.0056 | 1.0056 | 1.0040 |
| 8 | 8_62103694_C_T | 62103694 | 61191135 | rs145226881 | T | C | 0.0235 | −0.0243 | 0.9760 | 0.9989 |
| 8 | 8_65248253_T_G | 65248253 | 64335696 | rs7824601 | G | T | 0.2946 | −0.0050 | 0.9950 | 0.9971 |
| 8 | 8_69403589_C_G | 69403589 | 68491354 | rs55775457 | G | C | 0.0283 | −0.0308 | 0.9697 | 0.9983 |
| 8 | 8_69688844_G_A | 69688844 | 68776609 | rs7833861 | A | G | 0.4535 | −0.0017 | 0.9983 | 0.9985 |
| 8 | 8_74645139_C_T | 74645139 | 73732904 | rs77875439 | T | C | 0.1292 | −0.0048 | 0.9952 | 0.9988 |
| 8 | 8_75857876_G_C | 75857876 | 74945641 | rs11786917 | C | G | 0.3156 | 0.0001 | 1.0001 | 1.0001 |
| 8 | 8_76230943_A_G | 76230943 | 75318708 | rs1511243 | G | A | 0.8282 | 0.0549 | 1.0564 | 1.0957 |
| 8 | 8_76297613_C_A | 76297613 | 75385378 | rs66854431 | A | C | 0.0960 | 0.0093 | 1.0093 | 1.0018 |
| 8 | 8_76315325_C_T | 76315325 | 75403090 | rs72658076 | T | C | 0.2185 | 0.0028 | 1.0028 | 1.0012 |
| 8 | 8_76317377_G_A | 76317377 | 75405142 | rs2980236 | A | G | 0.5427 | 0.0000 | 1.0000 | 1.0000 |
| 8 | 8_76330610_G_A | 76330610 | 75418375 | rs6993283 | A | G | 0.2790 | 0.0000 | 1.0000 | 1.0000 |
| 8 | 8_76333056_C_T | 76333056 | 75420821 | rs72658084 | T | C | 0.0878 | 0.0577 | 1.0594 | 1.0105 |
| 8 | 8_76356580_T_A | 76356580 | 75444345 | rs830787 | A | T | 0.5301 | −0.0072 | 0.9928 | 0.9924 |
| 8 | 8_76373469_C_T | 76373469 | 75461234 | rs2672860 | T | C | 0.3348 | −0.0010 | 0.9990 | 0.9993 |
| 8 | 8_76416112_C_T | 76416112 | 75503877 | rs2926589 | T | C | 0.0603 | 0.0097 | 1.0097 | 1.0012 |
| 8 | 8_76419496_CT_C | 76419496 | 75507261 | rs763756695 | C | CT | 0.3757 | −0.0155 | 0.9846 | 0.9885 |
| 8 | 8_76442665_T_TA | 76442665 | 75530430 | rs34475936 | TA | T | 0.4106 | −0.0004 | 0.9996 | 0.9997 |
| 8 | 8_76475304_T_C | 76475304 | 75563069 | rs2941476 | C | T | 0.1916 | −0.0043 | 0.9957 | 0.9984 |
| 8 | 8_76502796_A_G | 76502796 | 75590561 | rs2977973 | G | A | 0.1160 | 0.0021 | 1.0021 | 1.0005 |
| 8 | 8_76538833_G_A | 76538833 | 75626598 | rs200255906 | A | G | 0.0819 | 0.0076 | 1.0076 | 1.0013 |
| 8 | 8_76900329_T_A | 76900329 | 75988094 | rs12682248 | A | T | 0.7404 | 0.0003 | 1.0003 | 1.0004 |
| 8 | 8_76910809_A_G | 76910809 | 75998574 | rs62517886 | G | A | 0.0617 | −0.0078 | 0.9922 | 0.9990 |
| 8 | 8_77222075_C_G | 77222075 | 76309840 | rs7461846 | G | C | 0.4219 | −0.0004 | 0.9996 | 0.9997 |
| 8 | 8_77920482_G_A | 77920482 | 77008246 | rs7843788 | A | G | 0.5692 | −0.0102 | 0.9899 | 0.9885 |
| 8 | 8_78194627_C_CA | 78194627 | 77282391 | rs5892590 | CAAT | C | 0.7218 | −0.0063 | 0.9937 | 0.9910 |
| | ATGATAAAT | | | | GATA | | | | | |
| | | | | | AAT | | | | | |
| 8 | 8_81359895_G_T | 81359895 | 80447660 | rs10095089 | T | G | 0.1766 | 0.0118 | 1.0119 | 1.0042 |
| 8 | 8_81407157_CAAA | 81407157 | 80494922 | 8:81407157_C | C | CAAA | 0.2504 | 0.0000 | 1.0000 | 1.0000 |
| | _C | | | AAA_C | | | | | | |
| 8 | 8_81543123_A_T | 81543123 | 80630888 | rs7833340 | T | A | 0.0686 | −0.0218 | 0.9784 | 0.9970 |
| 8 | 8_81738475_G_C | 81738475 | 80826240 | rs13272724 | C | G | 0.2257 | 0.0011 | 1.0011 | 1.0005 |
| 8 | 8_81846059_C_A | 81846059 | 80933824 | rs4739773 | A | C | 0.6759 | −0.0089 | 0.9911 | 0.9881 |
| 8 | 8_82226077_G_A | 82226077 | 81313842 | rs74696448 | A | G | 0.0508 | 0.0024 | 1.0024 | 1.0002 |
| 8 | 8_87932278_A_G | 87932278 | 86920050 | rs144776497 | G | A | 0.0163 | 0.0028 | 1.0028 | 1.0001 |
| 8 | 8_87933880_C_T | 87933880 | 86921652 | rs7387763 | T | C | 0.3788 | −0.0023 | 0.9977 | 0.9983 |
| 8 | 8_87953354_G_A | 87953354 | 86941126 | rs13248262 | A | G | 0.5093 | 0.0057 | 1.0057 | 1.0058 |
| 8 | 8_94438986_C_T | 94438986 | 93426758 | rs117879122 | T | C | 0.0289 | −0.0128 | 0.9873 | 0.9993 |
| 8 | 8_95847833_C_CT | 95847833 | 94835605 | rs543122431 | CT | C | 0.2380 | −0.0068 | 0.9932 | 0.9968 |
| 8 | 8_95909498_T_C | 95909498 | 94897270 | rs16917117 | C | T | 0.1841 | −0.0052 | 0.9948 | 0.9981 |
| 8 | 8_96024387_T_A | 96024387 | 95012159 | rs62523112 | A | T | 0.0529 | 0.0122 | 1.0123 | 1.0013 |
| 8 | 8_99022175_C_T | 99022175 | 98009947 | rs117612714 | T | C | 0.0415 | −0.0185 | 0.9817 | 0.9985 |
| 8 | 8_99411087_T_C | 99411087 | 98398859 | rs9297284 | C | T | 0.1745 | −0.0206 | 0.9796 | 0.9929 |
| 8 | 8_99545465_C_T | 99545465 | 98533237 | rs13270534 | T | C | 0.3658 | −0.0021 | 0.9979 | 0.9985 |
| 8 | 8_99553840_A_T | 99553840 | 98541612 | rs10108828 | T | A | 0.4334 | −0.0002 | 0.9998 | 0.9998 |
| 8 | 8_99943768_C_T | 99943768 | 98931540 | rs111593659 | T | C | 0.0110 | 0.0327 | 1.0332 | 1.0007 |
| 8 | 8_101225241_CCC | 101225241 | 100213013 | | CCCGCGG | CCCG | 0.5168 | 0.0042 | 1.0042 | 1.0044 |
| | GCGGCCT_CCCGCG | | | | CCTCCGC | CGGC | | | | |
| | GCCTCCGCGGCCT | | | | GGCCT | CT | | | | |
| 8 | 8_101394894_G_A | 101394894 | 100382666 | rs17424879 | A | G | 0.0572 | 0.0095 | 1.0095 | 1.0011 |
| 8 | 8_102410989_A_C | 102410989 | 101398761 | rs62516992 | C | A | 0.1356 | 0.0000 | 1.0000 | 1.0000 |
| 8 | 8_102475114_G_A | 102475114 | 101462886 | rs1264590 | A | G | 0.7583 | −0.0071 | 0.9929 | 0.9893 |
| 8 | 8_102482149_C_T | 102482149 | 101469921 | rs16867729 | T | C | 0.0256 | 0.0232 | 1.0235 | 1.0012 |
| 8 | 8_102483100_T_C | 102483100 | 101470872 | rs62517052 | C | T | 0.0967 | 0.0062 | 1.0062 | 1.0012 |
| 8 | 8_102499101_T_C | 102499101 | 101486873 | rs509357 | C | T | 0.6749 | −0.0023 | 0.9977 | 0.9969 |
| 8 | 8_102502448_A_G | 102502448 | 101490220 | rs570630 | G | A | 0.2694 | 0.0009 | 1.0009 | 1.0005 |
| 8 | 8_102636399_TCA | 102636399 | 101624171 | 8:102636399_ | T | TCAC | 0.0282 | −0.0002 | 0.9998 | 1.0000 |
| | CAGTA_T | | | TCACAGTA_T | | AGTA | | | | |
| 8 | 8_103750362_A_T | 103750362 | 102738134 | rs62524036 | T | A | 0.0887 | −0.0044 | 0.9956 | 0.9992 |
| 8 | 8_104278384_T_C | 104278384 | 103266156 | rs80346003 | C | T | 0.1214 | 0.0065 | 1.0065 | 1.0016 |
| 8 | 8_106310917_C_T | 106310917 | 105298689 | rs62525396 | T | C | 0.2612 | 0.0000 | 1.0000 | 1.0000 |
| 8 | 8_106321126_G_C | 106321126 | 105308898 | rs74339976 | C | G | 0.5092 | 0.0031 | 1.0031 | 1.0032 |
| 8 | 8_106340292_T_C | 106340292 | 105328064 | rs41324545 | C | T | 0.2417 | 0.0057 | 1.0057 | 1.0028 |
| 8 | 8_106349980_G_G | 106349980 | 105337752 | rs5893739 | GT | G | 0.6510 | 0.0000 | 1.0000 | 1.0000 |
| | T | | | | | | | | | |
| 8 | 8_106358620_A_T | 106358620 | 105346392 | rs12546444 | T | A | 0.1003 | −0.0263 | 0.9740 | 0.9948 |
| 8 | 8_106385049_A_G | 106385049 | 105372821 | rs17283482 | G | A | 0.1628 | 0.0000 | 1.0000 | 1.0000 |
| 8 | 8_106393022_TTA_ | 106393022 | 105380794 | 8:106393022_ | T | TTA | 0.5416 | 0.0043 | 1.0043 | 1.0047 |
| | T | | | TTA_T | | | | | | |
| 8 | 8_107067404_CT_C | 107067404 | 106055176 | rs768648054 | CTT | CT | 0.2454 | 0.0115 | 1.0116 | 1.0057 |
| | TT | | | | | | | | | |
| 8 | 8_107075960_A_G | 107075960 | 106063732 | rs1540419 | G | A | 0.2578 | 0.0055 | 1.0055 | 1.0028 |
| 8 | 8_110884805_CTCT | 110884805 | 109872576 | 8:110884805_ | C | CTCT | 0.0248 | −0.0308 | 0.9697 | 0.9985 |
| | T_C | | | CTCTT_C | | T | | | | |
| 8 | 8_111297253_TA_T | 111297253 | 110285024 | 8:111297253_ | T | TA | 0.6847 | 0.0043 | 1.0043 | 1.0059 |
| | | | | TA_T | | | | | | |
| 8 | 8_113683162_C_T | 113683162 | 112670933 | rs6469434 | T | C | 0.1969 | −0.0047 | 0.9953 | 0.9982 |
| 8 | 8_115343864_CAT | 115343864 | 114331635 | rs149695242 | CATATAG | CATA | 0.1716 | −0.0051 | 0.9949 | 0.9983 |
| | ATAG_CATATAGAT | | | | ATATAG | TAG | | | | |
| | ATAG | | | | | | | | | |
| 8 | 8_116679547_A_G | 116679547 | 115667320 | rs13277568 | G | A | 0.3695 | −0.0095 | 0.9905 | 0.9930 |
| 8 | 8_116934978_G_C | 116934978 | 115922753 | rs6996098 | C | G | 0.8369 | 0.0029 | 1.0029 | 1.0049 |
| 8 | 8_117016733_CAA | 117016733 | 116004508 | rs751476560 | C | CAAA | 0.5897 | 0.0000 | 1.0000 | 1.0000 |
| | A_C | | | | | | | | | |
| 8 | 8_117074659_A_G | 117074659 | 116062434 | rs72681875 | G | A | 0.0432 | 0.0080 | 1.0080 | 1.0007 |
| 8 | 8_117209548_A_G | 117209548 | 116197325 | rs13267382 | G | A | 0.6445 | −0.0217 | 0.9785 | 0.9725 |
| 8 | 8_117234706_C_T | 117234706 | 116222460 | rs17742392 | T | C | 0.0707 | 0.0124 | 1.0125 | 1.0018 |
| 8 | 8_117463949_G_A | 117463949 | 116451711 | rs28695303 | A | G | 0.1983 | −0.0033 | 0.9967 | 0.9987 |
| 8 | 8_117471488_TC_T | 117471488 | 116459250 | rs761872524 | T | TC | 0.1159 | −0.0044 | 0.9956 | 0.9990 |
| 8 | 8_118190620_G_A | 118190620 | 117178381 | rs16889471 | A | G | 0.2082 | 0.0087 | 1.0087 | 1.0036 |
| 8 | 8_118827839_G_A | 118827839 | 117815600 | rs11989122 | A | G | 0.6847 | −0.0035 | 0.9965 | 0.9952 |
| 8 | 8_118881866_T_C | 118881866 | 117869627 | rs10103490 | C | T | 0.3593 | −0.0015 | 0.9985 | 0.9989 |
| 8 | 8_118901928_T_G | 118901928 | 117889689 | rs13280053 | G | T | 0.5102 | 0.0055 | 1.0055 | 1.0056 |
| 8 | 8_120606023_C_T | 120606023 | 119593783 | rs61753744 | T | C | 0.0114 | 0.0181 | 1.0183 | 1.0004 |
| 8 | 8_120862186_A_G | 120862186 | 119849946 | rs62526620 | G | A | 0.1318 | 0.0210 | 1.0212 | 1.0056 |
| 8 | 8_121093721_TTTT | 121093721 | 120081482 | rs199731959 | TTTTTGT | TTTT | 0.6524 | −0.0155 | 0.9846 | 0.9800 |
| | TG_TTTTTGTTTTG | | | | TTTG | TG | | | | |
| 8 | 8_124561917_CT_C | 124561917 | 123549677 | 8:124561917_ | C | CT | 0.4295 | −0.0063 | 0.9937 | 0.9946 |
| | | | | CT_C | | | | | | |
| 8 | 8_124563705_T_C | 124563705 | 123551465 | rs35542655 | C | T | 0.1458 | 0.0040 | 1.0040 | 1.0012 |
| 8 | 8_124571581_G_A | 124571581 | 123559341 | rs12541094 | A | G | 0.4173 | 0.0113 | 1.0114 | 1.0095 |
| 8 | 8_124587952_A_G | 124587952 | 123575712 | rs10091537 | G | A | 0.2523 | 0.0140 | 1.0141 | 1.0071 |
| 8 | 8_124610166_G_A | 124610166 | 123597926 | rs58847541 | A | G | 0.1454 | 0.0002 | 1.0002 | 1.0001 |
| 8 | 8_124612585_C_T | 124612585 | 123600345 | rs62520910 | T | C | 0.1583 | 0.0122 | 1.0123 | 1.0039 |
| 8 | 8_124708820_T_C | 124708820 | 123696580 | rs7004780 | C | T | 0.5340 | 0.0021 | 1.0021 | 1.0022 |
| 8 | 8_124739287_T_C | 124739287 | 123727047 | rs4871410 | C | T | 0.3122 | 0.0000 | 1.0000 | 1.0000 |
| 8 | 8_124739913_T_G | 124739913 | 123727673 | rs7842619 | G | T | 0.3985 | 0.0168 | 1.0169 | 1.0135 |
| 8 | 8_124754173_T_G | 124754173 | 123741933 | rs2008375 | G | T | 0.9158 | 0.0104 | 1.0105 | 1.0192 |
| 8 | 8_124757021_CAA | 124757021 | 123744781 | rs761966047 | C | CAA | 0.3604 | 0.0031 | 1.0031 | 1.0022 |
| | _C | | | | | | | | | |
| 8 | 8_124757130_A_G | 124757130 | 123744890 | rs10105069 | G | A | 0.8856 | 0.0000 | 1.0000 | 1.0000 |
| 8 | 8_124757661_C_T | 124757661 | 123745421 | rs17350191 | T | C | 0.3338 | 0.0093 | 1.0093 | 1.0062 |
| 8 | 8_125586636_A_G | 125586636 | 124574395 | rs4286935 | G | A | 0.1429 | −0.0057 | 0.9943 | 0.9984 |
| 8 | 8_125599071_T_C | 125599071 | 124586830 | rs17372817 | C | T | 0.1240 | −0.0154 | 0.9847 | 0.9962 |
| 8 | 8_127923940_A_G | 127923940 | 126911695 | rs71520605 | G | A | 0.0252 | 0.0028 | 1.0028 | 1.0001 |
| 8 | 8_128208454_G_A | 128208454 | 127196209 | rs7819779 | A | G | 0.2785 | −0.0053 | 0.9947 | 0.9971 |
| 8 | 8 128213561_C_C | 128213561 | 127201316 | rs35961416 | CA | C | 0.4153 | −0.0187 | 0.9815 | 0.9847 |
| | A | | | | | | | | | |
| 8 | 8_128260769_TTTT | 128260769 | 127248524 | rs759883143 | T | TTTT | 0.0149 | 0.0244 | 1.0247 | 1.0007 |
| | A_T | | | | | A | | | | |
| 8 | 8_128305596_G_C | 128305596 | 127293351 | rs283717 | C | G | 0.1521 | 0.0005 | 1.0005 | 1.0002 |
| 8 | 8_128336236_A_G | 128336236 | 127323991 | rs11775799 | G | A | 0.4302 | −0.0053 | 0.9947 | 0.9955 |
| 8 | 8_128351442_A_C | 128351442 | 127339197 | rs56898669 | C | A | 0.3949 | 0.0000 | 1.0000 | 1.0000 |
| 8 | 8_128352730_C_T | 128352730 | 127340485 | rs10087810 | T | C | 0.5744 | 0.0000 | 1.0000 | 1.0000 |
| 8 | 8_128355940_G_A | 128355940 | 127343694 | rs76102714 | A | G | 0.5324 | 0.0000 | 1.0000 | 1.0000 |
| 8 | 8_128366477_G_G | 128366477 | 127354231 | rs71566627 | GAA | G | 0.3242 | −0.0179 | 0.9823 | 0.9885 |
| | AA | | | | | | | | | |
| 8 | 8_128368513_C_T | 128368513 | 127356267 | rs9693995 | T | C | 0.5615 | −0.0046 | 0.9954 | 0.9949 |
| 8 | 8_128370949_C_G | 128370949 | 127358703 | rs12550713 | G | C | 0.4020 | 0.0421 | 1.0430 | 1.0349 |
| 8 | 8_128372172_A_G | 128372172 | 127359926 | rs10096351 | G | A | 0.5446 | 0.0123 | 1.0124 | 1.0135 |
| 8 | 8_128378025_C_T | 128378025 | 127365779 | rs9693143 | T | C | 0.4257 | −0.0171 | 0.9830 | 0.9856 |
| 8 | 8_128383415_C_T | 128383415 | 127371169 | rs117952826 | T | C | 0.0266 | 0.0142 | 1.0143 | 1.0008 |
| 8 | 8_128386556_A_G | 128386556 | 127374310 | rs2121631 | G | A | 0.7179 | 0.0000 | 1.0000 | 1.0000 |
| 8 | 8_128391537_G_C | 128391537 | 127379292 | rs750816 | C | G | 0.1974 | 0.0000 | 1.0000 | 1.0000 |
| 8 | 8_128399222_GA_ | 128399222 | 127386977 | | G | GA | 0.1850 | 0.0000 | 1.0000 | 1.0000 |
| | G | | | | | | | | | |
| 8 | 8_128595821_G_C | 128595821 | 127583576 | rs76671734 | C | G | 0.1734 | −0.0072 | 0.9928 | 0.9975 |
| 8 | 8_128638040_A_G | 128638040 | 127625795 | rs62515760 | G | A | 0.0584 | 0.0032 | 1.0032 | 1.0004 |
| 8 | 8_128689325_T_C | 128689325 | 127677080 | rs7833090 | C | T | 0.2555 | −0.0164 | 0.9837 | 0.9917 |
| 8 | 8_129170126_C_G | 129170126 | 128157880 | rs2608029 | G | C | 0.3372 | 0.0000 | 1.0000 | 1.0000 |
| 8 | 8_129184555_G_A | 129184555 | 128172309 | rs12542202 | A | G | 0.1571 | 0.0183 | 1.0185 | 1.0058 |
| 8 | 8_129191563_ATTT | 129191563 | 128179317 | rs149396369 | A | ATTT | 0.5278 | 0.0000 | 1.0000 | 1.0000 |
| | TTTTT_A | | | | | TTTT | | | | |
| | | | | | | T | | | | |
| 8 | 8_129204262_T_C | 129204262 | 128192016 | rs7017073 | C | T | 0.2155 | 0.0134 | 1.0135 | 1.0058 |
| 8 | 8_129205103_G_A | 129205103 | 128192857 | rs72722767 | A | G | 0.2977 | 0.0054 | 1.0054 | 1.0032 |
| 8 | 8_129229645_A_G | 129229645 | 128217399 | rs7829149 | G | A | 0.3107 | 0.0000 | 1.0000 | 1.0000 |
| 8 | 8_129489903_C_CT | 129489903 | 128477657 | rs34358875 | CTT | C | 0.8614 | −0.0144 | 0.9857 | 0.9755 |
| | T | | | | | | | | | |
| 8 | 8_131968469_T_C | 131968469 | 130956223 | rs6988340 | C | T | 0.9270 | −0.0195 | 0.9807 | 0.9645 |
| 8 | 8_132006257_A_C | 132006257 | 130994011 | rs12676386 | C | A | 0.0246 | 0.0183 | 1.0185 | 1.0009 |
| 8 | 8_132124207_G_A | 132124207 | 131111960 | rs78580956 | A | G | 0.0449 | 0.0022 | 1.0022 | 1.0002 |
| 8 | 8_134224717_C_T | 134224717 | 133212474 | rs7824539 | T | C | 0.1015 | 0.0000 | 1.0000 | 1.0000 |
| 8 | 8_134393849_C_T | 134393849 | 133381606 | rs117057283 | T | C | 0.0123 | 0.0201 | 1.0203 | 1.0005 |
| 8 | 8_134415857_C_T | 134415857 | 133403614 | rs79792639 | T | C | 0.0482 | 0.0044 | 1.0044 | 1.0004 |
| 8 | 8_135117638_TTG_ | 135117638 | 134105395 | 8:135117638_ | T | TTG | 0.4317 | −0.0114 | 0.9887 | 0.9902 |
| | T | | | TTG_T | | | | | | |
| 8 | 8_135551130_G_A | 135551130 | 134538887 | | AA | G | 0.4066 | −0.0037 | 0.9963 | 0.9970 |
| | A | | | | | | | | | |
| 8 | 8_139234794_T_C | 139234794 | 138222551 | rs78827478 | C | T | 0.1290 | 0.0002 | 1.0002 | 1.0001 |
| 8 | 8_139236107_C_A | 139236107 | 138223864 | rs60498507 | A | C | 0.1119 | 0.0172 | 1.0173 | 1.0039 |
| 8 | 8_140464740_T_A | 140464740 | 139452497 | rs2447563 | A | T | 0.9010 | 0.0154 | 1.0155 | 1.0282 |
| 8 | 8_140990881_G_A | 140990881 | 139980672 | rs13279341 | A | G | 0.1398 | −0.0095 | 0.9905 | 0.9974 |
| 8 | 8_143669254_A_G | 143669254 | 142587893 | rs7830152 | G | A | 0.3390 | −0.0141 | 0.9860 | 0.9905 |
| 8 | 8_143670711_GGT | 143670711 | 142589350 | 8:143670711_ | G | GGTG | 0.2001 | −0.0088 | 0.9912 | 0.9965 |
| | GT_G | | | GGTGT_G | | T | | | | |
| 8 | 8_144750677_T_C | 144750677 | 143668507 | rs117423761 | C | T | 0.0256 | 0.0122 | 1.0123 | 1.0006 |
| 9 | 9_1035756_A_AAA | 1035756 | 1035756 | rs150549203 | AAAAAAC | A | 0.1705 | 0.0038 | 1.0038 | 1.0013 |
| | AAAC | | | | | | | | | |
| 9 | 9_1696976_T_C | 1696976 | 1696976 | rs7039610 | C | T | 0.3452 | −0.0014 | 0.9986 | 0.9990 |
| 9 | 9_2124140_C_T | 2124140 | 2124140 | rs10811481 | T | C | 0.1416 | 0.0047 | 1.0047 | 1.0013 |
| 9 | 9_4094321_G_A | 4094321 | 4094321 | rs564275 | A | G | 0.5667 | 0.0042 | 1.0042 | 1.0048 |
| 9 | 9_4167470_G_C | 4167470 | 4167470 | rs10758562 | C | G | 0.2643 | 0.0092 | 1.0092 | 1.0049 |
| 9 | 9_5595577_C_G | 5595577 | 5595577 | rs117152319 | G | C | 0.0159 | 0.0165 | 1.0166 | 1.0005 |
| 9 | 9_5917799_G_A | 5917799 | 5917799 | rs10815309 | A | G | 0.1987 | 0.0027 | 1.0027 | 1.0011 |
| 9 | 9_6002135_T_C | 6002135 | 6002135 | rs10815338 | C | T | 0.2149 | 0.0046 | 1.0046 | 1.0020 |
| 9 | 9_6135481_T_C | 6135481 | 6135481 | rs113396459 | C | T | 0.0187 | −0.0238 | 0.9765 | 0.9991 |
| 9 | 9_6185360_T_C | 6185360 | 6185360 | rs13291323 | C | T | 0.0586 | 0.0046 | 1.0046 | 1.0005 |
| 9 | 9_6445966_TC_T | 6445966 | 6445966 | rs144889864 | T | TC | 0.4814 | 0.0069 | 1.0069 | 1.0067 |
| 9 | 9_6867766_T_C | 6867766 | 6867766 | rs12002100 | C | T | 0.2785 | 0.0000 | 1.0000 | 1.0000 |
| 9 | 9_6880263_A_G | 6880263 | 6880263 | rs10975870 | G | A | 0.2855 | 0.0117 | 1.0118 | 1.0067 |
| 9 | 9_8147463_A_G | 8147463 | 8147463 | rs73640585 | G | A | 0.1313 | −0.0123 | 0.9878 | 0.9968 |
| 9 | 9_8287456_G_C | 8287456 | 8287456 | rs141167506 | C | G | 0.0118 | −0.0200 | 0.9802 | 0.9995 |
| 9 | 9_8415317_T_C | 8415317 | 8415317 | rs61454491 | C | T | 0.0290 | 0.0368 | 1.0375 | 1.0022 |
| 9 | 9_8545593_G_A | 8545593 | 8545593 | rs76007978 | A | G | 0.0126 | −0.0595 | 0.9422 | 0.9985 |
| 9 | 9_9017452_G_A | 9017452 | 9017452 | rs140019155 | A | G | 0.0413 | −0.0006 | 0.9994 | 1.0000 |
| 9 | 9_10270636_A_G | 10270636 | 10270636 | rs1322155 | G | A | 0.6771 | −0.0066 | 0.9934 | 0.9911 |
| 9 | 9_11896699_A_G | 11896699 | 11896699 | rs12553199 | G | A | 0.0297 | 0.0078 | 1.0078 | 1.0005 |
| 9 | 9_12021101_A_G | 12021101 | 12021101 | rs75934379 | G | A | 0.0195 | −0.0045 | 0.9955 | 0.9998 |
| 9 | 9_12141554_G_A | 12141554 | 12141554 | rs78752415 | A | G | 0.0169 | −0.0400 | 0.9608 | 0.9987 |
| 9 | 9_13488564_C_G | 13488564 | 13488565 | rs7856695 | G | C | 0.4329 | 0.0004 | 1.0004 | 1.0003 |
| 9 | 9_13592493_A_T | 13592493 | 13592494 | rs5009186 | T | A | 0.7545 | 0.0049 | 1.0049 | 1.0074 |
| 9 | 9_13942940_G_A | 13942940 | 13942941 | rs77457752 | A | G | 0.1303 | −0.0225 | 0.9778 | 0.9942 |
| 9 | 9_14021151_T_C | 14021151 | 14021152 | rs35473056 | C | T | 0.1442 | 0.0121 | 1.0122 | 1.0035 |
| 9 | 9_14021276_G_C | 14021276 | 14021277 | rs77875888 | C | G | 0.0658 | −0.0002 | 0.9998 | 1.0000 |
| 9 | 9_15044787_C_T | 15044787 | 15044789 | rs72715698 | T | C | 0.0475 | 0.0065 | 1.0065 | 1.0006 |
| 9 | 9_16046463_A_G | 16046463 | 16046465 | rs3008694 | G | A | 0.2861 | 0.0132 | 1.0133 | 1.0076 |
| 9 | 9_16093549_G_T | 16093549 | 16093551 | rs62552203 | T | G | 0.1760 | −0.0045 | 0.9955 | 0.9984 |
| 9 | 9_16337782_T_C | 16337782 | 16337784 | rs10962385 | C | T | 0.2976 | 0.0042 | 1.0042 | 1.0025 |
| 9 | 9_16838211_A_G | 16838211 | 16838213 | rs4995398 | G | A | 0.2828 | −0.0010 | 0.9990 | 0.9994 |
| 9 | 9_16841628_A_G | 16841628 | 16841630 | rs10810643 | G | A | 0.2293 | −0.0037 | 0.9963 | 0.9983 |
| 9 | 9_16844142_G_GT | 16844142 | 16844144 | rs5896709 | GT | G | 0.2208 | −0.0117 | 0.9884 | 0.9949 |
| 9 | 9_18817372_C_T | 18817372 | 18817374 | rs7873880 | T | C | 0.6976 | −0.0056 | 0.9944 | 0.9922 |
| 9 | 9_20812064_G_A | 20812064 | 20812065 | rs74354652 | A | G | 0.0152 | −0.0385 | 0.9622 | 0.9989 |
| 9 | 9_21631421_A_G | 21631421 | 21631422 | rs117276645 | G | A | 0.0221 | −0.0257 | 0.9746 | 0.9989 |
| 9 | 9_21950780_A_T | 21950780 | 21950781 | rs112109214 | T | A | 0.0237 | −0.0011 | 0.9989 | 0.9999 |
| 9 | 9_21964882_CAAA | 21964882 | 21964883 | rs745322029 | C | CAAA | 0.3184 | 0.0281 | 1.0285 | 1.0182 |
| | A_C | | | | | A | | | | |
| 9 | 9_22003223_C_T | 22003223 | 22003224 | rs3217992 | T | C | 0.3813 | −0.0133 | 0.9868 | 0.9900 |
| 9 | 9_22005330_T_G | 22005330 | 22005331 | rs3217986 | G | T | 0.0813 | 0.0014 | 1.0014 | 1.0002 |
| 9 | 9_22011083_G_C | 22011083 | 22011084 | rs76959412 | C | G | 0.0166 | 0.0350 | 1.0356 | 1.0012 |
| 9 | 9_22041998_C_G | 22041998 | 22041999 | rs17694493 | G | C | 0.1393 | 0.0013 | 1.0013 | 1.0004 |
| 9 | 9_26040096_G_A | 26040096 | 26040098 | rs35323787 | A | G | 0.0464 | 0.0277 | 1.0281 | 1.0026 |
| 9 | 9_29638167_G_T | 29638167 | 29638169 | rs546929 | T | G | 0.0872 | 0.0033 | 1.0033 | 1.0006 |
| 9 | 9_29849357_G_C | 29849357 | 29849359 | rs115652823 | C | G | 0.0115 | 0.0004 | 1.0004 | 1.0000 |
| 9 | 9_32455674_A_G | 32455674 | 32455676 | rs1133071 | G | A | 0.3118 | −0.0093 | 0.9907 | 0.9942 |
| 9 | 9_32523737_T_C | 32523737 | 32523739 | rs11795343 | C | T | 0.4051 | 0.0000 | 1.0000 | 1.0000 |
| 9 | 9_34108800_T_A | 34108800 | 34108802 | rs10738923 | A | T | 0.7218 | −0.0020 | 0.9980 | 0.9971 |
| 9 | 9_34130435_G_A | 34130435 | 34130437 | rs61237993 | A | G | 0.1289 | 0.0023 | 1.0023 | 1.0006 |
| 9 | 9_36209082_G_A | 36209082 | 36209085 | rs62541769 | A | G | 0.0523 | −0.0121 | 0.9880 | 0.9987 |
| 9 | 9_36921921_C_A | 36921921 | 36921924 | rs4880037 | A | C | 0.7525 | 0.0000 | 1.0000 | 1.0000 |
| 9 | 9_36928288_T_C | 36928288 | 36928291 | rs4880038 | C | T | 0.5349 | 0.0096 | 1.0096 | 1.0103 |
| 9 | 9_36931653_C_G | 36931653 | 36931656 | rs4369064 | G | C | 0.2502 | −0.0006 | 0.9994 | 0.9997 |
| 9 | 9_36951035_A_G | 36951035 | 36951038 | rs10511936 | G | A | 0.2432 | −0.0034 | 0.9966 | 0.9983 |
| 9 | 9_36974535_G_A | 36974535 | 36974538 | rs58210769 | A | G | 0.2266 | −0.0040 | 0.9960 | 0.9982 |
| 9 | 9_71922028_C_T | 71922028 | 69307112 | rs117355427 | T | C | 0.0268 | −0.0260 | 0.9743 | 0.9986 |
| 9 | 9_72519703_G_A | 72519703 | 69904787 | rs11140844 | A | G | 0.7393 | 0.0049 | 1.0049 | 1.0073 |
| 9 | 9_73394053_C_G | 73394053 | 70779137 | rs1328145 | G | C | 0.9425 | −0.0082 | 0.9918 | 0.9847 |
| 9 | 9_73874330_G_A | 73874330 | 71259414 | rs149472628 | A | G | 0.0397 | 0.0080 | 1.0080 | 1.0006 |
| 9 | 9_73910400_A_G | 73910400 | 71295484 | rs72733896 | G | A | 0.0554 | 0.0139 | 1.0140 | 1.0016 |
| 9 | 9_74794895_G_T | 74794895 | 72179979 | rs10869141 | T | G | 0.2132 | −0.0087 | 0.9913 | 0.9963 |
| 9 | 9_74910180_C_A | 74910180 | 72295264 | rs12238801 | A | C | 0.1740 | 0.0096 | 1.0096 | 1.0034 |
| 9 | 9_77752967_A_G | 77752967 | 75138051 | rs1009282 | G | A | 0.5733 | 0.0108 | 1.0109 | 1.0125 |
| 9 | 9_78308301_G_A | 78308301 | 75693385 | rs7032375 | A | G | 0.1046 | 0.0228 | 1.0231 | 1.0048 |
| 9 | 9_78910108_A_G | 78910108 | 76295192 | rs13283901 | G | A | 0.4401 | −0.0021 | 0.9979 | 0.9982 |
| 9 | 9_78934242_A_T | 78934242 | 76319326 | rs56383794 | T | A | 0.1247 | 0.0026 | 1.0026 | 1.0006 |
| 9 | 9_78969417_C_T | 78969417 | 76354501 | rs72745133 | T | C | 0.2807 | 0.0005 | 1.0005 | 1.0003 |
| 9 | 9_78971232_G_A | 78971232 | 76356316 | rs10869765 | A | G | 0.5413 | −0.0063 | 0.9937 | 0.9932 |
| 9 | 9_80148344_C_A | 80148344 | 77533428 | rs13287722 | A | C | 0.1472 | −0.0005 | 0.9995 | 0.9999 |
| 9 | 9_80151268_T_G | 80151268 | 77536352 | rs10869939 | G | T | 0.2444 | −0.0093 | 0.9907 | 0.9955 |
| 9 | 9_80716880_A_C | 80716880 | 78101964 | rs4877178 | C | A | 0.4793 | −0.0040 | 0.9960 | 0.9962 |
| 9 | 9_82584696_GA_G | 82584696 | 79969781 | 9:82584696_G | G | GA | 0.0325 | −0.0150 | 0.9851 | 0.9990 |
| | | | | A_G | | | | | | |
| 9 | 9_85950924_C_T | 85950924 | 83336009 | rs72618135 | T | C | 0.0923 | 0.0139 | 1.0140 | 1.0026 |
| 9 | 9_86065360_T_C | 86065360 | 83450445 | rs28719707 | C | T | 0.0807 | −0.0011 | 0.9989 | 0.9998 |
| 9 | 9_87736422_G_A | 87736422 | 85121507 | rs11140866 | A | G | 0.4293 | 0.0033 | 1.0033 | 1.0028 |
| 9 | 9_87782211_T_C | 87782211 | 85167296 | rs665889 | C | T | 0.5094 | 0.0131 | 1.0132 | 1.0135 |
| 9 | 9_87889877_TC_T | 87889877 | 85274962 | 9:87889877_T | T | TC | 0.0302 | 0.0110 | 1.0111 | 1.0007 |
| | | | | C_T | | | | | | |
| 9 | 9_88011274_C_G | 88011274 | 85396359 | rs28657389 | G | C | 0.0875 | 0.0000 | 1.0000 | 1.0000 |
| 9 | 9_89438251_A_G | 89438251 | 86823336 | rs7847644 | G | A | 0.3477 | 0.0025 | 1.0025 | 1.0017 |
| 9 | 9_89440695_C_T | 89440695 | 86825780 | rs10868490 | T | C | 0.3995 | 0.0012 | 1.0012 | 1.0010 |
| 9 | 9_90274681_C_A | 90274681 | 87659766 | rs6560013 | A | C | 0.1114 | 0.0039 | 1.0039 | 1.0009 |
| 9 | 9_92263290_CAA_ | 92263290 | 89648375 | 9:92263290_C | C | CAA | 0.1546 | −0.0092 | 0.9908 | 0.9972 |
| | C | | | AA_C | | | | | | |
| 9 | 9_92848755_A_G | 92848755 | 90086473 | rs11792411 | G | A | 0.4743 | 0.0000 | 1.0000 | 1.0000 |
| 9 | 9_94229859_T_C | 94229859 | 91467577 | rs117362578 | C | T | 0.0148 | 0.0064 | 1.0064 | 1.0002 |
| 9 | 9_94260641_T_C | 94260641 | 91498359 | rs149668079 | C | T | 0.0149 | 0.0149 | 1.0150 | 1.0004 |
| 9 | 9_94260650_G_A | 94260650 | 91498368 | rs57587931 | A | G | 0.0447 | 0.0140 | 1.0141 | 1.0013 |
| 9 | 9_95751862_A_G | 95751862 | 92989580 | rs4744162 | G | A | 0.5401 | −0.0043 | 0.9957 | 0.9954 |
| 9 | 9_97770605_C_A | 97770605 | 95008323 | rs7862618 | A | C | 0.0145 | −0.0506 | 0.9507 | 0.9986 |
| 9 | 9_98022968_T_A | 98022968 | 95260686 | rs7021497 | A | T | 0.4322 | 0.0039 | 1.0039 | 1.0034 |
| 9 | 9_98329088_C_T | 98329088 | 95566806 | rs28675274 | T | C | 0.0853 | 0.0044 | 1.0044 | 1.0008 |
| 9 | 9_98362587_T_C | 98362587 | 95600305 | rs10120432 | C | T | 0.0940 | 0.0238 | 1.0241 | 1.0045 |
| 9 | 9_98474673_T_A | 98474673 | 95712391 | rs60806471 | A | T | 0.1234 | 0.0095 | 1.0095 | 1.0024 |
| 9 | 9_100806287_CTT_ | 100806287 | 98044005 | rs762895782 | C | CTT | 0.2575 | 0.0028 | 1.0028 | 1.0014 |
| | C | | | | | | | | | |
| 9 | 9_107013881_C_T | 107013881 | 104251600 | rs4743702 | T | C | 0.5306 | −0.0046 | 0.9954 | 0.9951 |
| 9 | 9_107100197_G_G | 107100197 | 104337916 | | GTT | G | 0.0268 | 0.0248 | 1.0251 | 1.0013 |
| | TT | | | | | | | | | |
| 9 | 9_107637306_A_G | 107637306 | 104875025 | rs59237458 | G | A | 0.4372 | 0.0000 | 1.0000 | 1.0000 |
| 9 | 9_107872109_C_G | 107872109 | 105109828 | rs55659643 | G | C | 0.0131 | 0.0032 | 1.0032 | 1.0001 |
| 9 | 9_107958636_C_G | 107958636 | 105196355 | rs327970 | G | C | 0.1796 | 0.0059 | 1.0059 | 1.0021 |
| 9 | 9_107979858_A_C | 107979858 | 105217577 | rs399707 | C | A | 0.1784 | 0.0036 | 1.0036 | 1.0013 |
| 9 | 9_110275369_T_C | 110275369 | 107513088 | rs149346572 | C | T | 0.0179 | 0.0175 | 1.0177 | 1.0006 |
| 9 | 9_110275852_A_T | 110275852 | 107513571 | rs10816524 | T | A | 0.0725 | 0.0227 | 1.0230 | 1.0033 |
| 9 | 9_110300994_A_G | 110300994 | 107538713 | rs4979065 | G | A | 0.4992 | 0.0034 | 1.0034 | 1.0034 |
| 9 | 9_110303463_A_G | 110303463 | 107541182 | rs2185857 | G | A | 0.2716 | 0.0005 | 1.0005 | 1.0003 |
| 9 | 9_110303808_TAA | 110303808 | 107541527 | 9:110303808_ | T | TAA | 0.2065 | 0.0461 | 1.0472 | 1.0196 |
| | _T | | | TAA_T | | | | | | |
| 9 | 9_110304319_A_T | 110304319 | 107542038 | rs3956 | T | A | 0.1044 | 0.0050 | 1.0050 | 1.0010 |
| 9 | 9_110305205_CT_C | 110305205 | 107542924 | 9:110305205_ | C | CT | 0.2667 | 0.0000 | 1.0000 | 1.0000 |
| | | | | CT_C | | | | | | |
| 9 | 9_110429664_A_G | 110429664 | 107667383 | rs77233150 | G | A | 0.0574 | −0.0096 | 0.9904 | 0.9989 |
| 9 | 9_110458648_A_C | 110458648 | 107696367 | rs10123702 | C | A | 0.1482 | −0.0082 | 0.9918 | 0.9976 |
| 9 | 9_110462837_G_A | 110462837 | 107700556 | rs4979255 | A | G | 0.1678 | −0.0002 | 0.9998 | 0.9999 |
| 9 | 9_110479474_T_A | 110479474 | 107717193 | rs59221680 | A | T | 0.3645 | −0.0027 | 0.9973 | 0.9980 |
| 9 | 9_110484484_T_C | 110484484 | 107722203 | rs11792761 | C | T | 0.2913 | −0.0030 | 0.9970 | 0.9983 |
| 9 | 9_110484770_T_C | 110484770 | 107722489 | rs1962079 | C | T | 0.4170 | −0.0032 | 0.9968 | 0.9973 |
| 9 | 9_110773040_C_T | 110773040 | 108010759 | rs60778117 | T | C | 0.1826 | 0.0000 | 1.0000 | 1.0000 |
| 9 | 9_110821050_C_T | 110821050 | 108058769 | rs10979183 | T | C | 0.4054 | 0.0058 | 1.0058 | 1.0047 |
| 9 | 9_110837073_A_G | 110837073 | 108074792 | rs10816625 | G | A | 0.0630 | 0.0720 | 1.0747 | 1.0094 |
| 9 | 9_110837176_C_T | 110837176 | 108074895 | rs13294895 | T | C | 0.1750 | 0.0368 | 1.0375 | 1.0132 |
| 9 | 9_110849525_G_T | 110849525 | 108087244 | rs7848334 | T | G | 0.5977 | 0.0067 | 1.0067 | 1.0081 |
| 9 | 9_110869006_A_A | 110869006 | 108106726 | rs10644017 | ATATAT | A | 0.4163 | 0.0071 | 1.0071 | 1.0059 |
| | TATAT | | | | | | | | | |
| 9 | 9_110882390_T_C | 110882390 | 108120110 | rs667052 | C | T | 0.4689 | 0.0000 | 1.0000 | 1.0000 |
| 9 | 9_110885479_C_T | 110885479 | 108123199 | rs630965 | T | C | 0.6222 | 0.0634 | 1.0655 | 1.0831 |
| 9 | 9_110902100_T_C | 110902100 | 108139820 | rs575270 | C | T | 0.5870 | 0.0021 | 1.0021 | 1.0025 |
| 9 | 9_110919211_C_T | 110919211 | 108156931 | rs189368445 | T | C | 0.0264 | −0.0147 | 0.9854 | 0.9992 |
| 9 | 9_110920305_G_A | 110920305 | 108158025 | rs7850979 | A | G | 0.3122 | 0.0016 | 1.0016 | 1.0010 |
| 9 | 9_110921715_G_A | 110921715 | 108159435 | rs10119131 | A | G | 0.2387 | 0.0000 | 1.0000 | 1.0000 |
| 9 | 9_110925407_C_A | 110925407 | 108163127 | rs630611 | A | C | 0.6230 | 0.0000 | 1.0000 | 1.0000 |
| 9 | 9_110988346_A_G | 110988346 | 108226066 | rs7043432 | G | A | 0.2052 | 0.0040 | 1.0040 | 1.0016 |
| 9 | 9_111059697_T_C | 111059697 | 108297417 | rs55638805 | C | T | 0.1357 | 0.0012 | 1.0012 | 1.0003 |
| 9 | 9_111329010_A_G | 111329010 | 108566730 | rs10125630 | G | A | 0.1935 | 0.0002 | 1.0002 | 1.0001 |
| 9 | 9_111859015_C_T | 111859015 | 109096735 | rs142211233 | T | C | 0.0101 | −0.0130 | 0.9871 | 0.9997 |
| 9 | 9_111927669_C_T | 111927669 | 109165389 | rs62576372 | T | C | 0.0256 | −0.0303 | 0.9702 | 0.9985 |
| 9 | 9_112053123_T_C | 112053123 | 109290843 | rs55700539 | C | T | 0.1575 | −0.0011 | 0.9989 | 0.9997 |
| 9 | 9_112606832_A_G | 112606832 | 109844552 | rs11789107 | G | A | 0.1196 | 0.0032 | 1.0032 | 1.0008 |
| 9 | 9_112607249_A_G | 112607249 | 109844969 | rs78904538 | G | A | 0.1643 | 0.0025 | 1.0025 | 1.0008 |
| 9 | 9_114700288_G_A | 114700288 | 111938008 | rs79312461 | A | G | 0.0351 | −0.0152 | 0.9849 | 0.9989 |
| 9 | 9_114965309_C_CT | 114965309 | 112203029 | rs35116137 | CT | C | 0.4294 | 0.0012 | 1.0012 | 1.0010 |
| 9 | 9_115202610_G_A | 115202610 | 112440330 | rs10123584 | A | G | 0.7014 | 0.0000 | 1.0000 | 1.0000 |
| 9 | 9_115737007_G_A | 115737007 | 112974727 | rs12377906 | A | G | 0.2157 | −0.0004 | 0.9996 | 0.9998 |
| 9 | 9_115737606_A_T | 115737606 | 112975326 | rs4979199 | T | A | 0.2706 | −0.0056 | 0.9944 | 0.9970 |
| 9 | 9_116188725_G_T | 116188725 | 113426445 | rs7870912 | T | G | 0.1881 | −0.0091 | 0.9909 | 0.9966 |
| 9 | 9_117802247_T_A | 117802247 | 115039968 | rs10982505 | A | T | 0.0464 | 0.0043 | 1.0043 | 1.0004 |
| 9 | 9_118408986_G_C | 118408986 | 115646707 | rs62575330 | C | G | 0.0759 | 0.0052 | 1.0052 | 1.0008 |
| 9 | 9_119153708_C_T | 119153708 | 116391429 | rs74363364 | T | C | 0.0598 | 0.0002 | 1.0002 | 1.0000 |
| 9 | 9_119254872_C_T | 119254872 | 116492593 | rs13296745 | T | C | 0.2291 | −0.0023 | 0.9977 | 0.9989 |
| 9 | 9_119308184_G_T | 119308184 | 116545905 | rs7029693 | T | G | 0.3643 | −0.0078 | 0.9922 | 0.9943 |
| 9 | 9_119313486_A_G | 119313486 | 116551207 | rs1895062 | G | A | 0.4087 | −0.0145 | 0.9856 | 0.9883 |
| 9 | 9_119487573_A_T | 119487573 | 116725294 | rs894206 | T | A | 0.2536 | −0.0001 | 0.9999 | 0.9999 |
| 9 | 9_119863594_C_A | 119863594 | 117101315 | rs79282494 | A | C | 0.2170 | 0.0000 | 1.0000 | 1.0000 |
| 9 | 9_123311473_C_T | 123311473 | 120549195 | rs77565290 | T | C | 0.0651 | 0.0145 | 1.0146 | 1.0019 |
| 9 | 9_123651703_CA_ | 123651703 | 120889425 | 9:123651703_ | C | CA | 0.5931 | −0.0052 | 0.9948 | 0.9939 |
| | C | | | CA_C | | | | | | |
| 9 | 9_123896570_TA_T | 123896570 | 121134292 | rs200665428 | T | TA | 0.6521 | −0.0083 | 0.9917 | 0.9892 |
| 9 | 9_124330970_T_C | 124330970 | 121568691 | rs10985320 | C | T | 0.1644 | 0.0046 | 1.0046 | 1.0015 |
| 9 | 9_126741702_A_G | 126741702 | 123979423 | rs116991872 | G | A | 0.0178 | 0.0277 | 1.0281 | 1.0010 |
| 9 | 9_126837432_G_A | 126837432 | 124075153 | rs10986200 | A | G | 0.0603 | 0.0035 | 1.0035 | 1.0004 |
| 9 | 9_129346682_T_C | 129346682 | 126584403 | rs6478744 | C | T | 0.3584 | −0.0002 | 0.9998 | 0.9999 |
| 9 | 9_129399562_A_G | 129399562 | 126637283 | rs62578132 | G | A | 0.0857 | 0.0013 | 1.0013 | 1.0002 |
| 9 | 9_129402567_G_A | 129402567 | 126640288 | rs12342373 | A | G | 0.0927 | 0.0026 | 1.0026 | 1.0005 |
| 9 | 9_129409062_C_A | 129409062 | 126646783 | rs4837102 | A | C | 0.0436 | −0.0065 | 0.9935 | 0.9994 |
| 9 | 9_129424719_A_G | 129424719 | 126662440 | rs3861871 | G | A | 0.4577 | −0.0157 | 0.9844 | 0.9858 |
| 9 | 9_129426469_G_T | 129426469 | 126664190 | rs10429515 | T | G | 0.1074 | −0.0125 | 0.9876 | 0.9973 |
| 9 | 9_129687836_GAA | 129687836 | 126925557 | 9:129687836_ | G | GAA | 0.4405 | −0.0004 | 0.9996 | 0.9996 |
| | _G | | | GAA_G | | | | | | |
| 9 | 9_130164993_C_T | 130164993 | 127402714 | rs1138739 | T | C | 0.2263 | −0.0026 | 0.9974 | 0.9988 |
| 9 | 9_130488607_G_A | 130488607 | 127726328 | rs142193455 | A | G | 0.0163 | −0.0148 | 0.9853 | 0.9995 |
| 9 | 9_131217803_A_G | 131217803 | 128455524 | rs55969420 | G | A | 0.1025 | −0.0061 | 0.9939 | 0.9988 |
| 9 | 9_133069741_C_A | 133069741 | 130307462 | rs143378550 | A | C | 0.0264 | 0.0422 | 1.0431 | 1.0023 |
| 9 | 9_133888994_T_C | 133888994 | 131013607 | rs115491381 | C | T | 0.0707 | 0.0068 | 1.0068 | 1.0010 |
| 9 | 9_135871155_T_C | 135871155 | 132995768 | rs3011266 | C | T | 0.7426 | −0.0027 | 0.9973 | 0.9960 |
| 9 | 9_136146597_C_T | 136146597 | 133271182 | rs550057 | T | C | 0.2727 | 0.0078 | 1.0078 | 1.0043 |
| 9 | 9_136149830_G_A | 136149830 | 133274414 | rs532436 | A | G | 0.2038 | 0.0001 | 1.0001 | 1.0000 |
| 9 | 9_136336804_C_T | 136336804 | 133471682 | rs41307428 | T | C | 0.0456 | −0.0136 | 0.9865 | 0.9988 |
| 9 | 9_136350150_T_C | 136350150 | 133485028 | rs3094324 | C | T | 0.7391 | −0.0097 | 0.9903 | 0.9858 |
| 9 | 9_136361351_TTGT | 136361351 | 133496229 | 9:136361351_ | TTG | TTGT | 0.4886 | −0.0022 | 0.9978 | 0.9979 |
| | G_TTG | | | TTG_T | | G | | | | |
| 9 | 9_138532484_T_A | 138532484 | 135640638 | rs62577654 | A | T | 0.0535 | 0.0379 | 1.0386 | 1.0041 |
| 9 | 9_140488857_C_G | 140488857 | 137594405 | 9:140488857_ | G | C | 0.3502 | −0.0064 | 0.9936 | 0.9955 |
| | | | | C_G | | | | | | |
| 10 | 10_2135126_T_C | 2135126 | 2092932 | rs11594194 | C | T | 0.2071 | 0.0000 | 1.0000 | 1.0000 |
| 10 | 10_5401308_T_C | 5401308 | 5359345 | rs7081234 | C | T | 0.5155 | 0.0035 | 1.0035 | 1.0036 |
| 10 | 10_5760276_C_T | 5760276 | 5718313 | rs35585360 | T | C | 0.4766 | −0.0015 | 0.9985 | 0.9986 |
| 10 | 10_5794652_A_G | 5794652 | 5752689 | rs55910451 | G | A | 0.2137 | 0.0112 | 1.0113 | 1.0048 |
| 10 | 10_5871493_T_G | 5871493 | 5829530 | rs12763451 | G | T | 0.4373 | −0.0069 | 0.9931 | 0.9940 |
| 10 | 10_5872086_C_T | 5872086 | 5830123 | rs112168619 | T | C | 0.2031 | 0.0063 | 1.0063 | 1.0026 |
| 10 | 10_5942711_G_A | 5942711 | 5900748 | rs34825212 | A | G | 0.1397 | −0.0085 | 0.9915 | 0.9976 |
| 10 | 10_6151327_C_A | 6151327 | 6109364 | rs7916878 | A | C | 0.5254 | 0.0048 | 1.0048 | 1.0051 |
| 10 | 10_8215279_C_A | 8215279 | 8173316 | rs67549624 | A | C | 0.3135 | 0.0026 | 1.0026 | 1.0016 |
| 10 | 10_8357966_T_C | 8357966 | 8316003 | rs1819622 | C | T | 0.6198 | −0.0077 | 0.9923 | 0.9905 |
| 10 | 10_8437546_G_A | 8437546 | 8395583 | rs2787136 | A | G | 0.5271 | 0.0009 | 1.0009 | 1.0009 |
| 10 | 10_8697128_G_A | 8697128 | 8655165 | rs74433033 | A | G | 0.0354 | 0.0101 | 1.0102 | 1.0007 |
| 10 | 10_9101963_T_TAT | 9101963 | 9060000 | rs796812026 | TATATAT | T | 0.6538 | −0.0029 | 0.9971 | 0.9962 |
| | ATATATATAC | | | | ATATAC | | | | | |
| 10 | 10_9107670_G_A | 9107670 | 9065707 | rs34724453 | A | G | 0.1169 | 0.0205 | 1.0207 | 1.0048 |
| 10 | 10_9203158_GTT_ | 9203158 | 9161195 | | GT | GTT | 0.4642 | 0.0003 | 1.0003 | 1.0003 |
| | GT | | | | | | | | | |
| 10 | 10_11186952_C_G | 11186952 | 11144989 | rs7919542 | G | C | 0.1130 | 0.0041 | 1.0041 | 1.0009 |
| 10 | 10_11198434_G_C | 11198434 | 11156471 | rs666871 | C | G | 0.2607 | 0.0097 | 1.0097 | 1.0051 |
| 10 | 10_11242078_TGA | 11242078 | 11200115 | 10:11242078_ | T | TGAG | 0.0375 | 0.0121 | 1.0122 | 1.0009 |
| | GAA_T | | | TGAGAA_T | | AA | | | | |
| 10 | 10_13560152_A_G | 13560152 | 13518152 | rs118012425 | G | A | 0.0141 | 0.0657 | 1.0679 | 1.0019 |
| 10 | 10_13907236_T_C | 13907236 | 13865236 | rs10906522 | C | T | 0.4691 | 0.0123 | 1.0124 | 1.0116 |
| 10 | 10_13917699_C_T | 13917699 | 13875699 | rs11258686 | T | C | 0.3629 | −0.0007 | 0.9993 | 0.9995 |
| 10 | 10_13947651_T_C | 13947651 | 13905651 | rs12776420 | C | T | 0.3592 | −0.0020 | 0.9980 | 0.9986 |
| 10 | 10_14289822_G_A | 14289822 | 14247823 | rs146106883 | A | G | 0.0101 | 0.0440 | 1.0450 | 1.0009 |
| 10 | 10_14332199_G_A | 14332199 | 14290200 | rs148393137 | A | G | 0.0276 | 0.0299 | 1.0304 | 1.0017 |
| 10 | 10_17605117_A_G | 17605117 | 17563118 | rs2186113 | G | A | 0.5546 | −0.0048 | 0.9952 | 0.9947 |
| 10 | 10_19552471_C_T | 19552471 | 19263542 | rs12356390 | T | C | 0.1461 | −0.0117 | 0.9884 | 0.9966 |
| 10 | 10_20077518_G_A | 20077518 | 19788589 | rs77016942 | A | G | 0.0192 | −0.0199 | 0.9803 | 0.9992 |
| 10 | 10_20390278_G_A | 20390278 | 20101349 | rs7068493 | A | G | 0.0283 | −0.0104 | 0.9897 | 0.9994 |
| 10 | 10_21772735_CA_ | 21772735 | 21483806 | 10:21772735_ | C | CA | 0.3823 | 0.0000 | 1.0000 | 1.0000 |
| | C | | | CA_C | | | | | | |
| 10 | 10_21811487_G_A | 21811487 | 21522558 | rs11012730 | A | G | 0.3781 | 0.0000 | 1.0000 | 1.0000 |
| 10 | 10_21824727_G_A | 21824727 | 21535798 | rs10828249 | A | G | 0.3615 | 0.0234 | 1.0237 | 1.0172 |
| 10 | 10_22032942_A_G | 22032942 | 21744013 | rs7072776 | G | A | 0.7085 | −0.0010 | 0.9990 | 0.9986 |
| 10 | 10_22037809_A_G | 22037809 | 21748880 | rs6482190 | G | A | 0.7107 | −0.0026 | 0.9974 | 0.9963 |
| 10 | 10_22043407_A_G | 22043407 | 21754478 | rs17409138 | G | A | 0.1913 | 0.0000 | 1.0000 | 1.0000 |
| 10 | 10_22216608_T_C | 22216608 | 21927679 | rs72797470 | C | T | 0.0211 | 0.0378 | 1.0385 | 1.0016 |
| 10 | 10_22233099_G_G | 22233099 | 21944170 | rs11411342 | GT | G | 0.6056 | 0.0000 | 1.0000 | 1.0000 |
| | T | | | | | | | | | |
| 10 | 10_22294687_C_G | 22294687 | 22005758 | rs7069830 | G | C | 0.6923 | 0.0000 | 1.0000 | 1.0000 |
| 10 | 10_22432882_C_T | 22432882 | 22143953 | rs56346589 | T | C | 0.0194 | 0.0073 | 1.0073 | 1.0003 |
| 10 | 10_22477776_ACC | 22477776 | 22188847 | rs762131501 | A | ACC | 0.0202 | 0.0637 | 1.0658 | 1.0027 |
| | _A | | | | | | | | | |
| 10 | 10_22487991_T_C | 22487991 | 22199062 | rs72812786 | C | T | 0.0187 | 0.0081 | 1.0081 | 1.0003 |
| 10 | 10_22718486_G_T | 22718486 | 22429557 | rs75723675 | T | G | 0.0528 | 0.0050 | 1.0050 | 1.0005 |
| 10 | 10_22861490_A_C | 22861490 | 22572561 | rs10764337 | C | A | 0.9370 | 0.0056 | 1.0056 | 1.0106 |
| 10 | 10_22862487_C_A | 22862487 | 22573558 | rs11013052 | A | C | 0.3118 | 0.0036 | 1.0036 | 1.0023 |
| 10 | 10_22863028_CAA | 22863028 | 22574099 | 10:22863028_ | C | CAAA | 0.9453 | 0.0000 | 1.0000 | 1.0000 |
| | A_C | | | CAAA_C | | | | | | |
| 10 | 10_22863425_A_G | 22863425 | 22574496 | rs12260388 | G | A | 0.9613 | 0.0256 | 1.0259 | 1.0505 |
| 10 | 10_22915624_T_C | 22915624 | 22626695 | rs1359642 | C | T | 0.1594 | −0.0062 | 0.9938 | 0.9980 |
| 10 | 10_23336057_T_C | 23336057 | 23047128 | rs10764375 | C | T | 0.9664 | 0.0405 | 1.0413 | 1.0815 |
| 10 | 10_23728182_T_C | 23728182 | 23439253 | rs7093562 | C | T | 0.0114 | 0.0132 | 1.0133 | 1.0003 |
| 10 | 10_23947566_A_G | 23947566 | 23658637 | rs2150798 | G | A | 0.0126 | 0.0101 | 1.0102 | 1.0003 |
| 10 | 10_26164083_A_G | 26164083 | 25875154 | rs2367985 | G | A | 0.5630 | 0.0071 | 1.0071 | 1.0080 |
| 10 | 10_26687402_C_T | 26687402 | 26398473 | rs17528774 | T | C | 0.6450 | 0.0101 | 1.0102 | 1.0131 |
| 10 | 10_27479070_C_T | 27479070 | 27190141 | rs788211 | T | C | 0.1911 | −0.0078 | 0.9922 | 0.9970 |
| 10 | 10_28119997_G_A | 28119997 | 27831068 | rs61843170 | A | G | 0.0412 | −0.0323 | 0.9682 | 0.9974 |
| 10 | 10_31043077_G_A | 31043077 | 30754148 | rs11008223 | A | G | 0.4930 | 0.0088 | 1.0088 | 1.0087 |
| 10 | 10_31057888_C_G | 31057888 | 30768959 | rs11008239 | G | C | 0.4008 | 0.0009 | 1.0009 | 1.0007 |
| 10 | 10_32094576_TCA_ | 32094576 | 31805648 | 10:32094576_ | T | TCA | 0.4725 | −0.0029 | 0.9971 | 0.9973 |
| | T | | | TCA_T | | | | | | |
| 10 | 10_32270666_G_G | 32270666 | 31981738 | rs35103129 | GA | G | 0.4520 | 0.0026 | 1.0026 | 1.0024 |
| | A | | | | | | | | | |
| 10 | 10_32287347_CT_C | 32287347 | 31998419 | 10:32287347_ | C | CT | 0.7028 | 0.0030 | 1.0030 | 1.0042 |
| | | | | CT_C | | | | | | |
| 10 | 10_32678141_C_T | 32678141 | 32389213 | rs7070638 | T | C | 0.2348 | 0.0052 | 1.0052 | 1.0025 |
| 10 | 10_32908838_CTTT | 32908838 | 32619910 | 10:32908838_ | C | CTTT | 0.8629 | 0.0110 | 1.0111 | 1.0192 |
| | TTTTTTTT_C | | | CTTTTTTTTTTT | | TTTT | | | | |
| | | | | _C | | TTTT | | | | |
| 10 | 10_33089200_A_T | 33089200 | 32800272 | rs2095555 | T | A | 0.1626 | −0.0001 | 0.9999 | 1.0000 |
| 10 | 10_33724400_A_C | 33724400 | 33435472 | rs60101430 | C | A | 0.0153 | 0.0026 | 1.0026 | 1.0001 |
| 10 | 10_33764406_G_A | 33764406 | 33475478 | rs12247780 | A | G | 0.0685 | 0.0087 | 1.0087 | 1.0012 |
| 10 | 10_35214293_A_G | 35214293 | 34925365 | rs10827464 | G | A | 0.2665 | 0.0089 | 1.0089 | 1.0048 |
| 10 | 10_37390610_C_A | 37390610 | 37101682 | rs1200921 | A | C | 0.4628 | −0.0043 | 0.9957 | 0.9960 |
| 10 | 10_37661344_T_A | 37661344 | 37372416 | rs1938420 | A | T | 0.6124 | −0.0013 | 0.9987 | 0.9984 |
| 10 | 10_38530780_T_TA | 38530780 | 38241852 | rs199668839 | TA | T | 0.0633 | 0.0004 | 1.0004 | 1.0001 |
| 10 | 10_38634076_T_TA | 38634076 | 38345148 | rs369577778 | TA | T | 0.4240 | 0.0146 | 1.0147 | 1.0125 |
| 10 | 10_39100944_C_T | 39100944 | 38807813 | rs201983031 | T | C | 0.0772 | 0.0171 | 1.0172 | 1.0027 |
| 10 | 10_43073537_GAC | 43073537 | 42578089 | 10:43073537_ | G | GAC | 0.7593 | 0.0068 | 1.0068 | 1.0104 |
| | _G | | | GAC_G | | | | | | |
| 10 | 10_44285159_T_C | 44285159 | 43789711 | rs1998748 | C | T | 0.0953 | −0.0076 | 0.9924 | 0.9986 |
| 10 | 10_45524771_T_C | 45524771 | 45029323 | rs10900158 | C | T | 0.7143 | −0.0113 | 0.9888 | 0.9840 |
| 10 | 10_45545941_C_G | 45545941 | 45050493 | rs2311846 | G | C | 0.7628 | −0.0067 | 0.9933 | 0.9898 |
| 10 | 10_45642534_G_A | 45642534 | 45147086 | rs1339746 | A | G | 0.9356 | −0.0137 | 0.9864 | 0.9747 |
| 10 | 10_45912771_T_G | 45912771 | 45417323 | rs11239524 | G | T | 0.1541 | 0.0050 | 1.0050 | 1.0015 |
| 10 | 10_49594141_C_A | 49594141 | 48386098 | rs56278337 | A | C | 0.1065 | 0.0017 | 1.0017 | 1.0004 |
| 10 | 10_49717579_C_T | 49717579 | 48509536 | rs72785120 | T | C | 0.1533 | 0.0118 | 1.0119 | 1.0036 |
| 10 | 10_50450147_T_G | 50450147 | 49242102 | rs143362844 | G | T | 0.0112 | 0.0529 | 1.0543 | 1.0012 |
| 10 | 10_51602606_C_A | 51602606 | 45993229 | rs75914490 | A | C | 0.2758 | −0.0098 | 0.9902 | 0.9946 |
| 10 | 10_52696552_T_A | 52696552 | 50936792 | rs74572072 | A | T | 0.0181 | −0.0358 | 0.9648 | 0.9987 |
| 10 | 10_53676045_T_C | 53676045 | 51916285 | rs4935290 | C | T | 0.6715 | −0.0076 | 0.9924 | 0.9899 |
| 10 | 10_53692969_C_T | 53692969 | 51933209 | rs7920008 | T | C | 0.4080 | −0.0001 | 0.9999 | 0.9999 |
| 10 | 10_53773995_G_A | 53773995 | 52014235 | rs4495849 | A | G | 0.1970 | −0.0068 | 0.9932 | 0.9973 |
| 10 | 10_55033677_G_T | 55033677 | 53273917 | rs142145443 | T | G | 0.0156 | −0.0147 | 0.9854 | 0.9995 |
| 10 | 10_56741909_G_T | 56741909 | 54982149 | rs2222872 | T | G | 0.4688 | 0.0000 | 1.0000 | 1.0000 |
| 10 | 10_59111940_T_G | 59111940 | 57352180 | rs1904757 | G | T | 0.1653 | −0.0031 | 0.9969 | 0.9990 |
| 10 | 10_61394137_G_A | 61394137 | 59634379 | rs11599171 | A | G | 0.2403 | 0.0091 | 1.0091 | 1.0044 |
| 10 | 10_61399990_T_C | 61399990 | 59640232 | rs2893807 | C | T | 0.2745 | 0.0000 | 1.0000 | 1.0000 |
| 10 | 10_62099814_C_T | 62099814 | 60340056 | rs10821732 | T | C | 0.0129 | −0.0209 | 0.9793 | 0.9995 |
| 10 | 10_63192414_TA_ | 63192414 | 61432656 | | AA | TA | 0.7840 | 0.0094 | 1.0094 | 1.0149 |
| | AA | | | | | | | | | |
| 10 | 10_63417282_C_T | 63417282 | 61657524 | rs55645517 | T | C | 0.0788 | −0.0061 | 0.9939 | 0.9990 |
| 10 | 10_63425659_AAT | 63425659 | 61665901 | 10:63425659_ | A | AAT | 0.4267 | 0.0049 | 1.0049 | 1.0042 |
| | _A | | | AAT_A | | | | | | |
| 10 | 10_63476983_T_C | 63476983 | 61717225 | rs17281372 | C | T | 0.4134 | 0.0000 | 1.0000 | 1.0000 |
| 10 | 10_63617615_A_G | 63617615 | 61857856 | rs74847539 | G | A | 0.0431 | 0.0035 | 1.0035 | 1.0003 |
| 10 | 10_63871993_T_A | 63871993 | 62112234 | rs57591366 | A | T | 0.3785 | 0.0000 | 1.0000 | 1.0000 |
| 10 | 10_64219094_T_C | 64219094 | 62459335 | rs78021475 | C | T | 0.0124 | 0.0102 | 1.0103 | 1.0003 |
| 10 | 10_64257828_T_C | 64257828 | 62498069 | rs10509168 | C | T | 0.5227 | 0.0174 | 1.0176 | 1.0184 |
| 10 | 10_64263402_C_G | 64263402 | 62503643 | rs4746409 | G | C | 0.5052 | −0.0055 | 0.9945 | 0.9945 |
| 10 | 10_64268764_T_C | 64268764 | 62509005 | rs10995182 | C | T | 0.1335 | −0.0114 | 0.9887 | 0.9970 |
| 10 | 10_64293571_AAA | 64293571 | 62533812 | rs764349609 | A | AAAG | 0.3392 | 0.0088 | 1.0088 | 1.0060 |
| | GG_A | | | | | G | | | | |
| 10 | 10_64299890_A_G | 64299890 | 62540131 | rs10995201 | G | A | 0.1603 | −0.0819 | 0.9214 | 0.9750 |
| 10 | 10_64309151_C_C | 64309151 | 62549392 | rs10638039 | CAT | C | 0.3933 | 0.0043 | 1.0043 | 1.0034 |
| | AT | | | | | | | | | |
| 10 | 10_64414265_C_T | 64414265 | 62654505 | rs34632941 | T | C | 0.0752 | −0.0031 | 0.9969 | 0.9995 |
| 10 | 10_64440842_T_C | 64440842 | 62681082 | rs16917616 | C | T | 0.0105 | 0.0000 | 1.0000 | 1.0000 |
| 10 | 10_64562524_G_A | 64562524 | 62802764 | rs7905654 | A | G | 0.1184 | −0.0036 | 0.9964 | 0.9991 |
| 10 | 10_64578483_C_T | 64578483 | 62818723 | rs61865884 | T | C | 0.0700 | −0.0164 | 0.9837 | 0.9977 |
| 10 | 10_64582482_T_C | 64582482 | 62822722 | rs61865900 | C | T | 0.0617 | −0.0107 | 0.9894 | 0.9987 |
| 10 | 10_64611042_A_G | 64611042 | 62851282 | rs117807754 | G | A | 0.0246 | 0.0171 | 1.0172 | 1.0008 |
| 10 | 10_64763893_G_A | 64763893 | 63004133 | rs11595405 | A | G | 0.4803 | 0.0016 | 1.0016 | 1.0015 |
| 10 | 10_64802682_GA_ | 64802682 | 63042922 | rs35598651 | G | GA | 0.3303 | 0.0076 | 1.0076 | 1.0050 |
| | G | | | | | | | | | |
| 10 | 10_64817375_C_T | 64817375 | 63057615 | rs34942900 | T | C | 0.0374 | 0.0000 | 1.0000 | 1.0000 |
| 10 | 10_64819996_G_T | 64819996 | 63060236 | rs6479868 | T | G | 0.1958 | 0.0090 | 1.0090 | 1.0035 |
| 10 | 10_65224458_CA_ | 65224458 | 63464698 | 10:65224458_ | C | CA | 0.5455 | −0.0014 | 0.9986 | 0.9985 |
| | C | | | CA_C | | | | | | |
| 10 | 10_67686579_T_C | 67686579 | 65926821 | rs12251157 | C | T | 0.0389 | −0.0081 | 0.9919 | 0.9994 |
| 10 | 10_68380961_A_G | 68380961 | 66621203 | rs144769797 | G | A | 0.0223 | −0.0317 | 0.9688 | 0.9986 |
| 10 | 10_71078937_A_G | 71078937 | 69319181 | rs75634211 | G | A | 0.0280 | −0.0018 | 0.9982 | 0.9999 |
| 10 | 10_71147630_A_A | 71147630 | 69387874 | rs11424399 | AT | A | 0.4766 | 0.0022 | 1.0022 | 1.0021 |
| | T | | | | | | | | | |
| 10 | 10_71152091_T_C | 71152091 | 69392335 | rs2278745 | C | T | 0.5242 | 0.0030 | 1.0030 | 1.0032 |
| 10 | 10_71333897_C_T | 71333897 | 69574141 | rs12785008 | T | C | 0.1740 | −0.0026 | 0.9974 | 0.9991 |
| 10 | 10_71335574_C_T | 71335574 | 69575818 | rs111833376 | T | C | 0.3179 | −0.0164 | 0.9837 | 0.9897 |
| 10 | 10_72765137_G_A | 72765137 | 71005380 | rs75693169 | A | G | 0.0495 | 0.0280 | 1.0284 | 1.0028 |
| 10 | 10_73649779_C_T | 73649779 | 71890021 | rs7915996 | T | C | 0.1882 | 0.0058 | 1.0058 | 1.0022 |
| 10 | 10_75383954_G_T | 75383954 | 73624196 | rs556862051 | T | G | 0.0410 | −0.0120 | 0.9881 | 0.9990 |
| 10 | 10_75400405_A_T | 75400405 | 73640647 | rs11000726 | T | A | 0.1177 | −0.0126 | 0.9875 | 0.9971 |
| 10 | 10_80290002_G_A | 80290002 | 78530245 | rs117315821 | A | G | 0.0261 | −0.0503 | 0.9509 | 0.9974 |
| 10 | 10_80322047_ACA | 80322047 | 78562290 | 10:80322047_ | A | ACAG | 0.0431 | −0.0037 | 0.9963 | 0.9997 |
| | GTGCTGTCT_A | | | ACAGTGCTGT | | TGCT | | | | |
| | | | | CT_A | | GTCT | | | | |
| 10 | 10_80830292_T_C | 80830292 | 79070535 | rs2485698 | C | T | 0.9249 | 0.0000 | 1.0000 | 1.0000 |
| 10 | 10_80833595_C_C | 80833595 | 79073838 | rs34518733 | CAG | C | 0.4438 | 0.0000 | 1.0000 | 1.0000 |
| | AG | | | | | | | | | |
| 10 | 10_80835868_G_A | 80835868 | 79076111 | rs10824713 | A | G | 0.5805 | 0.0000 | 1.0000 | 1.0000 |
| 10 | 10_80836590_C_T | 80836590 | 79076833 | rs1658329 | T | C | 0.6233 | −0.0098 | 0.9902 | 0.9879 |
| 10 | 10_80842416_T_C | 80842416 | 79082659 | rs704009 | C | T | 0.7779 | 0.0000 | 1.0000 | 1.0000 |
| 10 | 10_80847150_C_T | 80847150 | 79087393 | rs1250002 | T | C | 0.1828 | −0.0037 | 0.9963 | 0.9987 |
| 10 | 10_80851257_G_T | 80851257 | 79091500 | rs719338 | T | G | 0.6172 | −0.0305 | 0.9700 | 0.9633 |
| 10 | 10_80852892_AAC | 80852892 | 79093135 | 10:80852892_ | A | AACA | 0.4946 | −0.0145 | 0.9856 | 0.9858 |
| | AC_A | | | AACAC_A | | C | | | | |
| 10 | 10_80855650_G_A | 80855650 | 79095893 | rs11002811 | A | G | 0.1567 | 0.0063 | 1.0063 | 1.0020 |
| 10 | 10_80870324_G_C | 80870324 | 79110567 | rs7914988 | C | G | 0.5861 | 0.0039 | 1.0039 | 1.0046 |
| 10 | 10_80882589_A_G | 80882589 | 79122832 | rs1658322 | G | A | 0.4397 | 0.0008 | 1.0008 | 1.0007 |
| 10 | 10_80883311_T_C | 80883311 | 79123554 | rs1866157 | C | T | 0.1927 | 0.0124 | 1.0125 | 1.0048 |
| 10 | 10_80886726_A_G | 80886726 | 79126969 | rs4980029 | G | A | 0.1631 | 0.0303 | 1.0308 | 1.0101 |
| 10 | 10_80926036_T_C | 80926036 | 79166279 | rs703994 | C | T | 0.5562 | 0.0020 | 1.0020 | 1.0022 |
| 10 | 10_84397685_AT_ | 84397685 | 82637929 | rs201016760 | A | AT | 0.6268 | −0.0003 | 0.9997 | 0.9996 |
| | A | | | | | | | | | |
| 10 | 10_85694849_G_C | 85694849 | 83935093 | rs7099146 | C | G | 0.1752 | 0.0089 | 1.0089 | 1.0031 |
| 10 | 10_88440446_CA_ | 88440446 | 86680689 | rs753019787 | C | CA | 0.2154 | 0.0070 | 1.0070 | 1.0030 |
| | C | | | | | | | | | |
| 10 | 10_88832746_C_T | 88832746 | 87072989 | rs9421579 | T | C | 0.0908 | 0.0021 | 1.0021 | 1.0004 |
| 10 | 10_90112623_C_G | 90112623 | 88352866 | rs1086833 | G | C | 0.2542 | −0.0007 | 0.9993 | 0.9996 |
| 10 | 10_90151393_T_A | 90151393 | 88391636 | rs1774973 | A | T | 0.4614 | −0.0009 | 0.9991 | 0.9992 |
| 10 | 10_90198801_A_G | 90198801 | 88439044 | rs17426488 | G | A | 0.2552 | −0.0086 | 0.9914 | 0.9956 |
| 10 | 10_91784322_C_T | 91784322 | 90024565 | rs11185975 | T | C | 0.2349 | 0.0012 | 1.0012 | 1.0006 |
| 10 | 10_91824299_A_C | 91824299 | 90064542 | rs7900020 | C | A | 0.2291 | 0.0054 | 1.0054 | 1.0025 |
| 10 | 10_92537170_G_A | 92537170 | 90777413 | rs12416468 | A | G | 0.0655 | −0.0138 | 0.9863 | 0.9982 |
| 10 | 10_92559908_G_T | 92559908 | 90800151 | rs7916403 | T | G | 0.4358 | −0.0035 | 0.9965 | 0.9970 |
| 10 | 10_92604139_G_A | 92604139 | 90844382 | rs7084468 | A | G | 0.1668 | −0.0036 | 0.9964 | 0.9988 |
| 10 | 10_92803107_G_A | 92803107 | 91043350 | rs117414562 | A | G | 0.0463 | 0.0035 | 1.0035 | 1.0003 |
| 10 | 10_93569805_A_G | 93569805 | 91810048 | rs79611925 | G | A | 0.0273 | −0.0083 | 0.9917 | 0.9995 |
| 10 | 10_93932286_A_G | 93932286 | 92172529 | rs72807246 | G | A | 0.1415 | −0.0057 | 0.9943 | 0.9984 |
| 10 | 10_95292187_CAA | 95292187 | 93532430 | 10:95292187_ | C | CAA | 0.8234 | −0.0187 | 0.9815 | 0.9697 |
| | _C | | | CAA_C | | | | | | |
| 10 | 10_95725628_C_T | 95725628 | 93965871 | rs10882371 | T | C | 0.7145 | 0.0065 | 1.0065 | 1.0093 |
| 10 | 10_97810637_T_C | 97810637 | 96050880 | rs880518 | C | T | 0.0138 | 0.0000 | 1.0000 | 1.0000 |
| 10 | 10_101130302_A_T | 101130302 | 99370545 | rs1892511 | T | A | 0.8815 | 0.0101 | 1.0102 | 1.0180 |
| 10 | 10_102936499_C_ | 102936499 | 101176742 | rs10883594 | G | C | 0.1116 | −0.0139 | 0.9862 | 0.9969 |
| | G | | | | | | | | | |
| 10 | 10_104168611_G_ | 104168611 | 102408854 | rs148440547 | T | G | 0.0675 | 0.0052 | 1.0052 | 1.0007 |
| | T | | | | | | | | | |
| 10 | 10_106354417_C_T | 106354417 | 104594659 | rs11192129 | T | C | 0.1396 | 0.0024 | 1.0024 | 1.0007 |
| 10 | 10_106385271_G_ | 106385271 | 104625513 | rs1577222 | T | G | 0.6041 | −0.0011 | 0.9989 | 0.9987 |
| | T | | | | | | | | | |
| 10 | 10_107275646_T_T | 107275646 | 105515888 | rs201520282 | TAC | T | 0.1334 | −0.0116 | 0.9885 | 0.9969 |
| | AC | | | | | | | | | |
| 10 | 10_110988757_T_C | 110988757 | 109228999 | rs10884721 | C | T | 0.5959 | 0.0083 | 1.0083 | 1.0100 |
| 10 | 10_111348202_G_ | 111348202 | 109588444 | rs1936146 | A | G | 0.4668 | 0.0088 | 1.0088 | 1.0083 |
| | A | | | | | | | | | |
| 10 | 10_111967424_G_ | 111967424 | 110207666 | rs76313351 | C | G | 0.0158 | −0.0161 | 0.9840 | 0.9995 |
| | C | | | | | | | | | |
| 10 | 10_112197458_C_T | 112197458 | 110437700 | rs1981112 | T | C | 0.2537 | 0.0006 | 1.0006 | 1.0003 |
| 10 | 10 112332822_TTG | 112332822 | 110573064 | 10:112332822 | T | TTG | 0.0110 | 0.0000 | 1.0000 | 1.0000 |
| | _T | | | _TTG_T | | | | | | |
| 10 | 10_114551367_T_C | 114551367 | 112791608 | rs141549560 | C | T | 0.0297 | −0.0378 | 0.9629 | 0.9978 |
| 10 | 10_114722935_A_ | 114722935 | 112963176 | rs61875105 | G | A | 0.0887 | 0.0000 | 1.0000 | 1.0000 |
| | G | | | | | | | | | |
| 10 | 10_114732906_A_C | 114732906 | 112973147 | rs7901275 | C | A | 0.4495 | 0.0018 | 1.0018 | 1.0016 |
| 10 | 10_114773887_T_C | 114773887 | 113014128 | rs55938323 | C | T | 0.0111 | 0.0006 | 1.0006 | 1.0000 |
| 10 | 10_114777670_C_T | 114777670 | 113017911 | rs10885405 | T | C | 0.4631 | 0.0193 | 1.0195 | 1.0181 |
| 10 | 10_114793572_T_C | 114793572 | 113033813 | rs116369954 | C | T | 0.0279 | 0.0059 | 1.0059 | 1.0003 |
| 10 | 10_114887002_GT | 114887002 | 113127243 | 10:114887002 | G | GT | 0.6110 | 0.0098 | 1.0098 | 1.0121 |
| | _G | | | _GT_G | | | | | | |
| 10 | 10 115127454_C_C | 115127454 | 113367695 | | CAA | C | 0.7057 | −0.0081 | 0.9919 | 0.9886 |
| | AA | | | | | | | | | |
| 10 | 10_115128491_T_C | 115128491 | 113368732 | rs12250948 | C | T | 0.7846 | −0.0277 | 0.9727 | 0.9576 |
| 10 | 10_115465925_G_ | 115465925 | 113706166 | rs34293126 | T | G | 0.2293 | 0.0104 | 1.0105 | 1.0048 |
| | T | | | | | | | | | |
| 10 | 10_115543224_T_C | 115543224 | 113783465 | rs12354481 | C | T | 0.2011 | 0.0000 | 1.0000 | 1.0000 |
| 10 | 10_115720030_G_ | 115720030 | 113960271 | rs58338978 | A | G | 0.0661 | 0.0002 | 1.0002 | 1.0000 |
| | A | | | | | | | | | |
| 10 | 10_119249094_G_ | 119249094 | 117489583 | rs143855912 | GGTT | G | 0.1375 | −0.0045 | 0.9955 | 0.9988 |
| | GGTT | | | | | | | | | |
| 10 | 10_119262365_A_ | 119262365 | 117502854 | rs2901157 | G | A | 0.1147 | −0.0078 | 0.9922 | 0.9982 |
| | G | | | | | | | | | |
| 10 | 10_119679243_T_C | 119679243 | 117919732 | rs12242896 | C | T | 0.0677 | −0.0141 | 0.9860 | 0.9981 |
| 10 | 10_120541197_C_T | 120541197 | 118781685 | rs962890 | T | C | 0.7345 | −0.0012 | 0.9988 | 0.9982 |
| 10 | 10_120822033_T_ | 120822033 | 119062521 | rs2152240 | G | T | 0.4115 | −0.0031 | 0.9969 | 0.9975 |
| | G | | | | | | | | | |
| 10 | 10_120851811_T_C | 120851811 | 119092299 | rs2420491 | C | T | 0.5933 | 0.0043 | 1.0043 | 1.0051 |
| 10 | 10_121381615_A_ | 121381615 | 119622103 | rs196310 | G | A | 0.4770 | 0.0039 | 1.0039 | 1.0037 |
| | G | | | | | | | | | |
| 10 | 10_121405714_G_ | 121405714 | 119646202 | rs7083324 | A | G | 0.2455 | 0.0043 | 1.0043 | 1.0021 |
| | A | | | | | | | | | |
| 10 | 10_122230768_A_ | 122230768 | 120471256 | rs142147598 | G | A | 0.0278 | −0.0408 | 0.9600 | 0.9978 |
| | G | | | | | | | | | |
| 10 | 10_122752466_A_T | 122752466 | 120992953 | rs12359502 | T | A | 0.3175 | −0.0022 | 0.9978 | 0.9986 |
| 10 | 10_122755774_G_ | 122755774 | 120996261 | rs11199696 | A | G | 0.2253 | 0.0024 | 1.0024 | 1.0011 |
| | A | | | | | | | | | |
| 10 | 10_122802462_A_C | 122802462 | 121042949 | rs748476 | C | A | 0.1778 | 0.0149 | 1.0150 | 1.0053 |
| 10 | 10_122837858_T_C | 122837858 | 121078345 | rs732640 | C | T | 0.2555 | −0.0109 | 0.9892 | 0.9945 |
| 10 | 10_123094959_GA | 123094959 | 121335445 | 10:123094959 | G | GAA | 0.2921 | 0.0000 | 1.0000 | 1.0000 |
| | A_G | | | GAA_G | | | | | | |
| 10 | 10 123095209 G_ | 123095209 | 121335695 | rs9421410 | A | G | 0.3269 | −0.0273 | 0.9731 | 0.9825 |
| | A | | | | | | | | | |
| 10 | 10_123153472_A_ | 123153472 | 121393958 | rs2420928 | G | A | 0.4207 | 0.0084 | 1.0084 | 1.0071 |
| | G | | | | | | | | | |
| 10 | 10_123155416_C_T | 123155416 | 121395902 | rs4751835 | T | C | 0.3647 | 0.0016 | 1.0016 | 1.0012 |
| 10 | 10_123240645_C_T | 123240645 | 121481131 | rs3135821 | T | C | 0.0999 | −0.0084 | 0.9916 | 0.9983 |
| 10 | 10_123320793_C_T | 123320793 | 121561279 | rs145549294 | T | C | 0.0142 | 0.0000 | 1.0000 | 1.0000 |
| 10 | 10_123331690_A_T | 123331690 | 121572176 | rs4752569 | T | A | 0.4860 | 0.0000 | 1.0000 | 1.0000 |
| 10 | 10_123340311_C_T | 123340311 | 121580797 | rs2981578 | T | C | 0.5295 | −0.0223 | 0.9779 | 0.9768 |
| 10 | 10_123340431_GC | 123340431 | 121580917 | 10:123340431 | G | GC | 0.5963 | −0.1732 | 0.8410 | 0.8193 |
| | G | | | _GC_G | | | | | | |
| 10 | 10_123343909_T_ | 123343909 | 121584395 | rs1696840 | G | T | 0.3860 | 0.0000 | 1.0000 | 1.0000 |
| | G | | | | | | | | | |
| 10 | 10_123346116_G_ | 123346116 | 121586602 | rs2981575 | A | G | 0.6067 | −0.0137 | 0.9864 | 0.9836 |
| | A | | | | | | | | | |
| 10 | 10_123349324_A_T | 123349324 | 121589810 | rs45631563 | T | A | 0.0484 | −0.0880 | 0.9158 | 0.9919 |
| 10 | 10_123356175_A_T | 123356175 | 121596661 | rs2936871 | T | A | 0.8416 | −0.0065 | 0.9935 | 0.9891 |
| 10 | 10 123484962_G_ | 123484962 | 121725447 | rs12771557 | A | G | 0.3796 | −0.0001 | 0.9999 | 0.9999 |
| | A | | | | | | | | | |
| 10 | 10_123788291_G_ | 123788291 | 122028776 | rs61679579 | C | G | 0.5760 | 0.0000 | 1.0000 | 1.0000 |
| | C | | | | | | | | | |
| 10 | 10_123800974_C_T | 123800974 | 122041459 | rs2459078 | T | C | 0.6139 | 0.0000 | 1.0000 | 1.0000 |
| 10 | 10_127258691_G_ | 127258691 | 125570122 | rs7084502 | A | G | 0.4132 | 0.0077 | 1.0077 | 1.0064 |
| | A | | | | | | | | | |
| 10 | 10_127981161_T_C | 127981161 | 126292592 | rs4962336 | C | T | 0.3085 | 0.0092 | 1.0092 | 1.0057 |
| 10 | 10 129233816_T_ | 129233816 | 127435552 | rs11816982 | G | T | 0.1199 | −0.0048 | 0.9952 | 0.9989 |
| | G | | | | | | | | | |
| 10 | 10_129248015_G_ | 129248015 | 127449751 | rs55798271 | C | G | 0.1009 | 0.0027 | 1.0027 | 1.0005 |
| | C | | | | | | | | | |
| 10 | 10_133833155_T_ | 133833155 | 132019651 | rs117413668 | G | T | 0.0967 | 0.0044 | 1.0044 | 1.0009 |
| | G | | | | | | | | | |
| 10 | 10_133947465_C_A | 133947465 | 132133961 | rs78501832 | A | C | 0.0719 | −0.0089 | 0.9911 | 0.9987 |
| 10 | 10_134561926_C_T | 134561926 | 132748422 | rs11146497 | T | C | 0.0725 | −0.0103 | 0.9898 | 0.9985 |
| 11 | 11_322722_C_T | 322722 | 322722 | rs10794307 | T | C | 0.3530 | −0.0008 | 0.9992 | 0.9994 |
| 11 | 11_418735_G_A | 418735 | 418735 | rs12792208 | A | G | 0.0202 | 0.0039 | 1.0039 | 1.0002 |
| 11 | 11_428489_T_C | 428489 | 428489 | rs10794324 | C | T | 0.7746 | −0.0174 | 0.9828 | 0.9735 |
| 11 | 11_433617_T_C | 433617 | 433617 | rs7394715 | C | T | 0.7969 | −0.0025 | 0.9975 | 0.9960 |
| 11 | 11_445331_C_T | 445331 | 445331 | rs71487288 | T | C | 0.1448 | −0.0103 | 0.9898 | 0.9970 |
| 11 | 11_701616_A_G | 701616 | 701616 | rs7112603 | G | A | 0.9095 | 0.0042 | 1.0042 | 1.0077 |
| 11 | 11_803017_A_G | 803017 | 803017 | rs6597981 | G | A | 0.5167 | 0.0192 | 1.0194 | 1.0201 |
| 11 | 11_835407_C_T | 835407 | 835407 | rs7126859 | T | C | 0.5106 | 0.0074 | 1.0074 | 1.0076 |
| 11 | 11_1091080_G_A | 1091080 | 1093142 | rs41414549 | A | G | 0.0359 | 0.0047 | 1.0047 | 1.0003 |
| 11 | 11_1876228_C_T | 1876228 | 1854998 | rs11602798 | T | C | 0.2643 | 0.0000 | 1.0000 | 1.0000 |
| 11 | 11_1891284_T_A | 1891284 | 1870054 | rs688601 | A | T | 0.5486 | −0.0215 | 0.9787 | 0.9768 |
| 11 | 11_1895708_C_A | 1895708 | 1874478 | rs4980386 | A | C | 0.3924 | −0.0031 | 0.9969 | 0.9976 |
| 11 | 11_1900765_C_T | 1900765 | 1879535 | rs11041615 | T | C | 0.1752 | −0.0072 | 0.9928 | 0.9975 |
| 11 | 11_1902097_C_T | 1902097 | 1880867 | rs4980383 | T | C | 0.4404 | 0.0077 | 1.0077 | 1.0068 |
| 11 | 11_1902475_G_A | 1902475 | 1881245 | rs620315 | A | G | 0.3842 | −0.0160 | 0.9841 | 0.9878 |
| 11 | 11_1905292_T_C | 1905292 | 1884062 | rs661348 | C | T | 0.4361 | −0.0055 | 0.9945 | 0.9952 |
| 11 | 11_1948186_T_C | 1948186 | 1926956 | rs7125631 | C | T | 0.3530 | 0.0056 | 1.0056 | 1.0040 |
| 11 | 11_1951960_G_C | 1951960 | 1930730 | rs10466507 | C | G | 0.1542 | 0.0000 | 1.0000 | 1.0000 |
| 11 | 11_1975106_T_C | 1975106 | 1953876 | rs757409 | C | T | 0.2939 | 0.0000 | 1.0000 | 1.0000 |
| 11 | 11_1979118_C_G | 1979118 | 1957888 | rs217213 | G | C | 0.4215 | −0.0031 | 0.9969 | 0.9974 |
| 11 | 11_1979223_G_C | 1979223 | 1957993 | rs2063550 | C | G | 0.5189 | 0.0031 | 1.0031 | 1.0032 |
| 11 | 11_1997572_− | 1997572 | 1976342 | rs112088979 | GTCC | G | 0.4328 | 0.0008 | 1.0008 | 1.0007 |
| | GTCC | | | | | | | | | |
| 11 | 11_2016908_G_A | 2016908 | 1995678 | rs217727 | A | G | 0.1926 | −0.0041 | 0.9959 | 0.9984 |
| 11 | 11_2024544_G_A | 2024544 | 2003314 | rs4930103 | A | G | 0.5249 | 0.0059 | 1.0059 | 1.0062 |
| 11 | 11_2223850_G_C | 2223850 | 2202620 | rs10840550 | C | G | 0.5600 | 0.0043 | 1.0043 | 1.0048 |
| 11 | 11_2332694_A_G | 2332694 | 2311464 | rs80120162 | G | A | 0.0477 | 0.0185 | 1.0187 | 1.0018 |
| 11 | 11_2354538_T_C | 2354538 | 2333308 | rs567031996 | C | T | 0.0410 | 0.0004 | 1.0004 | 1.0000 |
| 11 | 11_2390064_G_A | 2390064 | 2368834 | rs77369403 | A | G | 0.0401 | 0.0028 | 1.0028 | 1.0002 |
| 11 | 11_2830946_CG_C | 2830946 | 2809716 | rs775385647 | C | CG | 0.0920 | −0.0080 | 0.9920 | 0.9985 |
| 11 | 11_2894562_C_A | 2894562 | 2873332 | rs111875758 | A | C | 0.0222 | −0.0256 | 0.9747 | 0.9989 |
| 11 | 11_3215893_G_T | 3215893 | 3194663 | rs72848229 | T | G | 0.0606 | 0.0249 | 1.0252 | 1.0031 |
| 11 | 11_3223621_G_A | 3223621 | 3202391 | rs72848239 | A | G | 0.0383 | 0.0072 | 1.0072 | 1.0006 |
| 11 | 11_5768236_C_G | 5768236 | 5747006 | rs36173363 | G | C | 0.0977 | 0.0009 | 1.0009 | 1.0002 |
| 11 | 11_6255984_C_T | 6255984 | 6234754 | rs12274456 | T | C | 0.2463 | 0.0088 | 1.0088 | 1.0044 |
| 11 | 11_9726369_T_C | 9726369 | 9704822 | rs34043654 | C | T | 0.3496 | −0.0083 | 0.9917 | 0.9942 |
| 11 | 11_9936951_G_A | 9936951 | 9915404 | rs4910077 | A | G | 0.2951 | 0.0038 | 1.0038 | 1.0022 |
| 11 | 11_10147968_T_C | 10147968 | 10126421 | rs12363292 | C | T | 0.2240 | −0.0131 | 0.9870 | 0.9942 |
| 11 | 11_10472239_A_G | 10472239 | 10450692 | rs61891367 | G | A | 0.1757 | −0.0132 | 0.9869 | 0.9954 |
| 11 | 11_10540013_CTG | 10540013 | 10518466 | 11:10540013_ | C | CTGA | 0.3415 | −0.0090 | 0.9910 | 0.9939 |
| | AA_C | | | CTGAA_C | | A | | | | |
| 11 | 11_11498224_G_C | 11498224 | 11476677 | rs34203081 | C | G | 0.0678 | 0.0062 | 1.0062 | 1.0008 |
| 11 | 11_11596321_- | 11596321 | 11574774 | rs11386655 | CT | C | 0.6763 | 0.0002 | 1.0002 | 1.0003 |
| | _CT | | | | | | | | | |
| 11 | 11_11671547_ | 11671547 | 11650000 | 11:11671547_ | T | TA | 0.0544 | −0.0160 | 0.9841 | 0.9983 |
| | TA_T | | | TA_T | | | | | | |
| 11 | 11_14014644_ | 14014644 | 13993097 | rs781998923 | T | TTA | 0.1185 | 0.0106 | 1.0107 | 1.0025 |
| | TTA_T | | | | | | | | | |
| 11 | 11_15333431_C_T | 15333431 | 15311885 | rs2350987 | T | C | 0.4678 | −0.0092 | 0.9908 | 0.9915 |
| 11 | 11_16593142_C_A | 16593142 | 16571595 | rs72859467 | A | C | 0.1514 | 0.0003 | 1.0003 | 1.0001 |
| 11 | 11_16644500_G_A | 16644500 | 16622953 | rs72861246 | A | G | 0.0823 | 0.0046 | 1.0046 | 1.0008 |
| 11 | 11_17455371_G_A | 17455371 | 17433824 | rs4148617 | A | G | 0.2455 | −0.0079 | 0.9921 | 0.9961 |
| 11 | 11_17462858_T_C | 17462858 | 17441311 | rs873314 | C | T | 0.2083 | −0.0062 | 0.9938 | 0.9974 |
| 11 | 11_18403867_A_G | 18403867 | 18382320 | rs10766474 | G | A | 0.5474 | 0.0010 | 1.0010 | 1.0011 |
| 11 | 11_18664241_T_G | 18664241 | 18642694 | rs10832963 | G | T | 0.7293 | 0.0203 | 1.0205 | 1.0301 |
| 11 | 11_18666897_C_T | 18666897 | 18645350 | rs72882183 | T | C | 0.0647 | −0.0063 | 0.9937 | 0.9992 |
| 11 | 11_18687025_G_A | 18687025 | 18665478 | rs12787397 | A | G | 0.2940 | 0.0024 | 1.0024 | 1.0014 |
| 11 | 11_22610647_T_C | 22610647 | 22589101 | rs11026677 | C | T | 0.1902 | −0.0001 | 0.9999 | 1.0000 |
| 11 | 11_24366115_T_C | 24366115 | 24344569 | rs6484042 | C | T | 0.1730 | −0.0036 | 0.9964 | 0.9988 |
| 11 | 11_25390994_A_T | 25390994 | 25369448 | rs7934880 | T | A | 0.1136 | −0.0014 | 0.9986 | 0.9997 |
| 11 | 11_25391785_C_T | 25391785 | 25370239 | rs7935930 | T | C | 0.1165 | −0.0031 | 0.9969 | 0.9993 |
| 11 | 11_25408575_T_C | 25408575 | 25387029 | rs78467396 | C | T | 0.0138 | −0.0218 | 0.9784 | 0.9994 |
| 11 | 11_25518333_T_G | 25518333 | 25496787 | rs79087301 | G | T | 0.1468 | −0.0033 | 0.9967 | 0.9990 |
| 11 | 11_25614733_ATA | 25614733 | 25593187 | rs776163159 | A | ATAT | 0.1625 | −0.0058 | 0.9942 | 0.9981 |
| | TACT_A | | | | | ACT | | | | |
| 11 | 11_25624927 _- | 25624927 | 25603381 | rs374893599 | CTATA | C | 0.1220 | −0.0003 | 0.9997 | 0.9999 |
| | CTATA | | | | | | | | | |
| 11 | 11_27085203_CA_ | 27085203 | 27063656 | 11:27085203_ | C | CA | 0.4195 | 0.0038 | 1.0038 | 1.0032 |
| | C | | | CA_C | | | | | | |
| 11 | 11_27428264_C_A | 27428264 | 27406717 | rs12787344 | A | C | 0.2200 | 0.0000 | 1.0000 | 1.0000 |
| 11 | 11_31589116_G_A | 31589116 | 31567569 | rs80001711 | A | G | 0.0640 | −0.0118 | 0.9883 | 0.9985 |
| 11 | 11_31605896_A_T | 31605896 | 31584349 | rs11031419 | T | A | 0.0341 | −0.0005 | 0.9995 | 1.0000 |
| 11 | 11_34728612_ | 34728612 | 34707065 | 11:34728612_ | T | TC | 0.1918 | 0.0068 | 1.0068 | 1.0026 |
| | TC_T | | | TC_T | | | | | | |
| 11 | 11_35331642_G_A | 35331642 | 35310095 | rs3903687 | A | G | 0.6171 | −0.0018 | 0.9982 | 0.9978 |
| 11 | 11_35487857_T_C | 35487857 | 35466309 | rs11602786 | C | T | 0.1579 | 0.0091 | 1.0091 | 1.0029 |
| 11 | 11_35841576_T_C | 35841576 | 35820026 | rs10501143 | C | T | 0.1913 | −0.0146 | 0.9855 | 0.9945 |
| 11 | 11_35890696_C_T | 35890696 | 35869146 | rs7131104 | T | C | 0.1630 | 0.0000 | 1.0000 | 1.0000 |
| 11 | 11_42841708_C_T | 42841708 | 42820158 | rs10768892 | T | C | 0.5972 | 0.0000 | 1.0000 | 1.0000 |
| 11 | 11_42844441_C_T | 42844441 | 42822891 | rs4472923 | T | C | 0.3279 | −0.0133 | 0.9868 | 0.9914 |
| 11 | 11_44169325 _− | 44169325 | 44147775 | rs374108596 | CTTTT | C | 0.8577 | 0.0113 | 1.0114 | 1.0196 |
| | _CTTTT | | | | | | | | | |
| 11 | 11_44368892_G_A | 44368892 | 44347342 | rs10838267 | A | G | 0.5495 | 0.0105 | 1.0106 | 1.0116 |
| 11 | 11_46318032_C_G | 46318032 | 46296481 | rs77047825 | G | C | 0.0659 | −0.0235 | 0.9768 | 0.9969 |
| 11 | 11_46350213_AG_ | 46350213 | 46328663 | rs773391321 | A | AG | 0.1407 | 0.0077 | 1.0077 | 1.0022 |
| | A | | | | | | | | | |
| 11 | 11_47377283_A_G | 47377283 | 47355732 | rs71475909 | G | A | 0.3615 | 0.0000 | 1.0000 | 1.0000 |
| 11 | 11_47980568_T_G | 47980568 | 47959016 | rs7939345 | G | T | 0.7916 | 0.0017 | 1.0017 | 1.0027 |
| 11 | 11_48113430_A_C | 48113430 | 48091878 | rs12576134 | C | A | 0.1613 | −0.0056 | 0.9944 | 0.9982 |
| 11 | 11_55279127_TTC_ | 55279127 | 55511651 | 11:55279127_ | T | TTC | 0.2956 | −0.0041 | 0.9959 | 0.9976 |
| | T | | | TTC_T | | | | | | |
| 11 | 11_55303837_A_G | 55303837 | 55536361 | rs58194256 | G | A | 0.1183 | −0.0057 | 0.9943 | 0.9987 |
| 11 | 11_61819018_T_G | 61819018 | 62051546 | rs6591691 | G | T | 0.2886 | 0.0000 | 1.0000 | 1.0000 |
| 11 | 11_61880295_C_ | 61880295 | 62112823 | rs11408362 | CT | C | 0.3829 | 0.0042 | 1.0042 | 1.0032 |
| | CT | | | | | | | | | |
| 11 | 11_61895444_A_G | 61895444 | 62127972 | rs79215615 | G | A | 0.3351 | −0.0047 | 0.9953 | 0.9969 |
| 11 | 11_61937658_C_T | 61937658 | 62170186 | rs7127472 | T | C | 0.2100 | 0.0025 | 1.0025 | 1.0011 |
| 11 | 11_61960809_C_A | 61960809 | 62193337 | rs7951859 | A | C | 0.7410 | −0.0054 | 0.9946 | 0.9920 |
| 11 | 11_61974845_G_G | 61974845 | 62207373 | rs536206626 | GA | G | 0.3371 | −0.0018 | 0.9982 | 0.9988 |
| | A | | | | | | | | | |
| 11 | 11_62183438_A_G | 62183438 | 62415966 | rs2509971 | G | A | 0.8543 | 0.0001 | 1.0001 | 1.0002 |
| 11 | 11_62183838_C_T | 62183838 | 62416366 | rs3758962 | T | C | 0.1652 | −0.0086 | 0.9914 | 0.9972 |
| 11 | 11_62187269_G_A | 62187269 | 62419797 | rs12270961 | A | G | 0.1976 | −0.0071 | 0.9929 | 0.9972 |
| 11 | 11_62679450_A_G | 62679450 | 62911978 | rs118112787 | G | A | 0.0310 | 0.0017 | 1.0017 | 1.0001 |
| 11 | 11_62772106_G_A | 62772106 | 63004634 | rs7113486 | A | G | 0.0509 | 0.0249 | 1.0252 | 1.0026 |
| 11 | 11_64618768_T_C | 64618768 | 64851296 | rs10897532 | C | T | 0.7060 | −0.0068 | 0.9932 | 0.9905 |
| 11 | 11_65553492_C_A | 65553492 | 65786021 | rs12287832 | A | C | 0.1867 | 0.0164 | 1.0165 | 1.0062 |
| 11 | 11_65572431_G_A | 65572431 | 65804960 | rs10896047 | A | G | 0.4886 | −0.0220 | 0.9782 | 0.9789 |
| 11 | 11_65601650_G_C | 65601650 | 65834179 | rs548082010 | C | G | 0.4937 | 0.0000 | 1.0000 | 1.0000 |
| 11 | 11_65637273_A_G | 65637273 | 65869802 | rs501630 | G | A | 0.5556 | 0.0000 | 1.0000 | 1.0000 |
| 11 | 11_66337388_CA_ | 66337388 | 66569917 | 11:66337388_ | C | CA | 0.3372 | 0.0149 | 1.0150 | 1.0101 |
| | C | | | CA_C | | | | | | |
| 11 | 11_66694971_T_C | 66694971 | 66927500 | rs141065168 | C | T | 0.0867 | −0.0023 | 0.9977 | 0.9996 |
| 11 | 11_66751902_G_A | 66751902 | 66984431 | rs60197393 | A | G | 0.0543 | −0.0124 | 0.9877 | 0.9987 |
| 11 | 11_66825559_T_C | 66825559 | 67058088 | rs58876856 | C | T | 0.0853 | −0.0086 | 0.9914 | 0.9985 |
| 11 | 11_68959319_T_C | 68959319 | 69191851 | rs12800787 | C | T | 0.2972 | 0.0013 | 1.0013 | 1.0008 |
| 11 | 11_69289737_C_T | 69289737 | 69474969 | rs72927565 | T | C | 0.1282 | 0.0067 | 1.0067 | 1.0017 |
| 11 | 11_69322445_A_T | 69322445 | 69507677 | rs7952215 | T | A | 0.1035 | −0.0125 | 0.9876 | 0.9974 |
| 11 | 11_69330983_G_A | 69330983 | 69516215 | rs661204 | A | G | 0.1250 | 0.0760 | 1.0790 | 1.0198 |
| 11 | 11_69331418_C_T | 69331418 | 69516650 | rs78540526 | T | C | 0.0753 | 0.1358 | 1.1455 | 1.0220 |
| 11 | 11_69332712_C_T | 69332712 | 69517944 | rs657315 | T | C | 0.2157 | 0.0000 | 1.0000 | 1.0000 |
| 11 | 11_69334716_T_A | 69334716 | 69519948 | rs4608107 | A | T | 0.1627 | −0.0105 | 0.9896 | 0.9966 |
| 11 | 11_69343815_C_T | 69343815 | 69529047 | rs75296154 | T | C | 0.0423 | 0.0199 | 1.0201 | 1.0017 |
| 11 | 11_69352436_T_A | 69352436 | 69537668 | rs650524 | A | T | 0.8684 | 0.0052 | 1.0052 | 1.0091 |
| 11 | 11_69475386_C_T | 69475386 | 69660618 | rs147070975 | T | C | 0.0460 | 0.0057 | 1.0057 | 1.0005 |
| 11 | 11_69561529_A_G | 69561529 | 69746761 | rs9665984 | G | A | 0.6989 | 0.0070 | 1.0070 | 1.0098 |
| 11 | 11_69865341_GA_ | 69865341 | 70019235 | 11:69865341_ | G | GA | 0.7463 | 0.0024 | 1.0024 | 1.0036 |
| | G | | | GA_G | | | | | | |
| 11 | 11_72687549_G_T | 72687549 | 72976504 | rs573529 | T | G | 0.9898 | 0.0305 | 1.0310 | 1.0622 |
| 11 | 11_73101250_T_G | 73101250 | 73390205 | rs56411180 | G | T | 0.2363 | 0.0024 | 1.0024 | 1.0011 |
| 11 | 11_73186685_T_TA | 73186685 | 73475640 | rs141436441 | TA | T | 0.1038 | 0.0101 | 1.0102 | 1.0021 |
| 11 | 11_73348752_T_G | 73348752 | 73637707 | rs11235828 | G | T | 0.5592 | −0.0038 | 0.9962 | 0.9958 |
| 11 | 11_74042329_T_C | 74042329 | 74331284 | rs11236059 | C | T | 0.6350 | −0.0088 | 0.9912 | 0.9889 |
| 11 | 11_81598184_C_G | 81598184 | 81887142 | rs12418804 | G | C | 0.0690 | 0.0125 | 1.0126 | 1.0017 |
| 11 | 11_82445154_T_C | 82445154 | 82734112 | rs34030508 | C | T | 0.3603 | −0.0051 | 0.9949 | 0.9963 |
| 11 | 11_86277652_C_CC | 86277652 | 86566610 | rs10636674 | CCA | C | 0.2577 | 0.0096 | 1.0096 | 1.0050 |
| | A | | | | | | | | | |
| 11 | 11_87588165_C_A | 87588165 | 87877273 | rs117766181 | A | C | 0.0312 | −0.0066 | 0.9934 | 0.9996 |
| 11 | 11_91461188_A_G | 91461188 | 91728022 | rs794409 | G | A | 0.8227 | 0.0102 | 1.0103 | 1.0169 |
| 11 | 11_91711270_C_G | 91711270 | 91978104 | rs12295844 | G | C | 0.0702 | 0.0067 | 1.0067 | 1.0009 |
| 11 | 11_91717620_T_C | 91717620 | 91984454 | rs74735677 | C | T | 0.0254 | 0.0093 | 1.0093 | 1.0005 |
| 11 | 11_95526086_T_TA | 95526086 | 95792922 | rs35157937 | TA | T | 0.4061 | −0.0121 | 0.9880 | 0.9903 |
| 11 | 11_95884017_G_A | 95884017 | 96150853 | rs511141 | A | G | 0.3610 | −0.0129 | 0.9872 | 0.9908 |
| 11 | 11_97355695_T_G | 97355695 | 97484695 | rs10895838 | G | T | 0.6098 | 0.0015 | 1.0015 | 1.0018 |
| 11 | 11_97380744_A_A | 97380744 | 97509744 | rs139559507 | ATT | A | 0.1318 | −0.0142 | 0.9859 | 0.9963 |
| | TT | | | | | | | | | |
| 11 | 11_97403126_T_C | 97403126 | 97532126 | rs60829647 | C | T | 0.1818 | −0.0121 | 0.9880 | 0.9956 |
| 11 | 11_97414086_C_T | 97414086 | 97543086 | rs111239357 | T | C | 0.1173 | −0.0019 | 0.9981 | 0.9996 |
| 11 | 11_100885969_A_ | 100885969 | 101015238 | rs10895042 | G | A | 0.1584 | 0.0151 | 1.0152 | 1.0048 |
| | G | | | | | | | | | |
| 11 | 11_103614438_T_ | 103614438 | 103743710 | rs7125780 | G | T | 0.6572 | 0.0000 | 1.0000 | 1.0000 |
| | G | | | | | | | | | |
| 11 | 11_103647409_CT_ | 103647409 | 103776681 | 11:103647409 | C | CT | 0.5503 | 0.0088 | 1.0088 | 1.0098 |
| | C | | | CT_C | | | | | | |
| 11 | 11_103872193_T_C | 103872193 | 104001465 | rs61904889 | C | T | 0.0685 | −0.0040 | 0.9960 | 0.9995 |
| 11 | 11_106494949_C_T | 106494949 | 106624223 | rs72983856 | T | C | 0.1586 | −0.0070 | 0.9930 | 0.9978 |
| 11 | 11_107903818_A_ | 107903818 | 108033092 | rs36034326 | G | A | 0.0147 | 0.0000 | 1.0000 | 1.0000 |
| | G | | | | | | | | | |
| 11 | 11_107965390_A_ | 107965390 | 108094663 | rs71488291 | G | A | 0.0449 | 0.0000 | 1.0000 | 1.0000 |
| | G | | | | | | | | | |
| 11 | 11_108267402_C_C | 108267402 | 108396675 | rs199504893 | CA | C | 0.4173 | 0.0000 | 1.0000 | 1.0000 |
| | A | | | | | | | | | |
| 11 | 11_108267561_A_ | 108267561 | 108396834 | rs553909636 | AAAAT | A | 0.5482 | 0.0000 | 1.0000 | 1.0000 |
| | AAAAT | | | | | | | | | |
| 11 | 11_108406943_G_ | 108406943 | 108536216 | rs117706999 | A | G | 0.0243 | 0.0000 | 1.0000 | 1.0000 |
| | A | | | | | | | | | |
| 11 | 11_108431166_C_T | 108431166 | 108560439 | rs78142456 | T | C | 0.0130 | 0.0000 | 1.0000 | 1.0000 |
| 11 | 11_111117137_A_T | 111117137 | 111246412 | rs12803934 | T | A | 0.2323 | −0.0112 | 0.9889 | 0.9948 |
| 11 | 11_111120698_C_T | 111120698 | 111249973 | rs10891239 | T | C | 0.2794 | 0.0044 | 1.0044 | 1.0025 |
| 11 | 11_111170540_A_ | 111170540 | 111299815 | rs10891246 | G | A | 0.7194 | 0.0000 | 1.0000 | 1.0000 |
| | G | | | | | | | | | |
| 11 | 11_111188216_A_T | 111188216 | 111317491 | rs138219610 | T | A | 0.0213 | −0.0052 | 0.9948 | 0.9998 |
| 11 | 11_111195925_G_ | 111195925 | 111325200 | rs78790112 | A | G | 0.0621 | −0.0113 | 0.9888 | 0.9986 |
| | A | | | | | | | | | |
| 11 | 11_111220796_A_T | 111220796 | 111350071 | rs78904682 | T | A | 0.0219 | −0.0042 | 0.9958 | 0.9998 |
| 11 | 11_111415800_T_T | 111415800 | 111545075 | rs34170562 | TA | T | 0.3780 | −0.0088 | 0.9912 | 0.9934 |
| | A | | | | | | | | | |
| 11 | 11_111468326_C_T | 111468326 | 111597602 | rs145056657 | T | C | 0.0110 | −0.0218 | 0.9784 | 0.9995 |
| 11 | 11_111696440_T_C | 111696440 | 111825716 | rs610437 | C | T | 0.6221 | 0.0000 | 1.0000 | 1.0000 |
| 11 | 11_111742493_CA | 111742493 | 111871769 | | CAAAAA | CAAA | 0.3621 | 0.0113 | 1.0114 | 1.0082 |
| | AAA_CAAAAA | | | | | A | | | | |
| 11 | 11_112392948_C_T | 112392948 | 112522225 | rs11214235 | T | C | 0.0467 | −0.0034 | 0.9966 | 0.9997 |
| 11 | 11_113506860_C_T | 113506860 | 113636138 | rs10891576 | T | C | 0.8149 | 0.0026 | 1.0026 | 1.0042 |
| 11 | 11_113536710_G_ | 113536710 | 113665988 | rs10789956 | A | G | 0.7967 | 0.0024 | 1.0024 | 1.0038 |
| | A | | | | | | | | | |
| 11 | 11_113679119_C_T | 113679119 | 113808397 | rs2465647 | T | C | 0.8197 | 0.0041 | 1.0041 | 1.0067 |
| 11 | 11_113700546_CA_ | 113700546 | 113829824 | rs780916647 | C | CA | 0.6977 | 0.0008 | 1.0008 | 1.0011 |
| | C | | | | | | | | | |
| 11 | 11_113807181_G_ | 113807181 | 113936459 | rs11214775 | A | G | 0.2937 | −0.0044 | 0.9956 | 0.9974 |
| | A | | | | | | | | | |
| 11 | 11_113814744_CT_ | 113814744 | 113944022 | 11:113814744 | C | CT | 0.3475 | −0.0077 | 0.9923 | 0.9947 |
| | C | | | _CT_C | | | | | | |
| 11 | 11_114130424_AA | 114130424 | 114259702 | rs773108752 | A | AACT | 0.2377 | 0.0033 | 1.0033 | 1.0016 |
| | CT_A | | | | | | | | | |
| 11 | 11_114162880_T_C | 114162880 | 114292158 | rs73566515 | C | T | 0.0110 | −0.0160 | 0.9841 | 0.9997 |
| 11 | 11_115044211_C_T | 115044211 | 115173491 | rs4450197 | T | C | 0.9716 | 0.0026 | 1.0026 | 1.0051 |
| 11 | 11_115434178_T_T | 115434178 | 115563460 | rs35864364 | TTTTG | T | 0.6001 | 0.0108 | 1.0109 | 1.0131 |
| | TTTG | | | | | | | | | |
| 11 | 11_115447170_A_ | 115447170 | 115576452 | rs2446889 | G | A | 0.8291 | 0.0019 | 1.0019 | 1.0032 |
| | G | | | | | | | | | |
| 11 | 11_116684562_C_ | 116684562 | 116813846 | rs2727791 | G | C | 0.5259 | −0.0005 | 0.9995 | 0.9995 |
| | G | | | | | | | | | |
| 11 | 11_116724232_C_T | 116724232 | 116853516 | rs888246 | T | C | 0.1131 | −0.0084 | 0.9916 | 0.9981 |
| 11 | 11_116727936_A_T | 116727936 | 116857220 | rs625145 | T | A | 0.2046 | −0.0076 | 0.9924 | 0.9969 |
| 11 | 11_116883543_G_ | 116883543 | 117012827 | rs529358042 | GTT | G | 0.0475 | 0.0166 | 1.0167 | 1.0016 |
| | GTT | | | | | | | | | |
| 11 | 11_117074299_T_C | 117074299 | 117203583 | rs2269397 | C | T | 0.0935 | 0.0071 | 1.0071 | 1.0013 |
| 11 | 11_118568715_C_C | 118568715 | 118698006 | rs558343369 | CA | C | 0.2214 | −0.0119 | 0.9882 | 0.9948 |
| | A | | | | | | | | | |
| 11 | 11_120205001_G_ | 120205001 | 120334292 | rs34108129 | GT | G | 0.3948 | 0.0068 | 1.0068 | 1.0054 |
| | GT | | | | | | | | | |
| 11 | 11_122966626_A_ | 122966626 | 123095918 | rs7121616 | G | A | 0.2922 | −0.0150 | 0.9851 | 0.9913 |
| | G | | | | | | | | | |
| 11 | 11_123014229_C_C | 123014229 | 123143521 | rs571232271 | CG | C | 0.1556 | −0.0047 | 0.9953 | 0.9985 |
| | G | | | | | | | | | |
| 11 | 11_123053078_T_C | 123053078 | 123182370 | rs7113140 | C | T | 0.5648 | −0.0019 | 0.9981 | 0.9979 |
| 11 | 11_123136459_G_ | 123136459 | 123265751 | rs145464316 | A | G | 0.0176 | −0.0159 | 0.9842 | 0.9994 |
| | A | | | | | | | | | |
| 11 | 11_123910023_A_ | 123910023 | 124039316 | rs17128251 | G | A | 0.7284 | −0.0037 | 0.9963 | 0.9946 |
| | G | | | | | | | | | |
| 11 | 11_125291621_A_ | 125291621 | 125421725 | rs608529 | G | A | 0.1470 | 0.0008 | 1.0008 | 1.0002 |
| | G | | | | | | | | | |
| 11 | 11_125573435_CTT | 125573435 | 125703540 | rs113811036 | C | CTT | 0.1911 | −0.0057 | 0.9943 | 0.9978 |
| | C | | | | | | | | | |
| 11 | 11_125880063_C_T | 125880063 | 126010168 | rs55905554 | T | C | 0.1789 | 0.0114 | 1.0115 | 1.0041 |
| 11 | 11_129216251_A_ | 129216251 | 129346356 | rs111738532 | G | A | 0.0174 | 0.0058 | 1.0058 | 1.0002 |
| | G | | | | | | | | | |
| 11 | 11_129243417_T_ | 129243417 | 129373522 | rs7939702 | G | T | 0.8620 | −0.0105 | 0.9896 | 0.9821 |
| | G | | | | | | | | | |
| 11 | 11_129248862_G_ | 129248862 | 129378967 | rs6590383 | A | G | 0.8258 | −0.0087 | 0.9913 | 0.9857 |
| | A | | | | | | | | | |
| 11 | 11_129360748_AT_ | 129360748 | 129490853 | rs774162920 | A | AT | 0.1562 | 0.0118 | 1.0119 | 1.0037 |
| | A | | | | | | | | | |
| 11 | 11_129452507_C_ | 129452507 | 129582612 | | G | C | 0.6171 | 0.0145 | 1.0146 | 1.0181 |
| | G | | | | | | | | | |
| 11 | 11_129457822_A_C | 129457822 | 129587927 | rs11221768 | C | A | 0.5541 | 0.0003 | 1.0003 | 1.0003 |
| 11 | 11_129461016_A_ | 129461016 | 129591121 | rs11822830 | G | A | 0.6016 | 0.0115 | 1.0116 | 1.0140 |
| | G | | | | | | | | | |
| 11 | 11_129490917_G_ | 129490917 | 129621022 | rs7930630 | A | G | 0.1906 | 0.0052 | 1.0052 | 1.0020 |
| | A | | | | | | | | | |
| 11 | 11_129537177_C_T | 129537177 | 129667282 | rs11221788 | T | C | 0.1872 | 0.0103 | 1.0104 | 1.0039 |
| 11 | 11_129650014_T_A | 129650014 | 129780119 | rs1940109 | A | T | 0.7790 | −0.0030 | 0.9970 | 0.9953 |
| 11 | 11_129723021_T_C | 129723021 | 129853126 | rs692311 | C | T | 0.1762 | 0.0011 | 1.0011 | 1.0004 |
| 11 | 11_129723158_G_ | 129723158 | 129853263 | rs692327 | A | G | 0.5180 | −0.0038 | 0.9962 | 0.9961 |
| | A | | | | | | | | | |
| 11 | 11_130308727_C_T | 130308727 | 130438832 | rs61913896 | T | C | 0.0390 | −0.0179 | 0.9823 | 0.9986 |
| 11 | 11_130347681_A_ | 130347681 | 130477786 | rs34418043 | G | A | 0.3365 | −0.0067 | 0.9933 | 0.9955 |
| | G | | | | | | | | | |
| 11 | 11_130687075_C_T | 130687075 | 130817180 | rs11222330 | T | C | 0.4605 | 0.0057 | 1.0057 | 1.0053 |
| 11 | 11_131352969_A_ | 131352969 | 131483075 | rs10791148 | G | A | 0.3949 | 0.0115 | 1.0116 | 1.0092 |
| | G | | | | | | | | | |
| 11 | 11_131961491_C_T | 131961491 | 132091597 | rs116944485 | T | C | 0.0177 | −0.0155 | 0.9846 | 0.9995 |
| 11 | 11_132372467_T_C | 132372467 | 132502573 | rs11223094 | C | T | 0.0325 | −0.0342 | 0.9664 | 0.9978 |
| 11 | 11_132732213_T_C | 132732213 | 132862318 | rs6590660 | C | T | 0.3128 | 0.0000 | 1.0000 | 1.0000 |
| 11 | 11_132744115_T_A | 132744115 | 132874220 | rs2725423 | A | T | 0.3002 | 0.0000 | 1.0000 | 1.0000 |
| 11 | 11_134220859_T_A | 134220859 | 134350965 | rs61908675 | A | T | 0.1870 | 0.0105 | 1.0106 | 1.0040 |
| 12 | 12_191450_T_C | 191450 | 82284 | rs7313694 | C | T | 0.5523 | 0.0035 | 1.0035 | 1.0039 |
| 12 | 12_293626_A_G | 293626 | 184460 | rs797736 | G | A | 0.3711 | 0.0125 | 1.0126 | 1.0094 |
| 12 | 12_348419_T_C | 348419 | 239253 | rs10774019 | C | T | 0.8959 | 0.0106 | 1.0107 | 1.0192 |
| 12 | 12_1045023_AAAA | 1045023 | 935857 | 12:1045023_A | A | AAAA | 0.7525 | 0.0097 | 1.0097 | 1.0147 |
| | T_A | | | AAAT_A | | T | | | | |
| 12 | 12_1593428_A_G | 1593428 | 1484262 | rs61912164 | G | A | 0.0440 | −0.0098 | 0.9902 | 0.9991 |
| 12 | 12_2294263_A_G | 2294263 | 2185097 | rs758171 | G | A | 0.3750 | 0.0005 | 1.0005 | 1.0004 |
| 12 | 12_2340798_A_C | 2340798 | 2231632 | rs34382810 | C | A | 0.4235 | 0.0085 | 1.0085 | 1.0072 |
| 12 | 12_5638925_G_GG | 5638925 | 5529759 | rs149293159 | GGACCTC | G | 0.2378 | 0.0101 | 1.0102 | 1.0048 |
| | ACCTCCCACCAGA | | | | CCACCAG | | | | | |
| | | | | | A | | | | | |
| 12 | 12_5665330_A_T | 5665330 | 5556164 | rs11063753 | T | A | 0.2427 | 0.0012 | 1.0012 | 1.0006 |
| 12 | 12_6294636_A_G | 6294636 | 6185470 | rs80286123 | G | A | 0.5265 | 0.0020 | 1.0020 | 1.0021 |
| 12 | 12_12008900_G_T | 12008900 | 11855966 | rs80046954 | T | G | 0.0524 | −0.0045 | 0.9955 | 0.9995 |
| 12 | 12_12044393_T_TT | 12044393 | 11891459 | rs71435871 | TTA | T | 0.3140 | −0.0023 | 0.9977 | 0.9986 |
| | A | | | | | | | | | |
| 12 | 12_12575921_T_TA | 12575921 | 12422987 | rs113936393 | TA | T | 0.2064 | 0.0005 | 1.0005 | 1.0002 |
| 12 | 12_12710877_G_T | 12710877 | 12557943 | rs74945783 | T | G | 0.0807 | 0.0259 | 1.0262 | 1.0042 |
| 12 | 12_12871099_T_G | 12871099 | 12718165 | rs2066827 | G | T | 0.2387 | −0.0015 | 0.9985 | 0.9993 |
| 12 | 12_13144492_C_T | 13144492 | 12991558 | rs11834741 | T | C | 0.0615 | −0.0031 | 0.9969 | 0.9996 |
| 12 | 12_13191493_C_C | 13191493 | 13038559 | rs3080953 | CAA | C | 0.7517 | 0.0002 | 1.0002 | 1.0003 |
| | AA | | | | | | | | | |
| 12 | 12_13270924_T_C | 13270924 | 13117990 | rs10845705 | C | T | 0.2541 | −0.0062 | 0.9938 | 0.9969 |
| 12 | 12_13337385_G_A | 13337385 | 13184451 | rs6488595 | A | G | 0.8931 | −0.0139 | 0.9862 | 0.9755 |
| 12 | 12_13360231_G_A | 13360231 | 13207297 | rs74063269 | A | G | 0.1112 | 0.0003 | 1.0003 | 1.0001 |
| 12 | 12_14408554_G_C | 14408554 | 14255620 | rs75905550 | C | G | 0.1956 | 0.0159 | 1.0160 | 1.0063 |
| 12 | 12_14413931_G_C | 14413931 | 14260997 | rs12422552 | C | G | 0.2619 | 0.0093 | 1.0093 | 1.0049 |
| 12 | 12_14730966_T_A | 14730966 | 14578032 | rs78973331 | A | T | 0.1545 | 0.0034 | 1.0034 | 1.0011 |
| 12 | 12_15308279_G_A | 15308279 | 15155345 | rs67581511 | A | G | 0.2423 | 0.0074 | 1.0074 | 1.0036 |
| 12 | 12_17591303_A_G | 17591303 | 17438369 | rs1163944 | G | A | 0.7418 | 0.0014 | 1.0014 | 1.0021 |
| 12 | 12_18250884_G_T | 18250884 | 18097950 | rs111219473 | T | G | 0.1234 | 0.0159 | 1.0160 | 1.0040 |
| 12 | 12_18398806_T_C | 18398806 | 18245872 | rs10505814 | C | T | 0.7008 | 0.0057 | 1.0057 | 1.0080 |
| 12 | 12_18459387_C_T | 18459387 | 18306453 | rs12582971 | T | C | 0.1331 | −0.0037 | 0.9963 | 0.9990 |
| 12 | 12_20645903_G_T | 20645903 | 20492969 | rs7295612 | T | G | 0.6845 | 0.0011 | 1.0011 | 1.0015 |
| 12 | 12_20650951_C_T | 20650951 | 20498017 | rs7484304 | T | C | 0.2491 | −0.0105 | 0.9896 | 0.9948 |
| 12 | 12_20650985_C_T | 20650985 | 20498051 | rs7131827 | T | C | 0.3039 | 0.0050 | 1.0050 | 1.0030 |
| 12 | 12_25189664_TTTG | 25189664 | 25036730 | rs757757128 | T | TTTG | 0.0313 | −0.0249 | 0.9754 | 0.9985 |
| | _T | | | | | | | | | |
| 12 | 12_28045047_A_G | 28045047 | 27892114 | rs111798596 | G | A | 0.0233 | −0.0140 | 0.9861 | 0.9994 |
| 12 | 12_28139846_T_C | 28139846 | 27986913 | rs805510 | C | T | 0.8782 | 0.0302 | 1.0307 | 1.0546 |
| 12 | 12_28149568_C_T | 28149568 | 27996635 | rs788458 | T | C | 0.1170 | −0.0094 | 0.9906 | 0.9978 |
| 12 | 12_28151609_A_C | 28151609 | 27998676 | rs812020 | C | A | 0.2759 | −0.0167 | 0.9834 | 0.9909 |
| 12 | 12_28152993_G_A | 28152993 | 28000060 | rs805583 | A | G | 0.3033 | −0.0027 | 0.9973 | 0.9984 |
| 12 | 12_28157711_G_A | 28157711 | 28004778 | rs11049266 | A | G | 0.0151 | 0.0000 | 1.0000 | 1.0000 |
| 12 | 12_28174817_C_T | 28174817 | 28021884 | rs7297051 | T | C | 0.2421 | −0.0506 | 0.9507 | 0.9762 |
| 12 | 12_28183286_A_G | 28183286 | 28030353 | rs7976725 | G | A | 0.2321 | −0.0029 | 0.9971 | 0.9987 |
| 12 | 12_28221970_T_A | 28221970 | 28069037 | rs6487658 | A | T | 0.4614 | −0.0044 | 0.9956 | 0.9960 |
| 12 | 12_28258195_G_T | 28258195 | 28105262 | rs185286466 | T | G | 0.0189 | 0.0077 | 1.0077 | 1.0003 |
| 12 | 12_28304439_T_C | 28304439 | 28151506 | rs1600346 | C | T | 0.3603 | −0.0227 | 0.9776 | 0.9839 |
| 12 | 12_28347382_C_T | 28347382 | 28194449 | rs11049431 | T | C | 0.2153 | −0.0021 | 0.9979 | 0.9991 |
| 12 | 12_29000608_C_T | 29000608 | 28847675 | rs5018009 | T | C | 0.8697 | 0.0031 | 1.0031 | 1.0054 |
| 12 | 12_29140260_G_A | 29140260 | 28987327 | rs1027113 | A | G | 0.9126 | 0.0357 | 1.0363 | 1.0674 |
| 12 | 12_29229856_G_A | 29229856 | 29076923 | rs10771479 | A | G | 0.8569 | −0.0028 | 0.9972 | 0.9952 |
| 12 | 12_29352686_G_A | 29352686 | 29199753 | rs10743648 | A | G | 0.8415 | −0.0034 | 0.9966 | 0.9943 |
| 12 | 12_29489004_A_G | 29489004 | 29336071 | rs77301656 | G | A | 0.0483 | −0.0228 | 0.9775 | 0.9978 |
| 12 | 12_29634797_T_A | 29634797 | 29481864 | rs35049626 | A | T | 0.0319 | −0.0080 | 0.9920 | 0.9995 |
| 12 | 12_29964996_T_C | 29964996 | 29812063 | rs566253232 | C | T | 0.0233 | −0.0059 | 0.9941 | 0.9997 |
| 12 | 12_31276226_G_A | 31276226 | 31123292 | rs7133957 | A | G | 0.1029 | −0.0121 | 0.9880 | 0.9975 |
| 12 | 12_31522408_A_C | 31522408 | 31369474 | rs708205 | C | A | 0.3605 | 0.0049 | 1.0049 | 1.0035 |
| 12 | 12_31763639_G_A | 31763639 | 31610705 | rs7310240 | A | G | 0.5441 | −0.0057 | 0.9943 | 0.9938 |
| 12 | 12_32362886_G_A | 32362886 | 32209952 | rs3850981 | A | G | 0.0799 | −0.0130 | 0.9871 | 0.9979 |
| 12 | 12_32843389_G_C | 32843389 | 32690455 | rs73082818 | C | G | 0.0215 | −0.0195 | 0.9807 | 0.9992 |
| 12 | 12_42498057_A_T | 42498057 | 42104255 | rs34619182 | T | A | 0.1824 | 0.0041 | 1.0041 | 1.0015 |
| 12 | 12_42528698_T_G | 42528698 | 42134896 | rs3852593 | G | T | 0.3560 | 0.0059 | 1.0059 | 1.0042 |
| 12 | 12_46522194_A_A | 46522194 | 46128411 | | AGTATTC | A | 0.2437 | −0.0016 | 0.9984 | 0.9992 |
| | GTATTCTATG | | | | TATG | | | | | |
| 12 | 12_48251076_C_T | 48251076 | 47857293 | rs12721367 | T | C | 0.0227 | 0.0139 | 1.0140 | 1.0006 |
| 12 | 12_49123316_C_C | 49123316 | 48729533 | rs149424542 | CA | C | 0.2958 | 0.0079 | 1.0079 | 1.0047 |
| | A | | | | | | | | | |
| 12 | 12_50006464_A_C | 50006464 | 49612681 | rs117792145 | C | A | 0.0108 | 0.0174 | 1.0176 | 1.0004 |
| 12 | 12_53931047_TTCT | 53931047 | 53537263 | 12:53931047_ | T | TTCT | 0.0240 | 0.0132 | 1.0133 | 1.0006 |
| | CCATG_T | | | TTCTCCATG_T | | CCAT | | | | |
| | | | | | | G | | | | |
| 12 | 12_54958164_C_G | 54958164 | 54564380 | rs7953703 | G | C | 0.2905 | 0.0022 | 1.0022 | 1.0013 |
| 12 | 12_54967901_C_T | 54967901 | 54574117 | rs3782411 | T | C | 0.3142 | 0.0036 | 1.0036 | 1.0023 |
| 12 | 12_55047126_G_A | 55047126 | 54653342 | rs11171005 | A | G | 0.2241 | −0.0016 | 0.9984 | 0.9993 |
| 12 | 12_56438149_T_TG | 56438149 | 56044365 | rs59385427 | TG | T | 0.9371 | −0.0066 | 0.9934 | 0.9877 |
| 12 | 12_56493822_A_C | 56493822 | 56100038 | rs2292238 | C | A | 0.4123 | −0.0076 | 0.9924 | 0.9938 |
| 12 | 12_57146069_T_G | 57146069 | 56752285 | rs2277339 | G | T | 0.1037 | −0.0212 | 0.9790 | 0.9957 |
| 12 | 12_57836098_A_G | 57836098 | 57442315 | rs2943692 | G | A | 0.2404 | −0.0086 | 0.9914 | 0.9959 |
| 12 | 12_57847938_G_A | 57847938 | 57454155 | rs703845 | A | G | 0.2773 | −0.0052 | 0.9948 | 0.9971 |
| 12 | 12_57887130_ATA | 57887130 | 57493347 | rs756746321 | A | ATAT | 0.1301 | −0.0047 | 0.9953 | 0.9988 |
| | TATAT_A | | | | | ATAT | | | | |
| 12 | 12_58091039_T_G | 58091039 | 57697256 | rs17455486 | G | T | 0.0291 | −0.0106 | 0.9895 | 0.9994 |
| 12 | 12_58274737_G_T | 58274737 | 57880954 | rs147072673 | T | G | 0.0132 | 0.0240 | 1.0243 | 1.0006 |
| 12 | 12_58912426_C_T | 58912426 | 58518643 | rs79808633 | T | C | 0.0251 | −0.0097 | 0.9903 | 0.9995 |
| 12 | 12_64105782_TA_T | 64105782 | 63712002 | | TAA | TA | 0.1771 | 0.0031 | 1.0031 | 1.0011 |
| | AA | | | | | | | | | |
| 12 | 12_64318956_C_A | 64318956 | 63925176 | rs61933130 | A | C | 0.0358 | −0.0280 | 0.9724 | 0.9980 |
| 12 | 12_64428763_T_G | 64428763 | 64034983 | rs73130130 | G | T | 0.2550 | 0.0085 | 1.0085 | 1.0044 |
| 12 | 12_64531763_A_T | 64531763 | 64137983 | rs12831194 | T | A | 0.5177 | −0.0020 | 0.9980 | 0.9979 |
| 12 | 12_68002302_G_C | 68002302 | 67608522 | rs11176802 | C | G | 0.1429 | 0.0062 | 1.0062 | 1.0018 |
| 12 | 12_68112905_A_G | 68112905 | 67719125 | rs9652024 | G | A | 0.6277 | −0.0079 | 0.9921 | 0.9901 |
| 12 | 12_68199301_A_G | 68199301 | 67805521 | rs116654690 | G | A | 0.0224 | −0.0442 | 0.9568 | 0.9981 |
| 12 | 12_68554461_A_G | 68554461 | 68160681 | rs2069732 | G | A | 0.0367 | −0.0098 | 0.9902 | 0.9993 |
| 12 | 12_69100408_C_T | 69100408 | 68706628 | rs2546522 | T | C | 0.2749 | −0.0081 | 0.9919 | 0.9956 |
| 12 | 12_69363979_C_T | 69363979 | 68970199 | rs117531987 | T | C | 0.0290 | 0.0048 | 1.0048 | 1.0003 |
| 12 | 12_69418116_A_G | 69418116 | 69024336 | rs55836995 | G | A | 0.0909 | −0.0082 | 0.9918 | 0.9985 |
| 12 | 12_69551106_T_C | 69551106 | 69157326 | rs78574703 | C | T | 0.0575 | 0.0120 | 1.0121 | 1.0014 |
| 12 | 12_69553408_A_C | 69553408 | 69159628 | rs61926895 | C | A | 0.0988 | 0.0030 | 1.0030 | 1.0006 |
| 12 | 12_69676379_TTA_ | 69676379 | 69282599 | 12:69676379_ | T | TTA | 0.6234 | 0.0074 | 1.0074 | 1.0093 |
| | T | | | TTA_T | | | | | | |
| 12 | 12_69823491_T_G | 69823491 | 69429711 | rs17106576 | G | T | 0.2477 | 0.0018 | 1.0018 | 1.0009 |
| 12 | 12_70088344_T_C | 70088344 | 69694564 | rs75229567 | C | T | 0.0530 | −0.0050 | 0.9950 | 0.9995 |
| 12 | 12_70798355_A_T | 70798355 | 70404575 | rs2870876 | T | A | 0.1810 | 0.0145 | 1.0146 | 1.0053 |
| 12 | 12_70809003_C_G | 70809003 | 70415223 | rs28672988 | G | C | 0.2422 | 0.0012 | 1.0012 | 1.0006 |
| 12 | 12_72324371_G_C | 72324371 | 71930591 | rs4131347 | C | G | 0.2225 | −0.0073 | 0.9927 | 0.9968 |
| 12 | 12_72391545_GA_ | 72391545 | 71997765 | 12:72391545_ | G | GA | 0.2647 | 0.0000 | 1.0000 | 1.0000 |
| | G | | | GA G | | | | | | |
| 12 | 12_77429681_TA_T | 77429681 | 77035901 | 12:77429681_ | T | TA | 0.5275 | 0.0085 | 1.0085 | 1.0090 |
| | | | | TA_T | | | | | | |
| 12 | 12_77445728_T_A | 77445728 | 77051948 | rs74650509 | A | T | 0.1068 | 0.0020 | 1.0020 | 1.0004 |
| 12 | 12_80441323_A_G | 80441323 | 80047543 | rs59956390 | G | A | 0.0245 | −0.0125 | 0.9876 | 0.9994 |
| 12 | 12_82472014_T_A | 82472014 | 82078235 | rs7979964 | A | T | 0.5239 | 0.0013 | 1.0013 | 1.0014 |
| 12 | 12_83064195_G_G | 83064195 | 82670416 | rs111622698 | GA | G | 0.0992 | 0.0115 | 1.0116 | 1.0023 |
| | A | | | | | | | | | |
| 12 | 12_83085107_A_A | 83085107 | 82691328 | rs111797688 | AT | A | 0.1178 | 0.0050 | 1.0050 | 1.0012 |
| | T | | | | | | | | | |
| 12 | 12_83105773_T_C | 83105773 | 82711994 | rs7134766 | C | T | 0.8773 | −0.0106 | 0.9895 | 0.9816 |
| 12 | 12_84692840_G_G | 84692840 | 84299061 | rs10677679 | GTA | G | 0.2820 | 0.0028 | 1.0028 | 1.0016 |
| | TA | | | | | | | | | |
| 12 | 12_85004551_C_T | 85004551 | 84610772 | rs10862899 | T | C | 0.4955 | 0.0086 | 1.0086 | 1.0086 |
| 12 | 12_85009437_T_C | 85009437 | 84615658 | rs202049448 | C | T | 0.3360 | −0.0069 | 0.9931 | 0.9954 |
| 12 | 12_85019560_G_A | 85019560 | 84625781 | rs73365443 | A | G | 0.1936 | −0.0008 | 0.9992 | 0.9997 |
| 12 | 12_85024975_G_A | 85024975 | 84631196 | rs11116506 | A | G | 0.1721 | −0.0001 | 0.9999 | 1.0000 |
| 12 | 12_87480758_ATCT | 87480758 | 87086981 | 12:87480758 | A | ATCT | 0.1753 | −0.0069 | 0.9931 | 0.9976 |
| | _A | | | ATCT_A | | | | | | |
| 12 | 12_88165225_T_C | 88165225 | 87771448 | rs77205452 | C | T | 0.0225 | −0.0076 | 0.9924 | 0.9997 |
| 12 | 12_88253612_AAA | 88253612 | 87859835 | rs774846867 | A | AAAA | 0.6417 | 0.0000 | 1.0000 | 1.0000 |
| | AATAGACCTTCCAA | | | | | ATAG | | | | |
| | AT_A | | | | | ACCT | | | | |
| | | | | | | TCCA | | | | |
| | | | | | | AAT | | | | |
| 12 | 12_92251023_A_G | 92251023 | 91857247 | rs67075180 | G | A | 0.0302 | 0.0188 | 1.0190 | 1.0011 |
| 12 | 12_92948635_T_C | 92948635 | 92554859 | rs116937902 | C | T | 0.0145 | −0.0067 | 0.9933 | 0.9998 |
| 12 | 12_93190239_T_TT | 93190239 | 92796463 | rs144029000 | TTA | T | 0.4649 | −0.0016 | 0.9984 | 0.9985 |
| | A | | | | | | | | | |
| 12 | 12_93478349_A_C | 93478349 | 93084573 | rs142852759 | C | A | 0.0181 | −0.0088 | 0.9912 | 0.9997 |
| 12 | 12_93958657_A_G | 93958657 | 93564881 | rs10859531 | G | A | 0.1047 | 0.0044 | 1.0044 | 1.0009 |
| 12 | 12_94661453_A_G | 94661453 | 94267677 | rs61708525 | G | A | 0.2937 | 0.0047 | 1.0047 | 1.0028 |
| 12 | 12_94661511_T_G | 94661511 | 94267735 | rs56725874 | G | T | 0.1144 | 0.0006 | 1.0006 | 1.0001 |
| 12 | 12_94672888_G_A | 94672888 | 94279112 | rs832504 | A | G | 0.8467 | −0.0045 | 0.9955 | 0.9924 |
| 12 | 12_95407517_C_T | 95407517 | 95013741 | rs138868138 | T | C | 0.0205 | −0.0208 | 0.9794 | 0.9992 |
| 12 | 12_95906139_C_T | 95906139 | 95512363 | rs71458596 | T | C | 0.0267 | −0.0102 | 0.9899 | 0.9995 |
| 12 | 12_96021033_T_C | 96021033 | 95627257 | rs2367556 | C | T | 0.5086 | −0.0010 | 0.9990 | 0.9990 |
| 12 | 12_96022099_G_C | 96022099 | 95628323 | rs12307566 | C | G | 0.5710 | −0.0069 | 0.9931 | 0.9922 |
| 12 | 12_96027467_C_T | 96027467 | 95633691 | rs11836367 | T | C | 0.3436 | −0.0329 | 0.9676 | 0.9779 |
| 12 | 12_96027759_A_G | 96027759 | 95633983 | rs17356907 | G | A | 0.2963 | −0.0267 | 0.9737 | 0.9844 |
| 12 | 12_96028525_A_G | 96028525 | 95634749 | rs1833472 | G | A | 0.9296 | −0.0056 | 0.9944 | 0.9896 |
| 12 | 12_96141866_A_G | 96141866 | 95748088 | rs117374882 | G | A | 0.1717 | −0.0012 | 0.9988 | 0.9996 |
| 12 | 12_96243051_G_A | 96243051 | 95849273 | rs76415780 | A | G | 0.0498 | 0.0112 | 1.0113 | 1.0011 |
| 12 | 12_97221040_G_A | 97221040 | 96827262 | rs62644119 | A | G | 0.7167 | 0.0043 | 1.0043 | 1.0062 |
| 12 | 12_97283972_T_C | 97283972 | 96890194 | rs918105 | C | T | 0.4619 | 0.0053 | 1.0053 | 1.0049 |
| 12 | 12_97606385_C_T | 97606385 | 97212607 | rs6538789 | T | C | 0.0297 | −0.0147 | 0.9854 | 0.9991 |
| 12 | 12_103097070_A_ | 103097070 | 102703292 | rs7306173 | G | A | 0.1148 | 0.0140 | 1.0141 | 1.0032 |
| | G | | | | | | | | | |
| 12 | 12_103101412_T_C | 103101412 | 102707634 | rs1463770 | C | T | 0.7164 | −0.0057 | 0.9943 | 0.9919 |
| 12 | 12_105978901_C_T | 105978901 | 105585123 | rs11112601 | T | C | 0.0303 | 0.0131 | 1.0132 | 1.0008 |
| 12 | 12_106022283_A_ | 106022283 | 105628505 | rs200487971 | AGGGGT | A | 0.5686 | 0.0030 | 1.0030 | 1.0034 |
| | AGGGGT | | | | | | | | | |
| 12 | 12_106421904_C_T | 106421904 | 106028126 | rs76487198 | T | C | 0.1013 | −0.0014 | 0.9986 | 0.9997 |
| 12 | 12_108098065_G_ | 108098065 | 107704288 | rs67446565 | C | G | 0.1602 | −0.0056 | 0.9944 | 0.9982 |
| | C | | | | | | | | | |
| 12 | 12_109905720_G_ | 109905720 | 109467915 | rs9943764 | A | G | 0.1806 | −0.0073 | 0.9927 | 0.9974 |
| | A | | | | | | | | | |
| 12 | 12_111189543_T_ | 111189543 | 110751738 | rs10161511 | G | T | 0.7724 | −0.0017 | 0.9983 | 0.9974 |
| | G | | | | | | | | | |
| 12 | 12_111288843_T_C | 111288843 | 110851039 | rs10219717 | C | T | 0.6135 | 0.0013 | 1.0013 | 1.0016 |
| 12 | 12_111349849_A_ | 111349849 | 110912045 | rs11065766 | G | A | 0.3151 | 0.0027 | 1.0027 | 1.0017 |
| | G | | | | | | | | | |
| 12 | 12_111427783_GT | 111427783 | 110989979 | 12:111427783 | G | GT | 0.6130 | 0.0034 | 1.0034 | 1.0042 |
| | _G | | | _GT_G | | | | | | |
| 12 | 12_111600134_G_ | 111600134 | 111162330 | rs11065822 | T | G | 0.3715 | −0.0188 | 0.9814 | 0.9862 |
| | T | | | | | | | | | |
| 12 | 12_111712755_CA_ | 111712755 | 111274951 | rs771598754 | C | CA | 0.2212 | 0.0003 | 1.0003 | 1.0001 |
| | C | | | | | | | | | |
| 12 | 12_111826477_A_ | 111826477 | 111388673 | rs111338191 | AAATT | A | 0.5074 | 0.0053 | 1.0053 | 1.0054 |
| | AAATT | | | | | | | | | |
| 12 | 12_113028429_G_ | 113028429 | 112590625 | rs67436553 | A | G | 0.0239 | 0.0018 | 1.0018 | 1.0001 |
| | A | | | | | | | | | |
| 12 | 12_113032969_TA | 113032969 | 112595165 | 12:113032969 | T | TA | 0.5827 | 0.0016 | 1.0016 | 1.0019 |
| | T | | | _TA_T | | | | | | |
| 12 | 12_114108404_T_C | 114108404 | 113670599 | rs535239 | C | T | 0.0184 | 0.0019 | 1.0019 | 1.0001 |
| 12 | 12_115092018_A_T | 115092018 | 114654213 | rs35383422 | T | A | 0.2493 | 0.0000 | 1.0000 | 1.0000 |
| 12 | 12_115108136_T_C | 115108136 | 114670331 | rs1061657 | C | T | 0.2615 | 0.0026 | 1.0026 | 1.0014 |
| 12 | 12_115109223_C_ | 115109223 | 114671418 | rs3741698 | G | C | 0.2829 | 0.0219 | 1.0221 | 1.0126 |
| | G | | | | | | | | | |
| 12 | 12_115364215_A_ | 115364215 | 114926410 | rs75843093 | G | A | 0.0372 | 0.0062 | 1.0062 | 1.0005 |
| | G | | | | | | | | | |
| 12 | 12_115370514_CA_ | 115370514 | 114932709 | 12:115370514 | C | CA | 0.7071 | −0.0013 | 0.9987 | 0.9982 |
| | C | | | _CA_C | | | | | | |
| 12 | 12_115374162_G_ | 115374162 | 114936357 | rs2891538 | A | G | 0.4143 | −0.0043 | 0.9957 | 0.9964 |
| | A | | | | | | | | | |
| 12 | 12_115478150_G_ | 115478150 | 115040345 | rs35515 | A | G | 0.0559 | −0.0252 | 0.9751 | 0.9972 |
| | A | | | | | | | | | |
| 12 | 12_115558444_G_ | 115558444 | 115120639 | rs7970854 | C | G | 0.3370 | 0.0000 | 1.0000 | 1.0000 |
| | C | | | | | | | | | |
| 12 | 12_115570046_G_ | 115570046 | 115132241 | rs35426 | A | G | 0.5793 | 0.0045 | 1.0045 | 1.0052 |
| | A | | | | | | | | | |
| 12 | 12_115583800_G_ | 115583800 | 115145995 | rs35423 | A | G | 0.5037 | 0.0000 | 1.0000 | 1.0000 |
| | A | | | | | | | | | |
| 12 | 12_115710648_G | 115710648 | 115272843 | rs1155698 | A | G | 0.6599 | 0.0000 | 1.0000 | 1.0000 |
| | A | | | | | | | | | |
| 12 | 12_115775595_C_T | 115775595 | 115337790 | rs74959053 | T | C | 0.1050 | 0.0135 | 1.0136 | 1.0029 |
| 12 | 12_115789327_T_C | 115789327 | 115351522 | rs4767312 | C | T | 0.1432 | 0.0000 | 1.0000 | 1.0000 |
| 12 | 12_115796577_A_ | 115796577 | 115358772 | rs11067551 | G | A | 0.1959 | −0.0235 | 0.9768 | 0.9909 |
| | G | | | | | | | | | |
| 12 | 12_115831911_GA | 115831911 | 115394106 | 12:115831911 | G | GA | 0.1628 | 0.0000 | 1.0000 | 1.0000 |
| | _G | | | _GA G | | | | | | |
| 12 | 12_115834946_C_ | 115834946 | 115397141 | rs2133317 | G | C | 0.3827 | −0.0046 | 0.9954 | 0.9965 |
| | G | | | | | | | | | |
| 12 | 12_115835836_T_C | 115835836 | 115398031 | rs2454399 | C | T | 0.4171 | −0.0458 | 0.9552 | 0.9630 |
| 12 | 12_115837032_T_T | 115837032 | 115399227 | | TA | T | 0.3914 | −0.0083 | 0.9917 | 0.9935 |
| | A | | | | | | | | | |
| 12 | 12_115838544_T_A | 115838544 | 115400739 | rs10466929 | A | T | 0.4886 | 0.0010 | 1.0010 | 1.0010 |
| 12 | 12_115852376_A_C | 115852376 | 115414571 | rs7979538 | C | A | 0.2902 | 0.0047 | 1.0047 | 1.0027 |
| 12 | 12_115916851_C_C | 115916851 | 115479046 | rs576675881 | CA | C | 0.0645 | −0.0074 | 0.9926 | 0.9990 |
| | A | | | | | | | | | |
| 12 | 12_115935967_C_T | 115935967 | 115498162 | rs57863152 | T | C | 0.0444 | 0.0000 | 1.0000 | 1.0000 |
| 12 | 12_115937885_T_C | 115937885 | 115500080 | rs66707136 | C | T | 0.0518 | −0.0213 | 0.9789 | 0.9978 |
| 12 | 12_116017035_A_ | 116017035 | 115579230 | rs7953921 | G | A | 0.5045 | 0.0008 | 1.0008 | 1.0008 |
| | G | | | | | | | | | |
| 12 | 12_116347863_A_ | 116347863 | 115910058 | rs12296461 | G | A | 0.4791 | −0.0048 | 0.9952 | 0.9954 |
| | G | | | | | | | | | |
| 12 | 12_116757287_A_T | 116757287 | 116319482 | rs4767396 | T | A | 0.3410 | −0.0133 | 0.9868 | 0.9910 |
| 12 | 12_117068276_C_ | 117068276 | 116630471 | rs12814231 | G | C | 0.3478 | 0.0035 | 1.0035 | 1.0024 |
| | G | | | | | | | | | |
| 12 | 12_117071777_C_T | 117071777 | 116633972 | rs12823501 | T | C | 0.4735 | 0.0011 | 1.0011 | 1.0010 |
| 12 | 12_120793690_G_ | 120793690 | 120355887 | rs56655594 | GGAA | G | 0.1619 | 0.0009 | 1.0009 | 1.0003 |
| | GGAA | | | | | | | | | |
| 12 | 12_120832146_C_T | 120832146 | 120394343 | rs206966 | T | C | 0.1593 | 0.0216 | 1.0218 | 1.0070 |
| 12 | 12_121416622_C_ | 121416622 | 120978819 | rs1169289 | G | C | 0.4556 | −0.0078 | 0.9922 | 0.9929 |
| | G | | | | | | | | | |
| 12 | 12_121450384_G_ | 121450384 | 121012581 | rs2254971 | C | G | 0.3843 | −0.0019 | 0.9981 | 0.9985 |
| | C | | | | | | | | | |
| 12 | 12_122717710_C_A | 122717710 | 122233163 | rs117523992 | A | C | 0.0253 | −0.0066 | 0.9934 | 0.9997 |
| 12 | 12_123430082_C_T | 123430082 | 122945535 | rs12815479 | T | C | 0.0840 | −0.0109 | 0.9892 | 0.9982 |
| 12 | 12_123896851_G_ | 123896851 | 123412304 | rs28624763 | A | G | 0.0301 | −0.0245 | 0.9758 | 0.9985 |
| | A | | | | | | | | | |
| 12 | 12_125674073_T_T | 125674073 | 125189527 | | TG | T | 0.3895 | −0.0011 | 0.9989 | 0.9991 |
| | G | | | | | | | | | |
| 12 | 12_127144291_A_ | 127144291 | 126659745 | rs7138452 | G | A | 0.7640 | 0.0064 | 1.0064 | 1.0098 |
| | G | | | | | | | | | |
| 12 | 12_129209173_T_ | 129209173 | 128724628 | rs61669394 | G | T | 0.0181 | 0.0280 | 1.0284 | 1.0010 |
| | G | | | | | | | | | |
| 12 | 12_130476989_C_ | 130476989 | 129992444 | rs117236628 | G | C | 0.0200 | −0.0223 | 0.9779 | 0.9991 |
| | G | | | | | | | | | |
| 13 | 13_19536600_A_C | 19536600 | 18962460 | rs11617737 | C | A | 0.4332 | −0.0025 | 0.9975 | 0.9978 |
| 13 | 13_21019209_C_G | 21019209 | 20445070 | rs73160875 | G | C | 0.0155 | 0.0338 | 1.0344 | 1.0011 |
| 13 | 13_23625664_G_A | 23625664 | 23051525 | rs7986188 | A | G | 0.6557 | 0.0010 | 1.0010 | 1.0013 |
| 13 | 13_24281011_T_C | 24281011 | 23706872 | rs4770478 | C | T | 0.1748 | 0.0006 | 1.0006 | 1.0002 |
| 13 | 13_24298749_CA_ | 24298749 | 23724610 | 13:24298749_ | C | CA | 0.1624 | 0.0001 | 1.0001 | 1.0000 |
| | C | | | CA_C | | | | | | |
| 13 | 13_24341843_G_A | 24341843 | 23767704 | rs9510851 | A | G | 0.1703 | 0.0112 | 1.0113 | 1.0038 |
| 13 | 13_24971857_C_T | 24971857 | 24397719 | rs55813922 | T | C | 0.1334 | 0.0123 | 1.0124 | 1.0033 |
| 13 | 13_24976086_T_C | 24976086 | 24401948 | rs7321198 | C | T | 0.5663 | 0.0028 | 1.0028 | 1.0032 |
| 13 | 13_24984540_AC_T | 24984540 | 24410402 | | TC | AC | 0.3572 | −0.0046 | 0.9954 | 0.9967 |
| | C | | | | | | | | | |
| 13 | 13_25018249_C_T | 25018249 | 24444111 | rs143552353 | T | C | 0.0339 | −0.0020 | 0.9980 | 0.9999 |
| 13 | 13_30107388_T_C | 30107388 | 29533251 | rs3783256 | C | T | 0.6418 | −0.0062 | 0.9938 | 0.9921 |
| 13 | 13_30118193_C_T | 30118193 | 29544056 | rs9578107 | T | C | 0.0971 | 0.0095 | 1.0095 | 1.0019 |
| 13 | 13_32704954_AT_ | 32704954 | 32130817 | 13:32704954_ | A | AT | 0.3699 | 0.0029 | 1.0029 | 1.0021 |
| | A | | | AT_A | | | | | | |
| 13 | 13_32755706_C_T | 32755706 | 32181569 | rs2520691 | T | C | 0.1984 | −0.0017 | 0.9983 | 0.9993 |
| 13 | 13_32773204_C_T | 32773204 | 32199067 | rs73169113 | T | C | 0.1829 | 0.0125 | 1.0126 | 1.0046 |
| 13 | 13_32798235_T_TA | 32798235 | 32224098 | rs5802638 | TA | T | 0.5514 | 0.0017 | 1.0017 | 1.0019 |
| 13 | 13_32972626_A_T | 32972626 | 32398489 | rs11571833 | T | A | 0.0079 | 0.1809 | 1.1983 | 1.0031 |
| 13 | 13_33170934_A_G | 33170934 | 32596797 | rs9535012 | G | A | 0.1065 | 0.0000 | 1.0000 | 1.0000 |
| 13 | 13_34752214_T_A | 34752214 | 34178077 | rs9598722 | A | T | 0.4438 | −0.0026 | 0.9974 | 0.9977 |
| 13 | 13_34784728_AT_ | 34784728 | 34210591 | 13:34784728_ | A | AT | 0.4373 | 0.0074 | 1.0074 | 1.0065 |
| | A | | | AT_A | | | | | | |
| 13 | 13_36071786_T_C | 36071786 | 35497649 | rs1381476 | C | T | 0.6499 | −0.0100 | 0.9900 | 0.9871 |
| 13 | 13_36072172_G_A | 36072172 | 35498035 | rs1466402 | A | G | 0.9553 | −0.0140 | 0.9861 | 0.9736 |
| 13 | 13_36133143_T_A | 36133143 | 35559006 | rs73171571 | A | T | 0.0240 | 0.0029 | 1.0029 | 1.0001 |
| 13 | 13_36521433_T_G | 36521433 | 35947296 | rs1936004 | G | T | 0.6645 | −0.0029 | 0.9971 | 0.9962 |
| 13 | 13_37897575_G_A | 37897575 | 37323438 | rs2209245 | A | G | 0.8802 | −0.0004 | 0.9996 | 0.9993 |
| 13 | 13_39337618_C_G | 39337618 | 38763481 | rs55864343 | G | C | 0.7249 | 0.0065 | 1.0065 | 1.0095 |
| 13 | 13_40088007_G_A | 40088007 | 39513870 | rs7981122 | A | G | 0.2279 | 0.0030 | 1.0030 | 1.0014 |
| 13 | 13_40339158_C_T | 40339158 | 39765021 | rs73179574 | T | C | 0.0674 | 0.0006 | 1.0006 | 1.0001 |
| 13 | 13_40355430_G_A | 40355430 | 39781293 | rs73179582 | A | G | 0.0529 | 0.0144 | 1.0145 | 1.0015 |
| 13 | 13_40658787_C_T | 40658787 | 40084650 | rs1414932 | T | C | 0.5009 | 0.0071 | 1.0071 | 1.0072 |
| 13 | 13_40772172_T_C | 40772172 | 40198035 | rs61954176 | C | T | 0.2187 | 0.0024 | 1.0024 | 1.0011 |
| 13 | 13_40808620_G_A | 40808620 | 40234483 | rs4943745 | A | G | 0.7499 | −0.0030 | 0.9970 | 0.9955 |
| 13 | 13_40815320_G_A | 40815320 | 40241183 | rs11147786 | A | G | 0.0647 | 0.0062 | 1.0062 | 1.0008 |
| 13 | 13_40824180_C_T | 40824180 | 40250043 | rs11840120 | T | C | 0.1530 | 0.0095 | 1.0095 | 1.0029 |
| 13 | 13_41891170_C_T | 41891170 | 41317034 | rs7319540 | T | C | 0.0742 | −0.0117 | 0.9884 | 0.9983 |
| 13 | 13_41981801_AT_ | 41981801 | 41407665 | 13:4198180_1 | A | AT | 0.0657 | −0.0001 | 0.9999 | 1.0000 |
| | A | | | AT_A | | | | | | |
| 13 | 13_41988053_C_A | 41988053 | 41413917 | rs4427698 | A | C | 0.1760 | −0.0096 | 0.9904 | 0.9966 |
| 13 | 13_43501356_A_G | 43501356 | 42927220 | rs9315973 | G | A | 0.8303 | 0.0251 | 1.0254 | 1.0427 |
| 13 | 13_43522038_G_A | 43522038 | 42947902 | rs12427475 | A | G | 0.0608 | 0.0000 | 1.0000 | 1.0000 |
| 13 | 13_48352745_G_A | 48352745 | 47778610 | rs149670665 | A | G | 0.0200 | −0.0007 | 0.9993 | 1.0000 |
| 13 | 13_51085041_G_A | 51085041 | 50510905 | rs9562985 | A | G | 0.2441 | −0.0095 | 0.9905 | 0.9954 |
| 13 | 13_57924666_G_C | 57924666 | 57350532 | rs78449128 | C | G | 0.0180 | −0.0272 | 0.9732 | 0.9990 |
| 13 | 13_57984399_G_A | 57984399 | 57410265 | rs78263459 | A | G | 0.0322 | −0.0263 | 0.9740 | 0.9983 |
| 13 | 13_58257265_A_G | 58257265 | 57683131 | rs9597587 | G | A | 0.2958 | −0.0062 | 0.9938 | 0.9963 |
| 13 | 13_58463779_C_T | 58463779 | 57889645 | rs34667220 | T | C | 0.2481 | 0.0073 | 1.0073 | 1.0036 |
| 13 | 13_58713360_C_G | 58713360 | 58139226 | rs3012850 | G | C | 0.2380 | 0.0028 | 1.0028 | 1.0013 |
| 13 | 13_61217736_C_T | 61217736 | 60643602 | rs61968726 | T | C | 0.0124 | 0.0227 | 1.0230 | 1.0006 |
| 13 | 13_69370155_T_G | 69370155 | 68796023 | rs117211244 | G | T | 0.0443 | 0.0059 | 1.0059 | 1.0005 |
| 13 | 13_70007016_C_T | 70007016 | 69432884 | rs353836 | T | C | 0.5833 | 0.0000 | 1.0000 | 1.0000 |
| 13 | 13_70205508_T_G | 70205508 | 69631376 | rs117950521 | G | T | 0.0528 | 0.0000 | 1.0000 | 1.0000 |
| 13 | 13_73806982_T_C | 73806982 | 73232845 | rs12870942 | C | T | 0.3153 | 0.0108 | 1.0109 | 1.0069 |
| 13 | 13_73818924_A_A | 73818924 | 73244787 | rs111683019 | AT | A | 0.4544 | 0.0000 | 1.0000 | 1.0000 |
| | T | | | | | | | | | |
| 13 | 13_73960952_A_G | 73960952 | 73386815 | rs2181965 | G | A | 0.7618 | 0.0138 | 1.0139 | 1.0213 |
| 13 | 13_73967507_T_C | 73967507 | 73393370 | rs4885096 | C | T | 0.6870 | 0.0081 | 1.0081 | 1.0112 |
| 13 | 13_78996578_C_T | 78996578 | 78422443 | rs61964767 | T | C | 0.0313 | −0.0164 | 0.9837 | 0.9990 |
| 13 | 13_80391295_A_C | 80391295 | 79817160 | rs4386023 | C | A | 0.4948 | −0.0030 | 0.9970 | 0.9970 |
| 13 | 13_85861553_T_G | 85861553 | 85287418 | rs9566061 | G | T | 0.0353 | −0.0053 | 0.9947 | 0.9996 |
| 13 | 13_88381395_C_T | 88381395 | 87729140 | rs117480076 | T | C | 0.0188 | −0.0018 | 0.9982 | 0.9999 |
| 13 | 13_90611517_A_G | 90611517 | 89959263 | rs12050059 | G | A | 0.2091 | −0.0187 | 0.9815 | 0.9923 |
| 13 | 13_91434105_G_C | 91434105 | 90781851 | rs56375900 | C | G | 0.1741 | 0.0064 | 1.0064 | 1.0022 |
| 13 | 13_91939972_CAA | 91939972 | 91287718 | rs375709528 | C | CAAA | 0.4593 | −0.0103 | 0.9898 | 0.9906 |
| | AT_C | | | | | T | | | | |
| 13 | 13_92457036_T_C | 92457036 | 91804782 | rs1854971 | C | T | 0.5565 | 0.0110 | 1.0111 | 1.0123 |
| 13 | 13_92792534_C_T | 92792534 | 92140281 | rs17188390 | T | C | 0.1587 | −0.0065 | 0.9935 | 0.9979 |
| 13 | 13_93663234_T_C | 93663234 | 93010981 | rs76619105 | C | T | 0.0459 | −0.0012 | 0.9988 | 0.9999 |
| 13 | 13_93897052_T_G | 93897052 | 93244799 | rs60426741 | G | T | 0.0754 | −0.0097 | 0.9903 | 0.9985 |
| 13 | 13_97886728_T_C | 97886728 | 97234474 | rs9516878 | C | T | 0.7067 | 0.0036 | 1.0036 | 1.0051 |
| 13 | 13_98815232_C_T | 98815232 | 98162978 | rs72653394 | T | C | 0.0666 | 0.0156 | 1.0157 | 1.0021 |
| 13 | 13_99068255_T_C | 99068255 | 98416001 | rs12429460 | C | T | 0.1370 | −0.0044 | 0.9956 | 0.9988 |
| 13 | 13_99429807_C_T | 99429807 | 98777553 | rs9557057 | T | C | 0.3229 | −0.0037 | 0.9963 | 0.9976 |
| 13 | 13_100648099_A_ | 100648099 | 99995845 | rs1347190 | G | A | 0.4435 | −0.0035 | 0.9965 | 0.9969 |
| | G | | | | | | | | | |
| 13 | 13_100693650_TA_ | 100693650 | 100041396 | | T | TA | 0.5201 | −0.0089 | 0.9911 | 0.9908 |
| | T | | | | | | | | | |
| 13 | 13_103141574_G_ | 103141574 | 102489224 | rs10508088 | A | G | 0.0528 | −0.0030 | 0.9970 | 0.9997 |
| | A | | | | | | | | | |
| 13 | 13_103155752_A_C | 103155752 | 102503402 | rs78529221 | C | A | 0.0752 | −0.0024 | 0.9976 | 0.9996 |
| 13 | 13_103424321_T_C | 103424321 | 102771971 | rs151250473 | C | T | 0.0131 | −0.0137 | 0.9864 | 0.9996 |
| 13 | 13_104277179_A_ | 104277179 | 103624829 | rs7320001 | G | A | 0.0473 | 0.0032 | 1.0032 | 1.0003 |
| | G | | | | | | | | | |
| 13 | 13_105115053_C_A | 105115053 | 104462703 | rs73594938 | A | C | 0.0154 | −0.0407 | 0.9601 | 0.9988 |
| 13 | 13_106422213_G_ | 106422213 | 105769864 | rs1576158 | A | G | 0.6553 | 0.0041 | 1.0041 | 1.0054 |
| | A | | | | | | | | | |
| 13 | 13_107577537_A_ | 107577537 | 106925189 | rs9555296 | G | A | 0.2937 | 0.0064 | 1.0064 | 1.0038 |
| | G | | | | | | | | | |
| 13 | 13_107680606_G_ | 107680606 | 107028258 | rs12877195 | A | G | 0.0577 | −0.0006 | 0.9994 | 0.9999 |
| | A | | | | | | | | | |
| 13 | 13_108669929_A_ | 108669929 | 108017581 | rs12875908 | G | A | 0.5196 | 0.0030 | 1.0030 | 1.0031 |
| | G | | | | | | | | | |
| 13 | 13_110629518_C_ | 110629518 | 109977171 | rs7334266 | G | C | 0.2438 | 0.0096 | 1.0096 | 1.0047 |
| | G | | | | | | | | | |
| 13 | 13_110651131_C_T | 110651131 | 109998784 | rs11069818 | T | C | 0.4934 | 0.0008 | 1.0008 | 1.0008 |
| 13 | 13_110857823_C_T | 110857823 | 110205476 | rs482757 | T | C | 0.6356 | 0.0124 | 1.0125 | 1.0159 |
| 13 | 13_111592904_G_ | 111592904 | 110940557 | rs60926826 | A | G | 0.1410 | −0.0006 | 0.9994 | 0.9998 |
| | A | | | | | | | | | |
| 13 | 13_111649724_T_C | 111649724 | 110997377 | rs61968960 | C | T | 0.1654 | −0.0072 | 0.9928 | 0.9976 |
| 13 | 13_112077465_C | 112077465 | 111425118 | rs2528773 | G | C | 0.5521 | 0.0074 | 1.0074 | 1.0082 |
| | G | | | | | | | | | |
| 13 | 13_114323997_T_C | 114323997 | 113669682 | rs9796234 | C | T | 0.5006 | 0.0072 | 1.0072 | 1.0072 |
| 13 | 13_114433140_G_ | 114433140 | 113730167 | rs36149740 | A | G | 0.4614 | −0.0001 | 0.9999 | 0.9999 |
| | A | | | | | | | | | |
| 13 | 13_114632156_T_C | 114632156 | 113929183 | rs9604487 | C | T | 0.0450 | −0.0146 | 0.9855 | 0.9987 |
| 14 | 14_23317825_A_G | 23317825 | 22848616 | rs17882077 | G | A | 0.3725 | 0.0070 | 1.0070 | 1.0052 |
| 14 | 14_24730100_A_C | 24730100 | 24260894 | rs7151201 | C | A | 0.1879 | 0.0032 | 1.0032 | 1.0012 |
| 14 | 14_24735624_A_G | 24735624 | 24266418 | rs2877612 | G | A | 0.2328 | 0.0065 | 1.0065 | 1.0030 |
| 14 | 14_24736027_G_A | 24736027 | 24266821 | rs14193 | A | G | 0.3619 | 0.0025 | 1.0025 | 1.0018 |
| 14 | 14_25555593_C_T | 25555593 | 25086387 | rs4494455 | T | C | 0.3012 | −0.0076 | 0.9924 | 0.9954 |
| 14 | 14_25630912_G_A | 25630912 | 25161706 | rs17109642 | A | G | 0.1904 | 0.0100 | 1.0101 | 1.0038 |
| 14 | 14_25655764_A_G | 25655764 | 25186558 | rs11159142 | G | A | 0.6991 | 0.0036 | 1.0036 | 1.0050 |
| 14 | 14_25695907_A_G | 25695907 | 25226701 | rs17212065 | G | A | 0.1092 | 0.0055 | 1.0055 | 1.0012 |
| 14 | 14_25875224_T_G | 25875224 | 25406018 | rs72688549 | G | T | 0.1497 | 0.0040 | 1.0040 | 1.0012 |
| 14 | 14_27865166_A_G | 27865166 | 27395960 | rs8021545 | G | A | 0.0912 | −0.0160 | 0.9841 | 0.9971 |
| 14 | 14_32012593_G_A | 32012593 | 31543387 | rs8016747 | A | G | 0.7366 | −0.0095 | 0.9905 | 0.9861 |
| 14 | 14_32310803_T_C | 32310803 | 31841597 | rs7158089 | C | T | 0.3163 | 0.0048 | 1.0048 | 1.0030 |
| 14 | 14_32596649_T_C | 32596649 | 32127443 | rs140783387 | C | T | 0.0887 | 0.0230 | 1.0233 | 1.0041 |
| 14 | 14_32900776_C_A | 32900776 | 32431570 | rs71419908 | A | C | 0.4487 | 0.0020 | 1.0020 | 1.0018 |
| 14 | 14_32987758_A_G | 32987758 | 32518552 | rs148987100 | G | A | 0.2279 | −0.0128 | 0.9873 | 0.9942 |
| 14 | 14_33046471_T_G | 33046471 | 32577265 | rs768787 | G | T | 0.6510 | 0.0081 | 1.0081 | 1.0106 |
| 14 | 14_35260354_A_A | 35260354 | 34791148 | | AAAGAG | A | 0.1359 | 0.0000 | 1.0000 | 1.0000 |
| | AAGAG | | | | | | | | | |
| 14 | 14_35323160_T_C | 35323160 | 34853954 | rs10129577 | C | T | 0.1015 | −0.0076 | 0.9924 | 0.9985 |
| 14 | 14_35359783_C_T | 35359783 | 34890577 | rs59307778 | T | C | 0.3970 | 0.0062 | 1.0062 | 1.0049 |
| 14 | 14_35412105_G_G | 35412105 | 34942899 | rs542632225 | GA | G | 0.3003 | −0.0039 | 0.9961 | 0.9977 |
| | A | | | | | | | | | |
| 14 | 14_35422811_C_T | 35422811 | 34953605 | rs75759052 | T | C | 0.2217 | −0.0015 | 0.9985 | 0.9993 |
| 14 | 14_35582138_A_G | 35582138 | 35112932 | rs12879871 | G | A | 0.3869 | −0.0103 | 0.9898 | 0.9921 |
| 14 | 14_37122790_A_G | 37122790 | 36653585 | rs1018464 | G | A | 0.2478 | −0.0069 | 0.9931 | 0.9966 |
| 14 | 14_37123250_A_C | 37123250 | 36654045 | rs1018466 | C | A | 0.5321 | 0.0068 | 0.9932 | 0.9928 |
| 14 | 14_37128564_C_A | 37128564 | 36659359 | rs34914085 | A | C | 0.2122 | −0.0356 | 0.9650 | 0.9852 |
| 14 | 14_37129341_A_C | 37129341 | 36660136 | rs2073242 | C | A | 0.6189 | 0.0000 | 1.0000 | 1.0000 |
| 14 | 14_37228504_C_T | 37228504 | 36759299 | rs2253012 | T | C | 0.4434 | 0.0152 | 1.0153 | 1.0136 |
| 14 | 14_37268797_G_A | 37268797 | 36799592 | rs848092 | A | G | 0.3396 | −0.0035 | 0.9965 | 0.9976 |
| 14 | 14_37481041_G_A | 37481041 | 37011836 | rs17767511 | A | G | 0.1012 | −0.0074 | 0.9926 | 0.9985 |
| 14 | 14_37660202_G_A | 37660202 | 37190997 | rs117137044 | A | G | 0.1075 | −0.0128 | 0.9873 | 0.9973 |
| 14 | 14_38142026_ATT_ | 38142026 | 37672821 | rs774465251 | A | ATT | 0.2735 | 0.0104 | 1.0105 | 1.0057 |
| | A | | | | | | | | | |
| 14 | 14_38208687_G_C | 38208687 | 37739482 | rs2415410 | C | G | 0.6884 | −0.0030 | 0.9970 | 0.9959 |
| 14 | 14_38848824_T_A | 38848824 | 38379620 | rs1168548 | A | T | 0.2795 | −0.0003 | 0.9997 | 0.9998 |
| 14 | 14_38849500_A_G | 38849500 | 38380296 | rs8023173 | G | A | 0.3203 | 0.0000 | 1.0000 | 1.0000 |
| 14 | 14_38996181_TCAT | 38996181 | 38526977 | 14:38996181_ | T | TCAT | 0.0239 | −0.0113 | 0.9888 | 0.9995 |
| | TTAAAGCCTTC_T | | | TCATTTAAAGC | | TTAA | | | | |
| | | | | CTTC_T | | AGCC | | | | |
| | | | | | | TTC | | | | |
| 14 | 14_38996493_G_A | 38996493 | 38527289 | rs75951442 | A | G | 0.0353 | −0.0002 | 0.9998 | 1.0000 |
| 14 | 14_39414930_C_T | 39414930 | 38945726 | rs1951221 | T | C | 0.8803 | −0.0114 | 0.9887 | 0.9801 |
| 14 | 14_39416898_C_T | 39416898 | 38947694 | rs2025063 | T | C | 0.5403 | −0.0024 | 0.9976 | 0.9974 |
| 14 | 14_39674203_C_A | 39674203 | 39204999 | rs10149495 | A | C | 0.1213 | −0.0015 | 0.9985 | 0.9996 |
| 14 | 14_40925291_G_A | 40925291 | 40456087 | rs78979382 | A | G | 0.0369 | 0.0137 | 1.0138 | 1.0010 |
| 14 | 14_40955886_CT_C | 40955886 | 40486682 | 14:40955886 | C | CT | 0.0369 | 0.0170 | 1.0171 | 1.0013 |
| | | | | CT_C | | | | | | |
| 14 | 14_42183290_AT_ | 42183290 | 41714087 | 14:42183290_ | A | AT | 0.0243 | −0.0270 | 0.9734 | 0.9987 |
| | A | | | AT_A | | | | | | |
| 14 | 14_45043928_C_T | 45043928 | 44574725 | rs10152023 | T | C | 0.5769 | −0.0069 | 0.9931 | 0.9921 |
| 14 | 14_45397355_A_C | 45397355 | 44928152 | rs79851852 | C | A | 0.0211 | 0.0000 | 1.0000 | 1.0000 |
| 14 | 14_49274078_C_G | 49274078 | 48804875 | rs2416185 | G | C | 0.0825 | 0.0000 | 1.0000 | 1.0000 |
| 14 | 14_50520602_C_T | 50520602 | 50053884 | rs7156043 | T | C | 0.1140 | −0.0086 | 0.9914 | 0.9980 |
| 14 | 14_52123148_C_T | 52123148 | 51656430 | rs1966185 | T | C | 0.9492 | 0.0204 | 1.0206 | 1.0395 |
| 14 | 14_52282868_CGG | 52282868 | 51816150 | rs771414926 | C | CGGT | 0.5632 | −0.0034 | 0.9966 | 0.9962 |
| | TGAGCGT_C | | | | | GAGC | | | | |
| | | | | | | GT | | | | |
| 14 | 14_53696414_A_T | 53696414 | 53229696 | rs7401132 | T | A | 0.2721 | −0.0088 | 0.9912 | 0.9952 |
| 14 | 14_53711660_T_C | 53711660 | 53244942 | rs12891259 | C | T | 0.1043 | 0.0059 | 1.0059 | 1.0012 |
| 14 | 14_53890820_T_C | 53890820 | 53424102 | rs7151904 | C | T | 0.8285 | 0.0011 | 1.0011 | 1.0018 |
| 14 | 14_54017042_G_A | 54017042 | 53550324 | rs79974315 | A | G | 0.0609 | 0.0196 | 1.0198 | 1.0024 |
| 14 | 14_58810321_C_T | 58810321 | 58343603 | rs61974494 | T | C | 0.0206 | 0.0000 | 1.0000 | 1.0000 |
| 14 | 14_59100519_C_C | 59100519 | 58633801 | rs529583772 | CAA | C | 0.6868 | −0.0071 | 0.9929 | 0.9903 |
| | AA | | | | | | | | | |
| 14 | 14_59108678_T_C | 59108678 | 58641960 | rs167481 | C | T | 0.7415 | −0.0021 | 0.9979 | 0.9969 |
| 14 | 14_59121567_CA_ | 59121567 | 58654849 | rs569494089 | C | CA | 0.3847 | −0.0009 | 0.9991 | 0.9993 |
| | C | | | | | | | | | |
| 14 | 14_60505617_G_T | 60505617 | 60038899 | rs192657698 | T | G | 0.0172 | −0.0127 | 0.9874 | 0.9996 |
| 14 | 14_62389498_T_C | 62389498 | 61922780 | rs150761741 | C | T | 0.0120 | 0.0051 | 1.0051 | 1.0001 |
| 14 | 14_62588317_G_A | 62588317 | 62121599 | rs74506686 | A | G | 0.1656 | 0.0161 | 1.0162 | 1.0054 |
| 14 | 14_63173004_C_G | 63173004 | 62706286 | rs1889370 | G | C | 0.5119 | 0.0015 | 1.0015 | 1.0015 |
| 14 | 14_63177144_T_C | 63177144 | 62710426 | rs869834 | C | T | 0.4175 | −0.0024 | 0.9976 | 0.9980 |
| 14 | 14_63181859_A_C | 63181859 | 62715141 | rs1027171 | C | A | 0.4284 | 0.0069 | 1.0069 | 1.0059 |
| 14 | 14_63207288_T_C | 63207288 | 62740570 | rs10133621 | C | T | 0.5549 | 0.0021 | 1.0021 | 1.0023 |
| 14 | 14_63681322_C_T | 63681322 | 63214604 | rs4899110 | T | C | 0.5989 | 0.0000 | 1.0000 | 1.0000 |
| 14 | 14_65655124_G_A | 65655124 | 65188406 | rs55839896 | A | G | 0.1735 | −0.0088 | 0.9912 | 0.9970 |
| 14 | 14_68423215_G_A | 68423215 | 67956498 | rs28649231 | A | G | 0.1932 | 0.0000 | 1.0000 | 1.0000 |
| 14 | 14_68583829_A_C | 68583829 | 68117112 | rs378408 | C | A | 0.1749 | 0.0000 | 1.0000 | 1.0000 |
| 14 | 14_68589677_A_A | 68589677 | 68122960 | rs145099242 | AAC | A | 0.1583 | 0.0000 | 1.0000 | 1.0000 |
| | AC | | | | | | | | | |
| 14 | 14_68650809_CAA | 68650809 | 68184092 | 14:68650809_ | C | CAA | 0.8293 | −0.0130 | 0.9871 | 0.9787 |
| | C | | | CAA_C | | | | | | |
| 14 | 14_68660428_T_C | 68660428 | 68193711 | rs2588809 | C | T | 0.8345 | −0.0063 | 0.9937 | 0.9895 |
| 14 | 14_68723484_C_G | 68723484 | 68256767 | rs3784095 | G | C | 0.2487 | 0.0000 | 1.0000 | 1.0000 |
| 14 | 14_68968551_C_G | 68968551 | 68501834 | rs2525511 | G | C | 0.6754 | −0.0006 | 0.9994 | 0.9992 |
| 14 | 14_68974410_A_G | 68974410 | 68507693 | rs739874 | G | A | 0.2631 | −0.0138 | 0.9863 | 0.9928 |
| 14 | 14_68975374_C_T | 68975374 | 68508657 | rs2842346 | T | C | 0.2412 | 0.0000 | 1.0000 | 1.0000 |
| 14 | 14_68979835_T_C | 68979835 | 68513118 | rs11624333 | C | T | 0.2581 | −0.0272 | 0.9732 | 0.9862 |
| 14 | 14_69005614_G_A | 69005614 | 68538897 | rs34636650 | A | G | 0.2628 | −0.0106 | 0.9895 | 0.9945 |
| 14 | 14_69026379_G_A | 69026379 | 68559662 | rs11844632 | A | G | 0.2728 | −0.0118 | 0.9883 | 0.9936 |
| 14 | 14_69035425_G_A | 69035425 | 68568708 | rs8007194 | A | G | 0.4698 | −0.0034 | 0.9966 | 0.9968 |
| 14 | 14_69047006_C_T | 69047006 | 68580289 | rs74933543 | T | C | 0.2968 | −0.0098 | 0.9902 | 0.9942 |
| 14 | 14_69098816_G_T | 69098816 | 68632099 | rs7153037 | T | G | 0.1531 | 0.0026 | 1.0026 | 1.0008 |
| 14 | 14_69114465_T_G | 69114465 | 68647748 | rs61987115 | G | T | 0.1330 | −0.0042 | 0.9958 | 0.9989 |
| 14 | 14_69312861_T_C | 69312861 | 68846144 | rs8012068 | C | T | 0.0910 | −0.0107 | 0.9894 | 0.9981 |
| 14 | 14_69314579_A_T | 69314579 | 68847862 | rs79514844 | T | A | 0.6310 | 0.0020 | 1.0020 | 1.0025 |
| 14 | 14_69449755_A_C | 69449755 | 68983038 | rs78009683 | C | A | 0.2295 | −0.0079 | 0.9921 | 0.9964 |
| 14 | 14_73281244_G_C | 73281244 | 72814536 | rs4479167 | C | G | 0.9397 | 0.0009 | 1.0009 | 1.0017 |
| 14 | 14_76784399_T_G | 76784399 | 76318056 | rs34966488 | G | T | 0.6594 | −0.0060 | 0.9940 | 0.9921 |
| 14 | 14_76803463_G_A | 76803463 | 76337120 | rs11623829 | A | G | 0.3982 | −0.0001 | 0.9999 | 0.9999 |
| 14 | 14_77130070_A_G | 77130070 | 76663727 | rs4903445 | G | A | 0.1640 | −0.0108 | 0.9893 | 0.9965 |
| 14 | 14_77474707_C_T | 77474707 | 77008364 | rs1558858 | T | C | 0.9828 | −0.0246 | 0.9757 | 0.9528 |
| 14 | 14_77545648_T_C | 77545648 | 77079305 | rs12897324 | C | T | 0.5692 | −0.0006 | 0.9994 | 0.9993 |
| 14 | 14_77547212_C_T | 77547212 | 77080869 | rs10873302 | T | C | 0.2834 | −0.0081 | 0.9919 | 0.9954 |
| 14 | 14_77733856_C_G | 77733856 | 77267513 | rs28988624 | G | C | 0.0241 | −0.0343 | 0.9663 | 0.9984 |
| 14 | 14_78445003_G_C | 78445003 | 77978660 | rs11625014 | C | G | 0.7618 | −0.0059 | 0.9941 | 0.9911 |
| 14 | 14_78506992_A_G | 78506992 | 78040649 | rs1955577 | G | A | 0.6700 | −0.0024 | 0.9976 | 0.9968 |
| 14 | 14_81488416_C_T | 81488416 | 81022072 | rs35789100 | T | C | 0.1163 | −0.0006 | 0.9994 | 0.9999 |
| 14 | 14_81743718_C_T | 81743718 | 81277374 | rs34323725 | T | C | 0.1042 | 0.0087 | 1.0087 | 1.0018 |
| 14 | 14_83131247_C_T | 83131247 | 82664903 | rs17616180 | T | C | 0.3169 | −0.0040 | 0.9960 | 0.9975 |
| 14 | 14_83187970_A_C | 83187970 | 82721626 | rs1370761 | C | A | 0.2330 | −0.0150 | 0.9851 | 0.9931 |
| 14 | 14_84156160_C_T | 84156160 | 83689816 | rs116988070 | T | C | 0.0550 | −0.0027 | 0.9973 | 0.9997 |
| 14 | 14_84253506_A_G | 84253506 | 83787162 | rs76569814 | G | A | 0.2805 | −0.0052 | 0.9948 | 0.9971 |
| 14 | 14_84262197_T_A | 84262197 | 83795853 | rs12895560 | A | T | 0.3417 | −0.0004 | 0.9996 | 0.9997 |
| 14 | 14_84262786_T_A | 84262786 | 83796442 | rs8021562 | A | T | 0.4746 | −0.0017 | 0.9983 | 0.9984 |
| 14 | 14_84938169_C_T | 84938169 | 84471825 | rs72687274 | T | C | 0.0444 | −0.0081 | 0.9919 | 0.9993 |
| 14 | 14_85106220_CTCC | 85106220 | 84639876 | rs138543705 | GTCCCAT | CTCC | 0.0159 | −0.0498 | 0.9514 | 0.9985 |
| | CAT_GTCCCAT | | | | | CAT | | | | |
| 14 | 14_85678604_C_T | 85678604 | 85212260 | rs4467007 | T | C | 0.5580 | 0.0054 | 1.0054 | 1.0061 |
| 14 | 14_85682017_G_T | 85682017 | 85215673 | rs1667521 | T | G | 0.7368 | 0.0042 | 1.0042 | 1.0062 |
| 14 | 14_88180858_C_T | 88180858 | 87714514 | rs1959718 | T | C | 0.9349 | 0.0066 | 1.0066 | 1.0124 |
| 14 | 14_89885100_CCT_ | 89885100 | 89418756 | 14:89885100_ | C | CCT | 0.1574 | −0.0118 | 0.9883 | 0.9963 |
| | C | | | CCT_C | | | | | | |
| 14 | 14_91574779_G_A | 91574779 | 91108435 | rs140850061 | A | G | 0.0187 | 0.0007 | 1.0007 | 1.0000 |
| 14 | 14_91734164_T_G | 91734164 | 91267820 | rs12895175 | G | T | 0.2424 | 0.0000 | 1.0000 | 1.0000 |
| 14 | 14_91751788_TC_T | 91751788 | 91285444 | 14:91751788_ | T | TC | 0.6934 | 0.0212 | 1.0214 | 1.0299 |
| | | | | TC_T | | | | | | |
| 14 | 14_91757542_T_C | 91757542 | 91291198 | rs3825658 | C | T | 0.1193 | 0.0000 | 1.0000 | 1.0000 |
| 14 | 14_91841069_A_G | 91841069 | 91374725 | rs941764 | G | A | 0.3444 | 0.0214 | 1.0216 | 1.0150 |
| 14 | 14_91845133_A_G | 91845133 | 91378789 | rs1046311 | G | A | 0.1781 | 0.0072 | 1.0072 | 1.0026 |
| 14 | 14_91848177_A_A | 91848177 | 91381833 | rs113889692 | AAAC | A | 0.7571 | −0.0002 | 0.9998 | 0.9997 |
| | AAC | | | | | | | | | |
| 14 | 14_91880062_G_A | 91880062 | 91413718 | rs17804398 | A | G | 0.2452 | −0.0007 | 0.9993 | 0.9997 |
| 14 | 14_91888924_T_TA | 91888924 | 91422580 | rs150658557 | TA | T | 0.1986 | −0.0083 | 0.9917 | 0.9967 |
| 14 | 14_92011796_TTTT | 92011796 | 91545452 | 14:92011796_ | T | TTTT | 0.2549 | −0.0040 | 0.9960 | 0.9980 |
| | C_T | | | TTTTC_T | | C | | | | |
| 14 | 14_92032916_C_T | 92032916 | 91566572 | rs117534345 | T | C | 0.0134 | −0.0274 | 0.9730 | 0.9993 |
| 14 | 14_92996181_G_A | 92996181 | 92529837 | rs8019318 | A | G | 0.4094 | 0.0102 | 1.0103 | 1.0084 |
| 14 | 14_93004912_G_A | 93004912 | 92538568 | rs8015844 | A | G | 0.4104 | 0.0026 | 1.0026 | 1.0021 |
| 14 | 14_93005020_C_G | 93005020 | 92538676 | rs74645156 | G | C | 0.1513 | −0.0105 | 0.9896 | 0.9968 |
| 14 | 14_93035214_T_C | 93035214 | 92568869 | rs2104240 | C | T | 0.0658 | 0.0042 | 1.0042 | 1.0006 |
| 14 | 14_93070286_C_T | 93070286 | 92603941 | rs78440108 | T | C | 0.1709 | −0.0004 | 0.9996 | 0.9999 |
| 14 | 14_93078646_CAG | 93078646 | 92612301 | rs758966327 | C | CAG | 0.2563 | −0.0058 | 0.9942 | 0.9970 |
| | _C | | | | | | | | | |
| 14 | 14_93104072_T_C | 93104072 | 92637727 | rs11627032 | C | T | 0.2530 | −0.0147 | 0.9854 | 0.9926 |
| 14 | 14_93132299_G_C | 93132299 | 92665954 | rs12895078 | C | G | 0.4609 | −0.0038 | 0.9962 | 0.9965 |
| 14 | 14_93232421_T_A | 93232421 | 92766076 | rs144169808 | A | T | 0.1589 | 0.0118 | 1.0119 | 1.0038 |
| 14 | 14_93263033_C_T | 93263033 | 92796688 | rs12437409 | T | C | 0.5827 | 0.0016 | 1.0016 | 1.0019 |
| 14 | 14_95789794_T_C | 95789794 | 95323457 | rs68179258 | C | T | 0.2397 | 0.0106 | 1.0107 | 1.0051 |
| 14 | 14_99690907_C_A | 99690907 | 99224570 | rs807736 | A | C | 0.8433 | 0.0048 | 1.0048 | 1.0081 |
| 14 | 14_100257281_A_ | 100257281 | 99790944 | rs7154519 | G | A | 0.3971 | 0.0019 | 1.0019 | 1.0015 |
| | G | | | | | | | | | |
| 14 | 14_101303361_C_T | 101303361 | 100837024 | rs8019930 | T | C | 0.3692 | −0.0079 | 0.9921 | 0.9942 |
| 14 | 14_101311729_C_T | 101311729 | 100845392 | rs56363527 | T | C | 0.3268 | −0.0031 | 0.9969 | 0.9980 |
| 14 | 14_101318311_C_ | 101318311 | 100851974 | rs2295389 | G | C | 0.3639 | −0.0025 | 0.9975 | 0.9982 |
| | G | | | | | | | | | |
| 14 | 14_102628910_TTC | 102628910 | 102162573 | 14:102628910 | T | TTCT | 0.0453 | 0.0099 | 1.0099 | 1.0009 |
| | TC_T | | | _TTCTC_T | | C | | | | |
| 14 | 14_103238221_G_ | 103238221 | 102771884 | rs72702772 | A | G | 0.2035 | −0.0086 | 0.9914 | 0.9965 |
| | A | | | | | | | | | |
| 14 | 14_103381882_C_T | 103381882 | 102915545 | rs72704741 | T | C | 0.1737 | −0.0058 | 0.9942 | 0.9980 |
| 14 | 14_104374675_G_ | 104374675 | 103908338 | | T | G | 0.0171 | 0.0044 | 1.0044 | 1.0002 |
| | T | | | | | | | | | |
| 14 | 14_104837529_T_C | 104837529 | 104371192 | rs61577366 | C | T | 0.3982 | −0.0099 | 0.9901 | 0.9922 |
| 14 | 14 105154105_A_ | 105154105 | 104687768 | rs3809456 | G | A | 0.5121 | 0.0044 | 1.0044 | 1.0045 |
| | G | | | | | | | | | |
| 14 | 14_105213978_T_ | 105213978 | 104747641 | rs4983544 | G | T | 0.4588 | 0.0154 | 1.0155 | 1.0143 |
| | G | | | | | | | | | |
| 14 | 14_105320853_C_T | 105320853 | 104854516 | rs148957187 | T | C | 0.5973 | −0.0030 | 0.9970 | 0.9964 |
| 15 | 15_22944416_T_A | 22944416 | 22928652 | rs28439038 | A | T | 0.2763 | 0.0127 | 1.0128 | 1.0071 |
| 15 | 15_25498397_A_G | 25498397 | 25253250 | rs6576416 | G | A | 0.5524 | 0.0028 | 1.0028 | 1.0031 |
| 15 | 15_25978812_G_A | 25978812 | 25733665 | rs61998562 | A | G | 0.3916 | 0.0104 | 1.0105 | 1.0082 |
| 15 | 15_26015425_C_G | 26015425 | 25770278 | rs28627117 | G | C | 0.2125 | 0.0049 | 1.0049 | 1.0021 |
| 15 | 15_30211357_T_C | 30211357 | 29919154 | rs1719339 | C | T | 0.0265 | 0.0303 | 1.0308 | 1.0016 |
| 15 | 15_30906165_T_C | 30906165 | 30613962 | rs200113015 | C | T | 0.2334 | 0.0000 | 1.0000 | 1.0000 |
| 15 | 15_30913755_A_T | 30913755 | 30621552 | rs62018335 | T | A | 0.2680 | 0.0126 | 1.0127 | 1.0068 |
| 15 | 15_31802360_G_A | 31802360 | 31510157 | rs12441163 | A | G | 0.1404 | −0.0105 | 0.9896 | 0.9971 |
| 15 | 15_31808639_C_T | 31808639 | 31516436 | rs4779888 | T | C | 0.2795 | −0.0008 | 0.9992 | 0.9996 |
| 15 | 15_32191559_T_C | 32191559 | 31899356 | rs117164250 | C | T | 0.0565 | −0.0156 | 0.9845 | 0.9983 |
| 15 | 15_32941233_A_C | 32941233 | 32649032 | rs113137317 | C | A | 0.0238 | 0.0446 | 1.0456 | 1.0022 |
| 15 | 15_35753643_A_G | 35753643 | 35461442 | rs16961010 | G | A | 0.0857 | 0.0069 | 1.0069 | 1.0012 |
| 15 | 15_37466649_G_A | 37466649 | 37174448 | rs12911895 | A | G | 0.0769 | 0.0077 | 1.0077 | 1.0012 |
| 15 | 15_38125629_G_T | 38125629 | 37833428 | rs12907082 | T | G | 0.3992 | −0.0040 | 0.9960 | 0.9968 |
| 15 | 15_38156880_G_A | 38156880 | 37864679 | rs35103746 | A | G | 0.1290 | −0.0044 | 0.9956 | 0.9989 |
| 15 | 15_38329343_G_A | 38329343 | 38037142 | rs6495940 | A | G | 0.8894 | 0.0051 | 1.0051 | 1.0091 |
| 15 | 15_40130800_A_A | 40130800 | 39838599 | rs573759836 | AGAT | A | 0.0178 | −0.0149 | 0.9852 | 0.9995 |
| | GAT | | | | | | | | | |
| 15 | 15_40889198_CAA | 40889198 | 40597000 | rs774126232 | C | CAA | 0.7748 | 0.0114 | 1.0115 | 1.0178 |
| | _C | | | | | | | | | |
| 15 | 15_40905557_T_C | 40905557 | 40613359 | rs17747351 | C | T | 0.1316 | −0.0023 | 0.9977 | 0.9994 |
| 15 | 15_40992997_CA_ | 40992997 | 40700799 | 15:40992997_ | C | CA | 0.1325 | −0.0020 | 0.9980 | 0.9995 |
| | C | | | CA_C | | | | | | |
| 15 | 15_41097573_CAA | 41097573 | 40805375 | 15:41097573_ | C | CAAA | 0.3868 | 0.0011 | 1.0011 | 1.0009 |
| | AAAAAA_C | | | CAAAAAAAA_ | | AAAA | | | | |
| | | | | C | | A | | | | |
| 15 | 15_43534967_A_G | 43534967 | 43242769 | rs11855754 | G | A | 0.0990 | −0.0100 | 0.9900 | 0.9980 |
| 15 | 15_43535096_A_G | 43535096 | 43242898 | rs525403 | G | A | 0.7070 | 0.0018 | 1.0018 | 1.0025 |
| 15 | 15_44216559_C_CT | 44216559 | 43924361 | rs5812256 | CT | C | 0.3255 | −0.0109 | 0.9892 | 0.9930 |
| 15 | 15_44438763_T_C | 44438763 | 44146565 | rs2615272 | C | T | 0.9671 | 0.0171 | 1.0172 | 1.0336 |
| 15 | 15_44474906_T_C | 44474906 | 44182708 | rs2256928 | C | T | 0.1506 | −0.0003 | 0.9997 | 0.9999 |
| 15 | 15_44497361_A_A | 44497361 | 44205163 | rs796947891 | AG | A | 0.0638 | 0.0000 | 1.0000 | 1.0000 |
| | G | | | | | | | | | |
| 15 | 15_44514657_T_A | 44514657 | 44222459 | rs2464693 | A | T | 0.5896 | 0.0021 | 1.0021 | 1.0025 |
| 15 | 15_45488821_C_T | 45488821 | 45196623 | rs62026664 | T | C | 0.3464 | 0.0054 | 1.0054 | 1.0038 |
| 15 | 15_45513647_G_A | 45513647 | 45221449 | rs10851421 | A | G | 0.3071 | 0.0021 | 1.0021 | 1.0013 |
| 15 | 15_46680811_C_A | 46680811 | 46388613 | rs187010898 | A | C | 0.0115 | −0.0125 | 0.9876 | 0.9997 |
| 15 | 15_46788586_C_T | 46788586 | 46496388 | rs147041270 | T | C | 0.0131 | −0.0754 | 0.9274 | 0.9981 |
| 15 | 15_47059047_CT_C | 47059047 | 46766849 | rs773759620 | C | CT | 0.6689 | −0.0001 | 0.9999 | 0.9999 |
| 15 | 15_47675569_G_A | 47675569 | 47383372 | rs281264 | A | G | 0.6481 | −0.0028 | 0.9972 | 0.9964 |
| 15 | 15_47685504_T_C | 47685504 | 47393307 | rs281297 | C | T | 0.3794 | 0.0051 | 1.0051 | 1.0039 |
| 15 | 15_47686081_C_T | 47686081 | 47393884 | rs281299 | T | C | 0.6170 | 0.0024 | 1.0024 | 1.0030 |
| 15 | 15_47686590_C_T | 47686590 | 47394393 | rs11070582 | T | C | 0.1343 | 0.0047 | 1.0047 | 1.0013 |
| 15 | 15_47702127_T_C | 47702127 | 47409930 | rs2433020 | C | T | 0.5264 | 0.0051 | 1.0051 | 1.0054 |
| 15 | 15_47716150_A_G | 47716150 | 47423953 | rs11632616 | G | A | 0.3103 | 0.0065 | 1.0065 | 1.0041 |
| 15 | 15_50342912_A_G | 50342912 | 50050715 | rs138654651 | G | A | 0.0109 | 0.0000 | 1.0000 | 1.0000 |
| 15 | 15_50663903_G_A | 50663903 | 50371706 | rs34565064 | A | G | 0.3554 | −0.0046 | 0.9954 | 0.9967 |
| 15 | 15_50667009_G_A | 50667009 | 50374812 | rs12905736 | A | G | 0.2801 | −0.0021 | 0.9979 | 0.9988 |
| 15 | 15_50672808_C_G | 50672808 | 50380611 | rs4775879 | G | C | 0.5622 | −0.0016 | 0.9984 | 0.9982 |
| 15 | 15_50673281_T_C | 50673281 | 50381084 | rs34174311 | C | T | 0.3492 | −0.0047 | 0.9953 | 0.9967 |
| 15 | 15_50694608_C_A | 50694608 | 50402411 | rs11070772 | A | C | 0.3876 | 0.0048 | 1.0048 | 1.0037 |
| 15 | 15_51383131_G_G | 51383131 | 51090934 | rs60211045 | GA | G | 0.3980 | −0.0020 | 0.9980 | 0.9984 |
| | A | | | | | | | | | |
| 15 | 15_51493515_CT_C | 51493515 | 51201318 | 15:51493515_ | C | CT | 0.8054 | 0.0069 | 1.0069 | 1.0112 |
| | | | | CT_C | | | | | | |
| 15 | 15_51498587_G_T | 51498587 | 51206390 | rs6493485 | T | G | 0.8241 | 0.0060 | 1.0060 | 1.0099 |
| 15 | 15_51498708_A_G | 51498708 | 51206511 | rs6493486 | G | A | 0.7334 | 0.0020 | 1.0020 | 1.0029 |
| 15 | 15_51527398_A_G | 51527398 | 51235201 | rs3784308 | G | A | 0.0428 | 0.0085 | 1.0085 | 1.0007 |
| 15 | 15_52384414_C_T | 52384414 | 52092217 | rs113379345 | T | C | 0.0820 | −0.0160 | 0.9841 | 0.9974 |
| 15 | 15_54640812_G_A | 54640812 | 54348614 | rs62012035 | A | G | 0.0413 | 0.0168 | 1.0169 | 1.0014 |
| 15 | 15_55609126_G_A | 55609126 | 55316928 | rs116918550 | A | G | 0.0161 | −0.0161 | 0.9840 | 0.9995 |
| 15 | 15_55626817_C_T | 55626817 | 55334619 | rs4774764 | T | C | 0.2288 | 0.0083 | 1.0083 | 1.0038 |
| 15 | 15_56073123_A_G | 56073123 | 55780925 | rs149316549 | G | A | 0.1592 | −0.0004 | 0.9996 | 0.9999 |
| 15 | 15_56090609_C_T | 56090609 | 55798411 | rs17238405 | T | C | 0.3252 | −0.0071 | 0.9929 | 0.9954 |
| 15 | 15_56114197_G_A | 56114197 | 55821999 | rs34555597 | A | G | 0.2014 | 0.0050 | 1.0050 | 1.0020 |
| 15 | 15_56283412_C_T | 56283412 | 55991214 | rs17238531 | T | C | 0.0158 | 0.0196 | 1.0198 | 1.0006 |
| 15 | 15_57671246_T_G | 57671246 | 57379048 | rs1664471 | G | T | 0.6118 | 0.0004 | 1.0004 | 1.0005 |
| 15 | 15_57999304_G_C | 57999304 | 57707106 | rs11858659 | C | G | 0.0596 | −0.0199 | 0.9803 | 0.9977 |
| 15 | 15_59280279_G_A | 59280279 | 58988080 | rs12905422 | A | G | 0.2189 | −0.0012 | 0.9988 | 0.9995 |
| 15 | 15_59393174_T_G | 59393174 | 59100975 | rs59025291 | G | T | 0.0760 | −0.0228 | 0.9775 | 0.9966 |
| 15 | 15_60342686_T_G | 60342686 | 60050487 | rs2899654 | G | T | 0.0121 | −0.0448 | 0.9562 | 0.9989 |
| 15 | 15_62808539_C_T | 62808539 | 62516340 | rs956006 | T | C | 0.3342 | 0.0092 | 1.0092 | 1.0062 |
| 15 | 15_63351451_C_T | 63351451 | 63059252 | rs78809927 | T | C | 0.0136 | −0.0299 | 0.9705 | 0.9992 |
| 15 | 15_63742901_A_G | 63742901 | 63450702 | rs12907670 | G | A | 0.8421 | 0.0102 | 1.0103 | 1.0173 |
| 15 | 15_63758647_C_T | 63758647 | 63466448 | rs4984247 | T | C | 0.1359 | −0.0044 | 0.9956 | 0.9988 |
| 15 | 15_63810625_G_C | 63810625 | 63518426 | rs72750932 | C | G | 0.1445 | 0.0103 | 1.0104 | 1.0030 |
| 15 | 15_64369496_CAA | 64369496 | 64077297 | | CAACAAA | CAAC | 0.6938 | 0.0118 | 1.0119 | 1.0165 |
| | CAA_CAACAAAACA | | | | ACAA | AA | | | | |
| | A | | | | | | | | | |
| 15 | 15_65010075_G_A | 65010075 | 64717876 | rs16948203 | A | G | 0.0654 | −0.0027 | 0.9973 | 0.9996 |
| 15 | 15_66570312_GA_ | 66570312 | 66277974 | | G | GA | 0.3334 | 0.0024 | 1.0024 | 1.0016 |
| | G | | | | | | | | | |
| 15 | 15_66802208_T_G | 66802208 | 66509870 | rs12905354 | G | T | 0.3353 | 0.0084 | 1.0084 | 1.0057 |
| 15 | 15_66861719_G_A | 66861719 | 66569381 | rs8038632 | A | G | 0.6668 | −0.0041 | 0.9959 | 0.9946 |
| 15 | 15_66891819_G_A | 66891819 | 66599481 | rs144711219 | A | G | 0.0293 | −0.0193 | 0.9809 | 0.9989 |
| 15 | 15_67457698_A_G | 67457698 | 67165360 | rs35874463 | G | A | 0.0496 | 0.0173 | 1.0175 | 1.0017 |
| 15 | 15_67762759_G_A | 67762759 | 67470421 | rs150073640 | A | G | 0.0159 | 0.0303 | 1.0308 | 1.0010 |
| 15 | 15_69965730_G_A | 69965730 | 69673391 | rs59384564 | A | G | 0.2322 | 0.0000 | 1.0000 | 1.0000 |
| 15 | 15_74487024_A_G | 74487024 | 74194683 | rs58138979 | G | A | 0.1055 | 0.0179 | 1.0181 | 1.0038 |
| 15 | 15_75059627_A_G | 75059627 | 74767286 | rs12903541 | G | A | 0.6503 | 0.0044 | 1.0044 | 1.0057 |
| 15 | 15_75145098_T_C | 75145098 | 74852757 | rs936230 | C | T | 0.6997 | 0.0081 | 1.0081 | 1.0114 |
| 15 | 15_75750383_T_C | 75750383 | 75458042 | rs8035987 | C | T | 0.2604 | −0.0168 | 0.9833 | 0.9913 |
| 15 | 15_75932777_C_T | 75932777 | 75640436 | rs11072553 | T | C | 0.2480 | −0.0027 | 0.9973 | 0.9987 |
| 15 | 15_86140791_G_A | 86140791 | 85597560 | rs17633749 | A | G | 0.3429 | 0.0000 | 1.0000 | 1.0000 |
| 15 | 15_86298852_A_G | 86298852 | 85755621 | rs2542606 | G | A | 0.5896 | 0.0089 | 1.0089 | 1.0106 |
| 15 | 15_86300805_T_TT | 86300805 | 85757574 | rs547514224 | TTCC | T | 0.3141 | −0.0024 | 0.9976 | 0.9985 |
| | CC | | | | | | | | | |
| 15 | 15_86832749_G_A | 86832749 | 86289518 | rs12904524 | A | G | 0.2828 | −0.0061 | 0.9939 | 0.9966 |
| 15 | 15_86840700_C_T | 86840700 | 86297469 | rs8038617 | T | C | 0.3452 | −0.0018 | 0.9982 | 0.9988 |
| 15 | 15_88502947_C_T | 88502947 | 87959716 | rs76289332 | T | C | 0.0512 | 0.0069 | 1.0069 | 1.0007 |
| 15 | 15_90054067_T_C | 90054067 | 89510836 | rs2119009 | C | T | 0.0710 | 0.0175 | 1.0177 | 1.0025 |
| 15 | 15_90095006_G_A | 90095006 | 89551775 | rs138481434 | A | G | 0.0238 | 0.0116 | 1.0117 | 1.0006 |
| 15 | 15_90122062_T_C | 90122062 | 89578831 | rs118118631 | C | T | 0.0797 | 0.0044 | 1.0044 | 1.0007 |
| 15 | 15_90768959_A_C | 90768959 | 90225727 | rs908044 | C | A | 0.3416 | −0.0024 | 0.9976 | 0.9984 |
| 15 | 15_91426560_G_A | 91426560 | 90883330 | rs4702 | A | G | 0.5581 | −0.0092 | 0.9908 | 0.9898 |
| 15 | 15_91512267_G_T | 91512267 | 90969037 | rs2290202 | T | G | 0.1353 | −0.0220 | 0.9782 | 0.9941 |
| 15 | 15_94513089_C_G | 94513089 | 93969860 | rs74630476 | G | C | 0.0140 | 0.0331 | 1.0337 | 1.0009 |
| 15 | 15_94534591_G_A | 94534591 | 93991362 | rs2009580 | A | G | 0.5807 | −0.0112 | 0.9889 | 0.9871 |
| 15 | 15_94550152_A_G | 94550152 | 94006923 | rs11074245 | G | A | 0.4716 | −0.0012 | 0.9988 | 0.9989 |
| 15 | 15_95675091_T_G | 95675091 | 95131862 | rs28675767 | G | T | 0.1211 | −0.0074 | 0.9926 | 0.9982 |
| 15 | 15_97887273_G_T | 97887273 | 97344043 | rs62025230 | T | G | 0.1526 | 0.0000 | 1.0000 | 1.0000 |
| 15 | 15_97944913_G_A | 97944913 | 97401683 | rs7181086 | A | G | 0.6006 | 0.0099 | 1.0099 | 1.0120 |
| 15 | 15_99610212_G_A | 99610212 | 99066983 | rs34097694 | A | G | 0.1882 | 0.0151 | 1.0152 | 1.0057 |
| 15 | 15_100572999_G_ | 100572999 | 100032794 | rs79270656 | A | G | 0.0858 | 0.0062 | 1.0062 | 1.0011 |
| | A | | | | | | | | | |
| 15 | 15_100891226_C_T | 100891226 | 100351021 | rs74917211 | T | C | 0.0319 | −0.0113 | 0.9888 | 0.9993 |
| 15 | 15_100905819_A_C | 100905819 | 100365614 | rs144767203 | C | A | 0.1100 | −0.0145 | 0.9856 | 0.9968 |
| 15 | 15_101087943_G_ | 101087943 | 100547738 | rs4965671 | A | G | 0.8706 | 0.0018 | 1.0018 | 1.0031 |
| | A | | | | | | | | | |
| 15 | 15_101140715_G_ | 101140715 | 100600510 | rs76361074 | A | G | 0.0632 | −0.0166 | 0.9835 | 0.9979 |
| | A | | | | | | | | | |
| 15 | 15_101158823_G_ | 101158823 | 100618618 | rs12907972 | T | G | 0.3695 | −0.0010 | 0.9990 | 0.9993 |
| | T | | | | | | | | | |
| 16 | 16_1407441_A_G | 1407441 | 1357440 | rs6600137 | G | A | 0.8928 | −0.0017 | 0.9983 | 0.9970 |
| 16 | 16_3980445_T_C | 3980445 | 3930444 | rs8049367 | C | T | 0.3914 | 0.0036 | 1.0036 | 1.0028 |
| 16 | 16_4008542_CAAA | 4008542 | 3958541 | 16:4008542_C | C | CAAA | 0.8213 | 0.0000 | 1.0000 | 1.0000 |
| | AA_C | | | AAAAA_C | | AA | | | | |
| 16 | 16_4008901_C_G | 4008901 | 3958900 | rs7199977 | G | C | 0.7785 | 0.0000 | 1.0000 | 1.0000 |
| 16 | 16_4015316_A_G | 4015316 | 3965315 | rs1045475 | G | A | 0.8238 | 0.0094 | 1.0094 | 1.0156 |
| 16 | 16_4034640_G_C | 4034640 | 3984639 | rs61124411 | C | G | 0.1029 | −0.0065 | 0.9935 | 0.9987 |
| 16 | 16_4064637_G_C | 4064637 | 4014636 | rs62036957 | C | G | 0.9198 | 0.0057 | 1.0057 | 1.0105 |
| 16 | 16_4071508_G_A | 4071508 | 4021507 | rs4785949 | A | G | 0.0168 | −0.0188 | 0.9814 | 0.9994 |
| 16 | 16_4106788_C_A | 4106788 | 4056787 | rs11076805 | A | C | 0.2643 | 0.0000 | 1.0000 | 1.0000 |
| 16 | 16_4108158_TA_T | 4108158 | 4058157 | 16:4108158_T | T | TA | 0.5053 | 0.0070 | 1.0070 | 1.0071 |
| | | | | A_T | | | | | | |
| 16 | 16_4133076_C_A | 4133076 | 4083075 | rs7193552 | A | C | 0.4968 | 0.0046 | 1.0046 | 1.0046 |
| 16 | 16_4161270_TG_T | 4161270 | 4111269 | 16:4161270_T | T | TG | 0.1104 | 0.0000 | 1.0000 | 1.0000 |
| | | | | G_T | | | | | | |
| 16 | 16_4171880_A_G | 4171880 | 4121879 | rs6500580 | G | A | 0.5202 | 0.0018 | 1.0018 | 1.0019 |
| 16 | 16_4182495_C_G | 4182495 | 4132494 | rs231611 | G | C | 0.4471 | 0.0000 | 1.0000 | 1.0000 |
| 16 | 16_4250892_A_C | 4250892 | 4200891 | rs879150 | C | A | 0.8543 | 0.0060 | 1.0060 | 1.0103 |
| 16 | 16_4958716_A_AT | 4958716 | 4908715 | rs143780013 | AT | A | 0.2036 | −0.0022 | 0.9978 | 0.9991 |
| 16 | 16_5432429_T_C | 5432429 | 5382428 | rs568903 | C | T | 0.5663 | −0.0037 | 0.9963 | 0.9958 |
| 16 | 16_5460523_T_C | 5460523 | 5410522 | rs561856 | C | T | 0.6247 | −0.0058 | 0.9942 | 0.9928 |
| 16 | 16_5710377_A_G | 5710377 | 5660376 | rs12598977 | G | A | 0.1120 | 0.0044 | 0.9956 | 0.9990 |
| 16 | 16_5725267_T_A | 5725267 | 5675266 | rs2079740 | A | T | 0.1220 | −0.0015 | 0.9985 | 0.9996 |
| 16 | 16_6963972_C_G | 6963972 | 6913971 | rs12709163 | G | C | 0.7835 | 0.0136 | 1.0137 | 1.0216 |
| 16 | 16_6971642_ATAT | 6971642 | 6921641 | rs869118530 | A | ATAT | 0.7591 | 0.0026 | 1.0026 | 1.0040 |
| | AT_A | | | | | AT | | | | |
| 16 | 16_6980296_T_C | 6980296 | 6930295 | rs4786951 | C | T | 0.5977 | 0.0003 | 1.0003 | 1.0004 |
| 16 | 16_6980653_C_T | 6980653 | 6930652 | rs117771295 | T | C | 0.1106 | 0.0066 | 1.0066 | 1.0015 |
| 16 | 16_7590853_C_G | 7590853 | 7540851 | rs12934004 | G | C | 0.7156 | −0.0026 | 0.9974 | 0.9963 |
| 16 | 16_7596179_A_G | 7596179 | 7546177 | rs12928189 | G | A | 0.7460 | −0.0029 | 0.9971 | 0.9957 |
| 16 | 16_7893537_T_C | 7893537 | 7843535 | rs7198296 | C | T | 0.1396 | 0.0136 | 1.0137 | 1.0038 |
| 16 | 16_8772835_G_C | 8772835 | 8678978 | rs150465260 | C | G | 0.0138 | 0.0282 | 1.0286 | 1.0008 |
| 16 | 16_10706580_G_A | 10706580 | 10612723 | rs34872983 | A | G | 0.0695 | −0.0262 | 0.9741 | 0.9964 |
| 16 | 16_11190930_T_C | 11190930 | 11097073 | rs11649025 | C | T | 0.1088 | −0.0261 | 0.9742 | 0.9944 |
| 16 | 16_11439679_C_G | 11439679 | 11345822 | rs918738 | G | C | 0.5100 | 0.0014 | 1.0014 | 1.0014 |
| 16 | 16_13625515_G_A | 13625515 | 13531658 | rs11864841 | A | G | 0.0688 | 0.0125 | 1.0126 | 1.0017 |
| 16 | 16_19334821_C_T | 19334821 | 19323499 | rs8059096 | T | C | 0.1012 | −0.0143 | 0.9858 | 0.9971 |
| 16 | 16_19505813_T_A | 19505813 | 19494491 | rs2353696 | A | T | 0.7385 | 0.0000 | 1.0000 | 1.0000 |
| 16 | 16_23007047_G_T | 23007047 | 22995726 | rs75753503 | T | G | 0.0236 | 0.0329 | 1.0334 | 1.0016 |
| 16 | 16_23009777_A_C | 23009777 | 22998456 | rs10459775 | C | A | 0.6944 | −0.0063 | 0.9937 | 0.9913 |
| 16 | 16_24116697_A_T | 24116697 | 24105376 | rs9746011 | T | A | 0.1490 | −0.0008 | 0.9992 | 0.9998 |
| 16 | 16_24117477_C_T | 24117477 | 24106156 | rs2470688 | T | C | 0.5360 | 0.0033 | 1.0033 | 1.0035 |
| 16 | 16_24124184_G_A | 24124184 | 24112863 | rs11646540 | A | G | 0.0502 | −0.0220 | 0.9782 | 0.9978 |
| 16 | 16_25347678_G_A | 25347678 | 25336357 | rs2112816 | A | G | 0.8529 | 0.0059 | 1.0059 | 1.0101 |
| 16 | 16_26730593_A_G | 26730593 | 26719272 | rs80216903 | G | A | 0.0361 | 0.0094 | 1.0094 | 1.0007 |
| 16 | 16_26737749_TC_T | 26737749 | 26726428 | 16:26737749_ | T | TC | 0.2995 | −0.0025 | 0.9975 | 0.9985 |
| | | | | TC_T | | | | | | |
| 16 | 16_26740721_G_A | 26740721 | 26729400 | rs321460 | A | G | 0.7547 | −0.0069 | 0.9931 | 0.9896 |
| 16 | 16_48923223_C_G | 48923223 | 48889312 | rs10521172 | G | C | 0.0511 | 0.0054 | 1.0054 | 1.0006 |
| 16 | 16_48954205_T_C | 48954205 | 48920294 | rs16946656 | C | T | 0.0388 | 0.0000 | 1.0000 | 1.0000 |
| 16 | 16_49929346_A_G | 49929346 | 49895435 | rs62029447 | G | A | 0.0946 | −0.0041 | 0.9959 | 0.9992 |
| 16 | 16_50493430_A_G | 50493430 | 50459519 | rs4785426 | G | A | 0.7513 | 0.0074 | 1.0074 | 1.0112 |
| 16 | 16_50612108_A_G | 50612108 | 50578197 | rs80279569 | G | A | 0.0203 | 0.0257 | 1.0260 | 1.0011 |
| 16 | 16_50912801_G_T | 50912801 | 50878890 | rs16949000 | T | G | 0.1983 | 0.0055 | 1.0055 | 1.0022 |
| 16 | 16_51689434_G_A | 51689434 | 51655523 | rs73593554 | A | G | 0.1051 | −0.0015 | 0.9985 | 0.9997 |
| 16 | 16_51692074_C_T | 51692074 | 51658163 | rs8053944 | T | C | 0.2283 | −0.0005 | 0.9995 | 0.9998 |
| 16 | 16_51737524_A_G | 51737524 | 51703613 | rs112648548 | G | A | 0.1441 | −0.0088 | 0.9912 | 0.9975 |
| 16 | 16_51752251_T_A | 51752251 | 51718340 | rs1381452 | A | T | 0.7274 | 0.0095 | 1.0095 | 1.0139 |
| 16 | 16_51754407_C_T | 51754407 | 51720496 | rs7190518 | T | C | 0.0774 | −0.0057 | 0.9943 | 0.9991 |
| 16 | 16_51976568_A_G | 51976568 | 51942656 | rs12932954 | G | A | 0.2484 | −0.0012 | 0.9988 | 0.9994 |
| 16 | 16_52415071_G_A | 52415071 | 52381159 | rs140770615 | A | G | 0.0538 | −0.0017 | 0.9983 | 0.9998 |
| 16 | 16_52535810_T_C | 52535810 | 52501898 | rs4784220 | C | T | 0.3666 | 0.0000 | 1.0000 | 1.0000 |
| 16 | 16_52538825_C_A | 52538825 | 52504913 | rs35668161 | A | C | 0.2562 | 0.1017 | 1.1071 | 1.0556 |
| 16 | 16_52573732_C_T | 52573732 | 52539820 | rs34928352 | T | C | 0.0245 | −0.0102 | 0.9899 | 0.9995 |
| 16 | 16_52586477_A_G | 52586477 | 52552565 | rs3803661 | G | A | 0.7310 | 0.0000 | 1.0000 | 1.0000 |
| 16 | 16_52599188_C_T | 52599188 | 52565276 | rs4784227 | T | C | 0.2406 | 0.0860 | 1.0898 | 1.0437 |
| 16 | 16_53167951_A_G | 53167951 | 53134039 | rs117216872 | G | A | 0.0337 | −0.0085 | 0.9915 | 0.9994 |
| 16 | 16_53795665_CAA | 53795665 | 53761753 | rs759431941 | C | CAAC | 0.1766 | 0.0043 | 1.0043 | 1.0015 |
| | CT_C | | | | | T | | | | |
| 16 | 16_53797908_C_G | 53797908 | 53763996 | rs7206790 | G | C | 0.4793 | −0.0001 | 0.9999 | 0.9999 |
| 16 | 16_53798622_G_T | 53798622 | 53764710 | rs8047587 | T | G | 0.4539 | −0.0127 | 0.9874 | 0.9886 |
| 16 | 16_53809123_C_T | 53809123 | 53775211 | rs55872725 | T | C | 0.4201 | −0.0353 | 0.9653 | 0.9711 |
| 16 | 16_53850170_A_G | 53850170 | 53816258 | rs1421090 | G | A | 0.2604 | 0.0000 | 1.0000 | 1.0000 |
| 16 | 16_53861139_C_T | 53861139 | 53827227 | rs6499648 | T | C | 0.7604 | 0.0000 | 1.0000 | 1.0000 |
| 16 | 16_53861592_G_A | 53861592 | 53827680 | rs7184573 | A | G | 0.3663 | −0.0186 | 0.9816 | 0.9865 |
| 16 | 16_53865368_A_A | 53865368 | 53831456 | rs34853502 | AG | A | 0.7189 | −0.0145 | 0.9856 | 0.9794 |
| | G | | | | | | | | | |
| 16 | 16_53871626_CTG | 53871626 | 53837714 | 16:53871626_ | C | CTG | 0.1564 | −0.0020 | 0.9980 | 0.9994 |
| | _C | | | CTG_C | | | | | | |
| 16 | 16_54114262_T_C | 54114262 | 54080350 | rs11646290 | C | T | 0.3275 | 0.0109 | 1.0110 | 1.0072 |
| 16 | 16_54679668_G_C | 54679668 | 54645756 | rs7205346 | C | G | 0.5980 | 0.0047 | 1.0047 | 1.0056 |
| 16 | 16_54679720_CTTT | 54679720 | 54645808 | 16:54679720_ | C | CTTT | 0.1587 | 0.0012 | 1.0012 | 1.0004 |
| | _C | | | CTTT_C | | | | | | |
| 16 | 16_54682064_G_A | 54682064 | 54648152 | rs28539243 | A | G | 0.4850 | 0.0218 | 1.0220 | 1.0215 |
| 16 | 16_54683802_T_C | 54683802 | 54649890 | rs3893264 | C | T | 0.1948 | 0.0009 | 1.0009 | 1.0004 |
| 16 | 16_56379792_C_T | 56379792 | 56345880 | rs3790110 | T | C | 0.5968 | −0.0073 | 0.9927 | 0.9913 |
| 16 | 16_56379937_C_T | 56379937 | 56346025 | rs13337678 | T | C | 0.5599 | 0.0000 | 1.0000 | 1.0000 |
| 16 | 16_56437872_T_A | 56437872 | 56403960 | rs2432542 | A | T | 0.5165 | −0.0008 | 0.9992 | 0.9992 |
| 16 | 16_57673762_G_A | 57673762 | 57639850 | rs1975630 | A | G | 0.5416 | −0.0043 | 0.9957 | 0.9954 |
| 16 | 16_61911209_T_TA | 61911209 | 61877305 | rs576519137 | TA | T | 0.2284 | 0.0000 | 1.0000 | 1.0000 |
| 16 | 16_61919615_CA_ | 61919615 | 61885711 | 16:61919615_ | C | CA | 0.4080 | 0.0098 | 1.0098 | 1.0081 |
| | C | | | CA_C | | | | | | |
| 16 | 16_63026665_G_A | 63026665 | 62992761 | rs567866625 | A | G | 0.0128 | 0.0391 | 1.0399 | 1.0010 |
| 16 | 16_63250011_T_C | 63250011 | 63216107 | rs12597559 | C | T | 0.4320 | 0.0133 | 1.0134 | 1.0116 |
| 16 | 16_63360933_A_G | 63360933 | 63327029 | rs117910740 | G | A | 0.0120 | 0.0175 | 1.0177 | 1.0004 |
| 16 | 16_63401800_T_C | 63401800 | 63367896 | rs143470 | C | T | 0.4395 | 0.0018 | 1.0018 | 1.0016 |
| 16 | 16_63446812_C_T | 63446812 | 63412908 | rs7189722 | T | C | 0.3580 | 0.0021 | 1.0021 | 1.0015 |
| 16 | 16_65310299_A_G | 65310299 | 65276396 | rs147958546 | G | A | 0.0512 | 0.0027 | 1.0027 | 1.0003 |
| 16 | 16_65430352_C_T | 65430352 | 65396449 | rs76021007 | T | C | 0.0759 | 0.0067 | 1.0067 | 1.0010 |
| 16 | 16_67003618_C_T | 67003618 | 66969715 | rs61743167 | T | C | 0.0219 | −0.0091 | 0.9909 | 0.9996 |
| 16 | 16_67181999_A_G | 67181999 | 67148096 | rs9033 | G | A | 0.4512 | 0.0101 | 1.0102 | 1.0092 |
| 16 | 16_71388416_G_A | 71388416 | 71354513 | rs142248381 | A | G | 0.0161 | −0.0183 | 0.9819 | 0.9994 |
| 16 | 16_71438287_A_C | 71438287 | 71404384 | rs8051854 | C | A | 0.3630 | 0.0096 | 1.0096 | 1.0070 |
| 16 | 16_71513034_C_A | 71513034 | 71479131 | rs2288486 | A | C | 0.5383 | −0.0073 | 0.9927 | 0.9922 |
| 16 | 16_71830922_T_C | 71830922 | 71797019 | rs61451008 | C | T | 0.4986 | −0.0092 | 0.9908 | 0.9909 |
| 16 | 16_72047385_A_G | 72047385 | 72013486 | rs138910759 | G | A | 0.0184 | −0.0182 | 0.9820 | 0.9993 |
| 16 | 16_73048109_G_C | 73048109 | 73014210 | rs12325298 | C | G | 0.4705 | −0.0105 | 0.9896 | 0.9902 |
| 16 | 16_75526936_C_A | 75526936 | 75493038 | rs2550901 | A | C | 0.0206 | 0.0337 | 1.0343 | 1.0014 |
| 16 | 16_78864207_T_TT | 78864207 | 78830310 | rs3085447 | TTA | T | 0.3175 | 0.0005 | 1.0005 | 1.0003 |
| | A | | | | | | | | | |
| 16 | 16_79223834_G_A | 79223834 | 79189937 | rs7186097 | A | G | 0.3076 | −0.0055 | 0.9945 | 0.9966 |
| 16 | 16_79238563_A_C | 79238563 | 79204666 | rs71398138 | C | A | 0.1492 | −0.0012 | 0.9988 | 0.9996 |
| 16 | 16_79330312_C_T | 79330312 | 79296415 | rs77471625 | T | C | 0.0547 | −0.0239 | 0.9764 | 0.9974 |
| 16 | 16_79362587_A_T | 79362587 | 79328690 | rs17724699 | T | A | 0.0455 | −0.0044 | 0.9956 | 0.9996 |
| 16 | 16_79816295_ATTT | 79816295 | 79782398 | rs143581936 | A | ATTT | 0.6870 | 0.0037 | 1.0037 | 1.0051 |
| | TTTT_A | | | | | TTTT | | | | |
| 16 | 16_80648296_A_G | 80648296 | 80614399 | rs7500067 | G | A | 0.2303 | 0.0542 | 1.0557 | 1.0258 |
| 16 | 16_80652246_G_C | 80652246 | 80618349 | rs11645914 | C | G | 0.1885 | 0.0001 | 1.0001 | 1.0000 |
| 16 | 16_80656966_G_T | 80656966 | 80623069 | rs9927571 | T | G | 0.2444 | 0.0027 | 1.0027 | 1.0013 |
| 16 | 16_80849567_T_C | 80849567 | 80815670 | rs12448585 | C | T | 0.1875 | −0.0035 | 0.9965 | 0.9987 |
| 16 | 16_81809272_T_C | 81809272 | 81775667 | rs78266002 | C | T | 0.0279 | 0.0270 | 1.0274 | 1.0015 |
| 16 | 16_82079091_G_A | 82079091 | 82045486 | rs6564961 | A | G | 0.6080 | −0.0105 | 0.9896 | 0.9873 |
| 16 | 16_82111279_C_A | 82111279 | 82077674 | rs67777781 | A | C | 0.3644 | 0.0016 | 1.0016 | 1.0012 |
| 16 | 16_82712315_A_G | 82712315 | 82678710 | rs10514555 | G | A | 0.1043 | 0.0036 | 1.0036 | 1.0008 |
| 16 | 16_82862855_C_T | 82862855 | 82829250 | rs2549157 | T | C | 0.3159 | 0.0000 | 1.0000 | 1.0000 |
| 16 | 16_82927367_A_G | 82927367 | 82893762 | rs1862681 | G | A | 0.3684 | 0.0000 | 1.0000 | 1.0000 |
| 16 | 16_84285777_T_C | 84285777 | 84252171 | rs390112 | C | T | 0.6256 | 0.0032 | 1.0032 | 1.0040 |
| 16 | 16_85144693_C_A | 85144693 | 85111087 | rs8060060 | A | C | 0.4520 | 0.0000 | 1.0000 | 1.0000 |
| 16 | 16_85145977_T_C | 85145977 | 85112371 | rs9931038 | C | T | 0.4856 | 0.0000 | 1.0000 | 1.0000 |
| 16 | 16_85828183_T_C | 85828183 | 85794577 | rs56306388 | C | T | 0.1097 | 0.0000 | 1.0000 | 1.0000 |
| 16 | 16_85876104_A_G | 85876104 | 85842498 | rs9674291 | G | A | 0.1449 | 0.0139 | 1.0140 | 1.0041 |
| 16 | 16_87044630_T_C | 87044630 | 87011024 | rs2313582 | C | T | 0.3038 | −0.0041 | 0.9959 | 0.9975 |
| 16 | 16_87046969_T_C | 87046969 | 87013363 | rs71390870 | C | T | 0.1719 | 0.0000 | 1.0000 | 1.0000 |
| 16 | 16_87053923_A_C | 87053923 | 87020317 | rs929859 | C | A | 0.1869 | −0.0076 | 0.9924 | 0.9972 |
| 16 | 16_87058866_T_C | 87058866 | 87025260 | rs12445782 | C | T | 0.2068 | 0.0000 | 1.0000 | 1.0000 |
| 16 | 16_87069983_G_T | 87069983 | 87036377 | rs3901356 | T | G | 0.2335 | 0.0000 | 1.0000 | 1.0000 |
| 16 | 16_87086492_T_C | 87086492 | 87052886 | rs12449271 | C | T | 0.2586 | −0.0073 | 0.9927 | 0.9962 |
| 16 | 16_87135504_G_A | 87135504 | 87101898 | rs1559336 | A | G | 0.2406 | −0.0078 | 0.9922 | 0.9963 |
| 16 | 16_88619156_G_A | 88619156 | 88552748 | rs72807472 | A | G | 0.0133 | 0.0227 | 1.0230 | 1.0006 |
| 16 | 16_88822095_C_G | 88822095 | 88755687 | rs4782346 | G | C | 0.3302 | −0.0109 | 0.9892 | 0.9929 |
| 16 | 16_89802396_G_T | 89802396 | 89735988 | rs7202427 | T | G | 0.2681 | 0.0028 | 1.0028 | 1.0015 |
| 16 | 16_89850170_C_T | 89850170 | 89783762 | rs17226274 | T | C | 0.0877 | 0.0003 | 1.0003 | 1.0001 |
| 16 | 16_89850776_CA_ | 89850776 | 89784368 | 16:89850776_ | C | CA | 0.3322 | −0.0056 | 0.9944 | 0.9963 |
| | C | | | CA_C | | | | | | |
| 16 | 16_89981345_TTG_ | 89981345 | 89914937 | | T | TTG | 0.0916 | 0.0170 | 1.0171 | 1.0031 |
| | T | | | | | | | | | |
| 16 | 16_89986608_A_G | 89986608 | 89920200 | rs2228478 | G | A | 0.1048 | 0.0019 | 1.0019 | 1.0004 |
| 17 | 17_1902751_AAC_ | 1902751 | 1999457 | 17:1902751_A | A | AAC | 0.6720 | −0.0123 | 0.9878 | 0.9836 |
| | A | | | AC_A | | | | | | |
| 17 | 17_2099183_C_T | 2099183 | 2195889 | rs16952006 | T | C | 0.1079 | −0.0137 | 0.9864 | 0.9971 |
| 17 | 17_2217876_CAA_ | 2217876 | 2314582 | 17:2217876_C | C | CAA | 0.5766 | −0.0016 | 0.9984 | 0.9982 |
| | C | | | AA_C | | | | | | |
| 17 | 17_3419690_A_G | 3419690 | 3516396 | rs8082339 | G | A | 0.9854 | −0.0294 | 0.9710 | 0.9437 |
| 17 | 17_4696873_C_A | 4696873 | 4793578 | rs12937934 | A | C | 0.6872 | −0.0127 | 0.9874 | 0.9827 |
| 17 | 17_4963726_CT_C | 4963726 | 5060431 | 17:4963726_C | C | CT | 0.6003 | −0.0034 | 0.9966 | 0.9959 |
| | | | | T_C | | | | | | |
| 17 | 17_6332522_C_T | 6332522 | 6429202 | rs140991377 | T | C | 0.0204 | 0.0072 | 1.0072 | 1.0003 |
| 17 | 17_6498498_C_T | 6498498 | 6595178 | rs4796339 | T | C | 0.5713 | −0.0088 | 0.9912 | 0.9900 |
| 17 | 17_7166932_T_TG | 7166932 | 7263613 | rs201780804 | TG | T | 0.6330 | 0.0111 | 1.0112 | 1.0142 |
| 17 | 17_7206781_G_A | 7206781 | 7303462 | rs62059194 | A | G | 0.4402 | 0.0024 | 1.0024 | 1.0021 |
| 17 | 17_7358000_C_G | 7358000 | 7454681 | rs4796418 | G | C | 0.1481 | 0.0069 | 1.0069 | 1.0021 |
| 17 | 17_8907415_A_C | 8907415 | 9004098 | rs11868829 | C | A | 0.3252 | 0.0039 | 1.0039 | 1.0025 |
| 17 | 17_8944220_C_T | 8944220 | 9040903 | rs117766280 | T | C | 0.0143 | 0.0537 | 1.0552 | 1.0016 |
| 17 | 17_11717399_G_T | 11717399 | 11814082 | rs11078033 | T | G | 0.4080 | 0.0087 | 1.0087 | 1.0071 |
| 17 | 17_17766877_CT_C | 17766877 | 17863563 | rs537436727 | C | CT | 0.6128 | −0.0036 | 0.9964 | 0.9956 |
| 17 | 17_17878524_CTT_ | 17878524 | 17975210 | 17:17878524_ | C | CTT | 0.3275 | 0.0096 | 1.0096 | 1.0063 |
| | C | | | CTT_C | | | | | | |
| 17 | 17_18227272_T_C | 18227272 | 18323958 | rs921985 | C | T | 0.6274 | 0.0062 | 1.0062 | 1.0078 |
| 17 | 17_25767765_T_C | 25767765 | 27440739 | rs117920301 | C | T | 0.5192 | −0.0021 | 0.9979 | 0.9978 |
| 17 | 17_26351978_G_A | 26351978 | 28024952 | rs77038763 | A | G | 0.0248 | 0.0337 | 1.0343 | 1.0017 |
| 17 | 17_27771132_G_G | 27771132 | 29444114 | rs569046653 | GA | G | 0.3291 | 0.0096 | 1.0096 | 1.0064 |
| | A | | | | | | | | | |
| 17 | 17_28653780_C_T | 28653780 | 30326762 | rs60450790 | T | C | 0.0627 | −0.0026 | 0.9974 | 0.9997 |
| 17 | 17_28867862_G_C | 28867862 | 30540844 | rs9911207 | C | G | 0.0704 | −0.0148 | 0.9853 | 0.9979 |
| 17 | 17_29163591_T_A | 29163591 | 30836573 | rs117662433 | A | T | 0.0387 | −0.0159 | 0.9842 | 0.9988 |
| 17 | 17_29168077_G_T | 29168077 | 30841059 | rs79461387 | T | G | 0.2613 | −0.0242 | 0.9761 | 0.9875 |
| 17 | 17_29271319_A_T | 29271319 | 30944301 | rs6505220 | T | A | 0.4505 | 0.0100 | 1.0101 | 1.0091 |
| 17 | 17_29325023_G_C | 29325023 | 30998005 | rs12453269 | C | G | 0.2326 | 0.0000 | 1.0000 | 1.0000 |
| 17 | 17_29848534_C_T | 29848534 | 31521516 | rs11653842 | T | C | 0.0554 | −0.0016 | 0.9984 | 0.9998 |
| 17 | 17_32542662_G_A | 32542662 | 34215643 | rs28843509 | A | G | 0.2229 | −0.0066 | 0.9934 | 0.9971 |
| 17 | 17_32622343_G_A | 32622343 | 34295324 | rs201838321 | A | G | 0.1166 | 0.0161 | 1.0162 | 1.0038 |
| 17 | 17_37949924_A_G | 37949924 | 39793671 | rs17676191 | G | A | 0.0979 | −0.0079 | 0.9921 | 0.9985 |
| 17 | 17_38105334_C_T | 38105334 | 39949081 | rs7217240 | T | C | 0.2278 | −0.0002 | 0.9998 | 0.9999 |
| 17 | 17_38124892_C_A | 38124892 | 39968639 | rs59843584 | A | C | 0.2307 | −0.0020 | 0.9980 | 0.9991 |
| 17 | 17_38149033_G_A | 38149033 | 39992780 | rs11652139 | A | G | 0.6017 | −0.0026 | 0.9974 | 0.9969 |
| 17 | 17_38211557_GT_ | 38211557 | 40055304 | rs776336978 | G | GT | 0.0796 | −0.0006 | 0.9994 | 0.9999 |
| | G | | | | | | | | | |
| 17 | 17_38238725_C_T | 38238725 | 40082472 | rs113855596 | T | C | 0.3348 | 0.0011 | 1.0011 | 1.0007 |
| 17 | 17_38251385_A_A | 38251385 | 40095132 | rs141144358 | AT | A | 0.3417 | 0.0094 | 1.0094 | 1.0065 |
| | T | | | | | | | | | |
| 17 | 17_39178958_T_A | 39178958 | 41022706 | rs146491350 | A | T | 0.0705 | 0.0140 | 1.0141 | 1.0020 |
| 17 | 17_39215439_G_A | 39215439 | 41059187 | rs113045371 | A | G | 0.1329 | 0.0063 | 1.0063 | 1.0017 |
| 17 | 17_39251123_T_C | 39251123 | 41094871 | rs150537328 | C | T | 0.0682 | 0.0151 | 1.0152 | 1.0021 |
| 17 | 17_39272523_A_G | 39272523 | 41116271 | rs111447797 | G | A | 0.2635 | 0.0044 | 1.0044 | 1.0023 |
| 17 | 17_39305956_G_A | 39305956 | 41149704 | rs1497383 | A | G | 0.4865 | 0.0011 | 1.0011 | 1.0011 |
| 17 | 17_39449318_T_C | 39449318 | 41293066 | rs191981806 | C | T | 0.0291 | 0.0000 | 1.0000 | 1.0000 |
| 17 | 17_40114587_G_A | 40114587 | 41962569 | rs9899099 | A | G | 0.1020 | 0.0000 | 1.0000 | 1.0000 |
| 17 | 17_40127060_T_C | 40127060 | 41975042 | rs11296 | C | T | 0.0570 | 0.0000 | 1.0000 | 1.0000 |
| 17 | 17_40485239_G_T | 40485239 | 42333221 | rs17881320 | T | G | 0.0874 | −0.0067 | 0.9933 | 0.9988 |
| 17 | 17_40534672_C_CT | 40534672 | 42382654 | rs541465138 | CT | C | 0.0948 | −0.0027 | 0.9973 | 0.9995 |
| 17 | 17_40569439_T_C | 40569439 | 42417421 | rs12938957 | C | T | 0.2155 | 0.0053 | 1.0053 | 1.0023 |
| 17 | 17_40574052_C_A | 40574052 | 42422034 | rs9909335 | A | C | 0.0862 | 0.0000 | 1.0000 | 1.0000 |
| 17 | 17_40599241_CA_ | 40599241 | 42447223 | 17:40599241_ | C | CA | 0.7133 | −0.0071 | 0.9929 | 0.9899 |
| | C | | | CA_C | | | | | | |
| 17 | 17_40744470_G_A | 40744470 | 42592452 | rs149370081 | A | G | 0.0124 | 0.0559 | 1.0575 | 1.0014 |
| 17 | 17_40756882_C_T | 40756882 | 42604864 | rs76189032 | T | C | 0.0722 | 0.0040 | 1.0040 | 1.0006 |
| 17 | 17_40867365_C_G | 40867365 | 42715347 | rs115818584 | G | C | 0.0149 | 0.0173 | 1.0175 | 1.0005 |
| 17 | 17_40893920_G_C | 40893920 | 42741902 | rs113025290 | C | G | 0.0652 | 0.0049 | 1.0049 | 1.0006 |
| 17 | 17_42362104_A_G | 42362104 | 44284736 | rs7213794 | G | A | 0.5371 | −0.0031 | 0.9969 | 0.9967 |
| 17 | 17_43212339_C_CT | 43212339 | 45134972 | rs545502941 | CT | C | 0.2284 | 0.0122 | 1.0123 | 1.0056 |
| 17 | 17_43811260_G_A | 43811260 | 45733894 | rs1880753 | A | G | 0.5450 | 0.0000 | 1.0000 | 1.0000 |
| 17 | 17_43929696_T_C | 43929696 | 45852330 | rs9890016 | C | T | 0.0773 | 0.0067 | 1.0067 | 1.0010 |
| 17 | 17_44067847_G_T | 44067847 | 45990481 | rs2435213 | T | G | 0.1953 | 0.0075 | 1.0075 | 1.0029 |
| 17 | 17_44252468_G_A | 44252468 | 46175102 | rs2532263 | A | G | 0.1877 | −0.0075 | 0.9925 | 0.9972 |
| 17 | 17_44283858_G_A | 44283858 | 46206492 | rs2668667 | A | G | 0.1895 | −0.0162 | 0.9839 | 0.9939 |
| 17 | 17_44877342_C_T | 44877342 | 46799976 | rs116332759 | T | C | 0.0143 | −0.0072 | 0.9928 | 0.9998 |
| 17 | 17_45385526_A_A | 45385526 | 47308160 | rs368542936 | AAATAAT | A | 0.4583 | 0.0047 | 1.0047 | 1.0043 |
| | AATAATAATAATAA | | | | AATAATA | | | | | |
| | TAAT | | | | ATAAT | | | | | |
| 17 | 17_45912302_C_T | 45912302 | 47834936 | rs11079797 | T | C | 0.1592 | 0.0008 | 1.0008 | 1.0003 |
| 17 | 17_46051911_G_A | 46051911 | 47974545 | rs886444 | A | G | 0.4015 | 0.0015 | 1.0015 | 1.0012 |
| 17 | 17_47286962_C_T | 47286962 | 49209600 | rs2233359 | T | C | 0.1005 | 0.0110 | 1.0111 | 1.0022 |
| 17 | 17_47316758_C_T | 47316758 | 49239396 | rs112075909 | T | C | 0.1032 | 0.0072 | 1.0072 | 1.0015 |
| 17 | 17_47793042_G_A | 47793042 | 49715680 | rs2017835 | A | G | 0.5735 | 0.0054 | 1.0054 | 1.0062 |
| 17 | 17_48244531_C_G | 48244531 | 50167170 | rs12939159 | G | C | 0.1274 | −0.0031 | 0.9969 | 0.9992 |
| 17 | 17_48251875_G_G | 48251875 | 50174514 | rs200753576 | GT | G | 0.2058 | 0.0025 | 1.0025 | 1.0010 |
| | T | | | | | | | | | |
| 17 | 17_49430739_T_C | 49430739 | 51353378 | rs138253853 | C | T | 0.0179 | 0.0073 | 1.0073 | 1.0003 |
| 17 | 17_49609158_C_T | 49609158 | 51531797 | rs11867780 | T | C | 0.3025 | −0.0077 | 0.9923 | 0.9954 |
| 17 | 17_49828922_C_T | 49828922 | 51751562 | rs1263945 | T | C | 0.4834 | −0.0082 | 0.9918 | 0.9921 |
| 17 | 17_52947714_T_G | 52947714 | 54870353 | rs4436838 | G | T | 0.2662 | −0.0086 | 0.9914 | 0.9954 |
| 17 | 17_52975265_C_G | 52975265 | 54897904 | rs72840963 | G | C | 0.1924 | −0.0007 | 0.9993 | 0.9997 |
| 17 | 17_53116708_T_TA | 53116708 | 55039347 | rs11463240 | TA | T | 0.7073 | 0.0000 | 1.0000 | 1.0000 |
| 17 | 17_53209774_A_C | 53209774 | 55132413 | rs2787486 | C | A | 0.3023 | −0.0361 | 0.9645 | 0.9787 |
| 17 | 17_53214654_C_T | 53214654 | 55137293 | rs244315 | T | C | 0.2795 | −0.0213 | 0.9789 | 0.9883 |
| 17 | 17_53306601_C_T | 53306601 | 55229240 | rs10515091 | T | C | 0.2360 | −0.0031 | 0.9969 | 0.9985 |
| 17 | 17_53324637_G_A | 53324637 | 55247276 | rs1106351 | A | G | 0.1643 | 0.0058 | 1.0058 | 1.0019 |
| 17 | 17_54154040_T_C | 54154040 | 56076679 | rs2877726 | C | T | 0.6450 | −0.0113 | 0.9888 | 0.9856 |
| 17 | 17_54336709_G_A | 54336709 | 56259348 | rs17821183 | A | G | 0.0536 | 0.0107 | 1.0108 | 1.0012 |
| 17 | 17_55451218_G_C | 55451218 | 57373857 | rs17221133 | C | G | 0.0525 | 0.0037 | 1.0037 | 1.0004 |
| 17 | 17_56494343_G_C | 56494343 | 58416982 | rs12950320 | C | G | 0.2290 | −0.0116 | 0.9885 | 0.9947 |
| 17 | 17_57561718_CAA | 57561718 | 59484357 | 17:57561718_ | C | CAA | 0.0343 | −0.0207 | 0.9795 | 0.9986 |
| | _C | | | CAA_C | | | | | | |
| 17 | 17_57870240_T_A | 57870240 | 59792879 | rs34678827 | A | T | 0.7974 | −0.0003 | 0.9997 | 0.9995 |
| 17 | 17_57929319_T_C | 57929319 | 59851958 | rs1292068 | C | T | 0.3023 | −0.0046 | 0.9954 | 0.9972 |
| 17 | 17_58051316_G_A | 58051316 | 59973955 | rs4968402 | A | G | 0.8424 | −0.0029 | 0.9971 | 0.9951 |
| 17 | 17_59547250_C_T | 59547250 | 61469889 | rs3785830 | T | C | 0.1062 | 0.0147 | 1.0148 | 1.0031 |
| 17 | 17_59765855_C_A | 59765855 | 61688494 | rs188327672 | A | C | 0.0314 | −0.0107 | 0.9894 | 0.9993 |
| 17 | 17_59963779_A_C | 59963779 | 61886418 | rs4968455 | C | A | 0.1646 | 0.0126 | 1.0127 | 1.0042 |
| 17 | 17_64886173_C_G | 64886173 | 66890056 | rs11079671 | G | C | 0.3071 | −0.0044 | 0.9956 | 0.9973 |
| 17 | 17_65261171_AAA | 65261171 | 67265055 | 17:65261171_ | A | AAAT | 0.5142 | −0.0065 | 0.9935 | 0.9933 |
| | TAAT_A | | | AAATAAT_A | | AAT | | | | |
| 17 | 17_65367292_T_C | 65367292 | 67371176 | rs6504501 | C | T | 0.2431 | 0.0125 | 1.0126 | 1.0061 |
| 17 | 17_66553479_T_C | 66553479 | 68557338 | rs11655740 | C | T | 0.0301 | −0.0027 | 0.9973 | 0.9998 |
| 17 | 17_66626373_A_C | 66626373 | 68630232 | rs11077678 | C | A | 0.4830 | −0.0050 | 0.9950 | 0.9952 |
| 17 | 17_66804173_G_A | 66804173 | 68808032 | rs2053585 | A | G | 0.6688 | −0.0033 | 0.9967 | 0.9956 |
| 17 | 17_66900804_C_T | 66900804 | 68904663 | rs36059695 | T | C | 0.0336 | −0.0135 | 0.9866 | 0.9991 |
| 17 | 17_68080125_G_C | 68080125 | 70083984 | rs8075605 | C | G | 0.7594 | 0.0066 | 1.0066 | 1.0101 |
| 17 | 17_69085396_G_T | 69085396 | 71089255 | rs8065642 | T | G | 0.4126 | −0.0037 | 0.9963 | 0.9970 |
| 17 | 17_70004689_T_C | 70004689 | 72008548 | rs2160441 | C | T | 0.5843 | 0.0051 | 1.0051 | 1.0060 |
| 17 | 17_70106428_T_C | 70106428 | 72110287 | rs1477066 | C | T | 0.6338 | 0.0077 | 1.0077 | 1.0098 |
| 17 | 17_70161079_G_A | 70161079 | 72164938 | rs11650866 | A | G | 0.1026 | −0.0059 | 0.9941 | 0.9988 |
| 17 | 17_70502866_T_C | 70502866 | 72506726 | rs9893321 | C | T | 0.0674 | −0.0005 | 0.9995 | 0.9999 |
| 17 | 17_70535819_A_A | 70535819 | 72539679 | rs150766779 | AC | A | 0.0655 | 0.0000 | 1.0000 | 1.0000 |
| | C | | | | | | | | | |
| 17 | 17_70605514_G_A | 70605514 | 72609375 | rs11651456 | A | G | 0.3628 | 0.0013 | 1.0013 | 1.0009 |
| 17 | 17_72570682_A_G | 72570682 | 74574543 | rs2655606 | G | A | 0.6700 | 0.0098 | 1.0098 | 1.0132 |
| 17 | 17_72774778_CTTT | 72774778 | 74778639 | 17:72774778_ | C | CTTT | 0.5737 | 0.0083 | 1.0083 | 1.0096 |
| | _C | | | CTTT_C | | | | | | |
| 17 | 17_75581812_C_T | 75581812 | 77585730 | rs11871602 | T | C | 0.0880 | 0.0000 | 1.0000 | 1.0000 |
| 17 | 17_76224762_T_A | 76224762 | 78228681 | rs744960 | A | T | 0.0822 | −0.0189 | 0.9813 | 0.9969 |
| 17 | 17_76247543_C_T | 76247543 | 78251462 | rs11868431 | T | C | 0.5183 | −0.0067 | 0.9933 | 0.9931 |
| 17 | 17_76371592_A_G | 76371592 | 78375511 | rs12601655 | G | A | 0.2020 | 0.0040 | 1.0040 | 1.0016 |
| 17 | 17_76386412_A_G | 76386412 | 78390331 | rs7503405 | G | A | 0.8805 | −0.0037 | 0.9963 | 0.9935 |
| 17 | 17_76492576_G_A | 76492576 | 217 | rs11657288 | A | G | 0.4078 | 0.0000 | 1.0000 | 1.0000 |
| 17 | 17_77781725_A_G | 77781725 | 217 | rs745570 | G | A | 0.5038 | −0.0122 | 0.9879 | 0.9878 |
| 17 | 17_77784268_T_C | 77784268 | 217 | rs2587505 | C | T | 0.4117 | −0.0057 | 0.9943 | 0.9953 |
| 17 | 17_77795012_C_T | 77795012 | 79821213 | rs8080723 | T | C | 0.0871 | −0.0094 | 0.9906 | 0.9984 |
| 17 | 17_77804840_C_T | 77804840 | 79831041 | rs9747992 | T | C | 0.0898 | 0.0000 | 1.0000 | 1.0000 |
| 17 | 17_77888930_A_G | 77888930 | 79915131 | rs1663186 | G | A | 0.0619 | −0.0166 | 0.9835 | 0.9980 |
| 17 | 17_78038139_C_T | 78038139 | 80064340 | rs56071031 | T | C | 0.1032 | 0.0215 | 1.0217 | 1.0045 |
| 17 | 17_78255053_T_C | 78255053 | 80281254 | rs62635847 | C | T | 0.3990 | −0.0053 | 0.9947 | 0.9958 |
| 17 | 17_80553220_C_G | 80553220 | 82595344 | rs11078002 | G | C | 0.7343 | −0.0057 | 0.9943 | 0.9917 |
| 17 | 17_80555477_T_C | 80555477 | 82597601 | rs62079673 | C | T | 0.5987 | −0.0046 | 0.9954 | 0.9945 |
| 17 | 17_80840588_A_G | 80840588 | 82882712 | rs9747095 | G | A | 0.4039 | 0.0029 | 1.0029 | 1.0023 |
| 17 | 17_80865406_G_C | 80865406 | 82907530 | rs2085615 | C | G | 0.3624 | 0.0003 | 1.0003 | 1.0002 |
| 17 | 17_80895487_AG_ | 80895487 | 82937611 | 17:80895487_ | A | AG | 0.0687 | 0.0069 | 1.0069 | 1.0010 |
| | A | | | AG_A | | | | | | |
| 17 | 17_80904310_C_T | 80904310 | 82946434 | rs898097 | T | C | 0.6529 | 0.0057 | 1.0057 | 1.0075 |
| 18 | 18_809911_G_A | 809911 | 809910 | rs9952321 | A | G | 0.0980 | 0.0043 | 1.0043 | 1.0008 |
| 18 | 18_862734_T_C | 862734 | 862733 | rs72871016 | C | T | 0.0997 | 0.0111 | 1.0112 | 1.0022 |
| 18 | 18_1197256_A_C | 1197256 | 1197255 | rs34516086 | C | A | 0.0242 | −0.0138 | 0.9863 | 0.9993 |
| 18 | 18_1630070_A_C | 1630070 | 1630069 | rs61229115 | C | A | 0.4179 | 0.0063 | 1.0063 | 1.0053 |
| 18 | 18_4293666_A_G | 4293666 | 4293666 | rs619143 | G | A | 0.6030 | 0.0000 | 1.0000 | 1.0000 |
| 18 | 18_7177489_C_T | 7177489 | 7177491 | rs9950440 | T | C | 0.1516 | −0.0154 | 0.9847 | 0.9954 |
| 18 | 18_8952017_G_A | 8952017 | 8952019 | rs62085542 | A | G | 0.0924 | −0.0211 | 0.9791 | 0.9961 |
| 18 | 18_8986040_G_A | 8986040 | 8986042 | rs57259999 | A | G | 0.2050 | 0.0027 | 1.0027 | 1.0011 |
| 18 | 18_8989816_C_T | 8989816 | 8989818 | rs12966593 | T | C | 0.1030 | −0.0030 | 0.9970 | 0.9994 |
| 18 | 18_9002582_G_A | 9002582 | 9002584 | rs965848 | A | G | 0.4379 | 0.0072 | 1.0072 | 1.0063 |
| 18 | 18_9008345_A_G | 9008345 | 9008347 | rs35743226 | G | A | 0.1243 | −0.0001 | 0.9999 | 1.0000 |
| 18 | 18_9484523_A_AT | 9484523 | 9484525 | | AT | A | 0.7180 | 0.0050 | 1.0050 | 1.0072 |
| 18 | 18_9531069_C_T | 9531069 | 9531071 | rs7226980 | T | C | 0.2016 | 0.0072 | 1.0072 | 1.0029 |
| 18 | 18_11066412_G_A | 11066412 | 11066413 | rs76722466 | A | G | 0.0102 | −0.0224 | 0.9778 | 0.9995 |
| 18 | 18_11696613_C_T | 11696613 | 11696614 | rs16976596 | T | C | 0.1379 | −0.0082 | 0.9918 | 0.9977 |
| 18 | 18_11750822_T_C | 11750822 | 11750823 | rs1005270 | C | T | 0.1598 | 0.0000 | 1.0000 | 1.0000 |
| 18 | 18_11757627_C_G | 11757627 | 11757628 | rs6505672 | G | C | 0.0309 | −0.0112 | 0.9889 | 0.9993 |
| 18 | 18_11771248_A_G | 11771248 | 11771249 | rs78297897 | G | A | 0.1212 | −0.0026 | 0.9974 | 0.9994 |
| 18 | 18_11807889_G_A | 11807889 | 11807890 | rs73946348 | A | G | 0.0849 | 0.0000 | 1.0000 | 1.0000 |
| 18 | 18_12937444_G_C | 12937444 | 12937445 | rs55796886 | C | G | 0.0287 | −0.0193 | 0.9809 | 0.9989 |
| 18 | 18_12956438_G_A | 12956438 | 12956439 | rs61480660 | A | G | 0.2243 | −0.0103 | 0.9898 | 0.9954 |
| 18 | 18_20634253_C_T | 20634253 | 23054290 | rs11665269 | T | C | 0.6403 | −0.0260 | 0.9743 | 0.9674 |
| 18 | 18_22117430_G_A | 22117430 | 24537466 | rs8093079 | A | G | 0.1839 | 0.0109 | 1.0110 | 1.0040 |
| 18 | 18_23164099_C_G | 23164099 | 25584135 | rs983864 | G | C | 0.4430 | 0.0013 | 1.0013 | 1.0012 |
| 18 | 18_23500096_T_C | 23500096 | 25920132 | rs7228892 | C | T | 0.6270 | 0.0031 | 1.0031 | 1.0039 |
| 18 | 18_23520441_T_A | 23520441 | 25940477 | rs4800697 | A | T | 0.5411 | 0.0095 | 1.0095 | 1.0104 |
| 18 | 18_24067931_A_G | 24067931 | 26487967 | rs2155759 | G | A | 0.5712 | −0.0086 | 0.9914 | 0.9902 |
| 18 | 18_24110979_G_A | 24110979 | 26531015 | rs62087056 | A | G | 0.1506 | 0.0117 | 1.0118 | 1.0035 |
| 18 | 18_24125857_T_C | 24125857 | 26545893 | rs1111207 | C | T | 0.4214 | 0.0070 | 1.0070 | 1.0059 |
| 18 | 18_24156018_C_T | 24156018 | 26576054 | rs7240205 | T | C | 0.6096 | −0.0005 | 0.9995 | 0.9994 |
| 18 | 18_24249279_G_A | 24249279 | 26669315 | rs7228610 | A | G | 0.6184 | −0.0032 | 0.9968 | 0.9961 |
| 18 | 18_24250541_G_C | 24250541 | 26670577 | rs78888860 | C | G | 0.0358 | 0.0124 | 1.0125 | 1.0009 |
| 18 | 18_24319537_A_G | 24319537 | 26739573 | rs582356 | G | A | 0.4067 | −0.0046 | 0.9954 | 0.9963 |
| 18 | 18_24321813_C_G | 24321813 | 26741849 | rs659378 | G | C | 0.5983 | −0.0037 | 0.9963 | 0.9956 |
| 18 | 18_24327257_A_G | 24327257 | 26747293 | rs139376186 | G | A | 0.0359 | −0.0029 | 0.9971 | 0.9998 |
| 18 | 18_24337424_C_G | 24337424 | 26757460 | rs527616 | G | C | 0.6205 | 0.0193 | 1.0195 | 1.0243 |
| 18 | 18_24453528_T_G | 24453528 | 26873564 | rs12969503 | G | T | 0.3019 | 0.0000 | 1.0000 | 1.0000 |
| 18 | 18_24497839_A_A | 24497839 | 26917875 | | ATT | A | 0.1133 | 0.0006 | 1.0006 | 1.0001 |
| | TT | | | | | | | | | |
| 18 | 18_24516214_C_T | 24516214 | 26936250 | rs10502480 | T | C | 0.1925 | −0.0044 | 0.9956 | 0.9983 |
| 18 | 18_24518050_AT_ | 24518050 | 26938086 | 18:24518050_ | A | AT | 0.2773 | −0.0195 | 0.9807 | 0.9893 |
| | A | | | AT_A | | | | | | |
| 18 | 18_24524941_A_T | 24524941 | 26944977 | rs12954099 | T | A | 0.2080 | −0.0018 | 0.9982 | 0.9993 |
| 18 | 18_24532012_G_A | 24532012 | 26952048 | rs335920 | A | G | 0.4333 | −0.0097 | 0.9903 | 0.9917 |
| 18 | 18_24575223_TA_T | 24575223 | 26995259 | 18:24575223_ | T | TA | 0.1534 | −0.0017 | 0.9983 | 0.9995 |
| | | | | TA_T | | | | | | |
| 18 | 18_24580869_C_T | 24580869 | 27000905 | rs74689581 | T | C | 0.0830 | 0.0041 | 1.0041 | 1.0007 |
| 18 | 18_24592146_C_T | 24592146 | 27012182 | rs1785519 | T | C | 0.4375 | −0.0072 | 0.9928 | 0.9937 |
| 18 | 18_24632639_G_A | 24632639 | 27052675 | rs4800783 | A | G | 0.7864 | −0.0016 | 0.9984 | 0.9975 |
| 18 | 18_24979859_TAC | 24979859 | 27399895 | 18:24979859_ | T | TACA | 0.4407 | 0.0066 | 1.0066 | 1.0058 |
| | AC_T | | | TACAC_T | | C | | | | |
| 18 | 18_25215126_A_G | 25215126 | 27635162 | rs62080926 | G | A | 0.4110 | −0.0010 | 0.9990 | 0.9992 |
| 18 | 18_25400338_GT_ | 25400338 | 27820374 | rs200577232 | G | GT | 0.6588 | 0.0000 | 1.0000 | 1.0000 |
| | G | | | | | | | | | |
| 18 | 18_25401204_AT_ | 25401204 | 27821240 | 18:25401204_ | A | AT | 0.6968 | 0.0071 | 1.0071 | 1.0100 |
| | A | | | AT_A | | | | | | |
| 18 | 18_25407918_A_A | 25407918 | 27827954 | rs36088218 | AT | A | 0.5683 | 0.0000 | 1.0000 | 1.0000 |
| | T | | | | | | | | | |
| 18 | 18_25444712_A_G | 25444712 | 27864748 | rs3924216 | G | A | 0.7271 | 0.0131 | 1.0132 | 1.0193 |
| 18 | 18_25458783_A_G | 25458783 | 27878819 | rs12386074 | G | A | 0.3590 | 0.0000 | 1.0000 | 1.0000 |
| 18 | 18_25460298_C_T | 25460298 | 27880334 | rs35149382 | T | C | 0.2476 | 0.0000 | 1.0000 | 1.0000 |
| 18 | 18_28936833_A_G | 28936833 | 31356870 | rs10502568 | G | A | 0.1432 | −0.0087 | 0.9913 | 0.9975 |
| 18 | 18_29138749_G_A | 29138749 | 31558786 | rs1791202 | A | G | 0.2257 | 0.0111 | 1.0112 | 1.0050 |
| 18 | 18_29981526_G_A | 29981526 | 32401563 | rs72931898 | A | G | 0.0474 | −0.0654 | 0.9367 | 0.9940 |
| 18 | 18_31713658_A_G | 31713658 | 34133694 | rs139406178 | G | A | 0.0181 | −0.0056 | 0.9944 | 0.9998 |
| 18 | 18_31768835_T_C | 31768835 | 34188871 | rs80264215 | C | T | 0.0260 | 0.0042 | 1.0042 | 1.0002 |
| 18 | 18_32606939_C_G | 32606939 | 35026975 | rs72950602 | G | C | 0.1732 | −0.0034 | 0.9966 | 0.9988 |
| 18 | 18_32633575_G_G | 32633575 | 35053611 | rs147017679 | GT | G | 0.0657 | −0.0323 | 0.9682 | 0.9958 |
| | T | | | | | | | | | |
| 18 | 18_32925069_AC_ | 32925069 | 35345105 | 18:32925069_ | A | AC | 0.0555 | −0.0066 | 0.9934 | 0.9993 |
| | A | | | AC_A | | | | | | |
| 18 | 18_33355868_T_G | 33355868 | 35775904 | rs72878547 | G | T | 0.0441 | −0.0194 | 0.9808 | 0.9983 |
| 18 | 18_33766007_G_C | 33766007 | 36186044 | rs607856 | C | G | 0.4912 | 0.0000 | 1.0000 | 1.0000 |
| 18 | 18_38314044_A_T | 38314044 | 40734080 | rs6507380 | T | A | 0.1582 | −0.0097 | 0.9903 | 0.9969 |
| 18 | 18_38534727_T_C | 38534727 | 40954763 | rs521589 | C | T | 0.8374 | 0.0076 | 1.0076 | 1.0128 |
| 18 | 18_42371193_G_A | 42371193 | 44791228 | rs113636799 | A | G | 0.0166 | 0.0000 | 1.0000 | 1.0000 |
| 18 | 18_42374231_G_G | 42374231 | 44794266 | rs147804054 | GTTTGAA | G | 0.0686 | −0.0078 | 0.9922 | 0.9989 |
| | TTTGAAAAAAA | | | | AAAAA | | | | | |
| 18 | 18_42411803_G_C | 42411803 | 44831838 | rs9954058 | C | G | 0.0717 | −0.0407 | 0.9601 | 0.9943 |
| 18 | 18_42880242_A_G | 42880242 | 45300277 | rs12456097 | G | A | 0.3301 | −0.0076 | 0.9924 | 0.9950 |
| 18 | 18_42888797_T_C | 42888797 | 45308832 | rs9952980 | C | T | 0.3519 | −0.0235 | 0.9768 | 0.9837 |
| 18 | 18_42974182_A_G | 42974182 | 45394217 | rs1362838 | G | A | 0.5465 | 0.0032 | 1.0032 | 1.0035 |
| 18 | 18_45044188_A_C | 45044188 | 47517817 | rs4939795 | C | A | 0.2754 | 0.0024 | 1.0024 | 1.0013 |
| 18 | 18_45055714_ATG | 45055714 | 47529343 | 18:45055714_ | A | ATGT | 0.4258 | 0.0069 | 1.0069 | 1.0059 |
| | TGTG_A | | | ATGTGTG_A | | GTG | | | | |
| 18 | 18_45642989_T_C | 45642989 | 48116618 | rs4479321 | C | T | 0.6957 | −0.0061 | 0.9939 | 0.9916 |
| 18 | 18_46451146_G_A | 46451146 | 48924776 | rs12956924 | A | G | 0.3241 | −0.0075 | 0.9925 | 0.9952 |
| 18 | 18_46453681_A_A | 46453681 | 48927311 | rs557304297 | AT | A | 0.4350 | −0.0009 | 0.9991 | 0.9992 |
| | T | | | | | | | | | |
| 18 | 18_46458227_A_C | 46458227 | 48931857 | rs9946510 | C | A | 0.3034 | 0.0022 | 1.0022 | 1.0013 |
| 18 | 18_48157969_G_C | 48157969 | 50631599 | rs8091238 | C | G | 0.6052 | −0.0089 | 0.9911 | 0.9893 |
| 18 | 18_48490805_G_A | 48490805 | 50964435 | rs646736 | A | G | 0.0205 | 0.0266 | 1.0270 | 1.0011 |
| 18 | 18_50371995_A_C | 50371995 | 52845625 | rs117929956 | C | A | 0.0303 | 0.0013 | 1.0013 | 1.0001 |
| 18 | 18_50436732_A_T | 50436732 | 52910362 | rs75666527 | T | A | 0.0398 | 0.0085 | 1.0085 | 1.0007 |
| 18 | 18_50681175_G_T | 50681175 | 53154805 | rs117276949 | T | G | 0.0247 | 0.0082 | 1.0082 | 1.0004 |
| 18 | 18_51766607_A_T | 51766607 | 54240237 | rs60287506 | T | A | 0.2291 | −0.0003 | 0.9997 | 0.9999 |
| 18 | 18_51847180_AT_ | 51847180 | 54320810 | 18:51847180_ | A | AT | 0.2389 | −0.0069 | 0.9931 | 0.9967 |
| | A | | | AT_A | | | | | | |
| 18 | 18_53605004_G_C | 53605004 | 55937773 | rs80129237 | C | G | 0.0172 | −0.0236 | 0.9767 | 0.9992 |
| 18 | 18_54060289_T_C | 54060289 | 56393058 | rs302039 | C | T | 0.3670 | 0.0003 | 1.0003 | 1.0002 |
| 18 | 18_54200266_T_C | 54200266 | 56533035 | rs663220 | C | T | 0.3957 | 0.0066 | 1.0066 | 1.0052 |
| 18 | 18_55404115_C_T | 55404115 | 57736883 | rs9676158 | T | C | 0.0671 | −0.0126 | 0.9875 | 0.9983 |
| 18 | 18_56058384_A_C | 56058384 | 58391152 | rs8098126 | C | A | 0.5435 | −0.0102 | 0.9899 | 0.9890 |
| 18 | 18_56061323_A_G | 56061323 | 58394091 | rs8086848 | G | A | 0.5736 | −0.0044 | 0.9956 | 0.9950 |
| 18 | 18_56958687_C_T | 56958687 | 59291455 | rs8082941 | T | C | 0.3554 | 0.0041 | 1.0041 | 1.0029 |
| 18 | 18_57056766_G_T | 57056766 | 59389534 | rs8087926 | T | G | 0.4013 | 0.0000 | 1.0000 | 1.0000 |
| 18 | 18_57898005_AT_ | 57898005 | 60230772 | rs35407057 | ATT | AT | 0.2624 | −0.0007 | 0.9993 | 0.9996 |
| | ATT | | | | | | | | | |
| 18 | 18_60047489_G_A | 60047489 | 62380256 | rs9948182 | A | G | 0.3344 | −0.0045 | 0.9955 | 0.9970 |
| 18 | 18_60296752_C_T | 60296752 | 62629519 | rs668519 | T | C | 0.8361 | 0.0007 | 1.0007 | 1.0012 |
| 18 | 18_60393565_T_G | 60393565 | 62726332 | rs189749253 | G | T | 0.0540 | −0.0189 | 0.9813 | 0.9980 |
| 18 | 18_60826343_G_A | 60826343 | 63159110 | rs4940572 | A | G | 0.1475 | 0.0077 | 1.0077 | 1.0023 |
| 18 | 18_60835170_A_T | 60835170 | 63167937 | rs8094630 | T | A | 0.1506 | 0.0053 | 1.0053 | 1.0016 |
| 18 | 18_60845291_T_C | 60845291 | 63178058 | rs4940575 | C | T | 0.1644 | 0.0035 | 1.0035 | 1.0012 |
| 18 | 18_66888054_A_G | 66888054 | 69220817 | rs17080623 | G | A | 0.2338 | 0.0074 | 1.0074 | 1.0035 |
| 18 | 18_66888819_T_C | 66888819 | 69221582 | rs615767 | C | T | 0.6311 | −0.0043 | 0.9957 | 0.9946 |
| 18 | 18_66889796_C_G | 66889796 | 69222559 | rs8093619 | G | C | 0.0513 | 0.0162 | 1.0163 | 1.0017 |
| 18 | 18_74194978_T_C | 74194978 | 76483022 | rs78380260 | C | T | 0.1186 | −0.0179 | 0.9823 | 0.9958 |
| 18 | 18_74197470_T_C | 74197470 | 76485514 | rs58045795 | C | T | 0.6370 | 0.0014 | 1.0014 | 1.0018 |
| 18 | 18_74199721_C_T | 74199721 | 76487765 | rs10460104 | T | C | 0.1152 | −0.0071 | 0.9929 | 0.9984 |
| 18 | 18_74967894_T_C | 74967894 | 77255938 | rs9959924 | C | T | 0.0760 | 0.0088 | 1.0088 | 1.0013 |
| 18 | 18_75006480_A_G | 75006480 | 77294524 | rs11659581 | G | A | 0.0786 | 0.0034 | 1.0034 | 1.0005 |
| 18 | 18_75105794_C_T | 75105794 | 77393838 | rs78446678 | T | C | 0.0332 | −0.0337 | 0.9669 | 0.9978 |
| 18 | 18_75618836_G_A | 75618836 | 77906880 | rs76184407 | A | G | 0.0225 | −0.0245 | 0.9758 | 0.9989 |
| 18 | 18_75912922_A_G | 75912922 | 78152922 | rs4798896 | G | A | 0.3497 | −0.0131 | 0.9870 | 0.9909 |
| 18 | 18_76342086_A_G | 76342086 | 78582086 | rs75819391 | G | A | 0.0192 | −0.0081 | 0.9919 | 0.9997 |
| 19 | 19_1396952_T_C | 1396952 | 1396953 | rs266813 | C | T | 0.0369 | −0.0335 | 0.9671 | 0.9976 |
| 19 | 19_2211050_C_T | 2211050 | 2211051 | rs2301800 | T | C | 0.4279 | 0.0164 | 1.0165 | 1.0142 |
| 19 | 19_4920414_A_G | 4920414 | 4920402 | rs12972366 | G | A | 0.2567 | −0.0043 | 0.9957 | 0.9978 |
| 19 | 19_4922095_A_G | 4922095 | 4922083 | rs2656922 | G | A | 0.2365 | −0.0056 | 0.9944 | 0.9974 |
| 19 | 19_4939092_A_AT | 4939092 | 4939080 | rs71170881 | AT | A | 0.2354 | −0.0003 | 0.9997 | 0.9999 |
| 19 | 19_5836856_AAAA | 5836856 | 5836845 | 19:5836856_A | A | AAAA | 0.2760 | −0.0052 | 0.9948 | 0.9971 |
| | T_A | | | AAAT_A | | T | | | | |
| 19 | 19_5840619_C_T | 5840619 | 5840608 | rs708686 | T | C | 0.3104 | −0.0026 | 0.9974 | 0.9984 |
| 19 | 19_8367525_G_C | 8367525 | 8302641 | rs2227288 | C | G | 0.1209 | 0.0027 | 1.0027 | 1.0007 |
| 19 | 19_9060605_G_A | 9060605 | 8949929 | rs2194168 | A | G | 0.8888 | −0.0111 | 0.9890 | 0.9805 |
| 19 | 19_11342703_A_G | 11342703 | 11232027 | rs12979813 | G | A | 0.1832 | −0.0018 | 0.9982 | 0.9993 |
| 19 | 19_12069444_T_G | 12069444 | 11958629 | rs279273 | G | T | 0.0806 | 0.0222 | 1.0224 | 1.0036 |
| 19 | 19_13070749_G_T | 13070749 | 12959935 | rs12984830 | T | G | 0.6454 | −0.0018 | 0.9982 | 0.9977 |
| 19 | 19_13094298_C_T | 13094298 | 12983484 | rs7409148 | T | C | 0.8202 | −0.0037 | 0.9963 | 0.9940 |
| 19 | 19_13158277_T_C | 13158277 | 13047463 | rs78269692 | C | T | 0.0510 | 0.0386 | 1.0394 | 1.0040 |
| 19 | 19_13171576_T_TG | 13171576 | 13060762 | rs35712689 | TG | T | 0.0606 | −0.0091 | 0.9909 | 0.9989 |
| 19 | 19_13208001_GTTT | 13208001 | 13097187 | | GTTTTT | GTTT | 0.0323 | −0.0001 | 0.9999 | 1.0000 |
| | _GTTTTT | | | | | | | | | |
| 19 | 19_13249921_G_T | 13249921 | 13139107 | rs117922601 | T | G | 0.0513 | 0.0056 | 1.0056 | 1.0006 |
| 19 | 19_13954571_G_A | 13954571 | 13843757 | rs2594714 | A | G | 0.2285 | −0.0027 | 0.9973 | 0.9988 |
| 19 | 19_13956663_T_G | 13956663 | 13845849 | rs4271693 | G | T | 0.2547 | 0.0000 | 1.0000 | 1.0000 |
| 19 | 19_13959052_C_CC | 13959052 | 13848238 | rs5827214 | CCCG | C | 0.2543 | 0.0000 | 1.0000 | 1.0000 |
| | CG | | | | | | | | | |
| 19 | 19_14629171_C_T | 14629171 | 14518359 | rs34966794 | T | C | 0.0820 | −0.0042 | 0.9958 | 0.9993 |
| 19 | 19_17117341_GA_ | 17117341 | 17006531 | 19:17117341_ | G | GA | 0.1921 | −0.0039 | 0.9961 | 0.9985 |
| | G | | | GA_G | | | | | | |
| 19 | 19_17120477_G_A | 17120477 | 17009667 | rs113926826 | A | G | 0.0958 | −0.0045 | 0.9955 | 0.9991 |
| 19 | 19_17148096_C_T | 17148096 | 17037286 | rs755088 | T | C | 0.3747 | 0.0041 | 1.0041 | 1.0031 |
| 19 | 19_17210615_G_A | 17210615 | 17099805 | rs7249698 | A | G | 0.3677 | −0.0073 | 0.9927 | 0.9947 |
| 19 | 19_17332663_A_C | 17332663 | 17221854 | rs7253172 | C | A | 0.3572 | 0.0000 | 1.0000 | 1.0000 |
| 19 | 19_17383602_C_T | 17383602 | 17272793 | rs62126227 | T | C | 0.4212 | −0.0002 | 0.9998 | 0.9998 |
| 19 | 19_17393925_C_A | 17393925 | 17283116 | rs56069439 | A | C | 0.2958 | 0.0121 | 1.0122 | 1.0072 |
| 19 | 19_17397501_T_G | 17397501 | 17286692 | rs1465582 | G | T | 0.3998 | −0.0097 | 0.9903 | 0.9923 |
| 19 | 19_17404269_GGG | 17404269 | 17293460 | 19:17404269_ | G | GGGG | 0.5129 | 0.0000 | 1.0000 | 1.0000 |
| | GCGGGGGT_G | | | GGGGCGGGG | | CGGG | | | | |
| | | | | GT_G | | GGT | | | | |
| 19 | 19_17913671_T_A | 17913671 | 17802862 | rs424788 | A | T | 0.2346 | −0.0007 | 0.9993 | 0.9997 |
| 19 | 19_18217776_ATTT | 18217776 | 18106966 | 19:18217776_ | A | ATTT | 0.4258 | 0.0115 | 1.0116 | 1.0099 |
| | TTTTTTTTTTTTT_A | | | ATTTTTTTTTTT | | TTTT | | | | |
| | | | | TTTTT_A | | TTTT | | | | |
| | | | | | | TTTT | | | | |
| | | | | | | T | | | | |
| 19 | 19_18569492_C_T | 18569492 | 18458682 | rs10164323 | T | C | 0.3481 | −0.0491 | 0.9521 | 0.9669 |
| 19 | 19_18589943_C_T | 18589943 | 18479133 | rs11670056 | T | C | 0.0823 | 0.0000 | 1.0000 | 1.0000 |
| 19 | 19_18637610_C_T | 18637610 | 18526800 | rs4808814 | T | C | 0.3254 | −0.0012 | 0.9988 | 0.9992 |
| 19 | 19_18695690_C_T | 18695690 | 18584880 | rs144474740 | T | C | 0.0681 | 0.0000 | 1.0000 | 1.0000 |
| 19 | 19_19347367_G_A | 19347367 | 19236558 | rs77927505 | A | G | 0.0630 | 0.0054 | 1.0054 | 1.0007 |
| 19 | 19_19347538_CTT_ | 19347538 | 19236729 | 19:19347538_ | C | CTT | 0.0369 | 0.0168 | 1.0169 | 1.0013 |
| | C | | | CTT_C | | | | | | |
| 19 | 19_19517054_C_C | 19517054 | 19406245 | rs140702307 | CGGGCG | C | 0.3537 | 0.0017 | 1.0017 | 1.0012 |
| | GGGCG | | | | | | | | | |
| 19 | 19_19577215_C_G | 19577215 | 19466406 | rs4808961 | G | C | 0.3673 | 0.0189 | 1.0191 | 1.0141 |
| 19 | 19_19765269_G_C | 19765269 | 19654460 | rs892036 | C | G | 0.7114 | 0.0103 | 1.0104 | 1.0148 |
| 19 | 19_19856490_G_A | 19856490 | 19745681 | rs8099910 | A | G | 0.3558 | −0.0032 | 0.9968 | 0.9977 |
| 19 | 19_21439532_G_A | 21439532 | 21256730 | rs76145660 | A | G | 0.0165 | 0.0253 | 1.0256 | 1.0008 |
| 19 | 19_29565686_C_G | 29565686 | 29074779 | rs71363948 | G | C | 0.0166 | −0.0068 | 0.9932 | 0.9998 |
| 19 | 19_30230785_A_T | 30230785 | 29739878 | rs12976223 | T | A | 0.6343 | −0.0098 | 0.9902 | 0.9877 |
| 19 | 19_30273316_G_A | 30273316 | 29782409 | rs73552114 | A | G | 0.0433 | −0.0054 | 0.9946 | 0.9995 |
| 19 | 19_30344206_CT_C | 30344206 | 29853299 | 19:30344206_ | C | CT | 0.2769 | 0.0016 | 1.0016 | 1.0009 |
| | | | | CT_C | | | | | | |
| 19 | 19_30350666_T_C | 30350666 | 29859759 | rs17599667 | C | T | 0.2766 | 0.0000 | 1.0000 | 1.0000 |
| 19 | 19_30365074_C_A | 30365074 | 29874167 | rs62104509 | A | C | 0.2816 | 0.0000 | 1.0000 | 1.0000 |
| 19 | 19_33521150_A_G | 33521150 | 33030244 | rs73039428 | G | A | 0.0608 | 0.0139 | 1.0140 | 1.0017 |
| 19 | 19_34208223_A_G | 34208223 | 33717318 | rs157726 | G | A | 0.5528 | 0.0034 | 1.0034 | 1.0038 |
| 19 | 19_35099785_T_G | 35099785 | 34608880 | rs78034480 | G | T | 0.0263 | 0.0097 | 1.0097 | 1.0005 |
| 19 | 19_36001912_T_C | 36001912 | 35511010 | rs75264675 | C | T | 0.0217 | 0.0077 | 1.0077 | 1.0003 |
| 19 | 19_37833311_ATTT | 37833311 | 37342409 | rs756411001 | A | ATTT | 0.2660 | 0.0018 | 1.0018 | 1.0010 |
| | TTT_A | | | | | TTT | | | | |
| 19 | 19_38336063_C_C | 38336063 | 37845423 | rs572041567 | CA | C | 0.2859 | 0.0047 | 1.0047 | 1.0027 |
| | A | | | | | | | | | |
| 19 | 19_38399256_GT_ | 38399256 | 37908616 | 19:38399256_ | G | GT | 0.1790 | 0.0054 | 1.0054 | 1.0019 |
| | G | | | GT_G | | | | | | |
| 19 | 19_38704921_A_C | 38704921 | 38214281 | rs12610840 | C | A | 0.2005 | 0.0010 | 1.0010 | 1.0004 |
| 19 | 19_38705656_A_A | 38705656 | 38215016 | rs200548028 | AG | A | 0.1891 | 0.0050 | 1.0050 | 1.0019 |
| | G | | | | | | | | | |
| 19 | 19_39459994_G_A | 39459994 | 38969354 | rs4803141 | A | G | 0.3635 | −0.0072 | 0.9928 | 0.9948 |
| 19 | 19_41564691_TA_T | 41564691 | 41058786 | 19:41564691_ | T | TA | 0.0318 | 0.0158 | 1.0159 | 1.0010 |
| | | | | TA_T | | | | | | |
| 19 | 19_42345760_C_C | 42345760 | 41841665 | rs587607752 | CA | C | 0.4612 | −0.0073 | 0.9927 | 0.9933 |
| | A | | | | | | | | | |
| 19 | 19_43204784_A_G | 43204784 | 42700632 | rs1807350 | G | A | 0.0795 | 0.0030 | 1.0030 | 1.0005 |
| 19 | 19_43979589_G_T | 43979589 | 43475437 | rs1055099 | T | G | 0.4155 | −0.0064 | 0.9936 | 0.9947 |
| 19 | 19_43986874_G_C | 43986874 | 43482722 | rs143888974 | C | G | 0.0555 | −0.0102 | 0.9899 | 0.9989 |
| 19 | 19_44283031_T_C | 44283031 | 43778879 | rs56681946 | C | T | 0.3519 | 0.0289 | 1.0293 | 1.0207 |
| 19 | 19_44283447_C_T | 44283447 | 43779295 | rs12609846 | T | C | 0.5667 | −0.0034 | 0.9966 | 0.9962 |
| 19 | 19_44284315_C_G | 44284315 | 43780163 | rs12461670 | G | C | 0.1461 | −0.0031 | 0.9969 | 0.9991 |
| 19 | 19_44374631_A_G | 44374631 | 43870479 | rs11673020 | G | A | 0.5150 | −0.0104 | 0.9897 | 0.9894 |
| 19 | 19_44377614_G_A | 44377614 | 43873462 | rs76483217 | A | G | 0.0218 | 0.0000 | 1.0000 | 1.0000 |
| 19 | 19_44427764_G_T | 44427764 | 43923612 | rs450880 | T | G | 0.1704 | −0.0100 | 0.9900 | 0.9966 |
| 19 | 19_44481934_A_G | 44481934 | 43977782 | rs79428339 | G | A | 0.0182 | 0.0000 | 1.0000 | 1.0000 |
| 19 | 19_45134682_A_C | 45134682 | 44631381 | rs62119267 | C | A | 0.0187 | 0.0034 | 1.0034 | 1.0001 |
| 19 | 19_45191450_GTTT | 45191450 | 44688178 | 19:45191450_ | G | GTTT | 0.1169 | −0.0184 | 0.9818 | 0.9957 |
| | TC_G | | | GTTTTC_G | | TC | | | | |
| 19 | 19_45198477_C_T | 45198477 | 44695207 | rs10422568 | T | C | 0.0432 | 0.0196 | 1.0198 | 1.0017 |
| 19 | 19_45475713_A_G | 45475713 | 44972456 | rs204482 | G | A | 0.4095 | 0.0028 | 1.0028 | 1.0023 |
| 19 | 19_46134289_G_C | 46134289 | 45631031 | rs57724625 | C | G | 0.2434 | 0.0002 | 1.0002 | 1.0001 |
| 19 | 19_46164766_G_A | 46164766 | 45661508 | rs74174203 | A | G | 0.5407 | 0.0000 | 1.0000 | 1.0000 |
| 19 | 19_46166073_T_C | 46166073 | 45662815 | rs4399645 | C | T | 0.6074 | −0.0096 | 0.9904 | 0.9884 |
| 19 | 19_46182741_G_G | 46182741 | 45679483 | rs572702775 | GA | G | 0.2722 | −0.0062 | 0.9938 | 0.9966 |
| | A | | | | | | | | | |
| 19 | 19_46183586_C_T | 46183586 | 45680328 | rs61373376 | T | C | 0.1691 | 0.0042 | 1.0042 | 1.0014 |
| 19 | 19_46199363_G_A | 46199363 | 45696105 | rs74889068 | A | G | 0.1550 | 0.0018 | 1.0018 | 1.0006 |
| 19 | 19_46238239_C_T | 46238239 | 45734981 | rs62111736 | T | C | 0.0140 | −0.0265 | 0.9738 | 0.9993 |
| 19 | 19_46274624_G_A | 46274624 | 45771366 | rs146680240 | A | G | 0.0155 | 0.0416 | 1.0425 | 1.0013 |
| 19 | 19_47298001_T_C | 47298001 | 46794744 | rs57753335 | C | T | 0.2228 | −0.0120 | 0.9881 | 0.9947 |
| 19 | 19_49634592_ATTT | 49634592 | 49131335 | rs763666162 | A | ATTT | 0.6530 | 0.0028 | 1.0028 | 1.0037 |
| | _A | | | | | | | | | |
| 19 | 19_49784891_A_G | 49784891 | 49281634 | rs354002 | G | A | 0.5218 | 0.0035 | 1.0035 | 1.0037 |
| 19 | 19_54421924_G_A | 54421924 | 53918670 | rs28462357 | A | G | 0.0149 | 0.0301 | 1.0306 | 1.0009 |
| 19 | 19_54654779_C_T | 54654779 | 54151042 | rs36660 | T | C | 0.2813 | −0.0027 | 0.9973 | 0.9985 |
| 19 | 19_54665970_C_T | 54665970 | 54162234 | rs36655 | T | C | 0.3269 | −0.0086 | 0.9914 | 0.9944 |
| 19 | 19_54672776_CT_C | 54672776 | 54169029 | 19:54672776_ | C | CT | 0.3141 | −0.0028 | 0.9972 | 0.9982 |
| | | | | CT_C | | | | | | |
| 19 | 19_55816678_C_T | 55816678 | 55305310 | rs1172821 | T | C | 0.3626 | −0.0060 | 0.9940 | 0.9957 |
| 19 | 19_55837531_G_A | 55837531 | 55326163 | rs2384686 | A | G | 0.3524 | −0.0074 | 0.9926 | 0.9948 |
| 19 | 19_56060541_C_G | 56060541 | 55549175 | rs7247512 | G | C | 0.1831 | −0.0097 | 0.9903 | 0.9965 |
| 20 | 20_1932164_G_A | 1932164 | 1951518 | rs6105964 | A | G | 0.1038 | −0.0036 | 0.9964 | 0.9993 |
| 20 | 20_3679816_G_A | 3679816 | 3699169 | rs45587432 | A | G | 0.0279 | −0.0316 | 0.9689 | 0.9983 |
| 20 | 20_5746348_A_G | 5746348 | 5765702 | rs6107689 | G | A | 0.3868 | −0.0129 | 0.9872 | 0.9901 |
| 20 | 20_5762289_A_AT | 5762289 | 5781643 | rs147459548 | AT | A | 0.2528 | −0.0026 | 0.9974 | 0.9987 |
| 20 | 20_5948227_G_A | 5948227 | 5967581 | rs16991615 | A | G | 0.0628 | 0.0233 | 1.0236 | 1.0030 |
| 20 | 20_6046437_A_T | 6046437 | 6065791 | rs745296 | T | A | 0.5945 | 0.0011 | 1.0011 | 1.0013 |
| 20 | 20_6047152_T_C | 6047152 | 6066506 | rs1884650 | C | T | 0.3136 | 0.0039 | 1.0039 | 1.0025 |
| 20 | 20_6059635_T_C | 6059635 | 6078988 | rs2236147 | C | T | 0.2043 | 0.0052 | 1.0052 | 1.0021 |
| 20 | 20_6427312_C_A | 6427312 | 6446665 | rs74460586 | A | C | 0.0115 | −0.0139 | 0.9862 | 0.9997 |
| 20 | 20_6670853_C_T | 6670853 | 6690206 | rs2876050 | T | C | 0.5484 | 0.0012 | 1.0012 | 1.0013 |
| 20 | 20_6821115_C_T | 6821115 | 6840468 | rs1392626 | T | C | 0.0914 | 0.0150 | 1.0151 | 1.0028 |
| 20 | 20_7876255_A_G | 7876255 | 7895608 | rs6117980 | G | A | 0.7022 | 0.0051 | 1.0051 | 1.0072 |
| 20 | 20_10652959_G_A | 10652959 | 10672311 | rs2235810 | A | G | 0.1140 | −0.0018 | 0.9982 | 0.9996 |
| 20 | 20_11177529_G_A | 11177529 | 11196881 | rs2207412 | A | G | 0.4969 | 0.0007 | 1.0007 | 1.0007 |
| 20 | 20_11186652_C_T | 11186652 | 11206004 | rs6078099 | T | C | 0.3669 | 0.0089 | 1.0089 | 1.0066 |
| 20 | 20_11379842_T_C | 11379842 | 11399194 | rs1154723 | C | T | 0.9483 | 0.0365 | 1.0372 | 1.0718 |
| 20 | 20_11414962_CA_ | 11414962 | 11434314 | | C | CA | 0.0913 | 0.0000 | 1.0000 | 1.0000 |
| | C | | | | | | | | | |
| 20 | 20_11461558_A_G | 11461558 | 11480910 | rs6078210 | G | A | 0.7071 | 0.0000 | 1.0000 | 1.0000 |
| 20 | 20_11502618_A_A | 11502618 | 11521970 | rs141526427 | AAC | A | 0.2463 | −0.0080 | 0.9920 | 0.9961 |
| | AC | | | | | | | | | |
| 20 | 20_11530650_G_A | 11530650 | 11550002 | rs2423573 | A | G | 0.2664 | −0.0033 | 0.9967 | 0.9982 |
| 20 | 20_11612323_A_T | 11612323 | 11631675 | rs75314238 | T | A | 0.0583 | −0.0058 | 0.9942 | 0.9993 |
| 20 | 20_13825007_AGT | 13825007 | 13844361 | 20:13825007_ | A | AGTG | 0.7515 | 0.0078 | 1.0078 | 1.0118 |
| | GT_A | | | AGTGT_A | | T | | | | |
| 20 | 20_16625607_T_A | 16625607 | 16644962 | rs967603 | A | T | 0.2126 | 0.0066 | 1.0066 | 1.0028 |
| 20 | 20_16626691_G_C | 16626691 | 16646046 | rs2184501 | C | G | 0.1367 | 0.0017 | 1.0017 | 1.0005 |
| 20 | 20_16741090_C_G | 16741090 | 16760445 | rs4814552 | G | C | 0.4183 | −0.0007 | 0.9993 | 0.9994 |
| 20 | 20_19186447_G_C | 19186447 | 19205803 | rs78309861 | C | G | 0.0239 | −0.0115 | 0.9886 | 0.9995 |
| 20 | 20_19199808_A_C | 19199808 | 19219164 | rs149337215 | C | A | 0.0248 | −0.0313 | 0.9692 | 0.9985 |
| 20 | 20_19239386_A_G | 19239386 | 19258742 | rs6035268 | G | A | 0.5804 | 0.0090 | 1.0090 | 1.0105 |
| 20 | 20_19547213_C_T | 19547213 | 19566569 | rs2328411 | T | C | 0.7711 | −0.0154 | 0.9847 | 0.9766 |
| 20 | 20_19721433_G_A | 19721433 | 19740789 | rs56236053 | A | G | 0.3377 | 0.0046 | 1.0046 | 1.0031 |
| 20 | 20_19743404_A_T | 19743404 | 19762760 | rs6081740 | T | A | 0.4312 | 0.0008 | 1.0008 | 1.0007 |
| 20 | 20_20570540_A_T | 20570540 | 20589896 | rs6075678 | T | A | 0.0422 | 0.0010 | 1.0010 | 1.0001 |
| 20 | 20_20703519_A_A | 20703519 | 20722876 | rs373593768 | AAAT | A | 0.0385 | 0.0215 | 1.0217 | 1.0017 |
| | AAT | | | | | | | | | |
| 20 | 20_20895989_T_C | 20895989 | 20915346 | rs117558716 | C | T | 0.0132 | −0.0170 | 0.9831 | 0.9996 |
| 20 | 20_30437522_A_G | 30437522 | 31849719 | rs3746599 | G | A | 0.4158 | −0.0059 | 0.9941 | 0.9951 |
| 20 | 20_31984124_G_A | 31984124 | 33396318 | rs6059298 | A | G | 0.1677 | −0.0072 | 0.9928 | 0.9976 |
| 20 | 20_32381337_A_G | 32381337 | 33793531 | rs2747567 | G | A | 0.5880 | −0.0028 | 0.9972 | 0.9967 |
| 20 | 20_32468680_CT_C | 32468680 | 33880874 | 20:32468680_ | C | CT | 0.1395 | −0.0025 | 0.9975 | 0.9993 |
| | | | | CT_C | | | | | | |
| 20 | 20_34472105_C_T | 34472105 | 35884183 | rs3787173 | T | C | 0.0996 | 0.0145 | 1.0146 | 1.0029 |
| 20 | 20_39522409_T_C | 39522409 | 40893769 | rs6124289 | C | T | 0.4092 | 0.0036 | 1.0036 | 1.0030 |
| 20 | 20_40014674_G_A | 40014674 | 41386034 | rs4810315 | A | G | 0.3348 | 0.0013 | 1.0013 | 1.0009 |
| 20 | 20_40014981_TTG | 40014981 | 41386341 | 20:40014981_ | T | TTGG | 0.1565 | −0.0065 | 0.9935 | 0.9980 |
| | GG_T | | | TTGGG_T | | G | | | | |
| 20 | 20_41613706_C_G | 41613706 | 42985066 | rs6030585 | G | C | 0.7928 | 0.0102 | 1.0103 | 1.0163 |
| 20 | 20_43374159_AGG | 43374159 | 44745518 | 20:43374159_ | A | AGGA | 0.0337 | 0.0019 | 1.0019 | 1.0001 |
| | AG_A | | | AGGAG_A | | G | | | | |
| 20 | 20_43374227_C_A | 43374227 | 44745586 | rs6031778 | A | C | 0.0297 | 0.0056 | 1.0056 | 1.0003 |
| 20 | 20_44220832_G_A | 44220832 | 45592193 | rs6130867 | A | G | 0.0640 | 0.0230 | 1.0233 | 1.0030 |
| 20 | 20_47078957_A_G | 47078957 | 48450711 | rs926693 | G | A | 0.4750 | 0.0035 | 1.0035 | 1.0033 |
| 20 | 20_48946692_A_G | 48946692 | 50330155 | rs12479601 | G | A | 0.1787 | 0.0095 | 1.0095 | 1.0034 |
| 20 | 20_49002504_G_T | 49002504 | 50385967 | rs113609374 | T | G | 0.0256 | 0.0006 | 1.0006 | 1.0000 |
| 20 | 20_49023796_C_A | 49023796 | 50407259 | rs6063510 | A | C | 0.7557 | 0.0000 | 1.0000 | 1.0000 |
| 20 | 20_49031318_G_T | 49031318 | 50414781 | rs6020487 | T | G | 0.2672 | 0.0046 | 1.0046 | 1.0025 |
| 20 | 20_49058736_C_T | 49058736 | 50442199 | rs6095944 | T | C | 0.4503 | 0.0000 | 1.0000 | 1.0000 |
| 20 | 20_49061380_TA_T | 49061380 | 50444843 | 20:49061380_ | T | TA | 0.4591 | −0.0023 | 0.9977 | 0.9979 |
| | | | | TA_T | | | | | | |
| 20 | 20_49072666_G_A | 49072666 | 50456129 | rs118137582 | A | G | 0.0613 | 0.0085 | 1.0085 | 1.0010 |
| 20 | 20_49240476_C_T | 49240476 | 50623939 | rs2426172 | T | C | 0.0340 | 0.0126 | 1.0127 | 1.0009 |
| 20 | 20_49392744_C_T | 49392744 | 50776207 | rs151002938 | T | C | 0.0477 | 0.0000 | 1.0000 | 1.0000 |
| 20 | 20_49394206_TC_T | 49394206 | 50777669 | 20:49394206_ | T | TC | 0.0158 | −0.0162 | 0.9839 | 0.9995 |
| | | | | TC_T | | | | | | |
| 20 | 20_49428692_T_C | 49428692 | 50812155 | rs62202841 | C | T | 0.2844 | −0.0062 | 0.9938 | 0.9965 |
| 20 | 20_50788244_T_G | 50788244 | 52171705 | rs1964133 | G | T | 0.5109 | −0.0003 | 0.9997 | 0.9997 |
| 20 | 20_51200352_T_G | 51200352 | 52583813 | rs17733099 | G | T | 0.1805 | −0.0008 | 0.9992 | 0.9997 |
| 20 | 20_52296849_G_A | 52296849 | 53680310 | rs13039563 | A | G | 0.2400 | 0.0204 | 1.0206 | 1.0099 |
| 20 | 20_52298228_G_A | 52298228 | 53681689 | rs6097525 | A | G | 0.3126 | 0.0000 | 1.0000 | 1.0000 |
| 20 | 20_52298514_A_G | 52298514 | 53681975 | rs13039994 | G | A | 0.2366 | 0.0000 | 1.0000 | 1.0000 |
| 20 | 20_52300359_C_T | 52300359 | 53683820 | rs6126919 | T | C | 0.4305 | −0.0014 | 0.9986 | 0.9988 |
| 20 | 20_52573971_T_G | 52573971 | 53957432 | rs35575210 | G | T | 0.1807 | 0.0209 | 1.0211 | 1.0076 |
| 20 | 20_53229113_C_A | 53229113 | 54612574 | rs35338030 | A | C | 0.1845 | −0.0090 | 0.9910 | 0.9967 |
| 20 | 20_54963164_C_CC | 54963164 | 56388108 | 20:54963164_ | CCATGTG | C | 0.0147 | 0.0056 | 1.0056 | 1.0002 |
| | ATGTGGGTTTATAA | | | C_CCATGTGG | GGTTTAT | | | | | |
| | ATGTGAATGAGATT | | | GTTTATAAATG | AAATGTG | | | | | |
| | ACAGATTATTCAATT | | | TGAATGAGAT | AATGAGA | | | | | |
| | TAGACAAGACCAAC | | | TACAGATTATT | TTACAGA | | | | | |
| | CTG | | | CAATTTAGACA | TTATTCA | | | | | |
| | | | | AGACCAACCT | ATTTAGA | | | | | |
| | | | | G | CAAGACC | | | | | |
| | | | | | AACCTG | | | | | |
| 20 | 20_58247236_T_C | 58247236 | 59672181 | rs860973 | C | T | 0.8214 | −0.0062 | 0.9938 | 0.9899 |
| 20 | 20_58248796_G_C | 58248796 | 59673741 | rs73140881 | C | G | 0.1653 | 0.0035 | 1.0035 | 1.0012 |
| 20 | 20_59044356_G_A | 59044356 | 60469298 | rs348798 | A | G | 0.6481 | 0.0093 | 1.0093 | 1.0121 |
| 20 | 20_59056866_T_C | 59056866 | 60481808 | rs348787 | C | T | 0.5891 | 0.0036 | 1.0036 | 1.0043 |
| 20 | 20_59363112_A_G | 59363112 | 60788056 | rs2426921 | G | A | 0.3715 | 0.0090 | 1.0090 | 1.0067 |
| 20 | 20_61373122_G_A | 61373122 | 62741770 | rs117647014 | A | G | 0.0286 | 0.0046 | 1.0046 | 1.0003 |
| 20 | 20_62692060_A_C | 62692060 | 64060707 | rs6090040 | C | A | 0.5150 | 0.0015 | 1.0015 | 1.0015 |
| 20 | 20_62856686_A_G | 62856686 | 64225333 | rs2320360 | G | A | 0.2700 | 0.0063 | 1.0063 | 1.0034 |
| 20 | 20_62877959_C_T | 62877959 | 64246606 | rs2427628 | T | C | 0.1520 | 0.0106 | 1.0107 | 1.0032 |
| 21 | 21_16227626_A_G | 16227626 | 14855305 | rs7278690 | G | A | 0.9220 | −0.0071 | 0.9929 | 0.9870 |
| 21 | 21_16234888_C_G | 16234888 | 14862567 | rs976051 | G | C | 0.8337 | −0.0008 | 0.9992 | 0.9987 |
| 21 | 21_16353809_A_C | 16353809 | 14981488 | rs17274750 | C | A | 0.0984 | 0.0245 | 1.0248 | 1.0049 |
| 21 | 21_16364756_T_G | 16364756 | 14992435 | rs2822999 | G | T | 0.1732 | 0.0098 | 1.0098 | 1.0034 |
| 21 | 21_16432738_T_C | 16432738 | 15060417 | rs62221393 | C | T | 0.2007 | 0.0106 | 1.0107 | 1.0043 |
| 21 | 21_16455526_T_C | 16455526 | 15083205 | rs2142364 | C | T | 0.8670 | −0.0004 | 0.9996 | 0.9993 |
| 21 | 21_16461783_G_A | 16461783 | 15089462 | rs2823046 | A | G | 0.2714 | −0.0045 | 0.9955 | 0.9976 |
| 21 | 21_16462541_C_G | 16462541 | 15090220 | rs2823049 | G | C | 0.4752 | −0.0003 | 0.9997 | 0.9997 |
| 21 | 21_16481934_G_A | 16481934 | 15109613 | rs34336435 | A | G | 0.1673 | 0.0030 | 1.0030 | 1.0010 |
| 21 | 21_16517891_G_C | 16517891 | 15145570 | rs2049880 | C | G | 0.2190 | 0.0000 | 1.0000 | 1.0000 |
| 21 | 21_16520878_A_G | 16520878 | 15148557 | rs2823094 | G | A | 0.0712 | 0.0091 | 1.0091 | 1.0013 |
| 21 | 21_16520935_A_T | 16520935 | 15148614 | rs2896680 | T | A | 0.2633 | −0.0007 | 0.9993 | 0.9996 |
| 21 | 21_16522082_A_G | 16522082 | 15149761 | rs2823096 | G | A | 0.1860 | −0.0296 | 0.9708 | 0.9892 |
| 21 | 21_16546125_G_T | 16546125 | 15173805 | rs12483016 | T | G | 0.2240 | −0.0012 | 0.9988 | 0.9995 |
| 21 | 21_16547435_A_G | 16547435 | 15175115 | rs11909473 | G | A | 0.2945 | 0.0000 | 1.0000 | 1.0000 |
| 21 | 21_16563309_G_A | 16563309 | 15190989 | rs2823128 | A | G | 0.3244 | −0.0055 | 0.9945 | 0.9964 |
| 21 | 21_16566350_A_G | 16566350 | 15194030 | rs2823130 | G | A | 0.0873 | 0.0117 | 1.0118 | 1.0021 |
| 21 | 21_16574455_C_A | 16574455 | 15202135 | rs2403907 | A | C | 0.3167 | −0.0279 | 0.9725 | 0.9826 |
| 21 | 21_16609549_C_CT | 16609549 | 15237229 | rs34690096 | CT | C | 0.5758 | 0.0000 | 1.0000 | 1.0000 |
| 21 | 21_16645377_C_A | 16645377 | 15273058 | rs35965339 | A | C | 0.0662 | 0.0014 | 1.0014 | 1.0002 |
| 21 | 21_18744081_A_C | 18744081 | 17371762 | rs11908794 | C | A | 0.5505 | 0.0030 | 1.0030 | 1.0033 |
| 21 | 21_18764525_A_G | 18764525 | 17392206 | rs13051956 | G | A | 0.5409 | 0.0031 | 1.0031 | 1.0034 |
| 21 | 21_19797287_C_T | 19797287 | 18424970 | rs4818409 | T | C | 0.4563 | −0.0055 | 0.9945 | 0.9950 |
| 21 | 21_20202110_T_C | 20202110 | 18829792 | rs8130151 | C | T | 0.0544 | 0.0000 | 1.0000 | 1.0000 |
| 21 | 21_20656535_AT_ | 20656535 | 19284218 | 21:20656535_ | A | AT | 0.6869 | 0.0040 | 1.0040 | 1.0055 |
| | A | | | AT_A | | | | | | |
| 21 | 21_20680408_T_G | 20680408 | 19308091 | rs9983076 | G | T | 0.7202 | 0.0044 | 1.0044 | 1.0064 |
| 21 | 21_21275203_A_C | 21275203 | 19902889 | rs62222014 | C | A | 0.0389 | −0.0169 | 0.9832 | 0.9987 |
| 21 | 21_25798887_A_G | 25798887 | 24426573 | rs1119269 | G | A | 0.2401 | 0.0053 | 1.0053 | 1.0026 |
| 21 | 21_25823497_G_T | 25823497 | 24451183 | rs59551976 | T | G | 0.2513 | 0.0023 | 1.0023 | 1.0012 |
| 21 | 21_25892954_G_A | 25892954 | 24520640 | rs62215720 | A | G | 0.2994 | 0.0045 | 1.0045 | 1.0027 |
| 21 | 21_26008883_TTAT | 26008883 | 24636569 | 21:26008883_ | T | TTAT | 0.8764 | −0.0018 | 0.9982 | 0.9969 |
| | ATA_T | | | TTATATA_T | | ATA | | | | |
| 21 | 21_26933938_A_T | 26933938 | 25561626 | rs79507471 | T | A | 0.0337 | −0.0153 | 0.9848 | 0.9990 |
| 21 | 21_27152333_TTC_ | 27152333 | 25780022 | 21:27152333_ | T | TTC | 0.0330 | −0.0002 | 0.9998 | 1.0000 |
| | T | | | TTC_T | | | | | | |
| 21 | 21_28258281_T_G | 28258281 | 26885962 | rs988609 | G | T | 0.0985 | 0.0153 | 1.0154 | 1.0030 |
| 21 | 21_33340086_C_T | 33340086 | 31967774 | rs2833586 | T | C | 0.1009 | −0.0122 | 0.9879 | 0.9976 |
| 21 | 21_33427423_G_A | 33427423 | 32055110 | rs57217265 | A | G | 0.0777 | 0.0000 | 1.0000 | 1.0000 |
| 21 | 21_33428993_G_C | 33428993 | 32056680 | rs11909436 | C | G | 0.1275 | −0.0139 | 0.9862 | 0.9965 |
| 21 | 21_33492560_T_C | 33492560 | 32120248 | rs2833661 | C | T | 0.7813 | 0.0008 | 1.0008 | 1.0013 |
| 21 | 21_33501003_T_C | 33501003 | 32128692 | rs13049602 | C | T | 0.7854 | 0.0082 | 1.0082 | 1.0130 |
| 21 | 21_33876502_T_C | 33876502 | 32504192 | rs7275591 | C | T | 0.1805 | 0.0000 | 1.0000 | 1.0000 |
| 21 | 21_33914960_C_T | 33914960 | 32542650 | rs2833872 | T | C | 0.2097 | 0.0149 | 1.0150 | 1.0063 |
| 21 | 21_33941082_G_C | 33941082 | 32568772 | rs844989 | C | G | 0.2326 | 0.0012 | 1.0012 | 1.0006 |
| 21 | 21_34251327_C_T | 34251327 | 32879019 | rs2833983 | T | C | 0.4091 | 0.0036 | 1.0036 | 1.0030 |
| 21 | 21_35815455_A_C | 35815455 | 34443157 | rs58060663 | C | A | 0.0373 | 0.0085 | 1.0085 | 1.0006 |
| 21 | 21_36080290_C_T | 36080290 | 34707992 | rs6517254 | T | C | 0.3724 | −0.0026 | 0.9974 | 0.9981 |
| 21 | 21_36253194_T_A | 36253194 | 34880897 | rs2298354 | A | T | 0.2505 | 0.0037 | 1.0037 | 1.0019 |
| 21 | 21_38622181_A_G | 38622181 | 37249880 | rs147700715 | G | A | 0.0178 | 0.0130 | 1.0131 | 1.0005 |
| 21 | 21_38642243_C_T | 38642243 | 37269942 | rs117110236 | T | C | 0.0214 | 0.0197 | 1.0199 | 1.0009 |
| 21 | 21_38784359_A_C | 38784359 | 37412057 | rs13052173 | C | A | 0.0666 | −0.0144 | 0.9857 | 0.9981 |
| 21 | 21_40709960_C_T | 40709960 | 39338034 | rs1963676 | T | C | 0.5774 | −0.0046 | 0.9954 | 0.9947 |
| 21 | 21_42191862_A_G | 42191862 | 40819936 | rs2837838 | G | A | 0.9293 | −0.0158 | 0.9843 | 0.9711 |
| 21 | 21_44472345_G_A | 44472345 | 43052235 | rs57063354 | A | G | 0.0376 | 0.0124 | 1.0125 | 1.0009 |
| 21 | 21_44475877_C_T | 44475877 | 43055767 | rs8132811 | T | C | 0.2049 | 0.0025 | 1.0025 | 1.0010 |
| 21 | 21_44477543_G_C | 44477543 | 43057433 | rs234702 | C | G | 0.9190 | −0.0110 | 0.9891 | 0.9800 |
| 21 | 21_45467448_C_T | 45467448 | 44047567 | rs9974857 | T | C | 0.0550 | −0.0197 | 0.9805 | 0.9979 |
| 21 | 21_45740823_A_G | 45740823 | 44320940 | rs3827256 | G | A | 0.6519 | −0.0028 | 0.9972 | 0.9964 |
| 21 | 21_47718961_CT_C | 47718961 | 46299047 | 21:47718961_ | C | CT | 0.8400 | −0.0022 | 0.9978 | 0.9963 |
| | | | | CT_C | | | | | | |
| 21 | 21_47762932_G_A | 47762932 | 46343018 | rs4818836 | A | G | 0.0355 | 0.0314 | 1.0319 | 1.0023 |
| 21 | 21_47982363_G_A | 47982363 | 46562450 | rs2070430 | A | G | 0.4970 | 0.0057 | 1.0057 | 1.0057 |
| 22 | 22_19151419_G_A | 19151419 | 19163906 | rs66493436 | A | G | 0.2310 | −0.0061 | 0.9939 | 0.9972 |
| 22 | 22_19619489_CCT_ | 19619489 | 19631966 | 22:19619489_ | C | CCT | 0.0322 | 0.0122 | 1.0123 | 1.0008 |
| | C | | | CCT_C | | | | | | |
| 22 | 22_19765322_C_T | 19765322 | 19777799 | rs4819521 | T | C | 0.4444 | −0.0076 | 0.9924 | 0.9933 |
| 22 | 22_19766107_G_A | 19766107 | 19778584 | rs41297994 | A | G | 0.1812 | −0.0023 | 0.9977 | 0.9992 |
| 22 | 22_19766137_C_T | 19766137 | 19778614 | rs9798754 | T | C | 0.3798 | −0.0035 | 0.9965 | 0.9973 |
| 22 | 22_28341053_C_T | 28341053 | 27945065 | rs9985181 | T | C | 0.1698 | 0.0064 | 1.0064 | 1.0022 |
| 22 | 22_29077631_C_C | 29077631 | 28681643 | rs34661406 | CA | C | 0.0230 | 0.0000 | 1.0000 | 1.0000 |
| | A | | | | | | | | | |
| 22 | 22_29121087_A_G | 29121087 | 28725099 | rs17879961 | G | A | 0.0054 | 0.0861 | 1.0899 | 1.0010 |
| 22 | 22_29135543_G_A | 29135543 | 28739555 | rs5997390 | A | G | 0.0870 | 0.0255 | 1.0258 | 1.0045 |
| 22 | 22_29157487_G_A | 29157487 | 28761499 | rs6005864 | A | G | 0.4206 | 0.0044 | 1.0044 | 1.0037 |
| 22 | 22_29167536_CT_C | 29167536 | 28771548 | rs773867741 | C | CT | 0.2511 | 0.0001 | 1.0001 | 1.0001 |
| 22 | 22_29203724_C_T | 29203724 | 28807736 | rs34134147 | T | C | 0.0209 | 0.0539 | 1.0554 | 1.0023 |
| 22 | 22_29272637_C_T | 29272637 | 28876649 | rs62235753 | T | C | 0.0177 | 0.0455 | 1.0466 | 1.0017 |
| 22 | 22_29305061_G_T | 29305061 | 28909073 | rs7364229 | T | G | 0.2022 | 0.0028 | 1.0028 | 1.0011 |
| 22 | 22_29375026_C_T | 29375026 | 28979038 | rs10854810 | T | C | 0.3024 | −0.0020 | 0.9980 | 0.9988 |
| 22 | 22_29406219_A_T | 29406219 | 29010231 | rs13054095 | T | A | 0.0224 | 0.0000 | 1.0000 | 1.0000 |
| 22 | 22_29529148_A_G | 29529148 | 29133160 | rs5997447 | G | A | 0.0470 | 0.0000 | 1.0000 | 1.0000 |
| 22 | 22_29551872_A_G | 29551872 | 29155884 | rs132289 | G | A | 0.9846 | −0.1118 | 0.8942 | 0.8026 |
| 22 | 22_29669648_G_C | 29669648 | 29273659 | rs4820803 | C | G | 0.1712 | −0.0014 | 0.9986 | 0.9995 |
| 22 | 22_30160747_C_T | 30160747 | 29764758 | rs8142292 | T | C | 0.0400 | −0.0208 | 0.9794 | 0.9984 |
| 22 | 22_30161801_A_G | 30161801 | 29765812 | rs5997518 | G | A | 0.0554 | 0.0014 | 1.0014 | 1.0002 |
| 22 | 22_30868599_G_A | 30868599 | 30472612 | rs11705494 | A | G | 0.0824 | −0.0033 | 0.9967 | 0.9995 |
| 22 | 22_32732644_A_G | 32732644 | 32336657 | rs61693297 | G | A | 0.1994 | −0.0142 | 0.9859 | 0.9944 |
| 22 | 22_32771017_G_T | 32771017 | 32375030 | rs5754049 | T | G | 0.2577 | −0.0048 | 0.9952 | 0.9975 |
| 22 | 22_33168255_T_C | 33168255 | 32772269 | rs76307741 | C | T | 0.0352 | −0.0159 | 0.9842 | 0.9989 |
| 22 | 22_34514287_T_C | 34514287 | 34118298 | rs9609951 | C | T | 0.0363 | −0.0312 | 0.9693 | 0.9978 |
| 22 | 22_37369880_G_A | 37369880 | 36973839 | rs2251446 | A | G | 0.4241 | 0.0081 | 1.0081 | 1.0069 |
| 22 | 22_37750349_T_C | 37750349 | 37354309 | rs2899289 | C | T | 0.6411 | −0.0069 | 0.9931 | 0.9912 |
| 22 | 22_37751638_CA_ | 37751638 | 37355598 | 22:3775163_8 | C | CA | 0.4035 | −0.0005 | 0.9995 | 0.9996 |
| | C | | | CA_C | | | | | | |
| 22 | 22_37770357_A_G | 37770357 | 37374317 | rs7292751 | G | A | 0.3783 | 0.0017 | 1.0017 | 1.0013 |
| 22 | 22_37771158_G_A | 37771158 | 37375118 | rs2071857 | A | G | 0.4266 | 0.0026 | 1.0026 | 1.0022 |
| 22 | 22_38195596_G_A | 38195596 | 37799589 | rs8140423 | A | G | 0.2200 | −0.0024 | 0.9976 | 0.9989 |
| 22 | 22_38200124_C_T | 38200124 | 37804117 | rs11703407 | T | C | 0.3184 | 0.0000 | 1.0000 | 1.0000 |
| 22 | 22_38426471_G_G | 38426471 | 38030464 | rs5845363 | GC | G | 0.1820 | 0.0020 | 1.0020 | 1.0007 |
| | C | | | | | | | | | |
| 22 | 22_38568833_C_G | 38568833 | 38172826 | rs738321 | G | C | 0.3805 | −0.0125 | 0.9876 | 0.9906 |
| 22 | 22_38583315_AAA | 38583315 | 38187308 | | AAAAGAA | AAAA | 0.2805 | −0.0071 | 0.9929 | 0.9960 |
| | AG_AAAAGAAAG | | | | AG | G | | | | |
| 22 | 22_38605067_T_G | 38605067 | 38209060 | rs11705176 | G | T | 0.3078 | 0.0000 | 1.0000 | 1.0000 |
| 22 | 22_38613851_AAA | 38613851 | 38217844 | 22:38613851_ | A | AAAA | 0.3425 | 0.0000 | 1.0000 | 1.0000 |
| | AG_A | | | AAAAG_A | | G | | | | |
| 22 | 22_38757568_AAA | 38757568 | 38361563 | 22:38757568_ | A | AAAA | 0.3887 | 0.0044 | 1.0044 | 1.0034 |
| | ATAAATAAATAAAT | | | AAAATAAATA | | TAAA | | | | |
| | _A | | | AATAAAT_A | | TAAA | | | | |
| | | | | | | TAAA | | | | |
| | | | | | | T | | | | |
| 22 | 22_39194068_C_T | 39194068 | 38798063 | rs9619765 | T | C | 0.0348 | −0.0284 | 0.9720 | 0.9981 |
| 22 | 22_39286208_A_T | 39286208 | 38890203 | rs132502 | T | A | 0.3899 | −0.0007 | 0.9993 | 0.9995 |
| 22 | 22_39343916_T_A | 39343916 | 38947911 | rs5750715 | A | T | 0.2541 | 0.0076 | 1.0076 | 1.0039 |
| 22 | 22_39524131_G_A | 39524131 | 39128126 | rs139376 | A | G | 0.5276 | 0.0033 | 1.0033 | 1.0035 |
| 22 | 22_39626375_A_C | 39626375 | 39230370 | rs4821874 | C | A | 0.7286 | −0.0047 | 0.9953 | 0.9932 |
| 22 | 22_40121441_C_T | 40121441 | 39725436 | rs10427962 | T | C | 0.0821 | −0.0047 | 0.9953 | 0.9992 |
| 22 | 22_40242010_C_A | 40242010 | 39846005 | rs78943183 | A | C | 0.0319 | −0.0158 | 0.9843 | 0.9990 |
| 22 | 22_40476546_A_G | 40476546 | 40080542 | rs118176772 | G | A | 0.0261 | 0.0267 | 1.0271 | 1.0014 |
| 22 | 22_40496066_TTTT | 40496066 | 40100062 | 22:40496066_ | T | TTTT | 0.2343 | 0.0000 | 1.0000 | 1.0000 |
| | TATTA_T | | | TTTTTATTA_T | | TATT | | | | |
| | | | | | | A | | | | |
| 22 | 22_40810946_T_C | 40810946 | 40414942 | rs729982 | C | T | 0.1310 | 0.0109 | 1.0110 | 1.0029 |
| 22 | 22_40904707_CT_C | 40904707 | 40508703 | 22:40904707_ | C | CT | 0.1099 | 0.0624 | 1.0644 | 1.0142 |
| | | | | CT_C | | | | | | |
| 22 | 22_40930212_C_T | 40930212 | 40534208 | rs2413624 | T | C | 0.6352 | −0.0088 | 0.9912 | 0.9889 |
| 22 | 22_41049402_G_T | 41049402 | 40653398 | rs56051217 | T | G | 0.1000 | 0.0020 | 1.0020 | 1.0004 |
| 22 | 22_41059547_C_C | 41059547 | 40663543 | rs562341638 | CA | C | 0.1951 | 0.0064 | 1.0064 | 1.0025 |
| | A | | | | | | | | | |
| 22 | 22_41062415_CA_ | 41062415 | 40666411 | 22:41062415_ | C | CA | 0.2277 | 0.0000 | 1.0000 | 1.0000 |
| | C | | | CA_C | | | | | | |
| 22 | 22_43110115_A_G | 43110115 | 42714109 | rs5758891 | G | A | 0.5772 | 0.0022 | 1.0022 | 1.0025 |
| 22 | 22_43178526_A_C | 43178526 | 42782520 | rs552455424 | C | A | 0.0264 | 0.0106 | 1.0107 | 1.0006 |
| 22 | 22_43433100_C_T | 43433100 | 43037094 | rs9611990 | T | C | 0.1144 | −0.0176 | 0.9826 | 0.9960 |
| 22 | 22_43468665_G_T | 43468665 | 43072659 | rs28599928 | T | G | 0.1097 | −0.0073 | 0.9927 | 0.9984 |
| 22 | 22_43560309_AAC | 43560309 | 43164303 | 22:43560309_ | A | AACA | 0.4483 | −0.0024 | 0.9976 | 0.9979 |
| | ACAC_A | | | AACACAC_A | | CAC | | | | |
| 22 | 22_44598088_A_G | 44598088 | 44202208 | rs139164 | G | A | 0.9292 | −0.0057 | 0.9943 | 0.9895 |
| 22 | 22_45276346_C_T | 45276346 | 44880466 | rs17542188 | T | C | 0.4064 | −0.0048 | 0.9952 | 0.9961 |
| 22 | 22_45295419_G_C | 45295419 | 44899539 | rs4823408 | C | G | 0.4000 | 0.0023 | 1.0023 | 1.0018 |
| 22 | 22_45301457_C_T | 45301457 | 44905577 | rs5766175 | T | C | 0.3688 | 0.0002 | 1.0002 | 1.0001 |
| 22 | 22_45321906_C_CC | 45321906 | 44926026 | 22:45321906_ | CCTGTGG | C | 0.3696 | 0.0000 | 1.0000 | 1.0000 |
| | TGTGGTGGGAAGG | | | C_CCTGTGGTG | TGGGAAG | | | | | |
| | CCCACACAGCACGG | | | GGAAGGCCCA | GCCCACA | | | | | |
| | CCCTGTGGTGGATT | | | CACAGCACGG | CAGCACG | | | | | |
| | CCCAGGGAAGTGG | | | CCCTGTGGTG | GCCCTGT | | | | | |
| | GTT | | | GATTCCCAGG | GGTGGAT | | | | | |
| | | | | GAAGTGGGTT | TCCCAGG | | | | | |
| | | | | | GAAGTGG | | | | | |
| | | | | | GTT | | | | | |
| 22 | 22_46156825_G_C | 46156825 | 45760945 | rs4823322 | C | G | 0.4169 | −0.0040 | 0.9960 | 0.9967 |
| 22 | 22_46160624_G_A | 46160624 | 45764744 | rs138203 | A | G | 0.7477 | −0.0047 | 0.9953 | 0.9930 |
| 22 | 22_46175966_CT_C | 46175966 | 45780086 | 22:46175966_ | C | CT | 0.8215 | −0.0023 | 0.9977 | 0.9962 |
| | | | | CT_C | | | | | | |
| 22 | 22_46274072_A_G | 46274072 | 45878192 | rs77848624 | G | A | 0.1622 | 0.0064 | 1.0064 | 1.0021 |
| 22 | 22_46283297_G_A | 46283297 | 45887417 | rs28512361 | A | G | 0.1117 | 0.0310 | 1.0315 | 1.0070 |
| 22 | 22_46320819_T_C | 46320819 | 45924939 | rs28548232 | C | T | 0.1307 | 0.0032 | 1.0032 | 1.0008 |
| 22 | 22_49094148_T_C | 49094148 | 48698336 | rs56307769 | C | T | 0.5791 | −0.0042 | 0.9958 | 0.9952 |
| 22 | 22_49096694_C_T | 49096694 | 48700882 | rs130129 | T | C | 0.3437 | −0.0012 | 0.9988 | 0.9992 |
| 22 | 22_49312374_A_C | 49312374 | 48916562 | rs13055787 | C | A | 0.9377 | −0.0083 | 0.9917 | 0.9846 |
| 22 | 22_50602304_T_A | 50602304 | 50163875 | rs200311816 | A | T | 0.0299 | 0.0050 | 1.0050 | 1.0003 |

### Genetic Risk Score

An individual's “polygenic risk score” can be defined as the weighted sum of the individuals' genotypes at multiple genetic loci. In other words, they are the linear combinations of the risk alleles across a set of candidate SNPs. For example, a PRS for individual i can be calculated as:

$${PRS}_{i} = {β}_{1} {x}_{i 1} + {β}_{2} {x}_{i 2} + … + {β}_{j} {x}_{ij} + … + {β}_{p} {x}_{ip} , \qquad ( 1 )$$

where xᵢⱼ∈{0,1,2} are risk allele counts and βj are the weights for SNP j=1; : : : ; p.

The PRS can also be represented as: PRS=Σ (No of Risk Alleles×β coefficient).

In one embodiment, the key steps to construct a polygenic risk score (PRS) are to determine which SNPs to include and how to weight their effects. In one embodiment, the maxCT and SCT methods (Privé et al., 2019) are used. These methods are based on clumping and thresholding. The aim of clumping and thresholding is to remove correlated SNPs while keeping the most important SNPs in the PRS. In order to do this, the values of a range of hyperparameters including the correlation threshold (r2), the clumping window size (kb) and the p-value significance threshold (p) are decided. Different selection of these hyperparameters values would in general give a different section of SNPs to include. For the weights, the reported GWAS coefficients (i.e., regression coefficients or log odds ratios) from external published GWAS can be used. The core idea of the maxCT and SCT procedures is to select a set of different hyperparameters values and compute a PRS for each combination of these values.

This will usually create a large number of PRSs, for example, around 100,000 vectors of PRSs for a typical GWAS. After constructing these RPSs, there are two approaches to create the final PRS. In maxCT, the PRS that has the strongest predictive performance (e.g., largest AUC) are selected as the final PRS. In SCT, the PRSs by a penalized logistic regression model, for example, the popular lasso procedure are combined. Since the outcome of SCT will be a linear combination of PRSs, where each PRS is again a linear combination of variants, the final PRS still has the form of (1), which means the effect sizes of SNPs can be obtained and used for prediction.

In an preferred example, a log-additive risk model can then be used to define three genotypes AA, AB, and BB for a single SNP having relative risk values of 1, OR, and OR², under a rare disease model, where OR is the previously reported disease odds ratio for the high-risk allele, B, vs the low-risk allele, A. If the B allele has frequency (p), then these genotypes have population frequencies of (1−p)², 2p(1−p), and p², assuming Hardy-Weinberg equilibrium. The genotype relative risk values for each SNP can then be scaled so that based on these frequencies the average relative risk in the population is 1. Specifically, given the unscaled population average relative risk:

$$( μ ) = {( 1 - p )}^{2} + 2 p ( 1 - p ) OR + {p}^{2} O {R}^{2}$$

Adjusted risk values 1/μ, OR/μ, and OR²/μ are used for AA, AB, and BB genotypes. Missing genotypes are assigned a relative risk of 1. The following formula can be used to define the genetic risk:
SNP₁×SNP₂×SNP₃×SNP₄×SNP₅×SNP₆×SNP₇,×SNP₈, etc.

Similar calculations can be performed for non-SNP polymorphisms.

An alternate method for calculating the composite SNP risk is described in Mavaddat et al. (2015). In this example, the following formula is used;

$$PRS = {β}_{1} {x}_{1} + {β}_{2} {x}_{2} + … + {β}_{κ} {x}_{κ} + {β}_{n} {x}_{n}$$

where β_{κ} is the per-allele log odds ratio (OR) for breast cancer associated with the minor allele for SNP K, and xx the number of alleles for the same SNP (0, 1 or 2), n is the total number of SNPs and PRS is the polygenic risk score (which can also be referred to as composite SNP risk).

It is envisaged that the “risk” of a human subject for developing breast cancer can be provided as a relative risk (or risk ratio) or an absolute risk as required.

In an embodiment, the genetic risk assessment obtains the “relative risk” of a human subject for developing breast cancer. Relative risk (or risk ratio), measured as the incidence of a disease in individuals with a particular characteristic (or exposure) divided by the incidence of the disease in individuals without the characteristic, indicates whether that particular exposure increases or decreases risk. Relative risk is helpful to identify characteristics that are associated with a disease, but by itself is not particularly helpful in guiding screening decisions because the frequency of the risk (incidence) is cancelled out.

In another embodiment, the genetic risk assessment obtains the “absolute risk” of a human subject for developing breast cancer. Absolute risk is the numerical probability of a human subject developing breast cancer within a specified period (e.g. 5, 10, 15, 20 or more years). It reflects a human subject's risk of developing breast cancer insofar as it does not consider various risk factors in isolation.

### Breast Cancer Risk Assessment

In an embodiment, when using the % breast density as the mammographic density score a risk (Breast Cancer Risk (BRISK)) score is determined using;

$$brisk 1 \_N = \frac{( cumul 1 \_b \_N - cumul 1 \_b )}{( 1 - cumul 1 \_b )}$$

where:
N is the number of years of risk to calculate,
incid_b is the ethnicity specific cumulative breast cancer incidence up to current age, and
incid_bN is the ethnicity specific cumulative breast cancer incidence up to current age plus N years.

In another embodiment, % breast density as the mammographic density score is used to estimate lifetime risk (Breast Cancer Risk (BRISK)) score using;

$$brisk 1 \_rem \_life = \frac{( cumul 1 \_full \_life - cumul 1 \_b )}{( 1 - cumul 1 \_b )}$$

where:
incid_b is the ethnicity specific cumulative breast cancer incidence up to current age, and
incid_full_life is the ethnicity specific cumulative breast cancer incidence up to 90 years (i.e. full-lifetime risk).

In an embodiment,

$$cumul 1 \_b = 1 - {e}^{- risk\_mod 1 \times incid \_ b} \\ cumul 1 \_ b \_ N = 1 - {e}^{- risk \_ mod 1 \times incid \_ b \_ N} \\ cumul 1 \_full \_life = 1 - {e}^{- risk \_ mod 1 \times incid \_ full \_ life}$$

Furthermore,

$$Inrisk 1 = ln ( PRS ) \times {β}_{PRS} + ( sqrt ( percent\_density ) - sqrt ( {mean}_{DENS} ) ) \times {β}_{DENS} + ln ( {fh\_risk}_{-} 1 ) + ln ( {fh\_risk}_{-} 2 ) + {intercept}_{PRE} + ( ( ln ( bmi / 5 ) - ln ( {mean}_{BMI} / 5 ) ) \times {β}_{PRE} ) if pre ‐ menopausal + {intercept}_{POST} + ( ( ln ( bmi / 5 ) - ln ( {mean}_{BMI} / 5 ) ) \times {β}_{POST} ) if post ‐ menopausal$$

then:

risk_mod1=e^{lnrisk1}

where:
β_{PRS} is the natural logarithm of the relative risk associated with a one-unit increase in the PRS (such as the β_{PRS} is between 0.93 and 1.13, or between 0.98 and 1.08, or is 1.03);
mean_{DENS}=the mean percent density in the population in which the test is used (such as the mean_{DENS} is between 15.14 and 19.14, or between 16.14 and 18.14, or is 17.14);
β_{DENS} is the natural logarithm of the relative risk associated with a one-unit increase in the square root of percent density (such as the β_{DENS} is between 0.02 and 0.22, or between 0.07 and 0.17 or is 0.12);
intercept_{PRE} is the equation intercept for pre-menopausal women (such as the intercept_{PRE} is between −0.01 and −0.07, or between −0.03 and −0.05 or is −0.04);
intercept_{POST} is the equation intercept for post-menopausal women (such as the intercept_{POST} is between 0.02 and 0.07, or between 0.04 and 0.06, or is 0.05);
mean_{BMI} is the mean body mass index (kg/m²) in the population in which the test is used (such as the mean_{BMI} is between 23.27 and 27.27, or between 24.27 and 26.27, or is 25.27);
β_{PRE} is the natural logarithm of the relative risk associated with being pre-menopausal (such as the β_{PRE} is between −0.15 and −0.55, or between −0.25 and −0.45 or is −0.35); and
β_{POST} is the natural logarithm of the relative risk associated with being post-menopausal (such as the β_{POST} is between 0.68 and 1.08, or between 0.78 and −098 or is 0.88).

In an embodiment, β_{PRS}=1.03, mean_{DENS}=17.14, β_{DENS}=0.12, intercept_{PRE}=−0.04, intercept_{POST}=0.05, mean_{BMI}=25.27, β_{PRE}=−0.35 and β_{POST}=0.88.

In an embodiment, when using the BI-RADS category as the mammographic density score a risk (Breast Cancer Risk (BRISK)) score is determined using;

### N-Year Risk

$$brisk 2 \_N = \begin{matrix}( cumul 2 \_b \_N - cumul 2 \_b ) \\ ( 1 - cumul 2 \_b )\end{matrix}$$

where:
N is the number of years of risk to calculate,
incid_b is the ethnicity specific cumulative breast cancer incidence up to current age, and
incid_bN is the ethnicity specific cumulative breast cancer incidence up to current age plus N years.

In another embodiment, BI-RADS category as the mammographic density score is used to estimate a lifetime risk (Breast Cancer Risk (BRISK)) score using;

$$brisk 2 \_rem \_life = \frac{( cumul 2 \_full \_life - cumul 2 \_b )}{( 1 - cumul 2 \_b )}$$

where:
incid_b is the ethnicity specific cumulative breast cancer incidence up to current age, and
incid_full_life is the ethnicity specific cumulative breast cancer incidence up to 90 years (i.e. full-lifetime risk).

In an embodiment,

$$cumul 2 \_b = 1 - {e}^{- risk\_mod 2 \times incid \_ b} \\ cumul 2 \_ b \_ N = 1 - {e}^{- risk \_ mod 2 \times incid \_ b \_ N} \\ cumul 2 \_full \_life = 1 - {e}^{- risk \_ mod 2 \times incid \_ full \_ life}$$

Furthermore,

$$lnrisk 2 = ln ( PRS ) \times {β}_{PRS} + X if BI - RADS category is A or B + Y if BI - RADS category is C or D + ln ( fh\_risk \_ 1 ) + ln ( fh\_risk \_ 2 ) + {intercept}_{PRE} + ( ( ln ( bmi / 5 ) - ln ( {mean}_{BMI} / 5 ) ) \times {β}_{PRE} ) if pre - menopausal + {intercept}_{POST} + ( ( ln ( bmi / 5 ) - ln ( {mean}_{BMI} / 5 ) ) \times {β}_{POST} ) if post - menopausal$$

then:

risk_mod2=e^{lnrisk2}

where:
X is between −0.05 and −0.25, or between −0.1 and −0.2 or is −0.15;
Y is between 0.14 and 0.34, or between 0.19 and 0.29, or is 0.24;
β_{PRS} is the natural logarithm of the relative risk associated with a one-unit increase in the PRS (such as the β_{PRS} is between 0.93 and 1.13, or between 0.98 and 1.08, or is 1.03);
intercept_{PRE} is the equation intercept for pre-menopausal women (such as the intercept_{PRE} is between −0.01 and −0.07, or between −0.03 and −0.05 or is −0.04);
intercept_{POST} is the equation intercept for post-menopausal women (such as the intercept_{POST} is between 0.02 and 0.07, or between 0.04 and 0.06, or is 0.05);
mean_{BMI} is the mean body mass index (kg/m²) in the population in which the test is used (such as the mean_{BMI} is between 23.27 and 27.27, or between 24.27 and 26.27, or is 25.27);
β_{PRE} is the natural logarithm of the relative risk associated with being pre-menopausal (such as the β_{PRE} is between −0.15 and −0.55, or between −0.25 and −0.45 or is −0.35); and
β_{POST} is the natural logarithm of the relative risk associated with being post-menopausal (such as the β_{POST} is between 0.68 and 1.08, or between 0.78 and −098 or is 0.88).

In an embodiment, X=−0.15, Y=0.24, β_{PRS}=1.03, intercept_{PRE}=−0.04, intercept_{POST}=0.05, mean_{BMI}=25.27, β_{PRE}=−0.35 and β_{POST}=0.88.

In an embodiment, one or more threshold value(s) are set for determining a particular action such as the need for routine diagnostic testing or preventative therapy. For example, a score determined using a method of the invention is compared to a pre-determined threshold, and if the score is higher than the threshold a recommendation is made to take the pre-determined action. Methods of setting such thresholds have now become widely used in the art and are described in, for example, US20140018258. For example, at present The American Society of Clinical Oncology recommends that Tamoxifen be offered for the chemoprevention of breast cancer for women who have a 5-year risk exceeding >1.66 or ≥1.67, the United States Preventive Services Task Force recommends that Tamoxifen be offered for the chemoprevention of breast cancer for women who have a 5-year risk exceeding 3.00%, and The National Comprehensive Cancer Network recommends that increased screening, including the option of annual MRIs be offered to women who have a lifetime risk of breast cancer exceeding 20.0%.

### Treatment

After performing the methods of the present disclosure treatment may be prescribed or administered to the subject.

Accordingly, in an embodiment, the methods of the present disclosure relate to an anti-cancer therapy for use in preventing or reducing the risk of breast cancer in a human subject at risk thereof.

One of skill in the art will appreciate that breast cancer is a heterogeneous disease with distinct clinical outcomes (Sorlie et al., 2001). For example, it is discussed in the art that breast cancer may be oestrogen receptor positive or oestrogen receptor negative. In one embodiment, it is not envisaged that the methods of the present disclosure be limited to assessing the risk of developing a particular type or subtype of breast cancer. For example, it is envisaged that the methods of the present disclosure can be used to assess the risk of developing oestrogen receptor positive or oestrogen receptor negative breast cancer. In another embodiment, the methods of the present disclosure are used to assess the risk of developing oestrogen receptor positive breast cancer. In another embodiment, the methods of the present disclosure are used to assess the risk of developing oestrogen receptor negative breast cancer. In another embodiment, the methods of the present disclosure are used to assess the risk of developing metastatic breast cancer. In an example, a therapy that inhibits oestrogen is prescribed or administered to the subject.

In another example, a chemopreventative is prescribed or administered to the subject. There are two main classes of drugs currently utilized for breast cancer chemoprevention:
(1) Selective Estrogen Receptor Modulators (SERMs) which block oestrogen molecules from binding to their associated cellular receptor. This class of drugs includes for example Tamoxifen and Raloxifene.
(2) Aromatase Inhibitors which inhibit the conversion of androgens into oestrogens by the aromatase enzyme Ie reducing the production of oestrogens. This class of drugs includes for example Exemestane, Letrozole, Anastrozole, Vorozole, Formestane, Fadrozole.

In an example, a SERM or an aromatase inhibitor is prescribed or administered to the subject.

In an example, Tamoxifen, Raloxifene, Exemestane, Letrozole, Anastrozole, Vorozole, Formestane or Fadrozole is prescribed or administered to a subject.

In an embodiment, the methods of the present disclosure are used to assess the risk of a human female subject for developing breast cancer and administering a treatment appropriate for the risk of developing breast cancer. For example, when performing the methods of the present disclosure indicates a high risk of breast cancer an aggressive chemopreventative treatment regimen can be established. In contrast, when performing the methods of the present disclosure indicates a moderate risk of breast cancer a less aggressive chemopreventative treatment regimen can be established. Alternatively, when performing the methods of the present disclosure indicates a low risk of breast cancer a chemopreventative treatment regimen need not be established. It is envisaged that the methods of the present disclosure can be performed over time so that the treatment regimen can be modified in accordance with the subject's risk of developing breast cancer.

### Marker Detection Strategies

Amplification primers for amplifying markers (e.g., marker loci) and suitable probes to detect such markers or to genotype a sample with respect to multiple marker alleles, can be used in the disclosure. For example, primer selection for long-range PCR is described in U.S. Ser. No. 10/042,406 and U.S. Ser. No. 10/236,480; for short-range PCR, U.S. Ser. No. 10/341,832 provides guidance with respect to primer selection. Also, there are publicly available programs such as “Oligo” available for primer design. With such available primer selection and design software, the publicly available human genome sequence and the polymorphism locations, one of skill can construct primers to amplify the polymorphisms to practice the disclosure. Further, it will be appreciated that the precise probe to be used for detection of a nucleic acid comprising a polymorphism (e.g., an amplicon comprising the polymorphism) can vary, e.g., any probe that can identify the region of a marker amplicon to be detected can be used in conjunction with the present disclosure. Further, the configuration of the detection probes can, of course, vary. Thus, the disclosure is not limited to the sequences recited herein.

Indeed, it will be appreciated that amplification is not a requirement for marker detection, for example one can directly detect unamplified genomic DNA simply by performing a Southern blot on a sample of genomic DNA.

Typically, molecular markers are detected by any established method available in the art, including, without limitation, allele specific hybridization (ASH), detection of extension, array hybridization (optionally including ASH), or other methods for detecting polymorphisms, amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, and single-strand conformation polymorphisms (SSCP) detection.

As the skilled person will appreciate, the sequence of the genomic region to which these oligonucleotides hybridize can be used to design primers which are longer at the 5′ and/or 3′ end, possibly shorter at the 5′ and/or 3′ (as long as the truncated version can still be used for amplification), which have one or a few nucleotide differences (but nonetheless can still be used for amplification), or which share no sequence similarity with those provided but which are designed based on genomic sequences close to where the specifically provided oligonucleotides hybridize and which can still be used for amplification.

In some embodiments, the primers are radiolabelled, or labelled by any suitable means (e.g., using a non-radioactive fluorescent tag), to allow for rapid visualization of differently sized amplicons following an amplification reaction without any additional labelling step or visualization step. In some embodiments, the primers are not labelled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers be limited to generating an amplicon of any particular size. For example, the primers used to amplify the marker loci and alleles herein are not limited to amplifying the entire region of the relevant locus, or any subregion thereof. The primers can generate an amplicon of any suitable length for detection. In some embodiments, marker amplification produces an amplicon at least nucleotides in length, or alternatively, at least 50 nucleotides in length, or alternatively, at least 100 nucleotides in length, or alternatively, at least 200 nucleotides in length. Amplicons of any size can be detected using the various technologies described herein. Differences in base composition or size can be detected by conventional methods such as electrophoresis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook et al. (supra).

PCR detection using dual-labelled fluorogenic oligonucleotide probes, commonly referred to as “TaqMan™” probes, can also be performed according to the present disclosure. These probes are composed of short (e.g., 20-25 base) oligodeoxynucleotides that are labelled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO 92/02638.

Other similar methods include e.g. fluorescence resonance energy transfer between two adjacently hybridized probes, e.g., using the “LightCycler®” format described in U.S. Pat. No. 6,174,670.

Array-based detection can be performed using commercially available arrays, e.g., from Affymetrix (Santa Clara, Calif.) or other manufacturers. Reviews regarding the operation of nucleic acid arrays include Sapolsky et al. (1999); Lockhart (1998); Fodor (1997a); Fodor (1997b) and Chee et al. (1996). Array based detection is one preferred method for identification markers of the disclosure in samples, due to the inherently high-throughput nature of array based detection.

The nucleic acid sample to be analysed is isolated, amplified and, typically, labelled with biotin and/or a fluorescent reporter group. The labelled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labelled nucleic acid, which is now bound to the probe array. Probes that most clearly match the labelled nucleic acid produce stronger signals than those that have mismatches. Since the sequence and position of each probe on the array are known, by complementarity, the identity of the nucleic acid sample applied to the probe array can be identified.

Markers and polymorphisms can also be detected using DNA sequencing. DNA sequencing methods are well known in the art and can be found for example in Ausubel et al, eds., Short Protocols in Molecular Biology, 3rd ed., Wiley, (1995) and Sambrook et al, Molecular Cloning, 2nd ed., Chap. 13, Cold Spring Harbor Laboratory Press, (1989). Sequencing can be carried out by any suitable method, for example, dideoxy sequencing, chemical sequencing, or variations thereof.

Suitable sequencing methods also include Second Generation, Third Generation, or Fourth Generation sequencing technologies, all referred to herein as “next generation sequencing”, including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. A review of some such technologies can be found in (Morozova and Marra, 2008), herein incorporated by reference. Accordingly, in some embodiments, performing a genetic risk assessment as described herein involves detecting the at least two polymorphisms by DNA sequencing. In an embodiment, the at least two polymorphisms are detected by next generation sequencing.

Next generation sequencing (NGS) methods share the common feature of massively parallel, high-throughput strategies, with the goal of lower costs in comparison to older sequencing methods (see, Voelkerding et al., 2009; MacLean et al., 2009).

A number of such DNA sequencing techniques are known in the art, including fluorescence-based sequencing methodologies (Birren et al., 1997). In some embodiments, automated sequencing techniques are used. In some embodiments, parallel sequencing of partitioned amplicons is used (WO2006084132). In some embodiments, DNA sequencing is achieved by parallel oligonucleotide extension (See, e.g., U.S. Pat. Nos. 5,750,341 and 6,306,597). Additional examples of sequencing techniques include the Church polony technology (Mitra et al., 2003; Shendure et al., 2005; U.S. Pat. Nos. 6,432,36; 6,485,944; 6,511,803), the 454 picotiter pyrosequencing technology (Margulies et al., 2005; US20050130173), the Solexa single base addition technology (Bennett et al., 2005; U.S. Pat. Nos. 6,787,308; 6,833,246), the Lynx massively parallel signature sequencing technology (Brenner et al., 2000; U.S. Pat. Nos. 5,695,934; 5,714,330), and the Adessi PCR colony technology (Adessi et al., 2000).

### Computer Implemented Method

It is envisaged that the methods of the present disclosure may be implemented by a system such as a computer implemented method. For example, the system may be a computer system comprising one or a plurality of processors which may operate together (referred to for convenience as “processor”) connected to a memory. The memory may be a non-transitory computer readable medium, such as a hard drive, a solid state disk, CD-ROM or the cloud. Software, that is executable instructions or program code, such as program code grouped into code modules, may be stored on the memory, and may, when executed by the processor, cause the computer system to perform functions such as determining that a task is to be performed to assist a user to determine the risk of a human female subject for developing breast cancer; receiving data indicating the first clinical risk assessment, the second clinical risk assessment, and the genetic risk assessment of the female subject developing breast cancer, wherein the genetic risk was derived by detecting at least two polymorphisms known to be associated with breast cancer; processing the data to combine the first clinical risk assessment, the second clinical risk assessment, and the genetic risk assessment to obtain the risk of a human female subject for developing breast cancer; outputting the risk of a human female subject for developing breast cancer.

For example, the memory may comprise program code, which when executed by the processor causes the system to determine at least two polymorphisms known to be associated with breast cancer; process the data to combine the first clinical risk assessment, the second clinical risk assessment, and the genetic risk assessment to obtain the risk of a human female subject for developing breast cancer; report the risk of a human female subject for developing breast cancer.

In another embodiment, the system may be coupled to a user interface to enable the system to receive information from a user and/or to output or display information. For example, the user interface may comprise a graphical user interface, a voice user interface or a touchscreen.

In an embodiment, the program code may cause the system to determine the “Breast Cancer Risk” (BRISK).

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

In another embodiment, performing the methods of the present disclosure to assess the risk of a human female subject for developing breast cancer, enables establishment of a diagnostic or prognostic rule based on the first clinical risk assessment, the second clinical risk assessment and the genetic risk assessment of the female subject developing breast cancer.

In an embodiment, the threshold level of risk is the level recommended by the American Cancer Society (ACS) guidelines for screening breast MRIc and mammography. In this example, the threshold level is preferably greater than about (20% lifetime risk).

In another embodiment, the threshold level of risk is the level recommended American Society of Clinical Oncology for offering an oestrogen receptor therapy to reduce a subject's risk.

In another embodiment, the diagnostic or prognostic rule is based on the application of a statistical and machine learning algorithm. Such an algorithm uses relationships between a population of polymorphisms and disease status observed in training data (with known disease status) to infer relationships which are then used to determine the risk of a human female subject for developing breast cancer in subjects with an unknown risk. An algorithm is employed which provides an risk of a human female subject developing breast cancer. The algorithm performs a multivariate or univariate analysis function.

### EXAMPLES

### Example 1—Breast Cancer Risk (BRISK) Scores Using Mammographic Density Score

First calculate a risk modifier score (risk_mod1) for each woman:

$$lnrisk 1 = ln ( PRS ) \times {β}_{PRS} + ( sqrt ( percent\_density ) - sqrt ( {mean}_{DENS} ) ) \times {β}_{DENS} + ln ( fh\_risk \_ 1 ) + ( fh\_risk \_ 2 ) + {intercept}_{PRE} + ( ( ln ( bmi / 5 ) - ln ( {mean}_{BMI} / 5 ) ) \times {β}_{PRE} ) if pre - menopausal + {intercept}_{POST} + ( ( ln ( bmi / 5 ) - ln ( {mean}_{BMI} / 5 ) ) \times {β}_{POST} ) if post - menopausal$$

then:

risk_mod1=e^{lnrisk1}

where:
fh_risk_1 and fh_risk_2 are obtained from Tables 1 and 2 respectively,
β_{PRS} is the natural logarithm of the relative risk associated with a one-unit increase in the PRS (β_{PRS}=1.03),
mean_{DENS}=the mean percent density in the population in which the test is used (mean_{DENS}=17.14),
β_{DENS} is the natural logarithm of the relative risk associated with a one-unit increase in the square root of percent density (β_{DENS}=0.12),
intercept_{PRE} is the equation intercept for pre-menopausal women (intercept_{PRE}=−0.04),
intercept_{POST} is the equation intercept for post-menopausal women (intercept_{POST}=0.05),
mean_{BMI} is the mean body mass index (kg/m²) in the population in which the test is used (mean_{BMI}=25.27),
β_{PRE} is the natural logarithm of the relative risk associated with being pre-menopausal (β_{PRE}=−0.35), and
β_{POST} is the natural logarithm of the relative risk associated with being post-menopausal (β_{POST}=0.88).

Next, calculate cumulative risks as follows:

$$cumul1\_b = 1 - {e}^{- risk \_ mod 1 \times incid \_ b} \\ cumul1\_b \_N = 1 - {e}^{- risk \_ mod 1 \times incid \_ b \_ N} \\ cumul1\_full \_life = 1 - {e}^{- risk \_ mod 1 \times incid \_ full \_ life}$$

Where incid_b, incid_b_N and incid_full_life are derived from publically available country- and ethnicity-specific population-based breast cancer incidence data (such as provided in Tables 3 to 7).

Then calculate the two BRISK scores as follows:

### 5-Year Risk

$$brisk1\_N = \frac{( cumul1\_b \_N - cumul1\_b )}{( 1 - cumul1\_b )}$$

### Remaining Lifetime Risk (to Age 90 Years)

$$brisk1\_rem \_life = \frac{( cumul1\_full \_life - cumul1\_b )}{( 1 - cumul1\_b )}$$

### Where:

N is the number of years of risk to calculate,
incid_b is the ethnicity-specific cumulative breast cancer incidence up to current age,
incid_bN is the ethnicity-specific cumulative breast cancer incidence up to current age plus N years, and
incid_full_life is the ethnicity-specific cumulative breast cancer incidence up to 90 years (i.e. full-lifetime risk).

### Example 2—Breast Cancer Risk (BRISK) Scores Using BI-RADS

First calculate a risk modifier score (risk_mod2) for each woman:

$$lnrisk 2 = ln ( PRS ) \times {β}_{PRS} + X if BI - RADS category is A or B + Y if BI - RADS category is C or D + ln ( fh\_risk \_ 1 ) + ln ( fh\_risk \_ 2 ) + {intercept}_{PRE} + ( ( ln ( bmi / 5 ) - ln ( {mean}_{BMI} / 5 ) ) \times {β}_{PRE} ) if pre - menopausal + {intercept}_{POST} + ( ( ln ( bmi / 5 ) - ln ( {mean}_{BMI} / 5 ) ) \times {β}_{POST} ) if post - menopausal$$

then:

risk_mod2=e^{lnrisk2}

where
fh_risk_1 and fh_risk_2 are obtained from Tables 1 and 2 respectively,
β_{PRS} is the natural logarithm of the relative risk associated with a one-unit increase in the PRS (β_{PRS}=1.03),
intercept_{PRE} is the equation intercept for pre-menopausal women (intercept_{PRE}=−0.04),
intercept_{POST} is the equation intercept for post-menopausal women (intercept_{POST}=0.05),
mean_{BMI} is the mean body mass index (kg/m²) in the population in which the test is used (mean_{BMI}=25.27),
β_{PRE} is the natural logarithm of the relative risk associated with being pre-menopausal (β_{PRE}=−0.35),
β_{POST} is the natural logarithm of the relative risk associated with being post-menopausal (β_{POST}=0.88).

Next, calculate cumulative risks as follows:

$$cumul2\_b = 1 - {e}^{- risk \_ mod 2 \times incid \_ b} \\ cumul2\_b \_N = 1 - {e}^{- risk \_ mod 2 \times incid \_ b \_ N} \\ cumul2\_full \_life = 1 - {e}^{- risk \_ mod 2 \times incid \_ full \_ life}$$

Where incid_b, incid_b_N and incid_full_life are derived from publically available country- and ethnicity-specific population-based breast cancer incidence data (such as provided in Tables 3 to 7).

Then calculate the two BRISK scores as follows:

### 5-Year Risk

$$brisk2\_N = \frac{( cumul2\_b \_N - cumul2\_b )}{( 1 - cumul2\_b )}$$

### Remaining Lifetime Risk (to Age 85 Years)

$$brisk2\_rem \_life = \frac{( cumul2\_full \_life - cumul2\_b )}{( 1 - cumul2\_b )}$$

### Where:

N is the number of years of risk to calculate,
incid_b is the ethnicity-specific cumulative breast cancer incidence up to current age,
incid_b_N is the ethnicity-specific cumulative breast cancer incidence up to current age plus N years, and
incid_full_life is the ethnicity-specific cumulative breast cancer incidence up to 90 years (i.e. full-lifetime risk).

### Example 3—Test Performance

The inventors tested the performance of the risk model using 200,559 eligible female participants of the UK Biobank prospective cohort study. Participants were female, genetically Caucasian and aged 40 to 69 years at baseline assessment date. It is noteworthy, that the dataset did not contain mammographic density and only incomplete family history data. Therefore, the discriminatory accuracy will be even better than that portrayed in this example.

The inventors tested the discriminatory accuracy of three versions of the risk model: incorporating a polygenic risk score comprising 77, 313, or 3,820 SNPs (Tables 11, 18 and 19). The results are provided in Table 19.

TABLE 19
Area under the receiver operating characteristic curve
(AUC) and 95% confidence interval (CI) for various
polygenic risk scores incorporated into the model.

| Risk Algorithm | AUC | (95% CI) |
|---|---|---|
| BRISK + 77 SNPs 5-year risk | 0.628 | (0.620, 0.636) |
| BRISK + 77 SNPs full lifetime risk | 0.623 | (0.615, 0.632) |
| BRISK + 313 SNPs 5-year risk | 0.648 | (0.639, 0.656) |
| BRISK + 313 SNPs full lifetime risk | 0.643 | (0.635, 0.651) |
| BRISK + 3820 SNPs 5-year risk | 0.650 | (0.641, 0.658) |
| BRISK + 3820 SNPs full lifetime risk | 0.646 | (0.639, 0.654) |

The inventors assessed calibration using logistic regression of the logit of the risk score to estimate the intercept and the slope (B coefficient). An intercept close to 0 indicates good calibration, while an intercept of less than 0 indicates overall overestimation and an intercept of greater than 0 indicated overall underestimation of risk.

In terms of the dispersion of the risk score, a slope of close to 1 indicates good estimation across the spectrum of risk. A slope of less than 1 means that the predicted probabilities do not vary enough (i.e. underestimation of true high risk and overestimation of true low risk). Conversely, a slope of greater than one means that the predicted probabilities vary too much (i.e. underestimation of true low risk and overestimation of true high risk).

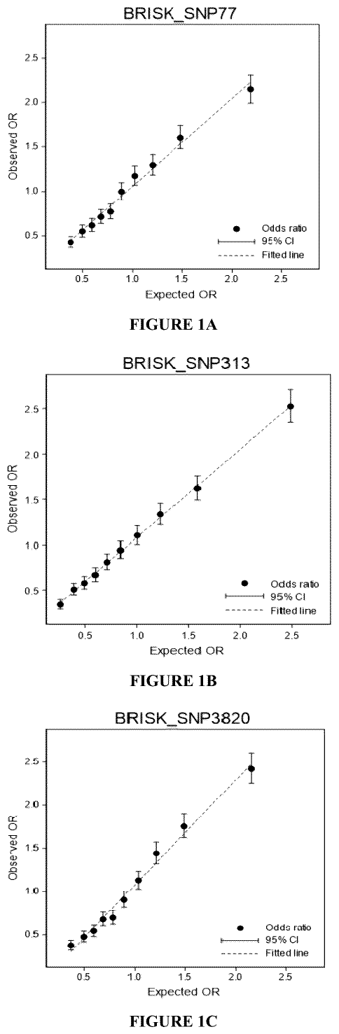

Table 20 and FIG. 1 show the calibration analysis for the three risk models.

TABLE 20
Slope and intercept values of the calibration
plots for each of the risk models.

| Risk Algorithm | Slope | Intercept |
|---|---|---|
| Brisk + 77 SNPs | 1.000 | 0.068 |
| Brisk + 313 SNPs | 1.026 | 0.081 |
| Brisk + 3,820 SNPs | 0.813 | 0.104 |

The present application claims priority from AU 2021903955 filed 7 Dec. 2021, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Adessi et al. (2000) Nucl. Acids Res. 28, E87.
Antoniou et al. (2008) Br J Cancer 98:1457-1466.
Bennett et al. (2005) Pharmacogenomics 6:373-382.
Berry et al. (1997) J Natl Cancer Inst 89:227-238.
Birren et al. (1997) Genome Analysis: Analyzing DNA, A Laboratory Manual Vol 1.
Brenner et al. (2000) Nat. Biotechnol. 18:630-634.
Boyd et al. (2002) N Engl J Med 347:886-894.
Byng et al. (1994) Phys Med Biol 39:1629-1638.
Byng et al. (1998) Radiographics 18:1587-1598.
Cappello et al. (2019) J Am Coll Radiol 16:139-146.
Chee et al. (1996) Science 274:610-614.
Collaborative Group on Hormonal Factors in Breast Cancer (2001) Lancet 358:1389-1399.
Devlin and Risch (1995) Genomics. 29:311-322.
Fodor (1997a) FASEB Journal 11: A879.
Fodor (1997b) Science 277:393-395.
Hopper et al. (2018) Breast Cancer Res 20:132.
Kuchenbaecker et al. (2017) Jama 317:2402-2416.
Lockhart (1998) Nature Medicine 4:1235-1236.
MacLean et al. (2009) Nature Rev. Microbiol, 7:287-296.
Margulies et al. (2005) Nature 437:376-380.
Mavaddat et al. (2015) J Natl Cancer Inst 107: doi: 10.1093/jnci/djv036.
Mavaddat et al. (2019) Am J Hum Genet 104:21-34.
Michailidou et al. (2017) Nature 551:92-94.
Mitra et al. (2003) Analytical Biochemistry 320:55-65.
Morozova & Marra (2008) Genomics 92:255.
Nickson et al. (2018) Breast Cancer Res 20:155.
Privé et al. (2019) The American Journal of Human Genetics 105:1213-1221.
Sak et al. (2015) Breast Cancer Manag 4:209-221.
Sapolsky et al. (1999) Genet Anal: Biomolec Engin 14:187-192.
Shendure et al. (2005) Science 309:1728-1732.
Sickles and Bassett (2013) ACR BI-RADS® Atlas, Breast Imaging and Reporting Data
System. Reston, VA: American College of Radiology.
Slatkin and Excoffier (1996) Heredity 76:377-383.
Sorlie et al. (2001) Proc. Natl. Acad. Sci. 98:10869-10874.
Tijssen (1993) Laboratory Techniques in Biochemistry and Molecular Biology-Hybridization with Nucleic Acid Probes Elsevier, New York.
Tyrer et al. (2004) Stat Med 23:1111-1130.
Voelkerding et al. (2009) Clinical Chem. 55:641-658.
Wendt and Margolin (2019) Acta Oncologica 58:135-146.
Ziogas et al. (2000) Cancer Epidemiol Biomarkers Prev 9:103-111.

1. A computer-implemented method for assessing the risk of a human female subject developing breast cancer, the method comprising:
determining a genetic risk score based on genetic data derived from a biological sample taken from the female subject;
determining a mammographic density risk score based on data derived from a clinical assessment of the mammographic density of the female subject;
determining a first-degree family history risk score and a second-degree family history risk score based on family history data of the female subject;
determining a body mass index risk score based on the body mass index and menopause status of the subject;
calculating a risk modifier score, the risk modifier score being a function of the polygenetic risk score, mammographic density risk score, first-degree family history risk score, second-degree family history risk score and body mass index risk score;
calculating at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculating the female subject's absolute risk of breast cancer in the next N years, and/or in the subjects full lifetime, using the cumulative risk scores.

2. The method of claim 1, wherein the genetic risk score is determined by ln(PRS)×β_{PRS}, where the PRS is the polygenic risk score that represents the female subject's genetic predisposition to breast cancer, and β_{PRS} is the natural logarithm of the relative risk associated with a one-unit increase in ln(PRS).

3. The method of claim 1 or claim 2, wherein the genetic risk score is determined using at least 50, at least 100, at least 150, at least 200, at least 250, at least 300, at least 313, at least 350, at least 400, at least 450, at least 500, at least 550, at least 600, at least 650, at least 700, at least 750, at least 800, at least 825, at least 850, at least 3,820 or all of the polymorphism provided in one or more of Tables 8 to 18, or a polymorphism in linkage disequilibrium with one or more thereof.

4. The method according to any one of claims 1 to 3, wherein the genetic risk score is determined using the 313 polymorphism of Table 17.

5. The method according to any one of claims 1 to 3, wherein the genetic risk score is determined using the 3,820 polymorphisms of Table 18.

6. The method according to any one of claims 1 to 5, wherein the first-degree family history risk score (fh_risk_1) is based on the subjects age and the number and youngest age affected of the subjects first-degree relatives with invasive breast cancer.

7. The method of claim 6, wherein the first-degree family history risk score is ln(fh_risk_1).

8. The method according to any one of claims 1 to 7, wherein the second-degree family history risk score (fh_risk_2) is based on the subjects age and the subject's number of second-degree relatives with invasive breast cancer.

9. The method of claim 8, wherein the second-degree family history risk score is ln(fh_risk_2).

10. The method according to any one of claims 1 to 9, wherein the body mass index risk score is determined using intercept_{PRE}+((ln(bmi/5)−ln(mean_{BMI}/5))×β_{PRE}) if pre-menopausal, or intercept_{POST}+((ln(bmi/5)−ln(mean_{BMI}/5))×β_{POST}) if post-menopausal, where mean_{BMI} is the mean body mass index in the population in which the test is used, intercept_{PRE} is the equation intercept for pre-menopausal women and intercept_{POST} is the equation intercept for post-menopausal women.

11. The method according to any one of claims 1 to 9, wherein the mammographic density risk score is determined using (sqrt(percent_density)−sqrt(mean_{DENS}))×β_{DENS}, where mean_{DENS} is the mean percent density in the population in which the test is used, and β_{DENS} is the natural logarithm of the relative risk associated with a one-unit increase in the square root of percent density.

12. The method according to any one of claims 1 to 11, wherein the risk modifier score (risk_mod1) is determined using:
$$lnrisk 1 = ln ( PRS ) \times {β}_{PRS} + ( sqrt ( percent\_density ) - sqrt ( {mean}_{DENS} ) ) \times {β}_{DENS} + ln ( fh\_risk \_ 1 ) + ( fh\_risk \_ 2 ) + {intercept}_{PRE} + ( ( ln ( bmi / 5 ) - ln ( {mean}_{BMI} / 5 ) ) \times {β}_{PRE} ) if pre - menopausal + {intercept}_{POST} + ( ( ln ( bmi / 5 ) - ln ( {mean}_{BMI} / 5 ) ) \times {β}_{POST} ) if post - menopausal$$
then:
risk_mod1=e^{lnrisk1}.

13. The method of claim 12, wherein the at least two cumulative risk scores are selected from:
$$cumul1\_b = 1 - {e}^{- risk \_ mod 1 \times incid \_ b} \\ cumul1\_b \_N = 1 - {e}^{- risk \_ mod 1 \times incid \_ b \_ N} \\ cumul1\_full \_life = 1 - {e}^{- risk \_ mod 1 \times incid \_ full \_ life}$$
where:
N is the number of years of risk to calculate
incid_b=is the ethnicity-specific cumulative breast cancer incidence up to current age,
incid_bN5=is the ethnicity-specific cumulative breast cancer incidence up to current age plus 5N years, and
incid_full_life is the ethnicity-specific cumulative breast cancer incidence up to 90 years.

14. The method of claim 12, wherein the breast cancer risk score is determined using either of:
$$brisk1\_N = \frac{( cumul1\_b \_N - cumul1\_b )}{( 1 - cumul1\_b )}$$
$$or$$
$$brisk1\_rem \_life = \frac{( cumul1\_full \_life - cumul1\_b )}{( 1 - cumul1\_b )} .$$

15. The method according to any one of claims 1 to 9, wherein the mammographic density risk score is a BI-RADS category.

16. The method according to any one of claim 1 to 9 or 15, wherein the risk modifier score (risk_mod2) is determined using:
$$lnrisk 1 = ln ( PRS ) \times {β}_{PRS} + ( sqrt ( percent\_density ) - sqrt ( {mean}_{DENS} ) ) \times {β}_{DENS} + ln ( fh\_risk \_ 1 ) + ( fh\_risk \_ 2 ) + {intercept}_{PRE} + ( ( ln ( bmi / 5 ) - ln ( {mean}_{BMI} / 5 ) ) \times {β}_{PRE} ) if pre - menopausal + {intercept}_{POST} + ( ( ln ( bmi / 5 ) - ln ( {mean}_{BMI} / 5 ) ) \times {β}_{POST} ) if post - menopausal$$
wherein, X is between −0.05 and −0.25, and Y is between 0.14 and 0.34,
then:
risk_mod2=e^{lnrisk2}.

17. The method of claim 16, wherein the at least two cumulative risk scores are selected from:
$$cumul2\_b = 1 - {e}^{- risk \_ mod 2 \times incid \_ b} \\ cumul2\_b \_N = 1 - {e}^{- risk \_ mod 2 \times incid \_ b \_ N} \\ cumul2\_full \_life = 1 - {e}^{- risk \_ mod 2 \times incid \_ full \_ life}$$
where:
N is the number of years of risk to calculate,
incid_b=is the ethnicity-specific cumulative breast cancer incidence up to current age,
incid_b_N5=is the ethnicity-specific cumulative breast cancer incidence up to current age plus 5N years, and
incid_full_life is the ethnicity-specific cumulative breast cancer incidence up to 90 years (i.e. full-lifetime risk).

18. The method of claim 12, wherein the breast cancer risk score is determined using either of:
$$brisk2\_N = \frac{( cumul2\_b \_N - cumul2\_b )}{( 1 - cumul2\_b )}$$
$$or$$
$$brisk2\_rem \_life = \frac{( cumul2\_full \_life - cumul2\_b )}{( 1 - cumul2\_b )} .$$

19. The method according to any one of claims 1 to 18 which further comprises comparing the score to a pre-determined threshold.

20. The method according to any one of claims 1 to 19 which further comprises outputting the breast cancer risk score.

21. A computer-implemented method for assessing the risk of a human female subject developing breast cancer, the method comprising:
determining a mammographic density risk score based on data derived from a clinical assessment of the mammographic density of the female subject;
determining a first-degree family history risk score and a second-degree family history risk score based on family history data of the female subject;
determining a body mass index risk score based on the body mass index and menopause status of the subject;
calculating a risk modifier score, the risk modifier score being a function of the mammographic density risk score, first-degree family history risk score, second-degree family history risk score and body mass index risk score;
calculating at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculating the female subject's absolute risk of breast cancer in the next N years, and/or in the subjects full lifetime, using the cumulative risk scores.

22. A computer readable storage medium storing executable code, wherein when a processor executes the code, the processor is cause to perform the method of any one of claims 1 to 21.

23. A device for assessing the risk of a human female subject developing breast cancer, the device comprising:
a processor; and
a memory device storing executable code, the memory being accessible to the processor;
wherein when caused to execute the executable code stored in the memory device, the processor is caused to:
determine a genetic risk score based on genetic data derived from a biological sample taken from the female subject;
determine a mammographic density risk score based on data derived from a clinical assessment of the mammographic density of the female subject;
determine a first-degree family history risk score and a second-degree family history risk score based on family history data of the female subject;
determine a body mass index risk score based on the body mass index and menopause status of the subject;
calculate a risk modifier score, the risk modifier score being a function of the polygenetic risk score, mammographic density risk score, first-degree family history risk score, second-degree family history risk score and body mass index risk score
calculate at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculate the female subject's absolute risk of breast cancer in the next N years, and/or in the subjects full lifetime, using the cumulative risk scores.

24. A device for assessing the risk of a human female subject developing breast cancer, the device comprising:
a processor; and
a memory device storing executable code, the memory being accessible to the processor;
wherein when caused to execute the executable code stored in the memory device, the processor is caused to:
determine a mammographic density risk score based on data derived from a clinical assessment of the mammographic density of the female subject;
determine a first-degree family history risk score and a second-degree family history risk score based on family history data of the female subject;
determine a body mass index risk score based on the body mass index and menopause status of the subject;
calculate a risk modifier score, the risk modifier score being a function of the mammographic density risk score, first-degree family history risk score, second-degree family history risk score and body mass index risk score
calculate at least two cumulative risk scores of the female subject developing breast cancer to her current age, to the next N years and/or full lifetime, the cumulative risk being a function of the risk modifier score and a cumulative breast cancer incidence value based on the subjects age and ethnicity, and
calculate the female subject's absolute risk of breast cancer in the next N years, and/or in the subjects full lifetime, using the cumulative risk scores.

25. The device of claim 23 or claim 24, further comprising a display component, wherein the processor is further caused to display the breast cancer risk score of the female subject for developing breast cancer on the display component.

26. The device according to any one of claims 23 to 25, further comprising a communications module, wherein the processor is further caused to communicate the breast cancer risk score of the female subject for developing breast cancer to an external device via the communications module.

27. A method for assessing the risk of a human female subject for developing breast cancer comprising:
i) determining the age, ethnicity, family history of breast cancer incorporating number of first-degree relatives who have had breast cancer, the age of the youngest first-degree relative at diagnosis with breast cancer, the number of second-degree relatives who have had breast cancer, country of residence, mammographic breast density, body mass index (BMI) and menopausal status,
ii) determining the genotype of the human female subject at 50 or more polymorphic loci associated with a risk of a human female subject for developing breast cancer,
iii) determining a breast cancer risk score based on i) and ii).

28. A method for assessing the risk of a human female subject for developing breast cancer comprising:
i) determining the age, ethnicity, family history of breast cancer incorporating number of first-degree relatives who have had breast cancer, the age of the youngest first-degree relative at diagnosis with breast cancer, the number of second-degree relatives who have had breast cancer, country of residence, mammographic breast density, body mass index (BMI) and menopausal status,
ii) determining a breast cancer risk score based on i).

29. The method according to any one of claim 1 to 21, 27 or 28, wherein the results of the assessment indicate that the female should be subjected to more frequent screening and/or prophylactic anti-breast cancer therapy.

30. The method according to any one of claim 1 to 21, or 27 or 29, wherein if it is determined that the subject has a risk of developing breast cancer, the subject is more likely to be responsive to oestrogen inhibition therapy than non-responsive.

31. The method according to any one of claims 1 to 21 or 27 to 30, wherein the breast cancer is oestrogen receptor positive or oestrogen receptor negative.

32. A method for determining the need for routine diagnostic testing of a human female subject for breast cancer comprising assessing the risk of the subject for developing breast cancer using the method according to any one of claims 1 to 21 or 27 to 31.

33. The method of claim 32, wherein an risk score greater than about 20% lifetime risk indicates that the subject should be enrolled in a screening breast MRI and mammography program.

34. A method of screening for breast cancer in a human female subject, the method comprising assessing the risk of the subject for developing breast cancer using the method according to any one of claims 1 to 21 or 27 to 31, and routinely screening for breast cancer in the subject if they are assessed as having a risk for developing breast cancer.

35. A method for determining the need of a human female subject for prophylactic anti-breast cancer therapy comprising assessing the risk of the subject for developing breast cancer using the method according to any one of claims 1 to 21 or 27 to 31.

36. The method of claim 35, wherein an risk score greater than about 1.66% 5-year risk indicates that oestrogen receptor antagonist therapy should be offered to the subject.

37. A method for preventing breast cancer in a human female subject, the method comprising assessing the risk of the subject for developing breast cancer using the method according to any one of claims 1 to 21 or 27 to 31, and administering an anti-breast cancer therapy to the subject if they are assessed as having a risk for developing breast cancer.

38. The method of claim 37, wherein the therapy inhibits oestrogen.

39. An anti-breast cancer therapy for use in preventing breast cancer in a human female subject at risk thereof, wherein the subject is assessed as having a risk for developing breast cancer according to the method of any one of claims 1 to 21 or 27 to 31.

40. A method for stratifying a group of human female subjects for a clinical trial of a candidate therapy, the method comprising assessing the individual risk of the subjects for developing breast cancer using the method according to any one of claims 1 to 21 or 27 to 31, and using the results of the assessment to select subjects more likely to be responsive to the therapy.
